(19) United States
(12) Patent Application Publication
(10) Pub. No.: US 2025/0228854 A1
(43) Pub. Date: Jul. 17, 2025

(54) HETEROCYCLIC SUBSTITUTED PYRIMIDOPYRAN COMPOUNDS AND THEIR APPLICATIONS

(71) Applicant: D3 Bio (Wuxi) Co., Ltd., Wuxi (CN)

(72) Inventors: Zhijian CHEN, Shanghai (CN); Jing ZHANG, Shanghai (CN); Robert A. MOOK, JR., Chapel Hill, NC (US); Tienan WANG, Shanghai (CN); Haibo XIE, Shanghai (CN); Jiuxiang ZHU, Shanghai (CN)

(21) Appl. No.: 19/096,452

(22) Filed: Mar. 31, 2025

Related U.S. Application Data
(63) Continuation of application No. PCT/CN2025/082991, filed on Mar. 17, 2025.

(30) Foreign Application Priority Data
Mar. 23, 2023 (CN) 202310290481.3
Nov. 30, 2023 (CN) 202311634664.9
Mar. 18, 2024 (CN) 202410309827.4
Mar. 22, 2024 (WO) PCT/CN2024/083328

(51) Int. Cl. A61K 31/506; C07D 519/00

(52) U.S. Cl. CPC A61K 31/506; C07D 519/00

(57) ABSTRACT

The present disclosure relates to a class of pyrimidopyran compounds substituted with piperazine bridge and their applications thereof, such as compounds of Formulae (I″), (I′), or (I), stereoisomers thereof, or pharmaceutically acceptable salts thereof.

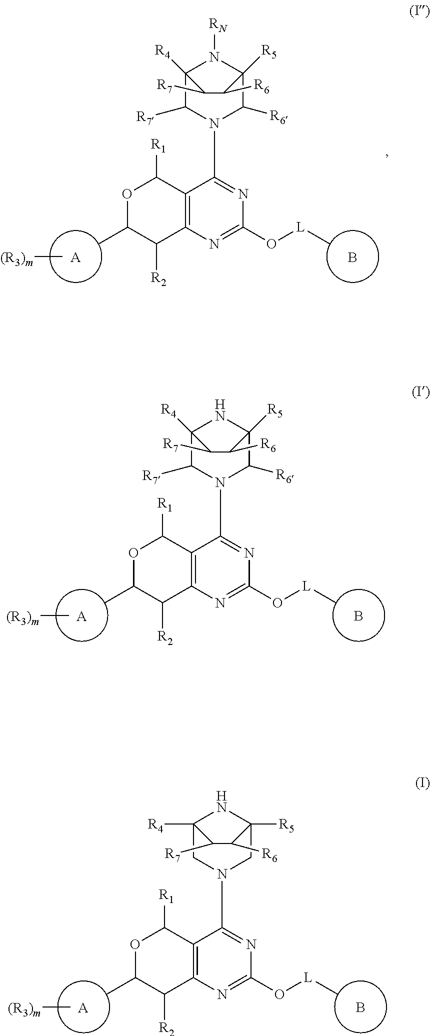

### RELATED APPLICATIONS

This application is a continuation of International Patent Application No. PCT/CN2025/082991, filed on Mar. 17, 2025, which claims the benefit of and priority to International Patent Application No. PCT/CN2024/083328, filed on Mar. 22, 2024, which claims the benefit of and priority to Chinese Application Nos. 202310290481.3, filed Mar. 23, 2023, 202311634664.9, filed Nov. 30, 2023, and 202410309827.4, filed Mar. 18, 2024, the contents of which are incorporated herein by reference in their entirety.

### TECHNICAL FIELD

The present disclosure is directed to a class of piperazine bridging ring substituted pyrimidopyran compounds and their applications, in particular to compounds of Formula (I″), Formula (I′), or Formula (I), stereoisomers thereof, and pharmaceutically acceptable salts thereof.

### BACKGROUND

KRAS is the most common oncogenic mutation gene, and KRAS mutations occur in about 1 in 7 cancers KRAS mutation/KRAS amplification is most common in colorectal cancer (US: ˜45% China: ˜49%), pancreatic cancer (US: ˜90% China: ˜87% non-small cell lung cancer (US: ˜35% China: ˜13%). Among them, KRAS^{G12D}, KRAS^{G12V} and KRAS^{G12C} account for the largest proportion. KRAS is a murine sarcoma virus oncogene and an important member of the RAS protein. KRAS acts as a molecular switch that regulates the path of cell growth when its function is normal; after KRAS gene mutation, it can independently transmit growth and proliferation signals to downstream pathways independent of upstream growth factor receptor signals, resulting in uncontrolled cell growth and tumor progression. At the same time, whether the KRAS gene is mutated is also an important indicator of tumor prognosis.

At present, small molecules that directly target KRAS mutations are mainly concentrated in the field of KRAS^{G12C}. Among them, Amgen's AMG510 and Mirati Therapeutics' MRTX849 have both been approved for marketing, and have shown good therapeutic effects in patients with KRAS^{G12C}-mutant tumors. MRTX1133, as a small molecule drug targeting KRAS^{G12D} mutation, has entered clinical phase I, and it has shown excellent anti-tumor properties in preclinical studies. However, there are still some problems with this class of compounds. Tumor patients with KRAS^{G12D} mutations have not yet benefited from precision medicine, and the continued development of small molecule inhibitors targeting KRAS^{G12D} is still of great significance.

A series of small molecule inhibitors targeting KRAS^{G12D} and methods of making the same are disclosed.

### SUMMARY

In certain aspects, the present disclosure provides a compound of formula (I″):

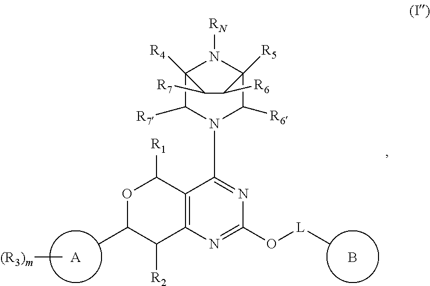

or a stereoisomer thereof, or pharmaceutically acceptable salt thereof, wherein each of the variable in Formula (I″) is described, embodied, and exemplified herein.

In certain aspects, the present disclosure provides pharmaceutical compositions comprising a compound disclosed herein or a stereoisomer thereof, or a pharmaceutically acceptable salt thereof, and a pharmaceutically acceptable excipient.

In certain aspects, the present disclosure provides methods of treating or preventing a disease or disorder (e.g., a disease or disorder associated with dysregulation and/or mutation of KRAS) in a subject in need thereof, comprising administering to the subject a compound disclosed herein or a stereoisomer thereof, or a pharmaceutically acceptable salt thereof (e.g., in a therapeutically effective amount).

In certain aspects, the present disclosure provides methods of treating a disease or disorder (e.g., a disease or disorder associated with dysregulation and/or mutation of KRAS) in a subject in need thereof, comprising administering to the subject a compound disclosed herein or a stereoisomer thereof, or a pharmaceutically acceptable salt thereof (e.g., in a therapeutically effective amount).

In certain aspects, the present disclosure provides uses of a compound disclosed herein or a stereoisomer thereof, or a pharmaceutically acceptable salt thereof in the manufacture of a medicament for treating or preventing a disease or disorder (e.g., a disease or disorder associated with dysregulation and/or mutation of KRAS) in a subject in need thereof.

In certain aspects, the present disclosure provides uses of a compound disclosed herein or a stereoisomer thereof, or a pharmaceutically acceptable salt thereof in the manufacture of a medicament for treating a disease or disorder (e.g., a disease or disorder associated with dysregulation and/or mutation of KRAS) in a subject in need thereof.

In certain aspects, the present disclosure provides compounds disclosed herein or a stereoisomer thereof, or a pharmaceutically acceptable salt thereof for use in treating or preventing a disease or disorder (e.g., a disease or disorder associated with dysregulation and/or mutation of KRAS) in a subject in need thereof.

In certain aspects, the present disclosure provides compounds disclosed herein or a stereoisomer thereof, or a pharmaceutically acceptable salt thereof for use in treating a disease or disorder (e.g., a disease or disorder associated with dysregulation and/or mutation of KRAS) in a subject in need thereof.

### DETAILED DESCRIPTION

The present disclosure relates to compounds and compositions that are useful as pan-KRAS inhibitors. The present disclosure also relates to methods of treating a disease or disorder in a subject in need thereof by administering (e.g., in a therapeutically effective amount) a compound disclosed herein. The present disclosure further relates to methods of treating a disease or disorder in a subject in need thereof, comprising administering (e.g., in a therapeutically effective amount) a pharmaceutical composition comprising a compound disclosed herein.

### Compounds of the Application

In certain aspects, the present disclosure provides a compound of Formula (I″):

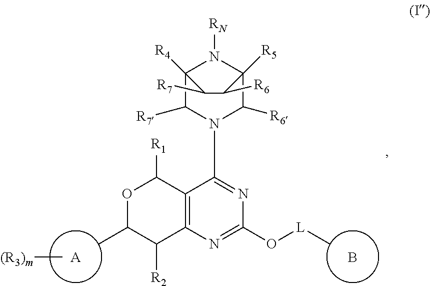

a stereoisomer thereof, or a pharmaceutically acceptable salt thereof, wherein:
R_{N} is selected from H and C₁₋₃ alkyl, wherein the C₁₋₃ alkyl is optionally substituted with 1, 2, or 3 F or Cl;
Ring A is selected from C₆ aryl and 5-6 membered heteroaryl;
Ring B is selected from

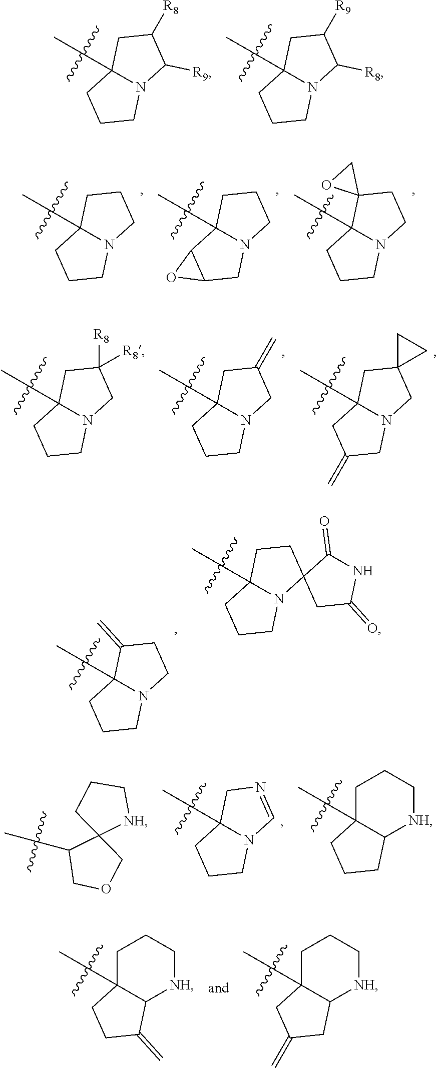

wherein Ring B is optionally substituted with 1, 2, 3, or 4 R₁₀;
L is —C(R_{L1}R_{L2})—, wherein R_{L1} and R_{L2} are each independently selected from H, D, and C₁₋₃ alkyl;
R₁ and R₂ are each independently selected from oxo, H, F, Cl, Br, I, and CN;
each R₃ is independently selected from F, Cl, Br, I, OH, NH₂, CN, C₁₋₃ alkyl, C₁₋₃ alkoxy, C₁₋₃ alkylamino, di-C₁₋₃ alkylamino, C₂₋₄ alkenyl, C₂₋₄ alkynyl, and C₃₋₅ cycloalkyl, wherein the C₁₋₃ alkyl, C₁₋₃ alkoxy, C₁₋₃ alkylamino, di-C₁₋₃ alkylamino, C₂₋₄ alkenyl, C₂₋₄ alkynyl, and C₃₋₅ cycloalkyl are each independently optionally substituted with 1, 2, 3, 4, or 5 Rₐ;
each Rₐ is independently selected from D, F, Cl, Br, and I;
R₄, R₅, R₆, R₇, R_{6′}, and R_{7′} are each independently selected from oxo, H, F, Cl, Br, I, OH, NH₂, CN, C₁₋₃ alkyl, C₂₋₄ alkenyl, C₁₋₃ alkoxy, —C(═O)—R_{d}, —C(═O)—NR_{b1}R_{b2}, and =NO(C₁₋₃ alkyl), wherein the C₁₋₃ alkyl, C₂₋₄ alkenyl, and C₁₋₃ alkoxy are each independently optionally substituted with 1, 2, 3, 4, or 5 R_{b};
or, R₆ and R₇ together with the carbon atoms to which they are attached form a 3-5 membered heterocyclyl;
each R_{b} is independently selected from D, F, Cl, Br, I, OH, NH₂, CN, C₁₋₃ alkoxy, and —C(═O)—NR_{b1}R_{b2};
R₈ is selected from H, F, Cl, Br, I, OH, NH₂, CN, C₁₋₃ alkyl, and C₁₋₃ alkoxy, wherein the C₁₋₃ alkyl and C₁₋₃ alkoxy are each independently optionally substituted with 1, 2, 3, 4, or 5 Rₐ;
or, R₈ and R_{8′} together with the carbon atom to which they are attached form a C₃₋₅ cycloalkyl or 3-5 membered heterocyclyl, wherein the C₃₋₅ cycloalkyl and 3-5 membered heterocyclyl are each independently optionally substituted with 1, 2, or 3 R₁₀;
R₉ is selected from —C(═O)—NR_{b3}R_{b4} and —CH₂R_{c};
each R₁₀ is independently selected from oxo, D, F, Cl, Br, I, OH, NH₂, CN, C₁₋₃ alkyl, C₁₋₃ alkoxy, C₁₋₃ alkylamino, di-C₁₋₃ alkylamino, —C(═O)—R_{d}, —S—R_{d}, —S(═O)—R_{d}, —S(═O)₂—R_{d}, —NH—C(═O)—R_{d}, C₆₋₁₀ aryl, and 5-10 membered heteroaryl, wherein the C₁₋₃ alkyl is optionally substituted with 1, 2, or 3 OH or F, and the C₆₋₁₀ aryl and 5-10 membered heteroaryl are each independently optionally substituted with 1, 2, 3, 4, or 5 Rₛ₁;
R_{b1} and R_{b2} are each independently selected from H, C₁₋₆ alkyl, C₁₋₆ alkoxy, C₃₋₁₀ cycloalkyl, 3-10 membered heterocyclyl, C₆₋₁₀ aryl, and 5-10 membered heteroaryl, wherein the C₁₋₆ alkyl, C₁₋₆ alkoxy, C₃₋₁₀ cycloalkyl, 3-10 membered heterocyclyl, C₆₋₁₀ aryl, and 5-10 membered heteroaryl are each independently optionally substituted with 1, 2, 3, or 4 Rₑ₁;
or, R_{b1} and R_{b2} together with the nitrogen atom to which they are attached form a 3-6 membered heterocyclyl, wherein the 3-6 membered heterocyclyl is optionally substituted with 1, 2, 3, or 4 Rₑ₁;
R_{b3} and R_{b4} are each independently selected from H, C₁₋₆ alkyl, C₁₋₆ alkoxy, C₃₋₁₀ cycloalkyl, 3-10 membered heterocyclyl, C₆₋₁₀ aryl, and 5-10 membered heteroaryl, wherein the C₁₋₆ alkyl, C₁₋₆ alkoxy, C₃₋₁₀ cycloalkyl, 3-10 membered heterocyclyl, C₆₋₁₀ aryl, and 5-10 membered heteroaryl are each independently optionally substituted with 1, 2, 3, or 4 Rₑ₂;
or, R_{b3} and R_{b4} together with the nitrogen atom to which they are attached form a 3-6 membered heterocyclyl, wherein the 3-6 membered heterocyclyl is optionally substituted with 1, 2, 3, or 4 Rₑ₂; R_{c} is selected from F, Cl, Br, I, OH, NH₂, —(C═O)NR_{C1}R_{C2}, —O(C═O)NR_{C1}R_{C2}, —NR_{C0}(C═O)R_{C1}, and —NR_{C0}(C═O)NR_{C1}R_{C2};
R_{C1}, R_{C1}, and R_{C2} are each independently selected from H, C₁₋₆ alkyl, C₃₋₆ cycloalkyl, and 3-6 membered heterocyclyl;
R_{d} is C₁₋₃ alkyl;
Rₑ₁ is selected from F, Cl, Br, I, OH, NH₂, NO₂, C₁₋₃ alkyl, C₁₋₃ alkylamino, di-C₁₋₃ alkylamino, CN, C₁₋₃ alkoxy, —S(═O)₂—(C₁₋₃ alkyl), —(C═O)(C₁₋₃ alkyl), —(C=O)O(C₁₋₃ alkyl), —(C═O)NH(C₁₋₃ alkyl), —(C═O)N(C₁₋₃ alkyl)₂, C₃₋₁₀ cycloalkyl, 3-10 membered heterocyclyl, C₆₋₁₀ aryl, and 5-10 membered heteroaryl;
Rₑ₂ is selected from F, Cl, Br, I, OH, NH₂, NO₂, C₁₋₃ alkyl, C₁₋₃ alkylamino, di-C₁₋₃ alkylamino, CN, C₁₋₃ alkoxy, —S(═O)₂—(C₁₋₃ alkyl), —(C═O)(C₁₋₃ alkyl), —(C=O)O(C₁₋₃ alkyl), —(C═O)NH(C₁₋₃ alkyl), —(C═O)N(C₁₋₃ alkyl)₂, C₃₋₁₀ cycloalkyl, 3-10 membered heterocyclyl, C₆₋₁₀ aryl, and 5-10 membered heteroaryl, wherein the C₃₋₁₀ cycloalkyl, 3-10 membered heterocyclyl, C₆₋₁₀ aryl, and 5-10 membered heteroaryl are each independently optionally substituted with 1, 2, 3, 4, or 5 Rₛ₁, and wherein the C₁₋₃ alkyl and C₁₋₃ alkoxy are each independently optionally substituted with 1, 2, 3, 4, or 5 Rₛ₂;
or, two Rₑ₂ together with the carbon atoms to which they are attached form a C₆ aryl or 5- or 6-membered heteroaryl;
Rₛ₁ is selected from oxo, F, Cl, Br, I, OH, NH₂, NO₂, C₁₋₆ alkyl, C₁₋₆ alkylamino, di-C₁₋₆ alkylamino, CN, C₁₋₆ alkoxy, —S(═O)₂—(C₁₋₃ alkyl), —(C═O)(C₁₋₃ alkyl), —(C═O)O(C₁₋₃ alkyl), —(C═O)NH(C₁₋₃ alkyl), and —(C═O)N(C₁₋₃ alkyl)₂;
Rₛ₂ is selected from F, Cl, Br, I, OH, NH₂, C₁₋₆ alkylamino, di-C₁₋₆ alkylamino, CN, C₁₋₆ alkoxy, —S(═O)₂—(C₁₋₃ alkyl), —(C═O)(C₁₋₃ alkyl), —(C═O)O(C₁₋₃ alkyl), —(C═O)NH(C₁₋₃ alkyl), and —(C═O)N(C₁₋₃ alkyl)₂; and
m is selected from 0, 1, 2, 3, 4, and 5;
provided that,
1) when Ring B is

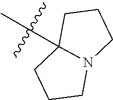 [SMILES: *C12CCCN1CCC2]

substituted with one R₁₀ and R₁₀ is F, at least one of R₁, R₂, R₄, R₅, R₆, R₇, R_{6′}, and R_{7′} is not H;
2) when Ring B is

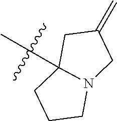 [SMILES: *C12CCCN1CC(=C)C2]

optionally substituted with 1, 2, 3, or 4 R₁₀, at least one of R₁, R₂, R₄, R₅, R₆, R₇, R_{6′}, and R_{7′} is not H; and
3) the compound is not

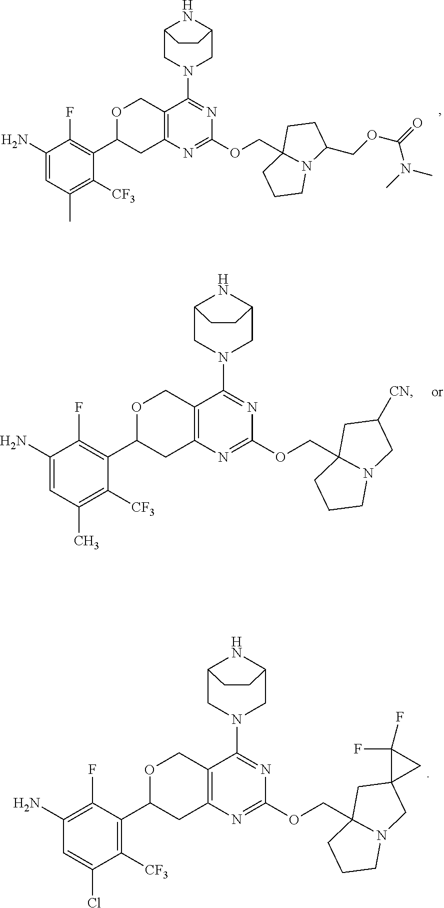 [SMILES: Cc1cc(N)c(F)c(C2Cc3nc(OCC45CCCN4C(COC(=O)N(C)C)CC5)nc(N4CC5CCC(C4)N5)c3CO2)c1C(F)(F)F ; Cc1cc(N)c(F)c(C2Cc3nc(OCC45CCCN4CC(C#N)C5)nc(N4CC5CCC(C4)N5)c3CO2)c1C(F)(F)F ; Nc1cc(Cl)c(C(F)(F)F)c(C2Cc3nc(OCC45CCCN4CC4(C5)CC4(F)F)nc(N4CC5CCC(C4)N5)c3CO2)c1F]

In some embodiments, R_{N} is H.

In some embodiments, R_{N} is C₁₋₃ alkyl optionally substituted with 1, 2, or 3 F or Cl.

In some embodiments, the present disclosure provides a compound of Formula (I′):

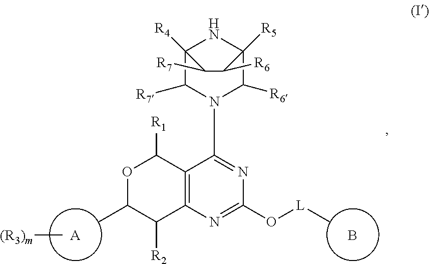

a stereoisomer thereof, or a pharmaceutically acceptable salt thereof, wherein:
Ring A is selected from the group consisting of C₆ aryl and 5-6 membered heteroaryl;
Ring B is selected from the group consisting of

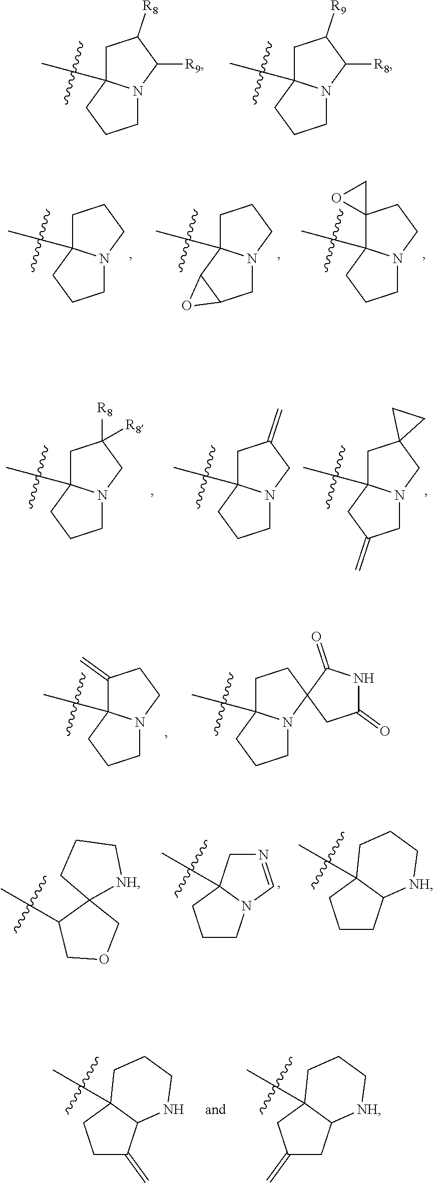

wherein Ring B is optionally substituted with 1, 2, 3, or 4 R₁₀;
L is —CH₂— optionally substituted with 1 or 2 D;
R₁ and R₂ are each independently selected from oxo, H, F, Cl, Br, I, and CN;
each R₃ is independently selected from F, Cl, Br, I, OH, NH₂, CN, C₁₋₃ alkyl, C₁₋₃ alkoxy, C₁₋₃ alkylamino, diC₁₋₃ alkylamino, C₂₋₄ alkenyl, C₂₋₄ alkynyl, and C₃₋₅ cycloalkyl, wherein the C₁₋₃ alkyl, C₁₋₃ alkoxy, C₁₋₃ alkylamino, di-C₁₋₃alkylamino, C₂₋₄ alkenyl, C₂₋₄ alkynyl and C₃₋₅ cycloalkyl are each independently substituted with 1, 2, 3, 4, or 5 Rₐ,
each Rₐ is independently selected from D, F, Cl, Br, and I;
R₄, R₅, R₆, R₇, R_{6′}, and R_{7′} are each independently selected from oxo, H, F, Cl, Br, I, OH, NH₂, CN, C₁₋₃ alkyl, C₂₋₄ alkenyl, C₁₋₃ alkoxy, —C(═O)—R_{d}, —C(═O)—NR_{b1}R_{b2}, and ═NO(C₁₋₃ alkyl), wherein the C₁₋₃ alkyl, C₂₋₄ alkenyl, and C₁₋₃ alkoxy are each independently optionally substituted with 1, 2, 3, 4, or 5 R_{b};
or R₆ and R₇ together with the carbon atoms to which they are attached form a 3-5 membered heterocyclyl;
each R_{b} is independently selected from D, F, Cl, Br, I, OH, NH₂, CN, C₁₋₃ alkoxy, and —C(═O)—N(R_{b1}) (R_{b2});
R₈ is selected from H, F, Cl, Br, I, OH, NH₂, CN, C₁₋₃ alkyl, and C₁₋₃ alkoxy, wherein the C₁₋₃ alkyl and C₁₋₃ alkoxy are each independently optionally substituted with 1, 2, 3, 4, or 5 Rₐ,
or R₈ and R_{8′} together with the carbon atoms to which they are attached form a C₃₋₅ cycloalkyl or 3-5 membered heterocyclyl; wherein the C₃₋₅ cycloalkyl or 3-5 membered heterocyclyl is each independently optionally substituted with 1, 2 or 3 R₁₀;
R₉ is —C(═O)—NR_{b1}R_{b2} or —CH₂R_{c};
each R₁₀ is independently selected from oxo, D, F, Cl, Br, I, OH, NH₂, CN, C₁₋₃ alkyl, C₁₋₃ alkoxy, C₁₋₃ alkylamino, diC₁₋₃ alkylamino, —S—R_{d}, —S(═O)—R_{d}, —S(═O)₂—R_{d}, and —NH—C(═O)—R_{d}; wherein the C₁₋₃ alkyl is optionally substituted with 1, 2, or 3 OH;
R_{b1} and R_{b2} are each independently selected from H, C₁₋₆ alkyl, C₃₋₁₀ cycloalkyl, 3-10 membered heterocyclyl, C₆₋₁₀ aryl, and 5-10 membered heteroaryl; wherein the C₁₋₆ alkyl, C₃₋₁₀ cycloalkyl, 3-10 membered heterocyclyl, C₆₋₁₀ aryl, and 5-10 membered heteroaryl are each independently optionally substituted with 1, 2, or 3 Rₑ;
or, R_{b1} and R_{b2} together with the nitrogen atom to which they are attached form a 3-6 membered heterocyclyl;
R_{c} is selected from F, Cl, Br, I, OH, NH₂, —O(C═O)NR_{CI}R_{C2}, —NR_{C0}(C═O)R_{C1}, and —NR_{C0}(C═O)NR_{C1}R_{C2};
R_{C0}, R_{C1}, and R_{C2} are each independently selected from H, C₁₋₆ alkyl, C₃₋₆ cycloalkyl, and 3-6 membered heterocyclyl;
R_{d} is C₁₋₃ alkyl;
Rₑ is selected from the group consisting of F, Cl, Br, I, OH, NH₂, C₁₋₃ alkylamino, diC₁₋₃ alkylamino, CN, C₁₋₃ alkoxy, C₃₋₁₀ cycloalkyl, 3-10 membered heterocyclyl, C₆₋₁₀ aryl, and 5-10 membered heteroaryl;
m is 0, 1, 2, 3, 4, or 5;
provided that,
1) when Ring B is

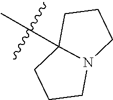 [SMILES: *C12CCCN1CCC2]

optionally substituted with one R₁₀ and when R₁₀ is F, at least one of R₁, R₂, R₄, R₅, R₆, R₇, R_{6′} and R_{7′} is not H;
2) when Ring B is

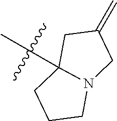 [SMILES: *C12CCCN1CC(=C)C2]

optionally substituted with 1, 2, 3, or 4 R₁₀, at least one of R₁, R₂, R₄, R₅, R₆, R₇, R_{6′} and R_{7′} is not H; and
3) the compound is not

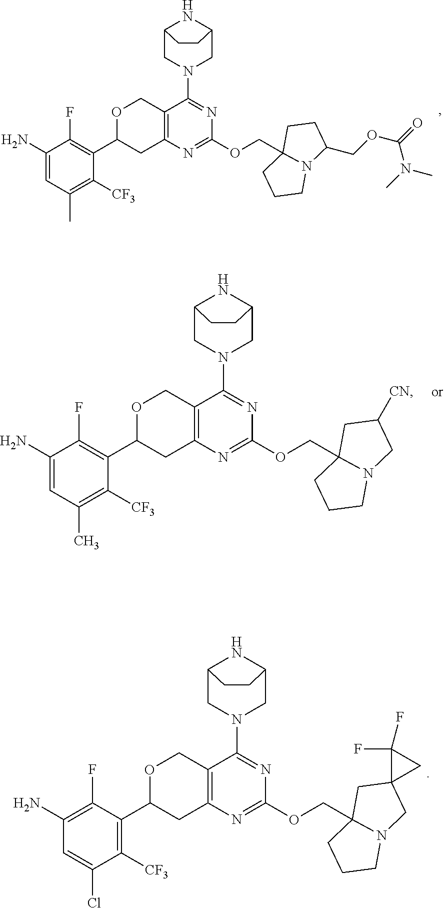 [SMILES: Cc1cc(N)c(F)c(C2Cc3nc(OCC45CCCN4C(COC(=O)N(C)C)CC5)nc(N4CC5CCC(C4)N5)c3CO2)c1C(F)(F)F ; Cc1cc(N)c(F)c(C2Cc3nc(OCC45CCCN4CC(C#N)C5)nc(N4CC5CCC(C4)N5)c3CO2)c1C(F)(F)F ; Nc1cc(Cl)c(C(F)(F)F)c(C2Cc3nc(OCC45CCCN4CC4(C5)CC4(F)F)nc(N4CC5CCC(C4)N5)c3CO2)c1F]

In some embodiments, the present disclosure provides a compound of Formula (I′-1):

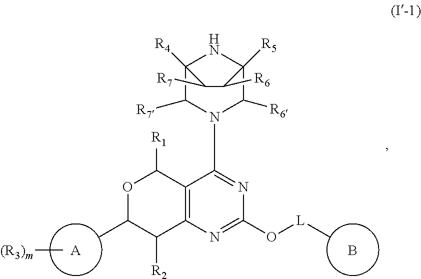

a stereoisomer thereof, or a pharmaceutically acceptable salt thereof, wherein:
Ring A is C₆ aryl;
Ring B is selected from

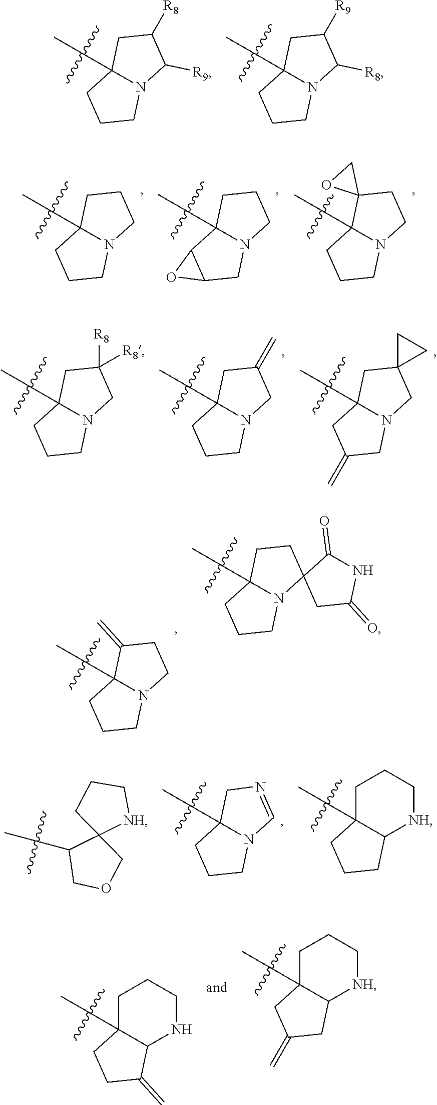

wherein Ring B is optionally substituted with 1, 2, 3, or 4 R₁₀;
L is —CH₂-optionally substituted with 1 or 2 D;
R₁ and R₂ are each independently selected from oxo, H, F, Cl, Br, I, and CN;
each R₃ is independently selected from F, Cl, Br, I, OH, NH₂, CN, C₁₋₃ alkyl, C₁₋₃ alkoxy, C₁₋₃ alkylamino, diC₁₋₃ alkylamino, C₂₋₄ alkenyl, C₂₋₄ alkynyl, and C₃₋₅ cycloalkyl, wherein the C₁₋₃ alkyl, C₁₋₃ alkoxy, C₁₋₃ alkylamino, diC₁₋₃alkylamino, C₂₋₄alkenyl, C₂₋₄ alkynyl and C₃₋₅ cycloalkyl are each independently substituted with 1, 2, 3, 4, or 5 Rₐ;
each Rₐ is independently selected from D, F, Cl, Br, and I;
R₄, R₅, R₆, R₇, R_{6′}, and R_{7′} are each independently selected from oxo, H, F, Cl, Br, I, OH, NH₂, CN, C₁₋₃ alkyl, C₂₋₄ alkenyl, C₁₋₃ alkoxy, —C(═O)—R_{d}, —C(═O)—NR_{b1}R_{b2}, and =NO(C₁₋₃ alkyl), wherein the C₁₋₃ alkyl, C₂₋₄ alkenyl, and C₁₋₃ alkoxy are each independently optionally substituted with 1, 2, 3, 4, or 5 R_{b};
or, R₆ and R₇ together with the carbon atoms to which they are attached form a 3-5 membered heterocyclyl;
each R_{b} is independently selected from D, F, Cl, Br, I, OH, NH₂, CN, C₁₋₃ alkoxy, and —C(═O)—N(R_{b1})(R_{b2});
R₈ is selected from the group consisting of H, F, Cl, Br, I, OH, NH₂, CN, C₁₋₃ alkyl, and C₁₋₃ alkoxy, wherein the C₁₋₃ alkyl and C₁₋₃ alkoxy are each independently optionally substituted with 1, 2, 3, 4, or 5 Rₐ;
or, R₈ and R_{8′} together with the carbon atoms to which they are attached form a C₃₋₅ cycloalkyl or 3-5 membered heterocyclyl; wherein the C₃₋₅ cycloalkyl and 3-5 membered heterocyclyl are each independently optionally substituted with 1, 2 or 3 R₁₀;
R₉ is —C(═O)—NR_{b1}R_{b2} or —CH₂R_{c},
each R₁₀ is independently selected from oxo, D, F, Cl, Br, I, OH, NH₂, CN, C₁₋₃ alkyl, C₁₋₃ alkoxy, C₁₋₃ alkylamino, diC₁₋₃ alkylamino, —S—R_{d}, —S(═O)—R_{d}, —S(═O)₂—R_{d}, and —NH—C(═O)—R_{d}; wherein the C₁₋₃ alkyl is optionally substituted with 1, 2, or 3 OH;
R_{b1} and R_{b2} are each independently selected from H, C₁₋₆ alkyl, C₃₋₁₀ cycloalkyl, 3-10 membered heterocyclyl, C₆₋₁₀ aryl, and 5-10 membered heteroaryl; wherein the C₁₋₆ alkyl, C₃₋₁₀ cycloalkyl, 3-10 membered heterocyclyl, C₆₋₁₀ aryl, and 5-10 membered heteroaryl are each independently optionally substituted with 1, 2, or 3 R_{c};
or, R_{b1} and R_{b2} together with the nitrogen atom to which they are attached form a 3-6 membered heterocyclyl;
R_{c} is selected from F, Cl, Br, I, OH, NH₂, —O(C═O)NR_{C1}R_{C2}, —NR_{C0}(C═O)R_{C1}, and —NR_{C0}(C═O)NR_{C1}R_{C2};
R_{C0}, R_{C1}, and R_{C2} are each independently selected from H, C₁₋₆ alkyl, C₃₋₆ cycloalkyl, and 3-6 membered heterocyclyl;
R_{d} is C₁₋₃ alkyl;
Rₑ is selected from F, Cl, Br, I, OH, NH₂, C₁₋₃ alkylamino, diC₁₋₃ alkylamino, CN, C₁₋₃ alkoxy, C₃₋₁₀ cycloalkyl, 3-10 membered heterocyclyl, C₆₋₁₀ aryl, and 5-10 membered heteroaryl;
m is 0, 1, 2, 3, 4, or 5;
provided that,
1) when Ring B is

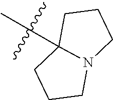 [SMILES: *C12CCCN1CCC2]

optionally substituted with one R₁₀ and when R₁₀ is F, at least one of R₁, R₂, R₄, R₅, R₆, R₇, R_{6′} and R_{7′} is not H;
2) when Ring B is

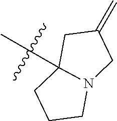 [SMILES: *C12CCCN1CC(=C)C2]

optionally substituted with 1, 2, 3 or 4 R₁₀, at least one of R₁, R₂, R₄, R₅, R₆, R₇, R_{6′} and R_{7′} is not H; and
3) the compound is not

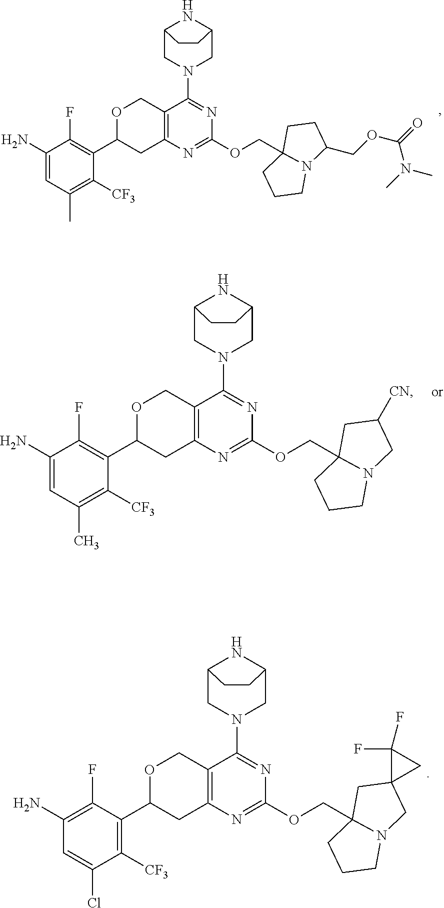 [SMILES: Cc1cc(N)c(F)c(C2Cc3nc(OCC45CCCN4C(COC(=O)N(C)C)CC5)nc(N4CC5CCC(C4)N5)c3CO2)c1C(F)(F)F ; Cc1cc(N)c(F)c(C2Cc3nc(OCC45CCCN4CC(C#N)C5)nc(N4CC5CCC(C4)N5)c3CO2)c1C(F)(F)F ; Nc1cc(Cl)c(C(F)(F)F)c(C2Cc3nc(OCC45CCCN4CC4(C5)CC4(F)F)nc(N4CC5CCC(C4)N5)c3CO2)c1F]

In some embodiments, the present disclosure provides a compound of Formula (I′-2):

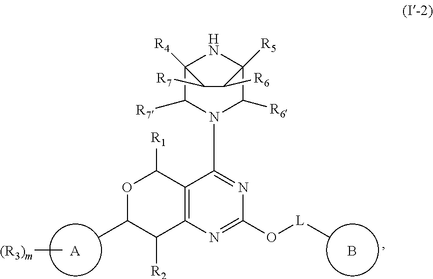

a stereoisomer thereof, or a pharmaceutically acceptable salt thereof, wherein:
Ring A is 5-membered heteroaryl;
Ring B is selected from

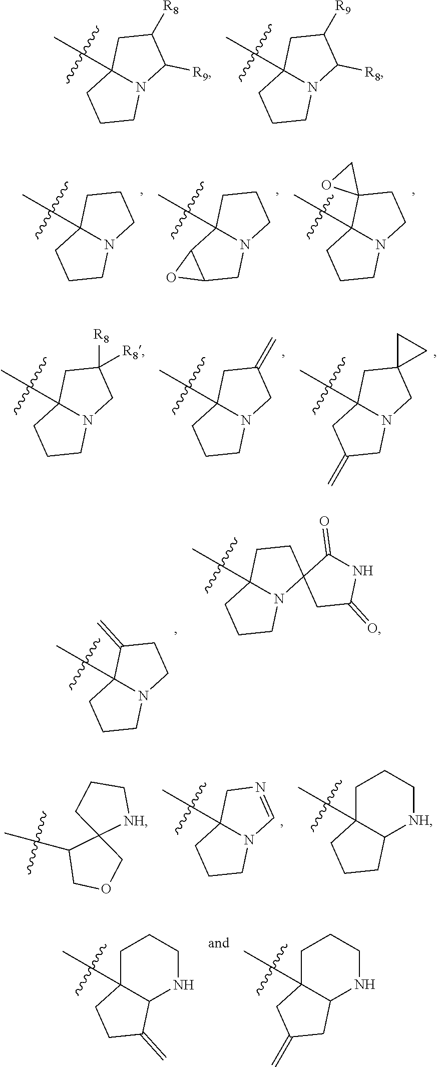

wherein Ring B is optionally substituted with 1, 2, 3, or 4 R₁₀;
L is —CH₂-optionally substituted with 1 or 2 D;
R₁ and R₂ are each independently selected from oxo, H, F, Cl, Br, I, and CN;
each R₃ is independently selected from F, Cl, Br, I, OH, NH₂, CN, C₁₋₃ alkyl, C₁₋₃ alkoxy, C₁₋₃ alkylamino, diC₁₋₃ alkylamino, C₂₋₄ alkenyl, C₂₋₄ alkynyl, and C₃₋₅ cycloalkyl, wherein the C₁₋₃ alkyl, C₁₋₃ alkoxy, C₁₋₃ alkylamino, diC₁₋₃alkylamino, C₂₋₄alkenyl, C₂₋₄ alkynyl, and C₃₋₅ cycloalkyl are each independently substituted with 1, 2, 3, 4, or 5 Rₐ;
each Rₐ is independently selected from D, F, Cl, Br, and I;
R₄, R₅, R₆, R₇, R_{6′}, and R_{7′} are each independently selected from oxo, H, F, Cl, Br, I, OH, NH₂, CN, C₁₋₃ alkyl, C₂₋₄ alkenyl, C₁₋₃ alkoxy, —C(═O)—R_{d}, —C(═O)—NR_{b1}R_{b2}, and ═NO(C₁₋₃ alkyl), wherein the C₁₋₃ alkyl, C₂₋₄ alkenyl, and C₁₋₃ alkoxy are each independently optionally substituted with 1, 2, 3, 4, or 5 R_{b};
or, R₆ and R₇ together with the carbon atoms to which they are attached form a 3-5 membered heterocyclyl;
each R_{b} is independently selected from D, F, Cl, Br, I, OH, NH₂, CN, C₁₋₃ alkoxy, and —C(═O)—N(R_{b1})(R_{b2});
R₈ is selected from the group consisting of H, F, Cl, Br, I, OH, NH₂, CN, C₁₋₃ alkyl, and C₁₋₃ alkoxy, wherein the C₁₋₃ alkyl and C₁₋₃ alkoxy are each independently optionally substituted with 1, 2, 3, 4, or 5 Rₐ;
or, R₈ and R_{8′} together with the carbon atoms to which they are attached form a C₃₋₅ cycloalkyl or 3-5 membered heterocyclyl; wherein the C₃₋₅ cycloalkyl and 3-5 membered heterocyclyl are each independently optionally substituted with 1, 2 or 3 R₁₀,
R₉ is selected from —C(═O)—NR_{b1}R_{b2} and —CH₂R_{c};
each R₁₀ is independently selected from oxo, D, F, Cl, Br, I, OH, NH₂, CN, C₁₋₃ alkyl, C₁₋₃ alkoxy, C₁₋₃ alkylamino, diC₁₋₃ alkylamino, —S—R_{d}, —S(═O)—R_{d}, —S(═O)₂—R_{d}, and —NH—C(═O)—Rₐ; wherein the C₁₋₃ alkyl is optionally substituted with 1, 2, or 3 OH;
R_{b1} and R_{b2} are each independently selected from H, C₁₋₆ alkyl, C₃₋₁₀ cycloalkyl, 3-10 membered heterocyclyl, C₆₋₁₀ aryl, and 5-10 membered heteroaryl; wherein the C₁₋₆ alkyl, C₃₋₁₀ cycloalkyl, 3-10 membered heterocyclyl, C₆₋₁₀ aryl, and 5-10 membered heteroaryl are each independently optionally substituted with 1, 2, or 3 R_{c};
or, R_{b1} and R_{b2} together with the nitrogen atom to which they are attached form a 3-6 membered heterocyclyl;
R_{c} is selected from F, Cl, Br, I, OH, NH₂, —O(C═O)NR_{C1}R_{C2}, —NR_{C0}(C═O)R_{C1}, and —NR_{C0}(C═O)NR_{C1}R_{C2};
R_{C0}, R_{C1}, and R_{C2} are each independently selected from H, C₁₋₆ alkyl, C₃₋₆ cycloalkyl, and 3-6 membered heterocyclyl;
R_{d} is C₁₋₃ alkyl;
Rₑ is selected from F, Cl, Br, I, OH, NH₂, C₁₋₃ alkylamino, di-C₁₋₃ alkylamino, CN, C₁₋₃ alkoxy, C₃₋₁₀ cycloalkyl, 3-10 membered heterocyclyl, C₆₋₁₀ aryl, and 5-10 membered heteroaryl;
m is selected from 0, 1, 2, 3, 4 and 5;
provided that,
1) when Ring B is

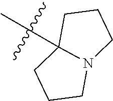 [SMILES: *C12CCCN1CCC2]

optionally substituted with one R₁₀ and when R₁₀ is F, at least one of R₁, R₂, R₄, R₅, R₆, R₇, R_{6′} and R_{7′} is not H;
2) when Ring B is

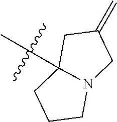 [SMILES: *C12CCCN1CC(=C)C2]

optionally substituted with 1, 2, 3 or 4 R₁₀, at least one of R₁, R₂, R₄, R₅, R₆, R₇, R_{6′} and R_{7′} is not H; and
3) the compound is not

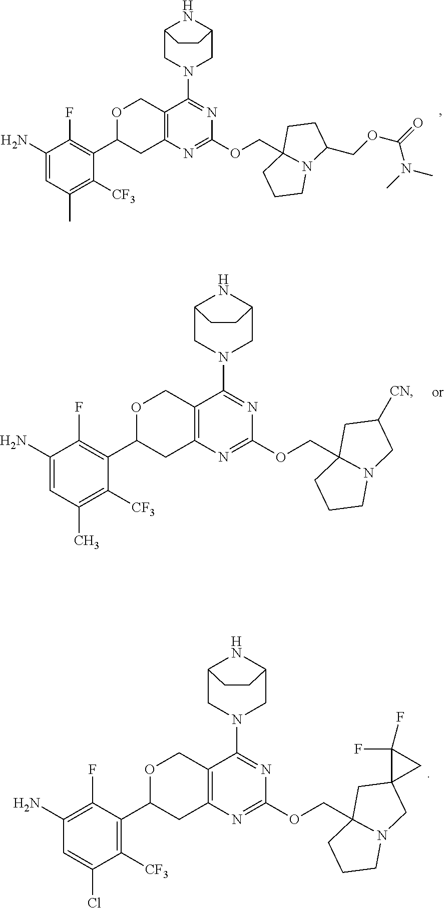 [SMILES: Cc1cc(N)c(F)c(C2Cc3nc(OCC45CCCN4C(COC(=O)N(C)C)CC5)nc(N4CC5CCC(C4)N5)c3CO2)c1C(F)(F)F ; Cc1cc(N)c(F)c(C2Cc3nc(OCC45CCCN4CC(C#N)C5)nc(N4CC5CCC(C4)N5)c3CO2)c1C(F)(F)F ; Nc1cc(Cl)c(C(F)(F)F)c(C2Cc3nc(OCC45CCCN4CC4(C5)CC4(F)F)nc(N4CC5CCC(C4)N5)c3CO2)c1F]

In some embodiments, Ring B is selected from

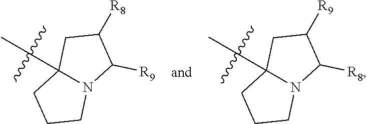

wherein Ring B is optionally substituted with 1, 2, 3, or 4 R₁₀.

In some embodiments, ring B is

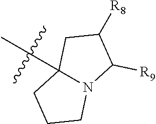

optionally substituted with 1, 2, 3, or 4 R₁₀. In some embodiments, Ring B is unsubstituted. In some embodiments, Ring B is substituted. In some embodiments, Ring B is substituted with 1 or 2 R₁₀. In some embodiments, Ring B is substituted with 1 R₁₀.

In any of the embodiments disclosed herein as applicable, Ring B is substituted with 2 R₁₀.

In some embodiments, the compound of Formula (I′-1), a stereoisomer thereof, or a pharmaceutically acceptable salt thereof, is a compound of Formula (I′-1-j), a compound of Formula (I′-2-i),

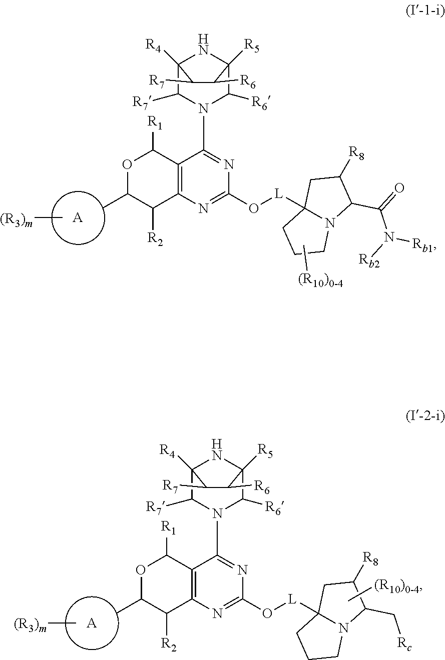

a stereoisomer thereof, or a pharmaceutically acceptable salt thereof.

In some embodiments, the compound of Formula (I′-1), a stereoisomer thereof, or a pharmaceutically acceptable salt thereof is a compound of Formula (I′-1-i), a stereoisomer thereof, or a pharmaceutically acceptable salt thereof.

In some embodiments, the compound of Formula (I′-1), a stereoisomer thereof, or a pharmaceutically acceptable salt thereof is a compound of Formula (I′-2-i), a stereoisomer thereof, or a pharmaceutically acceptable salt thereof.

In some embodiments, Ring B is

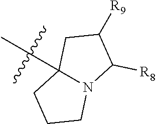

optionally substituted with 1, 2, 3, or 4 R₁₀. In some embodiments, Ring B is unsubstituted. In some embodiments, Ring B is substituted with 1 or 2 R₁₀. In some embodiments, Ring B is substituted with 1 R₁₀. In some embodiments, Ring B is substituted with 2 R₁₀.

In some embodiments, the compound of Formula (I′-1), a stereoisomer thereof, or a pharmaceutically acceptable salt thereof is a compound of Formula (l′-1-ii), a compound of Formula (I′-2-ii),

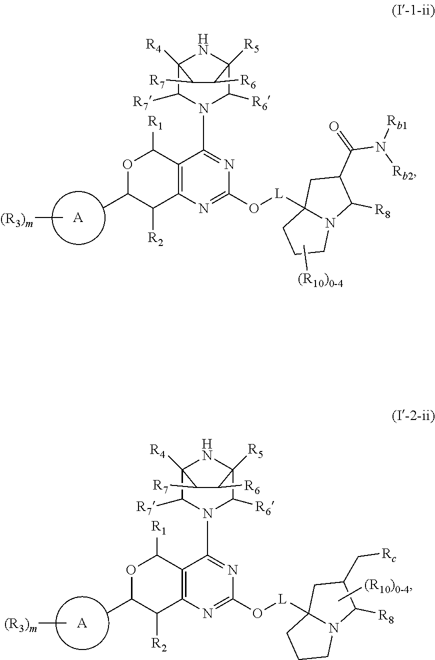

a stereoisomer thereof, or a pharmaceutically acceptable salt thereof.

In some embodiments, the compound of Formula (I′-1), a stereoisomer thereof, or a pharmaceutically acceptable salt thereof is a compound of Formula (I′-1-ii), a stereoisomer thereof, or a pharmaceutically acceptable salt thereof.

In some embodiments, the compound of Formula (I′-1), a stereoisomer thereof, or a pharmaceutically acceptable salt thereof is a compound of Formula (I′-2-ii), a stereoisomer thereof, or a pharmaceutically acceptable salt thereof.

In some embodiments, Ring B is selected from

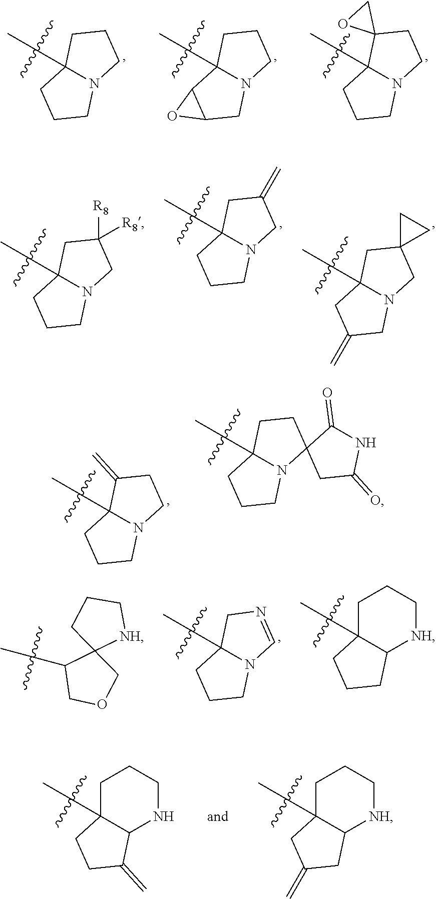

wherein Ring B is optionally substituted with 1, 2, 3, or 4 R₁₀. In some embodiments, Ring B is unsubstituted. In some embodiments, Ring B is substituted with 1 or 2 R₁₀. In some embodiments, Ring B is substituted with 1 R₁₀. In some embodiments, Ring B is substituted with 2 R₁₀.

In some embodiments, the compound of Formula (I′-1), a stereoisomer thereof, or a pharmaceutically acceptable salt thereof, is a compound of Formulae (I′-3), (I′-4), (I′-5), (I′-6), (I′-7), (I′-8), (I′-9), (I′-10), (I′-11), (I′-12), or (I′-13).

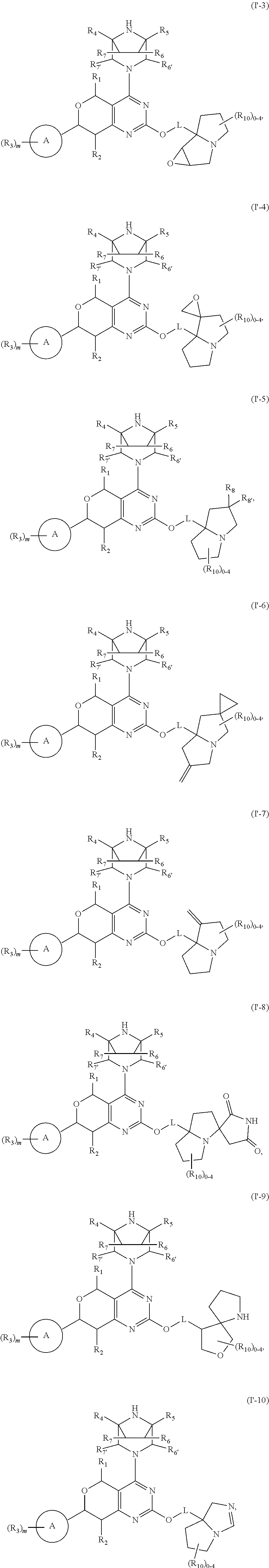

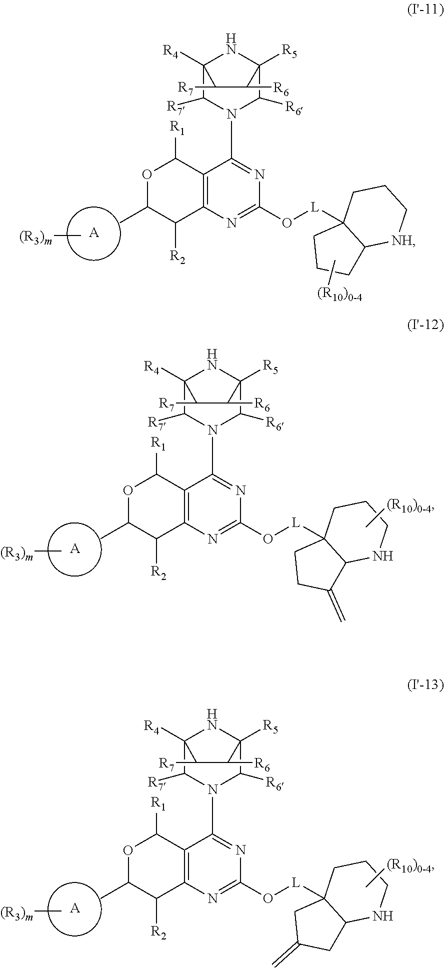

a stereoisomer thereof, or a pharmaceutically acceptable salt thereof.

In some embodiments, the compound of Formula (I′-1), a stereoisomer thereof, or a pharmaceutically acceptable salt thereof, is a compound of Formula (I′-14), a compound of Formula (I′-15),

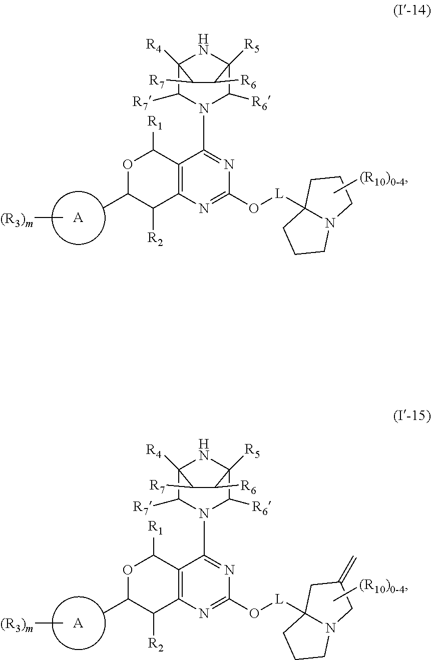

a stereoisomer thereof, or a pharmaceutically acceptable salt thereof.

In any of the embodiments disclosed herein as applicable, Ring A is phenyl substituted with at least 1 R₃; wherein each R₃ is independently selected from F, Cl, Br, I, OH, NH₂, CN, C₁₋₃ alkyl, C₁₋₃ alkoxy, C₁₋₃ alkylamino, C₂₋₄ alkenyl, C₂₋₄ alkynyl, and C₃₋₅ cycloalkyl, wherein the C₁₋₃ alkyl, C₁₋₃ alkoxy, C₁₋₃ alkylamino, C₂₋₄ alkenyl, C₂₋₄ alkynyl, and C₃₋₅ cycloalkyl are each independently substituted with 1, 2, 3, 4, or 5 Rₐ.

In any of the embodiments disclosed herein as applicable, Ring A is a 5-membered heteroaryl substituted with at least 1 R₃; wherein each R₃ is independently selected from F, OH, NH₂, CF₃, OCH₃, —C≡CCH₃, —C≡CCH₂F, —C≡CCHF₂, —C≡CCF₃, —C≡CCD₃, —C≡CH, —C≡CCl, —C≡CF and cyclopropyl.

In some embodiments, Ring A is a 5-membered heteroaryl substituted with 2 R₃; wherein each R₃ is independently selected from F, OH, NH₂, CF, OCH₃, —C≡CCH₃, —C≡CCH₂F, —C≡CCHF₂, —C≡CCF₃, —C≡CCD₃, —C≡CH, —C≡CCl, —C≡CF and cyclopropyl.

In any of the embodiments disclosed herein as applicable, Ring A is a 5-membered heteroaryl substituted with 3 R₃; wherein each R₃ is independently selected from F, OH, NH₂, CF₃, OCH₃, —C≡CCH₃, —C≡CCH₂F, —C≡CCHF₂, —C≡CCF₃, —C≡CCD₃, —C≡CH, —C≡CCl, —C≡CF and cyclopropyl.

In any of the embodiments disclosed herein as applicable, Ring A is a 5-membered heteroaryl substituted with 4 R₃; wherein each R₃ is independently selected from F, OH, NH₂, CF₃, OCH₃, —C≡CCH₃, —C≡CCH₂F, —C≡CCHF₂, —C≡CCF₃, —C≡CCD₃, —C≡CH, —C≡CCl, —C≡CF and cyclopropyl.

In some embodiments,

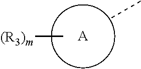

is selected from

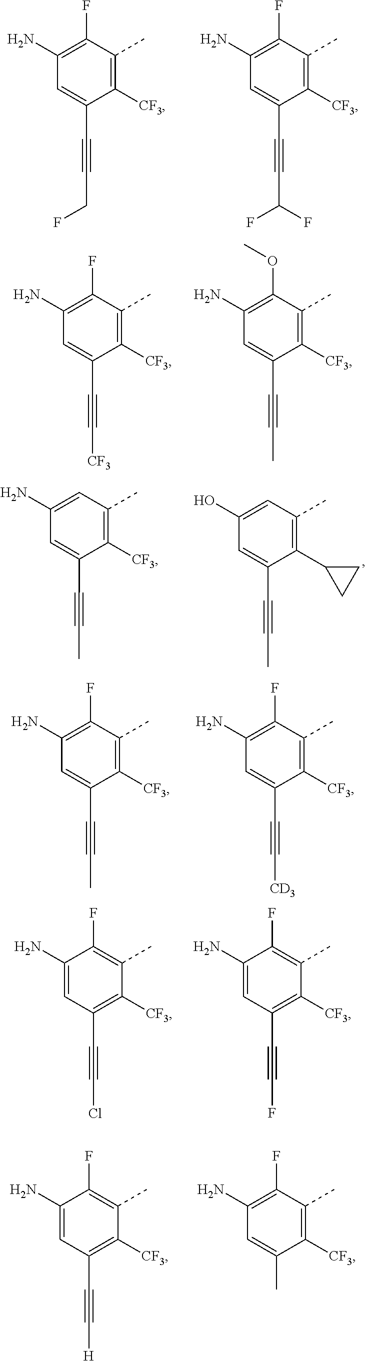

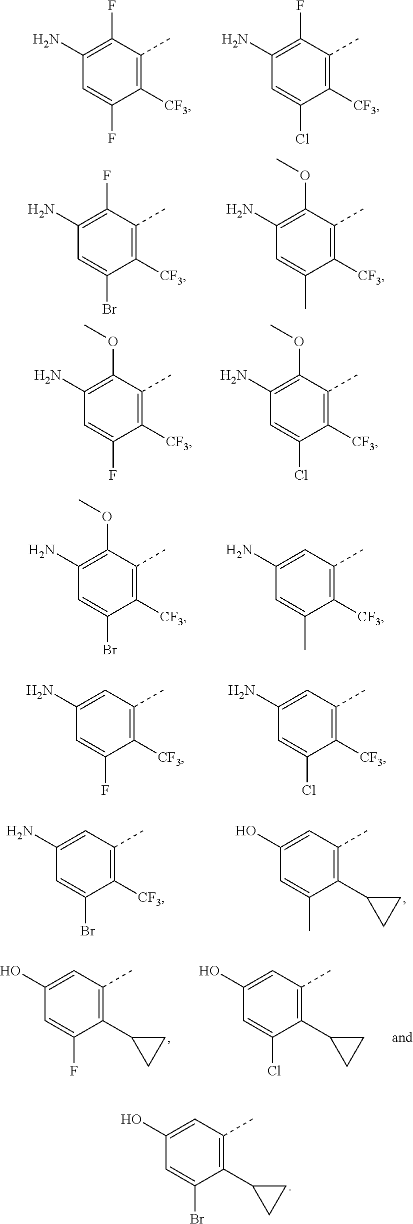

In some embodiments,

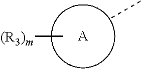

is selected from

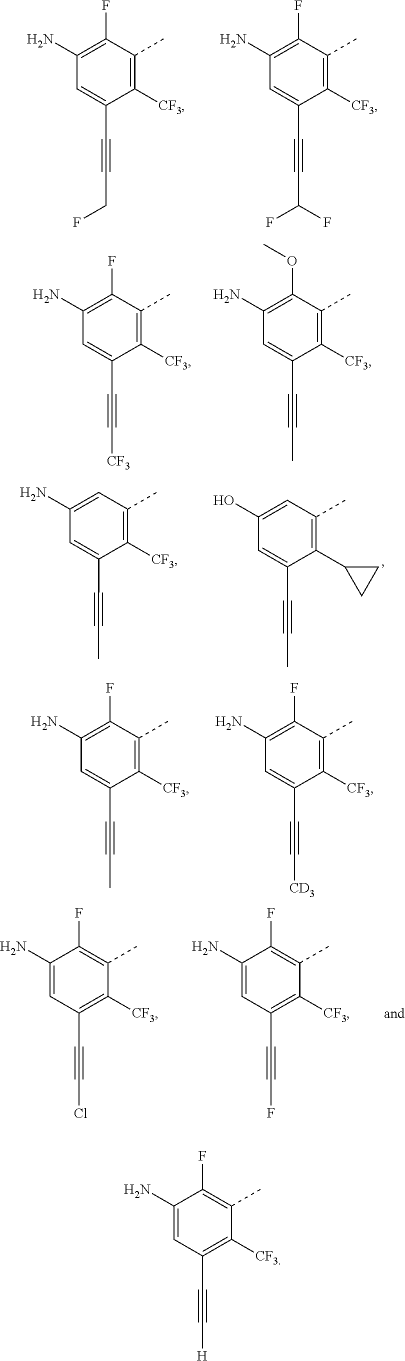

In any of the embodiments disclosed herein as applicable,

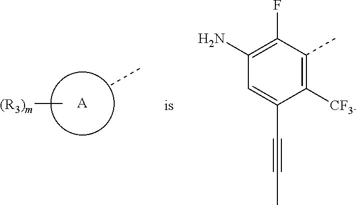

In some embodiments,

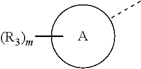

is selected from

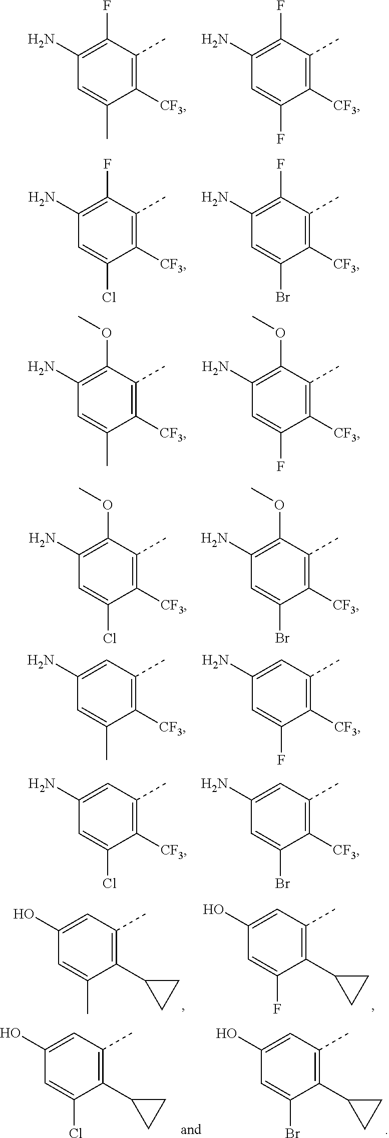

In any of the embodiments disclosed herein as applicable, Ring A is a 5-6 membered heteroaryl (e.g. pyridinyl) substituted with at least 1 R₃; each R₃ is independently selected from F, Cl, Br, I, OH, NH₂, CN, C₁₋₃ alkyl, C₁₋₃ alkoxy, C₁₋₃ alkylamino, C₂₋₄ alkenyl, C₂₋₄ alkynyl, and C₃₋₅ cycloalkyl, wherein the C₁₋₃ alkyl, C₁₋₃ alkoxy, C₁₋₃ alkylamino, C₂₋₄ alkenyl, C₂₋₄ alkynyl, and C₃₋₅ cycloalkyl are each independently substituted with 1, 2, 3, 4, or 6 Rₐ.

In any of the embodiments disclosed herein as applicable, Ring A is a 5-6 membered heteroaryl (e.g., pyridyl) substituted with at least 1 R₃, each R₃ being independently selected from F, OH, NH₂, CF₃, OCH₃, —C≡CCH₃, —C≡CCH₂F, —C≡CCHF₂, —C≡CCF₃, —C≡CCD₃, —C≡CH, —C≡CCl, —C≡CF and cyclopropyl.

In any of the embodiments disclosed herein as applicable, Ring A is a 5-6 membered heteroaryl (e.g., pyridyl) substituted with 2 R₃, each R₃ is independently selected from F, OH, NH₂, CF₃, OCH₃, —C≡CCH₃, —C≡CCH₂F, —C≡CCHF₂, —C≡CCF₃, —C≡CCD₃, —C≡CH, —C≡CCl, —C≡CF and cyclopropyl.

In any of the embodiments disclosed herein as applicable, ring A is a 5-6 membered heteroaryl (e.g., pyridyl) substituted with 3 R₃, each R₃ is independently selected from F, OH, NH₂, CF₃, OCH₃, —C≡CCH₃, —C≡CCH₂F, —C≡CCHF₂, —C≡CCF₃, —C≡CCD₃, —C≡CH, —C≡CCl, —C≡CF and cyclopropyl. In some embodiments, ring A is selected from 5-6 membered heteroaryl (e.g., pyridyl) substituted with 4 R₃, each R₃ is independently selected from F, OH, NH₂, CF₃, OCH₃, —C≡CCH₃, —C≡CCH₂F, —C≡CCHF₂, —C≡CCF₃, —C≡CCD₃, —C≡CH, —C≡CCl, —C≡CF and cyclopropyl.

In any of the embodiments disclosed herein as applicable, ring A is a 5-membered heteroaryl, substituted with at least 1 R₃; each R₃ is independently selected from F, Cl, Br, I, OH, NH₂, CN, C₁₋₃ alkyl, C₁₋₃ alkoxy, C₁₋₃ alkylamino, C₂₋₄ alkenyl, C₂₋₄ alkynyl, and C₃₋₅ cycloalkyl, wherein the C₁₋₃ alkyl, C₁₋₃ alkoxy, C₁₋₃ alkylamino, C₂₋₄ alkenyl, C₂₋₄ alkynyl, and C₃₋₅ cycloalkyl are each independently substituted with 1, 2, 3, 4, or 5 Rₐ.

In any of the embodiments disclosed herein as applicable, ring A is a 5-membered heteroaryl substituted with at least 1 R₃; each R₃ is independently selected from F, OH, NH₂, CF₃, OCH₃, —C≡CCH₃, —C≡CCH₂F, —C≡CCHF₂, —C≡CCF₃, —C≡CCD₃, —C≡CH, —C≡CCl, —C≡CF and cyclopropyl.

In any of the embodiments disclosed herein as applicable, ring A is a 5-membered heteroaryl substituted with at least 2 R₃; each R₃ is independently selected from F, OH, NH₂, CF₃, OCH₃, —C≡CCH₃, —C≡CCH₂F, —C≡CCHF₂, —C≡CCF₃, —C≡CCD₃, —C≡CH, —C≡CCl, —C≡CF and cyclopropyl.

In any of the embodiments disclosed herein as applicable, ring A is a 5-membered heteroaryl substituted with at least 3 R₃; each R₃ is independently selected from F, OH, NH₂, CF₃, OCH₃, —C≡CCH₃, —C≡CCH₂F, —C≡CCHF₂, —C≡CCF₃, —C≡CCD₃, —C≡CH, —C≡CCl, —C≡CF and cyclopropyl.

In any of the embodiments disclosed herein as applicable, ring A is a 5-membered heteroaryl substituted with at least 4 R₃; each R₃ is independently selected from F, OH, NH₂, CF₃, OCH₃, —C≡CCH₃, —C≡CCH₂F, —C≡CCHF₂, —C≡CCF₃, —C≡CCD₃, —C≡CH, —C≡CCl, —C≡CF and cyclopropyl.

In any of the embodiments disclosed herein as applicable, ring A is a 6-membered heteroaryl substituted with at least 1 R₃; each R₃ is independently selected from F, Cl, Br, I, OH, NH₂, CN, C₁₋₃ alkyl, C₁₋₃ alkoxy, C₁₋₃ alkylamino, C₂₋₄ alkenyl, C₂₋₄ alkynyl, and C₃₋₅ cycloalkyl, wherein the C₁₋₃ alkyl, C₁₋₃ alkoxy, C₁₋₃ alkylamino, C₂₋₄ alkenyl, C₂₋₄ alkynyl, and C₃₋₅ cycloalkyl is independently substituted with 1, 2, 3, 4, or 6 R₃.

In any of the embodiments disclosed herein as applicable, ring A is a 6-membered heteroaryl substituted with at least 1 R₃; each R₃ is independently selected from F, OH, NH₂, CF₃, OCH₃, —C≡CCH₃, —C≡CCH₂F, —C≡CCHF₂, —C≡CCF₃, —C≡CCD₃, —C≡CH, —C≡CCl, —C≡CF and cyclopropyl.

In any of the embodiments disclosed herein as applicable, ring A is a 6-membered heteroaryl substituted with at least 2 R₃; each R₃ is independently selected from F, OH, NH₂, CF₃, OCH₃, —C≡CCH₃, —C≡CCH₂F, —C≡CCHF₂, —C≡CCF₃, —C≡CCD₃, —C≡CH, —C≡CCl, —C≡CF and cyclopropyl.

In any of the embodiments disclosed herein as applicable, ring A is a 6-membered heteroaryl substituted with at least 3 R₃; each R₃ is independently selected from F, OH, NH₂, CF₃, OCH₃, —C≡CCH₃, —C≡CCH₂F, —C≡CCHF₂, —C≡CCF₃, —C≡CCD₃, —C≡CH, —C≡CCl, —C≡CF and cyclopropyl.

In any of the embodiments disclosed herein as applicable, ring A is a 6-membered heteroaryl substituted with at least 4 R₃; each R₃ is independently selected from F, OH, NH₂, CF₃, OCH₃, —C≡CCH₃, —C≡CCH₂F, —C≡CCHF₂, —C≡CCF₃, —C≡CCD₃, —C≡CH, —C≡CCl, —C≡CF and cyclopropyl.

In some embodiments, the compound of Formula (I′-1), a stereoisomer thereof, or a pharmaceutically acceptable salt thereof, is selected from a compound of Formula (I′-1′-i), a compound of Formula (l′-2′-i),

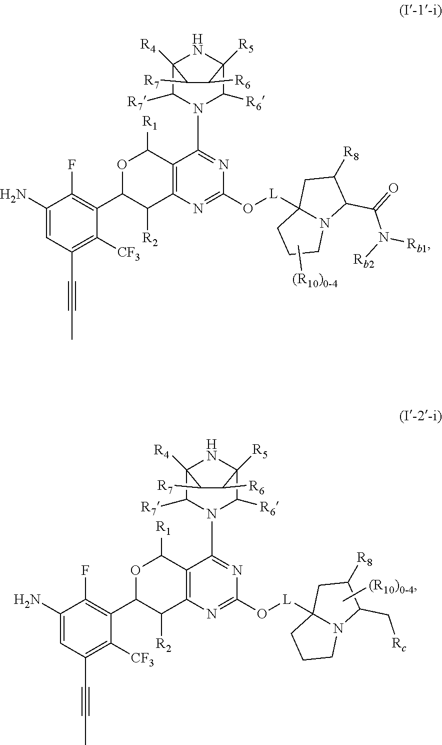

a stereoisomer thereof, or a pharmaceutically acceptable salt thereof.

In any of the embodiments disclosed herein as applicable, the compound of Formula (I′-1), a stereoisomer thereof, or a pharmaceutically acceptable salt thereof is a compound of Formula (I′-1′-i), a stereoisomer thereof, or a pharmaceutically acceptable salt thereof.

In any of the embodiments disclosed herein as applicable, the compound of Formula (I′-1), a stereoisomer thereof, or a pharmaceutically acceptable salt thereof is a compound of Formula (I′-2′-i), a stereoisomer thereof, or a pharmaceutically acceptable salt thereof.

In some embodiments, the compound of Formula (I′-1), a stereoisomer thereof, or a pharmaceutically acceptable salt thereof is a compound of Formula (I′-1′-ii), a compound of Formula (I′-2′-ii),

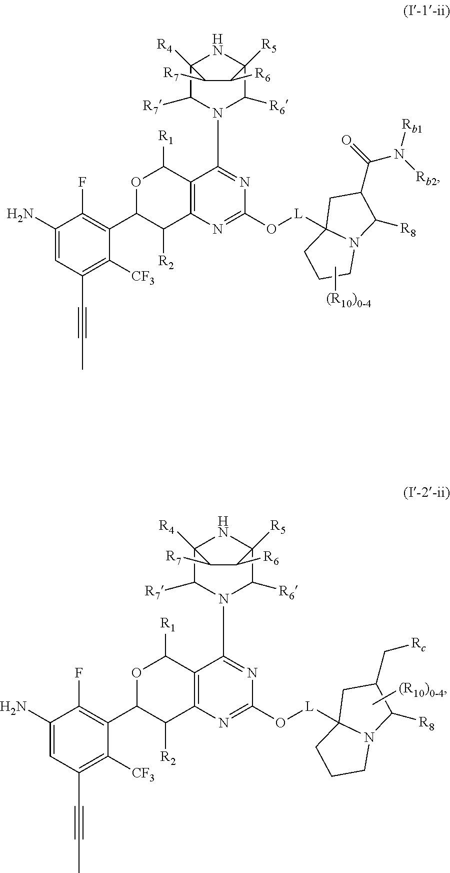

a stereoisomer thereof, or a pharmaceutically acceptable salt thereof.

In any of the embodiments disclosed herein as applicable, the compound of Formula (I′-1), a stereoisomer thereof, or a pharmaceutically acceptable salt thereof is a compound of Formula (I′-1′-ii), a stereoisomer thereof, or a pharmaceutically acceptable salt thereof.

In any of the embodiments disclosed herein as applicable, the compound of Formula (I′-1), a stereoisomer thereof, or a pharmaceutically acceptable salt thereof is a compound of Formula (I′-2′-ii), a stereoisomer thereof, or a pharmaceutically acceptable salt thereof.

In some embodiments, the compound of Formula (I′-1), a stereoisomer thereof, or a pharmaceutically acceptable salt thereof is selected from a compound of Formulae (I′-3′), (I′-4′), (I′-5′), (I′-6′), (I′-7′), (I′-8′), (I′-9′), (I′-10′), (I′-11′), (I′-12′), (I′-13′),

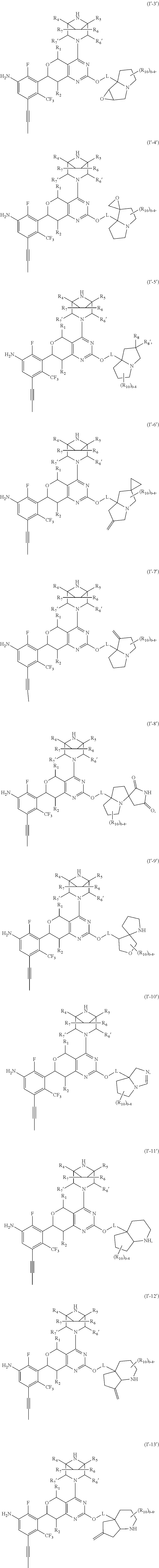

a stereoisomer thereof, or a pharmaceutically acceptable salt thereof.

In some embodiments, the compound of Formula (I′-1), or a stereoisomer thereof, or a pharmaceutically acceptable salt thereof is a compound of Formula (I′-14′), a compound of Formula (I′-15′),

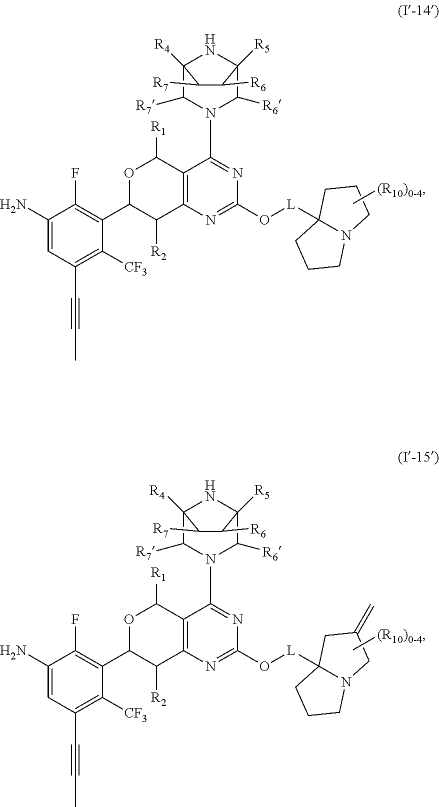

a stereoisomer thereof, or a pharmaceutically acceptable salt thereof.

In some embodiments, Ring A is selected from a C₆ aryl and a 5-6 membered heteroaryl (e.g., a ring containing 1-4 heteroatoms comprising O, N, or S).

In any of the embodiments disclosed herein as applicable, Ring A is phenyl. In any of the embodiments disclosed herein as applicable, Ring A is a 5-6 membered heteroaryl.

In any of the embodiments disclosed herein as applicable, Ring A is a 5-membered heteroaryl group.

In any of the embodiments disclosed herein as applicable, Ring A is a 6-membered heteroaryl group, as described herein.

In any of the embodiments disclosed herein as applicable, Ring A is unsubstituted.

In any of the embodiments disclosed herein as applicable, Ring A is substituted, as described herein.

In any of the embodiments disclosed herein as applicable, Ring A is substituted with at least 1 R₃ (e.g., 2, 3, or 4 R₃), as described herein.

In any of the embodiments disclosed herein as applicable, L is —C(R_{L1}R_{L2})—, and R_{L1} and R_{L2} are each independently selected from H and D.

In any of the embodiments disclosed herein as applicable, L is —C(R_{L1}R_{L2})—, and R_{L1} and R_{L2} are H.

In any of the embodiments disclosed herein as applicable, L is —C(R_{L1}R_{L2})—, and at least one of R_{L1} and R_{L2} is C₁₋₃ alkyl.

In any of the embodiments disclosed herein as applicable, L is —CH₂— optionally substituted with 1 or 2 D.

In any of the embodiments disclosed herein as applicable, L is —CH₂—.

In any of the embodiments disclosed herein as applicable, L is —CD₂-.

In any of the embodiments disclosed herein as applicable, L is —CHD—.

In some embodiments, L is selected from —CH₂— and —CD₂-.

In some embodiments, R₁ and R₂ are each independently selected from oxo, H, F, Cl, Br, I, and CN.

In any of the embodiments disclosed herein as applicable, R₁ and R₂ are H.

In some embodiments, R₁ is selected from oxo, H, F, Cl, Br, I, and CN.

In some schemes of the present invention, R₂ is selected from the oxo, F, Cl, and CN.

In some embodiments, R₂ is selected from oxo, H, F, Cl, Br, I, and CN.

In some embodiments, R₁ is selected from oxo, F, Cl, and CN.

In some embodiments, each R₃ is independently selected from F, Cl, Br, I, OH, NH₂, CN, C₁₋₃ alkyl (e.g., methyl (C₁), ethyl (C₂), n-propyl (C₃) or isopropyl (C₃)), C₁₋₃ alkoxy (e.g., methoxy (C₁), ethoxy (C₂), propoxy (C₃) or isopropyloxy (C₃)), C₁₋₃-alkylamino (e.g., ethylamino, ethylamino, n-propylamino or isopropylamino), diC₁₋₃-alkylamino (e.g., dimethylamino, diethylamino, din-propyl) Amino, diisopropylamino, methyl ethylamine, methyl n-propylamino, methyl isopropylamino, ethyl n-propyl amino, ethyl isopropylamino or n-propyl isopropylamino), C₂₋₄ alkenyl (e.g., vinyl (C₂), 1-allyl (C₃), 2-allyl (C₃), 1-butenyl (C₄), 2-butenyl (C₄) or butadienyl (C₄)), C₂₋₄ alkynyl (e.g. acetenyl (C₂), 1-propinyl (C₃), 2-propinyl (C₃), 1-butylenyl (C₄) or 2-butylenyl (C₄)) and C₃₋₅ cycloalkyl (e.g., cyclopropyl (C₃), cyclobutyl (C₄), cyclopentyl (C₅)), wherein the C₁₋₃alkyl, C₁₋₃alkoxy, C₁₋₃alkylamino, diC₁₋₃alkylamino, C₂₋₄alkyl, C₂₋₄ alkyl and C₃₋₅ alkoxy are each independently optionally substituted with 1, 2, 3, 4, or 5 Rₐ.

In some embodiments, each R₃ is independently selected from F, OH, NH₂, CF₃, OCH₃, —C≡CCH₃, —C≡CCH₂F, —C≡CCHF₂, —C≡CCF₃, —C≡CCD₃, —C≡CH, —C≡CCl, —C≡CF and cyclopropyl.

In some embodiments, each Rₐ is independently selected from D, F, Cl, Br, and I.

In some embodiments, each Rₐ is independently selected from D, F, and I.

In some embodiments, m is selected from 0, 1, 2, 3, 4, and 5.

In any of the embodiments disclosed herein as applicable, m is 0.

In any of the embodiments disclosed herein as applicable, m is 1.

In any of the embodiments disclosed herein as applicable, m is 2.

In any of the embodiments disclosed herein as applicable, m is 3.

In any of the embodiments disclosed herein as applicable, m is 4.

In any of the embodiments disclosed herein as applicable, m is 5.

In some embodiments, R₄, R₅, R₆, R₇, R_{6′}, and R_{7′} are each independently selected from oxo, H, F, Cl, Br, I, OH, NH₂, CN, and C₁₋₃ alkyl (such as methyl (C₁), ethyl (C₂), n-propyl (C₃), or isopropyl (C₃)), C₂₋₄ alkenyl (such as vinyl (C₂), 1-propenyl (C₃), 2-propenyl (C₃), 1-butenyl (C₄), 2-butenyl (C₄), or butadienyl (C₄)), C₁₋₃ alkoxy (such as methoxy (C₁), ethoxy (C₂), propoxy (C₃), or isopropoxy (C₃)), —C(═O)—Rd, —C(═O)—NR_{b1}R_{b2}, and =NO(C₁₋₃ alkyl), wherein the C₁₋₃ alkyl, C₂₋₄ alkenyl, and Cia alkoxy are each independently optionally substituted by 1, 2, 3, 4, or 5 R_{b}.

In any of the embodiments disclosed herein as applicable, R₄, R₅, R₆, R₇, R_{6′} and R_{7′} independently are H.

In any of the embodiments disclosed herein as applicable, at least one of R₄, R₅, R₆, R₇, R_{6′} and R_{7′} is not H.

In any of the embodiments disclosed herein as applicable, R₆ and R₇ together with the carbon atoms to which they are attached form a 3-5 membered heterocyclyl comprising 1-2 heteroatoms selected from N, O and S.

In any of the embodiments disclosed herein as applicable, R₆ and R₇ together with the carbon atoms to which they are attached form a 3-membered heterocyclyl, comprising 1 heteroatom selected from N, O and S.

In any of the embodiments disclosed herein as applicable, R₆ and R₇ together with the carbon atoms to which they are attached form a 3-membered heterocyclyl, wherein the heterocyclyl comprises 1 heteroatom selected from N and O.

In any of the embodiments disclosed herein as applicable, R₆ and R₇ together with the carbon atoms to which they are attached form a 4-membered heterocyclyl, wherein the heterocyclyl comprises 1 heteroatom selected from N, O and S.

In any of the embodiments disclosed herein as applicable R₆ and R₇ together with the carbon atoms to which they are attached form a 4-membered heterocyclyl, wherein the heterocyclyl comprises 1-2 heteroatoms selected from N and O. In any of the embodiments disclosed herein as applicable, R₆ and R₇ together with the carbon atoms to which they are attached form a 4-membered heterocyclyl, wherein the heterocyclyl comprises 1 heteroatom selected from N and O.

In any of the embodiments disclosed herein as applicable, R₆ and R₇ together with the carbon atoms to which they are attached form a 5-membered heterocyclyl, wherein the heterocyclyl comprises 1-2 heteroatoms selected from N, O and S.

In any of the embodiments disclosed herein as applicable, R₆ and R₇ together with the carbon atoms to which they are attached form a 5-membered heterocyclyl, wherein the heterocyclyl comprises 1-2 heteroatoms selected from N and O.

In any of the embodiments disclosed herein as applicable, R₆ and R₇ together with the carbon atoms to which they are attached form a 5-membered heterocyclyl, wherein the heterocyclyl comprises 1-2 heteroatoms selected from N and O.

In some embodiments, each R_{b} is independently selected from D, F, Cl, Br, I, OH, NH₂, CN, C₁₋₃ alkoxy (e.g., methoxy (C₁), ethoxy (C₂), propoxy (C₃) or isopropoxy (C₃)), and —C(═O))—NR_{b1}R_{b2}.

In some embodiments, each R_{b} is independently selected from D, F, Cl, OH, NH₂, CN, C₁₋₃ alkoxy (e.g., methoxy (C₁), ethoxy (C₂), propoxy (C₃) or isopropoxy (C₃)), and —C(═O)—NR_{b1}R_{b2}.

In some embodiments, R₈ is selected from H, F, Cl, Br, I, OH, NH₂, CN, C₁₋₃ alkyl (such as methyl (C₁), ethyl (C₂), n-propyl (C₃), or isopropyl (C₃)), and C₁₋₃ alkoxy (such as methoxy (C₁), ethoxy (C₂), propoxy (C₃), or isopropoxy (C₃)), wherein the C₁₋₃ alkyl and C₁₋₃ alkoxy groups independently are optionally substituted with 1, 2, 3, 4, or 5 Rₐ.

In any of the embodiments disclosed herein as applicable, R₈ is H.

In any of the embodiments disclosed herein as applicable, R₈ is selected from F, Cl, Br, I, OH, NH₂, CN, C₁₋₃ alkyl (such as methyl (C₁), ethyl (C₂), n-propyl (C₃), or isopropyl (C₃)), and C₁₋₃ alkoxy (such as methoxy (C₁), ethoxy (C₂), propoxy (C₃), or isopropoxy (C₃)), wherein the C₁₋₃ alkyl and C₁₋₃ alkoxy are each independently optionally substituted with 1, 2, 3, 4, or 5a.

In any of the embodiments disclosed herein as applicable, Rx is selected from F, Cl, Br, I, OH, NH₂, N, C₁₋₃ alkyl (such as methyl (C₁), ethyl (C₂), n-propyl (C₃), or isopropyl (C₃), and C₁₋₃ alkoxy (such as methoxy (C₁), ethoxy (C₂), propoxy (C₃), or isopropoxy (C₃)), wherein the C₁₋₃ alkyl and C₁₋₃ alkoxy are each independently optionally substituted with 1, 2, 3, 4, or 5a.

In any of the embodiments disclosed herein as applicable, R₈ and R_{8′} together with the carbon atoms to which they are attached form a C₃₋₅ cycloalkyl (e.g., cyclopropyl (C₃), cyclobutyl (C₄), cyclopentyl (C₅)) or 3-5 membered heterocyclyl (e.g., heterocyclyl includes a ring comprising 1-3 heteroatoms selected from O, N and S), wherein the C₃₋₅ cycloalkyl or 3-5 membered heterocyclyl is independently optionally substituted with 1, 2 or 3 R₁₀.

In any of the embodiments disclosed herein as applicable, R₈ and R_{8′} together with the carbon atoms to which they are attached form a C₃₋₅ cycloalkyl group (e.g., cyclopropyl (C₃), cyclobutyl (C₄), cyclopentyl (C₅) wherein the cycloalkyl group is optionally substituted with 1, 2, or 3 R₁₀.

In any of the embodiments disclosed herein as applicable, R₈ and R_{8′} together with the carbon atoms to which they are attached form a 3-5 membered heterocyclyl (e.g., a 3-5 heterocyclyl includes a ring comprising 1 to 2 heteroatoms selected from O, N, and S), wherein the 3-5 membered heterocyclyl is independently optionally substituted with 1, 2, or 3 R₁₀.

In any of the embodiments disclosed herein as applicable, R₈ and R_{8′} together with the carbon atoms to which they are attached form a 3-membered heterocyclyl, wherein the heterocyclyl comprises 1 heteroatom selected from N, O and S.

In any of the embodiments disclosed herein as applicable, R₈ and R_{8′} together with the carbon atoms to which they are attached form a 3-membered heterocyclyl comprising 1 heteroatom selected from N and O.

In any of the embodiments disclosed herein as applicable, R₈ and R_{8′} together with the carbon atoms to which they are attached form a 4-membered heterocyclyl comprising 1-2 heteroatoms selected from N, O and S.

In any of the embodiments disclosed herein as applicable, R₈ and R_{8′} together with the carbon atoms to which they are attached form a 4-membered heterocyclyl group comprising 1-2 heteroatoms selected from N and O.

In any of the embodiments disclosed herein as applicable, R₈ and R_{8′} together with the carbon atoms to which they are attached form a 4-membered heterocyclyl comprising one heteroatom selected from N and O.

In any of the embodiments disclosed herein as applicable, R₈ and R_{8′} together with the carbon atoms to which they are attached form a 5-heterocyclyl comprising 1-2 heteroatoms selected from N, O and S.

In any of the embodiments disclosed herein as applicable, R₈ and R_{8′} together with the carbon atoms to which they are attached form a 5-heterocyclyl group comprising 1-2 heteroatoms selected from N and O.

In any of the embodiments disclosed herein as applicable, R₈ and R_{8′} together with the carbon atoms to which they are attached form a 5-membered heterocyclyl comprising one heteroatom selected from N and O.

In any of the embodiments disclosed herein as applicable, R₉ is selected from —C(═O)—NR_{b3}R_{b4} and —CH₂R_{c}.

In any of the embodiments disclosed herein as R₉ is —C(═O)—NR_{b3}R_{b4}.

In any of the embodiments disclosed herein as applicable, R₉ is —CH₂R_{c}.

In any of the embodiments disclosed herein as applicable, R₉ is —C(═O)—NR_{b1}R_{b2}.

In some embodiments, each R₁₀ is independently selected from oxo, D, F, Cl, Br, I, OH, NH₂, CN, C₁₋₃ alkyl (e.g., methyl (C₁), ethyl (C₂), n-propyl (C₃) or isopropyl (C₃)), C₁₋₃ alkoxy (e.g., methoxy (C₁), ethoxy (C₂), propoxy (C₃) or isopropyloxy (C₃)), C₁₋₃ alkylamino (e.g., methylamino, ethylamino, n-propylamino, or isopropylamino)), diC₁₋₃ alkylamino (e.g., dimethylamino, diethylamino, di-n-propylamino, diisopropylamino, methyl ethylamine, methyl n-propylamino, methyl isopropylamino, ethyl n-propyl amino, ethyl isopropylamino or n-propyl isopropylamino), —C(═O)—Rₐ, —S—R_{d}, —S(═O)—R_{d}, —S(═O)₂—R_{d}, —NH—C(═O)—R_{d}, C₆₋₁₀ aryl, and 5-10 membered heteroaryl, wherein the Cia alkyl is optionally substituted with 1, 2, or 3 OH or F, and the C₆₋₁₀ aryl and 5-10 membered heteroaryl are each independently optionally substituted with 1, 2, 3, 4, or 5 Rₛ₁.

In some embodiments, at least one R₁₀ is independently selected from oxo, D, F, Cl, Br, I, OH, NH₂, CN, C₁₋₃ alkyl (e.g., methyl (C₁), ethyl (C₂), n-propyl (C₃) or isopropyl (C₃)), C₁₋₃ alkoxy (e.g., methoxy (C₁), ethoxy (C₂), propoxy (C₃) or isopropyloxy (C₃)), C₁₋₃ alkylamino (e.g., methylamino, ethylamino, n-propylamino, or isopropylamino)), diC₁₋₃ alkylamino (e.g., dimethylamino, diethylamino, di-n-propylamino, diisopropylamino, methyl ethylamine, methyl n-propylamino, methyl isopropylamino, ethyl n-propyl amino, ethyl isopropylamino or n-propyl isopropylamino), —S—R_{d}, —S(═O)—R_{d}, —S(═O)₂—R_{d} and —NH—C(═O)—R_{d}, wherein the C₁₋₃ alkyl is optionally substituted with 1, 2, or 3 OH or F.

In some embodiments, at least one R₁₀ is independently selected from oxo, D, F. CI, OH, NH₂, CN, C₁₋₃ alkyl (e.g., methyl (C₁), ethyl (C₂), n-propyl (C₃) or isopropyl (C₃)), C₁₋₃ alkoxy (e.g., methoxy (C₁), ethoxy (C₂), propoxy (C₃) or isopropyloxy (C₃)), C₁₋₃ alkylamino (e.g., methylamino, ethylamino, n-propylamino, or isopropylamino)), diC₁₋₃ alkylamino (e.g., dimethylamino, diethylamino, di-n-propylamino, diisopropylamino, methyl ethylamine, methyl n-propylamino, methyl isopropylamino, ethyl n-propyl amino, ethyl isopropylamino or n-propyl isopropylamino), —S—R_{d}, —S(═O)—R_{d}, —S(═O)₂—R_{d} and —NH—C(═O)—R_{d}, wherein the C₁₋₃ alkyl is optionally substituted with 1, 2, or 3 OH or F.

In some embodiments, at least one R₁₀ is independently selected from oxo, F, Cl, C₁₋₃ alkyl (e.g., methyl (C₁), ethyl (C₂), n-propyl (C₃) or isopropyl (C₃)), C₁₋₃ alkoxy (e.g., methoxy (C₁), ethoxy (C₂), propoxy (C₃) or isopropyloxy (C₃)), C₁₋₃ alkylamino (e.g., methylamino, ethylamino, n-propylamino, or isopropylamino)), diC₁₋₃ alkylamino (e.g., dimethylamino, diethylamino, di-n-propylamino, diisopropylamino, methyl ethylamine, methyl n-propylamino, methyl isopropylamino, ethyl n-propyl amino, ethyl isopropylamino or n-propyl isopropylamino), wherein the C₁₋₃ alkyl is optionally substituted with 1, 2, or 3 OH or F.

In some embodiments, at least one R₁₀ is selected from —C(═O)—R_{d}, —S—R_{d}, —S(═O)—R_{d}, —S(═O)₂—R_{d} and —NH—C(═O)—R_{d}.

In some schemes of the present invention, at least one R₁₀ is selected from C₆₋₁₀ aryl and 5-10 membered heteroaryl, wherein the C₆₋₁₀ aryl and 5-10 membered heteroaryl are each independently optionally substituted with 1, 2, 3, 4, or 5 Rₛ₁.

In any of the embodiments disclosed herein as applicable, R_{b1} and R_{b2} have at least one group selected from C₁₋₆ alkyl (e.g., methyl (C₁), ethyl (C₂), n-propyl (C₃), isopropyl (C₃), n-butyl (C₄), isobutyl (C₄), m-butyl (C₄), tert-butyl (C₄), pentyl (C₅) or hexyl (C₆)), C₁₋₆ alkoxy (e.g., methoxy (C₁), ethoxy (C₂), propoxy (C₅), i-propoxy (C₃), butoxy (C₄), i-butoxy (C₄), s-butoxy (C₄), t-butoxy (C₄), pentoxy (C₅), hexyloxy (C₆)), C₃₋₁₀cycloalkyl (e.g., cyclopropyl (C₃), cyclobutyl (C₄), cyclopentyl (C₅), cyclohexyl (C₆), cycloheptyl (C₇), cyclooctyl (C₈), bicyclic [2.2.1]heptyl (C₇), bicyclic [2.2.2] octyl (C₈), cyclononyl (C₉), cyclodecyl (C₁₀), octahydro-1H-indenyl (C₉), decahydronaphthyl (C₁₀) or spiro[4.5]decyl (C₁₀)), 3-10 heterocyclyl (e.g., heterocyclyl includes one or two 3-8 membered rings comprising 1-5 heteroatoms selected from N, O and S), C₆₋₁₀ aryl (e.g., phenyl or naphthyl) and 5-10 membered heteroaryl (e.g., heteroaryl comprises one or two 5-membered or 6-membered rings comprising 1-5 heteroatoms selected from N, O and S), wherein the C₁₋₆ alkyl, C₁₋₆ alkoxy, C₃₋₁₀cycloalkyl, 3-10 membered heterocyclyl, C₆₋₁₀ aryl and 5-10 membered heteroaryl are each independently optionally substituted with 1, 2, 3 or 4 Rₑ₁.

In any of the embodiments disclosed herein as applicable, R_{b1} and R_{b2} have at least one group selected from C₁₋₆ alkyl (e.g., methyl (C₁), Ethyl (C₂), N-propyl (C₃), Isopropyl (C₃), n-butyl (C₄), Isobutyl (C₄), m-butyl (C₄), tert-butyl (C₄), pentyl (C₅) or hexyl (C₆)), C₁₋₆ alkoxy (e.g., methoxy (C₁), ethoxy (C₂), propoxy (C₃), i-propoxy (C₃), butoxy (C₄), i-butoxy (C₄), s-butoxy (C₄), t-butoxy (C₄), pentoxy (C₅), hexyloxy (C₆)), C₃₋₁₀cycloalkyl (e g., cyclopropyl (C₃), cyclobutyl (C₄), cyclopentyl (C₅), cyclohexyl (C₆), ring heptyl (C₇), cyclooctyl (C₈), Double ring [2.2.1]Heptyl (C₇), Double ring[2.2.2]octyl (C₈), Cyclinyl (C₉), ring decyl (C₁₀), octahydro-1H-indenyl (C₉), decahydronaphthyl (C₁₀) or spiro[4.5]decyl (C₁₀)), 3-10 membered heterocyclyl (e.g., heterocyclyl includes one or two 3-8 membered rings comprising 1-5 heteroatoms selected from N, O and S), wherein the heterocyclyl may be monocyclic or polycyclic, such as spiro rings, bridging rings or fused polycyclic rings), C₆₋₁₀ aryl (e.g., phenyl or naphthyl) and 5-10 membered heteroaryl (e.g., heteroaryl comprises one or two 5-membered or 6-membered rings comprising 1-5 heteroatoms selected from N, O and S), wherein the C₁₋₆ alkyl, C₁₋₆ alkoxy, C₃₋₁₀cycloalkyl, 3-10 membered heterocyclyl, C₆₋₁₀ aryl and 5-10 membered heteroaryl are each independently optionally substituted with 1, 2, 3 or 4 Rₑ₁.

In any of the embodiments disclosed herein as applicable, R_{b1} and R_{b2} have at least one group selected from C₁₋₆ alkyl (e.g., methyl (C₁), ethyl (C₂), n-propyl (C₃), isopropyl (C₃), n-butyl (C₄), isobutyl (C₄), m-butyl (C₄), tert-butyl (C₄), pentyl (C₅) or hexyl (C₆)), and C₁₋₆ alkoxy (e.g., methoxy (C₁), ethoxy (C₂), propoxy (C₃), i-propoxy (C₃), butoxy (C₄), i-butoxy (C₄), s-butoxy (C₄), t-butoxy (C₄), pentoxy (C₅), hexyloxy (C₆)), wherein the C₁₋₆ alkyl and C₁₋₆ alkoxy are each independently optionally substituted with 1, 2, 3 or 4 Rₑ₁.

In any of the embodiments disclosed herein as applicable, R_{b1} and R_{b2} have at least one group selected from C₃₋₁₀cycloalkyl (e.g., cyclopropyl (C₃), cyclobutyl (C₄), cyclopentyl (C₅), cyclohexyl (C₆), cycloheptyl (C₇), cyclooctyl (C₈), bicyclic [2.2.1]heptyl (C₇), bicyclic [2.2.2]octyl (C₈), cyclononyl (C₉), cyclodecyl (C₁₀), octahydro-1H-indenyl (C₉), decahydronaphthyl (C₁₀) or spiro[4.5]decyl (C₁₀)), 3-10 heterocyclyl (e.g., heterocyclyl includes one or two 3-8 membered rings comprising 1-5 heteroatoms selected from N, O and S), C₆₋₁₀ aryl (e.g., phenyl or naphthyl) and 5-10 membered heteroaryl (e.g., heteroaryl comprises one or two 5-membered or 6-membered rings comprising 1-5 heteroatoms selected from N, O and S), wherein the C₃₋₁₀cycloalkyl, 3-10 membered heterocyclyl, C₆₋₁₀ aryl and 5-10 membered heteroaryl are each independently optionally substituted with 1, 2, 3 or 4 Rₑ₁.

In any of the embodiments disclosed herein as applicable, R_{b1} and R_{b2} have at least one group selected from C₃₋₆cycloalkyl (e.g., cyclopropyl (C₃), cyclobutyl (C₄), cyclopentyl (C₅), Cyclohexyl (C₆)), 3-6 heterocyclyl, C₆ aryl and 5-6 membered heteroaryl (e.g., heteroaryl comprises 1-3 heteroatoms selected from N, O and S), wherein the C₃₋₆ cycloalkyl, 3-6 membered heterocyclyl, C₆ aryl and 5-6 membered heteroaryl are each independently optionally substituted with 1, 2, 3 or 4 Rₑ₁.

In any of the embodiments disclosed herein as applicable, R_{b1} and R_{b2} together with the nitrogen atoms to which they are attached form a 3-6 membered heterocyclyl (e.g., heterocyclyl comprises 1-4 heteroatoms selected from N, O and S), wherein the 3-6 membered heterocyclyl is optionally substituted with 1, 2, 3, or 4 Rₑ₁.

In any of the embodiments disclosed herein as applicable, R_{b3} and R_{b4} are each independently H, C₁₋₆ alkyl, C₁₋₆ alkoxy (e.g., methoxy (C₁), ethoxy (C₂), propoxy (C₃), i-propoxy (C₃), butoxy (C₄), i-butoxy (C₄), s-butoxy (C₄), t-butoxy (C₄), pentoxy (C₅), hexyloxy (C₆)), C₃₋₁₀ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 10-membered heterocyclyl (e.g., heterocyclyl comprising one or two 3- to 8-membered rings comprising 1-5 heteroatoms selected from N, O, and S), C₆₋₁₀ aryl (e.g., phenyl or naphthyl), 5- to 10-membered heteroaryl (e.g., heteroaryl comprising one or two 5- or 6-membered rings and 1-5 heteroatoms selected from N, O, and S), wherein the alkyl, alkoxy, carbocyclyl, heterocyclyl, aryl, or heteroaryl is optionally substituted with 1, 2, 3, or 4 Rₑ₂.

In any of the embodiments disclosed herein as applicable, at least one of R_{b1} and R_{b2} is C₁₋₆ alkyl or C₁₋₆ alkoxy (e.g., methoxy (C₁), ethoxy (C₂), propoxy (C₃), i-propoxy (C₃), butoxy (C₄), i-butoxy (C₄), s-butoxy (C₄), t-butoxy (C₄), pentoxy (C₅), hexyloxy (C₆)), wherein the alkyl or alkoxy is optionally substituted with 1, 2, 3, or 4 Rₑ₂.

In any of the embodiments disclosed herein as applicable, at least one of R_{b1} and R_{b2} is C₃₋₁₀ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 10-membered heterocyclyl (e.g., heterocyclyl comprising one or two 3- to 8-membered rings and 1-5 heteroatoms selected from N, O, and S, wherein the heterocyclyl can be monocyclic or polycyclic such as spiro, bridged, or fused polycyclic ring), C₆₋₁₀ aryl (e.g., phenyl or naphthyl), 5- to 10-membered heteroaryl (e.g., heteroaryl comprising one or two 5- or 6-membered rings and 1-5 heteroatoms selected from N, O, and S), wherein the carbocyclyl, heterocyclyl, aryl, or heteroaryl is optionally substituted with 1, 2, 3, or 4 Rₑ₂.

In any of the embodiments disclosed herein as applicable, at least one of R_{b1} and R_{b2} is C₃₋₆ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆)), 3- to 6-membered heterocyclyl (e.g., heterocyclyl comprising 1-3 heteroatoms selected from N, O, and S), C₆ aryl, 5- to 6-membered heteroaryl (e.g., heteroaryl comprising 1-4 heteroatoms selected from N, O, and S), wherein the carbocyclyl, heterocyclyl, aryl, or heteroaryl is optionally substituted with 1, 2, 3, or 4 Rₑ₂.

In any of the embodiments disclosed herein as applicable, R_{b3} and R_{b4}, together with nitrogen atom to which they are attached, form 3-6 membered heterocyclyl (e.g., heterocyclyl comprising 1-4 heteroatoms selected from N, O, and S), wherein the 3-6 membered heterocyclyl is optionally substituted with 1, 2, 3, or 4 Rₑ₂.

In any of the embodiments disclosed herein as applicable, R is selected from F, Cl, Br, I, OH, NH₂, —(C═O)NR_{C1}R_{C2}, —O(C═O)NR_{C1}R_{C2}, —NR_{C0}(C═O)Rₑ₁, and —NR_{C0}(C═O)NR_{C1}R_{C2}. In some embodiments, R_{c} is selected from the group consisting of F, Cl, OH, NH₂, —O(C═O)NR_{CI}R_{C2}, —NR_{C0}(C═O)R_{C1}, and —NR_{C0}(C═O)NR_{C1}R_{C2}. In some embodiments, R_{c} is selected from F, Cl, —O(C═O)NR_{C1}R_{C2}, —NR_{C0}(C═O)R_{C1}, and —NR_{C0}(C═O)NR_{C1}R_{C2}. In some embodiments, R_{c} is selected from the group consisting of F, Cl, OH, and NH₂. In some embodiments, R_{c} is selected from —O(C═O)NR_{C1}R_{C2}, —NR_{C0}(C═O)R_{C1}, and —NR_{C0}(C═O)NR_{C1}R_{C2}.

In any of the embodiments disclosed herein as applicable, R_{C0}, R_{C1} and R_{C2} are each independently selected from H, C₁₋₆ alkyl (e.g., methyl (C₁), ethyl (C₂), n-propyl (C₃), isopropyl (C₃), n-butyl (C₄), isobutyl (C₄), metabutyl (C₄), tert-butyl (C₄), pentyl (C₅) or hexyl (C₆)), C₃₋₆ cycloalkyl (e.g., cyclopropyl (C₃), cyclobutyl (C₄), cyclopentyl (C₅), cyclohexyl (C₆)) and 3-6 membered heterocyclyl (e.g., heterocyclyl includes one or two 3-6 membered rings comprising 1-4 heteroatoms selected from N, O and S).

In any of the embodiments disclosed herein as applicable, R_{d} is selected from C₁₋₃ alkyl (e.g., methyl (C₁), ethyl (C₂), n-propyl (C₃) or isopropyl (C₃)).

In any of the embodiments disclosed herein as applicable, Rₑ₁ is F, Cl, Br, I, OH, NH₂, NO₂, C₁₋₃ alkyl (e.g., methyl (C₁), ethyl (C₂), n-propyl (C₃), or i-propyl (C₃)), C₁₋₃ alkylamino (e.g., methylamino, ethylamino, n-propylamino, or i-propylamino), di-C₁₋₃ alkylamino (e.g., dimethylamino, diethylamino, di-n-propylamino, di-i-propylamino, methylethylamino, methyl-n-propylamino, methyl-i-propylamino, ethyl-n-propylamino, ethyl-i-propylamino, or n-propyl-i-propylamino), CN, C₁₋₃ alkoxy (e.g., methoxy (C₁), ethoxy (C₂), propoxy (C₃), or i-propoxy (C₃)), —S(═O)₂—(C₁₋₃ alkyl), —(C═O)(C₁₋₃ alkyl), —(C=O)O(C₁₋₃ alkyl), —(C═O)NH(C₁₋₃ alkyl), —(C═O)N(C₁₋₃ alkyl)₂, C₃₋₁₀ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 10-membered heterocyclyl (e.g., heterocyclyl comprising one or two 3- to 8-membered rings and 1-5 heteroatoms selected from N, O, and S, wherein the heterocyclyl can be monocyclic or polycyclic such as spiro, bridged, or fused polycyclic ring), C₆₋₁₀ aryl (e.g., phenyl or naphthyl), 5- to 10-membered heteroaryl (e.g., heteroaryl comprising one or two 5- or 6-membered rings and 1-5 heteroatoms selected from N, O, and S).

In any of the embodiments disclosed herein as applicable, Rₑ₁ is F, Cl, Br, I, OH, NH₂, NO₂, C₁₋₃ alkyl (e.g., methyl (C₁), ethyl (C₂), n-propyl (C₃), or i-propyl (C₃)), C₁₋₃ alkylamino (e.g., methylamino, ethylamino, n-propylamino, or i-propylamino), di-C₁₋₃ alkylamino (e.g., dimethylamino, diethylamino, di-n-propylamino, di-i-propylamino, methylethylamino, methyl-n-propylamino, methyl-i-propylamino, ethyl-n-propylamino, ethyl-i-propylamino, or n-propyl-i-propylamino), CN, C₁₋₃ alkoxy (e.g., methoxy (C₁), ethoxy (C₂), propoxy (C₃), or i-propoxy (C₃)), —S(═O)₂—(C₁₋₃ alkyl), —(C═O)(C₁₋₃ alkyl), —(C=O)O(C₁₋₃ alkyl), —(C═O)NH(C₁₋₃ alkyl), or —(C═O)N(C₁₋₃ alkyl)₂.

In any of the embodiments disclosed herein as applicable, Ret is F, Cl, Br, I, OH, NH₂, NO₂, C₁₋₃ alkyl (e.g., methyl (C₁), ethyl (C₂), n-propyl (C₃), or i-propyl (C₃)), C₁₋₃ alkylamino (e.g., methylamino, ethylamino, n-propylamino, or i-propylamino), di-C₁₋₃ alkylamino (e.g., dimethylamino, diethylamino, di-n-propylamino, di-i-propylamino, methylethylamino, methyl-n-propylamino, methyl-i-propylamino, ethyl-n-propylamino, ethyl-i-propylamino, or n-propyl-i-propylamino), CN, or C₁₋₃ alkoxy (e.g., methoxy (C₁), ethoxy (C₂), propoxy (C₃), or i-propoxy (C₃)). In certain embodiments, Rₑ₁ is —S(═O)₂—(C₁₋₃ alkyl), —(C═O)(C₁₋₃ alkyl), —(C=O)O(C₁₋₃ alkyl), —(C═O)NH(C₁₋₃ alkyl), or —(C═O)N(C₁₋₃ alkyl)₂.

In any of the embodiments disclosed herein as applicable, Rₑ₁ is selected from F, Cl, Br, I, OH, NH₂, NO₂, C₁₋₃ alkyl (e.g., methyl (C₁), ethyl (C₂), n-propyl (C₃), or i-propyl (C₃)), C₁₋₃ alkylamino (e.g., methylamino, ethylamino, n-propylamino or isopropylamino), diC₁₋₃ alkylamino (e.g., dimethylamino, diethylamino, di-n-propylamino, diisopropylamino, methyl ethylamine, methyl n-propylamino, methyl isopropylamino, ethyl n-propyl amino, ethyl isopropylamino or n-propyl isopropylamino), CN, C₁₋₃ alkoxy (e.g., methoxy (C₁), ethoxy (C₂), propoxy (C₃) or isopropyloxy (C₃), C₃₋₁₀ cycloalkyl (e.g., cyclopropyl (C₃), cyclobutyl (C₄), cyclopentyl (C₅), cyclohexyl (C₆), cycloheptyl (C₇), cyclooctyl (C₈), bicyclic [2.2.1]heptyl (C₇), bicyclic [2.2.2]octyl (C₈), cyclononyl (C₉), cyclodecyl (C₁₀), octahydro-1H-indenyl (C₉), decahydronaphthyl (C₁₀), or spiro[4.5]decyl (C₁₀)), 3-10 membered heterocyclyl, (e.g., heterocyclyl includes one or two 3-8 membered rings comprising 1-5 heteroatoms selected from N, O and S, wherein heterocyclyl may be monocyclic or polycyclic, such as spiro rings, bridging rings or fused polycyclic rings), C₆₋₁₀ aryl (e.g., phenyl or naphthyl) and 5-10 membered heteroaryl (e g., heteroaryl comprises one or two 5-membered or 6-membered rings and 1-5 rings selected from heteroatoms N, O and S).

In any of the embodiments disclosed herein as applicable, Rₑ₁ is C₃₋₁₀ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 10-membered heterocyclyl (e.g., heterocyclyl comprising one or two 3- to 8-membered rings and 1-5 heteroatoms selected from N, O, and S, wherein the heterocyclyl can be monocyclic or polycyclic such as spiro, bridged, or fused polycyclic ring), C₆₋₁₀ aryl (e.g., phenyl or naphthyl), 5- to 10-membered heteroaryl (e.g., heteroaryl comprising one or two 5- or 6-membered rings and 1-5 heteroatoms selected from N, O, and S).

In any of the embodiments disclosed herein as applicable, Rₑ₂ is F, Cl, Br, I, OH, NH₂, NO₂, C₁₋₃ alkyl (e.g., methyl (C₁), ethyl (C₂), n-propyl (C₃), or i-propyl (C₃)), C₁₋₃ alkylamino (e.g., methylamino, ethylamino, n-propylamino, or i-propylamino), di-C₁₋₃ alkylamino (e.g., dimethylamino, diethylamino, di-n-propylamino, di-i-propylamino, methylethylamino, methyl-n-propylamino, methyl-i-propylamino, ethyl-n-propylamino, ethyl-i-propylamino, or n-propyl-i-propylamino), CN, C₁₋₃ alkoxy (e.g., methoxy (C₁), ethoxy (C₂), propoxy (C₃), or i-propoxy (C₃)), —S(═O)₂—(C₁₋₃ alkyl), (C═O)(C₁₋₃ alkyl), —(C=O)O(C₁₋₃ alkyl), —(C═O)NH(C₁₋₃ alkyl), —(C═O)N(C₁₋₃ alkyl)₂, C₃₋₁₀ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 10-membered heterocyclyl (e.g., heterocyclyl comprising one or two 3- to 8-membered rings and 1-5 heteroatoms selected from N, O, and S, wherein the heterocyclyl can be monocyclic or polycyclic such as spiro, bridged, or fused polycyclic ring), C₆₋₁₀ aryl (e.g., phenyl or naphthyl), 5- to 10-membered heteroaryl (e.g., heteroaryl comprising one or two 5- or 6-membered rings and 1-5 heteroatoms selected from N, O, and S), wherein the cycloalkyl, heterocyclyl, aryl, and heteroaryl are each independently optionally substituted with 1, 2, 3, 4, or 5 Rₛ₁, and wherein the alkyl and alkoxy are each independently optionally substituted with 1, 2, 3, 4, or 5 Rₛ₂.

In any of the embodiments disclosed herein as applicable, Rₑ₂ is F, Cl, Br, I, OH, NH₂, NO₂, C₁₋₃ alkyl (e.g., methyl (C₁), ethyl (C₂), n-propyl (C₃), or i-propyl (C₃)), C₁₋₃ alkylamino (e.g., methylamino, ethylamino, n-propylamino, or i-propylamino), di-C₁₋₃ alkylamino (e.g., dimethylamino, diethylamino, di-n-propylamino, di-i-propylamino, methylethylamino, methyl-n-propylamino, methyl-i-propylamino, ethyl-n-propylamino, ethyl-i-propylamino, or n-propyl-i-propylamino), CN, C₁₋₃ alkoxy (e.g., methoxy (C₁), ethoxy (C₂), propoxy (C₃), or i-propoxy (C₃)), —S(═O)₂—(C₁₋₃ alkyl), —(C═O)(C₁₋₃ alkyl), —(C=O)O(C₁₋₃ alkyl), —(C═O)NH(C₁₋₃ alkyl), or —(C═O)N(C₁₋₃ alkyl)₂, wherein the alkyl and alkoxy are each independently optionally substituted with 1, 2, 3, 4, or 5 Rₛ₂.

In any of the embodiments disclosed herein as applicable, Rex is F, Cl, Br, I, OH, NH₂, NO₂, C₁₋₃ alkyl (e.g., methyl (C₁), ethyl (C₂), n-propyl (C₃), or i-propyl (C₃)), C₁₋₃ alkylamino (e.g., methylamino, ethylamino, n-propylamino, or i-propylamino), di-C₁₋₃ alkylamino (e.g., dimethylamino, diethylamino, di-n-propylamino, di-i-propylamino, methylethylamino, methyl-n-propylamino, methyl-i-propylamino, ethyl-n-propylamino, ethyl-i-propylamino, or n-propyl-i-propylamino), CN, or C₁₋₃ alkoxy (e.g., methoxy (C₁), ethoxy (C₂), propoxy (C₃), or i-propoxy (C₃)), wherein the alkyl and alkoxy are each independently optionally substituted with 1, 2, 3, 4, or 5 Rₛ₂.

In any of the embodiments disclosed herein as applicable, Rₑ₂ is —S(═O)₂—(C₁₋₃ alkyl), —(C═O)(C₁₋₃ alkyl), —(C═O)O(C₁₋₃ alkyl), —(C═O)NH(C₁₋₃ alkyl), or —(C═O)N(C₁₋₃ alkyl)₂.

In any of the embodiments disclosed herein as applicable, Rₑ₂ is F, Cl, Br, I, OH, NH₂, NO₂, C₁₋₃ alkyl (e.g., methyl (C₁), ethyl (C₂), n-propyl (C₃), or i-propyl (C₃)), C₁₋₃ alkylamino (e.g., methylamino, ethylamino, n-propylamino, or i-propylamino), di-C₁₋₃ alkylamino (e.g., dimethylamino, diethylamino, di-n-propylamino, di-i-propylamino, methylethylamino, methyl-n-propylamino, methyl-1-propylamino, ethyl-n-propylamino, ethyl-1-propylamino, or n-propyl-1-propylamino), CN, C₁₋₃ alkoxy (e.g., methoxy (C₁), ethoxy (C₂), propoxy (C₃), or i-propoxy (C₃)), C₃₋₁₀ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 10-membered heterocyclyl (e.g., heterocyclyl comprising one or two 3- to 8-membered rings and 1-5 heteroatoms selected from N, O, and S, wherein the heterocyclyl can be monocyclic or polycyclic such as spiro, bridged, or fused polycyclic ring), C₆₋₁₀ aryl (e.g., phenyl or naphthyl), 5- to 10-membered heteroaryl (e.g., heteroaryl comprising one or two 5- or 6-membered rings and 1-5 heteroatoms selected from N, O, and S), wherein the cycloalkyl, heterocyclyl, aryl, and heteroaryl are each independently optionally substituted with 1, 2, 3, 4, or 5 Rₛ₁, and wherein the alkyl and alkoxy are each independently optionally substituted with 1, 2, 3, 4, or 5 Rₛ₂.

In any of the embodiments disclosed herein as applicable, Rₑ₂ is C₃₋₁₀ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₉), or spiro[4.5]decanyl (C₁₀)), 3- to 10-membered heterocyclyl (e.g., heterocyclyl comprising one or two 3- to 8-membered rings and 1-5 heteroatoms selected from N, O, and S, wherein the heterocyclyl can be monocyclic or polycyclic such as spiro, bridged, or fused polycyclic ring), C₆₋₁₀ aryl (e.g., phenyl or naphthyl), 5- to 10-membered heteroaryl (e.g., heteroaryl comprising one or two 5- or 6-membered rings and 1-5 heteroatoms selected from N, O, and S), wherein the cycloalkyl, heterocyclyl, aryl, and heteroaryl are each independently optionally substituted with 1, 2, 3, 4, or 5 Rₛ₁.

In any of the embodiments disclosed herein as applicable, two Rₑ₂ together with the carbon atoms to which they are attached form a Ce aryl or 5- or 6-membered heteroaryl (e.g., heteroaryl comprising one or two 5- or 6-membered rings and 1-5 heteroatoms selected from N, O, and S).

In any of the embodiments disclosed herein as applicable, Rₛ₁ is oxo, F, Cl, Br, I, OH, NH₂, NO₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁₋₆ alkylamino (e.g., methylamino, ethylamino, n-propylamino, or i-propylamino), di-C₁₋₆ alkylamino (e.g., dimethylamino, diethylamino, di-n-propylamino, di-i-propylamino, methylethylamino, methyl-n-propylamino, methyl-i-propylamino, ethyl-n-propylamino, ethyl-i-propylamino, or n-propyl-i-propylamino), CN, C₁₋₆ alkoxy (e.g., methoxy (C₁), ethoxy (C₂), propoxy (C₃), i-propoxy (C₃), butoxy (C₄), i-butoxy (C₄), s-butoxy (C₄), t-butoxy (C₄), pentoxy (C₅), hexyloxy (C₆)), —S(═O)₂—(C₁₋₃ alkyl), —(C═O)(C₁₋₃ alkyl), —(C=O)O(C₁₋₃ alkyl), —(C═O)NH(C₁₋₃ alkyl), or —(C═O)N(C₁₋₃ alkyl)₂.

In any of the embodiments disclosed herein as applicable, Rₛ₁ is oxo, F, Cl, Br, I, OH, NH₂, NO₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁₋₆ alkylamino (e.g., methylamino, ethylamino, n-propylamino, or i-propylamino), di-C₁₋₆ alkylamino (e.g., dimethylamino, diethylamino, di-n-propylamino, di-i-propylamino, methylethylamino, methyl-n-propylamino, methyl-i-propylamino, ethyl-n-propylamino, ethyl-i-propylamino, or n-propyl-i-propylamino), CN, or C₁₋₆ alkoxy (e.g., methoxy (C₁), ethoxy (C₂), propoxy (C₃), i-propoxy (C₃), butoxy (C₄), i-butoxy (C₄), s-butoxy (C₄), t-butoxy (C₄), pentoxy (C₅), hexyloxy (C₆)).

In any of the embodiments disclosed herein as applicable, Rₛ₁ is C₁₋₆ alkyl (e.g., methyl (C₁), ethyl (C₂), n-propyl (C₂), i-propyl (C₃), n-butyl (C₄), i-butyl (C₄), s-butyl (C₄), t-butyl (C₄), pentyl (C₅), or hexyl (C₆)), C₁₋₆ alkylamino (e.g., methylamino, ethylamino, n-propylamino, or i-propylamino), di-C₁₋₆ alkylamino (e.g., dimethylamino, diethylamino, di-n-propylamino, di-i-propylamino, methylethylamino, methyl-n-propylamino, methyl-i-propylamino, ethyl-n-propylamino, ethyl-i-propylamino, or n-propyl-i-propylamino), CN, C₁₋₆ alkoxy (e.g., methoxy (C₁), ethoxy (C₂), propoxy (C₃), i-propoxy (C₃), butoxy (C₄), i-butoxy (C₄), s-butoxy (C₄), t-butoxy (C₄), pentoxy (C₅), hexyloxy (C₆)), —S(═O)₂—(C₁₋₃ alkyl), —(C═O)(C₁₋₃ alkyl), —(C═O)O(C₁₋₃ alkyl), —(C═O)NH(C₁₋₃ alkyl), or —(C═O)N(C₁₋₃ alkyl)₂.

In any of the embodiments disclosed herein as applicable, Rₛ₁ is oxo, F, Cl, Br, I, OH, NH₂, NO₂, —S(═O)₂—(C₁₋₃ alkyl), —(C═O)(C₁₋₃ alkyl), —(C═O)O(C₁₋₃ alkyl), —(C═O)NH(C₁₋₃ alkyl), or —(C═O)N(C₁₋₃ alkyl)₂.

In any of the embodiments disclosed herein as applicable, Rₛ₁ is —(C=O)(C₁₋₃ alkyl), —(C=O)O(C₁₋₃ alkyl), —(C═O)NH(C₁₋₃ alkyl), or —(C═O)N(C₁₋₃ alkyl)₂.

In any of the embodiments disclosed herein as applicable, Rₛ₂ is F, Cl, Br, I, OH, NH₂, C₁₋₆ alkylamino (e.g., methylamino, ethylamino, n-propylamino, or i-propylamino), di-C₁₋₆ alkylamino (e.g., dimethylamino, diethylamino, di-n-propylamino, di-i-propylamino, methylethylamino, methyl-n-propylamino, methyl-i-propylamino, ethyl-n-propylamino, ethyl-i-propylamino, or n-propyl-i-propylamino), CN, C₁₋₆ alkoxy (e.g., methoxy (C₁), ethoxy (C₂), propoxy (C₃), i-propoxy (C₃), butoxy (C₄), i-butoxy (C₄), s-butoxy (C₄), t-butoxy (C₄), pentoxy (C₅), hexyloxy (C₆)), —S(═O)₂—(C₁₋₃ alkyl), —(C═O)(C₁₋₃ alkyl), —(C=O)O(C₁₋₃ alkyl), —(C═O)NH(C₁₋₃ alkyl), or —(C═O)N(C₁₋₃ alkyl)₂.

In any of the embodiments disclosed herein as applicable, Rₛ₂ is F, Cl, Br, I, OH, NH₂, C₁₋₆ alkylamino (e.g., methylamino, ethylamino, n-propylamino, or i-propylamino), di-C₁₋₆ alkylamino (e.g., dimethylamino, diethylamino, di-n-propylamino, di-i-propylamino, methylethylamino, methyl-n-propylamino, methyl-i-propylamino, ethyl-n-propylamino, ethyl-i-propylamino, or n-propyl-i-propylamino), CN, or C₁₋₆ alkoxy (e.g., methoxy (C₁), ethoxy (C₂), propoxy (C₃), i-propoxy (C₃), butoxy (C₄), i-butoxy (C₄), s-butoxy (C₄), t-butoxy (C₄), pentoxy (C₅), hexyloxy (C₆)).

In any of the embodiments disclosed herein as applicable, Rₛ₂ is C₁₋₆ alkylamino (e.g., methylamino, ethylamino, n-propylamino, or i-propylamino), di-C₁₋₆ alkylamino (e.g., dimethylamino, diethylamino, di-n-propylamino, di-i-propylamino, methylethylamino, methyl-n-propylamino, methyl-i-propylamino, ethyl-n-propylamino, ethyl-i-propylamino, or n-propyl-i-propylamino), CN, C₁₋₆ alkoxy (e.g., methoxy (C₁), ethoxy (C₂), propoxy (C₃), i-propoxy (C₃), butoxy (C₄), i-butoxy (C₄), s-butoxy (C₄), t-butoxy (C₄), pentoxy (C₅), hexyloxy (C₆)), —S(═O)₂—(C₁₋₃ alkyl), —(C═O)(C₁₋₃ alkyl), —(C═O)O(C₁₋₃ alkyl), —(C═O)NH(C₁₋₃ alkyl), or —(C═O)N(C₁₋₃ alkyl)₂.

In any of the embodiments disclosed herein as applicable, Rₛ₂ is F, Cl, Br, I, OH, NH₂, —S(═O)₂—(C₁₋₃ alkyl), —(C═O)(C₁₋₃ alkyl), —(C═O)O(C₁₋₃ alkyl), —(C═O)NH(C₁₋₃ alkyl), or —(C═O)N(C₁₋₃ alkyl)₂.

In any of the embodiments disclosed herein as applicable, Rₛ₂ is —(C═O)(C₁₋₃ alkyl), —(C=O)O(C₁₋₃ alkyl), —(C═O)NH(C₁₋₃ alkyl), or —(C═O)N(C₁₋₃ alkyl)₂.

In certain aspects, the present disclosure provides a compound of Formula (I):

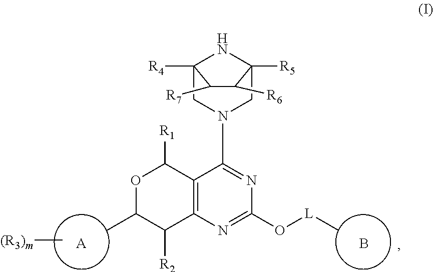

or a stereoisomer thereof, or a pharmaceutically acceptable salt thereof, wherein:
Ring A is selected from C₆₋₁₀ aryl and 5-10 membered heteroaryl;
Ring B is selected from

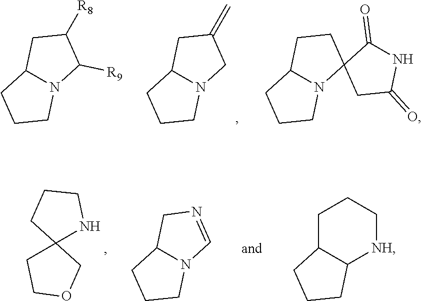

wherein

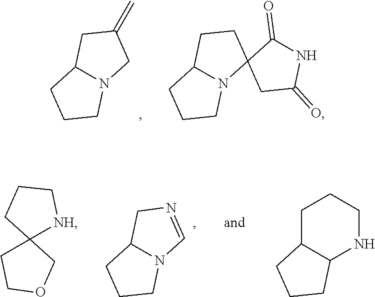

are each independently optionally substituted with 1, 2, 3, or 4 R₁₀;
L is —CH₂—, wherein the —CH₂— is optionally substituted with 1 or 2 D;
R₁ and R₂ are each independently selected from H, F, Cl, Br, I, C₁₋₃ alkyl, and C₃₋₅ cycloalkyl; wherein the C₁₋₃ alkyl and C₃₋₅ cycloalkyl are each independently optionally substituted with 1, 2, or 3 Rₐ;
each R₃ is independently selected from F, Cl, Br, I, OH, NH₂, CN, C₁₋₃ alkyl, C₁₋₃ alkoxy, C₁₋₃ alkylamino, C₂₋₄ alkenyl, C₂₋₄ alkynyl, and C₃₋₅ cycloalkyl; wherein the C₁₋₃alkyl, C₁₋₃alkoxy, C₁₋₃ alkylamino, C₂₋₄alkenyl, C₂₋₄alkynyl and C₃₋₅ cycloalkyl are each independently substituted with 1, 2, 3, 4, or 5 Rₐ;
R₄, R₅, R₆ and R₇ is selected from the group consisting of H, OH, NH₂, CN, C₁₋₃alkyl, and C₁₋₃ alkoxy, wherein the C₁₋₃ alkyl and C₁₋₃ alkoxy are each independently optionally substituted with 1, 2, 3, 4, or 5 Rₐ;
R₈ is selected from the group consisting of H, F, Cl, Br, I, OH, NH₂, CN, Cia alkyl, and C₁₋₃ alkoxy, wherein the C₁₋₃ alkyl and C₁₋₃ alkoxy are each independently optionally substituted with 1, 2, 3, 4, or 5 Rₐ;
R₉ is selected from —C(═O)—NR_{b1}R_{b2} and —CH₂R_{c};
each R₁₀ is independently selected from D, F, Cl, Br, I, OH, NH₂, C₁₋₃ alkyl, C₁₋₃ alkylamino, and —NH—C(═O)—Rₐ;
each Rₐ is independently selected from D, F, Cl, Br, and I;
each R_{b} is independently selected from D, F, Cl, Br, I, OH, NH₂, CN, and —C(═O)—NR_{b1}R_{b2};
R_{b1} and R_{b2} are each independently selected from H and C₁₋₃ alkyl;
R_{c} is selected from F, Cl, Br, I, OH, and NH₂;
R_{d} is C₁₋₃ alkyl;
m is selected from 0, 1, 2, 3, 4 and 5.

In some embodiments, the abovementioned L is selected from the group consisting of —CH₂— and —CD₂-, and other variables are as defined in the present invention.

In some embodiments, Rₐ is independently selected from the group consisting of D, F, and Cl, with other variables as defined in the present invention.

In some embodiments, R_{b} is independently selected from OH, CN, and —C(═O)—NH₂, and other variables are as defined in the present invention.

In some embodiments, R_{b1} and R_{b2} are each independently selected from H and CH₃, and other variables are as defined in the present invention.

In some embodiments, R_{c} is OH.

In some embodiments of the present disclosure, the R_{d} is CH₃, while other variables are as defined herein.

In some embodiments, R₁ is H.

In some embodiments, R₂ is H.

In some embodiments, R₃ is independently selected from F, Cl, Br, I, OH, NH₂, CN, CH₃, CH₂CH₃, OCH₃, OCH₂CH₃, NHCH₃, N(CH₃)₂, —C≡CH, —C≡CCH₃, cyclopropyl and cyclobutyl, wherein the CH₃, CH₂CH₃, OCH₃, OCH₂CH₃, NHCH, N(CH₃)₂, —C≡CH, —C≡CCH₃ cyclopropyl and cyclobutyl are each independently optionally substituted with 1, 2, 3, 4 or 5 Rₐ.

In some embodiments, the present invention provides a compound of formula (I-a)

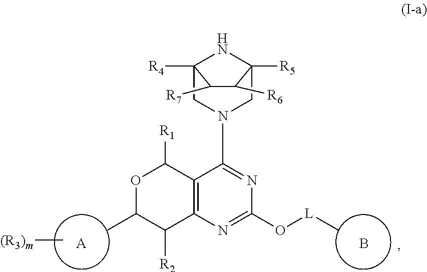

or a pharmaceutically acceptable salt thereof wherein:
Ring A is selected from the group consisting of C₆ aryl and 5-membered heteroaryl;

Ring B is

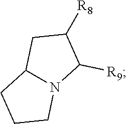

wherein variables in formula (I-a) are as defined in formula (I).

In some embodiments, the compound of formula (I), or a pharmaceutically acceptable salt thereof, is the compound of formula (I-a), or a pharmaceutically acceptable salt thereof m wherein: or the pharmaceutically acceptable salt thereof, in
Ring A is C₆ aryl.

In some embodiments, the compound of formula (I-a), is a pharmaceutically acceptable salt thereof, wherein:
ring A is 5-membered heteroaryl.

In certain aspects, the present disclosure provides a compound of formula (I-b),

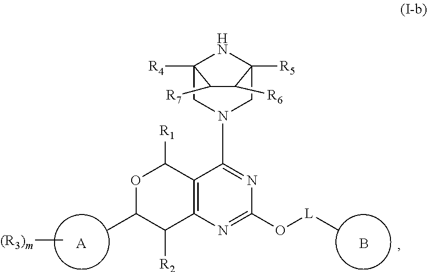

or a pharmaceutically acceptable salt thereof, wherein:
Ring A is selected from C₆ aryl and 5-membered heteroaryl;

Ring B is selected from

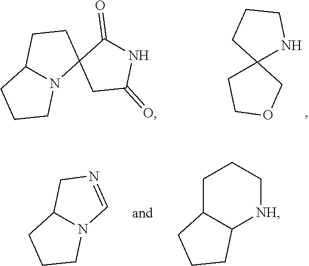 [SMILES: O=C1CC2(CCC3CCCN32)C(=O)N1 ; C1CNC2(C1)CCOC2 ; C1=NCC2CCCN12 ; C1CNC2CCCC2C1]

wherein the

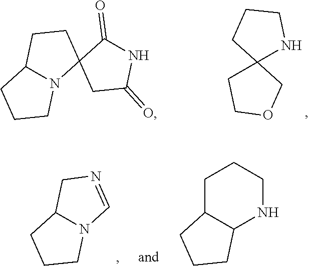 [SMILES: O=C1CC2(CCC3CCCN32)C(=O)N1 ; C1CNC2(C1)CCOC2 ; C1=NCC2CCCN12 ; C1CNC2CCCC2C1]

are each independently optionally substituted with 1, 2, 3, or 4 R₁₀;
wherein variables in formula (I-b) are as defined in formula (I).

In some embodiments, the compound of formula (I-b) above, or the pharmaceutically acceptable salt thereof, Ring A is C₆ aryl.

In some embodiments, the compound of formula (I-b), is a pharmaceutically acceptable salt thereof,
wherein ring A 5-membered heteroaryl.

In some embodiments, R₃ is independently selected from F, OH, NH₂, CF₃, OCH₃, —C≡CCH₃, —C≡CCH₂F, —C≡CCHF₂, —C≡CCF₃, —C≡CCD₃, —C≡CCl, —C≡CF and cyclopropyl, wherein other variables as defined in the present invention.

In some embodiments, ring A is phenyl.

In some embodiments,

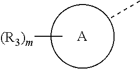

is selected from

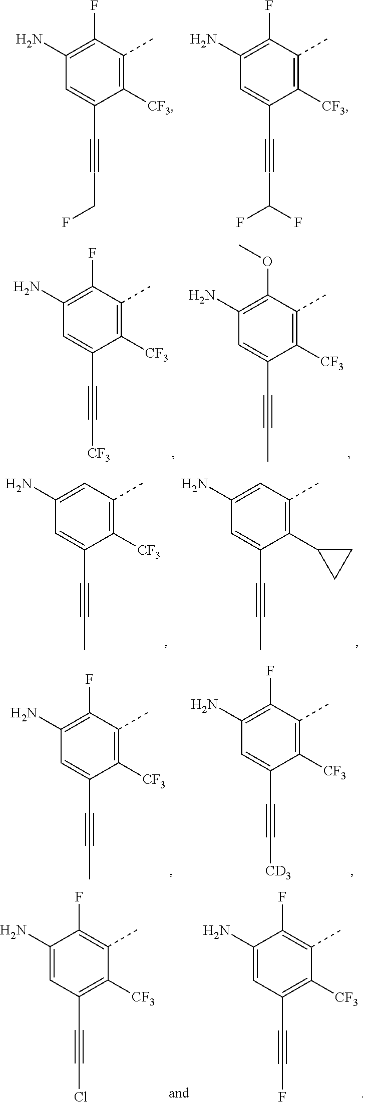

In some embodiments, R₅ is selected from H, OH, NH₂, CN, CH₃, CH₂CH₃, OCH₃, and O CH₂CH₃, wherein the H, OH, NH₂, CN, CH₃, CH₂CH₃, OCH₃, and OCH₂CH₃ are each independently optionally substituted with 1, 2, 3, 4, or 5b, as valency permits.

In some embodiments, R₅ is selected from H, CH₃, CH₂CN, CH₂OH and CH₂CONH₂.

In some embodiments, R₆ is selected from H and OH.

In some embodiments, R₄ is H.

In some embodiments, R₇ is H.

In some embodiments, R₈ is selected from H and F.

In some embodiments, R₉ is selected from —C(═O)—NH₂, —C(═O)—NHCH₃, —C(═O)—N(CH₃)₂ and —CH₂OH.

In some embodiments, R₁₀ is independently selected from D, F, NH₂, CH₃, —N(CH₃)₂, and —NH—C(═O)—CH₃.

In some embodiments, ring B is selected from

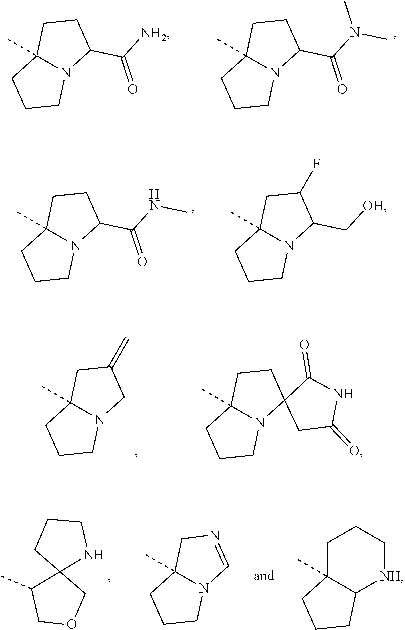

wherein the

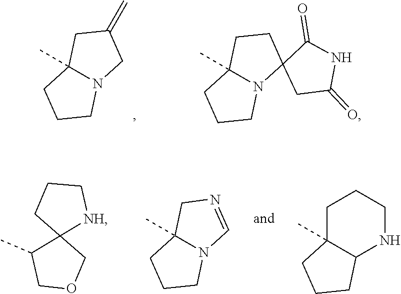

are each independently optionally substituted with 1, 2, 3, or 4 R₁₀.

In some embodiments, ring B is selected from

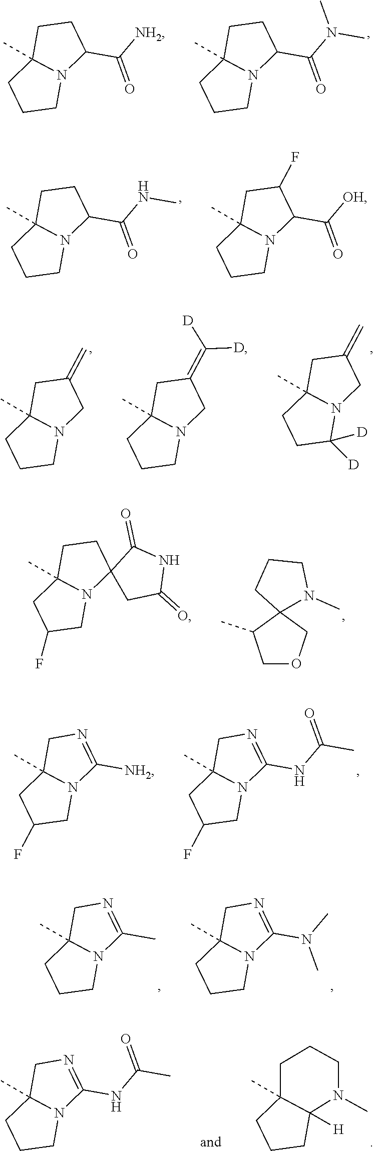

In some embodiments, the compound of Formula (I′) is a compound of Tables 1, 2, 2a, 3, 4, 5, 5a, 6, or 6a, a stereoisomer thereof, or a pharmaceutically acceptable salt thereof.

TABLE 1

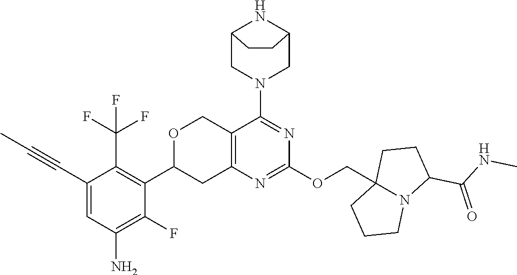 [SMILES: CC#Cc1cc(N)c(F)c(C2Cc3nc(OCC45CCCN4C(C(=O)NC)CC5)nc(N4CC5CCC(C4)N5)c3CO2)c1C(F)(F)F]

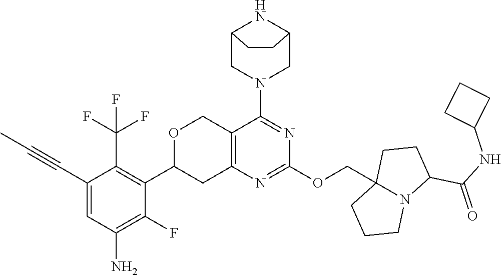 [SMILES: CC#Cc1cc(N)c(F)c(C2Cc3nc(OCC45CCCN4C(C(=O)NC4CCC4)CC5)nc(N4CC5CCC(C4)N5)c3CO2)c1C(F)(F)F]

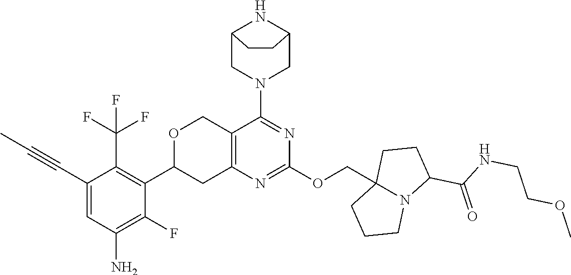 [SMILES: CC#Cc1cc(N)c(F)c(C2Cc3nc(OCC45CCCN4C(C(=O)NCCOC)CC5)nc(N4CC5CCC(C4)N5)c3CO2)c1C(F)(F)F]

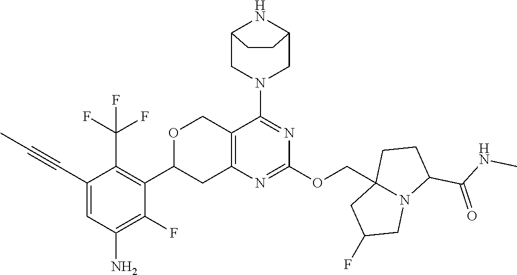 [SMILES: CC#Cc1cc(N)c(F)c(C2Cc3nc(OCC45CCC(C(=O)NC)N4CC(F)C5)nc(N4CC5CCC(C4)N5)c3CO2)c1C(F)(F)F]

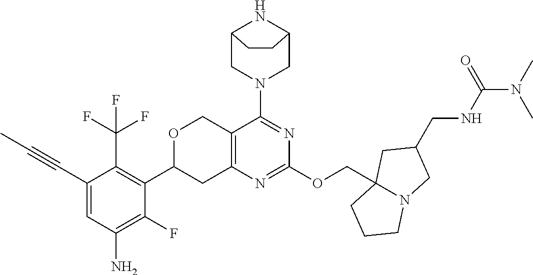 [SMILES: CC#Cc1cc(N)c(F)c(C2Cc3nc(OCC45CCCN4CC(CNC(=O)N(C)C)C5)nc(N4CC5CCC(C4)N5)c3CO2)c1C(F)(F)F]

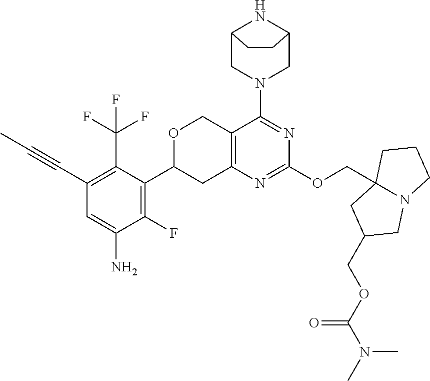 [SMILES: CC#Cc1cc(N)c(F)c(C2Cc3nc(OCC45CCCN4CC(COC(=O)N(C)C)C5)nc(N4CC5CCC(C4)N5)c3CO2)c1C(F)(F)F]

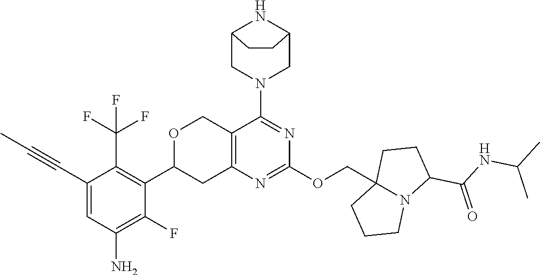 [SMILES: CC#Cc1cc(N)c(F)c(C2Cc3nc(OCC45CCCN4C(C(=O)NC(C)C)CC5)nc(N4CC5CCC(C4)N5)c3CO2)c1C(F)(F)F]

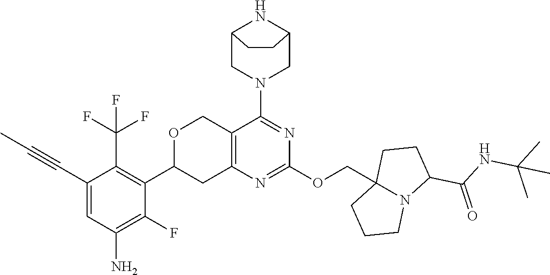 [SMILES: CC#Cc1cc(N)c(F)c(C2Cc3nc(OCC45CCCN4C(C(=O)NC(C)(C)C)CC5)nc(N4CC5CCC(C4)N5)c3CO2)c1C(F)(F)F]

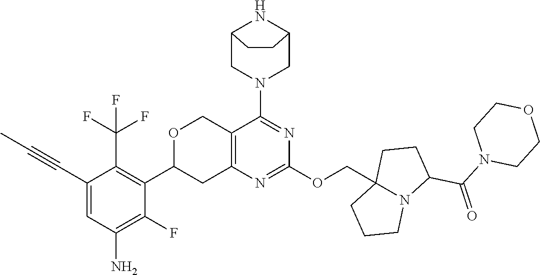 [SMILES: CC#Cc1cc(N)c(F)c(C2Cc3nc(OCC45CCCN4C(C(=O)N4CCOCC4)CC5)nc(N4CC5CCC(C4)N5)c3CO2)c1C(F)(F)F]

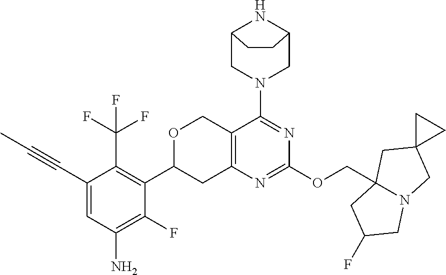 [SMILES: CC#Cc1cc(N)c(F)c(C2Cc3nc(OCC45CC(F)CN4CC4(CC4)C5)nc(N4CC5CCC(C4)N5)c3CO2)c1C(F)(F)F]

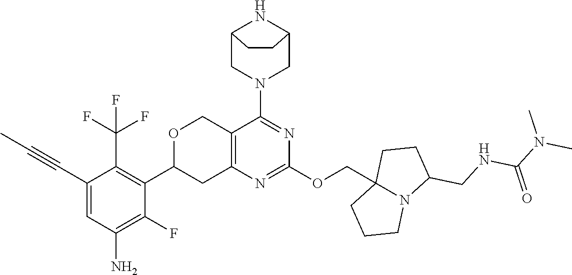 [SMILES: CC#Cc1cc(N)c(F)c(C2Cc3nc(OCC45CCCN4C(CNC(=O)N(C)C)CC5)nc(N4CC5CCC(C4)N5)c3CO2)c1C(F)(F)F]

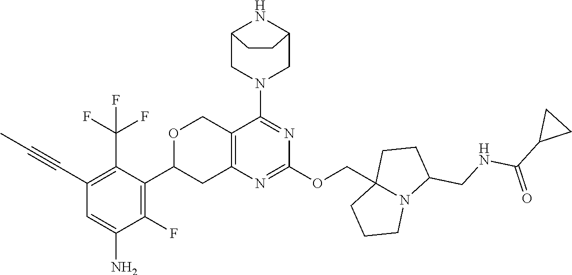 [SMILES: CC#Cc1cc(N)c(F)c(C2Cc3nc(OCC45CCCN4C(CNC(=O)C4CC4)CC5)nc(N4CC5CCC(C4)N5)c3CO2)c1C(F)(F)F]

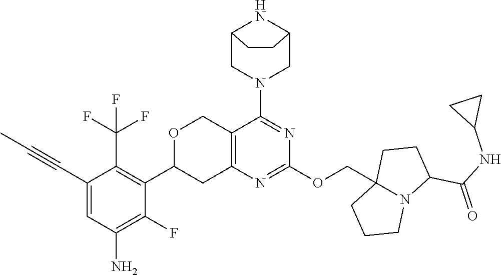 [SMILES: CC#Cc1cc(N)c(F)c(C2Cc3nc(OCC45CCCN4C(C(=O)NC4CC4)CC5)nc(N4CC5CCC(C4)N5)c3CO2)c1C(F)(F)F]

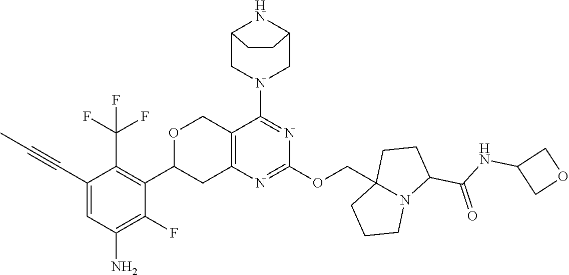 [SMILES: CC#Cc1cc(N)c(F)c(C2Cc3nc(OCC45CCCN4C(C(=O)NC4COC4)CC5)nc(N4CC5CCC(C4)N5)c3CO2)c1C(F)(F)F]

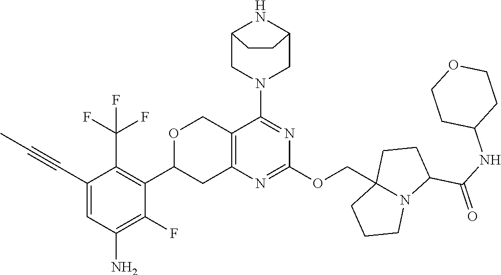 [SMILES: CC#Cc1cc(N)c(F)c(C2Cc3nc(OCC45CCCN4C(C(=O)NC4CCOCC4)CC5)nc(N4CC5CCC(C4)N5)c3CO2)c1C(F)(F)F]

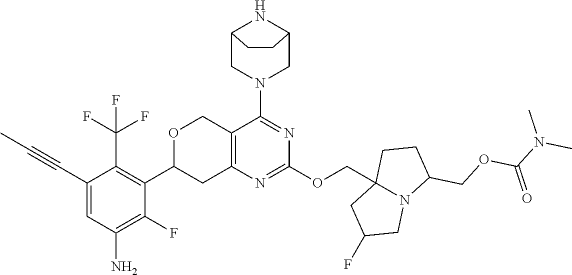 [SMILES: CC#Cc1cc(N)c(F)c(C2Cc3nc(OCC45CCC(COC(=O)N(C)C)N4CC(F)C5)nc(N4CC5CCC(C4)N5)c3CO2)c1C(F)(F)F]

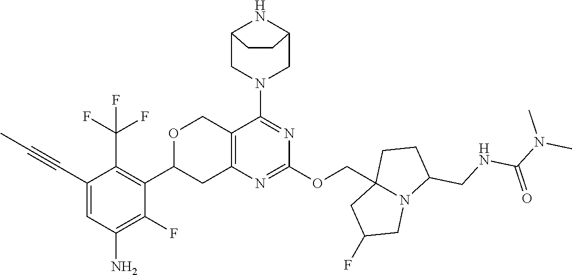 [SMILES: CC#Cc1cc(N)c(F)c(C2Cc3nc(OCC45CCC(CNC(=O)N(C)C)N4CC(F)C5)nc(N4CC5CCC(C4)N5)c3CO2)c1C(F)(F)F]

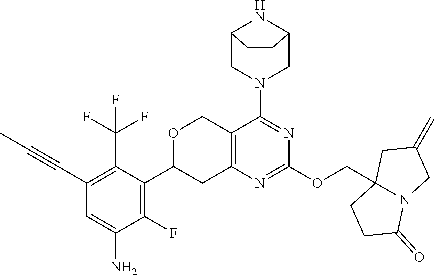 [SMILES: C=C1CN2C(=O)CCC2(COc2nc3c(c(N4CC5CCC(C4)N5)n2)COC(c2c(F)c(N)cc(C#CC)c2C(F)(F)F)C3)C1]

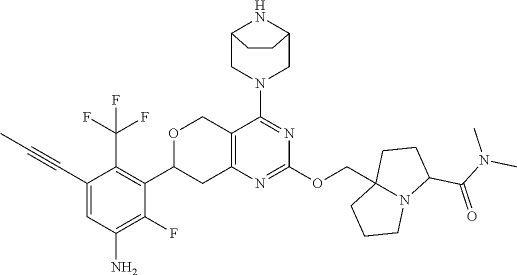 [SMILES: CC#Cc1cc(N)c(F)c(C2Cc3nc(OCC45CCCN4C(C(=O)N(C)C)CC5)nc(N4CC5CCC(C4)N5)c3CO2)c1C(F)(F)F]

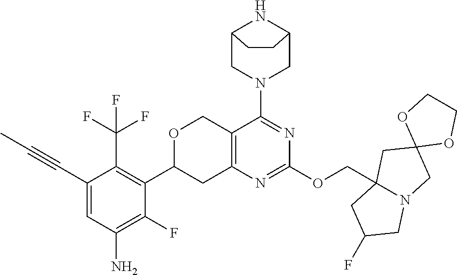 [SMILES: CC#Cc1cc(N)c(F)c(C2Cc3nc(OCC45CC(F)CN4CC4(C5)OCCO4)nc(N4CC5CCC(C4)N5)c3CO2)c1C(F)(F)F]

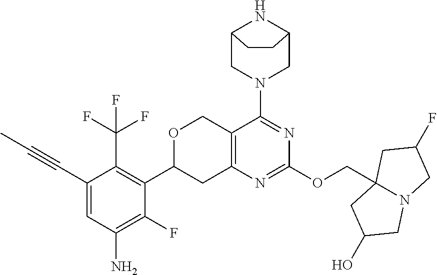 [SMILES: CC#Cc1cc(N)c(F)c(C2Cc3nc(OCC45CC(O)CN4CC(F)C5)nc(N4CC5CCC(C4)N5)c3CO2)c1C(F)(F)F]

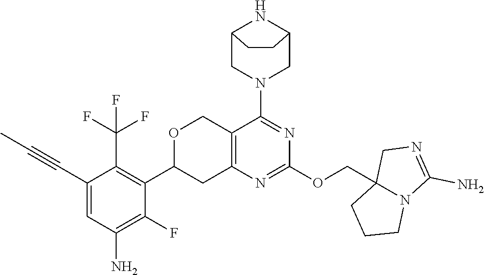 [SMILES: CC#Cc1cc(N)c(F)c(C2Cc3nc(OCC45CCCN4C(N)=NC5)nc(N4CC5CCC(C4)N5)c3CO2)c1C(F)(F)F]

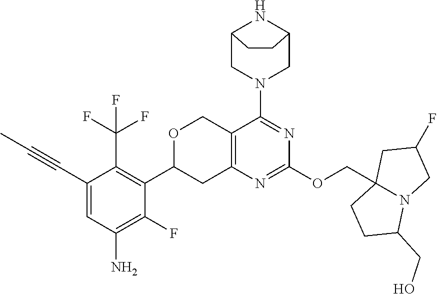 [SMILES: CC#Cc1cc(N)c(F)c(C2Cc3nc(OCC45CCC(CO)N4CC(F)C5)nc(N4CC5CCC(C4)N5)c3CO2)c1C(F)(F)F]

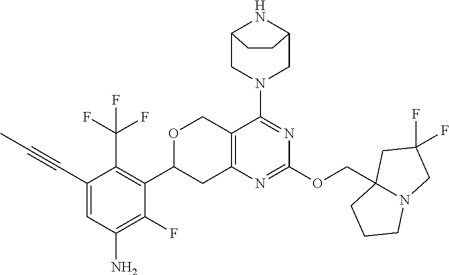 [SMILES: CC#Cc1cc(N)c(F)c(C2Cc3nc(OCC45CCCN4CC(F)(F)C5)nc(N4CC5CCC(C4)N5)c3CO2)c1C(F)(F)F]

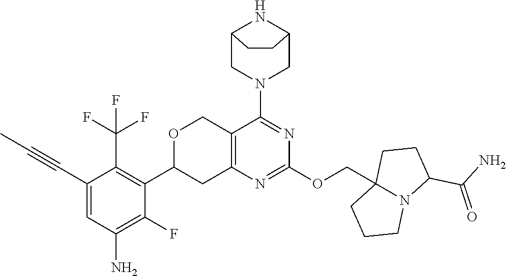 [SMILES: CC#Cc1cc(N)c(F)c(C2Cc3nc(OCC45CCCN4C(C(N)=O)CC5)nc(N4CC5CCC(C4)N5)c3CO2)c1C(F)(F)F]

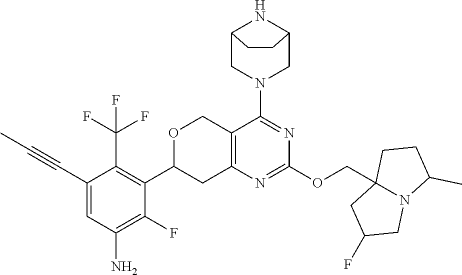 [SMILES: CC#Cc1cc(N)c(F)c(C2Cc3nc(OCC45CCC(C)N4CC(F)C5)nc(N4CC5CCC(C4)N5)c3CO2)c1C(F)(F)F]

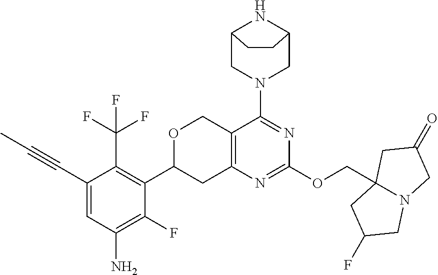 [SMILES: CC#Cc1cc(N)c(F)c(C2Cc3nc(OCC45CC(=O)CN4CC(F)C5)nc(N4CC5CCC(C4)N5)c3CO2)c1C(F)(F)F]

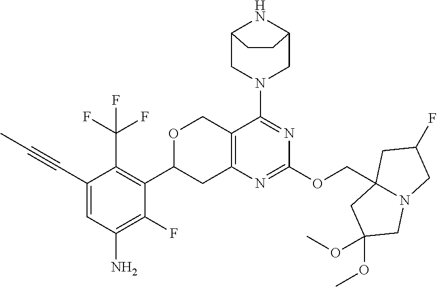 [SMILES: CC#Cc1cc(N)c(F)c(C2Cc3nc(OCC45CC(F)CN4CC(OC)(OC)C5)nc(N4CC5CCC(C4)N5)c3CO2)c1C(F)(F)F]

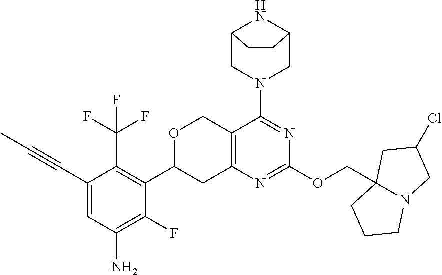 [SMILES: CC#Cc1cc(N)c(F)c(C2Cc3nc(OCC45CCCN4CC(Cl)C5)nc(N4CC5CCC(C4)N5)c3CO2)c1C(F)(F)F]

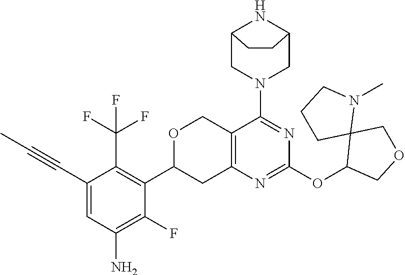 [SMILES: CC#Cc1cc(N)c(F)c(C2Cc3nc(OC4COCC45CCCN5C)nc(N4CC5CCC(C4)N5)c3CO2)c1C(F)(F)F]

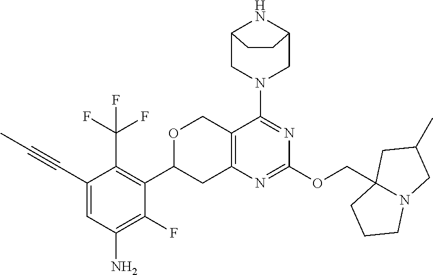 [SMILES: CC#Cc1cc(N)c(F)c(C2Cc3nc(OCC45CCCN4CC(C)C5)nc(N4CC5CCC(C4)N5)c3CO2)c1C(F)(F)F]

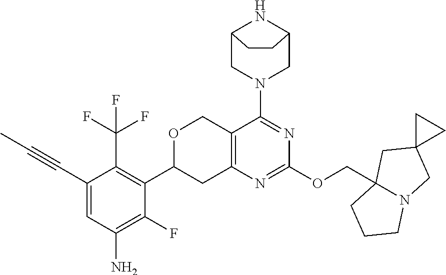 [SMILES: CC#Cc1cc(N)c(F)c(C2Cc3nc(OCC45CCCN4CC4(CC4)C5)nc(N4CC5CCC(C4)N5)c3CO2)c1C(F)(F)F]

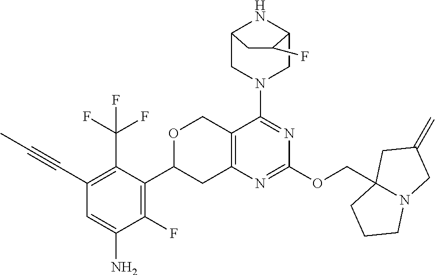 [SMILES: C=C1CN2CCCC2(COc2nc3c(c(N4CC5CC(F)C(C4)N5)n2)COC(c2c(F)c(N)cc(C#CC)c2C(F)(F)F)C3)C1]

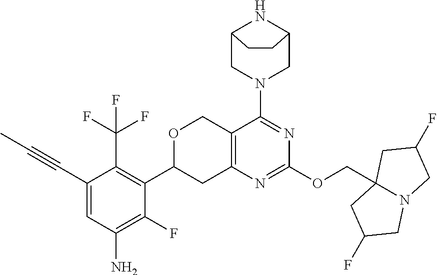 [SMILES: CC#Cc1cc(N)c(F)c(C2Cc3nc(OCC45CC(F)CN4CC(F)C5)nc(N4CC5CCC(C4)N5)c3CO2)c1C(F)(F)F]

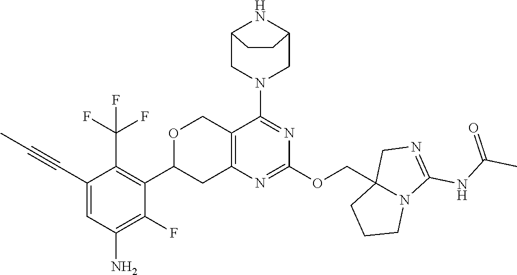 [SMILES: CC#Cc1cc(N)c(F)c(C2Cc3nc(OCC45CCCN4C(NC(C)=O)=NC5)nc(N4CC5CCC(C4)N5)c3CO2)c1C(F)(F)F]

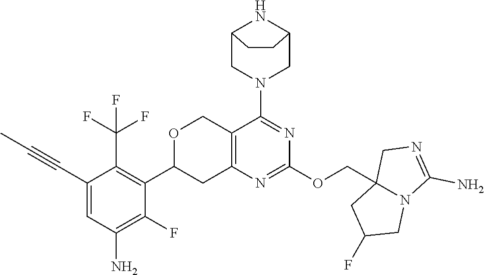 [SMILES: CC#Cc1cc(N)c(F)c(C2Cc3nc(OCC45CN=C(N)N4CC(F)C5)nc(N4CC5CCC(C4)N5)c3CO2)c1C(F)(F)F]

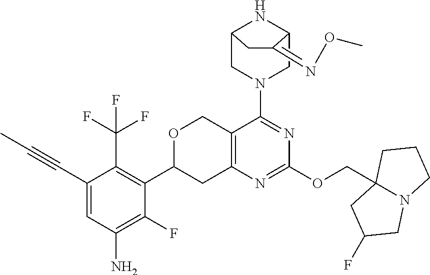 [SMILES: CC#Cc1cc(N)c(F)c(C2Cc3nc(OCC45CCCN4CC(F)C5)nc(N4CC5C/C(=N/OC)C(C4)N5)c3CO2)c1C(F)(F)F]

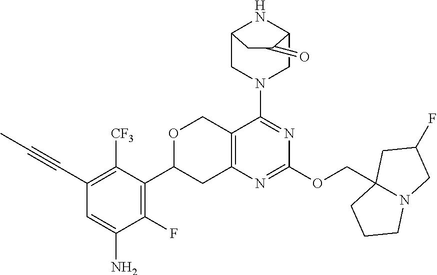 [SMILES: CC#Cc1cc(N)c(F)c(C2Cc3nc(OCC45CCCN4CC(F)C5)nc(N4CC5CC(=O)C(C4)N5)c3CO2)c1C(F)(F)F]

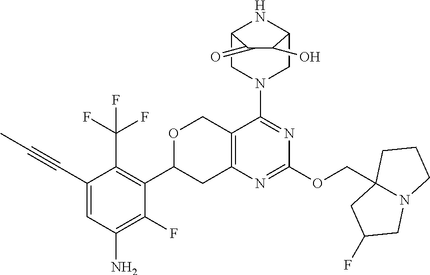 [SMILES: CC#Cc1cc(N)c(F)c(C2Cc3nc(OCC45CCCN4CC(F)C5)nc(N4CC5NC(C4)C(O)C5=O)c3CO2)c1C(F)(F)F]

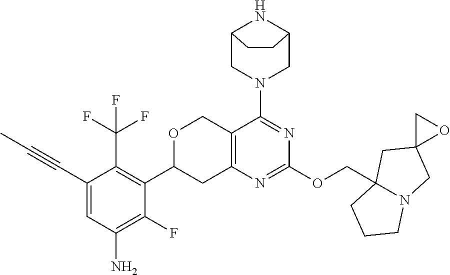 [SMILES: CC#Cc1cc(N)c(F)c(C2Cc3nc(OCC45CCCN4CC4(CO4)C5)nc(N4CC5CCC(C4)N5)c3CO2)c1C(F)(F)F]

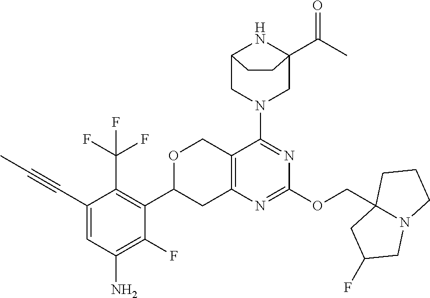 [SMILES: CC#Cc1cc(N)c(F)c(C2Cc3nc(OCC45CCCN4CC(F)C5)nc(N4CC5CCC(C(C)=O)(C4)N5)c3CO2)c1C(F)(F)F]

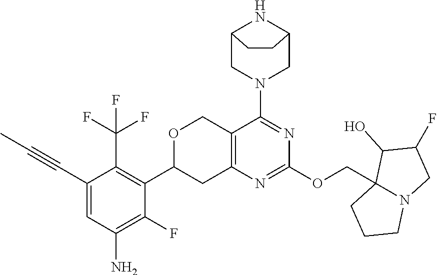 [SMILES: CC#Cc1cc(N)c(F)c(C2Cc3nc(OCC45CCCN4CC(F)C5O)nc(N4CC5CCC(C4)N5)c3CO2)c1C(F)(F)F]

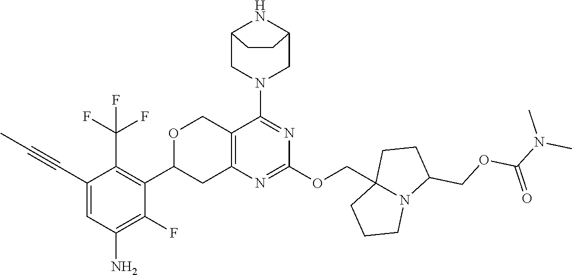 [SMILES: CC#Cc1cc(N)c(F)c(C2Cc3nc(OCC45CCCN4C(COC(=O)N(C)C)CC5)nc(N4CC5CCC(C4)N5)c3CO2)c1C(F)(F)F]

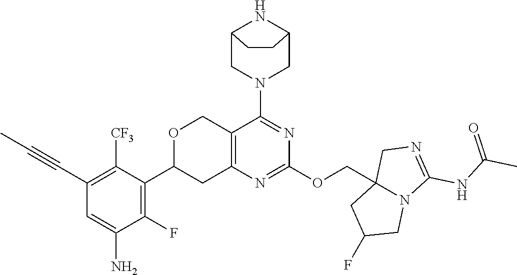 [SMILES: CC#Cc1cc(N)c(F)c(C2Cc3nc(OCC45CN=C(NC(C)=O)N4CC(F)C5)nc(N4CC5CCC(C4)N5)c3CO2)c1C(F)(F)F]

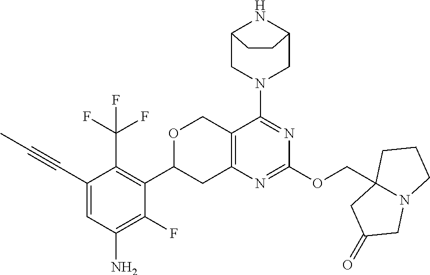 [SMILES: CC#Cc1cc(N)c(F)c(C2Cc3nc(OCC45CCCN4CC(=O)C5)nc(N4CC5CCC(C4)N5)c3CO2)c1C(F)(F)F]

—

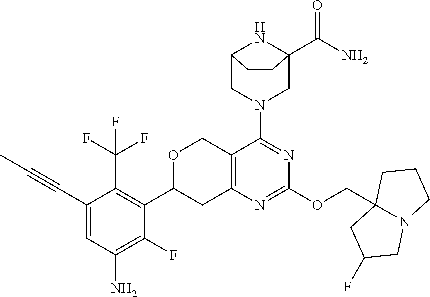 [SMILES: CC#Cc1cc(N)c(F)c(C2Cc3nc(OCC45CCCN4CC(F)C5)nc(N4CC5CCC(C(N)=O)(C4)N5)c3CO2)c1C(F)(F)F]

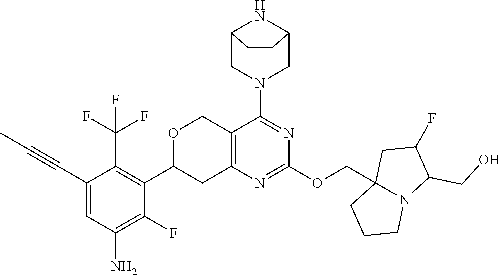 [SMILES: CC#Cc1cc(N)c(F)c(C2Cc3nc(OCC45CCCN4C(CO)C(F)C5)nc(N4CC5CCC(C4)N5)c3CO2)c1C(F)(F)F]

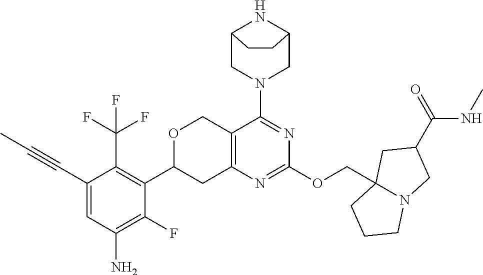 [SMILES: CC#Cc1cc(N)c(F)c(C2Cc3nc(OCC45CCCN4CC(C(=O)NC)C5)nc(N4CC5CCC(C4)N5)c3CO2)c1C(F)(F)F]

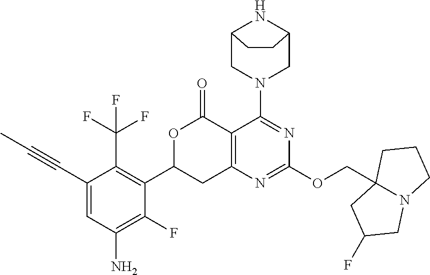 [SMILES: CC#Cc1cc(N)c(F)c(C2Cc3nc(OCC45CCCN4CC(F)C5)nc(N4CC5CCC(C4)N5)c3C(=O)O2)c1C(F)(F)F]

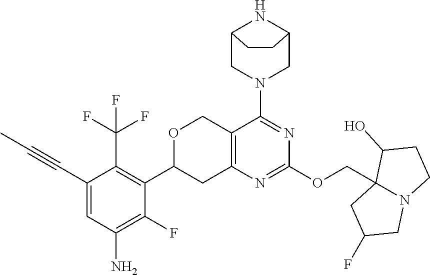 [SMILES: CC#Cc1cc(N)c(F)c(C2Cc3nc(OCC45CC(F)CN4CCC5O)nc(N4CC5CCC(C4)N5)c3CO2)c1C(F)(F)F]

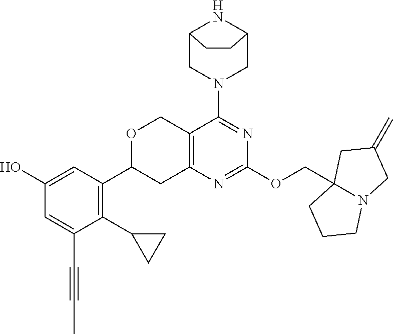 [SMILES: C=C1CN2CCCC2(COc2nc3c(c(N4CC5CCC(C4)N5)n2)COC(c2cc(O)cc(C#CC)c2C2CC2)C3)C1]

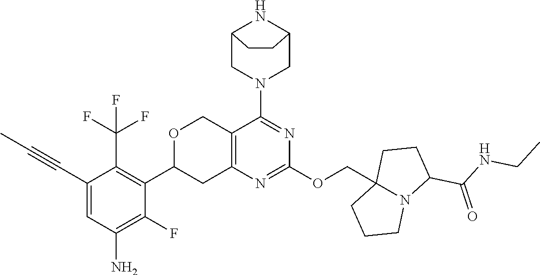 [SMILES: CC#Cc1cc(N)c(F)c(C2Cc3nc(OCC45CCCN4C(C(=O)NCC)CC5)nc(N4CC5CCC(C4)N5)c3CO2)c1C(F)(F)F]

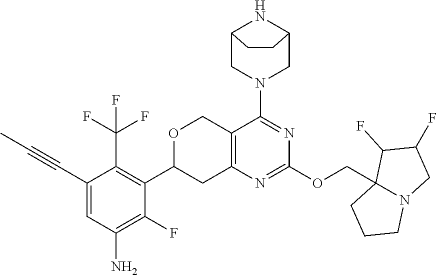 [SMILES: CC#Cc1cc(N)c(F)c(C2Cc3nc(OCC45CCCN4CC(F)C5F)nc(N4CC5CCC(C4)N5)c3CO2)c1C(F)(F)F]

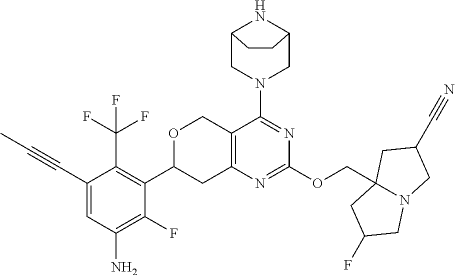 [SMILES: CC#Cc1cc(N)c(F)c(C2Cc3nc(OCC45CC(F)CN4CC(C#N)C5)nc(N4CC5CCC(C4)N5)c3CO2)c1C(F)(F)F]

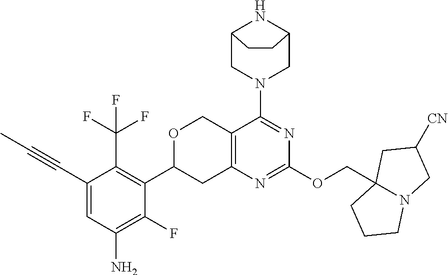 [SMILES: CC#Cc1cc(N)c(F)c(C2Cc3nc(OCC45CCCN4CC(C#N)C5)nc(N4CC5CCC(C4)N5)c3CO2)c1C(F)(F)F]

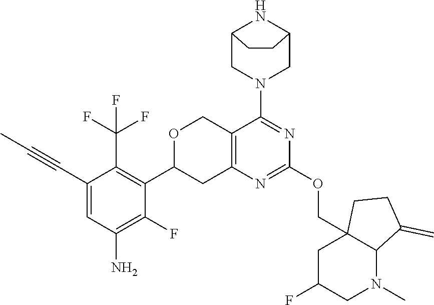 [SMILES: C=C1CCC2(COc3nc4c(c(N5CC6CCC(C5)N6)n3)COC(c3c(F)c(N)cc(C#CC)c3C(F)(F)F)C4)CC(F)CN(C)C12]

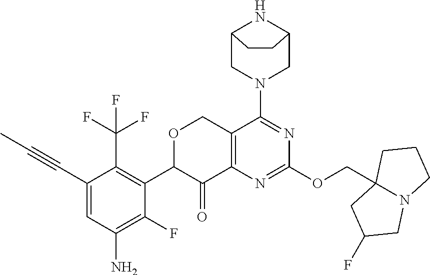 [SMILES: CC#Cc1cc(N)c(F)c(C2OCc3c(nc(OCC45CCCN4CC(F)C5)nc3N3CC4CCC(C3)N4)C2=O)c1C(F)(F)F]

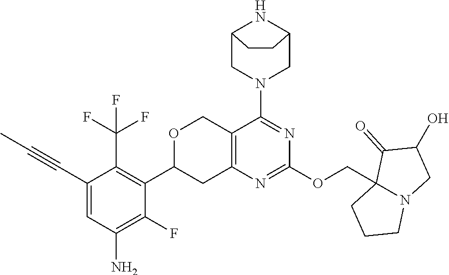 [SMILES: CC#Cc1cc(N)c(F)c(C2Cc3nc(OCC45CCCN4CC(O)C5=O)nc(N4CC5CCC(C4)N5)c3CO2)c1C(F)(F)F]

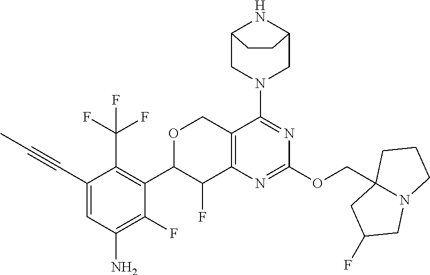 [SMILES: CC#Cc1cc(N)c(F)c(C2OCc3c(nc(OCC45CCCN4CC(F)C5)nc3N3CC4CCC(C3)N4)C2F)c1C(F)(F)F]

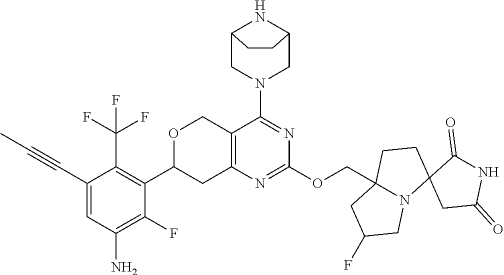 [SMILES: CC#Cc1cc(N)c(F)c(C2Cc3nc(OCC45CCC6(CC(=O)NC6=O)N4CC(F)C5)nc(N4CC5CCC(C4)N5)c3CO2)c1C(F)(F)F]

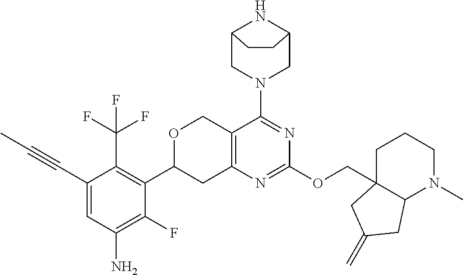 [SMILES: C=C1CC2N(C)CCCC2(COc2nc3c(c(N4CC5CCC(C4)N5)n2)COC(c2c(F)c(N)cc(C#CC)c2C(F)(F)F)C3)C1]

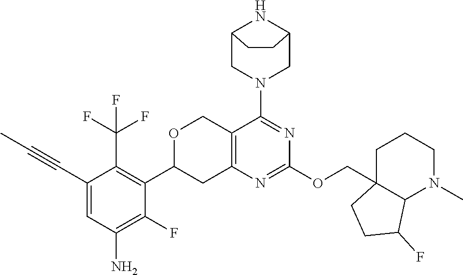 [SMILES: CC#Cc1cc(N)c(F)c(C2Cc3nc(OCC45CCCN(C)C4C(F)CC5)nc(N4CC5CCC(C4)N5)c3CO2)c1C(F)(F)F]

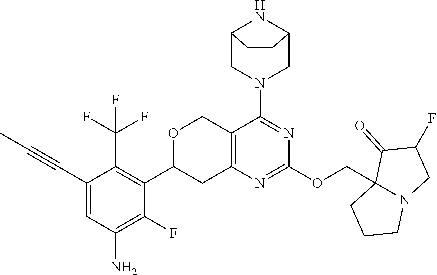 [SMILES: CC#Cc1cc(N)c(F)c(C2Cc3nc(OCC45CCCN4CC(F)C5=O)nc(N4CC5CCC(C4)N5)c3CO2)c1C(F)(F)F]

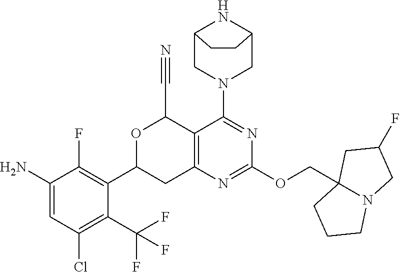 [SMILES: N#CC1OC(c2c(F)c(N)cc(Cl)c2C(F)(F)F)Cc2nc(OCC34CCCN3CC(F)C4)nc(N3CC4CCC(C3)N4)c21]

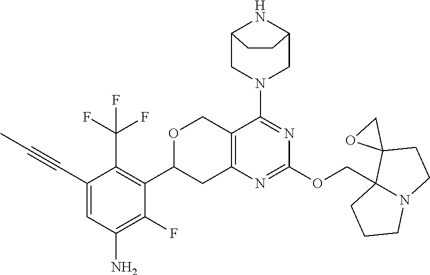 [SMILES: CC#Cc1cc(N)c(F)c(C2Cc3nc(OCC45CCCN4CCC54CO4)nc(N4CC5CCC(C4)N5)c3CO2)c1C(F)(F)F]

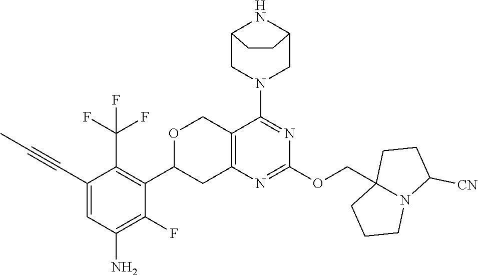 [SMILES: CC#Cc1cc(N)c(F)c(C2Cc3nc(OCC45CCCN4C(C#N)CC5)nc(N4CC5CCC(C4)N5)c3CO2)c1C(F)(F)F]

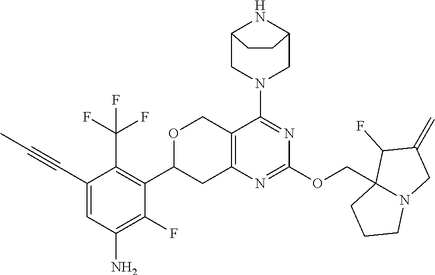 [SMILES: C=C1CN2CCCC2(COc2nc3c(c(N4CC5CCC(C4)N5)n2)COC(c2c(F)c(N)cc(C#CC)c2C(F)(F)F)C3)C1F]

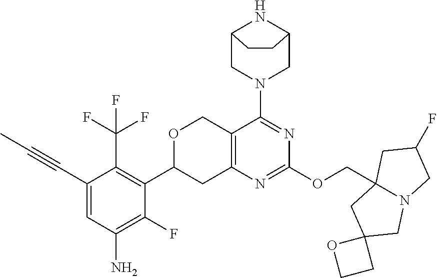 [SMILES: CC#Cc1cc(N)c(F)c(C2Cc3nc(OCC45CC(F)CN4CC4(CCO4)C5)nc(N4CC5CCC(C4)N5)c3CO2)c1C(F)(F)F]

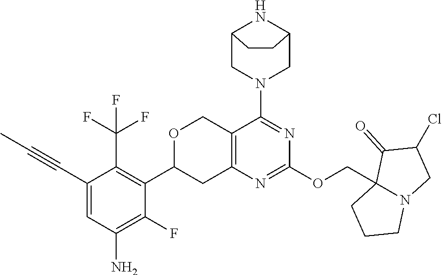 [SMILES: CC#Cc1cc(N)c(F)c(C2Cc3nc(OCC45CCCN4CC(Cl)C5=O)nc(N4CC5CCC(C4)N5)c3CO2)c1C(F)(F)F]

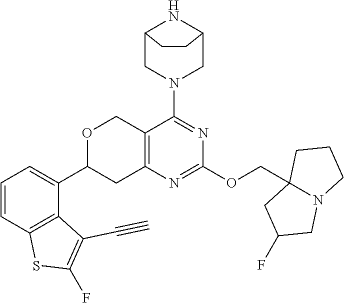 [SMILES: C#Cc1c(F)sc2cccc(C3Cc4nc(OCC56CCCN5CC(F)C6)nc(N5CC6CCC(C5)N6)c4CO3)c12]

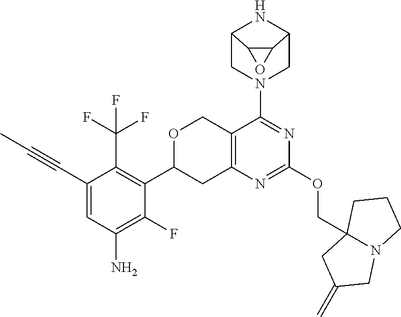 [SMILES: C=C1CN2CCCC2(COc2nc3c(c(N4CC5NC(C4)C4OC54)n2)COC(c2c(F)c(N)cc(C#CC)c2C(F)(F)F)C3)C1]

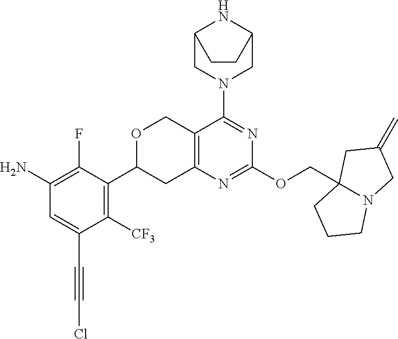 [SMILES: C=C1CN2CCCC2(COc2nc3c(c(N4CC5CCC(C4)N5)n2)COC(c2c(F)c(N)cc(C#CCl)c2C(F)(F)F)C3)C1]

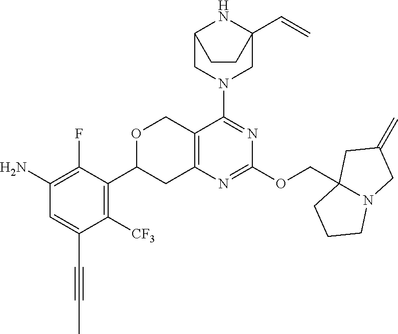 [SMILES: C=CC12CCC(CN(c3nc(OCC45CCCN4CC(=C)C5)nc4c3COC(c3c(F)c(N)cc(C#CC)c3C(F)(F)F)C4)C1)N2]

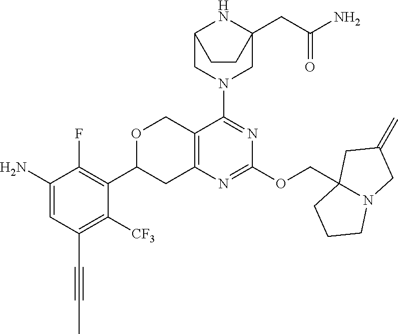 [SMILES: C=C1CN2CCCC2(COc2nc3c(c(N4CC5CCC(CC(N)=O)(C4)N5)n2)COC(c2c(F)c(N)cc(C#CC)c2C(F)(F)F)C3)C1]

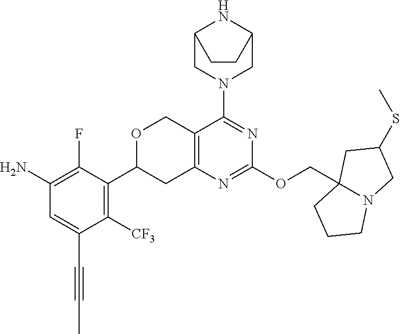 [SMILES: CC#Cc1cc(N)c(F)c(C2Cc3nc(OCC45CCCN4CC(SC)C5)nc(N4CC5CCC(C4)N5)c3CO2)c1C(F)(F)F]

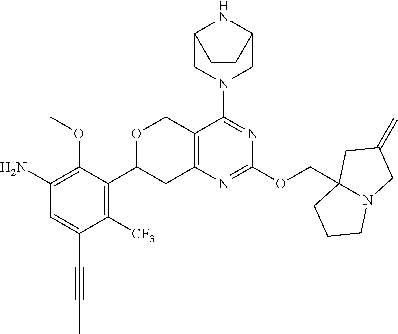 [SMILES: C=C1CN2CCCC2(COc2nc3c(c(N4CC5CCC(C4)N5)n2)COC(c2c(OC)c(N)cc(C#CC)c2C(F)(F)F)C3)C1]

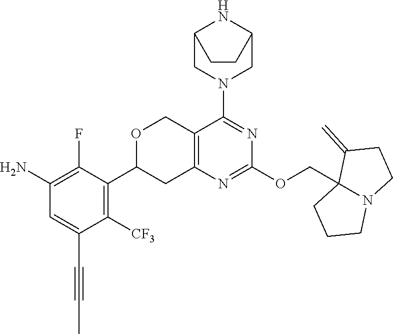 [SMILES: C=C1CCN2CCCC12COc1nc2c(c(N3CC4CCC(C3)N4)n1)COC(c1c(F)c(N)cc(C#CC)c1C(F)(F)F)C2]

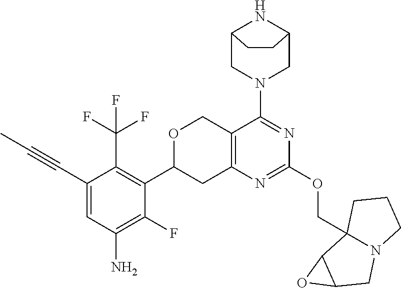 [SMILES: CC#Cc1cc(N)c(F)c(C2Cc3nc(OCC45CCCN4CC4OC45)nc(N4CC5CCC(C4)N5)c3CO2)c1C(F)(F)F]

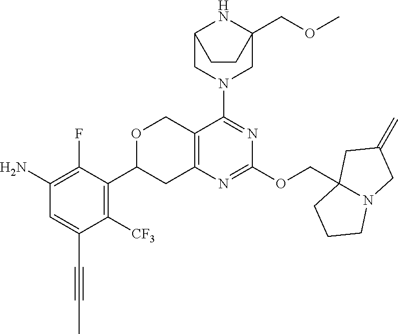 [SMILES: C=C1CN2CCCC2(COc2nc3c(c(N4CC5CCC(COC)(C4)N5)n2)COC(c2c(F)c(N)cc(C#CC)c2C(F)(F)F)C3)C1]

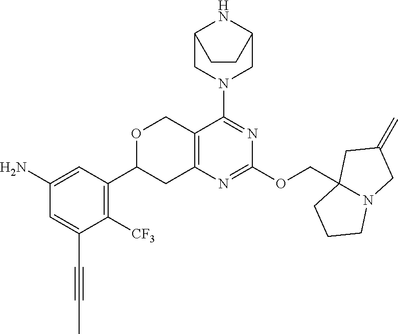 [SMILES: C=C1CN2CCCC2(COc2nc3c(c(N4CC5CCC(C4)N5)n2)COC(c2cc(N)cc(C#CC)c2C(F)(F)F)C3)C1]

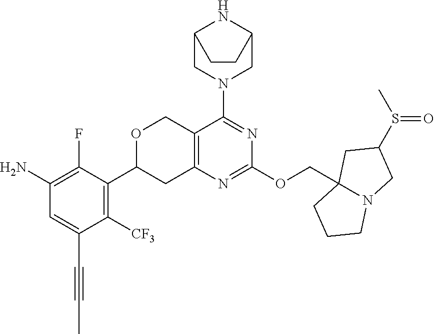 [SMILES: CC#Cc1cc(N)c(F)c(C2Cc3nc(OCC45CCCN4CC(S(C)=O)C5)nc(N4CC5CCC(C4)N5)c3CO2)c1C(F)(F)F]

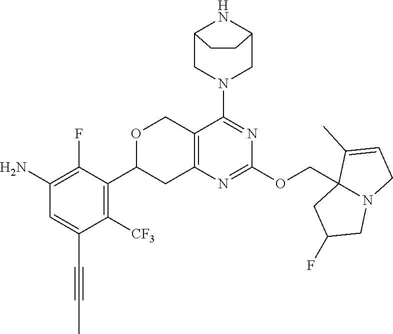 [SMILES: CC#Cc1cc(N)c(F)c(C2Cc3nc(OCC45CC(F)CN4CC=C5C)nc(N4CC5CCC(C4)N5)c3CO2)c1C(F)(F)F]

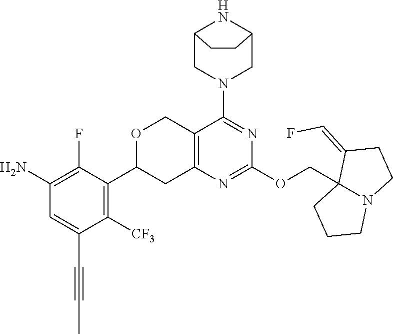 [SMILES: CC#Cc1cc(N)c(F)c(C2Cc3nc(OCC45CCCN4CC/C5=C/F)nc(N4CC5CCC(C4)N5)c3CO2)c1C(F)(F)F]

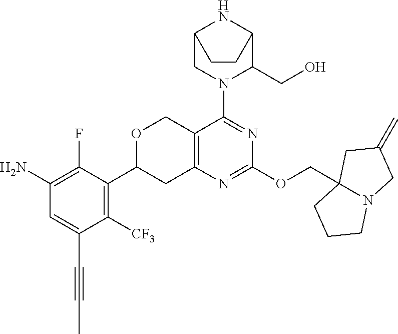 [SMILES: C=C1CN2CCCC2(COc2nc3c(c(N4CC5CCC(N5)C4CO)n2)COC(c2c(F)c(N)cc(C#CC)c2C(F)(F)F)C3)C1]

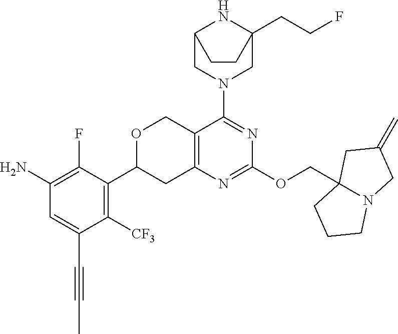 [SMILES: C=C1CN2CCCC2(COc2nc3c(c(N4CC5CCC(CCF)(C4)N5)n2)COC(c2c(F)c(N)cc(C#CC)c2C(F)(F)F)C3)C1]

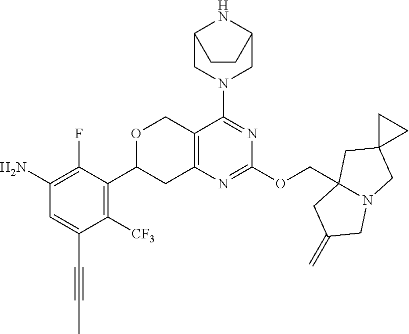 [SMILES: C=C1CN2CC3(CC3)CC2(COc2nc3c(c(N4CC5CCC(C4)N5)n2)COC(c2c(F)c(N)cc(C#CC)c2C(F)(F)F)C3)C1]

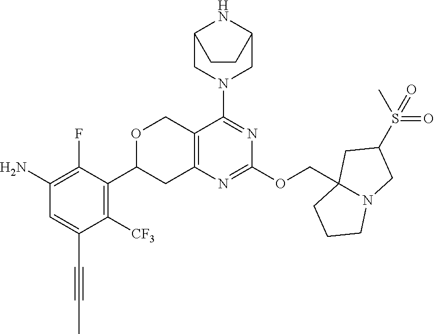 [SMILES: CC#Cc1cc(N)c(F)c(C2Cc3nc(OCC45CCCN4CC(S(C)(=O)=O)C5)nc(N4CC5CCC(C4)N5)c3CO2)c1C(F)(F)F]

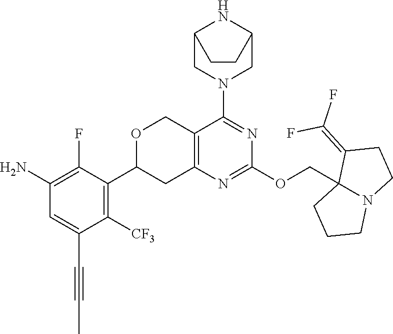 [SMILES: CC#Cc1cc(N)c(F)c(C2Cc3nc(OCC45CCCN4CCC5=C(F)F)nc(N4CC5CCC(C4)N5)c3CO2)c1C(F)(F)F]

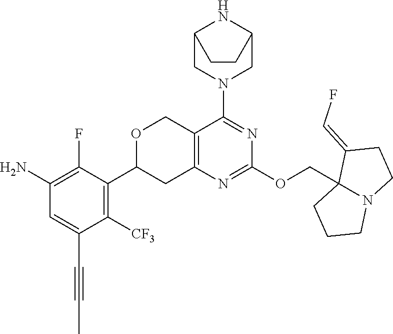 [SMILES: CC#Cc1cc(N)c(F)c(C2Cc3nc(OCC45CCCN4CC/C5=C\F)nc(N4CC5CCC(C4)N5)c3CO2)c1C(F)(F)F]

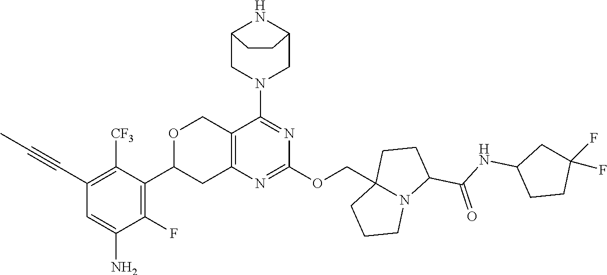 [SMILES: CC#Cc1cc(N)c(F)c(C2Cc3nc(OCC45CCCN4C(C(=O)NC4CCC(F)(F)C4)CC5)nc(N4CC5CCC(C4)N5)c3CO2)c1C(F)(F)F]

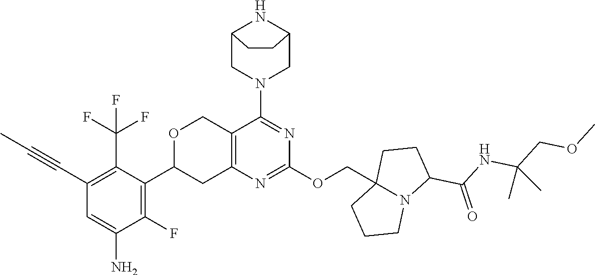 [SMILES: CC#Cc1cc(N)c(F)c(C2Cc3nc(OCC45CCCN4C(C(=O)NC(C)(C)COC)CC5)nc(N4CC5CCC(C4)N5)c3CO2)c1C(F)(F)F]

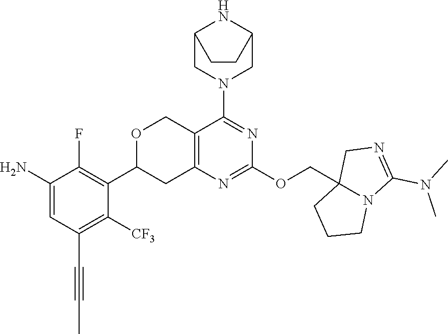 [SMILES: CC#Cc1cc(N)c(F)c(C2Cc3nc(OCC45CCCN4C(N(C)C)=NC5)nc(N4CC5CCC(C4)N5)c3CO2)c1C(F)(F)F]

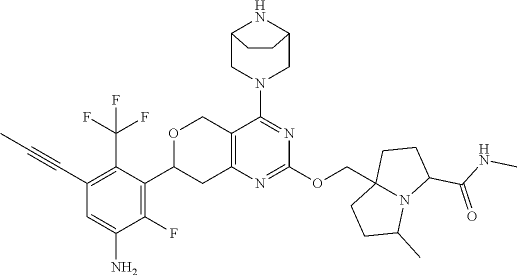 [SMILES: CC#Cc1cc(N)c(F)c(C2Cc3nc(OCC45CCC(C)N4C(C(=O)NC)CC5)nc(N4CC5CCC(C4)N5)c3CO2)c1C(F)(F)F]

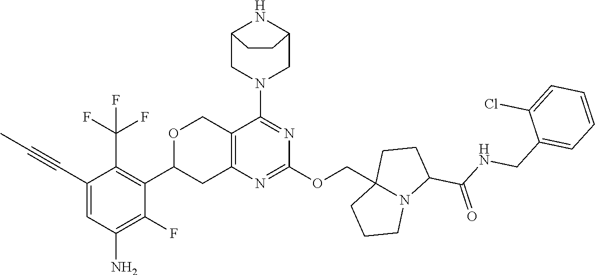 [SMILES: CC#Cc1cc(N)c(F)c(C2Cc3nc(OCC45CCCN4C(C(=O)NCc4ccccc4Cl)CC5)nc(N4CC5CCC(C4)N5)c3CO2)c1C(F)(F)F]

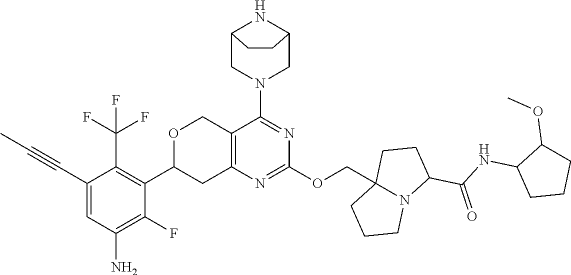 [SMILES: CC#Cc1cc(N)c(F)c(C2Cc3nc(OCC45CCCN4C(C(=O)NC4CCCC4OC)CC5)nc(N4CC5CCC(C4)N5)c3CO2)c1C(F)(F)F]

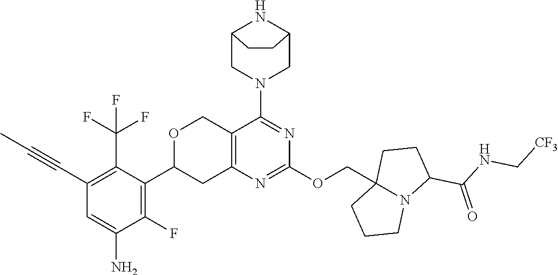 [SMILES: CC#Cc1cc(N)c(F)c(C2Cc3nc(OCC45CCCN4C(C(=O)NCC(F)(F)F)CC5)nc(N4CC5CCC(C4)N5)c3CO2)c1C(F)(F)F]

—

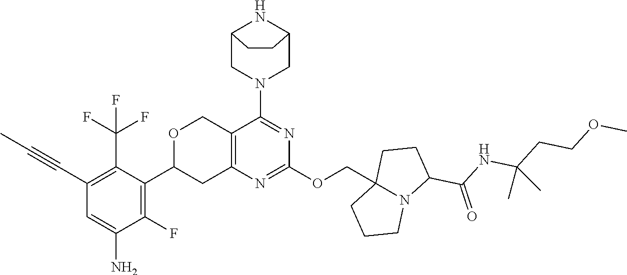 [SMILES: CC#Cc1cc(N)c(F)c(C2Cc3nc(OCC45CCCN4C(C(=O)NC(C)(C)CCOC)CC5)nc(N4CC5CCC(C4)N5)c3CO2)c1C(F)(F)F]

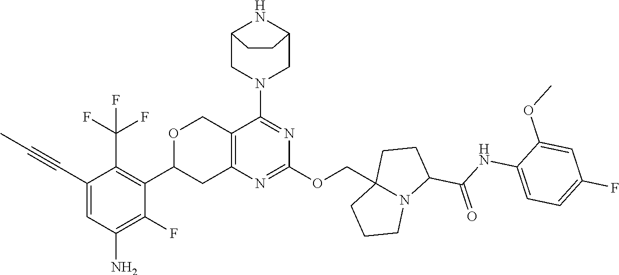 [SMILES: CC#Cc1cc(N)c(F)c(C2Cc3nc(OCC45CCCN4C(C(=O)Nc4ccc(F)cc4OC)CC5)nc(N4CC5CCC(C4)N5)c3CO2)c1C(F)(F)F]

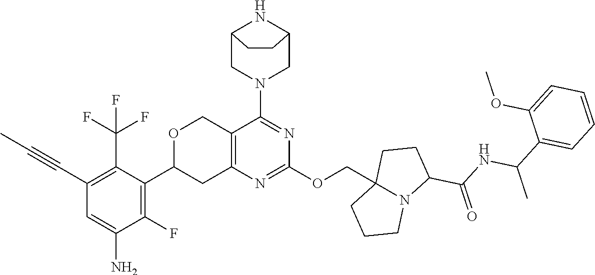 [SMILES: CC#Cc1cc(N)c(F)c(C2Cc3nc(OCC45CCCN4C(C(=O)NC(C)c4ccccc4OC)CC5)nc(N4CC5CCC(C4)N5)c3CO2)c1C(F)(F)F]

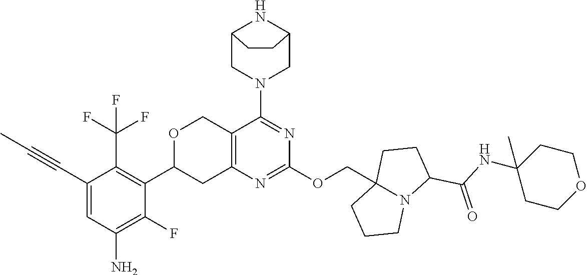 [SMILES: CC#Cc1cc(N)c(F)c(C2Cc3nc(OCC45CCCN4C(C(=O)NC4(C)CCOCC4)CC5)nc(N4CC5CCC(C4)N5)c3CO2)c1C(F)(F)F]

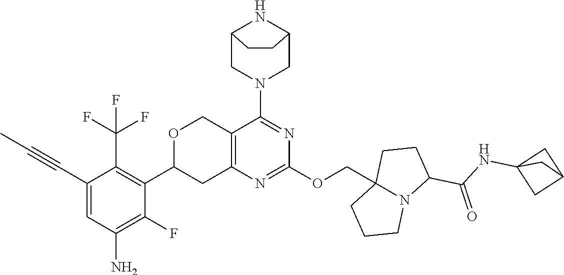 [SMILES: CC#Cc1cc(N)c(F)c(C2Cc3nc(OCC45CCCN4C(C(=O)NC46CC(C4)C6)CC5)nc(N4CC5CCC(C4)N5)c3CO2)c1C(F)(F)F]

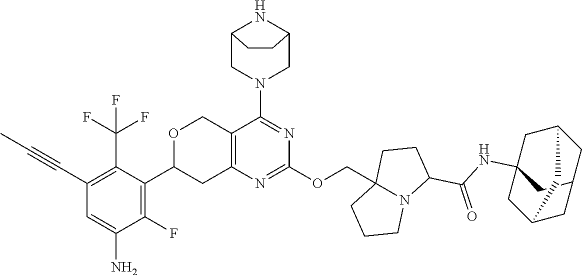 [SMILES: CC#Cc1cc(N)c(F)c(C2Cc3nc(OCC45CCCN4C(C(=O)N[C@]46CC7C[C@H](C[C@H](C7)C4)C6)CC5)nc(N4CC5CCC(C4)N5)c3CO2)c1C(F)(F)F]

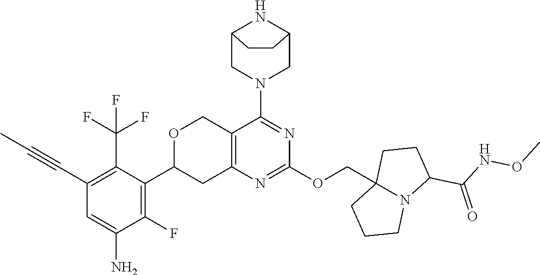 [SMILES: CC#Cc1cc(N)c(F)c(C2Cc3nc(OCC45CCCN4C(C(=O)NOC)CC5)nc(N4CC5CCC(C4)N5)c3CO2)c1C(F)(F)F]

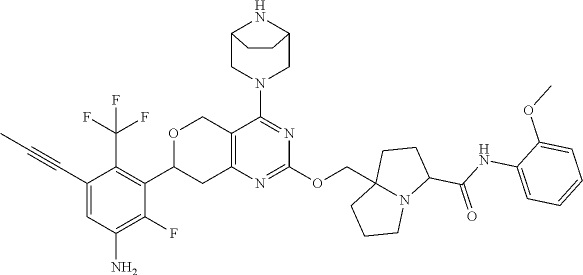 [SMILES: CC#Cc1cc(N)c(F)c(C2Cc3nc(OCC45CCCN4C(C(=O)Nc4ccccc4OC)CC5)nc(N4CC5CCC(C4)N5)c3CO2)c1C(F)(F)F]

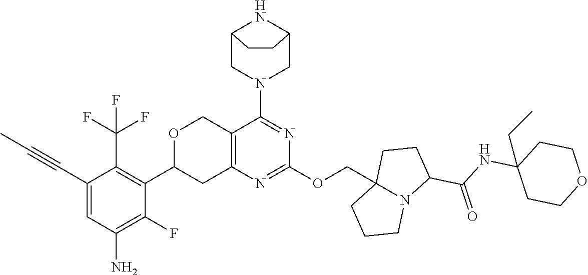 [SMILES: CC#Cc1cc(N)c(F)c(C2Cc3nc(OCC45CCCN4C(C(=O)NC4(CC)CCOCC4)CC5)nc(N4CC5CCC(C4)N5)c3CO2)c1C(F)(F)F]

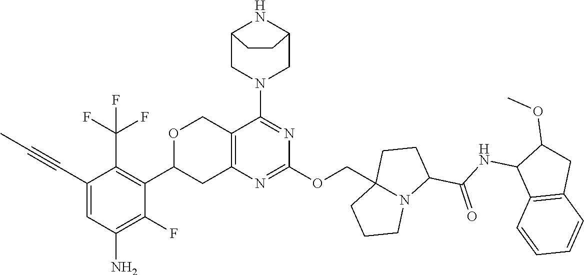 [SMILES: CC#Cc1cc(N)c(F)c(C2Cc3nc(OCC45CCCN4C(C(=O)NC4c6ccccc6CC4OC)CC5)nc(N4CC5CCC(C4)N5)c3CO2)c1C(F)(F)F]

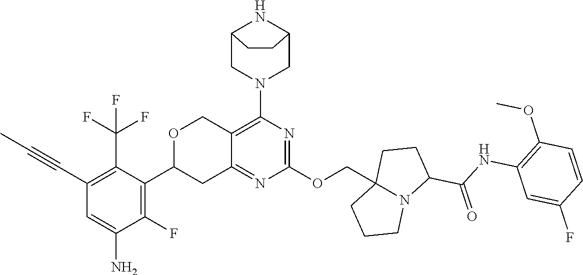 [SMILES: CC#Cc1cc(N)c(F)c(C2Cc3nc(OCC45CCCN4C(C(=O)Nc4cc(F)ccc4OC)CC5)nc(N4CC5CCC(C4)N5)c3CO2)c1C(F)(F)F]

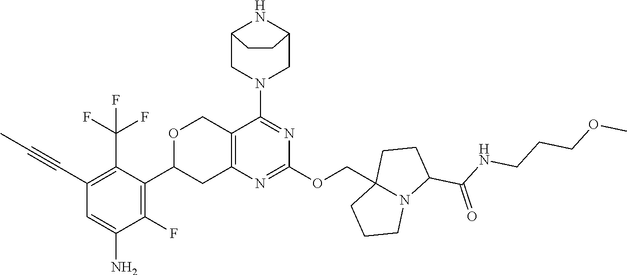 [SMILES: CC#Cc1cc(N)c(F)c(C2Cc3nc(OCC45CCCN4C(C(=O)NCCCOC)CC5)nc(N4CC5CCC(C4)N5)c3CO2)c1C(F)(F)F]

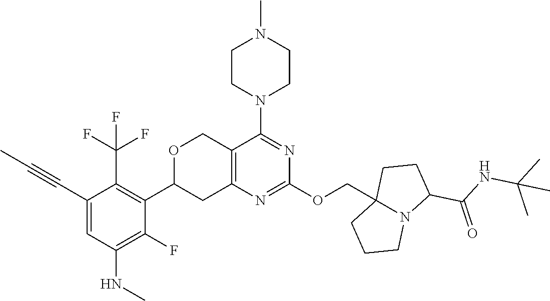 [SMILES: CC#Cc1cc(NC)c(F)c(C2Cc3nc(OCC45CCCN4C(C(=O)NC(C)(C)C)CC5)nc(N4CCN(C)CC4)c3CO2)c1C(F)(F)F]

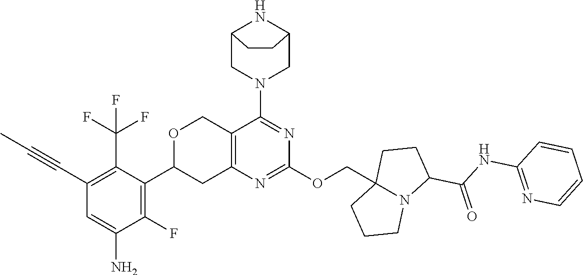 [SMILES: CC#Cc1cc(N)c(F)c(C2Cc3nc(OCC45CCCN4C(C(=O)Nc4ccccn4)CC5)nc(N4CC5CCC(C4)N5)c3CO2)c1C(F)(F)F]

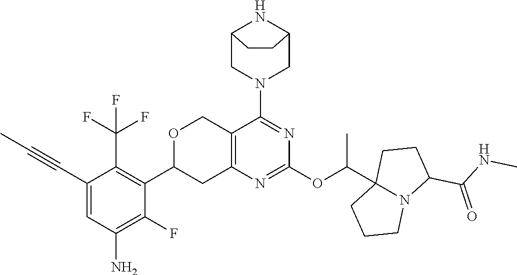 [SMILES: CC#Cc1cc(N)c(F)c(C2Cc3nc(OC(C)C45CCCN4C(C(=O)NC)CC5)nc(N4CC5CCC(C4)N5)c3CO2)c1C(F)(F)F]

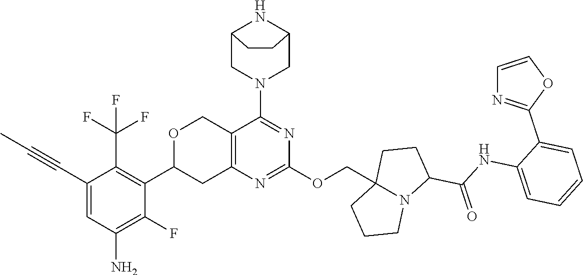 [SMILES: CC#Cc1cc(N)c(F)c(C2Cc3nc(OCC45CCCN4C(C(=O)Nc4ccccc4-c4ncco4)CC5)nc(N4CC5CCC(C4)N5)c3CO2)c1C(F)(F)F]

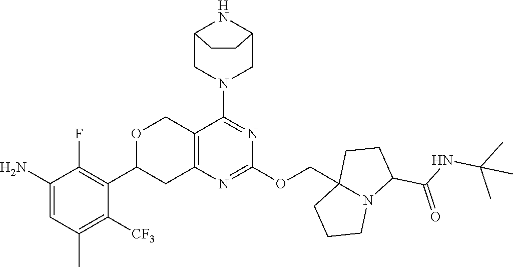 [SMILES: Cc1cc(N)c(F)c(C2Cc3nc(OCC45CCCN4C(C(=O)NC(C)(C)C)CC5)nc(N4CC5CCC(C4)N5)c3CO2)c1C(F)(F)F]

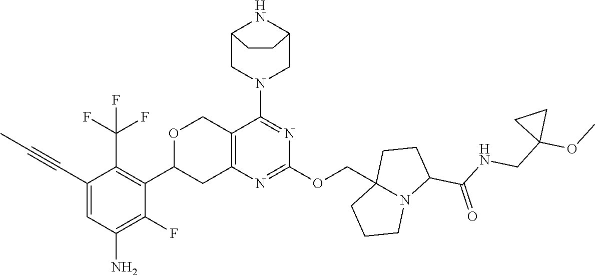 [SMILES: CC#Cc1cc(N)c(F)c(C2Cc3nc(OCC45CCCN4C(C(=O)NCC4(OC)CC4)CC5)nc(N4CC5CCC(C4)N5)c3CO2)c1C(F)(F)F]

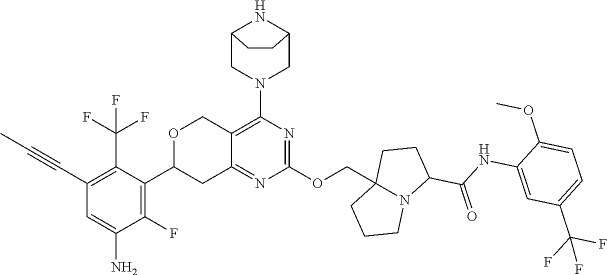 [SMILES: CC#Cc1cc(N)c(F)c(C2Cc3nc(OCC45CCCN4C(C(=O)Nc4cc(C(F)(F)F)ccc4OC)CC5)nc(N4CC5CCC(C4)N5)c3CO2)c1C(F)(F)F]

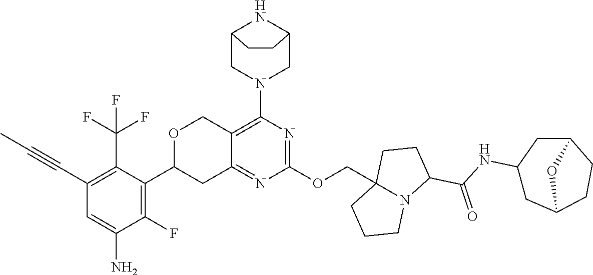 [SMILES: CC#Cc1cc(N)c(F)c(C2Cc3nc(OCC45CCCN4C(C(=O)NC4C[C@H]6CC[C@@H](C4)O6)CC5)nc(N4CC5CCC(C4)N5)c3CO2)c1C(F)(F)F]

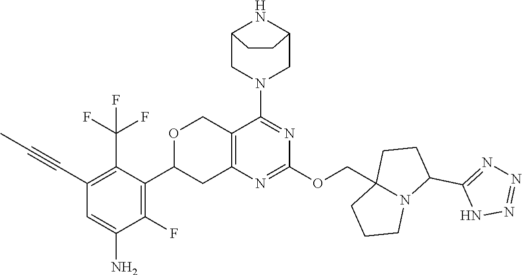 [SMILES: CC#Cc1cc(N)c(F)c(C2Cc3nc(OCC45CCCN4C(c4nnn[nH]4)CC5)nc(N4CC5CCC(C4)N5)c3CO2)c1C(F)(F)F]

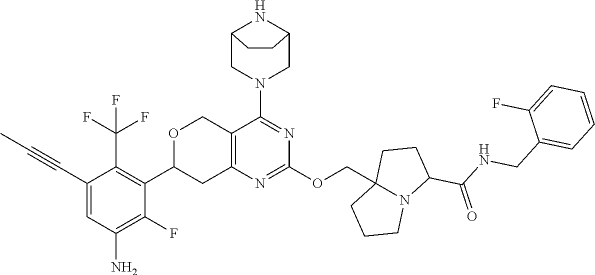 [SMILES: CC#Cc1cc(N)c(F)c(C2Cc3nc(OCC45CCCN4C(C(=O)NCc4ccccc4F)CC5)nc(N4CC5CCC(C4)N5)c3CO2)c1C(F)(F)F]

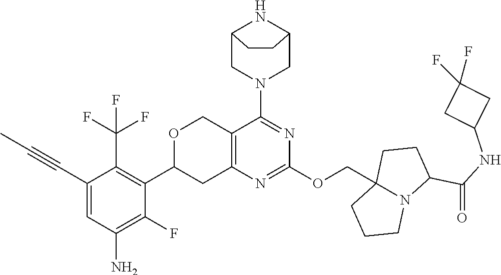 [SMILES: CC#Cc1cc(N)c(F)c(C2Cc3nc(OCC45CCCN4C(C(=O)NC4CC(F)(F)C4)CC5)nc(N4CC5CCC(C4)N5)c3CO2)c1C(F)(F)F]

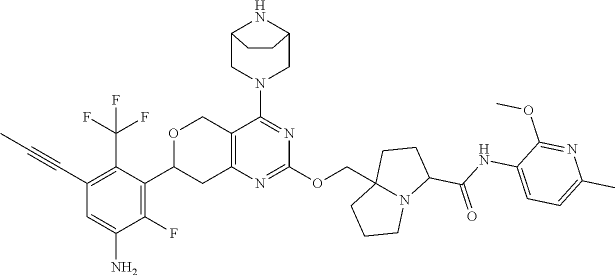 [SMILES: CC#Cc1cc(N)c(F)c(C2Cc3nc(OCC45CCCN4C(C(=O)Nc4ccc(C)nc4OC)CC5)nc(N4CC5CCC(C4)N5)c3CO2)c1C(F)(F)F]

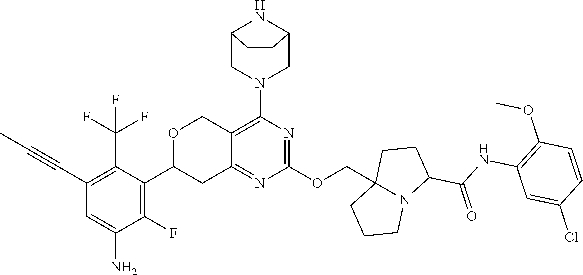 [SMILES: CC#Cc1cc(N)c(F)c(C2Cc3nc(OCC45CCCN4C(C(=O)Nc4cc(Cl)ccc4OC)CC5)nc(N4CC5CCC(C4)N5)c3CO2)c1C(F)(F)F]

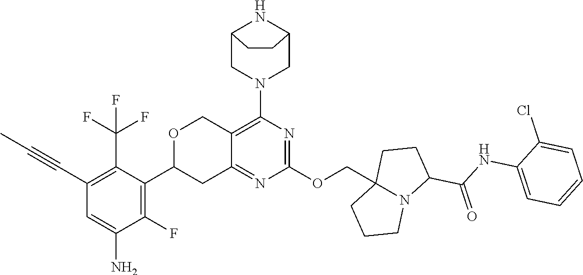 [SMILES: CC#Cc1cc(N)c(F)c(C2Cc3nc(OCC45CCCN4C(C(=O)Nc4ccccc4Cl)CC5)nc(N4CC5CCC(C4)N5)c3CO2)c1C(F)(F)F]

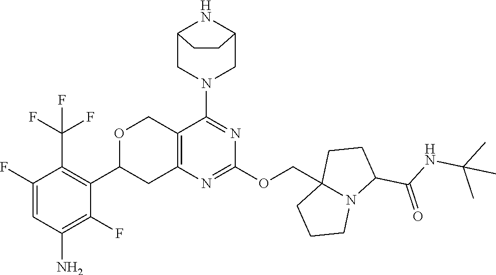 [SMILES: CC(C)(C)NC(=O)C1CCC2(COc3nc4c(c(N5CC6CCC(C5)N6)n3)COC(c3c(F)c(N)cc(F)c3C(F)(F)F)C4)CCCN12]

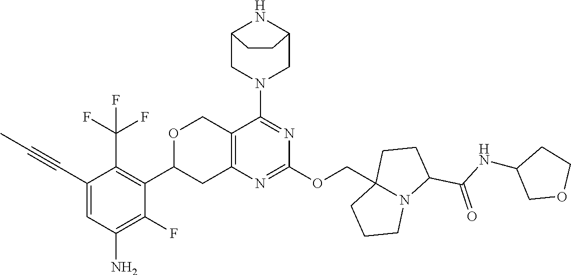 [SMILES: CC#Cc1cc(N)c(F)c(C2Cc3nc(OCC45CCCN4C(C(=O)NC4CCOC4)CC5)nc(N4CC5CCC(C4)N5)c3CO2)c1C(F)(F)F]

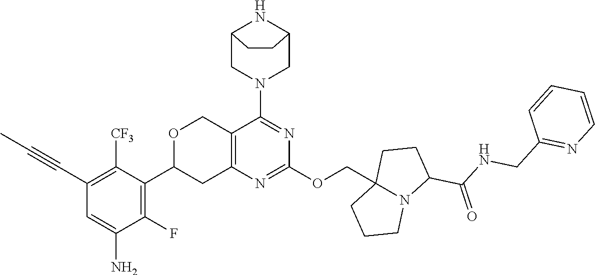 [SMILES: CC#Cc1cc(N)c(F)c(C2Cc3nc(OCC45CCCN4C(C(=O)NCc4ccccn4)CC5)nc(N4CC5CCC(C4)N5)c3CO2)c1C(F)(F)F]

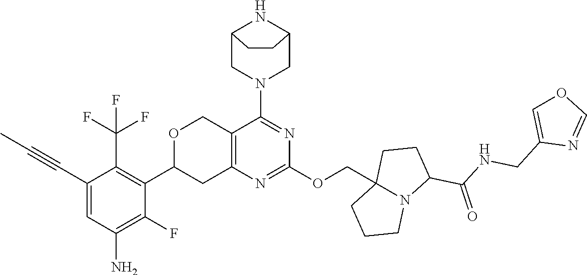 [SMILES: CC#Cc1cc(N)c(F)c(C2Cc3nc(OCC45CCCN4C(C(=O)NCc4cocn4)CC5)nc(N4CC5CCC(C4)N5)c3CO2)c1C(F)(F)F]

—

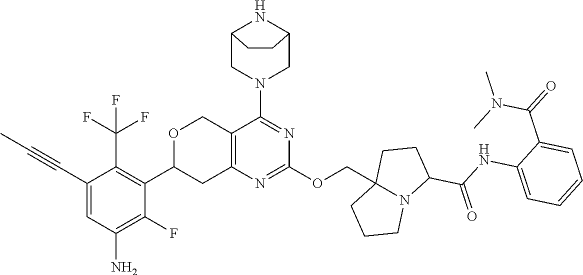 [SMILES: CC#Cc1cc(N)c(F)c(C2Cc3nc(OCC45CCCN4C(C(=O)Nc4ccccc4C(=O)N(C)C)CC5)nc(N4CC5CCC(C4)N5)c3CO2)c1C(F)(F)F]

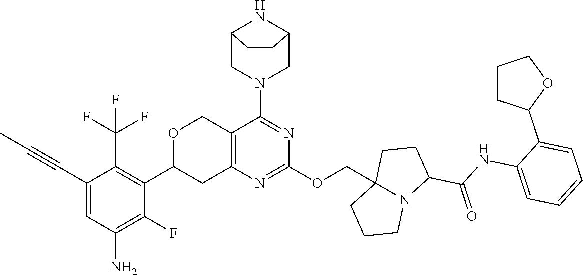 [SMILES: CC#Cc1cc(N)c(F)c(C2Cc3nc(OCC45CCCN4C(C(=O)Nc4ccccc4C4CCCO4)CC5)nc(N4CC5CCC(C4)N5)c3CO2)c1C(F)(F)F]

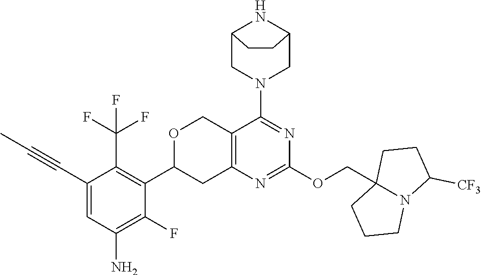 [SMILES: CC#Cc1cc(N)c(F)c(C2Cc3nc(OCC45CCCN4C(C(F)(F)F)CC5)nc(N4CC5CCC(C4)N5)c3CO2)c1C(F)(F)F]

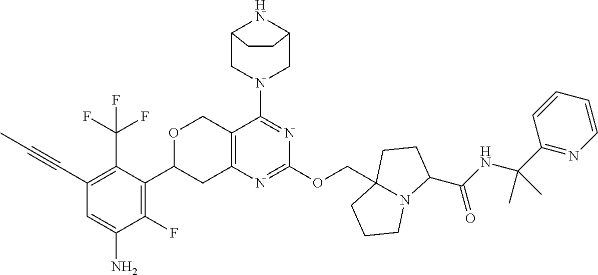 [SMILES: CC#Cc1cc(N)c(F)c(C2Cc3nc(OCC45CCCN4C(C(=O)NC(C)(C)c4ccccn4)CC5)nc(N4CC5CCC(C4)N5)c3CO2)c1C(F)(F)F]

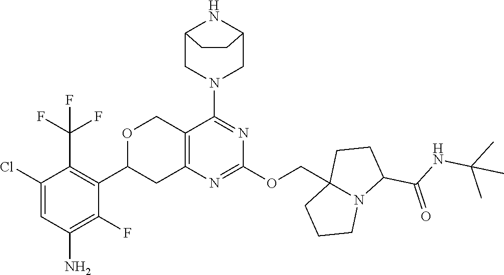 [SMILES: CC(C)(C)NC(=O)C1CCC2(COc3nc4c(c(N5CC6CCC(C5)N6)n3)COC(c3c(F)c(N)cc(Cl)c3C(F)(F)F)C4)CCCN12]

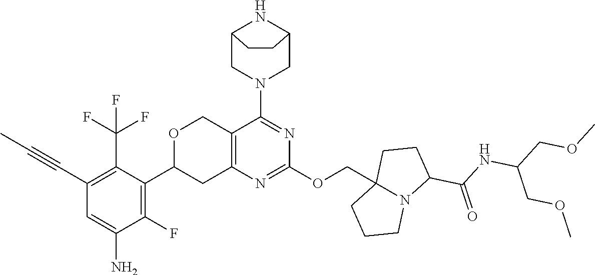 [SMILES: CC#Cc1cc(N)c(F)c(C2Cc3nc(OCC45CCCN4C(C(=O)NC(COC)COC)CC5)nc(N4CC5CCC(C4)N5)c3CO2)c1C(F)(F)F]

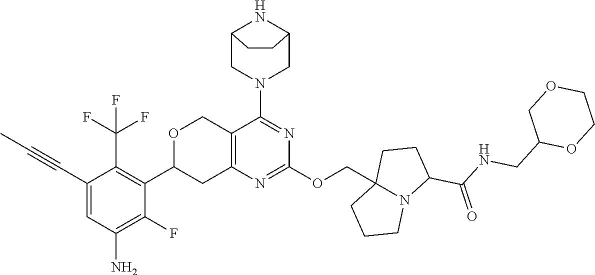 [SMILES: CC#Cc1cc(N)c(F)c(C2Cc3nc(OCC45CCCN4C(C(=O)NCC4COCCO4)CC5)nc(N4CC5CCC(C4)N5)c3CO2)c1C(F)(F)F]

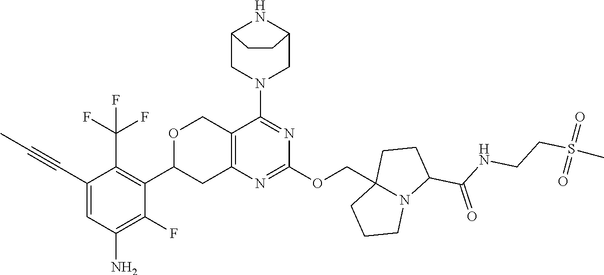 [SMILES: CC#Cc1cc(N)c(F)c(C2Cc3nc(OCC45CCCN4C(C(=O)NCCS(C)(=O)=O)CC5)nc(N4CC5CCC(C4)N5)c3CO2)c1C(F)(F)F]

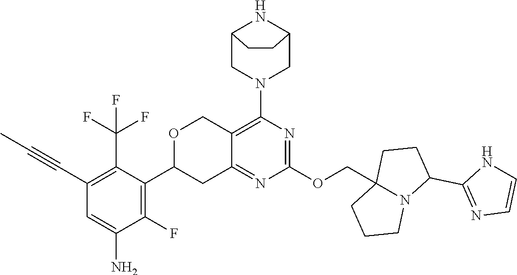 [SMILES: CC#Cc1cc(N)c(F)c(C2Cc3nc(OCC45CCCN4C(c4ncc[nH]4)CC5)nc(N4CC5CCC(C4)N5)c3CO2)c1C(F)(F)F]

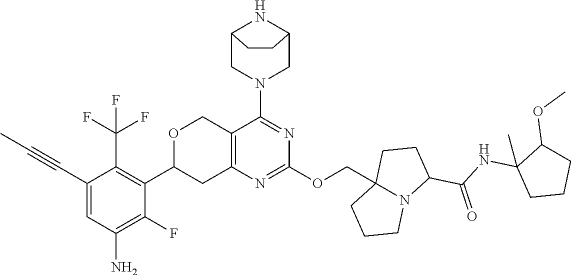 [SMILES: CC#Cc1cc(N)c(F)c(C2Cc3nc(OCC45CCCN4C(C(=O)NC4(C)CCCC4OC)CC5)nc(N4CC5CCC(C4)N5)c3CO2)c1C(F)(F)F]

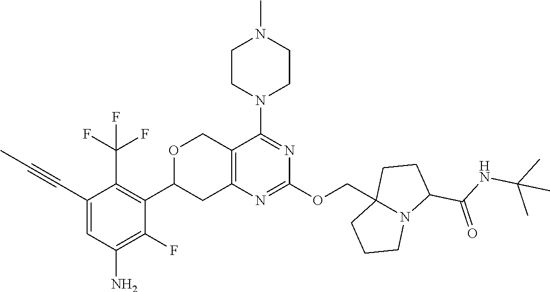 [SMILES: CC#Cc1cc(N)c(F)c(C2Cc3nc(OCC45CCCN4C(C(=O)NC(C)(C)C)CC5)nc(N4CCN(C)CC4)c3CO2)c1C(F)(F)F]

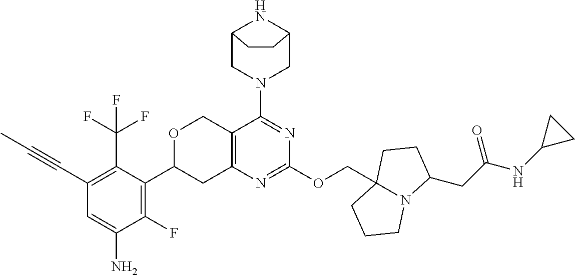 [SMILES: CC#Cc1cc(N)c(F)c(C2Cc3nc(OCC45CCCN4C(CC(=O)NC4CC4)CC5)nc(N4CC5CCC(C4)N5)c3CO2)c1C(F)(F)F]

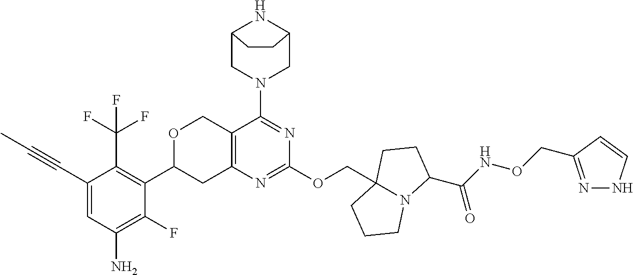 [SMILES: CC#Cc1cc(N)c(F)c(C2Cc3nc(OCC45CCCN4C(C(=O)NOCc4cc[nH]n4)CC5)nc(N4CC5CCC(C4)N5)c3CO2)c1C(F)(F)F]

—

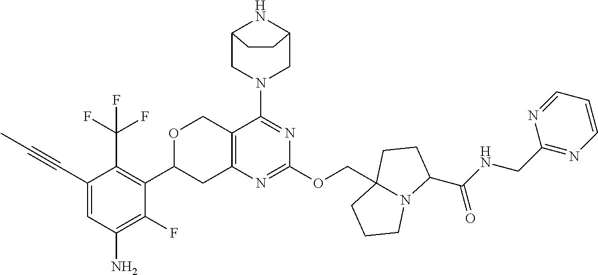 [SMILES: CC#Cc1cc(N)c(F)c(C2Cc3nc(OCC45CCCN4C(C(=O)NCc4ncccn4)CC5)nc(N4CC5CCC(C4)N5)c3CO2)c1C(F)(F)F]

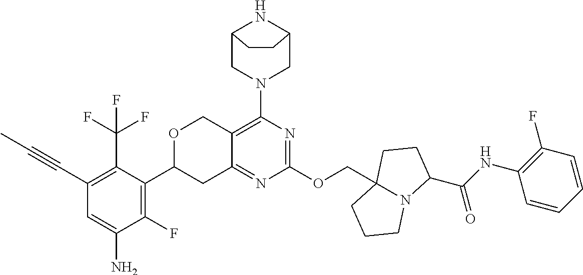 [SMILES: CC#Cc1cc(N)c(F)c(C2Cc3nc(OCC45CCCN4C(C(=O)Nc4ccccc4F)CC5)nc(N4CC5CCC(C4)N5)c3CO2)c1C(F)(F)F]

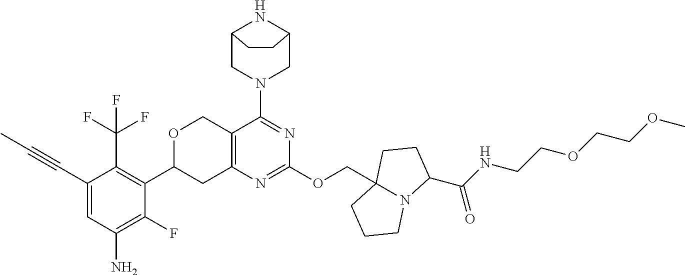 [SMILES: CC#Cc1cc(N)c(F)c(C2Cc3nc(OCC45CCCN4C(C(=O)NCCOCCOC)CC5)nc(N4CC5CCC(C4)N5)c3CO2)c1C(F)(F)F]

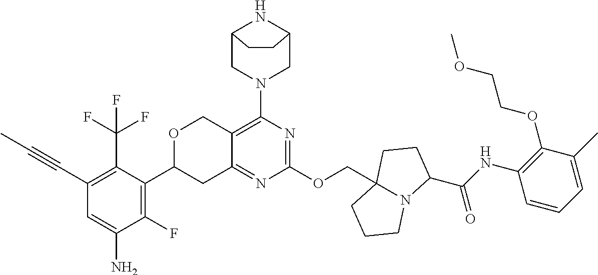 [SMILES: CC#Cc1cc(N)c(F)c(C2Cc3nc(OCC45CCCN4C(C(=O)Nc4cccc(C)c4OCCOC)CC5)nc(N4CC5CCC(C4)N5)c3CO2)c1C(F)(F)F]

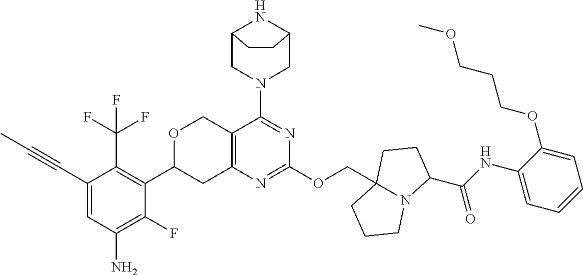 [SMILES: CC#Cc1cc(N)c(F)c(C2Cc3nc(OCC45CCCN4C(C(=O)Nc4ccccc4OCCCOC)CC5)nc(N4CC5CCC(C4)N5)c3CO2)c1C(F)(F)F]

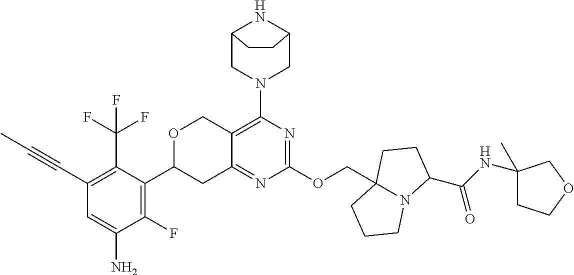 [SMILES: CC#Cc1cc(N)c(F)c(C2Cc3nc(OCC45CCCN4C(C(=O)NC4(C)CCOC4)CC5)nc(N4CC5CCC(C4)N5)c3CO2)c1C(F)(F)F]

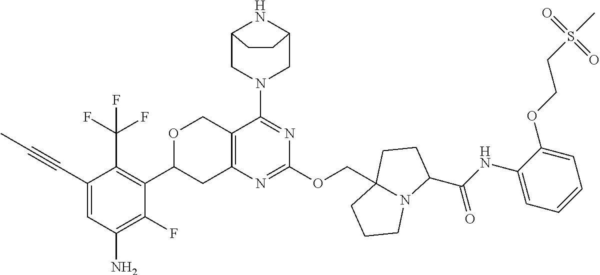 [SMILES: CC#Cc1cc(N)c(F)c(C2Cc3nc(OCC45CCCN4C(C(=O)Nc4ccccc4OCCS(C)(=O)=O)CC5)nc(N4CC5CCC(C4)N5)c3CO2)c1C(F)(F)F]

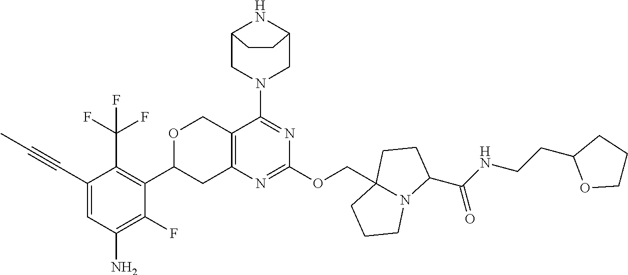 [SMILES: CC#Cc1cc(N)c(F)c(C2Cc3nc(OCC45CCCN4C(C(=O)NCCC4CCCO4)CC5)nc(N4CC5CCC(C4)N5)c3CO2)c1C(F)(F)F]

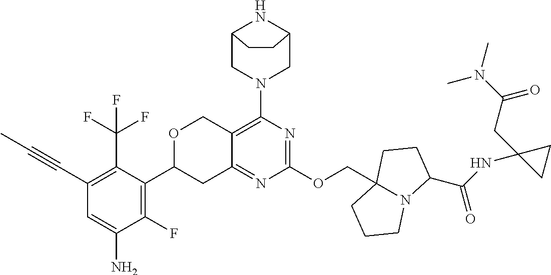 [SMILES: CC#Cc1cc(N)c(F)c(C2Cc3nc(OCC45CCCN4C(C(=O)NC4(CC(=O)N(C)C)CC4)CC5)nc(N4CC5CCC(C4)N5)c3CO2)c1C(F)(F)F]

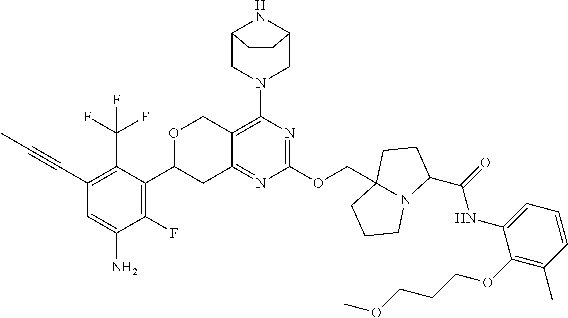 [SMILES: CC#Cc1cc(N)c(F)c(C2Cc3nc(OCC45CCCN4C(C(=O)Nc4cccc(C)c4OCCCOC)CC5)nc(N4CC5CCC(C4)N5)c3CO2)c1C(F)(F)F]

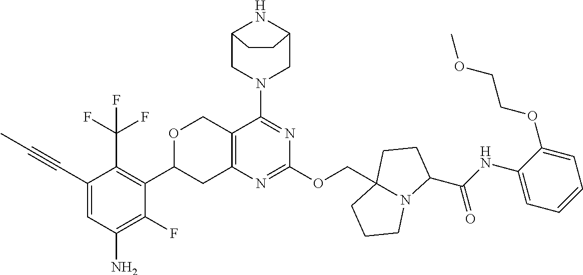 [SMILES: CC#Cc1cc(N)c(F)c(C2Cc3nc(OCC45CCCN4C(C(=O)Nc4ccccc4OCCOC)CC5)nc(N4CC5CCC(C4)N5)c3CO2)c1C(F)(F)F]

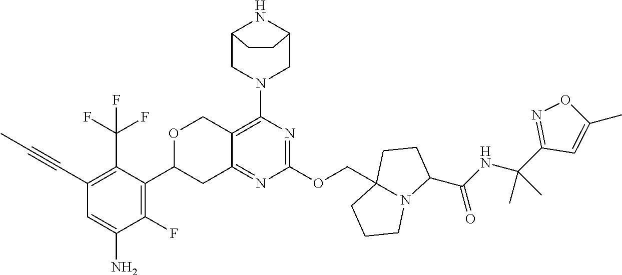 [SMILES: CC#Cc1cc(N)c(F)c(C2Cc3nc(OCC45CCCN4C(C(=O)NC(C)(C)c4cc(C)on4)CC5)nc(N4CC5CCC(C4)N5)c3CO2)c1C(F)(F)F]

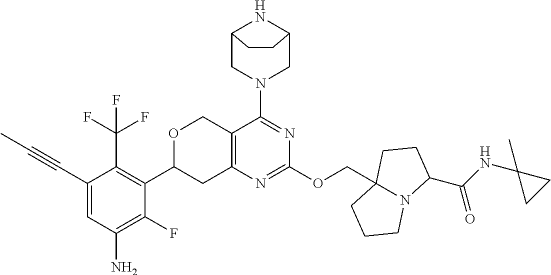 [SMILES: CC#Cc1cc(N)c(F)c(C2Cc3nc(OCC45CCCN4C(C(=O)NC4(C)CC4)CC5)nc(N4CC5CCC(C4)N5)c3CO2)c1C(F)(F)F]

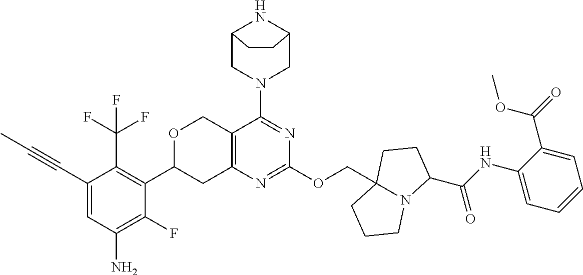 [SMILES: CC#Cc1cc(N)c(F)c(C2Cc3nc(OCC45CCCN4C(C(=O)Nc4ccccc4C(=O)OC)CC5)nc(N4CC5CCC(C4)N5)c3CO2)c1C(F)(F)F]

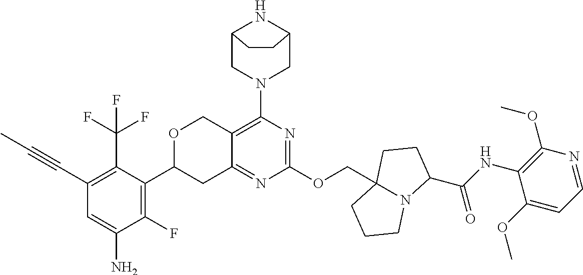 [SMILES: CC#Cc1cc(N)c(F)c(C2Cc3nc(OCC45CCCN4C(C(=O)Nc4c(OC)ccnc4OC)CC5)nc(N4CC5CCC(C4)N5)c3CO2)c1C(F)(F)F]

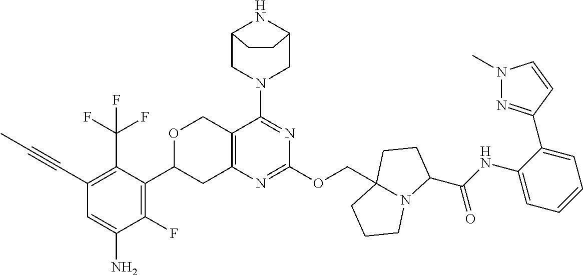 [SMILES: CC#Cc1cc(N)c(F)c(C2Cc3nc(OCC45CCCN4C(C(=O)Nc4ccccc4-c4ccn(C)n4)CC5)nc(N4CC5CCC(C4)N5)c3CO2)c1C(F)(F)F]

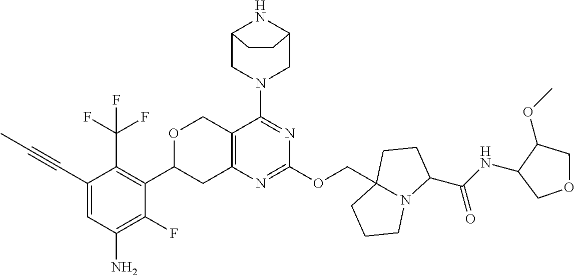 [SMILES: CC#Cc1cc(N)c(F)c(C2Cc3nc(OCC45CCCN4C(C(=O)NC4COCC4OC)CC5)nc(N4CC5CCC(C4)N5)c3CO2)c1C(F)(F)F]

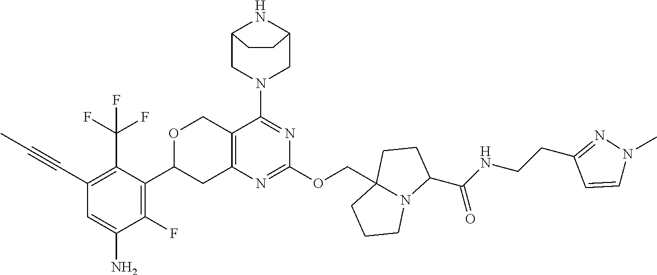 [SMILES: CC#Cc1cc(N)c(F)c(C2Cc3nc(OCC45CCCN4C(C(=O)NCCc4ccn(C)n4)CC5)nc(N4CC5CCC(C4)N5)c3CO2)c1C(F)(F)F]

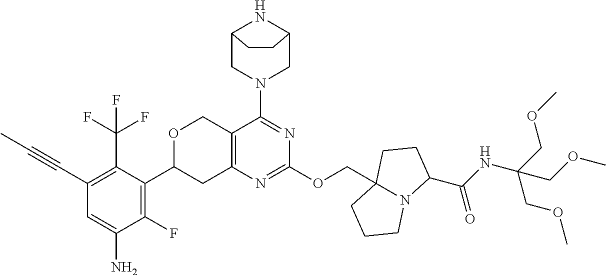 [SMILES: CC#Cc1cc(N)c(F)c(C2Cc3nc(OCC45CCCN4C(C(=O)NC(COC)(COC)COC)CC5)nc(N4CC5CCC(C4)N5)c3CO2)c1C(F)(F)F]

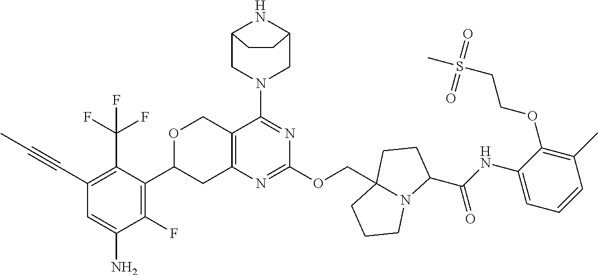 [SMILES: CC#Cc1cc(N)c(F)c(C2Cc3nc(OCC45CCCN4C(C(=O)Nc4cccc(C)c4OCCS(C)(=O)=O)CC5)nc(N4CC5CCC(C4)N5)c3CO2)c1C(F)(F)F]

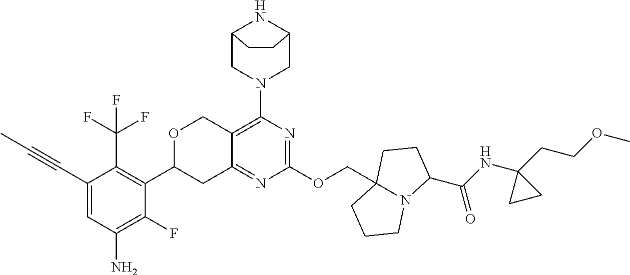 [SMILES: CC#Cc1cc(N)c(F)c(C2Cc3nc(OCC45CCCN4C(C(=O)NC4(CCOC)CC4)CC5)nc(N4CC5CCC(C4)N5)c3CO2)c1C(F)(F)F]

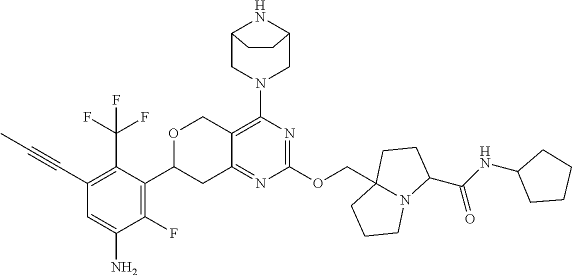 [SMILES: CC#Cc1cc(N)c(F)c(C2Cc3nc(OCC45CCCN4C(C(=O)NC4CCCC4)CC5)nc(N4CC5CCC(C4)N5)c3CO2)c1C(F)(F)F]

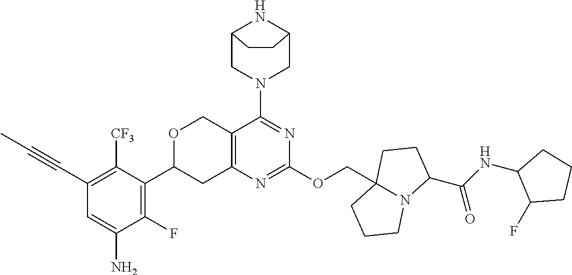 [SMILES: CC#Cc1cc(N)c(F)c(C2Cc3nc(OCC45CCCN4C(C(=O)NC4CCCC4F)CC5)nc(N4CC5CCC(C4)N5)c3CO2)c1C(F)(F)F]

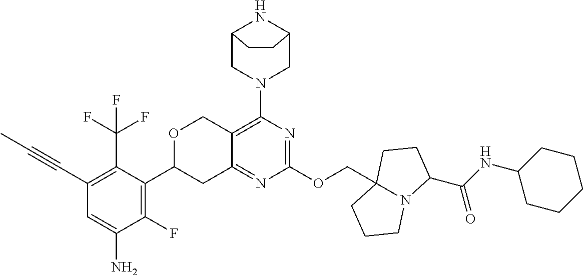 [SMILES: CC#Cc1cc(N)c(F)c(C2Cc3nc(OCC45CCCN4C(C(=O)NC4CCCCC4)CC5)nc(N4CC5CCC(C4)N5)c3CO2)c1C(F)(F)F]

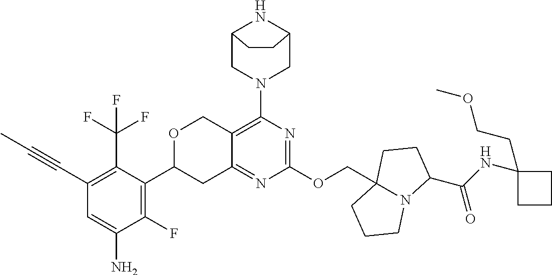 [SMILES: CC#Cc1cc(N)c(F)c(C2Cc3nc(OCC45CCCN4C(C(=O)NC4(CCOC)CCC4)CC5)nc(N4CC5CCC(C4)N5)c3CO2)c1C(F)(F)F]

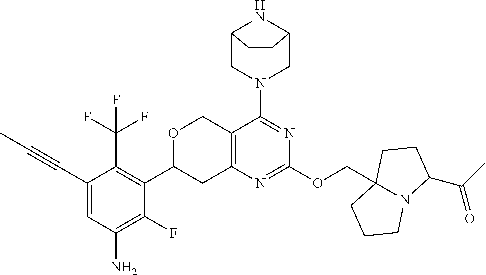 [SMILES: CC#Cc1cc(N)c(F)c(C2Cc3nc(OCC45CCCN4C(C(C)=O)CC5)nc(N4CC5CCC(C4)N5)c3CO2)c1C(F)(F)F]

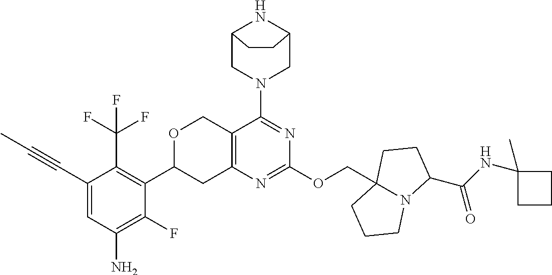 [SMILES: CC#Cc1cc(N)c(F)c(C2Cc3nc(OCC45CCCN4C(C(=O)NC4(C)CCC4)CC5)nc(N4CC5CCC(C4)N5)c3CO2)c1C(F)(F)F]

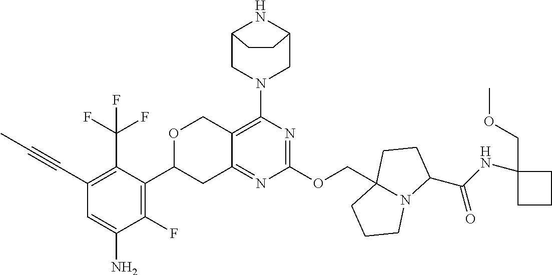 [SMILES: CC#Cc1cc(N)c(F)c(C2Cc3nc(OCC45CCCN4C(C(=O)NC4(COC)CCC4)CC5)nc(N4CC5CCC(C4)N5)c3CO2)c1C(F)(F)F]

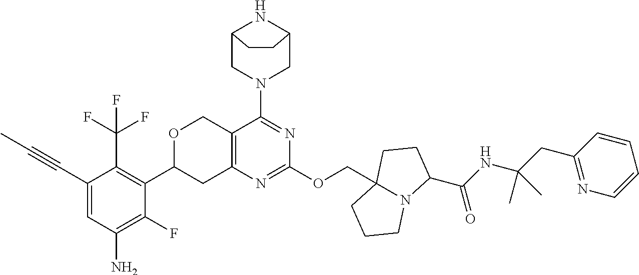 [SMILES: CC#Cc1cc(N)c(F)c(C2Cc3nc(OCC45CCCN4C(C(=O)NC(C)(C)Cc4ccccn4)CC5)nc(N4CC5CCC(C4)N5)c3CO2)c1C(F)(F)F]

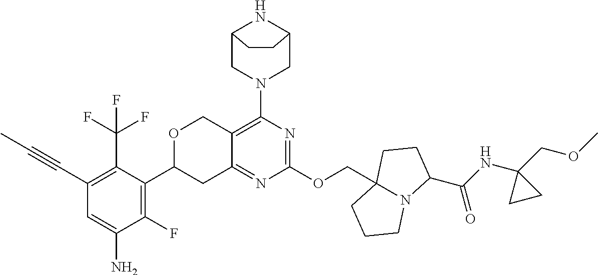 [SMILES: CC#Cc1cc(N)c(F)c(C2Cc3nc(OCC45CCCN4C(C(=O)NC4(COC)CC4)CC5)nc(N4CC5CCC(C4)N5)c3CO2)c1C(F)(F)F]

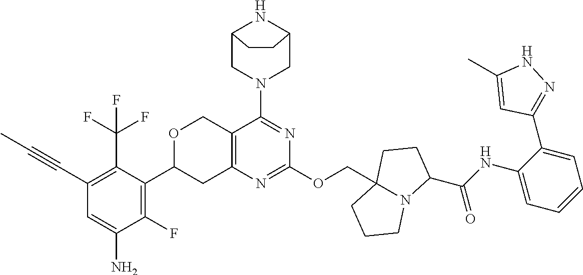 [SMILES: CC#Cc1cc(N)c(F)c(C2Cc3nc(OCC45CCCN4C(C(=O)Nc4ccccc4-c4cc(C)[nH]n4)CC5)nc(N4CC5CCC(C4)N5)c3CO2)c1C(F)(F)F]

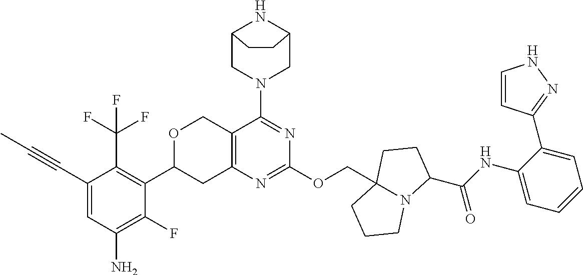 [SMILES: CC#Cc1cc(N)c(F)c(C2Cc3nc(OCC45CCCN4C(C(=O)Nc4ccccc4-c4cc[nH]n4)CC5)nc(N4CC5CCC(C4)N5)c3CO2)c1C(F)(F)F]

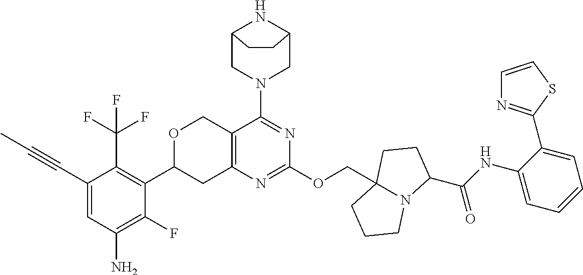 [SMILES: CC#Cc1cc(N)c(F)c(C2Cc3nc(OCC45CCCN4C(C(=O)Nc4ccccc4-c4nccs4)CC5)nc(N4CC5CCC(C4)N5)c3CO2)c1C(F)(F)F]

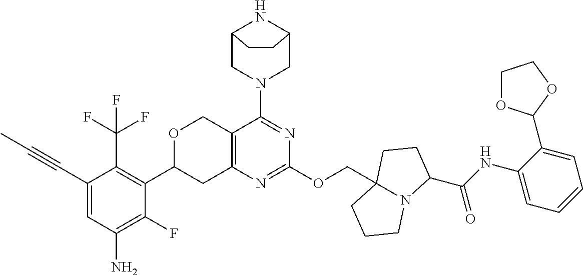 [SMILES: CC#Cc1cc(N)c(F)c(C2Cc3nc(OCC45CCCN4C(C(=O)Nc4ccccc4C4OCCO4)CC5)nc(N4CC5CCC(C4)N5)c3CO2)c1C(F)(F)F]

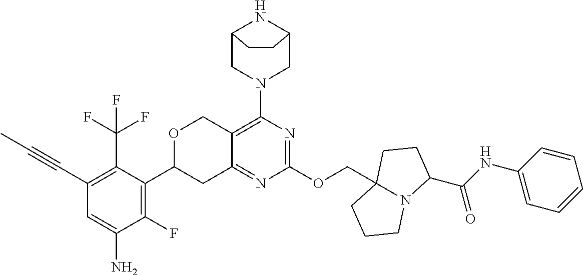 [SMILES: CC#Cc1cc(N)c(F)c(C2Cc3nc(OCC45CCCN4C(C(=O)Nc4ccccc4)CC5)nc(N4CC5CCC(C4)N5)c3CO2)c1C(F)(F)F]

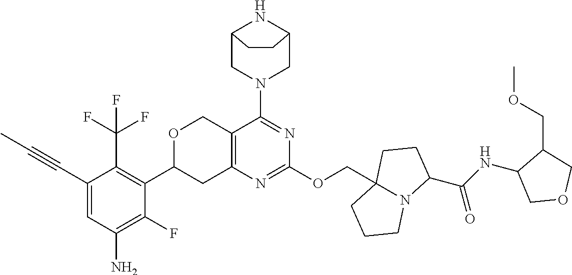 [SMILES: CC#Cc1cc(N)c(F)c(C2Cc3nc(OCC45CCCN4C(C(=O)NC4COCC4COC)CC5)nc(N4CC5CCC(C4)N5)c3CO2)c1C(F)(F)F]

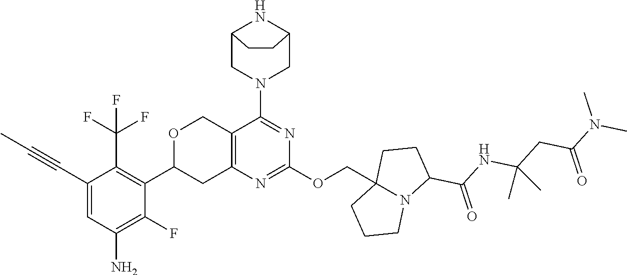 [SMILES: CC#Cc1cc(N)c(F)c(C2Cc3nc(OCC45CCCN4C(C(=O)NC(C)(C)CC(=O)N(C)C)CC5)nc(N4CC5CCC(C4)N5)c3CO2)c1C(F)(F)F]

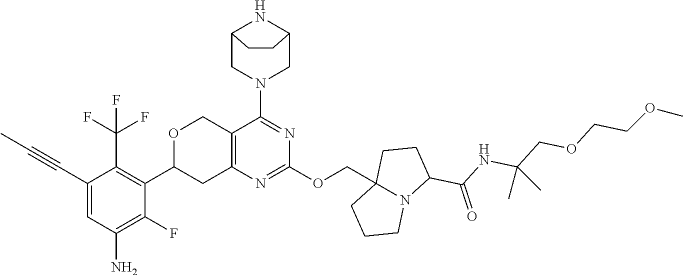 [SMILES: CC#Cc1cc(N)c(F)c(C2Cc3nc(OCC45CCCN4C(C(=O)NC(C)(C)COCCOC)CC5)nc(N4CC5CCC(C4)N5)c3CO2)c1C(F)(F)F]

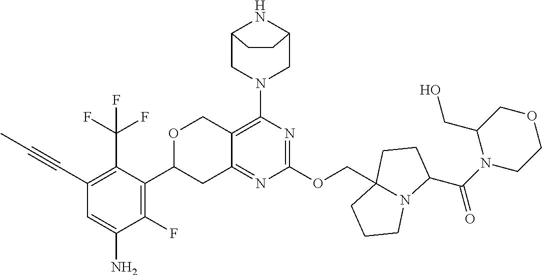 [SMILES: CC#Cc1cc(N)c(F)c(C2Cc3nc(OCC45CCCN4C(C(=O)N4CCOCC4CO)CC5)nc(N4CC5CCC(C4)N5)c3CO2)c1C(F)(F)F]

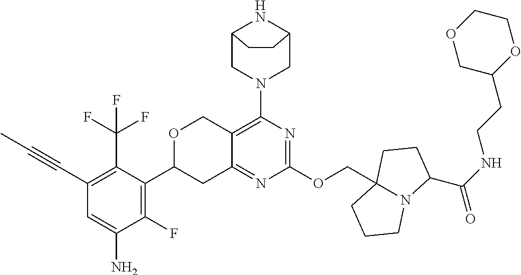 [SMILES: CC#Cc1cc(N)c(F)c(C2Cc3nc(OCC45CCCN4C(C(=O)NCCC4COCCO4)CC5)nc(N4CC5CCC(C4)N5)c3CO2)c1C(F)(F)F]

TABLE 2

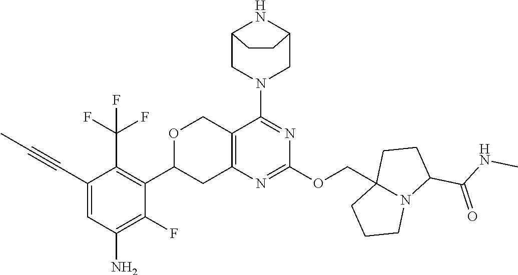 [SMILES: CC#Cc1cc(N)c(F)c(C2Cc3nc(OCC45CCCN4C(C(=O)NC)CC5)nc(N4CC5CCC(C4)N5)c3CO2)c1C(F)(F)F]

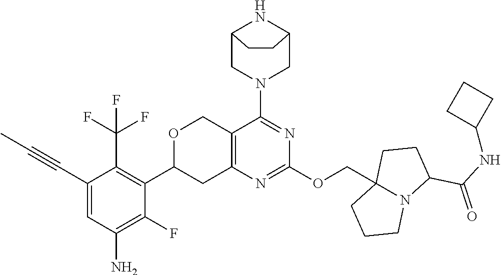 [SMILES: CC#Cc1cc(N)c(F)c(C2Cc3nc(OCC45CCCN4C(C(=O)NC4CCC4)CC5)nc(N4CC5CCC(C4)N5)c3CO2)c1C(F)(F)F]

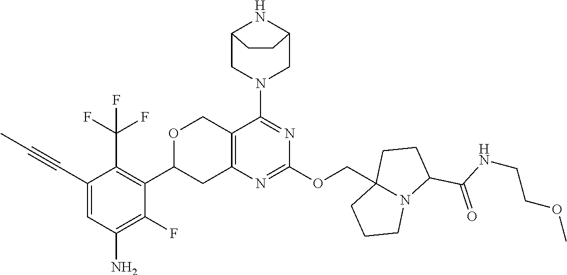 [SMILES: CC#Cc1cc(N)c(F)c(C2Cc3nc(OCC45CCCN4C(C(=O)NCCOC)CC5)nc(N4CC5CCC(C4)N5)c3CO2)c1C(F)(F)F]

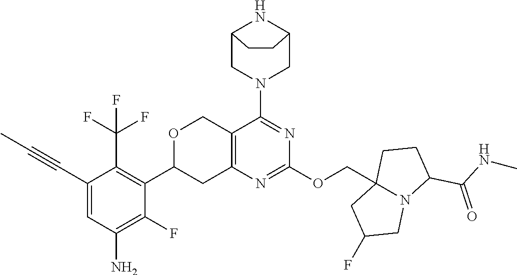 [SMILES: CC#Cc1cc(N)c(F)c(C2Cc3nc(OCC45CCC(C(=O)NC)N4CC(F)C5)nc(N4CC5CCC(C4)N5)c3CO2)c1C(F)(F)F]

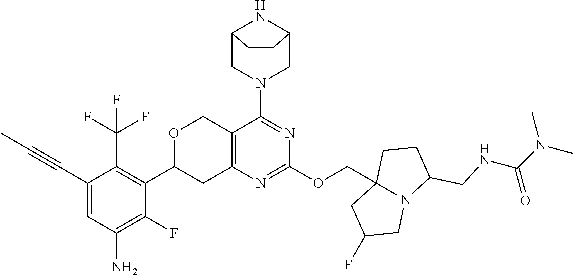 [SMILES: CC#Cc1cc(N)c(F)c(C2Cc3nc(OCC45CCC(CNC(=O)N(C)C)N4CC(F)C5)nc(N4CC5CCC(C4)N5)c3CO2)c1C(F)(F)F]

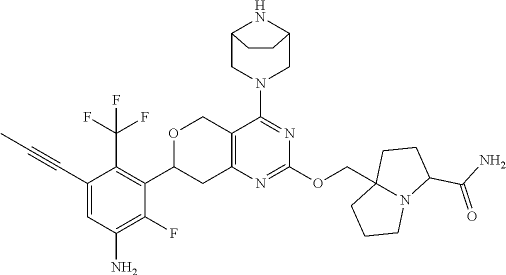 [SMILES: CC#Cc1cc(N)c(F)c(C2Cc3nc(OCC45CCCN4C(C(N)=O)CC5)nc(N4CC5CCC(C4)N5)c3CO2)c1C(F)(F)F]

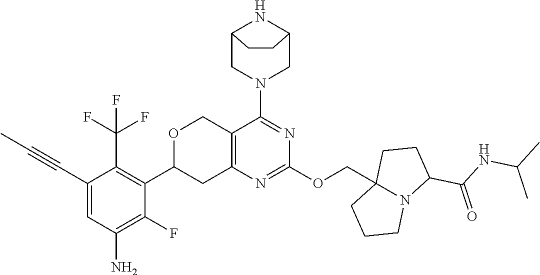 [SMILES: CC#Cc1cc(N)c(F)c(C2Cc3nc(OCC45CCCN4C(C(=O)NC(C)C)CC5)nc(N4CC5CCC(C4)N5)c3CO2)c1C(F)(F)F]

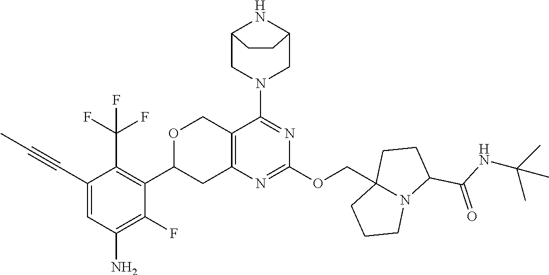 [SMILES: CC#Cc1cc(N)c(F)c(C2Cc3nc(OCC45CCCN4C(C(=O)NC(C)(C)C)CC5)nc(N4CC5CCC(C4)N5)c3CO2)c1C(F)(F)F]

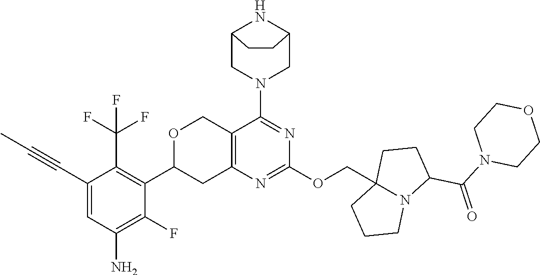 [SMILES: CC#Cc1cc(N)c(F)c(C2Cc3nc(OCC45CCCN4C(C(=O)N4CCOCC4)CC5)nc(N4CC5CCC(C4)N5)c3CO2)c1C(F)(F)F]

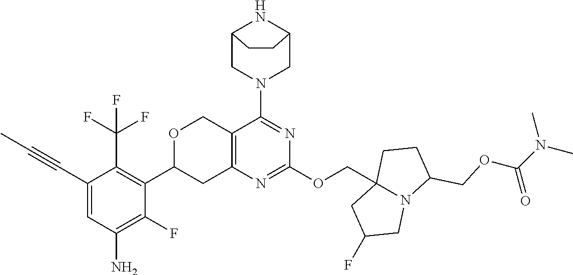 [SMILES: CC#Cc1cc(N)c(F)c(C2Cc3nc(OCC45CCC(COC(=O)N(C)C)N4CC(F)C5)nc(N4CC5CCC(C4)N5)c3CO2)c1C(F)(F)F]

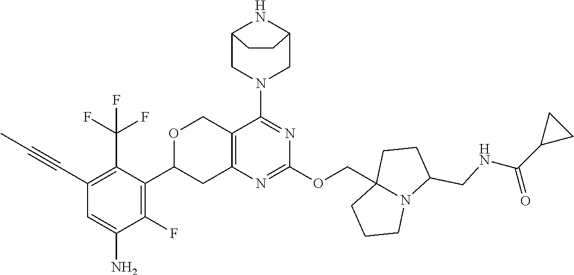 [SMILES: CC#Cc1cc(N)c(F)c(C2Cc3nc(OCC45CCCN4C(CNC(=O)C4CC4)CC5)nc(N4CC5CCC(C4)N5)c3CO2)c1C(F)(F)F]

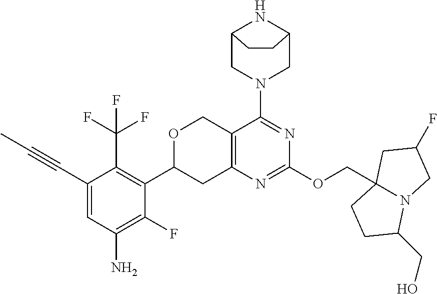 [SMILES: CC#Cc1cc(N)c(F)c(C2Cc3nc(OCC45CCC(CO)N4CC(F)C5)nc(N4CC5CCC(C4)N5)c3CO2)c1C(F)(F)F]

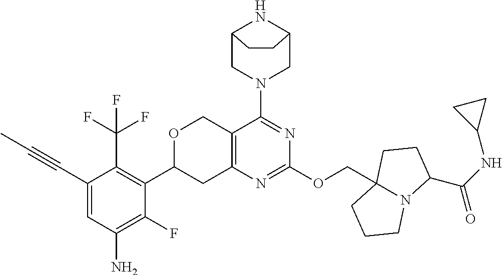 [SMILES: CC#Cc1cc(N)c(F)c(C2Cc3nc(OCC45CCCN4C(C(=O)NC4CC4)CC5)nc(N4CC5CCC(C4)N5)c3CO2)c1C(F)(F)F]

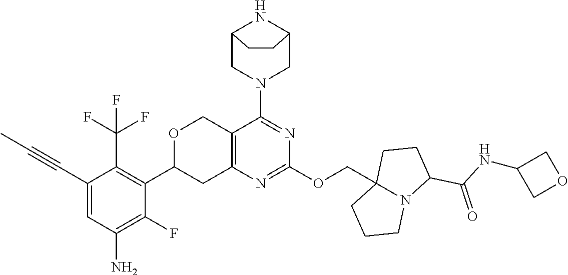 [SMILES: CC#Cc1cc(N)c(F)c(C2Cc3nc(OCC45CCCN4C(C(=O)NC4COC4)CC5)nc(N4CC5CCC(C4)N5)c3CO2)c1C(F)(F)F]

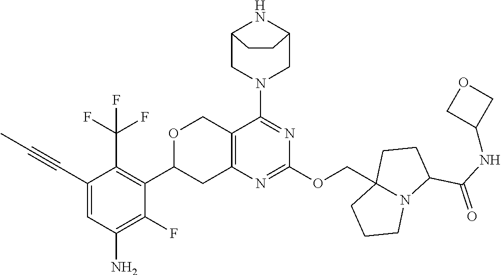 [SMILES: CC#Cc1cc(N)c(F)c(C2Cc3nc(OCC45CCCN4C(C(=O)NC4COC4)CC5)nc(N4CC5CCC(C4)N5)c3CO2)c1C(F)(F)F]

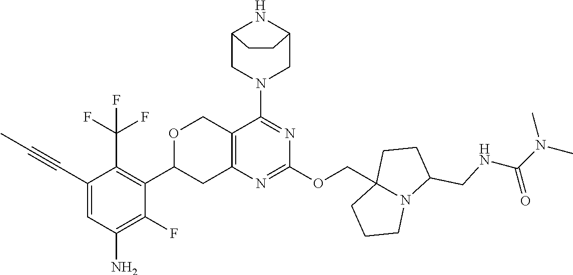 [SMILES: CC#Cc1cc(N)c(F)c(C2Cc3nc(OCC45CCCN4C(CNC(=O)N(C)C)CC5)nc(N4CC5CCC(C4)N5)c3CO2)c1C(F)(F)F]

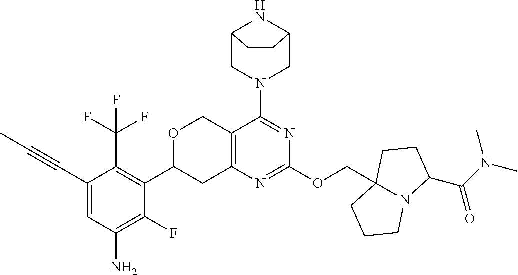 [SMILES: CC#Cc1cc(N)c(F)c(C2Cc3nc(OCC45CCCN4C(C(=O)N(C)C)CC5)nc(N4CC5CCC(C4)N5)c3CO2)c1C(F)(F)F]

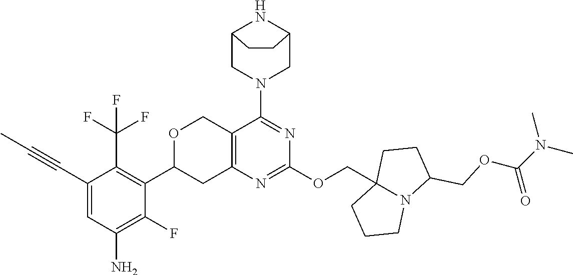 [SMILES: CC#Cc1cc(N)c(F)c(C2Cc3nc(OCC45CCCN4C(COC(=O)N(C)C)CC5)nc(N4CC5CCC(C4)N5)c3CO2)c1C(F)(F)F]

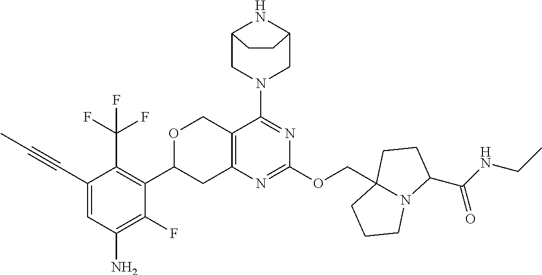 [SMILES: CC#Cc1cc(N)c(F)c(C2Cc3nc(OCC45CCCN4C(C(=O)NCC)CC5)nc(N4CC5CCC(C4)N5)c3CO2)c1C(F)(F)F]

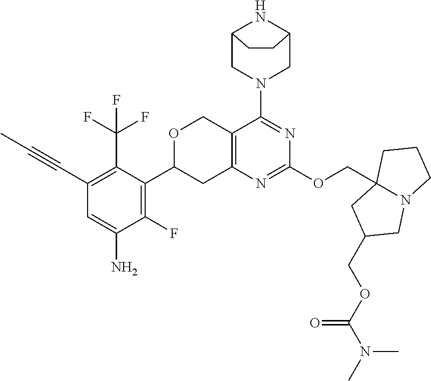 [SMILES: CC#Cc1cc(N)c(F)c(C2Cc3nc(OCC45CCCN4CC(COC(=O)N(C)C)C5)nc(N4CC5CCC(C4)N5)c3CO2)c1C(F)(F)F]

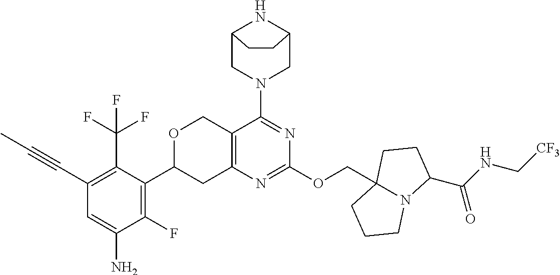 [SMILES: CC#Cc1cc(N)c(F)c(C2Cc3nc(OCC45CCCN4C(C(=O)NCC(F)(F)F)CC5)nc(N4CC5CCC(C4)N5)c3CO2)c1C(F)(F)F]

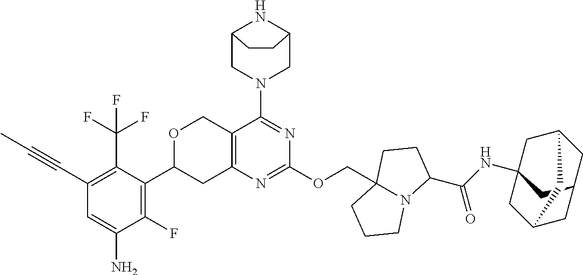 [SMILES: CC#Cc1cc(N)c(F)c(C2Cc3nc(OCC45CCCN4C(C(=O)N[C@]46C[C@H]7C[C@H](C[C@H](C7)C4)C6)CC5)nc(N4CC5CCC(C4)N5)c3CO2)c1C(F)(F)F]

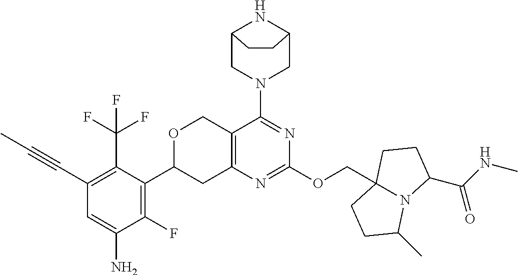 [SMILES: CC#Cc1cc(N)c(F)c(C2Cc3nc(OCC45CCC(C)N4C(C(=O)NC)CC5)nc(N4CC5CCC(C4)N5)c3CO2)c1C(F)(F)F]

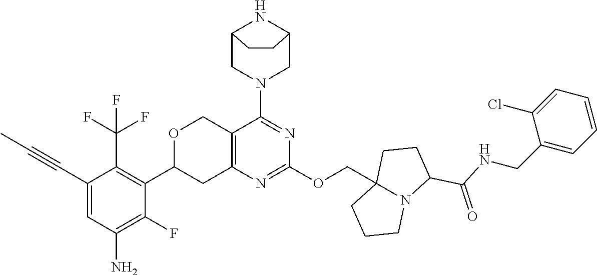 [SMILES: CC#Cc1cc(N)c(F)c(C2Cc3nc(OCC45CCCN4C(C(=O)NCc4ccccc4Cl)CC5)nc(N4CC5CCC(C4)N5)c3CO2)c1C(F)(F)F]

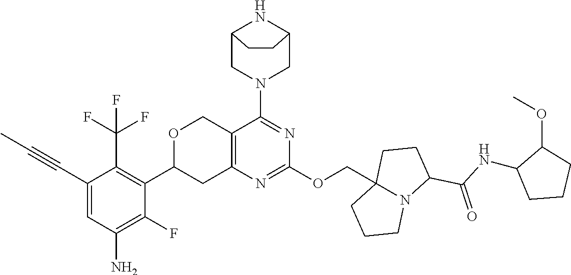 [SMILES: CC#Cc1cc(N)c(F)c(C2Cc3nc(OCC45CCCN4C(C(=O)NC4CCCC4OC)CC5)nc(N4CC5CCC(C4)N5)c3CO2)c1C(F)(F)F]

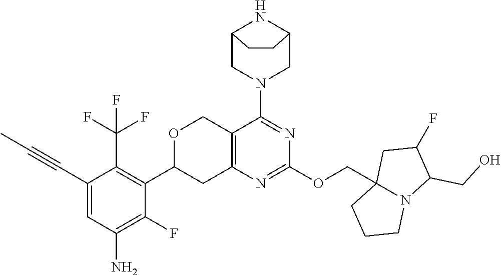 [SMILES: CC#Cc1cc(N)c(F)c(C2Cc3nc(OCC45CCCN4C(CO)C(F)C5)nc(N4CC5CCC(C4)N5)c3CO2)c1C(F)(F)F]

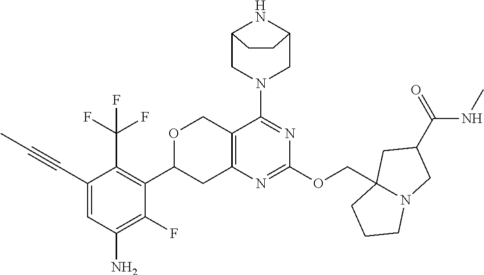 [SMILES: CC#Cc1cc(N)c(F)c(C2Cc3nc(OCC45CCCN4CC(C(=O)NC)C5)nc(N4CC5CCC(C4)N5)c3CO2)c1C(F)(F)F]

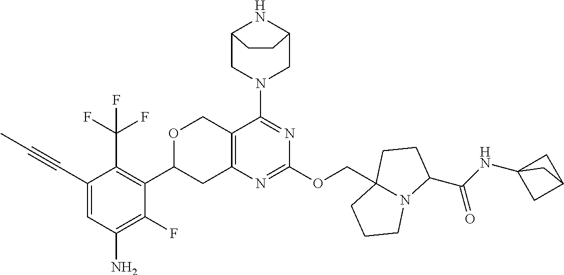 [SMILES: CC#Cc1cc(N)c(F)c(C2Cc3nc(OCC45CCCN4C(C(=O)NC46CC(C4)C6)CC5)nc(N4CC5CCC(C4)N5)c3CO2)c1C(F)(F)F]

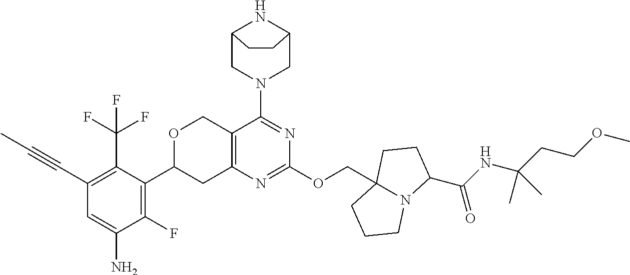 [SMILES: CC#Cc1cc(N)c(F)c(C2Cc3nc(OCC45CCCN4C(C(=O)NC(C)(C)CCOC)CC5)nc(N4CC5CCC(C4)N5)c3CO2)c1C(F)(F)F]

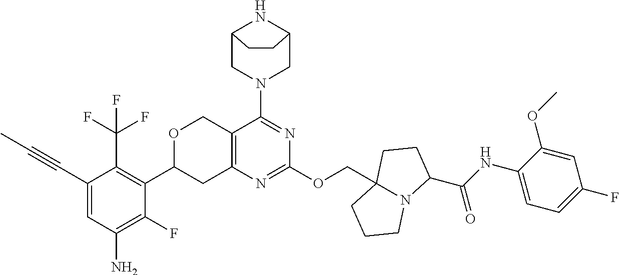 [SMILES: CC#Cc1cc(N)c(F)c(C2Cc3nc(OCC45CCCN4C(C(=O)Nc4ccc(F)cc4OC)CC5)nc(N4CC5CCC(C4)N5)c3CO2)c1C(F)(F)F]

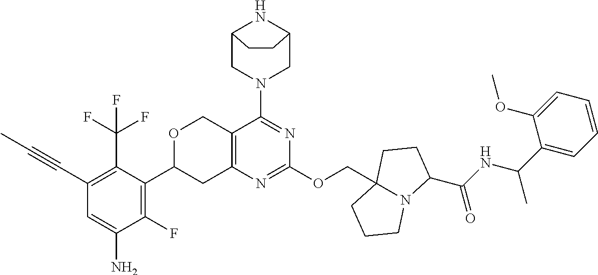 [SMILES: CC#Cc1cc(N)c(F)c(C2Cc3nc(OCC45CCCN4C(C(=O)NC(C)c4ccccc4OC)CC5)nc(N4CC5CCC(C4)N5)c3CO2)c1C(F)(F)F]

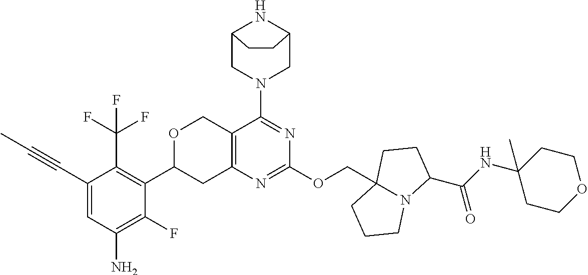 [SMILES: CC#Cc1cc(N)c(F)c(C2Cc3nc(OCC45CCCN4C(C(=O)NC4(C)CCOCC4)CC5)nc(N4CC5CCC(C4)N5)c3CO2)c1C(F)(F)F]

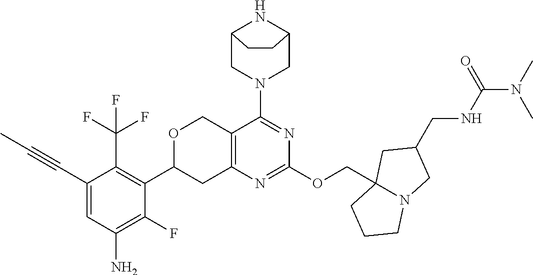 [SMILES: CC#Cc1cc(N)c(F)c(C2Cc3nc(OCC45CCCN4CC(CNC(=O)N(C)C)C5)nc(N4CC5CCC(C4)N5)c3CO2)c1C(F)(F)F]

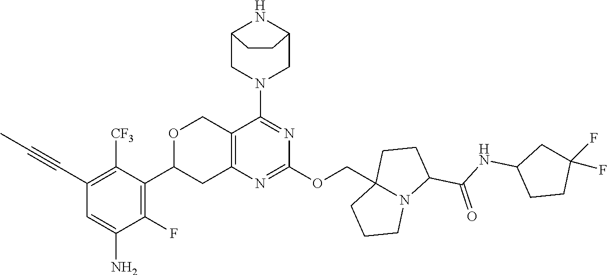 [SMILES: CC#Cc1cc(N)c(F)c(C2Cc3nc(OCC45CCCN4C(C(=O)NC4CCC(F)(F)C4)CC5)nc(N4CC5CCC(C4)N5)c3CO2)c1C(F)(F)F]

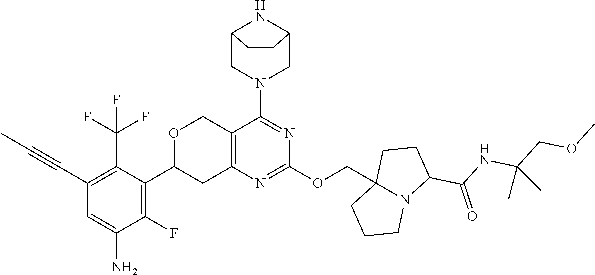 [SMILES: CC#Cc1cc(N)c(F)c(C2Cc3nc(OCC45CCCN4C(C(=O)NC(C)(C)COC)CC5)nc(N4CC5CCC(C4)N5)c3CO2)c1C(F)(F)F]

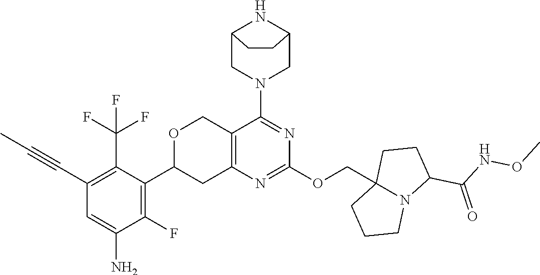 [SMILES: CC#Cc1cc(N)c(F)c(C2Cc3nc(OCC45CCCN4C(C(=O)NOC)CC5)nc(N4CC5CCC(C4)N5)c3CO2)c1C(F)(F)F]

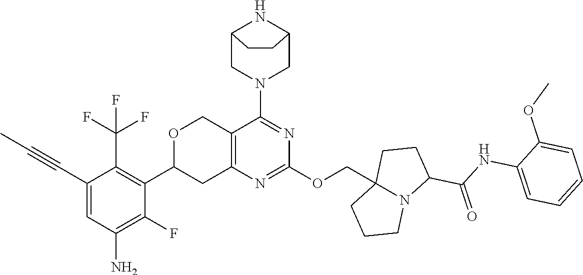 [SMILES: CC#Cc1cc(N)c(F)c(C2Cc3nc(OCC45CCCN4C(C(=O)Nc4ccccc4OC)CC5)nc(N4CC5CCC(C4)N5)c3CO2)c1C(F)(F)F]

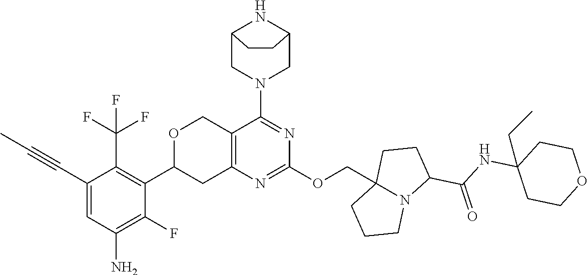 [SMILES: CC#Cc1cc(N)c(F)c(C2Cc3nc(OCC45CCCN4C(C(=O)NC4(CC)CCOCC4)CC5)nc(N4CC5CCC(C4)N5)c3CO2)c1C(F)(F)F]

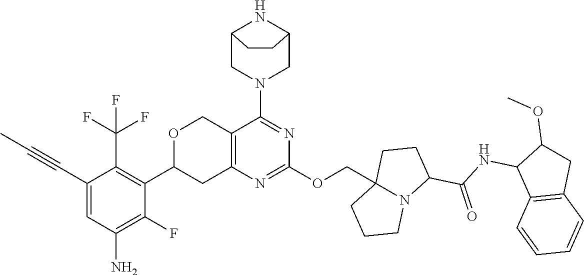 [SMILES: CC#Cc1cc(N)c(F)c(C2Cc3nc(OCC45CCCN4C(C(=O)NC4c6ccccc6CC4OC)CC5)nc(N4CC5CCC(C4)N5)c3CO2)c1C(F)(F)F]

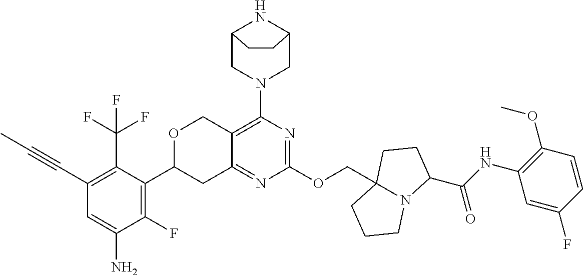 [SMILES: CC#Cc1cc(N)c(F)c(C2Cc3nc(OCC45CCCN4C(C(=O)Nc4cc(F)ccc4OC)CC5)nc(N4CC5CCC(C4)N5)c3CO2)c1C(F)(F)F]

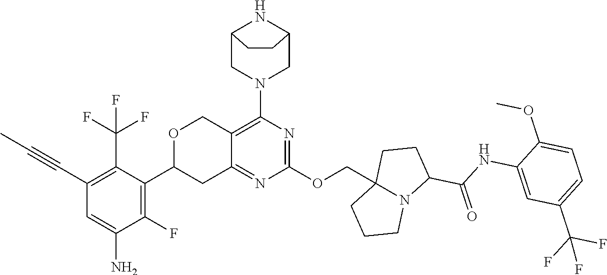 [SMILES: CC#Cc1cc(N)c(F)c(C2Cc3nc(OCC45CCCN4C(C(=O)Nc4cc(C(F)(F)F)ccc4OC)CC5)nc(N4CC5CCC(C4)N5)c3CO2)c1C(F)(F)F]

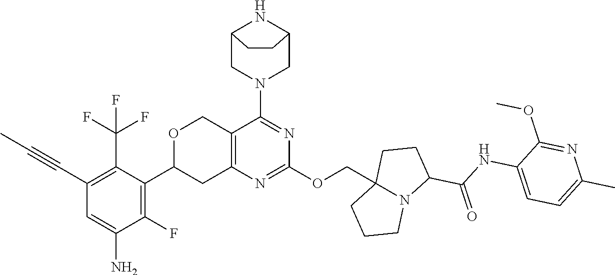 [SMILES: CC#Cc1cc(N)c(F)c(C2Cc3nc(OCC45CCCN4C(C(=O)Nc4ccc(C)nc4OC)CC5)nc(N4CC5CCC(C4)N5)c3CO2)c1C(F)(F)F]

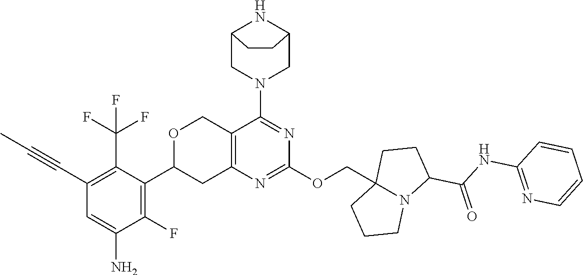 [SMILES: CC#Cc1cc(N)c(F)c(C2Cc3nc(OCC45CCCN4C(C(=O)Nc4ccccn4)CC5)nc(N4CC5CCC(C4)N5)c3CO2)c1C(F)(F)F]

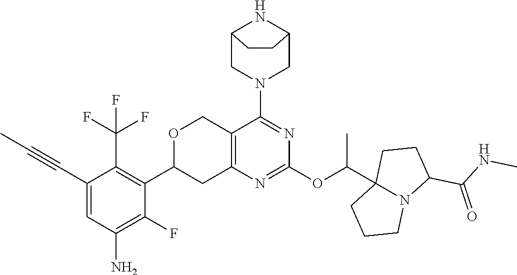 [SMILES: CC#Cc1cc(N)c(F)c(C2Cc3nc(OC(C)C45CCCN4C(C(=O)NC)CC5)nc(N4CC5CCC(C4)N5)c3CO2)c1C(F)(F)F]

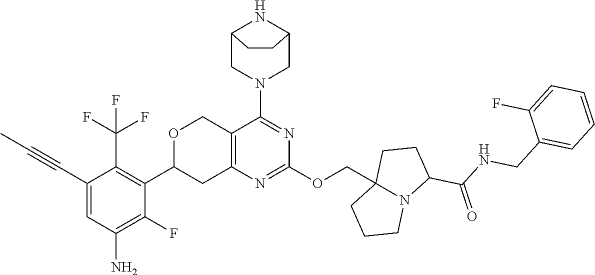 [SMILES: CC#Cc1cc(N)c(F)c(C2Cc3nc(OCC45CCCN4C(C(=O)NCc4ccccc4F)CC5)nc(N4CC5CCC(C4)N5)c3CO2)c1C(F)(F)F]

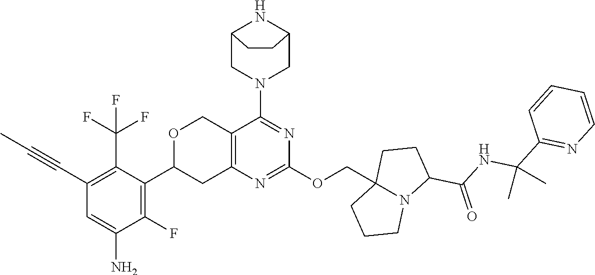 [SMILES: CC#Cc1cc(N)c(F)c(C2Cc3nc(OCC45CCCN4C(C(=O)NC(C)(C)c4ccccn4)CC5)nc(N4CC5CCC(C4)N5)c3CO2)c1C(F)(F)F]

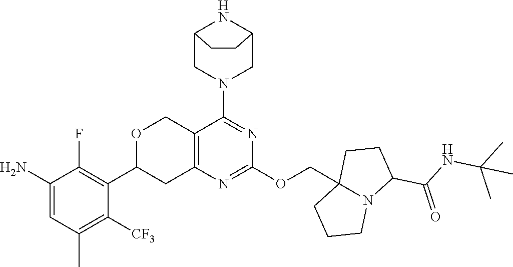 [SMILES: Cc1cc(N)c(F)c(C2Cc3nc(OCC45CCCN4C(C(=O)NC(C)(C)C)CC5)nc(N4CC5CCC(C4)N5)c3CO2)c1C(F)(F)F]

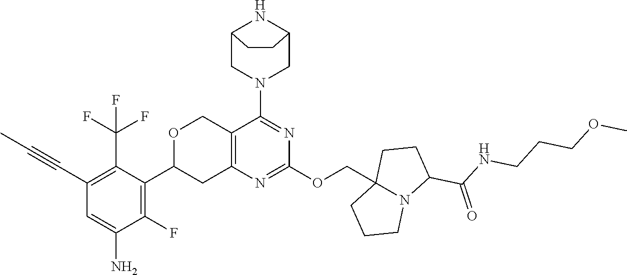 [SMILES: CC#Cc1cc(N)c(F)c(C2Cc3nc(OCC45CCCN4C(C(=O)NCCCOC)CC5)nc(N4CC5CCC(C4)N5)c3CO2)c1C(F)(F)F]

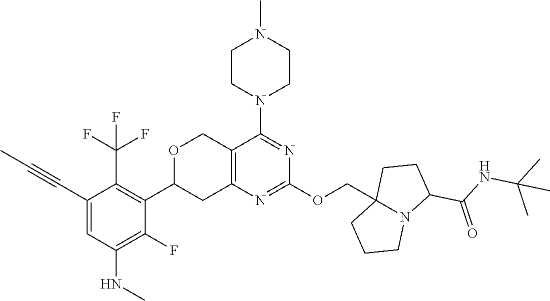 [SMILES: CC#Cc1cc(NC)c(F)c(C2Cc3nc(OCC45CCCN4C(C(=O)NC(C)(C)C)CC5)nc(N4CCN(C)CC4)c3CO2)c1C(F)(F)F]

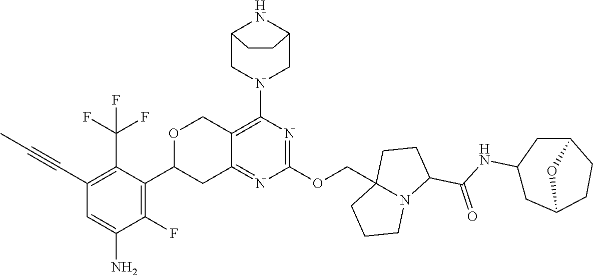 [SMILES: CC#Cc1cc(N)c(F)c(C2Cc3nc(OCC45CCCN4C(C(=O)NC4C[C@H]6CC[C@@H](C4)O6)CC5)nc(N4CC5CCC(C4)N5)c3CO2)c1C(F)(F)F]

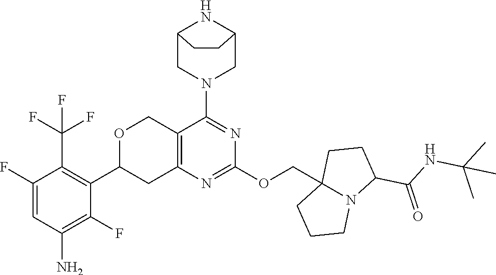 [SMILES: CC(C)(C)NC(=O)C1CCC2(COc3nc4c(c(N5CC6CCC(C5)N6)n3)COC(c3c(F)c(N)cc(F)c3C(F)(F)F)C4)CCCN12]

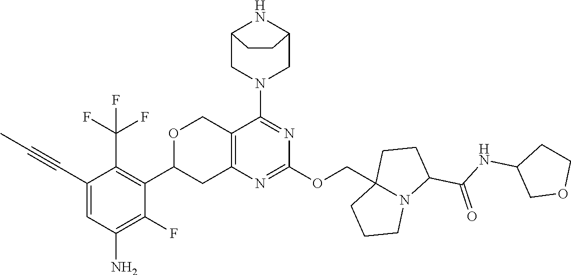 [SMILES: CC#Cc1cc(N)c(F)c(C2Cc3nc(OCC45CCCN4C(C(=O)NC4CCOC4)CC5)nc(N4CC5CCC(C4)N5)c3CO2)c1C(F)(F)F]

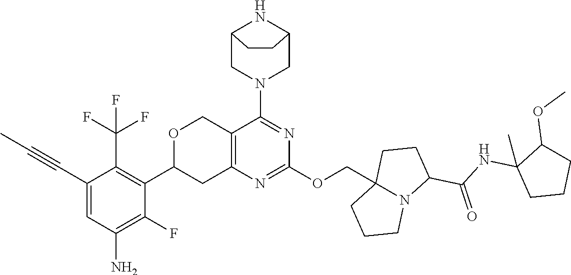 [SMILES: CC#Cc1cc(N)c(F)c(C2Cc3nc(OCC45CCCN4C(C(=O)NC4(C)CCCC4OC)CC5)nc(N4CC5CCC(C4)N5)c3CO2)c1C(F)(F)F]

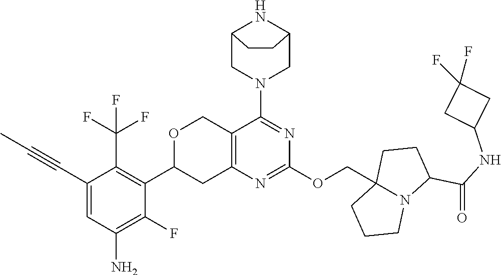 [SMILES: CC#Cc1cc(N)c(F)c(C2Cc3nc(OCC45CCCN4C(C(=O)NC4CC(F)(F)C4)CC5)nc(N4CC5CCC(C4)N5)c3CO2)c1C(F)(F)F]

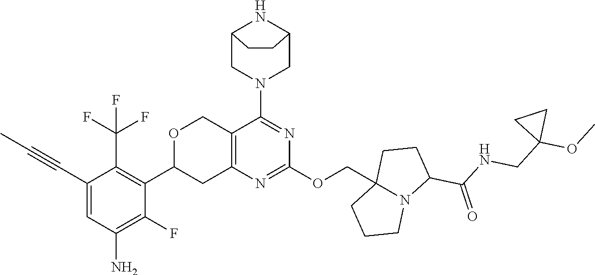 [SMILES: CC#Cc1cc(N)c(F)c(C2Cc3nc(OCC45CCCN4C(C(=O)NCC4(OC)CC4)CC5)nc(N4CC5CCC(C4)N5)c3CO2)c1C(F)(F)F]

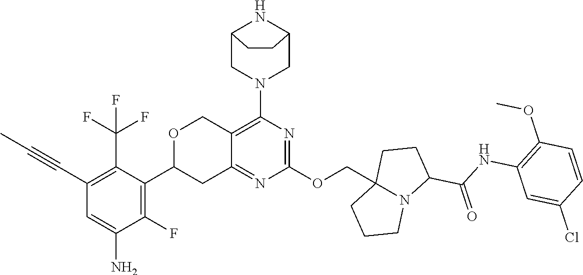 [SMILES: CC#Cc1cc(N)c(F)c(C2Cc3nc(OCC45CCCN4C(C(=O)Nc4cc(Cl)ccc4OC)CC5)nc(N4CC5CCC(C4)N5)c3CO2)c1C(F)(F)F]

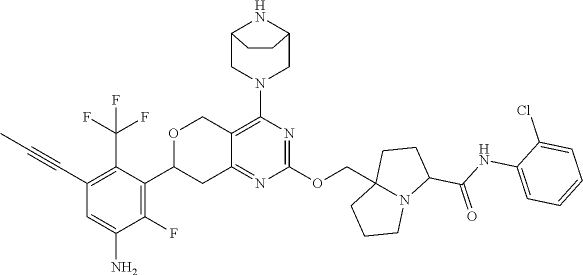 [SMILES: CC#Cc1cc(N)c(F)c(C2Cc3nc(OCC45CCCN4C(C(=O)Nc4ccccc4Cl)CC5)nc(N4CC5CCC(C4)N5)c3CO2)c1C(F)(F)F]

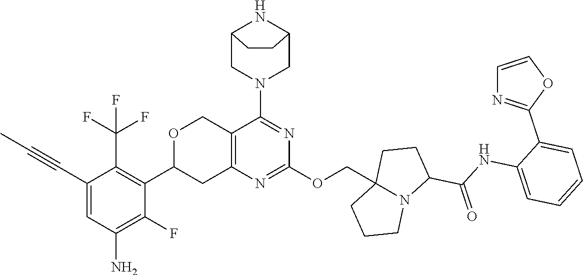 [SMILES: CC#Cc1cc(N)c(F)c(C2Cc3nc(OCC45CCCN4C(C(=O)Nc4ccccc4-c4ncco4)CC5)nc(N4CC5CCC(C4)N5)c3CO2)c1C(F)(F)F]

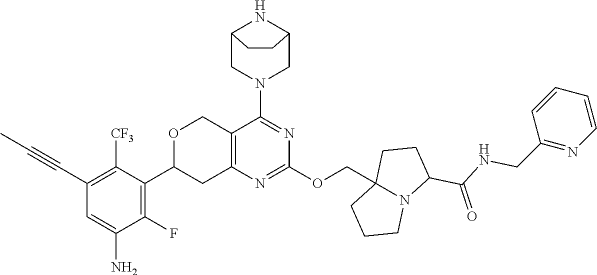 [SMILES: CC#Cc1cc(N)c(F)c(C2Cc3nc(OCC45CCCN4C(C(=O)NCc4ccccn4)CC5)nc(N4CC5CCC(C4)N5)c3CO2)c1C(F)(F)F]

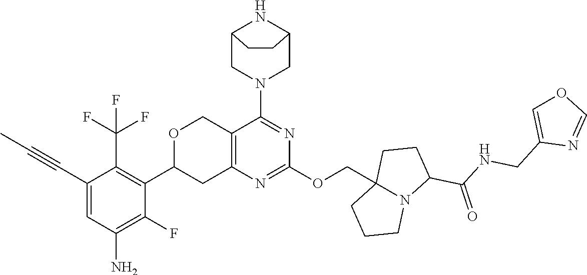 [SMILES: CC#Cc1cc(N)c(F)c(C2Cc3nc(OCC45CCCN4C(C(=O)NCc4cocn4)CC5)nc(N4CC5CCC(C4)N5)c3CO2)c1C(F)(F)F]

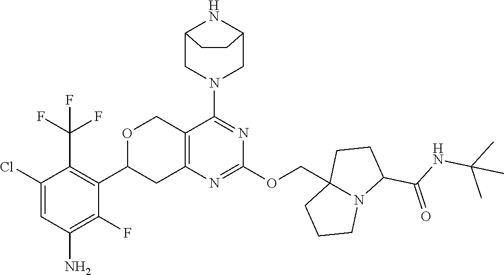 [SMILES: CC(C)(C)NC(=O)C1CCC2(COc3nc4c(c(N5CC6CCC(C5)N6)n3)COC(c3c(F)c(N)cc(Cl)c3C(F)(F)F)C4)CCCN12]

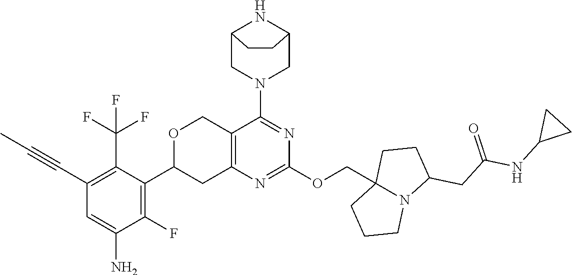 [SMILES: CC#Cc1cc(N)c(F)c(C2Cc3nc(OCC45CCCN4C(CC(=O)NC4CC4)CC5)nc(N4CC5CCC(C4)N5)c3CO2)c1C(F)(F)F]

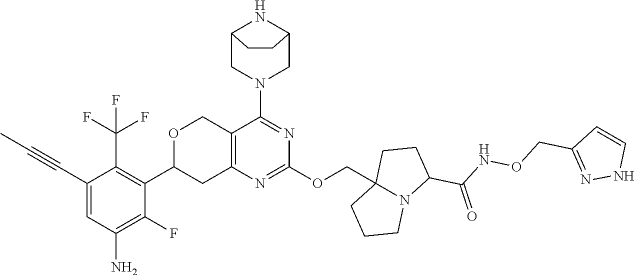 [SMILES: CC#Cc1cc(N)c(F)c(C2Cc3nc(OCC45CCCN4C(C(=O)NOCc4cc[nH]n4)CC5)nc(N4CC5CCC(C4)N5)c3CO2)c1C(F)(F)F]

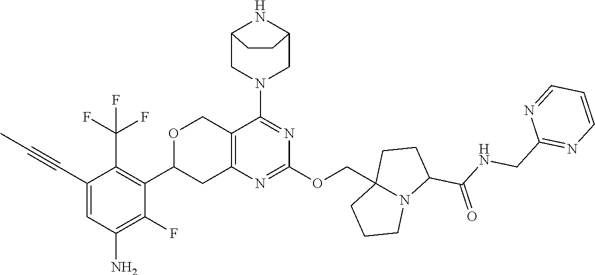 [SMILES: CC#Cc1cc(N)c(F)c(C2Cc3nc(OCC45CCCN4C(C(=O)NCc4ncccn4)CC5)nc(N4CC5CCC(C4)N5)c3CO2)c1C(F)(F)F]

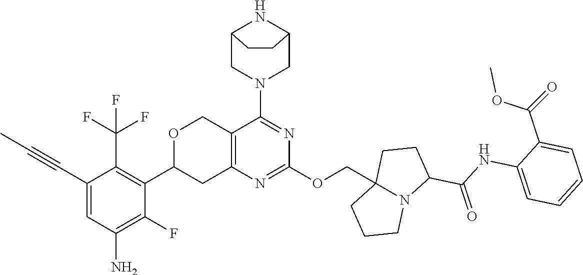 [SMILES: CC#Cc1cc(N)c(F)c(C2Cc3nc(OCC45CCCN4C(C(=O)Nc4ccccc4C(=O)OC)CC5)nc(N4CC5CCC(C4)N5)c3CO2)c1C(F)(F)F]

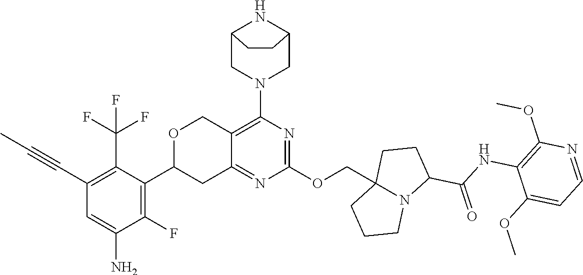 [SMILES: CC#Cc1cc(N)c(F)c(C2Cc3nc(OCC45CCCN4C(C(=O)Nc4c(OC)ccnc4OC)CC5)nc(N4CC5CCC(C4)N5)c3CO2)c1C(F)(F)F]

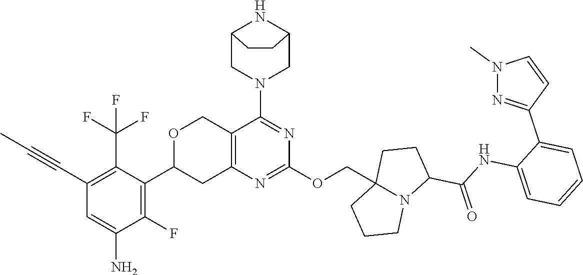 [SMILES: CC#Cc1cc(N)c(F)c(C2Cc3nc(OCC45CCCN4C(C(=O)Nc4ccccc4-c4ccn(C)n4)CC5)nc(N4CC5CCC(C4)N5)c3CO2)c1C(F)(F)F]

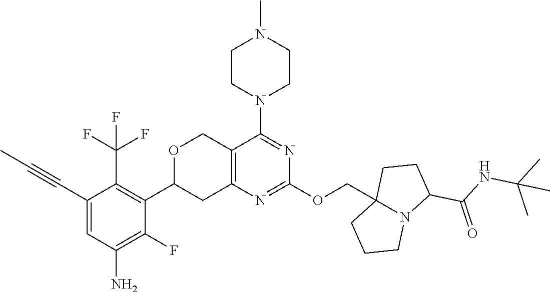 [SMILES: CC#Cc1cc(N)c(F)c(C2Cc3nc(OCC45CCCN4C(C(=O)NC(C)(C)C)CC5)nc(N4CCN(C)CC4)c3CO2)c1C(F)(F)F]

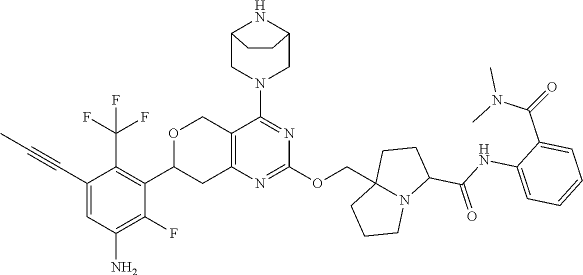 [SMILES: CC#Cc1cc(N)c(F)c(C2Cc3nc(OCC45CCCN4C(C(=O)Nc4ccccc4C(=O)N(C)C)CC5)nc(N4CC5CCC(C4)N5)c3CO2)c1C(F)(F)F]

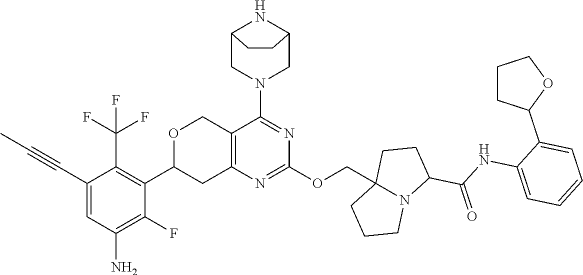 [SMILES: CC#Cc1cc(N)c(F)c(C2Cc3nc(OCC45CCCN4C(C(=O)Nc4ccccc4C4CCCO4)CC5)nc(N4CC5CCC(C4)N5)c3CO2)c1C(F)(F)F]

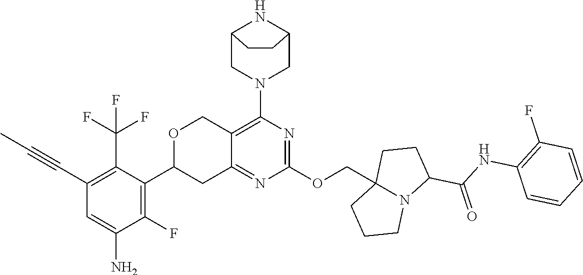 [SMILES: CC#Cc1cc(N)c(F)c(C2Cc3nc(OCC45CCCN4C(C(=O)Nc4ccccc4F)CC5)nc(N4CC5CCC(C4)N5)c3CO2)c1C(F)(F)F]

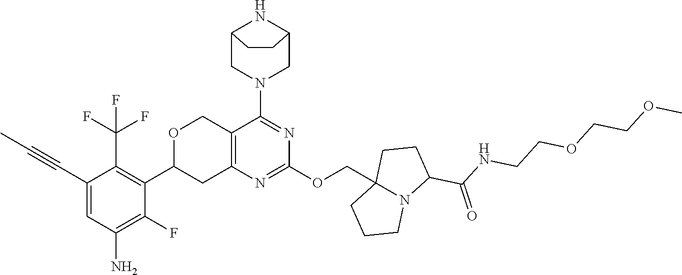 [SMILES: CC#Cc1cc(N)c(F)c(C2Cc3nc(OCC45CCCN4C(C(=O)NCCOCCOC)CC5)nc(N4CC5CCC(C4)N5)c3CO2)c1C(F)(F)F]

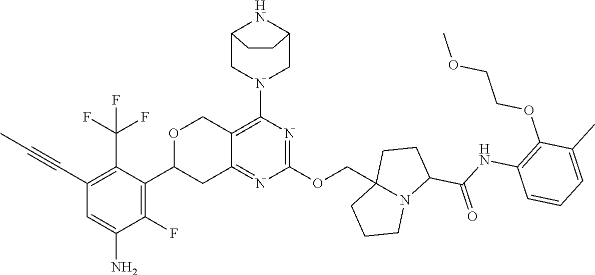 [SMILES: CC#Cc1cc(N)c(F)c(C2Cc3nc(OCC45CCCN4C(C(=O)Nc4cccc(C)c4OCCOC)CC5)nc(N4CC5CCC(C4)N5)c3CO2)c1C(F)(F)F]

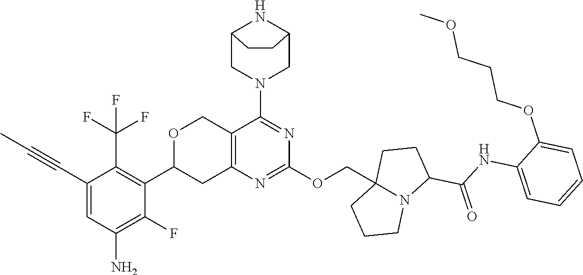 [SMILES: CC#Cc1cc(N)c(F)c(C2Cc3nc(OCC45CCCN4C(C(=O)Nc4ccccc4OCCCOC)CC5)nc(N4CC5CCC(C4)N5)c3CO2)c1C(F)(F)F]

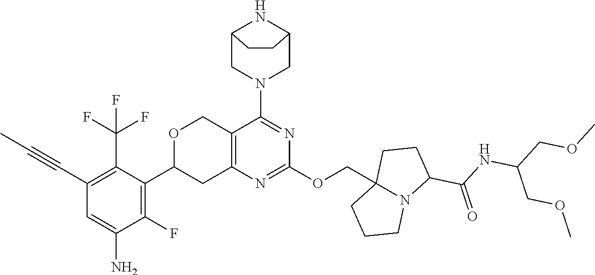 [SMILES: CC#Cc1cc(N)c(F)c(C2Cc3nc(OCC45CCCN4C(C(=O)NC(COC)COC)CC5)nc(N4CC5CCC(C4)N5)c3CO2)c1C(F)(F)F]

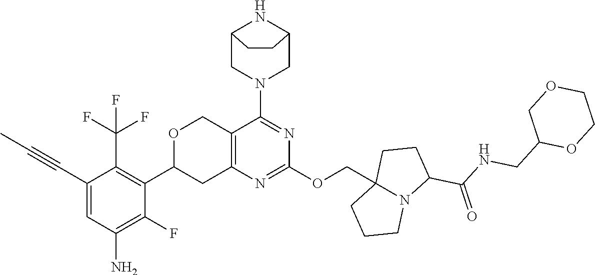 [SMILES: CC#Cc1cc(N)c(F)c(C2Cc3nc(OCC45CCCN4C(C(=O)NCC4COCCO4)CC5)nc(N4CC5CCC(C4)N5)c3CO2)c1C(F)(F)F]

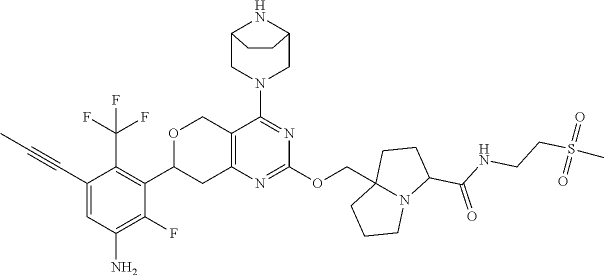 [SMILES: CC#Cc1cc(N)c(F)c(C2Cc3nc(OCC45CCCN4C(C(=O)NCCS(C)(=O)=O)CC5)nc(N4CC5CCC(C4)N5)c3CO2)c1C(F)(F)F]

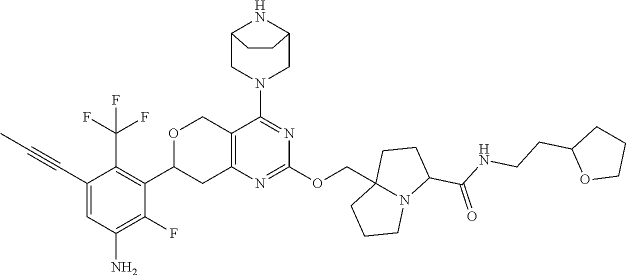 [SMILES: CC#Cc1cc(N)c(F)c(C2Cc3nc(OCC45CCCN4C(C(=O)NCCC4CCCO4)CC5)nc(N4CC5CCC(C4)N5)c3CO2)c1C(F)(F)F]

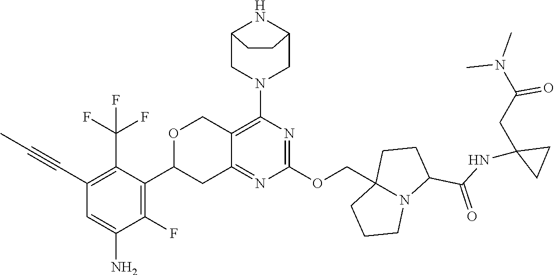 [SMILES: CC#Cc1cc(N)c(F)c(C2Cc3nc(OCC45CCCN4C(C(=O)NC4(CC(=O)N(C)C)CC4)CC5)nc(N4CC5CCC(C4)N5)c3CO2)c1C(F)(F)F]

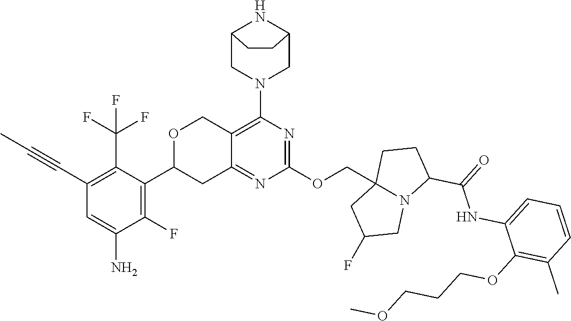 [SMILES: CC#Cc1cc(N)c(F)c(C2Cc3nc(OCC45CCC(C(=O)Nc6cccc(C)c6OCCCOC)N4CC(F)C5)nc(N4CC5CCC(C4)N5)c3CO2)c1C(F)(F)F]

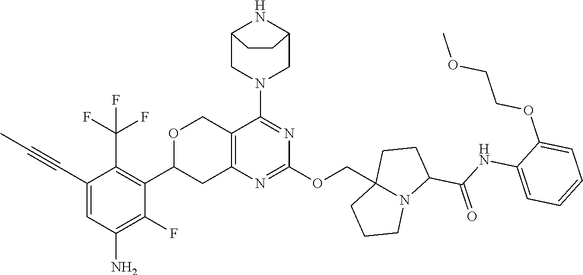 [SMILES: CC#Cc1cc(N)c(F)c(C2Cc3nc(OCC45CCCN4C(C(=O)Nc4ccccc4OCCOC)CC5)nc(N4CC5CCC(C4)N5)c3CO2)c1C(F)(F)F]

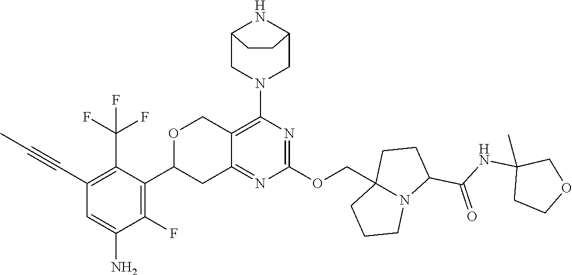 [SMILES: CC#Cc1cc(N)c(F)c(C2Cc3nc(OCC45CCCN4C(C(=O)NC4(C)CCOC4)CC5)nc(N4CC5CCC(C4)N5)c3CO2)c1C(F)(F)F]

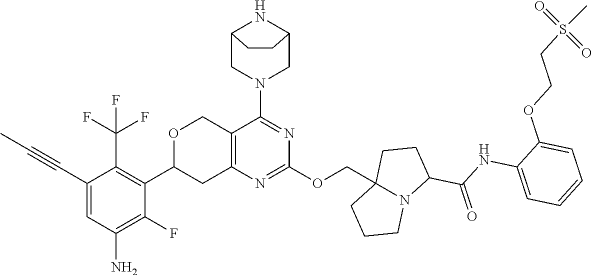 [SMILES: CC#Cc1cc(N)c(F)c(C2Cc3nc(OCC45CCCN4C(C(=O)Nc4ccccc4OCCS(C)(=O)=O)CC5)nc(N4CC5CCC(C4)N5)c3CO2)c1C(F)(F)F]

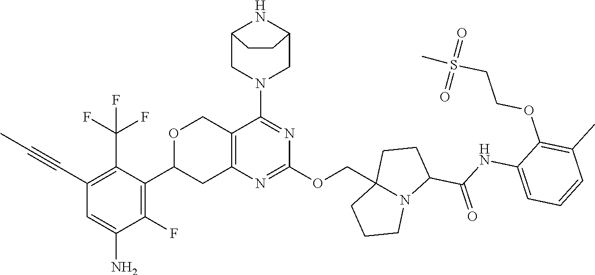 [SMILES: CC#Cc1cc(N)c(F)c(C2Cc3nc(OCC45CCCN4C(C(=O)Nc4cccc(C)c4OCCS(C)(=O)=O)CC5)nc(N4CC5CCC(C4)N5)c3CO2)c1C(F)(F)F]

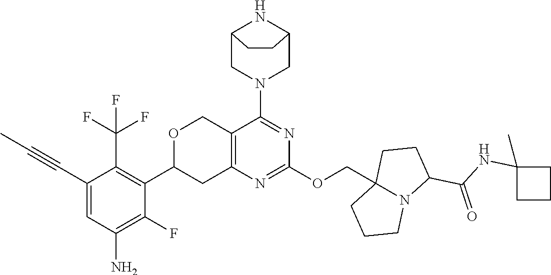 [SMILES: CC#Cc1cc(N)c(F)c(C2Cc3nc(OCC45CCCN4C(C(=O)NC4(C)CCC4)CC5)nc(N4CC5CCC(C4)N5)c3CO2)c1C(F)(F)F]

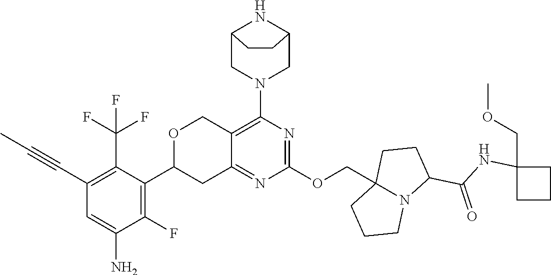 [SMILES: CC#Cc1cc(N)c(F)c(C2Cc3nc(OCC45CCCN4C(C(=O)NC4(COC)CCC4)CC5)nc(N4CC5CCC(C4)N5)c3CO2)c1C(F)(F)F]

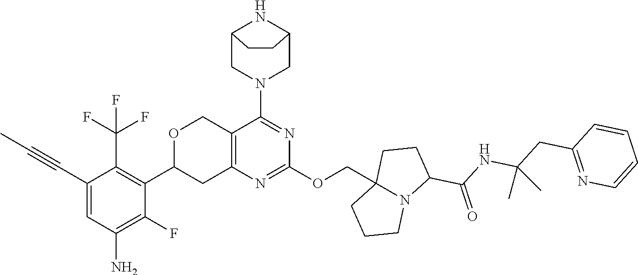 [SMILES: CC#Cc1cc(N)c(F)c(C2Cc3nc(OCC45CCCN4C(C(=O)NC(C)(C)Cc4ccccn4)CC5)nc(N4CC5CCC(C4)N5)c3CO2)c1C(F)(F)F]

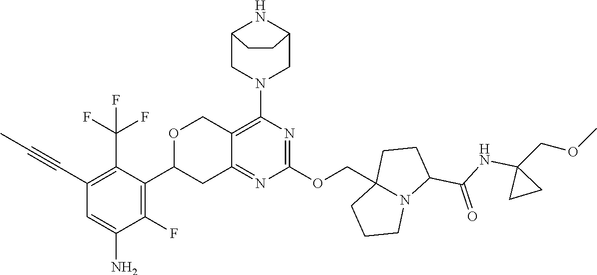 [SMILES: CC#Cc1cc(N)c(F)c(C2Cc3nc(OCC45CCCN4C(C(=O)NC4(COC)CC4)CC5)nc(N4CC5CCC(C4)N5)c3CO2)c1C(F)(F)F]

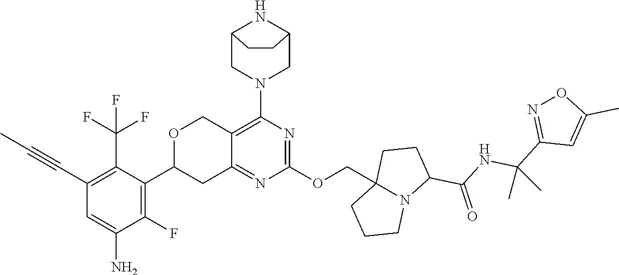 [SMILES: CC#Cc1cc(N)c(F)c(C2Cc3nc(OCC45CCCN4C(C(=O)NC(C)(C)c4cc(C)on4)CC5)nc(N4CC5CCC(C4)N5)c3CO2)c1C(F)(F)F]

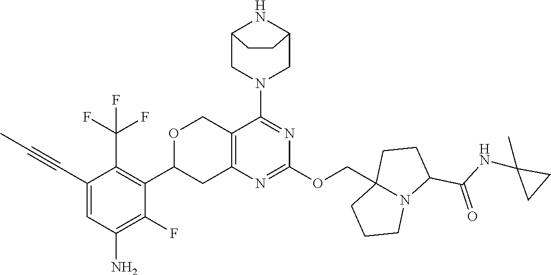 [SMILES: CC#Cc1cc(N)c(F)c(C2Cc3nc(OCC45CCCN4C(C(=O)NC4(C)CC4)CC5)nc(N4CC5CCC(C4)N5)c3CO2)c1C(F)(F)F]

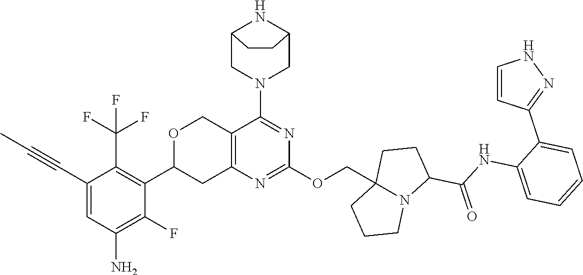 [SMILES: CC#Cc1cc(N)c(F)c(C2Cc3nc(OCC45CCCN4C(C(=O)Nc4ccccc4-c4cc[nH]n4)CC5)nc(N4CC5CCC(C4)N5)c3CO2)c1C(F)(F)F]

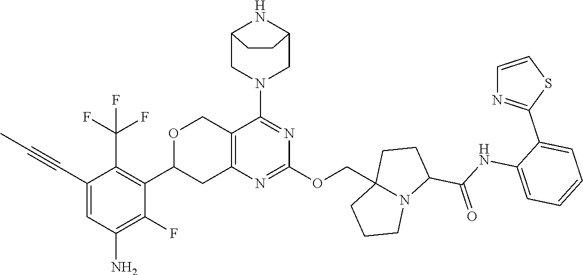 [SMILES: CC#Cc1cc(N)c(F)c(C2Cc3nc(OCC45CCCN4C(C(=O)Nc4ccccc4-c4nccs4)CC5)nc(N4CC5CCC(C4)N5)c3CO2)c1C(F)(F)F]

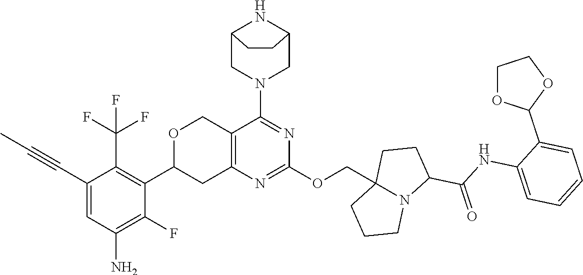 [SMILES: CC#Cc1cc(N)c(F)c(C2Cc3nc(OCC45CCCN4C(C(=O)Nc4ccccc4C4OCCO4)CC5)nc(N4CC5CCC(C4)N5)c3CO2)c1C(F)(F)F]

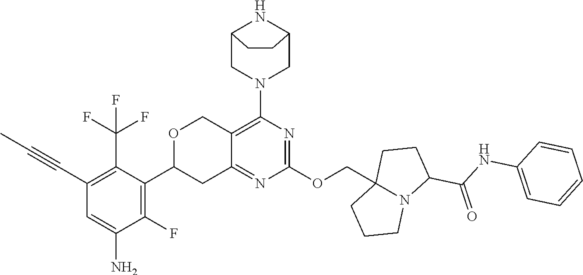 [SMILES: CC#Cc1cc(N)c(F)c(C2Cc3nc(OCC45CCCN4C(C(=O)Nc4ccccc4)CC5)nc(N4CC5CCC(C4)N5)c3CO2)c1C(F)(F)F]

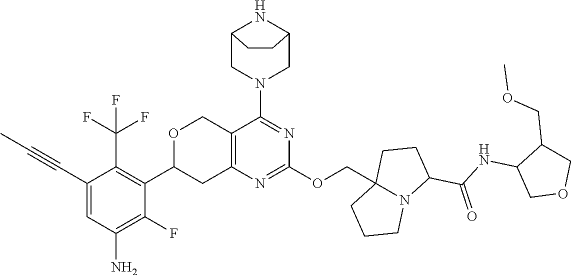 [SMILES: CC#Cc1cc(N)c(F)c(C2Cc3nc(OCC45CCCN4C(C(=O)NC4COCC4COC)CC5)nc(N4CC5CCC(C4)N5)c3CO2)c1C(F)(F)F]

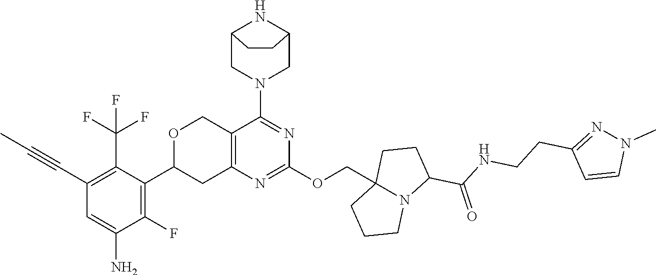 [SMILES: CC#Cc1cc(N)c(F)c(C2Cc3nc(OCC45CCCN4C(C(=O)NCCc4ccn(C)n4)CC5)nc(N4CC5CCC(C4)N5)c3CO2)c1C(F)(F)F]

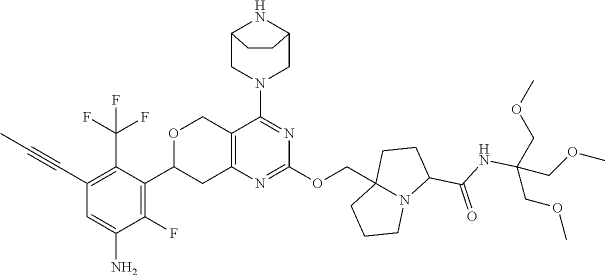 [SMILES: CC#Cc1cc(N)c(F)c(C2Cc3nc(OCC45CCCN4C(C(=O)NC(COC)(COC)COC)CC5)nc(N4CC5CCC(C4)N5)c3CO2)c1C(F)(F)F]

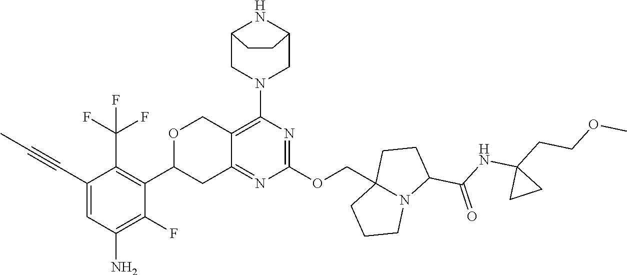 [SMILES: CC#Cc1cc(N)c(F)c(C2Cc3nc(OCC45CCCN4C(C(=O)NC4(CCOC)CC4)CC5)nc(N4CC5CCC(C4)N5)c3CO2)c1C(F)(F)F]

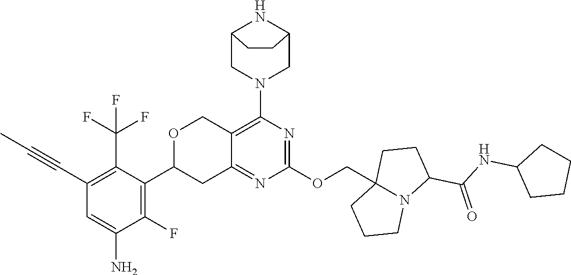 [SMILES: CC#Cc1cc(N)c(F)c(C2Cc3nc(OCC45CCCN4C(C(=O)NC4CCCC4)CC5)nc(N4CC5CCC(C4)N5)c3CO2)c1C(F)(F)F]

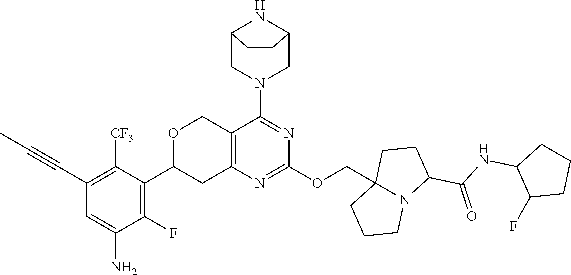 [SMILES: CC#Cc1cc(N)c(F)c(C2Cc3nc(OCC45CCCN4C(C(=O)NC4CCCC4F)CC5)nc(N4CC5CCC(C4)N5)c3CO2)c1C(F)(F)F]

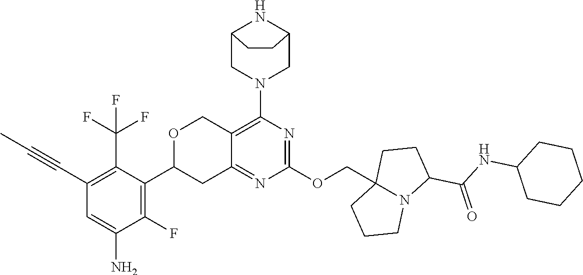 [SMILES: CC#Cc1cc(N)c(F)c(C2Cc3nc(OCC45CCCN4C(C(=O)NC4CCCCC4)CC5)nc(N4CC5CCC(C4)N5)c3CO2)c1C(F)(F)F]

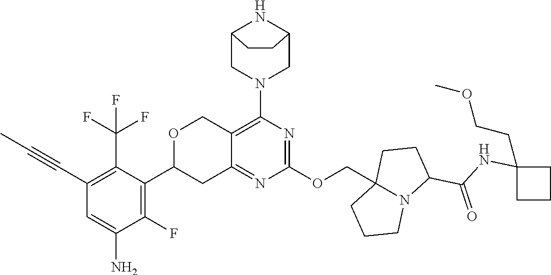 [SMILES: CC#Cc1cc(N)c(F)c(C2Cc3nc(OCC45CCCN4C(C(=O)NC4(CCOC)CCC4)CC5)nc(N4CC5CCC(C4)N5)c3CO2)c1C(F)(F)F]

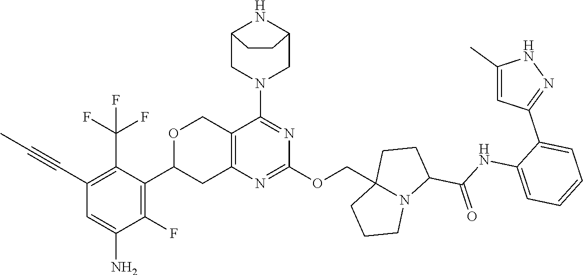 [SMILES: CC#Cc1cc(N)c(F)c(C2Cc3nc(OCC45CCCN4C(C(=O)Nc4ccccc4-c4cc(C)[nH]n4)CC5)nc(N4CC5CCC(C4)N5)c3CO2)c1C(F)(F)F]

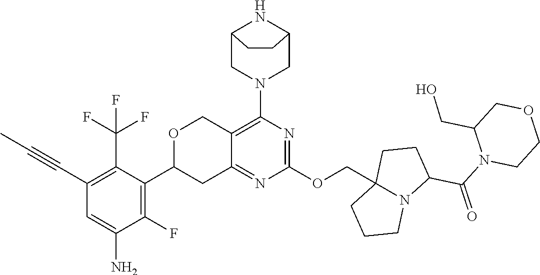 [SMILES: CC#Cc1cc(N)c(F)c(C2Cc3nc(OCC45CCCN4C(C(=O)N4CCOCC4CO)CC5)nc(N4CC5CCC(C4)N5)c3CO2)c1C(F)(F)F]

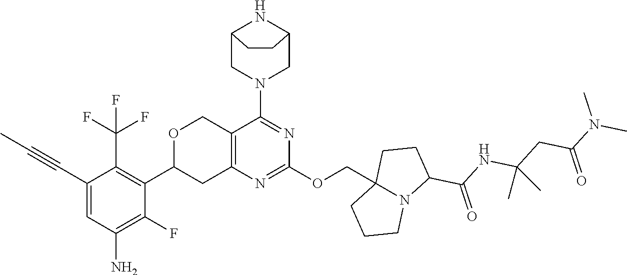 [SMILES: CC#Cc1cc(N)c(F)c(C2Cc3nc(OCC45CCCN4C(C(=O)NC(C)(C)CC(=O)N(C)C)CC5)nc(N4CC5CCC(C4)N5)c3CO2)c1C(F)(F)F]

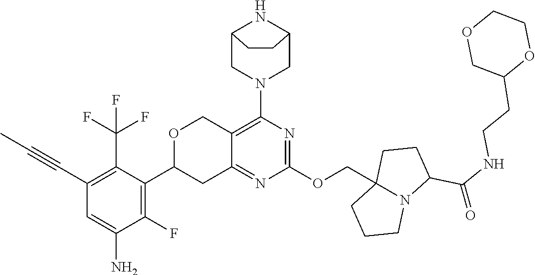 [SMILES: CC#Cc1cc(N)c(F)c(C2Cc3nc(OCC45CCCN4C(C(=O)NCCC4COCCO4)CC5)nc(N4CC5CCC(C4)N5)c3CO2)c1C(F)(F)F]

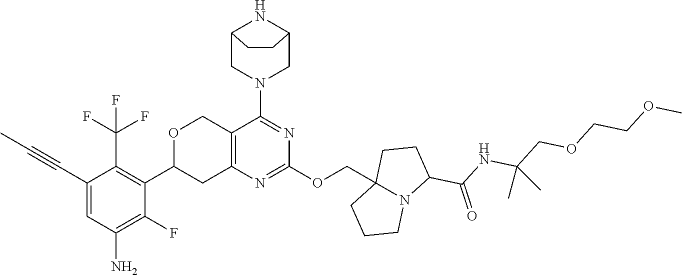 [SMILES: CC#Cc1cc(N)c(F)c(C2Cc3nc(OCC45CCCN4C(C(=O)NC(C)(C)COCCOC)CC5)nc(N4CC5CCC(C4)N5)c3CO2)c1C(F)(F)F]

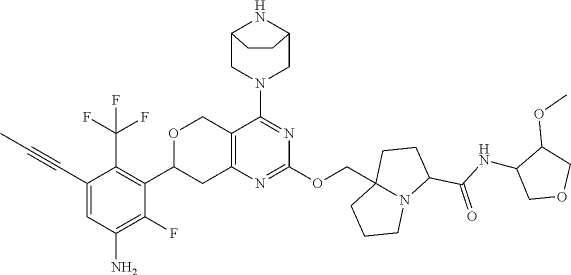 [SMILES: CC#Cc1cc(N)c(F)c(C2Cc3nc(OCC45CCCN4C(C(=O)NC4COCC4OC)CC5)nc(N4CC5CCC(C4)N5)c3CO2)c1C(F)(F)F]

TABLE 2a

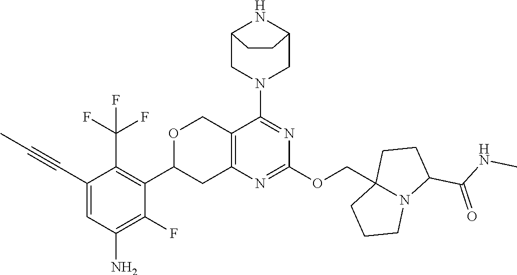 [SMILES: CC#Cc1cc(N)c(F)c(C2Cc3nc(OCC45CCCN4C(C(=O)NC)CC5)nc(N4CC5CCC(C4)N5)c3CO2)c1C(F)(F)F]

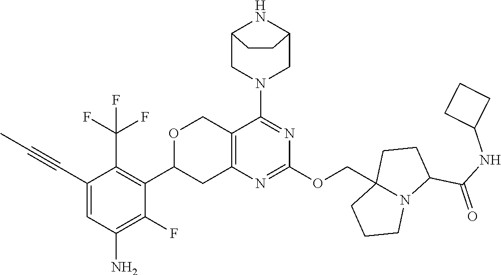 [SMILES: CC#Cc1cc(N)c(F)c(C2Cc3nc(OCC45CCCN4C(C(=O)NC4CCC4)CC5)nc(N4CC5CCC(C4)N5)c3CO2)c1C(F)(F)F]

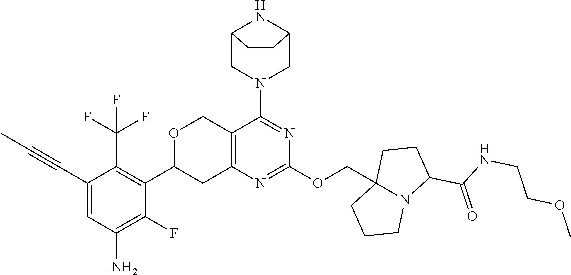 [SMILES: CC#Cc1cc(N)c(F)c(C2Cc3nc(OCC45CCCN4C(C(=O)NCCOC)CC5)nc(N4CC5CCC(C4)N5)c3CO2)c1C(F)(F)F]

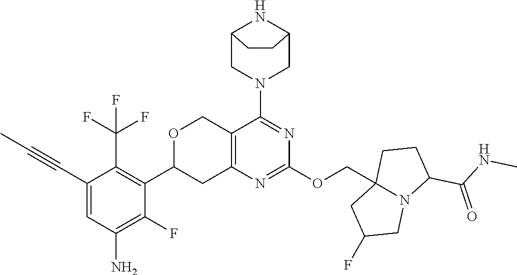 [SMILES: CC#Cc1cc(N)c(F)c(C2Cc3nc(OCC45CCC(C(=O)NC)N4CC(F)C5)nc(N4CC5CCC(C4)N5)c3CO2)c1C(F)(F)F]

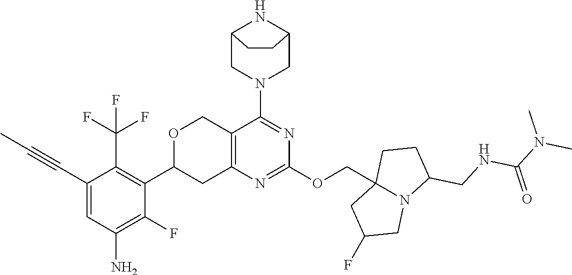 [SMILES: CC#Cc1cc(N)c(F)c(C2Cc3nc(OCC45CCC(CNC(=O)N(C)C)N4CC(F)C5)nc(N4CC5CCC(C4)N5)c3CO2)c1C(F)(F)F]

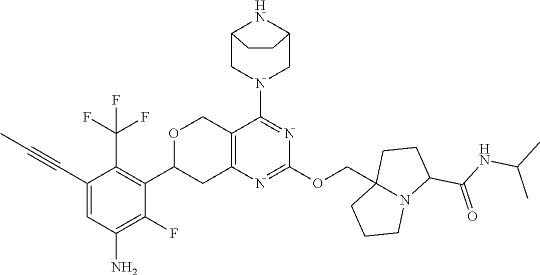 [SMILES: CC#Cc1cc(N)c(F)c(C2Cc3nc(OCC45CCCN4C(C(=O)NC(C)C)CC5)nc(N4CC5CCC(C4)N5)c3CO2)c1C(F)(F)F]

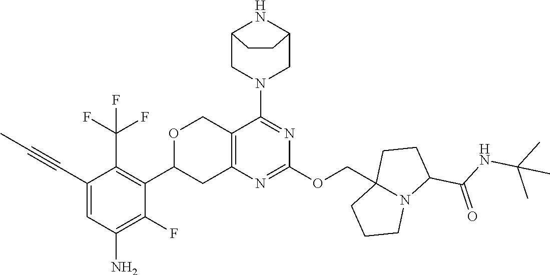 [SMILES: CC#Cc1cc(N)c(F)c(C2Cc3nc(OCC45CCCN4C(C(=O)NC(C)(C)C)CC5)nc(N4CC5CCC(C4)N5)c3CO2)c1C(F)(F)F]

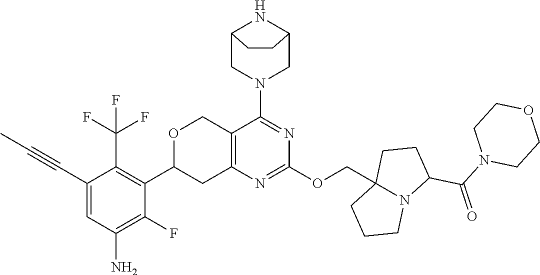 [SMILES: CC#Cc1cc(N)c(F)c(C2Cc3nc(OCC45CCCN4C(C(=O)N4CCOCC4)CC5)nc(N4CC5CCC(C4)N5)c3CO2)c1C(F)(F)F]

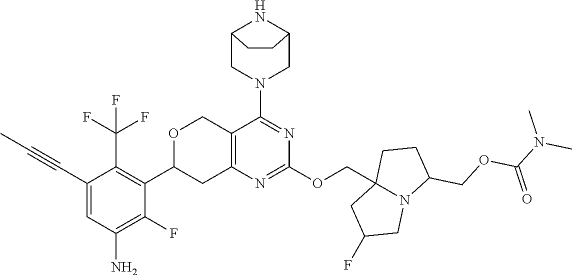 [SMILES: CC#Cc1cc(N)c(F)c(C2Cc3nc(OCC45CCC(COC(=O)N(C)C)N4CC(F)C5)nc(N4CC5CCC(C4)N5)c3CO2)c1C(F)(F)F]

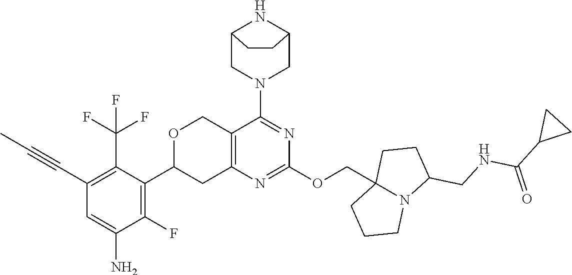 [SMILES: CC#Cc1cc(N)c(F)c(C2Cc3nc(OCC45CCCN4C(CNC(=O)C4CC4)CC5)nc(N4CC5CCC(C4)N5)c3CO2)c1C(F)(F)F]

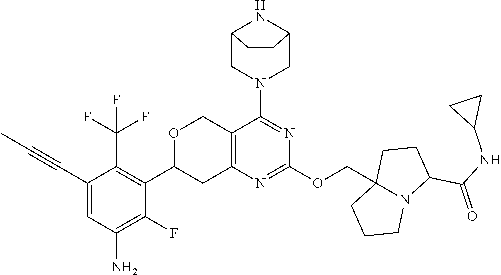 [SMILES: CC#Cc1cc(N)c(F)c(C2Cc3nc(OCC45CCCN4C(C(=O)NC4CC4)CC5)nc(N4CC5CCC(C4)N5)c3CO2)c1C(F)(F)F]

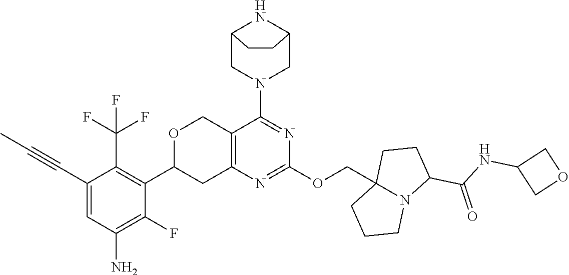 [SMILES: CC#Cc1cc(N)c(F)c(C2Cc3nc(OCC45CCCN4C(C(=O)NC4COC4)CC5)nc(N4CC5CCC(C4)N5)c3CO2)c1C(F)(F)F]

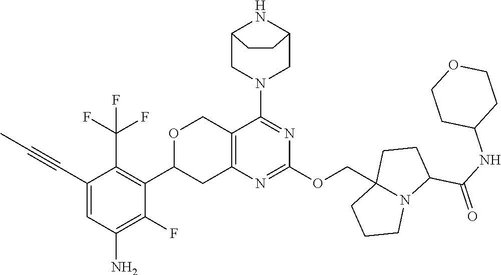 [SMILES: CC#Cc1cc(N)c(F)c(C2Cc3nc(OCC45CCCN4C(C(=O)NC4CCOCC4)CC5)nc(N4CC5CCC(C4)N5)c3CO2)c1C(F)(F)F]

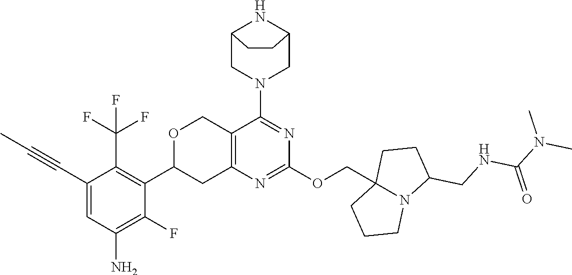 [SMILES: CC#Cc1cc(N)c(F)c(C2Cc3nc(OCC45CCCN4C(CNC(=O)N(C)C)CC5)nc(N4CC5CCC(C4)N5)c3CO2)c1C(F)(F)F]

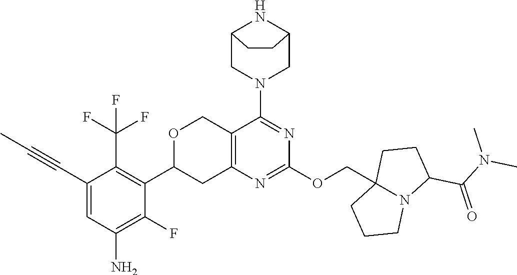 [SMILES: CC#Cc1cc(N)c(F)c(C2Cc3nc(OCC45CCCN4C(C(=O)N(C)C)CC5)nc(N4CC5CCC(C4)N5)c3CO2)c1C(F)(F)F]

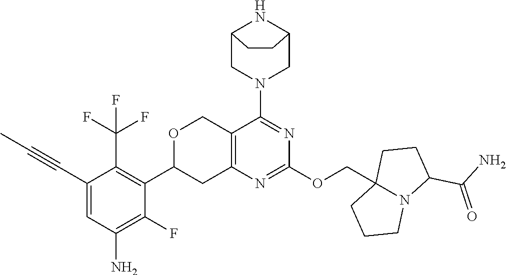 [SMILES: CC#Cc1cc(N)c(F)c(C2Cc3nc(OCC45CCCN4C(C(N)=O)CC5)nc(N4CC5CCC(C4)N5)c3CO2)c1C(F)(F)F]

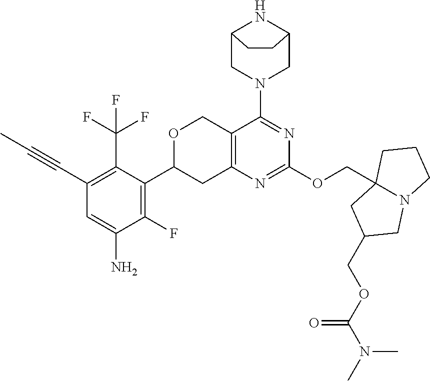 [SMILES: CC#Cc1cc(N)c(F)c(C2Cc3nc(OCC45CCCN4CC(COC(=O)N(C)C)C5)nc(N4CC5CCC(C4)N5)c3CO2)c1C(F)(F)F]

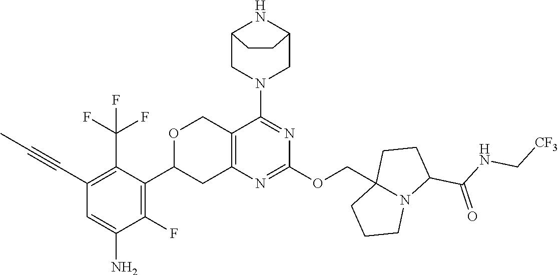 [SMILES: CC#Cc1cc(N)c(F)c(C2Cc3nc(OCC45CCCN4C(C(=O)NCC(F)(F)F)CC5)nc(N4CC5CCC(C4)N5)c3CO2)c1C(F)(F)F]

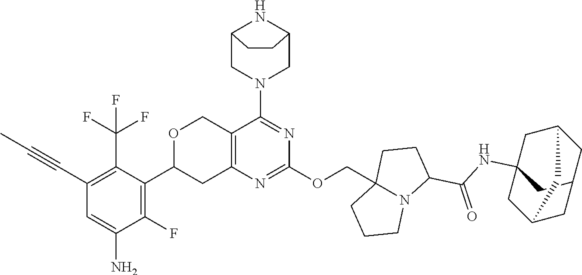 [SMILES: CC#Cc1cc(N)c(F)c(C2Cc3nc(OCC45CCCN4C(C(=O)N[C@]46CC7C[C@H](C[C@H](C7)C4)C6)CC5)nc(N4CC5CCC(C4)N5)c3CO2)c1C(F)(F)F]

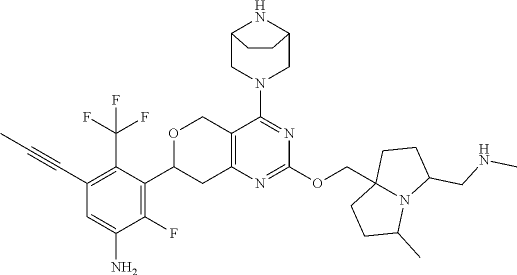 [SMILES: CC#Cc1cc(N)c(F)c(C2Cc3nc(OCC45CCC(C)N4C(CNC)CC5)nc(N4CC5CCC(C4)N5)c3CO2)c1C(F)(F)F]

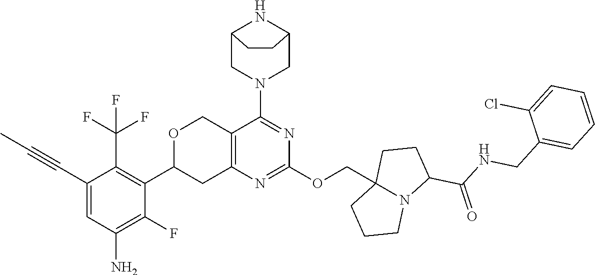 [SMILES: CC#Cc1cc(N)c(F)c(C2Cc3nc(OCC45CCCN4C(C(=O)NCc4ccccc4Cl)CC5)nc(N4CC5CCC(C4)N5)c3CO2)c1C(F)(F)F]

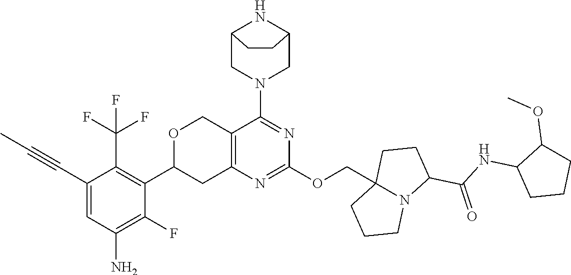 [SMILES: CC#Cc1cc(N)c(F)c(C2Cc3nc(OCC45CCCN4C(C(=O)NC4CCCC4OC)CC5)nc(N4CC5CCC(C4)N5)c3CO2)c1C(F)(F)F]

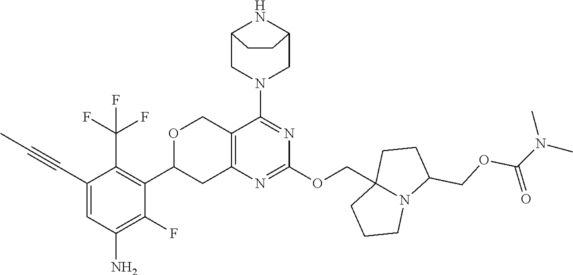 [SMILES: CC#Cc1cc(N)c(F)c(C2Cc3nc(OCC45CCCN4C(COC(=O)N(C)C)CC5)nc(N4CC5CCC(C4)N5)c3CO2)c1C(F)(F)F]

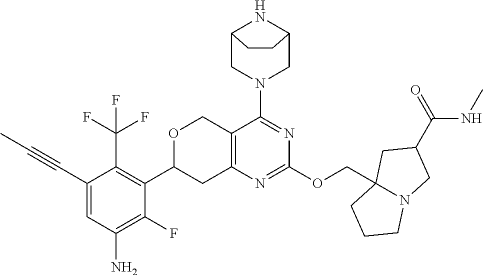 [SMILES: CC#Cc1cc(N)c(F)c(C2Cc3nc(OCC45CCCN4CC(C(=O)NC)C5)nc(N4CC5CCC(C4)N5)c3CO2)c1C(F)(F)F]

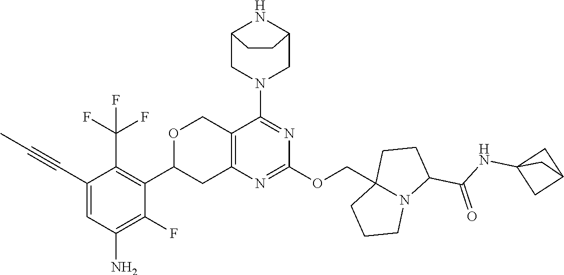 [SMILES: CC#Cc1cc(N)c(F)c(C2Cc3nc(OCC45CCCN4C(C(=O)NC46CC(C4)C6)CC5)nc(N4CC5CCC(C4)N5)c3CO2)c1C(F)(F)F]

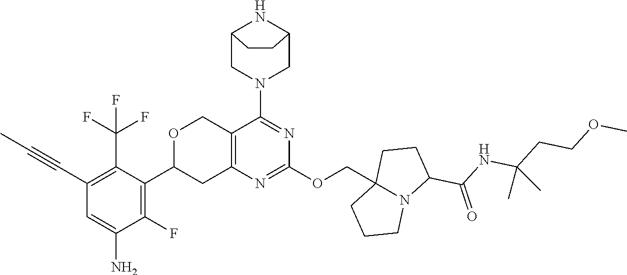 [SMILES: CC#Cc1cc(N)c(F)c(C2Cc3nc(OCC45CCCN4C(C(=O)NC(C)(C)CCOC)CC5)nc(N4CC5CCC(C4)N5)c3CO2)c1C(F)(F)F]

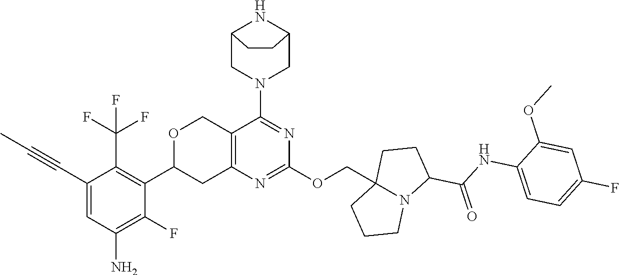 [SMILES: CC#Cc1cc(N)c(F)c(C2Cc3nc(OCC45CCCN4C(C(=O)Nc4ccc(F)cc4OC)CC5)nc(N4CC5CCC(C4)N5)c3CO2)c1C(F)(F)F]

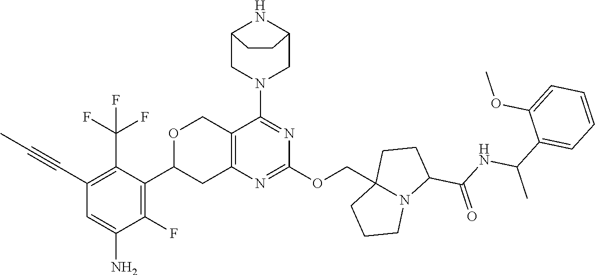 [SMILES: CC#Cc1cc(N)c(F)c(C2Cc3nc(OCC45CCCN4C(C(=O)NC(C)c4ccccc4OC)CC5)nc(N4CC5CCC(C4)N5)c3CO2)c1C(F)(F)F]

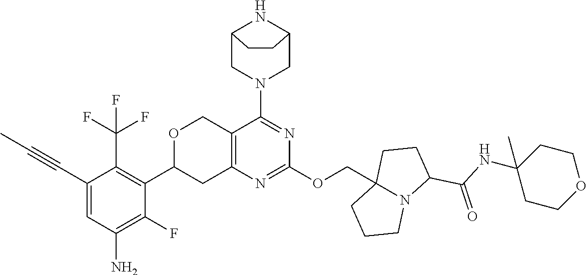 [SMILES: CC#Cc1cc(N)c(F)c(C2Cc3nc(OCC45CCCN4C(C(=O)NC4(C)CCOCC4)CC5)nc(N4CC5CCC(C4)N5)c3CO2)c1C(F)(F)F]

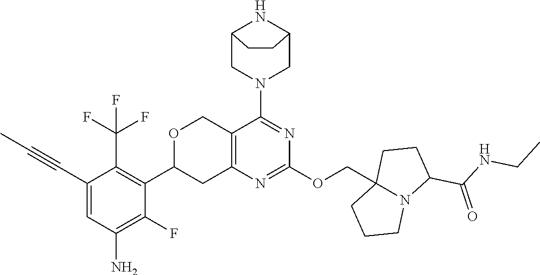 [SMILES: CC#Cc1cc(N)c(F)c(C2Cc3nc(OCC45CCCN4C(C(=O)NCC)CC5)nc(N4CC5CCC(C4)N5)c3CO2)c1C(F)(F)F]

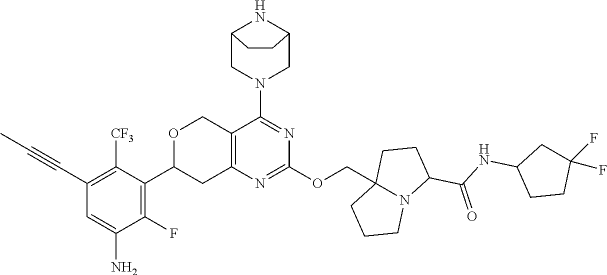 [SMILES: CC#Cc1cc(N)c(F)c(C2Cc3nc(OCC45CCCN4C(C(=O)NC4CCC(F)(F)C4)CC5)nc(N4CC5CCC(C4)N5)c3CO2)c1C(F)(F)F]

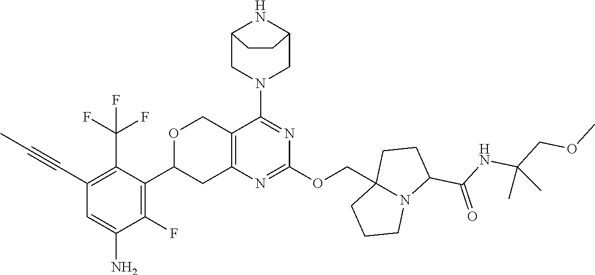 [SMILES: CC#Cc1cc(N)c(F)c(C2Cc3nc(OCC45CCCN4C(C(=O)NC(C)(C)COC)CC5)nc(N4CC5CCC(C4)N5)c3CO2)c1C(F)(F)F]

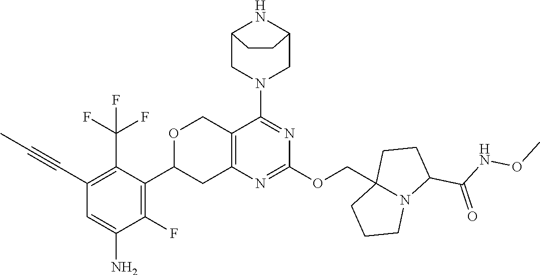 [SMILES: CC#Cc1cc(N)c(F)c(C2Cc3nc(OCC45CCCN4C(C(=O)NOC)CC5)nc(N4CC5CCC(C4)N5)c3CO2)c1C(F)(F)F]

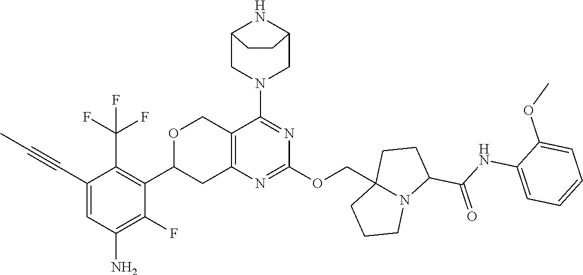 [SMILES: CC#Cc1cc(N)c(F)c(C2Cc3nc(OCC45CCCN4C(C(=O)Nc4ccccc4OC)CC5)nc(N4CC5CCC(C4)N5)c3CO2)c1C(F)(F)F]

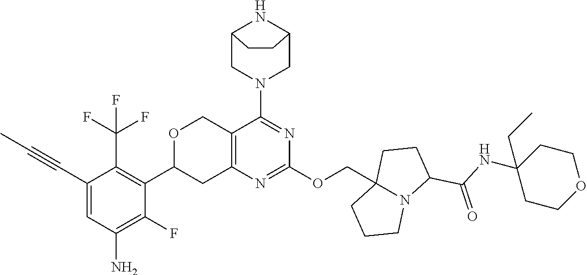 [SMILES: CC#Cc1cc(N)c(F)c(C2Cc3nc(OCC45CCCN4C(C(=O)NC4(CC)CCOCC4)CC5)nc(N4CC5CCC(C4)N5)c3CO2)c1C(F)(F)F]

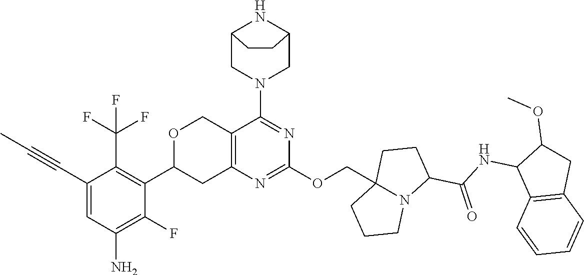 [SMILES: CC#Cc1cc(N)c(F)c(C2Cc3nc(OCC45CCCN4C(C(=O)NC4c6ccccc6CC4OC)CC5)nc(N4CC5CCC(C4)N5)c3CO2)c1C(F)(F)F]

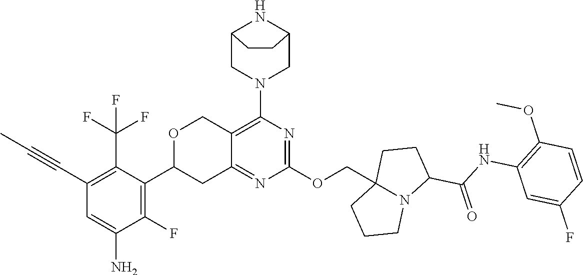 [SMILES: CC#Cc1cc(N)c(F)c(C2Cc3nc(OCC45CCCN4C(C(=O)Nc4cc(F)ccc4OC)CC5)nc(N4CC5CCC(C4)N5)c3CO2)c1C(F)(F)F]

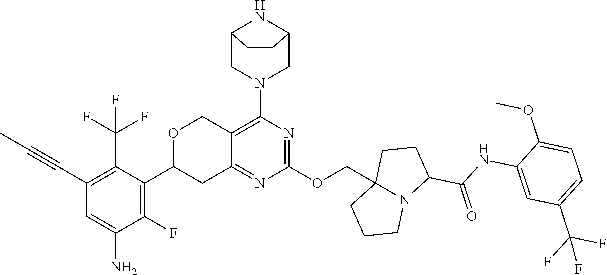 [SMILES: CC#Cc1cc(N)c(F)c(C2Cc3nc(OCC45CCCN4C(C(=O)Nc4cc(C(F)(F)F)ccc4OC)CC5)nc(N4CC5CCC(C4)N5)c3CO2)c1C(F)(F)F]

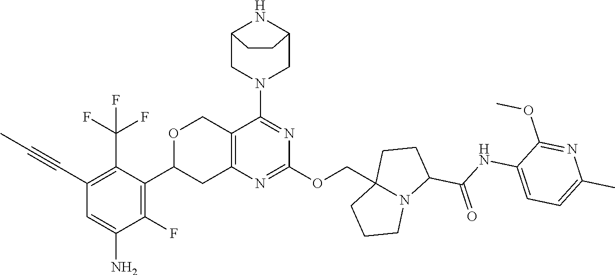 [SMILES: CC#Cc1cc(N)c(F)c(C2Cc3nc(OCC45CCCN4C(C(=O)Nc4ccc(C)nc4OC)CC5)nc(N4CC5CCC(C4)N5)c3CO2)c1C(F)(F)F]

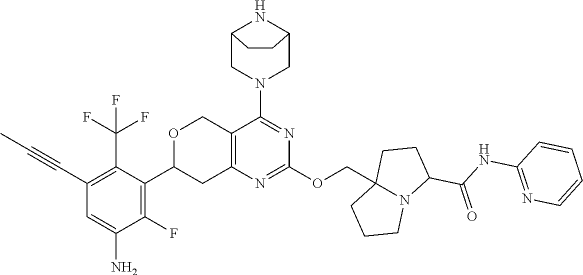 [SMILES: CC#Cc1cc(N)c(F)c(C2Cc3nc(OCC45CCCN4C(C(=O)Nc4ccccn4)CC5)nc(N4CC5CCC(C4)N5)c3CO2)c1C(F)(F)F]

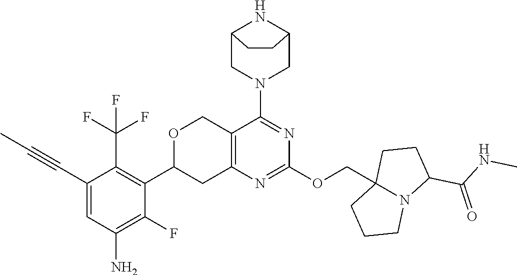 [SMILES: CC#Cc1cc(N)c(F)c(C2Cc3nc(OCC45CCCN4C(C(=O)NC)CC5)nc(N4CC5CCC(C4)N5)c3CO2)c1C(F)(F)F]

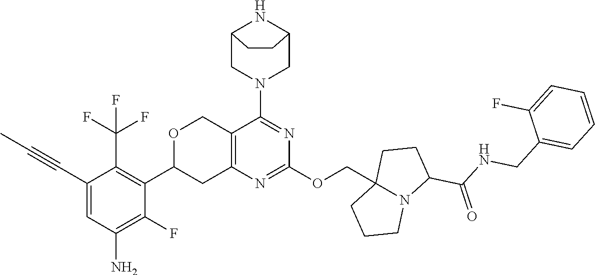 [SMILES: CC#Cc1cc(N)c(F)c(C2Cc3nc(OCC45CCCN4C(C(=O)NCc4ccccc4F)CC5)nc(N4CC5CCC(C4)N5)c3CO2)c1C(F)(F)F]

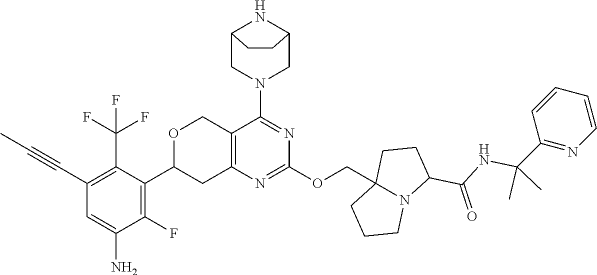 [SMILES: CC#Cc1cc(N)c(F)c(C2Cc3nc(OCC45CCCN4C(C(=O)NC(C)(C)c4ccccn4)CC5)nc(N4CC5CCC(C4)N5)c3CO2)c1C(F)(F)F]

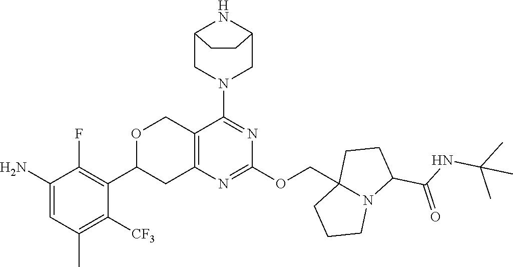 [SMILES: Cc1cc(N)c(F)c(C2Cc3nc(OCC45CCCN4C(C(=O)NC(C)(C)C)CC5)nc(N4CC5CCC(C4)N5)c3CO2)c1C(F)(F)F]

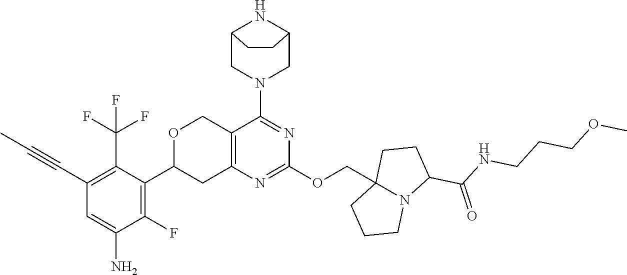 [SMILES: CC#Cc1cc(N)c(F)c(C2Cc3nc(OCC45CCCN4C(C(=O)NCCCOC)CC5)nc(N4CC5CCC(C4)N5)c3CO2)c1C(F)(F)F]

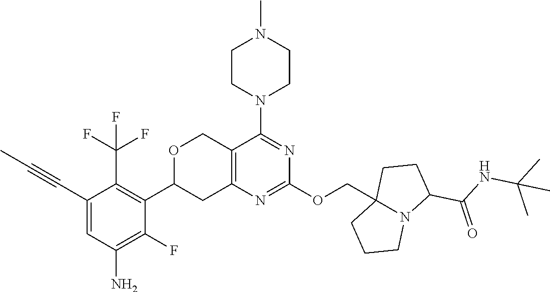 [SMILES: CC#Cc1cc(N)c(F)c(C2Cc3nc(OCC45CCCN4C(C(=O)NC(C)(C)C)CC5)nc(N4CCN(C)CC4)c3CO2)c1C(F)(F)F]

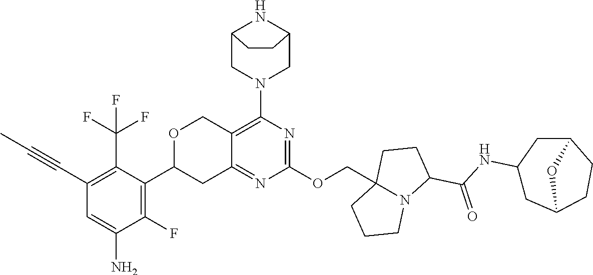 [SMILES: CC#Cc1cc(N)c(F)c(C2Cc3nc(OCC45CCCN4C(C(=O)NC4C[C@H]6CC[C@@H](C4)O6)CC5)nc(N4CC5CCC(C4)N5)c3CO2)c1C(F)(F)F]

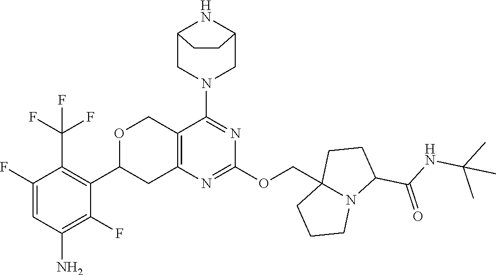 [SMILES: CC(C)(C)NC(=O)C1CCC2(COc3nc4c(c(N5CC6CCC(C5)N6)n3)COC(c3c(F)c(N)cc(F)c3C(F)(F)F)C4)CCCN12]

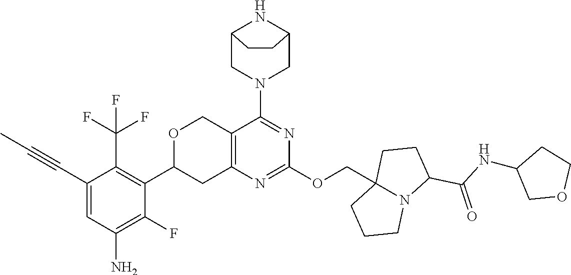 [SMILES: CC#Cc1cc(N)c(F)c(C2Cc3nc(OCC45CCCN4C(C(=O)NC4CCOC4)CC5)nc(N4CC5CCC(C4)N5)c3CO2)c1C(F)(F)F]

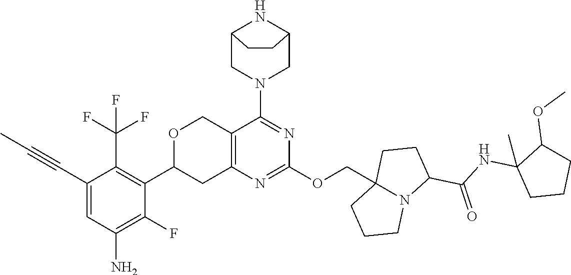 [SMILES: CC#Cc1cc(N)c(F)c(C2Cc3nc(OCC45CCCN4C(C(=O)NC4(C)CCCC4OC)CC5)nc(N4CC5CCC(C4)N5)c3CO2)c1C(F)(F)F]

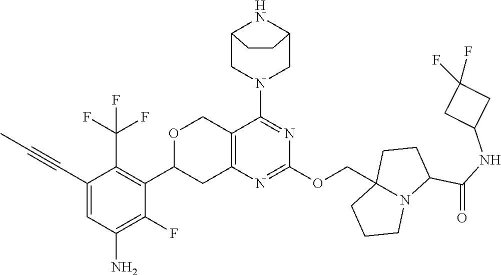 [SMILES: CC#Cc1cc(N)c(F)c(C2Cc3nc(OCC45CCCN4C(C(=O)NC4CC(F)(F)C4)CC5)nc(N4CC5CCC(C4)N5)c3CO2)c1C(F)(F)F]

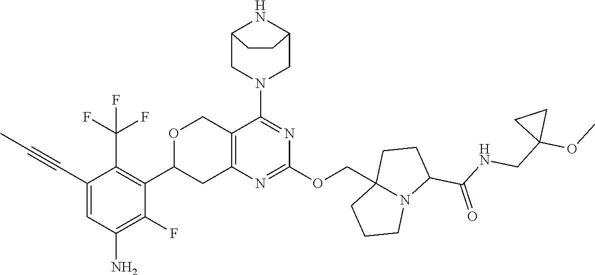 [SMILES: CC#Cc1cc(N)c(F)c(C2Cc3nc(OCC45CCCN4C(C(=O)NCC4(OC)CC4)CC5)nc(N4CC5CCC(C4)N5)c3CO2)c1C(F)(F)F]

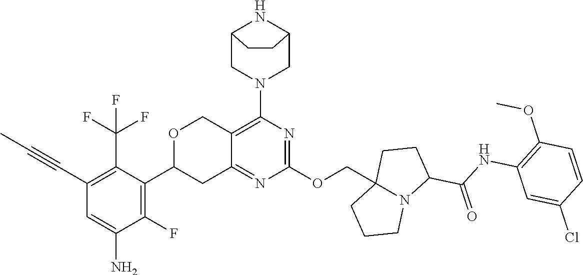 [SMILES: CC#Cc1cc(N)c(F)c(C2Cc3nc(OCC45CCCN4C(C(=O)Nc4cc(Cl)ccc4OC)CC5)nc(N4CC5CCC(C4)N5)c3CO2)c1C(F)(F)F]

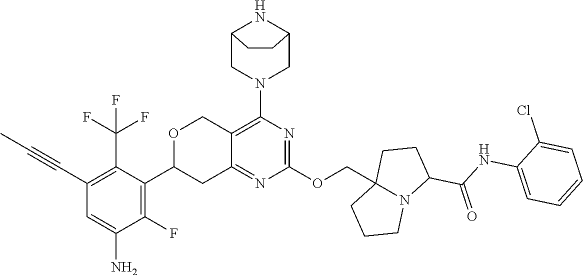 [SMILES: CC#Cc1cc(N)c(F)c(C2Cc3nc(OCC45CCCN4C(C(=O)Nc4ccccc4Cl)CC5)nc(N4CC5CCC(C4)N5)c3CO2)c1C(F)(F)F]

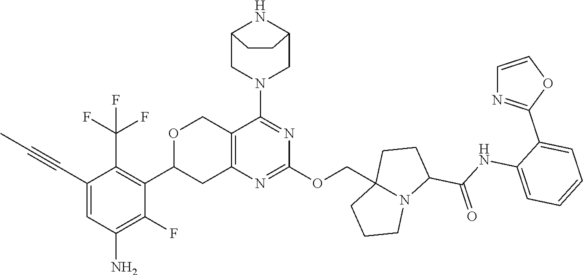 [SMILES: CC#Cc1cc(N)c(F)c(C2Cc3nc(OCC45CCCN4C(C(=O)Nc4ccccc4-c4ncco4)CC5)nc(N4CC5CCC(C4)N5)c3CO2)c1C(F)(F)F]

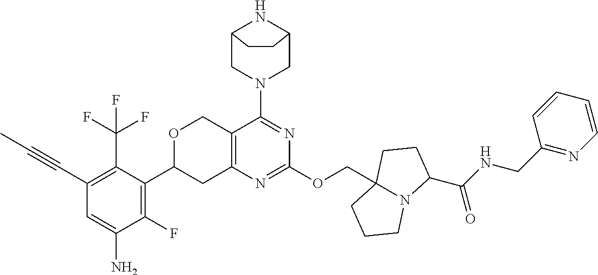 [SMILES: CC#Cc1cc(N)c(F)c(C2Cc3nc(OCC45CCCN4C(C(=O)NCc4ccccn4)CC5)nc(N4CC5CCC(C4)N5)c3CO2)c1C(F)(F)F]

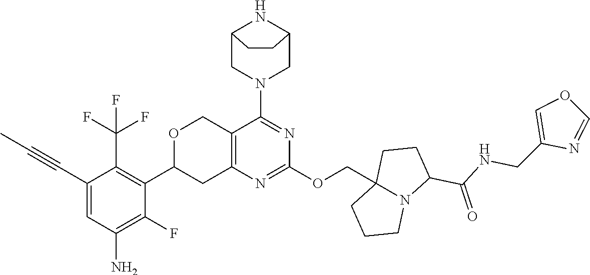 [SMILES: CC#Cc1cc(N)c(F)c(C2Cc3nc(OCC45CCCN4C(C(=O)NCc4cocn4)CC5)nc(N4CC5CCC(C4)N5)c3CO2)c1C(F)(F)F]

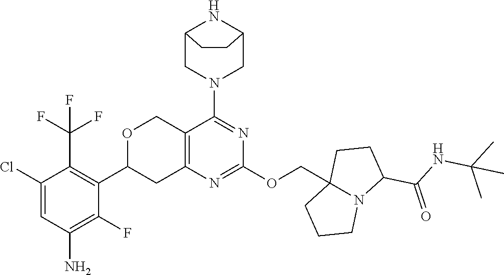 [SMILES: CC(C)(C)NC(=O)C1CCC2(COc3nc4c(c(N5CC6CCC(C5)N6)n3)COC(c3c(F)c(N)cc(Cl)c3C(F)(F)F)C4)CCCN12]

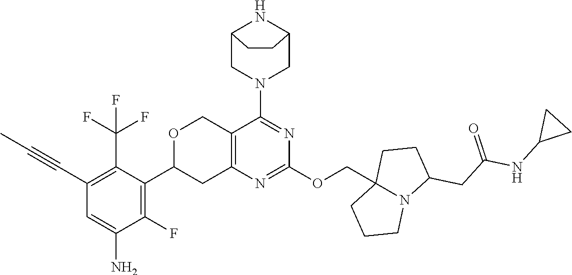 [SMILES: CC#Cc1cc(N)c(F)c(C2Cc3nc(OCC45CCCN4C(CC(=O)NC4CC4)CC5)nc(N4CC5CCC(C4)N5)c3CO2)c1C(F)(F)F]

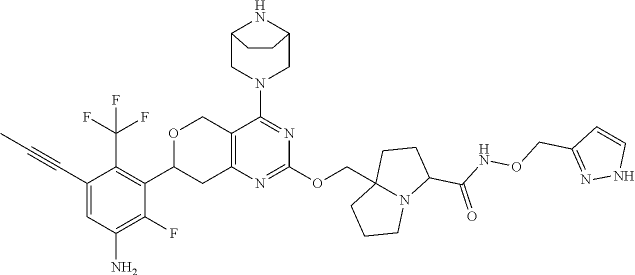 [SMILES: CC#Cc1cc(N)c(F)c(C2Cc3nc(OCC45CCCN4C(C(=O)NOCc4cc[nH]n4)CC5)nc(N4CC5CCC(C4)N5)c3CO2)c1C(F)(F)F]

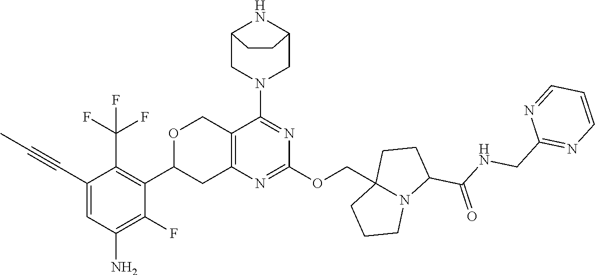 [SMILES: CC#Cc1cc(N)c(F)c(C2Cc3nc(OCC45CCCN4C(C(=O)NCc4ncccn4)CC5)nc(N4CC5CCC(C4)N5)c3CO2)c1C(F)(F)F]

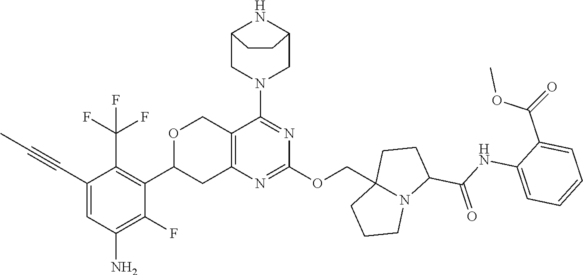 [SMILES: CC#Cc1cc(N)c(F)c(C2Cc3nc(OCC45CCCN4C(C(=O)Nc4ccccc4C(=O)OC)CC5)nc(N4CC5CCC(C4)N5)c3CO2)c1C(F)(F)F]

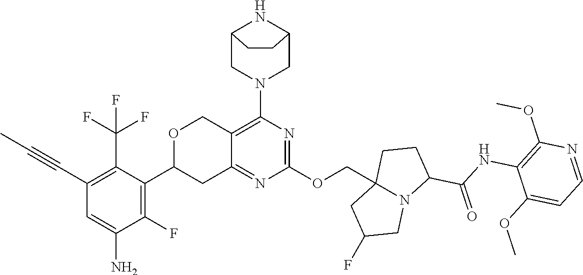 [SMILES: CC#Cc1cc(N)c(F)c(C2Cc3nc(OCC45CCC(C(=O)Nc6c(OC)ccnc6OC)N4CC(F)C5)nc(N4CC5CCC(C4)N5)c3CO2)c1C(F)(F)F]

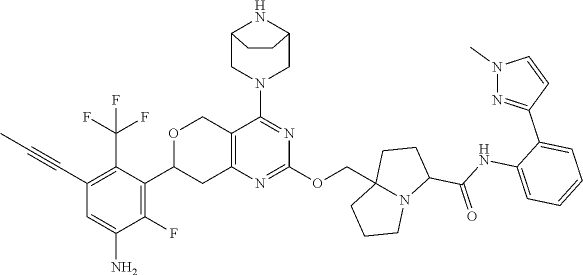 [SMILES: CC#Cc1cc(N)c(F)c(C2Cc3nc(OCC45CCCN4C(C(=O)Nc4ccccc4-c4ccn(C)n4)CC5)nc(N4CC5CCC(C4)N5)c3CO2)c1C(F)(F)F]

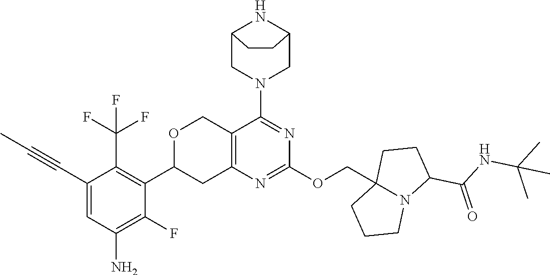 [SMILES: CC#Cc1cc(N)c(F)c(C2Cc3nc(OCC45CCCN4C(C(=O)NC(C)(C)C)CC5)nc(N4CC5CCC(C4)N5)c3CO2)c1C(F)(F)F]

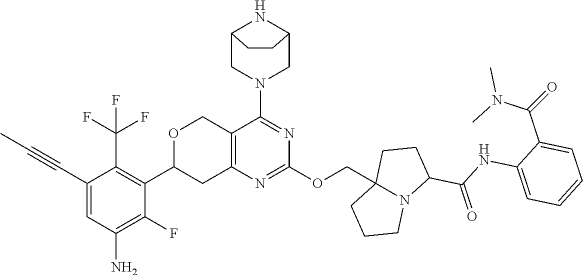 [SMILES: CC#Cc1cc(N)c(F)c(C2Cc3nc(OCC45CCCN4C(C(=O)Nc4ccccc4C(=O)N(C)C)CC5)nc(N4CC5CCC(C4)N5)c3CO2)c1C(F)(F)F]

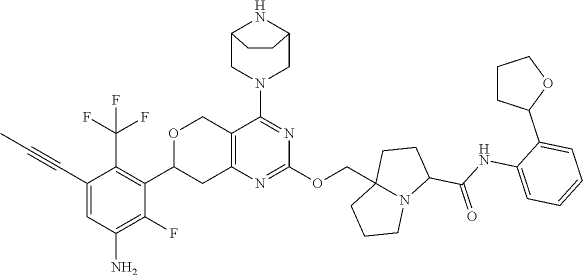 [SMILES: CC#Cc1cc(N)c(F)c(C2Cc3nc(OCC45CCCN4C(C(=O)Nc4ccccc4C4CCCO4)CC5)nc(N4CC5CCC(C4)N5)c3CO2)c1C(F)(F)F]

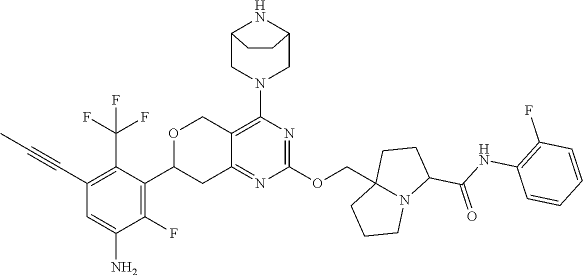 [SMILES: CC#Cc1cc(N)c(F)c(C2Cc3nc(OCC45CCCN4C(C(=O)Nc4ccccc4F)CC5)nc(N4CC5CCC(C4)N5)c3CO2)c1C(F)(F)F]

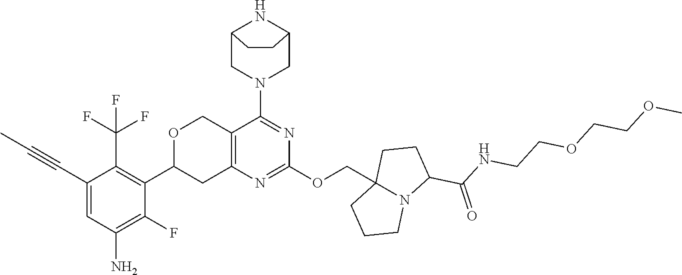 [SMILES: CC#Cc1cc(N)c(F)c(C2Cc3nc(OCC45CCCN4C(C(=O)NCCOCCOC)CC5)nc(N4CC5CCC(C4)N5)c3CO2)c1C(F)(F)F]

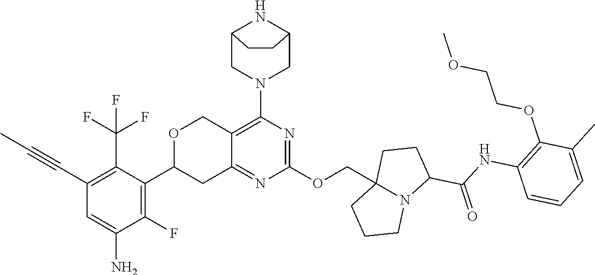 [SMILES: CC#Cc1cc(N)c(F)c(C2Cc3nc(OCC45CCCN4C(C(=O)Nc4cccc(C)c4OCCOC)CC5)nc(N4CC5CCC(C4)N5)c3CO2)c1C(F)(F)F]

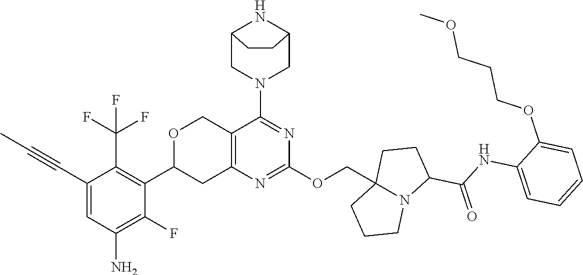 [SMILES: CC#Cc1cc(N)c(F)c(C2Cc3nc(OCC45CCCN4C(C(=O)Nc4ccccc4OCCCOC)CC5)nc(N4CC5CCC(C4)N5)c3CO2)c1C(F)(F)F]

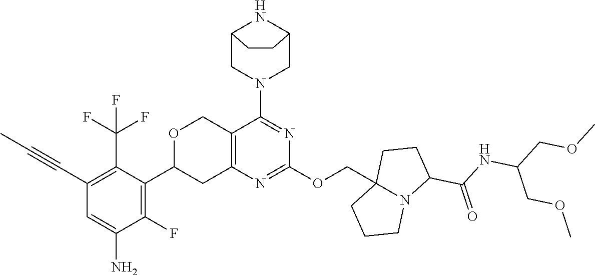 [SMILES: CC#Cc1cc(N)c(F)c(C2Cc3nc(OCC45CCCN4C(C(=O)NC(COC)COC)CC5)nc(N4CC5CCC(C4)N5)c3CO2)c1C(F)(F)F]

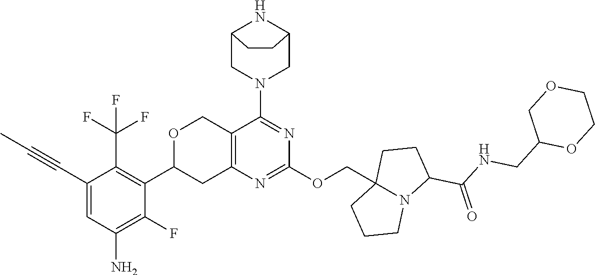 [SMILES: CC#Cc1cc(N)c(F)c(C2Cc3nc(OCC45CCCN4C(C(=O)NCC4COCCO4)CC5)nc(N4CC5CCC(C4)N5)c3CO2)c1C(F)(F)F]

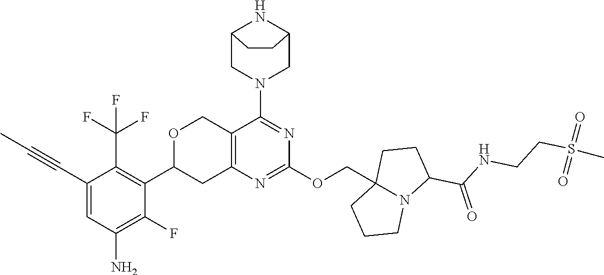 [SMILES: CC#Cc1cc(N)c(F)c(C2Cc3nc(OCC45CCCN4C(C(=O)NCCS(C)(=O)=O)CC5)nc(N4CC5CCC(C4)N5)c3CO2)c1C(F)(F)F]

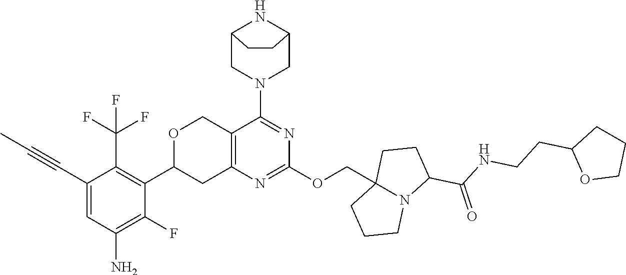 [SMILES: CC#Cc1cc(N)c(F)c(C2Cc3nc(OCC45CCCN4C(C(=O)NCCC4CCCO4)CC5)nc(N4CC5CCC(C4)N5)c3CO2)c1C(F)(F)F]

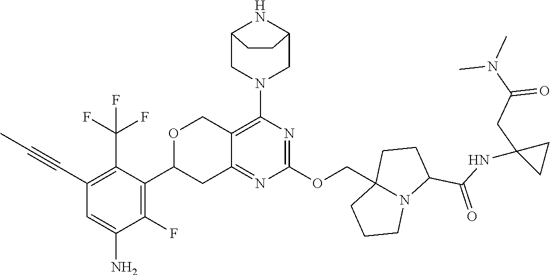 [SMILES: CC#Cc1cc(N)c(F)c(C2Cc3nc(OCC45CCCN4C(C(=O)NC4(CC(=O)N(C)C)CC4)CC5)nc(N4CC5CCC(C4)N5)c3CO2)c1C(F)(F)F]

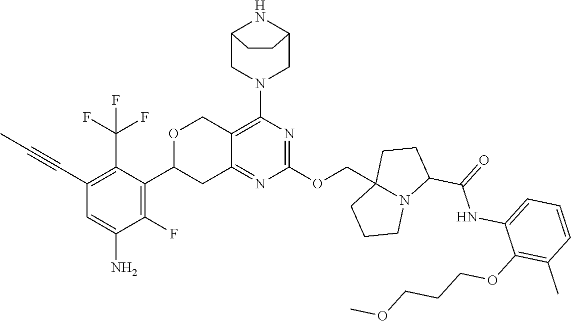 [SMILES: CC#Cc1cc(N)c(F)c(C2Cc3nc(OCC45CCCN4C(C(=O)Nc4cccc(C)c4OCCCOC)CC5)nc(N4CC5CCC(C4)N5)c3CO2)c1C(F)(F)F]

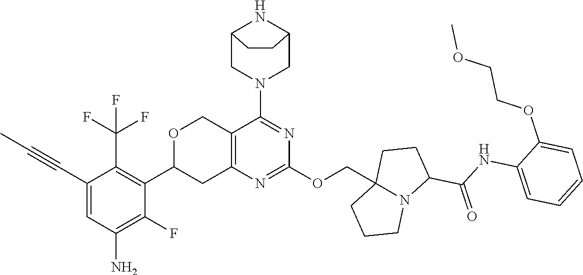 [SMILES: CC#Cc1cc(N)c(F)c(C2Cc3nc(OCC45CCCN4C(C(=O)Nc4ccccc4OCCOC)CC5)nc(N4CC5CCC(C4)N5)c3CO2)c1C(F)(F)F]

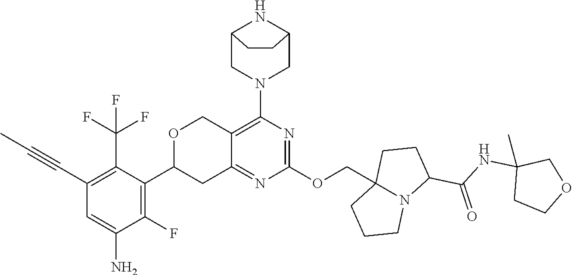 [SMILES: CC#Cc1cc(N)c(F)c(C2Cc3nc(OCC45CCCN4C(C(=O)NC4(C)CCOC4)CC5)nc(N4CC5CCC(C4)N5)c3CO2)c1C(F)(F)F]

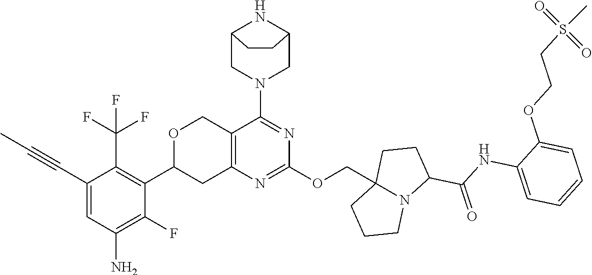 [SMILES: CC#Cc1cc(N)c(F)c(C2Cc3nc(OCC45CCCN4C(C(=O)Nc4ccccc4OCCS(C)(=O)=O)CC5)nc(N4CC5CCC(C4)N5)c3CO2)c1C(F)(F)F]

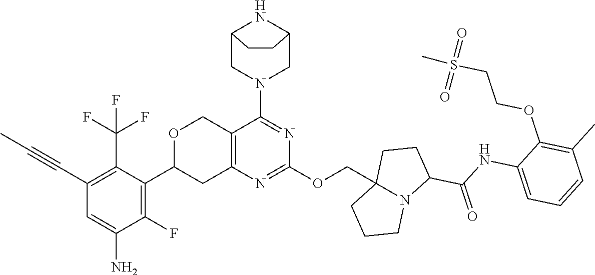 [SMILES: CC#Cc1cc(N)c(F)c(C2Cc3nc(OCC45CCCN4C(C(=O)Nc4cccc(C)c4OCCS(C)(=O)=O)CC5)nc(N4CC5CCC(C4)N5)c3CO2)c1C(F)(F)F]

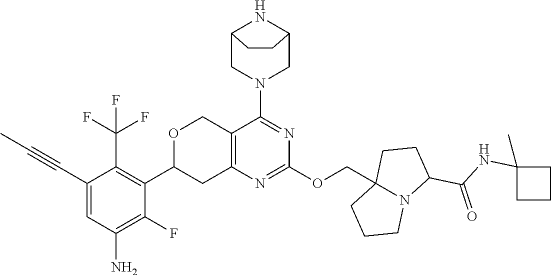 [SMILES: CC#Cc1cc(N)c(F)c(C2Cc3nc(OCC45CCCN4C(C(=O)NC4(C)CCC4)CC5)nc(N4CC5CCC(C4)N5)c3CO2)c1C(F)(F)F]

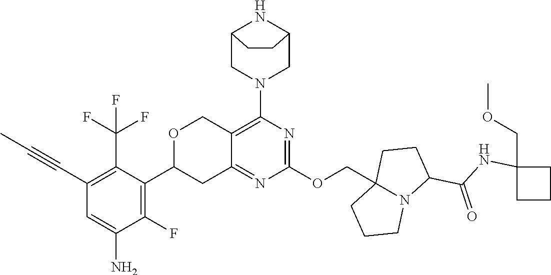 [SMILES: CC#Cc1cc(N)c(F)c(C2Cc3nc(OCC45CCCN4C(C(=O)NC4(COC)CCC4)CC5)nc(N4CC5CCC(C4)N5)c3CO2)c1C(F)(F)F]

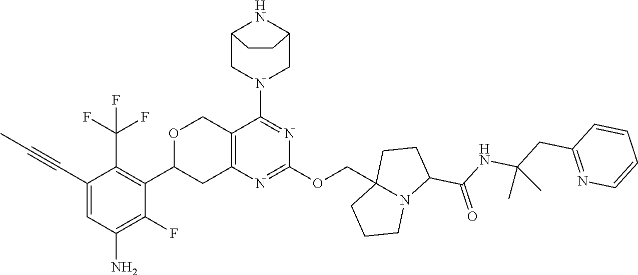 [SMILES: CC#Cc1cc(N)c(F)c(C2Cc3nc(OCC45CCCN4C(C(=O)NC(C)(C)Cc4ccccn4)CC5)nc(N4CC5CCC(C4)N5)c3CO2)c1C(F)(F)F]

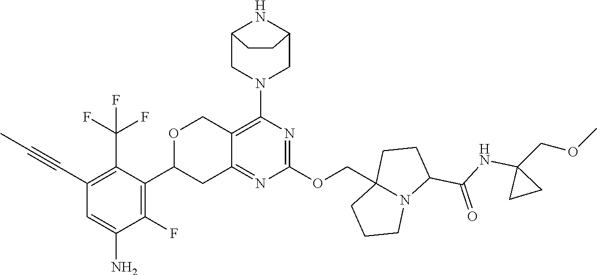 [SMILES: CC#Cc1cc(N)c(F)c(C2Cc3nc(OCC45CCCN4C(C(=O)NC4(COC)CC4)CC5)nc(N4CC5CCC(C4)N5)c3CO2)c1C(F)(F)F]

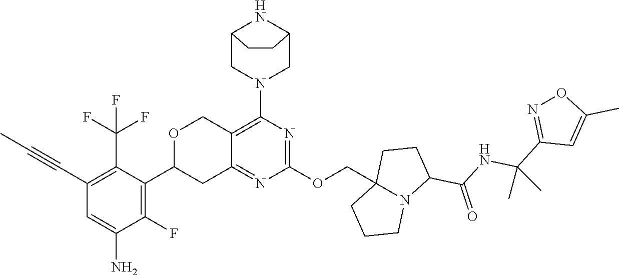 [SMILES: CC#Cc1cc(N)c(F)c(C2Cc3nc(OCC45CCCN4C(C(=O)NC(C)(C)c4cc(C)on4)CC5)nc(N4CC5CCC(C4)N5)c3CO2)c1C(F)(F)F]

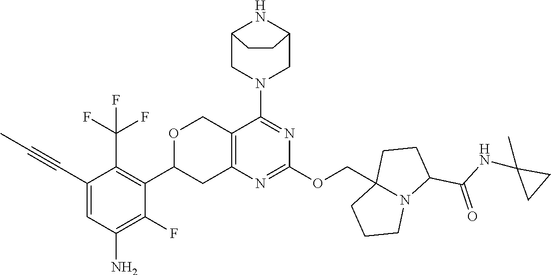 [SMILES: CC#Cc1cc(N)c(F)c(C2Cc3nc(OCC45CCCN4C(C(=O)NC4(C)CC4)CC5)nc(N4CC5CCC(C4)N5)c3CO2)c1C(F)(F)F]

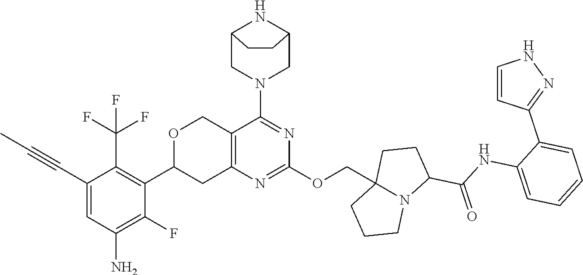 [SMILES: CC#Cc1cc(N)c(F)c(C2Cc3nc(OCC45CCCN4C(C(=O)Nc4ccccc4-c4cc[nH]n4)CC5)nc(N4CC5CCC(C4)N5)c3CO2)c1C(F)(F)F]

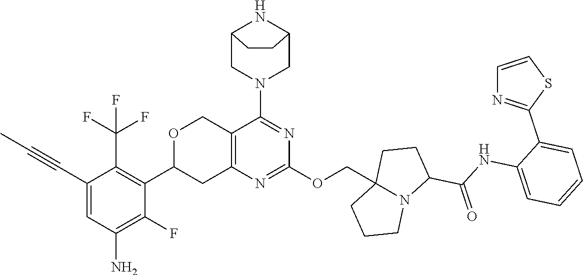 [SMILES: CC#Cc1cc(N)c(F)c(C2Cc3nc(OCC45CCCN4C(C(=O)Nc4ccccc4-c4nccs4)CC5)nc(N4CC5CCC(C4)N5)c3CO2)c1C(F)(F)F]

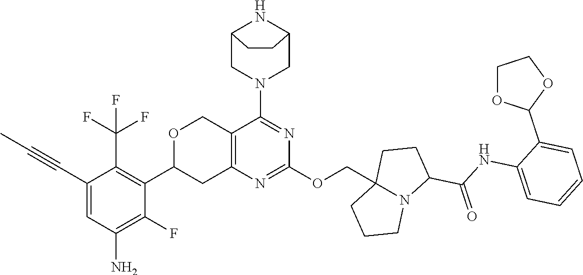 [SMILES: CC#Cc1cc(N)c(F)c(C2Cc3nc(OCC45CCCN4C(C(=O)Nc4ccccc4C4OCCO4)CC5)nc(N4CC5CCC(C4)N5)c3CO2)c1C(F)(F)F]

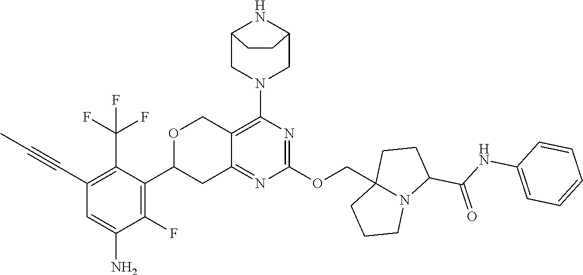 [SMILES: CC#Cc1cc(N)c(F)c(C2Cc3nc(OCC45CCCN4C(C(=O)Nc4ccccc4)CC5)nc(N4CC5CCC(C4)N5)c3CO2)c1C(F)(F)F]

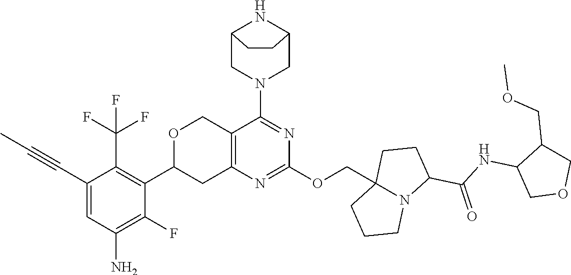 [SMILES: CC#Cc1cc(N)c(F)c(C2Cc3nc(OCC45CCCN4C(C(=O)NC4COCC4COC)CC5)nc(N4CC5CCC(C4)N5)c3CO2)c1C(F)(F)F]

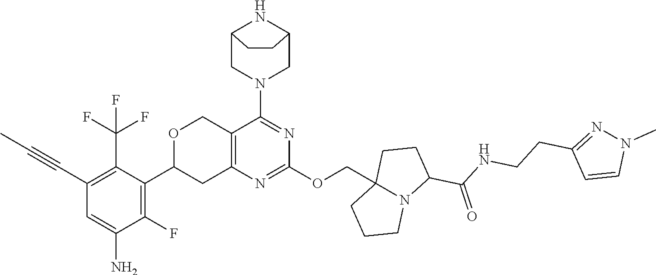 [SMILES: CC#Cc1cc(N)c(F)c(C2Cc3nc(OCC45CCCN4C(C(=O)NCCc4ccn(C)n4)CC5)nc(N4CC5CCC(C4)N5)c3CO2)c1C(F)(F)F]

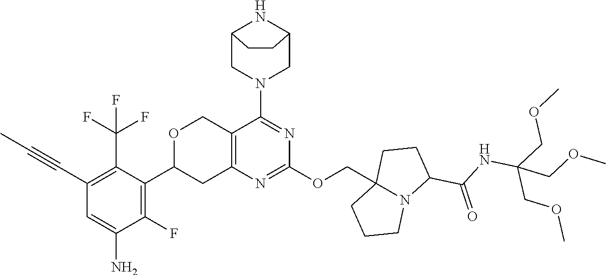 [SMILES: CC#Cc1cc(N)c(F)c(C2Cc3nc(OCC45CCCN4C(C(=O)NC(COC)(COC)COC)CC5)nc(N4CC5CCC(C4)N5)c3CO2)c1C(F)(F)F]

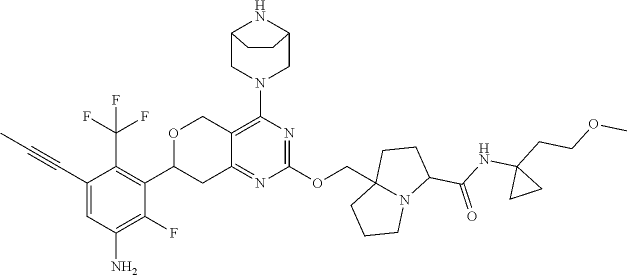 [SMILES: CC#Cc1cc(N)c(F)c(C2Cc3nc(OCC45CCCN4C(C(=O)NC4(CCOC)CC4)CC5)nc(N4CC5CCC(C4)N5)c3CO2)c1C(F)(F)F]

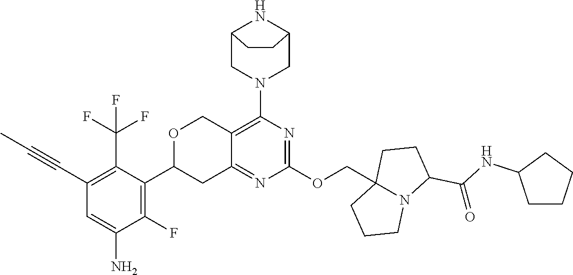 [SMILES: CC#Cc1cc(N)c(F)c(C2Cc3nc(OCC45CCCN4C(C(=O)NC4CCCC4)CC5)nc(N4CC5CCC(C4)N5)c3CO2)c1C(F)(F)F]

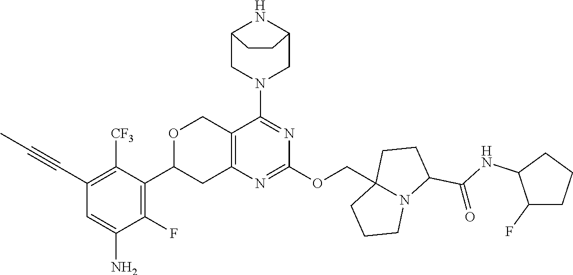 [SMILES: CC#Cc1cc(N)c(F)c(C2Cc3nc(OCC45CCCN4C(C(=O)NC4CCCC4F)CC5)nc(N4CC5CCC(C4)N5)c3CO2)c1C(F)(F)F]

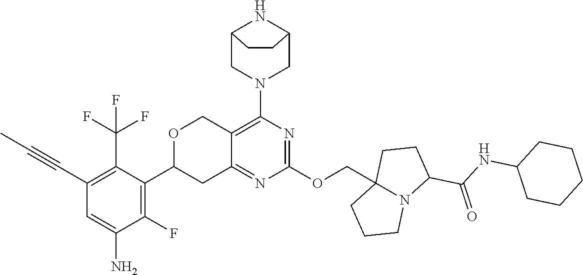 [SMILES: CC#Cc1cc(N)c(F)c(C2Cc3nc(OCC45CCCN4C(C(=O)NC4CCCCC4)CC5)nc(N4CC5CCC(C4)N5)c3CO2)c1C(F)(F)F]

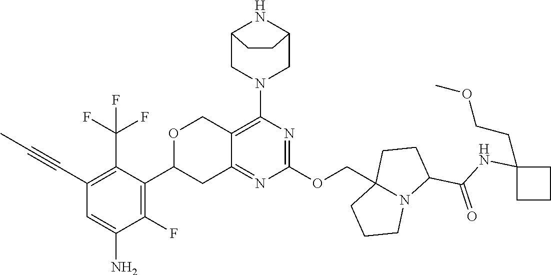 [SMILES: CC#Cc1cc(N)c(F)c(C2Cc3nc(OCC45CCCN4C(C(=O)NC4(CCOC)CCC4)CC5)nc(N4CC5CCC(C4)N5)c3CO2)c1C(F)(F)F]

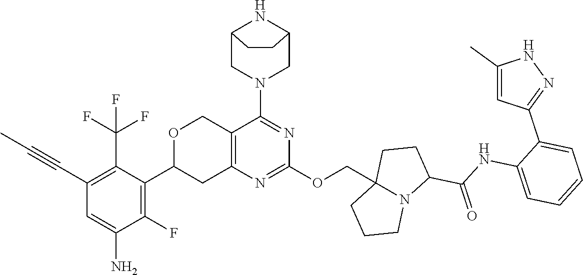 [SMILES: CC#Cc1cc(N)c(F)c(C2Cc3nc(OCC45CCCN4C(C(=O)Nc4ccccc4-c4cc(C)[nH]n4)CC5)nc(N4CC5CCC(C4)N5)c3CO2)c1C(F)(F)F]

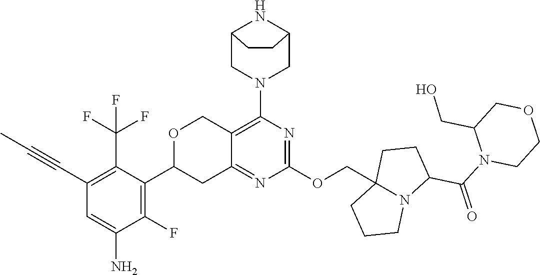 [SMILES: CC#Cc1cc(N)c(F)c(C2Cc3nc(OCC45CCCN4C(C(=O)N4CCOCC4CO)CC5)nc(N4CC5CCC(C4)N5)c3CO2)c1C(F)(F)F]

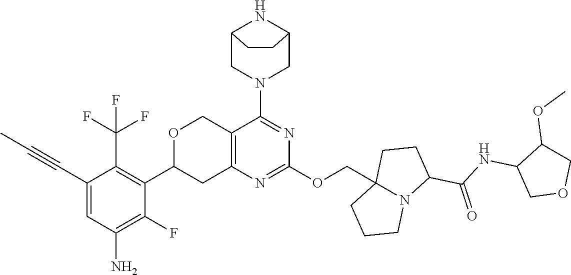 [SMILES: CC#Cc1cc(N)c(F)c(C2Cc3nc(OCC45CCCN4C(C(=O)NC4COCC4OC)CC5)nc(N4CC5CCC(C4)N5)c3CO2)c1C(F)(F)F]

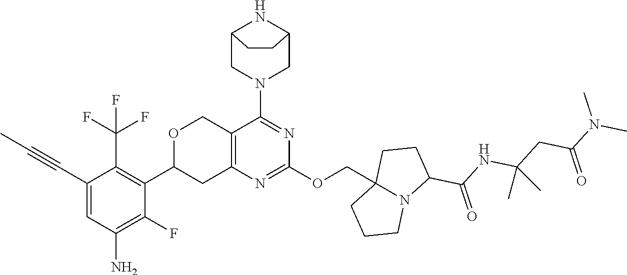 [SMILES: CC#Cc1cc(N)c(F)c(C2Cc3nc(OCC45CCCN4C(C(=O)NC(C)(C)CC(=O)N(C)C)CC5)nc(N4CC5CCC(C4)N5)c3CO2)c1C(F)(F)F]

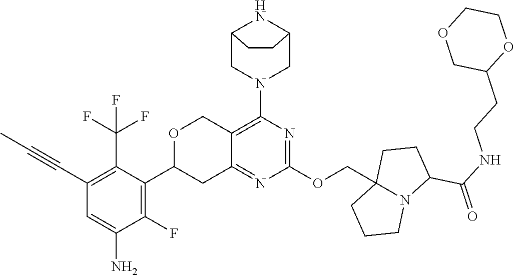 [SMILES: CC#Cc1cc(N)c(F)c(C2Cc3nc(OCC45CCCN4C(C(=O)NCCC4COCCO4)CC5)nc(N4CC5CCC(C4)N5)c3CO2)c1C(F)(F)F]

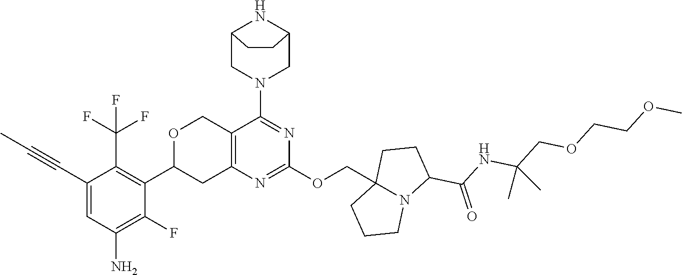 [SMILES: CC#Cc1cc(N)c(F)c(C2Cc3nc(OCC45CCCN4C(C(=O)NC(C)(C)COCCOC)CC5)nc(N4CC5CCC(C4)N5)c3CO2)c1C(F)(F)F]

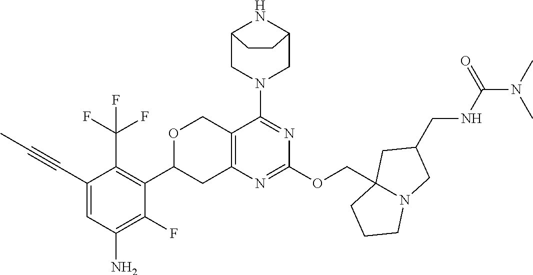 [SMILES: CC#Cc1cc(N)c(F)c(C2Cc3nc(OCC45CCCN4CC(CNC(=O)N(C)C)C5)nc(N4CC5CCC(C4)N5)c3CO2)c1C(F)(F)F]

TABLE 3

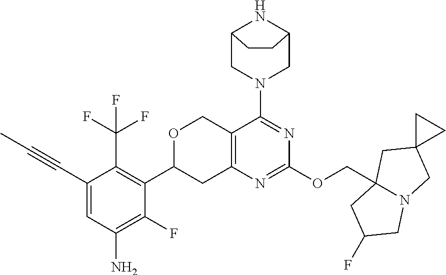 [SMILES: CC#Cc1cc(N)c(F)c(C2Cc3nc(OCC45CC(F)CN4CC4(CC4)C5)nc(N4CC5CCC(C4)N5)c3CO2)c1C(F)(F)F]

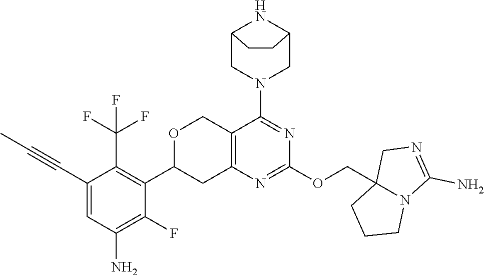 [SMILES: CC#Cc1cc(N)c(F)c(C2Cc3nc(OCC45CCCN4C(N)=NC5)nc(N4CC5CCC(C4)N5)c3CO2)c1C(F)(F)F]

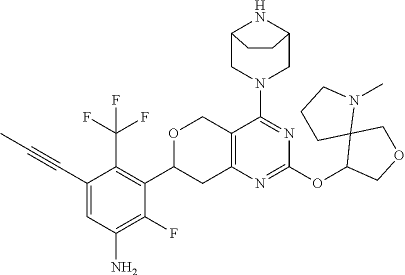 [SMILES: CC#Cc1cc(N)c(F)c(C2Cc3nc(OC4COCC45CCCN5C)nc(N4CC5CCC(C4)N5)c3CO2)c1C(F)(F)F]

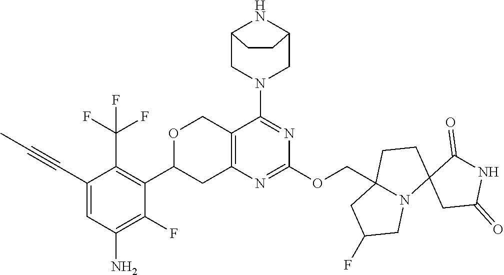 [SMILES: CC#Cc1cc(N)c(F)c(C2Cc3nc(OCC45CCC6(CC(=O)NC6=O)N4CC(F)C5)nc(N4CC5CCC(C4)N5)c3CO2)c1C(F)(F)F]

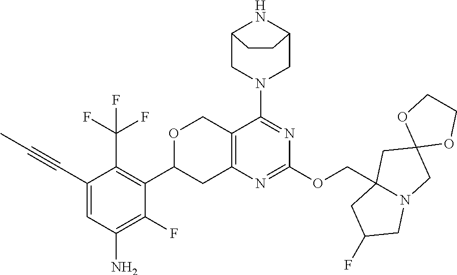 [SMILES: CC#Cc1cc(N)c(F)c(C2Cc3nc(OCC45CC(F)CN4CC4(C5)OCCO4)nc(N4CC5CCC(C4)N5)c3CO2)c1C(F)(F)F]

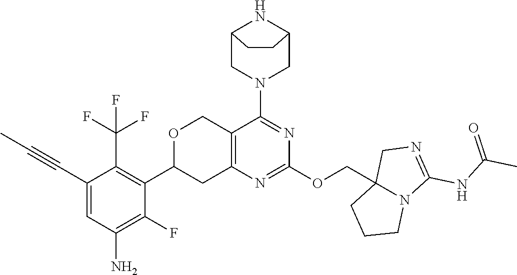 [SMILES: CC#Cc1cc(N)c(F)c(C2Cc3nc(OCC45CCCN4C(NC(C)=O)=NC5)nc(N4CC5CCC(C4)N5)c3CO2)c1C(F)(F)F]

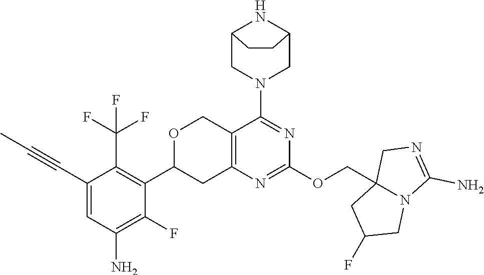 [SMILES: CC#Cc1cc(N)c(F)c(C2Cc3nc(OCC45CN=C(N)N4CC(F)C5)nc(N4CC5CCC(C4)N5)c3CO2)c1C(F)(F)F]

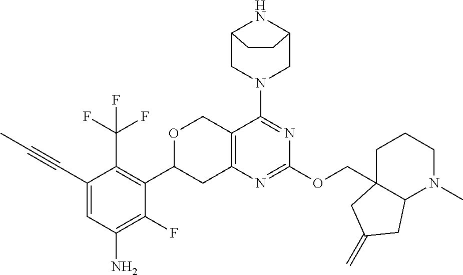 [SMILES: C=C1CC2N(C)CCCC2(COc2nc3c(c(N4CC5CCC(C4)N5)n2)COC(c2c(F)c(N)cc(C#CC)c2C(F)(F)F)C3)C1]

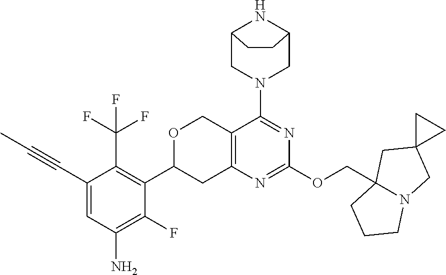 [SMILES: CC#Cc1cc(N)c(F)c(C2Cc3nc(OCC45CCCN4CC4(CC4)C5)nc(N4CC5CCC(C4)N5)c3CO2)c1C(F)(F)F]

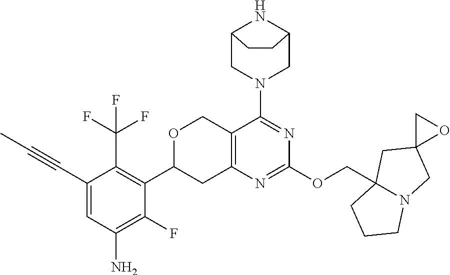 [SMILES: CC#Cc1cc(N)c(F)c(C2Cc3nc(OCC45CCCN4CC4(CO4)C5)nc(N4CC5CCC(C4)N5)c3CO2)c1C(F)(F)F]

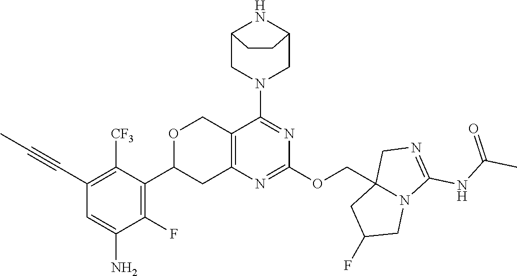 [SMILES: CC#Cc1cc(N)c(F)c(C2Cc3nc(OCC45CN=C(NC(C)=O)N4CC(F)C5)nc(N4CC5CCC(C4)N5)c3CO2)c1C(F)(F)F]

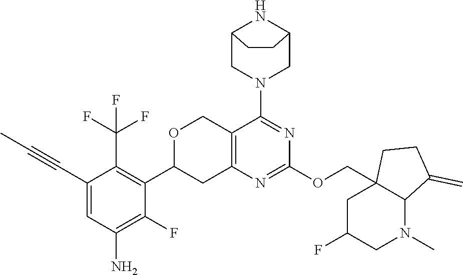 [SMILES: C=C1CCC2(COc3nc4c(c(N5CC6CCC(C5)N6)n3)COC(c3c(F)c(N)cc(C#CC)c3C(F)(F)F)C4)CC(F)CN(C)C12]

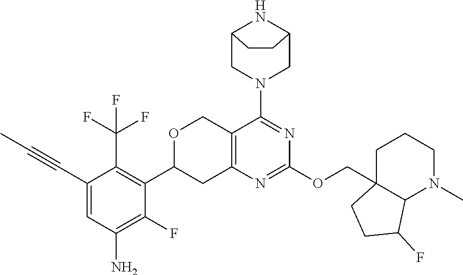 [SMILES: CC#Cc1cc(N)c(F)c(C2Cc3nc(OCC45CCCN(C)C4C(F)CC5)nc(N4CC5CCC(C4)N5)c3CO2)c1C(F)(F)F]

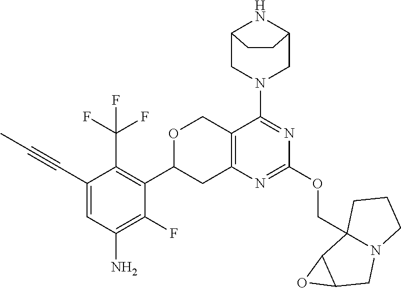 [SMILES: CC#Cc1cc(N)c(F)c(C2Cc3nc(OCC45CCCN4CC4OC45)nc(N4CC5CCC(C4)N5)c3CO2)c1C(F)(F)F]

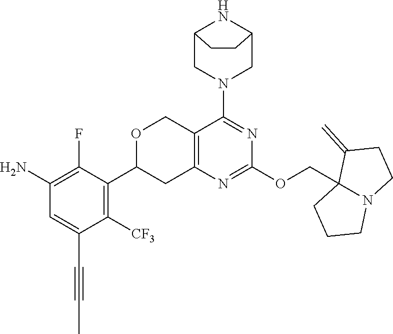 [SMILES: C=C1CCN2CCCC12COc1nc2c(c(N3CC4CCC(C3)N4)n1)COC(c1c(F)c(N)cc(C#CC)c1C(F)(F)F)C2]

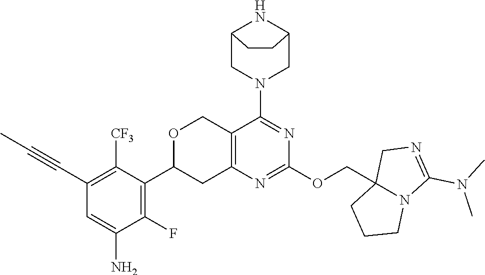 [SMILES: CC#Cc1cc(N)c(F)c(C2Cc3nc(OCC45CCCN4C(N(C)C)=NC5)nc(N4CC5CCC(C4)N5)c3CO2)c1C(F)(F)F]

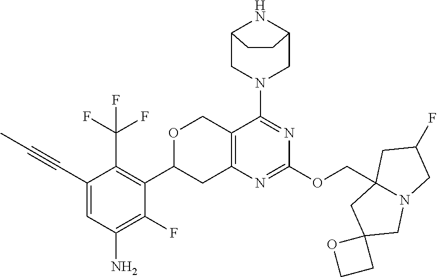 [SMILES: CC#Cc1cc(N)c(F)c(C2Cc3nc(OCC45CC(F)CN4CC4(CCO4)C5)nc(N4CC5CCC(C4)N5)c3CO2)c1C(F)(F)F]

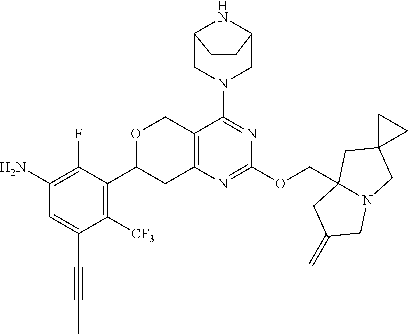 [SMILES: C=C1CN2CC3(CC3)CC2(COc2nc3c(c(N4CC5CCC(C4)N5)n2)COC(c2c(F)c(N)cc(C#CC)c2C(F)(F)F)C3)C1]

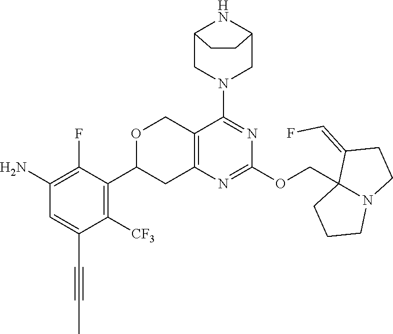 [SMILES: CC#Cc1cc(N)c(F)c(C2Cc3nc(OCC45CCCN4CC/C5=C/F)nc(N4CC5CCC(C4)N5)c3CO2)c1C(F)(F)F]

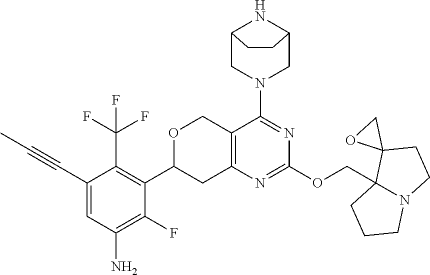 [SMILES: CC#Cc1cc(N)c(F)c(C2Cc3nc(OCC45CCCN4CCC54CO4)nc(N4CC5CCC(C4)N5)c3CO2)c1C(F)(F)F]

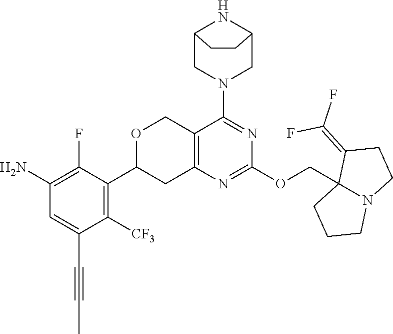 [SMILES: CC#Cc1cc(N)c(F)c(C2Cc3nc(OCC45CCCN4CCC5=C(F)F)nc(N4CC5CCC(C4)N5)c3CO2)c1C(F)(F)F]

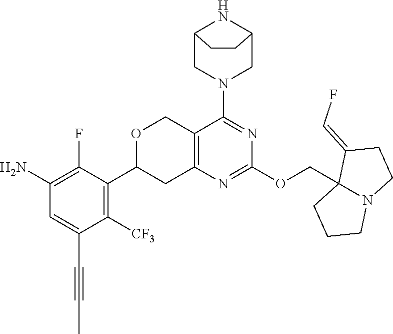 [SMILES: CC#Cc1cc(N)c(F)c(C2Cc3nc(OCC45CCCN4CC/C5=C\F)nc(N4CC5CCC(C4)N5)c3CO2)c1C(F)(F)F]

TABLE 4

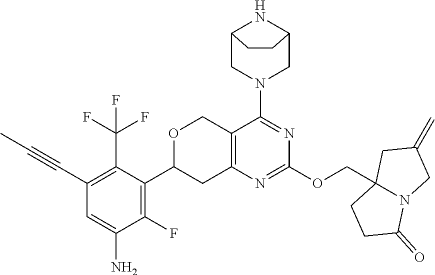 [SMILES: C=C1CN2C(=O)CCC2(COc2nc3c(c(N4CC5CCC(C4)N5)n2)COC(c2c(F)c(N)cc(C#CC)c2C(F)(F)F)C3)C1]

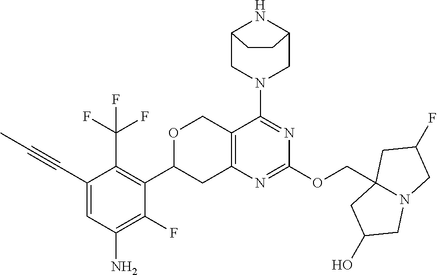 [SMILES: CC#Cc1cc(N)c(F)c(C2Cc3nc(OCC45CC(O)CN4CC(F)C5)nc(N4CC5CCC(C4)N5)c3CO2)c1C(F)(F)F]

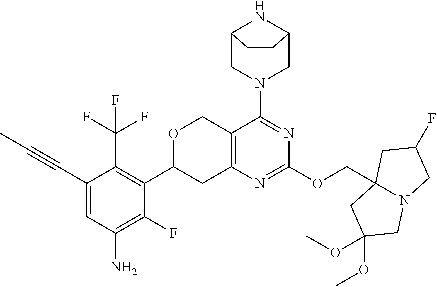 [SMILES: CC#Cc1cc(N)c(F)c(C2Cc3nc(OCC45CC(F)CN4CC(OC)(OC)C5)nc(N4CC5CCC(C4)N5)c3CO2)c1C(F)(F)F]

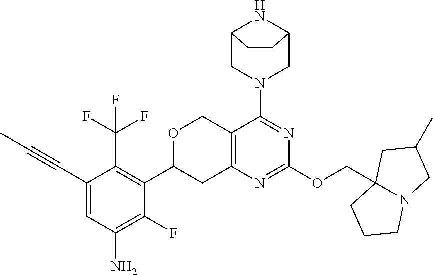 [SMILES: CC#Cc1cc(N)c(F)c(C2Cc3nc(OCC45CCCN4CC(C)C5)nc(N4CC5CCC(C4)N5)c3CO2)c1C(F)(F)F]

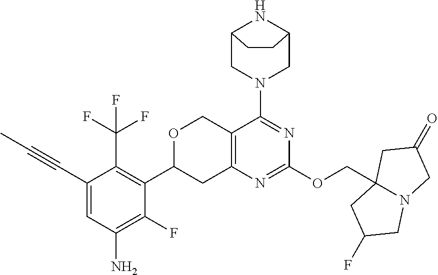 [SMILES: CC#Cc1cc(N)c(F)c(C2Cc3nc(OCC45CC(=O)CN4CC(F)C5)nc(N4CC5CCC(C4)N5)c3CO2)c1C(F)(F)F]

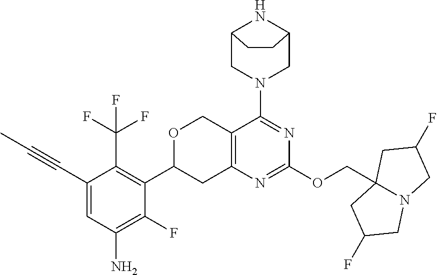 [SMILES: CC#Cc1cc(N)c(F)c(C2Cc3nc(OCC45CC(F)CN4CC(F)C5)nc(N4CC5CCC(C4)N5)c3CO2)c1C(F)(F)F]

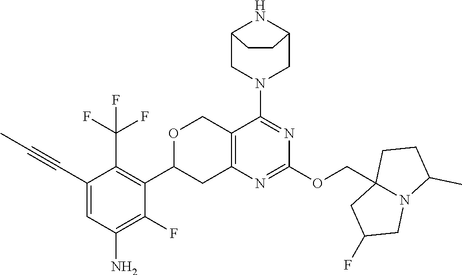 [SMILES: CC#Cc1cc(N)c(F)c(C2Cc3nc(OCC45CCC(C)N4CC(F)C5)nc(N4CC5CCC(C4)N5)c3CO2)c1C(F)(F)F]

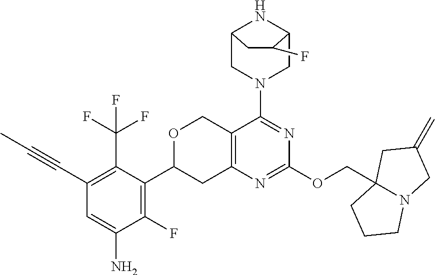 [SMILES: C=C1CN2CCCC2(COc2nc3c(c(N4CC5CC(F)C(C4)N5)n2)COC(c2c(F)c(N)cc(C#CC)c2C(F)(F)F)C3)C1]

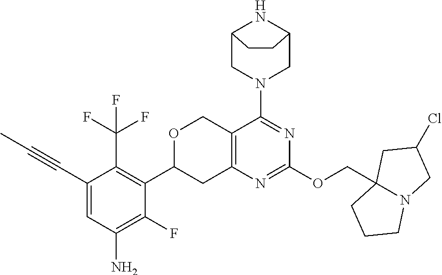 [SMILES: CC#Cc1cc(N)c(F)c(C2Cc3nc(OCC45CCCN4CC(Cl)C5)nc(N4CC5CCC(C4)N5)c3CO2)c1C(F)(F)F]

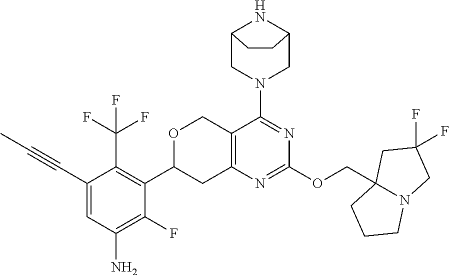 [SMILES: CC#Cc1cc(N)c(F)c(C2Cc3nc(OCC45CCCN4CC(F)(F)C5)nc(N4CC5CCC(C4)N5)c3CO2)c1C(F)(F)F]

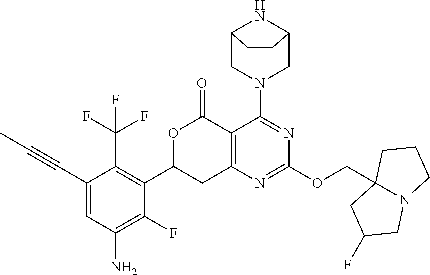 [SMILES: CC#Cc1cc(N)c(F)c(C2Cc3nc(OCC45CCCN4CC(F)C5)nc(N4CC5CCC(C4)N5)c3C(=O)O2)c1C(F)(F)F]

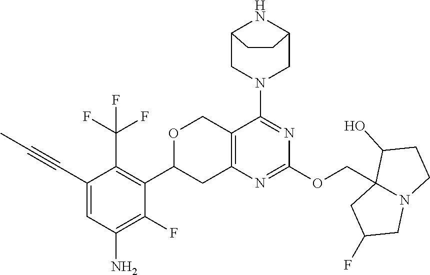 [SMILES: CC#Cc1cc(N)c(F)c(C2Cc3nc(OCC45CC(F)CN4CCC5O)nc(N4CC5CCC(C4)N5)c3CO2)c1C(F)(F)F]

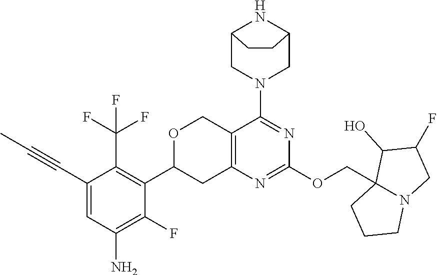 [SMILES: CC#Cc1cc(N)c(F)c(C2Cc3nc(OCC45CCCN4CC(F)C5O)nc(N4CC5CCC(C4)N5)c3CO2)c1C(F)(F)F]

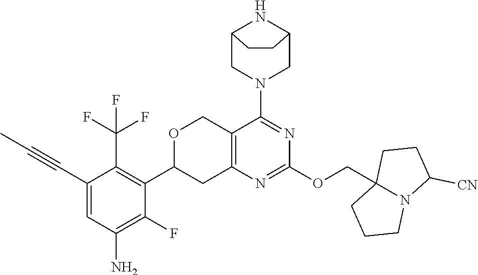 [SMILES: CC#Cc1cc(N)c(F)c(C2Cc3nc(OCC45CCCN4C(C#N)CC5)nc(N4CC5CCC(C4)N5)c3CO2)c1C(F)(F)F]

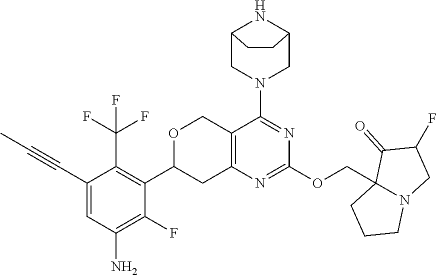 [SMILES: CC#Cc1cc(N)c(F)c(C2Cc3nc(OCC45CCCN4CC(F)C5=O)nc(N4CC5CCC(C4)N5)c3CO2)c1C(F)(F)F]

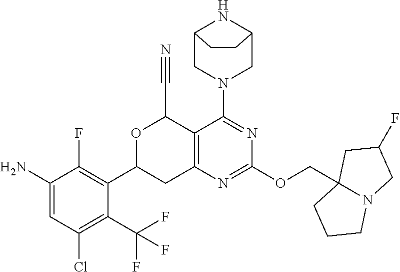 [SMILES: N#CC1OC(c2c(F)c(N)cc(Cl)c2C(F)(F)F)Cc2nc(OCC34CCCN3CC(F)C4)nc(N3CC4CCC(C3)N4)c21]

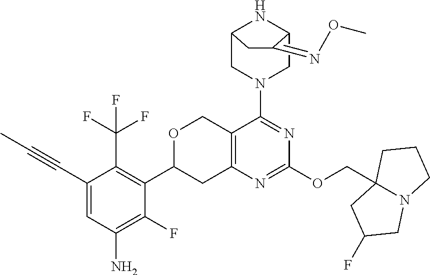 [SMILES: CC#Cc1cc(N)c(F)c(C2Cc3nc(OCC45CCCN4CC(F)C5)nc(N4CC5C/C(=N/OC)C(C4)N5)c3CO2)c1C(F)(F)F]

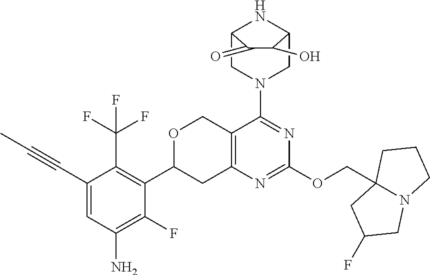 [SMILES: CC#Cc1cc(N)c(F)c(C2Cc3nc(OCC45CCCN4CC(F)C5)nc(N4CC5NC(C4)C(O)C5=O)c3CO2)c1C(F)(F)F]

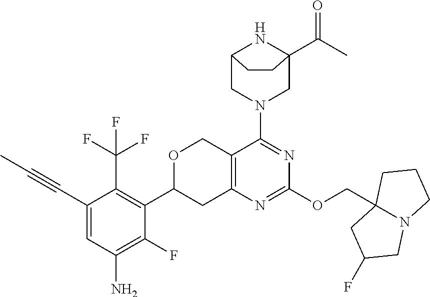 [SMILES: CC#Cc1cc(N)c(F)c(C2Cc3nc(OCC45CCCN4CC(F)C5)nc(N4CC5CCC(C(C)=O)(C4)N5)c3CO2)c1C(F)(F)F]

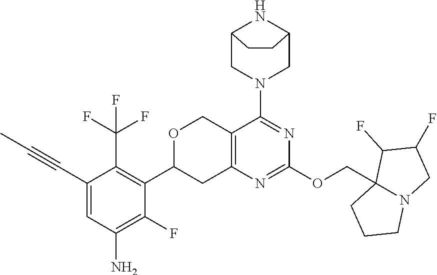 [SMILES: CC#Cc1cc(N)c(F)c(C2Cc3nc(OCC45CCCN4CC(F)C5F)nc(N4CC5CCC(C4)N5)c3CO2)c1C(F)(F)F]

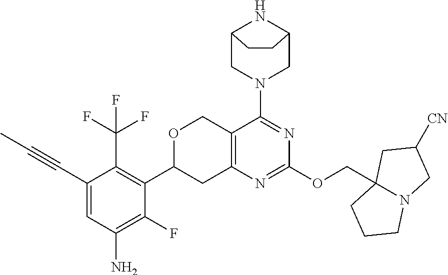 [SMILES: CC#Cc1cc(N)c(F)c(C2Cc3nc(OCC45CCCN4CC(C#N)C5)nc(N4CC5CCC(C4)N5)c3CO2)c1C(F)(F)F]

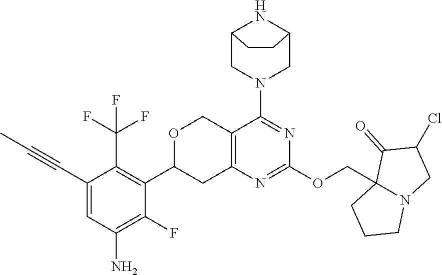 [SMILES: CC#Cc1cc(N)c(F)c(C2Cc3nc(OCC45CCCN4CC(Cl)C5=O)nc(N4CC5CCC(C4)N5)c3CO2)c1C(F)(F)F]

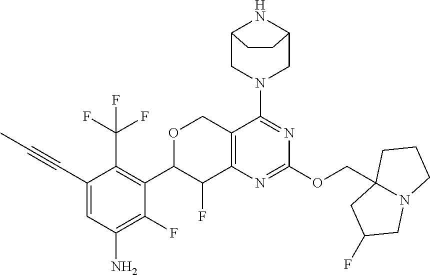 [SMILES: CC#Cc1cc(N)c(F)c(C2OCc3c(nc(OCC45CCCN4CC(F)C5)nc3N3CC4CCC(C3)N4)C2F)c1C(F)(F)F]

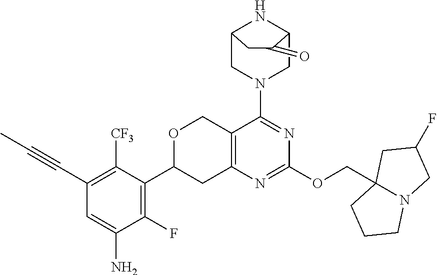 [SMILES: CC#Cc1cc(N)c(F)c(C2Cc3nc(OCC45CCCN4CC(F)C5)nc(N4CC5CC(=O)C(C4)N5)c3CO2)c1C(F)(F)F]

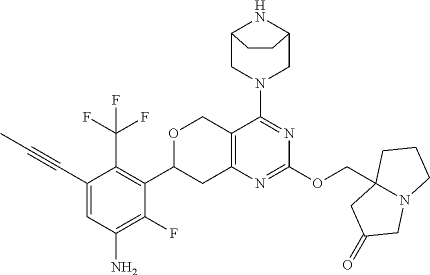 [SMILES: CC#Cc1cc(N)c(F)c(C2Cc3nc(OCC45CCCN4CC(=O)C5)nc(N4CC5CCC(C4)N5)c3CO2)c1C(F)(F)F]

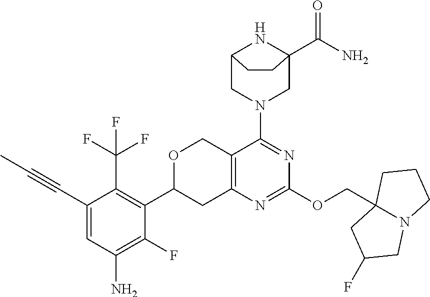 [SMILES: CC#Cc1cc(N)c(F)c(C2Cc3nc(OCC45CCCN4CC(F)C5)nc(N4CC5CCC(C(N)=O)(C4)N5)c3CO2)c1C(F)(F)F]

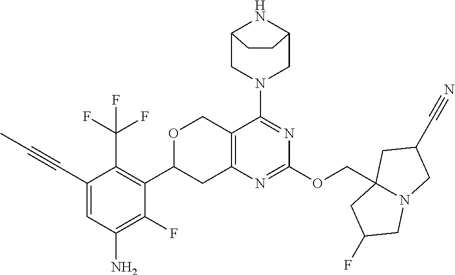 [SMILES: CC#Cc1cc(N)c(F)c(C2Cc3nc(OCC45CC(F)CN4CC(C#N)C5)nc(N4CC5CCC(C4)N5)c3CO2)c1C(F)(F)F]

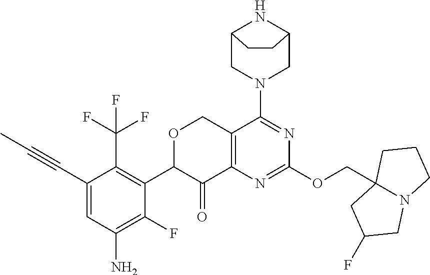 [SMILES: CC#Cc1cc(N)c(F)c(C2OCc3c(nc(OCC45CCCN4CC(F)C5)nc3N3CC4CCC(C3)N4)C2=O)c1C(F)(F)F]

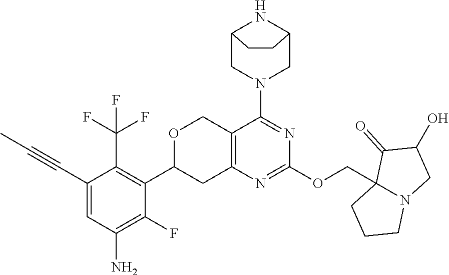 [SMILES: CC#Cc1cc(N)c(F)c(C2Cc3nc(OCC45CCCN4CC(O)C5=O)nc(N4CC5CCC(C4)N5)c3CO2)c1C(F)(F)F]

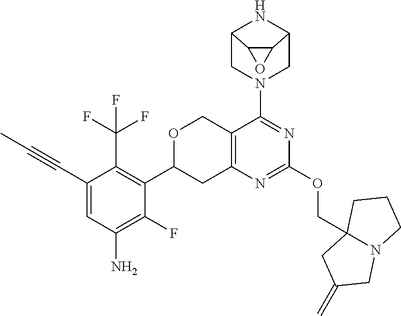 [SMILES: C=C1CN2CCCC2(COc2nc3c(c(N4CC5NC(C4)C4OC54)n2)COC(c2c(F)c(N)cc(C#CC)c2C(F)(F)F)C3)C1]

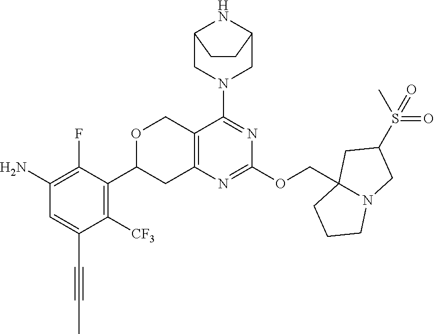 [SMILES: CC#Cc1cc(N)c(F)c(C2Cc3nc(OCC45CCCN4CC(S(C)(=O)=O)C5)nc(N4CC5CCC(C4)N5)c3CO2)c1C(F)(F)F]

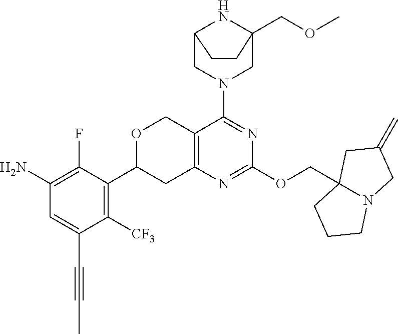 [SMILES: C=C1CN2CCCC2(COc2nc3c(c(N4CC5CCC(COC)(C4)N5)n2)COC(c2c(F)c(N)cc(C#CC)c2C(F)(F)F)C3)C1]

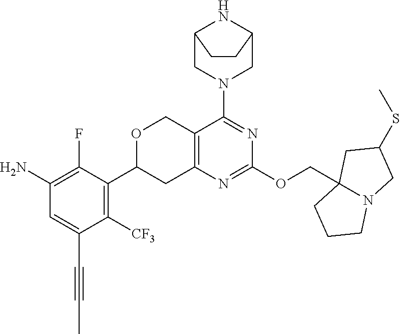 [SMILES: CC#Cc1cc(N)c(F)c(C2Cc3nc(OCC45CCCN4CC(SC)C5)nc(N4CC5CCC(C4)N5)c3CO2)c1C(F)(F)F]

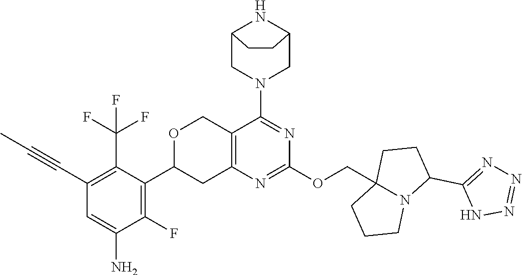 [SMILES: CC#Cc1cc(N)c(F)c(C2Cc3nc(OCC45CCCN4C(c4nnn[nH]4)CC5)nc(N4CC5CCC(C4)N5)c3CO2)c1C(F)(F)F]

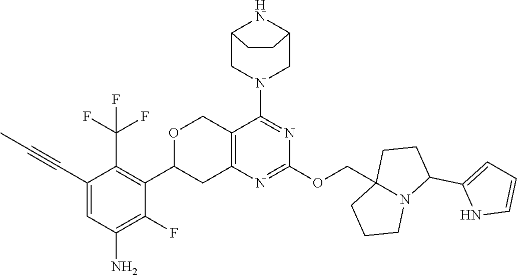 [SMILES: CC#Cc1cc(N)c(F)c(C2Cc3nc(OCC45CCCN4C(c4ccc[nH]4)CC5)nc(N4CC5CCC(C4)N5)c3CO2)c1C(F)(F)F]

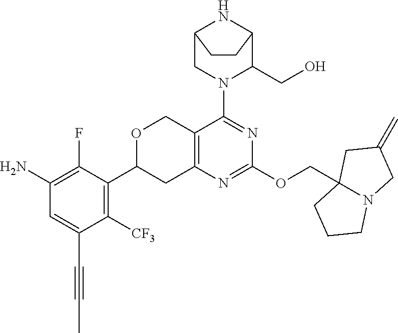 [SMILES: C=C1CN2CCCC2(COc2nc3c(c(N4CC5CCC(N5)C4CO)n2)COC(c2c(F)c(N)cc(C#CC)c2C(F)(F)F)C3)C1]

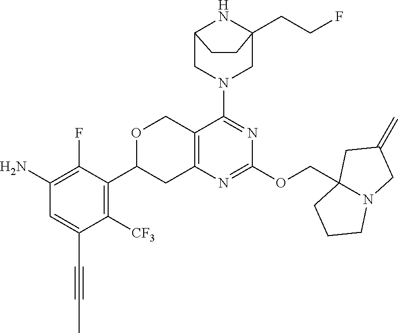 [SMILES: C=C1CN2CCCC2(COc2nc3c(c(N4CC5CCC(CCF)(C4)N5)n2)COC(c2c(F)c(N)cc(C#CC)c2C(F)(F)F)C3)C1]

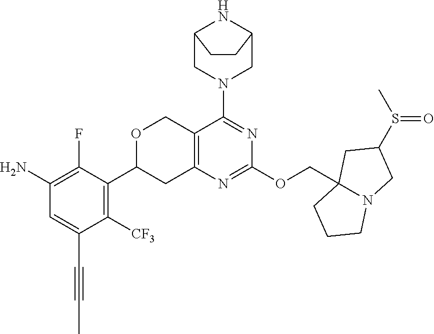 [SMILES: CC#Cc1cc(N)c(F)c(C2Cc3nc(OCC45CCCN4CC(S(C)=O)C5)nc(N4CC5CCC(C4)N5)c3CO2)c1C(F)(F)F]

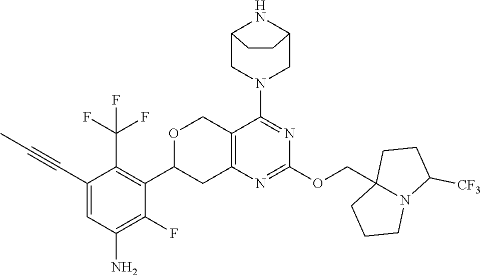 [SMILES: CC#Cc1cc(N)c(F)c(C2Cc3nc(OCC45CCCN4C(C(F)(F)F)CC5)nc(N4CC5CCC(C4)N5)c3CO2)c1C(F)(F)F]

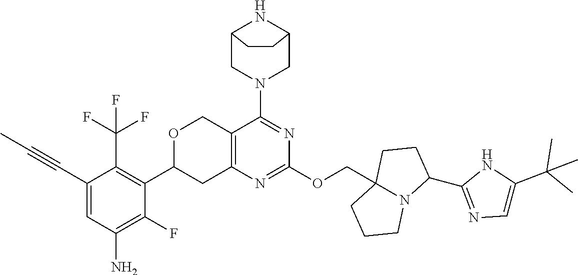 [SMILES: CC#Cc1cc(N)c(F)c(C2Cc3nc(OCC45CCCN4C(c4ncc(C(C)(C)C)[nH]4)CC5)nc(N4CC5CCC(C4)N5)c3CO2)c1C(F)(F)F]

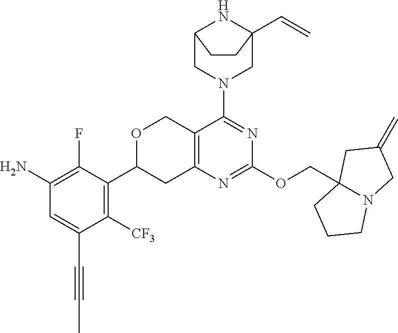 [SMILES: C=CC12CCC(CN(c3nc(OCC45CCCN4CC(=C)C5)nc4c3COC(c3c(F)c(N)cc(C#CC)c3C(F)(F)F)C4)C1)N2]

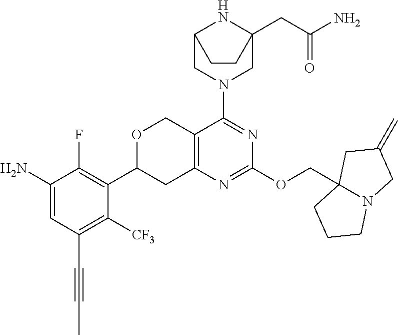 [SMILES: C=C1CN2CCCC2(COc2nc3c(c(N4CC5CCC(CC(N)=O)(C4)N5)n2)COC(c2c(F)c(N)cc(C#CC)c2C(F)(F)F)C3)C1]

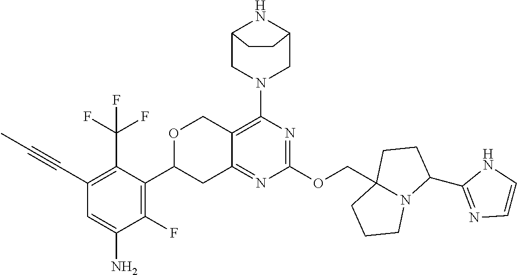 [SMILES: CC#Cc1cc(N)c(F)c(C2Cc3nc(OCC45CCCN4C(c4ncc[nH]4)CC5)nc(N4CC5CCC(C4)N5)c3CO2)c1C(F)(F)F]

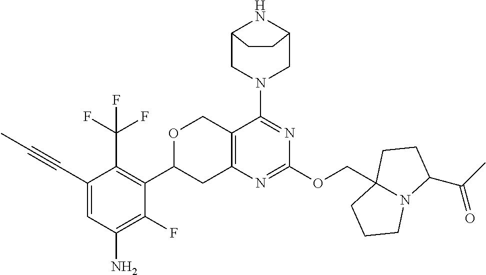 [SMILES: CC#Cc1cc(N)c(F)c(C2Cc3nc(OCC45CCCN4C(C(C)=O)CC5)nc(N4CC5CCC(C4)N5)c3CO2)c1C(F)(F)F]

In some embodiments of the present disclosure, the compound is selected from:

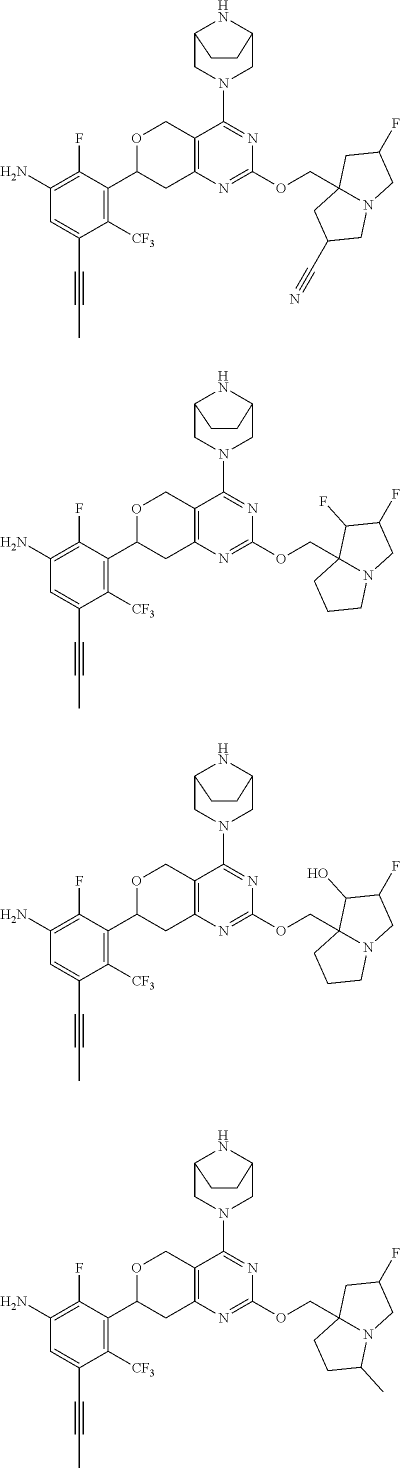 [SMILES: CC#Cc1cc(N)c(F)c(C2Cc3nc(OCC45CC(F)CN4CC(C#N)C5)nc(N4CC5CCC(C4)N5)c3CO2)c1C(F)(F)F ; CC#Cc1cc(N)c(F)c(C2Cc3nc(OCC45CCCN4CC(F)C5F)nc(N4CC5CCC(C4)N5)c3CO2)c1C(F)(F)F ; CC#Cc1cc(N)c(F)c(C2Cc3nc(OCC45CCCN4CC(F)C5O)nc(N4CC5CCC(C4)N5)c3CO2)c1C(F)(F)F ; CC#Cc1cc(N)c(F)c(C2Cc3nc(OCC45CCC(C)N4CC(F)C5)nc(N4CC5CCC(C4)N5)c3CO2)c1C(F)(F)F]

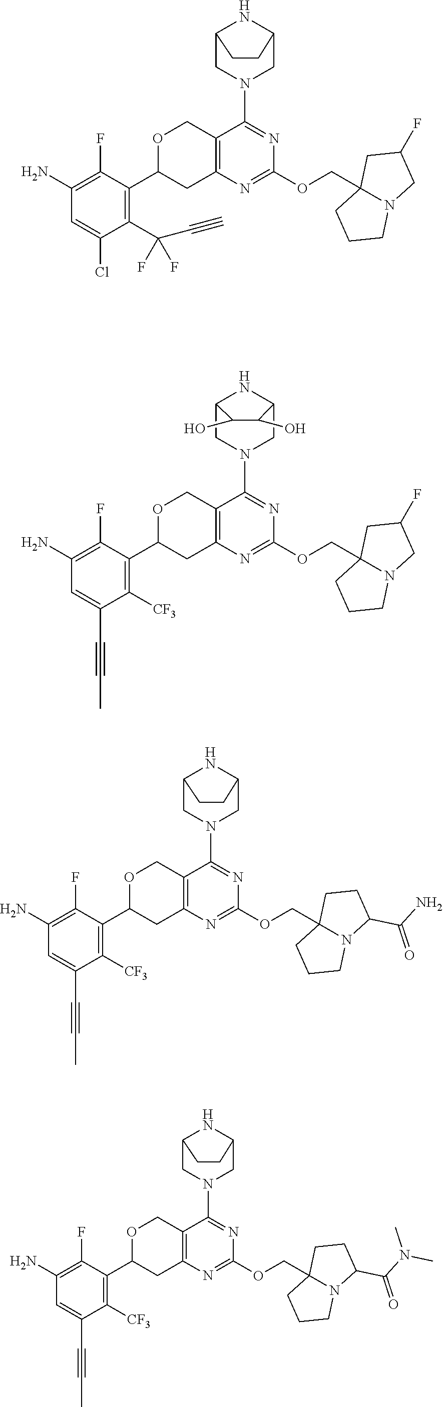 [SMILES: C#CC(F)(F)c1c(Cl)cc(N)c(F)c1C1Cc2nc(OCC34CCCN3CC(F)C4)nc(N3CC4CCC(C3)N4)c2CO1 ; CC#Cc1cc(N)c(F)c(C2Cc3nc(OCC45CCCN4CC(F)C5)nc(N4CC5NC(C4)C(O)C5O)c3CO2)c1C(F)(F)F ; CC#Cc1cc(N)c(F)c(C2Cc3nc(OCC45CCCN4C(C(N)=O)CC5)nc(N4CC5CCC(C4)N5)c3CO2)c1C(F)(F)F ; CC#Cc1cc(N)c(F)c(C2Cc3nc(OCC45CCCN4C(C(=O)N(C)C)CC5)nc(N4CC5CCC(C4)N5)c3CO2)c1C(F)(F)F]

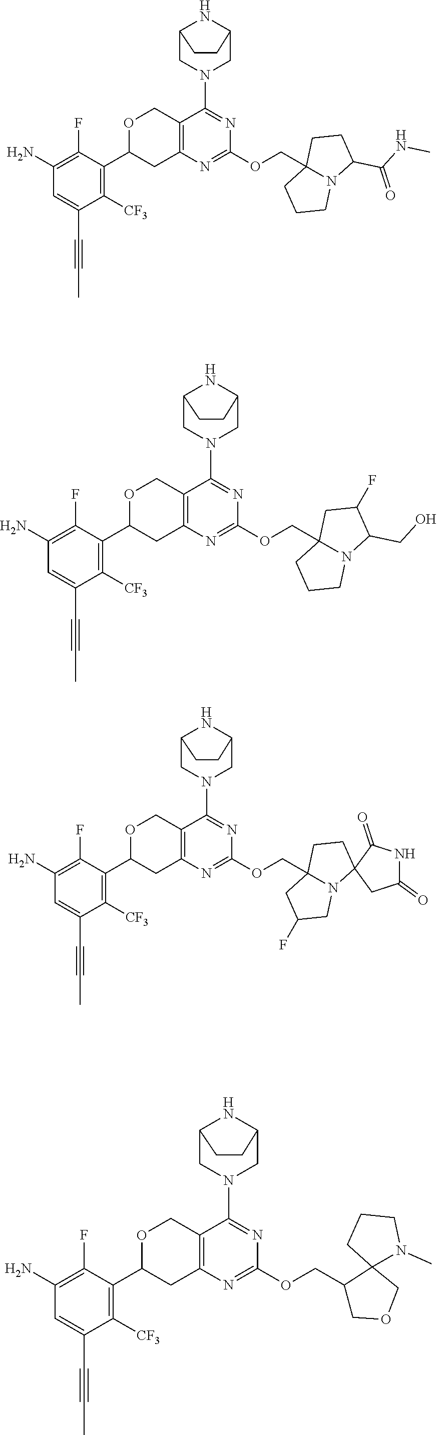 [SMILES: CC#Cc1cc(N)c(F)c(C2Cc3nc(OCC45CCCN4C(C(=O)NC)CC5)nc(N4CC5CCC(C4)N5)c3CO2)c1C(F)(F)F ; CC#Cc1cc(N)c(F)c(C2Cc3nc(OCC45CCCN4C(CO)C(F)C5)nc(N4CC5CCC(C4)N5)c3CO2)c1C(F)(F)F ; CC#Cc1cc(N)c(F)c(C2Cc3nc(OCC45CCC6(CC(=O)NC6=O)N4CC(F)C5)nc(N4CC5CCC(C4)N5)c3CO2)c1C(F)(F)F ; CC#Cc1cc(N)c(F)c(C2Cc3nc(OCC4COCC45CCCN5C)nc(N4CC5CCC(C4)N5)c3CO2)c1C(F)(F)F]

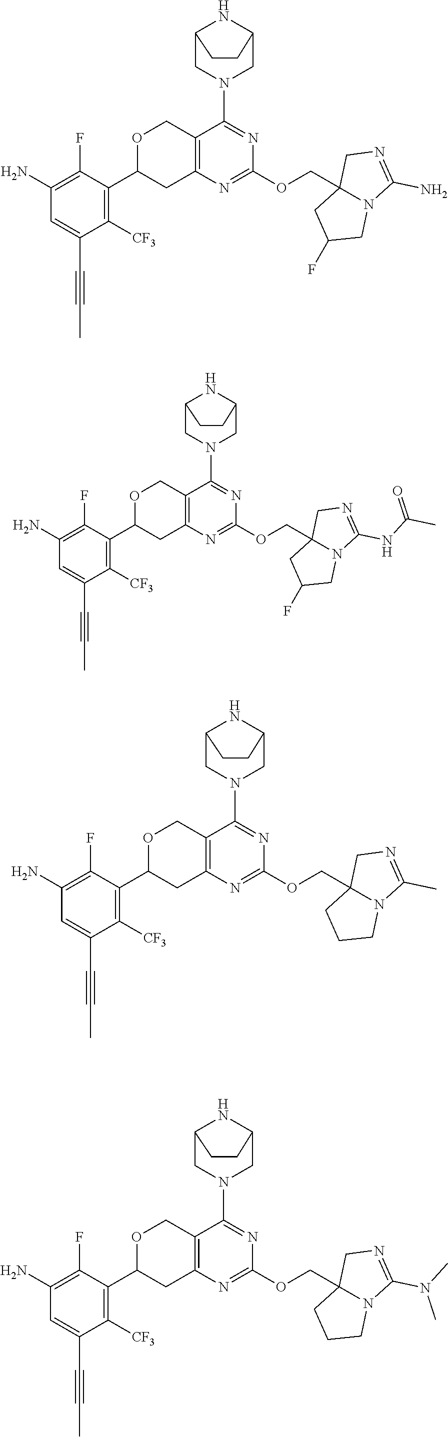 [SMILES: CC#Cc1cc(N)c(F)c(C2Cc3nc(OCC45CN=C(N)N4CC(F)C5)nc(N4CC5CCC(C4)N5)c3CO2)c1C(F)(F)F ; CC#Cc1cc(N)c(F)c(C2Cc3nc(OCC45CN=C(NC(C)=O)N4CC(F)C5)nc(N4CC5CCC(C4)N5)c3CO2)c1C(F)(F)F ; CC#Cc1cc(N)c(F)c(C2Cc3nc(OCC45CCCN4C(C)=NC5)nc(N4CC5CCC(C4)N5)c3CO2)c1C(F)(F)F ; CC#Cc1cc(N)c(F)c(C2Cc3nc(OCC45CCCN4C(N(C)C)=NC5)nc(N4CC5CCC(C4)N5)c3CO2)c1C(F)(F)F]

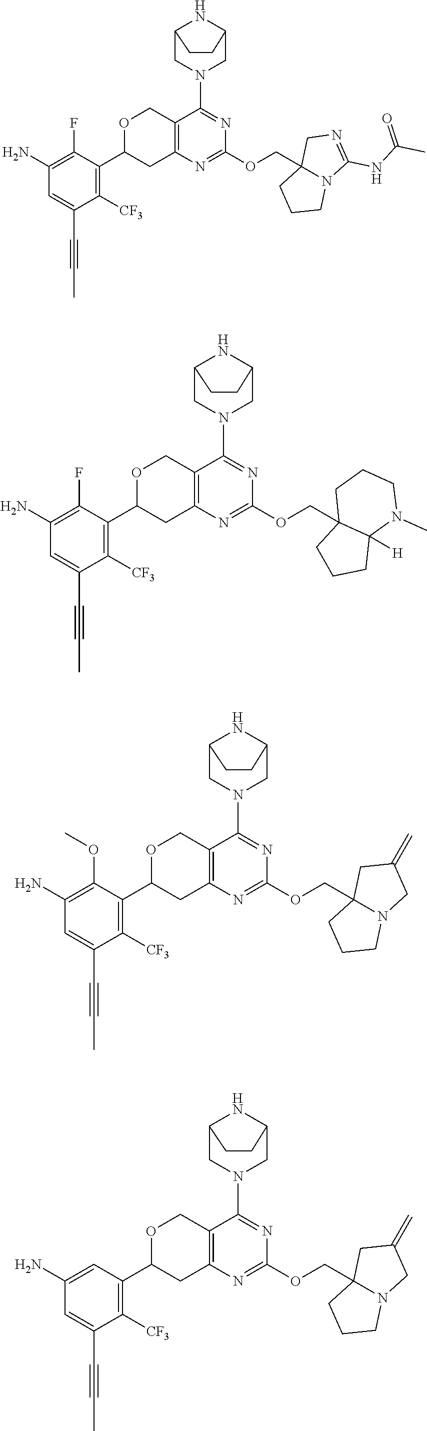 [SMILES: CC#Cc1cc(N)c(F)c(C2Cc3nc(OCC45CCCN4C(NC(C)=O)=NC5)nc(N4CC5CCC(C4)N5)c3CO2)c1C(F)(F)F ; CC#Cc1cc(N)c(F)c(C2Cc3nc(OCC45CCCC4N(C)CCC5)nc(N4CC5CCC(C4)N5)c3CO2)c1C(F)(F)F ; C=C1CN2CCCC2(COc2nc3c(c(N4CC5CCC(C4)N5)n2)COC(c2c(OC)c(N)cc(C#CC)c2C(F)(F)F)C3)C1 ; C=C1CN2CCCC2(COc2nc3c(c(N4CC5CCC(C4)N5)n2)COC(c2cc(N)cc(C#CC)c2C(F)(F)F)C3)C1]

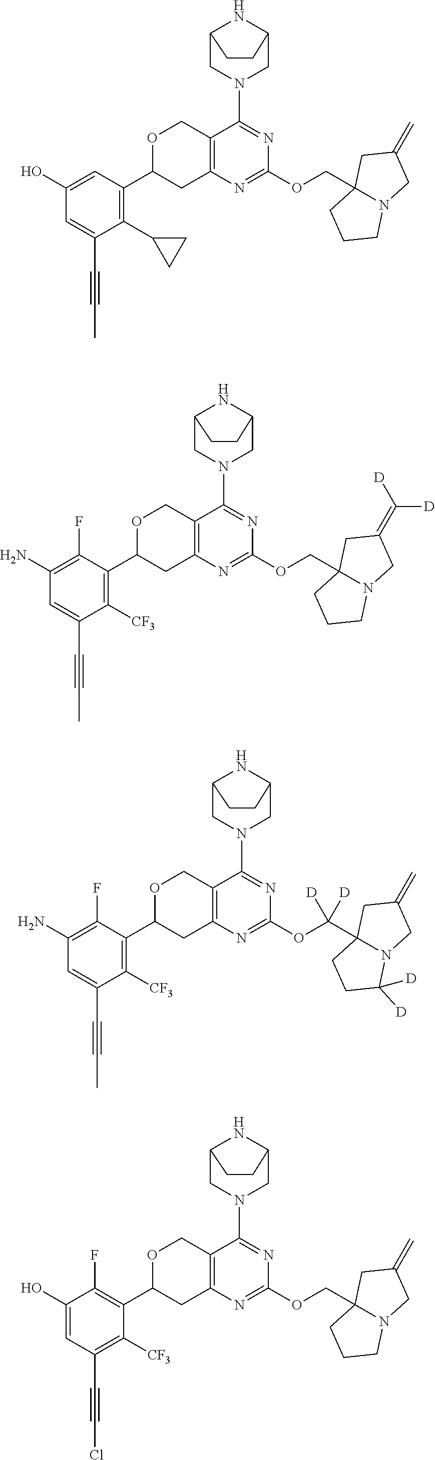 [SMILES: C=C1CN2CCCC2(COc2nc3c(c(N4CC5CCC(C4)N5)n2)COC(c2cc(O)cc(C#CC)c2C2CC2)C3)C1 ; [2H]C([2H])=C1CN2CCCC2(COc2nc3c(c(N4CC5CCC(C4)N5)n2)COC(c2c(F)c(N)cc(C#CC)c2C(F)(F)F)C3)C1 ; [2H]C1([2H])CCC2(C([2H])([2H])Oc3nc4c(c(N5CC6CCC(C5)N6)n3)COC(c3c(F)c(N)cc(C#CC)c3C(F)(F)F)C4)CC(=C)CN12 ; C=C1CN2CCCC2(COc2nc3c(c(N4CC5CCC(C4)N5)n2)COC(c2c(F)c(O)cc(C#CCl)c2C(F)(F)F)C3)C1]

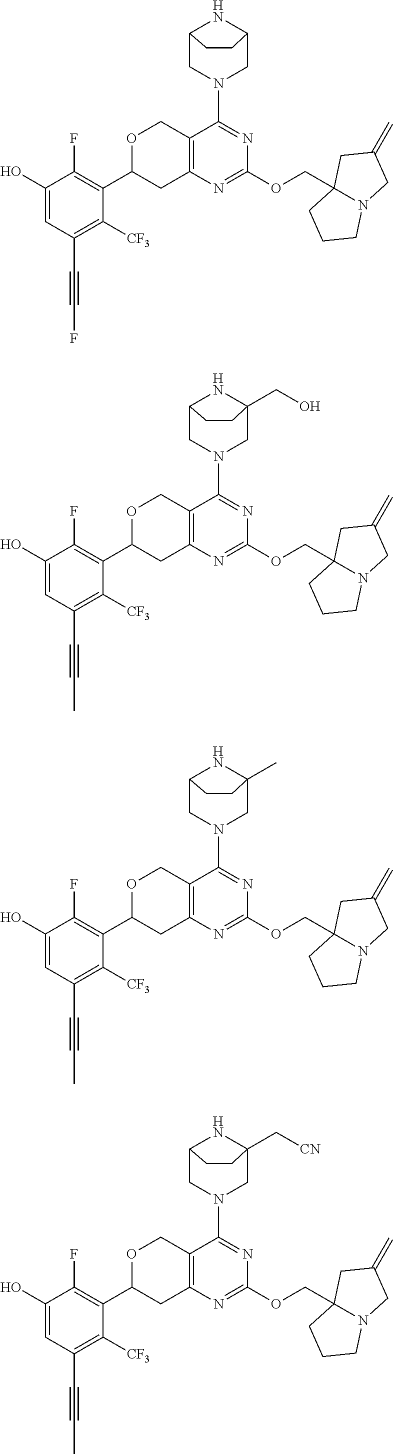 [SMILES: C=C1CN2CCCC2(COc2nc3c(c(N4CC5CCC(C4)N5)n2)COC(c2c(F)c(O)cc(C#CF)c2C(F)(F)F)C3)C1 ; C=C1CN2CCCC2(COc2nc3c(c(N4CC5CCC(CO)(C4)N5)n2)COC(c2c(F)c(O)cc(C#CC)c2C(F)(F)F)C3)C1 ; C=C1CN2CCCC2(COc2nc3c(c(N4CC5CCC(C)(C4)N5)n2)COC(c2c(F)c(O)cc(C#CC)c2C(F)(F)F)C3)C1 ; C=C1CN2CCCC2(COc2nc3c(c(N4CC5CCC(CC#N)(C4)N5)n2)COC(c2c(F)c(O)cc(C#CC)c2C(F)(F)F)C3)C1]

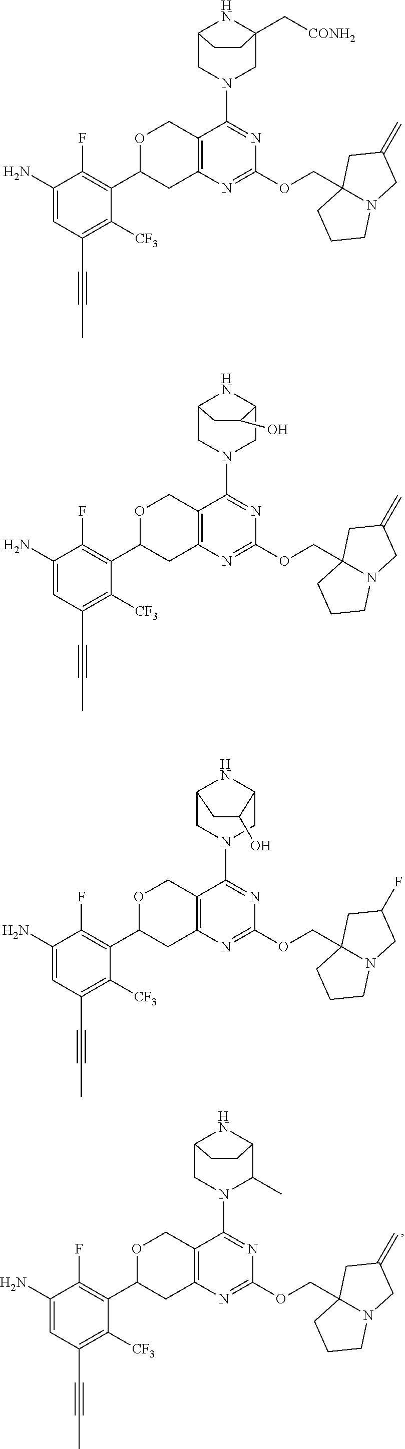 [SMILES: C=C1CN2CCCC2(COc2nc3c(c(N4CC5CCC(CC(N)=O)(C4)N5)n2)COC(c2c(F)c(N)cc(C#CC)c2C(F)(F)F)C3)C1 ; C=C1CN2CCCC2(COc2nc3c(c(N4CC5CC(O)C(C4)N5)n2)COC(c2c(F)c(N)cc(C#CC)c2C(F)(F)F)C3)C1 ; CC#Cc1cc(N)c(F)c(C2Cc3nc(OCC45CCCN4CC(F)C5)nc(N4CC5CC(O)C(C4)N5)c3CO2)c1C(F)(F)F ; C=C1CN2CCCC2(COc2nc3c(c(N4CC5CCC(N5)C4C)n2)COC(c2c(F)c(N)cc(C#CC)c2C(F)(F)F)C3)C1]

a stereoisomer thereof, or a pharmaceutically acceptable salt thereof.

In some embodiments of the present disclosure, the compound is selected from:

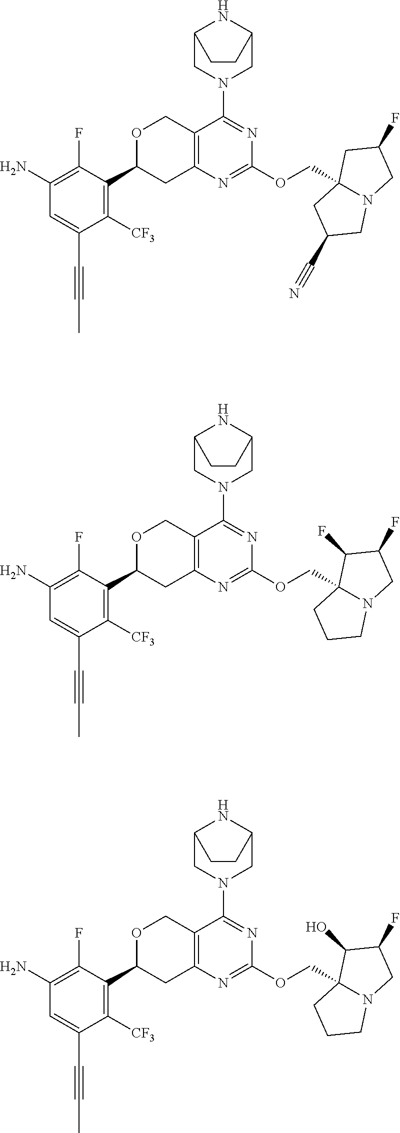 [SMILES: CC#Cc1cc(N)c(F)c([C@@H]2Cc3nc(OC[C@]45C[C@@H](F)CN4C[C@@H](C#N)C5)nc(N4CC5CCC(C4)N5)c3CO2)c1C(F)(F)F ; CC#Cc1cc(N)c(F)c([C@@H]2Cc3nc(OC[C@@]45CCCN4C[C@H](F)[C@@H]5F)nc(N4CC5CCC(C4)N5)c3CO2)c1C(F)(F)F ; CC#Cc1cc(N)c(F)c([C@@H]2Cc3nc(OC[C@@]45CCCN4C[C@H](F)[C@@H]5O)nc(N4CC5CCC(C4)N5)c3CO2)c1C(F)(F)F]

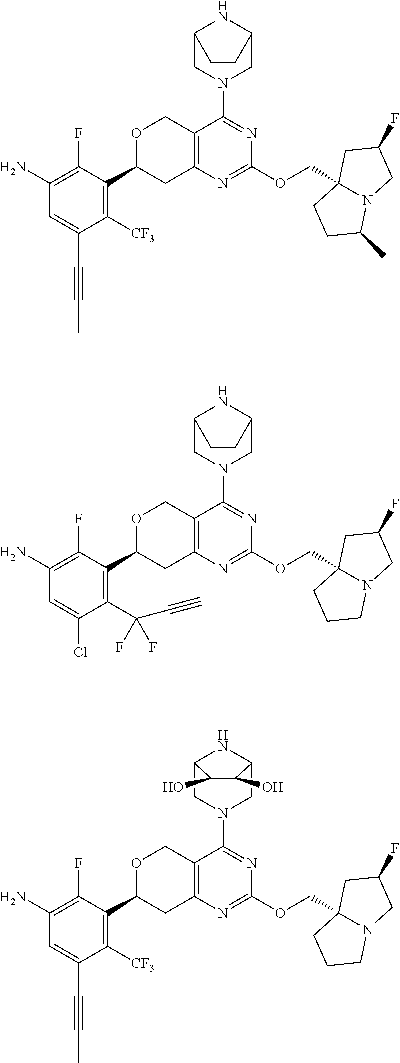 [SMILES: CC#Cc1cc(N)c(F)c([C@@H]2Cc3nc(OC[C@@]45CC[C@H](C)N4C[C@H](F)C5)nc(N4CC5CCC(C4)N5)c3CO2)c1C(F)(F)F ; C#CC(F)(F)c1c(Cl)cc(N)c(F)c1[C@@H]1Cc2nc(OC[C@@]34CCCN3C[C@H](F)C4)nc(N3CC4CCC(C3)N4)c2CO1 ; CC#Cc1cc(N)c(F)c([C@@H]2Cc3nc(OC[C@@]45CCCN4C[C@H](F)C5)nc(N4CC5NC(C4)[C@H](O)[C@@H]5O)c3CO2)c1C(F)(F)F]

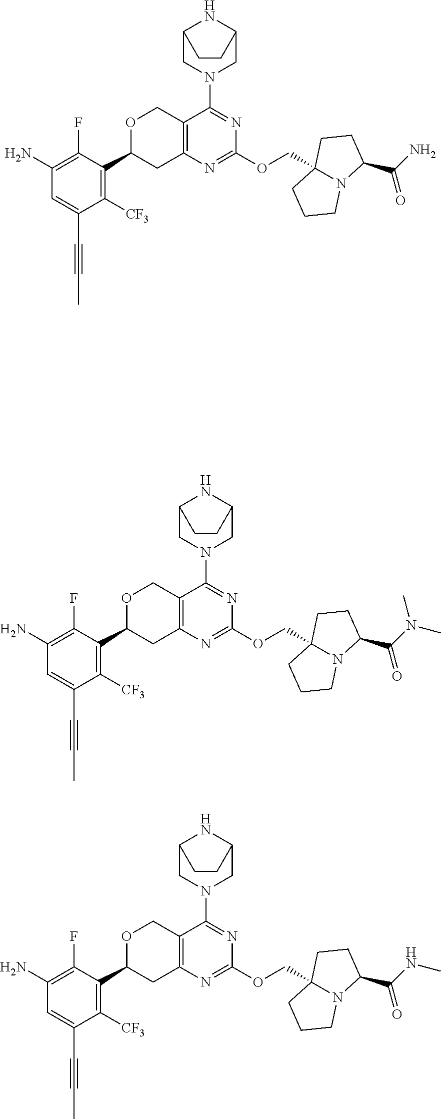 [SMILES: CC#Cc1cc(N)c(F)c([C@@H]2Cc3nc(OC[C@@]45CCCN4[C@H](C(N)=O)CC5)nc(N4CC5CCC(C4)N5)c3CO2)c1C(F)(F)F ; CC#Cc1cc(N)c(F)c([C@@H]2Cc3nc(OC[C@@]45CCCN4[C@H](C(=O)N(C)C)CC5)nc(N4CC5CCC(C4)N5)c3CO2)c1C(F)(F)F ; CC#Cc1cc(N)c(F)c([C@@H]2Cc3nc(OC[C@@]45CCCN4[C@H](C(=O)NC)CC5)nc(N4CC5CCC(C4)N5)c3CO2)c1C(F)(F)F]

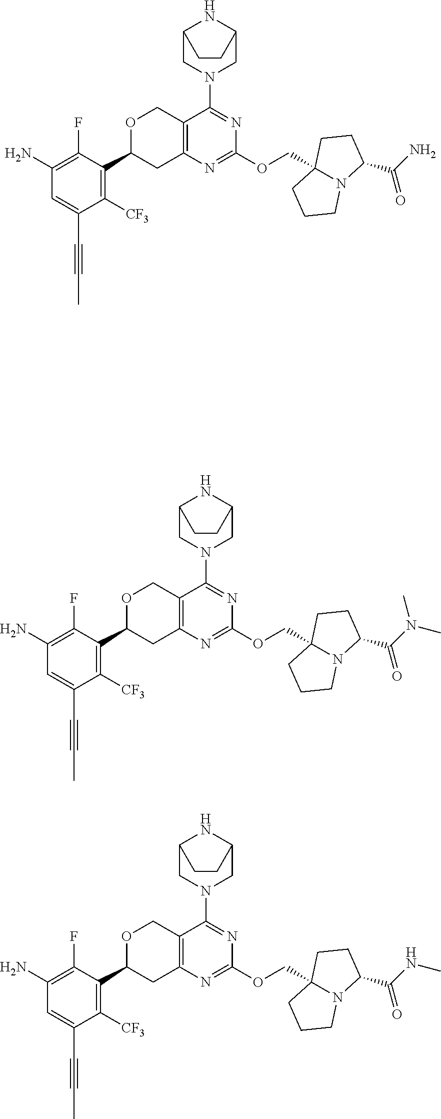 [SMILES: CC#Cc1cc(N)c(F)c([C@@H]2Cc3nc(OC[C@@]45CCCN4[C@@H](C(N)=O)CC5)nc(N4CC5CCC(C4)N5)c3CO2)c1C(F)(F)F ; CC#Cc1cc(N)c(F)c([C@@H]2Cc3nc(OC[C@@]45CCCN4[C@@H](C(=O)N(C)C)CC5)nc(N4CC5CCC(C4)N5)c3CO2)c1C(F)(F)F ; CC#Cc1cc(N)c(F)c([C@@H]2Cc3nc(OC[C@@]45CCCN4[C@@H](C(=O)NC)CC5)nc(N4CC5CCC(C4)N5)c3CO2)c1C(F)(F)F]

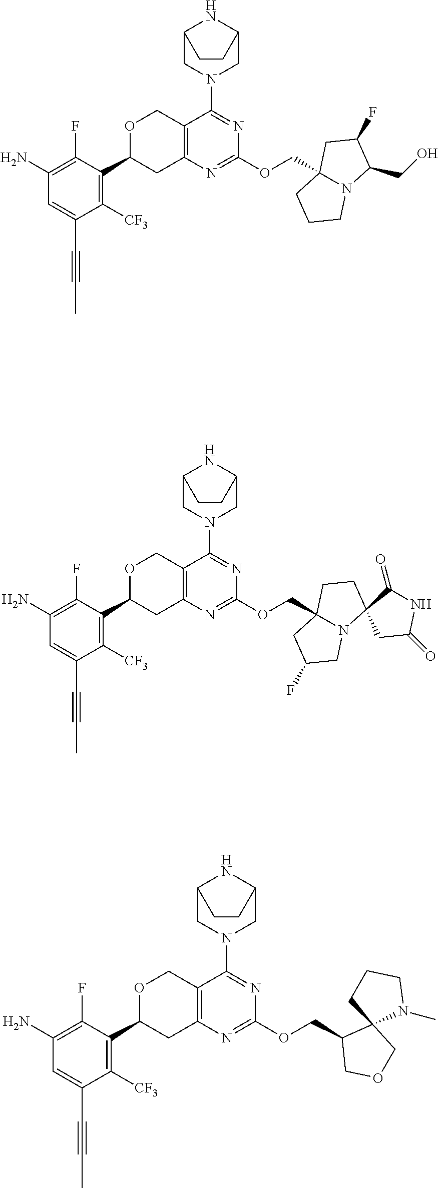 [SMILES: CC#Cc1cc(N)c(F)c([C@@H]2Cc3nc(OC[C@@]45CCCN4[C@H](CO)[C@H](F)C5)nc(N4CC5CCC(C4)N5)c3CO2)c1C(F)(F)F ; CC#Cc1cc(N)c(F)c([C@@H]2Cc3nc(OC[C@@]45CC[C@]6(CC(=O)NC6=O)N4C[C@H](F)C5)nc(N4CC5CCC(C4)N5)c3CO2)c1C(F)(F)F ; CC#Cc1cc(N)c(F)c([C@@H]2Cc3nc(OC[C@@H]4COC[C@]45CCCN5C)nc(N4CC5CCC(C4)N5)c3CO2)c1C(F)(F)F]

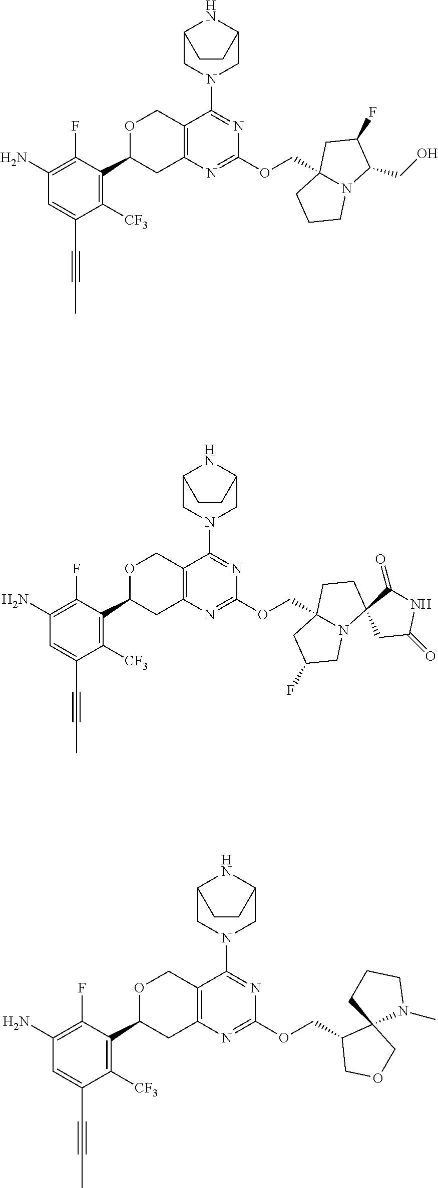 [SMILES: CC#Cc1cc(N)c(F)c([C@@H]2Cc3nc(OC[C@@]45CCCN4[C@@H](CO)[C@H](F)C5)nc(N4CC5CCC(C4)N5)c3CO2)c1C(F)(F)F ; CC#Cc1cc(N)c(F)c([C@@H]2Cc3nc(OC[C@]45CC[C@]6(CC(=O)NC6=O)N4C[C@H](F)C5)nc(N4CC5CCC(C4)N5)c3CO2)c1C(F)(F)F ; CC#Cc1cc(N)c(F)c([C@@H]2Cc3nc(OC[C@H]4COC[C@]45CCCN5C)nc(N4CC5CCC(C4)N5)c3CO2)c1C(F)(F)F]

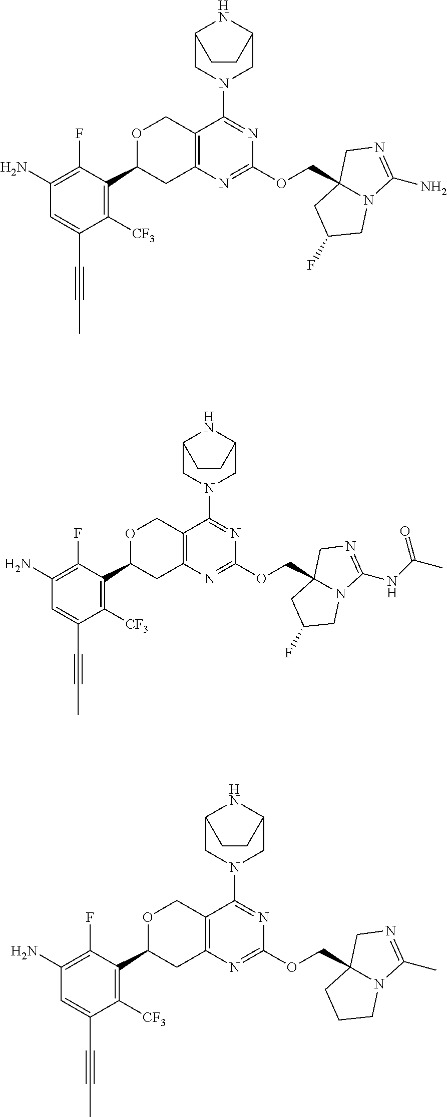 [SMILES: CC#Cc1cc(N)c(F)c([C@@H]2Cc3nc(OC[C@@]45CN=C(N)N4C[C@H](F)C5)nc(N4CC5CCC(C4)N5)c3CO2)c1C(F)(F)F ; CC#Cc1cc(N)c(F)c([C@@H]2Cc3nc(OC[C@@]45CN=C(NC(C)=O)N4C[C@H](F)C5)nc(N4CC5CCC(C4)N5)c3CO2)c1C(F)(F)F ; CC#Cc1cc(N)c(F)c([C@@H]2Cc3nc(OC[C@]45CCCN4C(C)=NC5)nc(N4CC5CCC(C4)N5)c3CO2)c1C(F)(F)F]

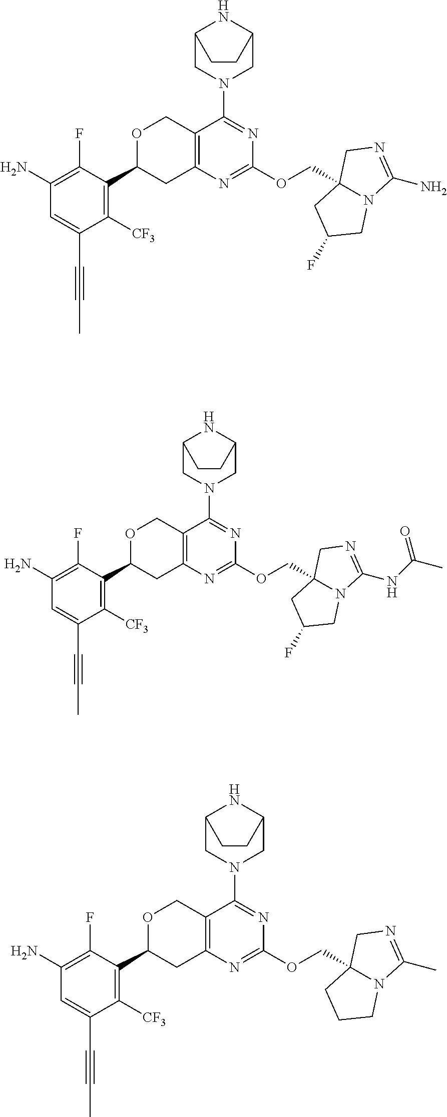 [SMILES: CC#Cc1cc(N)c(F)c([C@@H]2Cc3nc(OC[C@]45CN=C(N)N4C[C@H](F)C5)nc(N4CC5CCC(C4)N5)c3CO2)c1C(F)(F)F ; CC#Cc1cc(N)c(F)c([C@@H]2Cc3nc(OC[C@]45CN=C(NC(C)=O)N4C[C@H](F)C5)nc(N4CC5CCC(C4)N5)c3CO2)c1C(F)(F)F ; CC#Cc1cc(N)c(F)c([C@@H]2Cc3nc(OC[C@@]45CCCN4C(C)=NC5)nc(N4CC5CCC(C4)N5)c3CO2)c1C(F)(F)F]

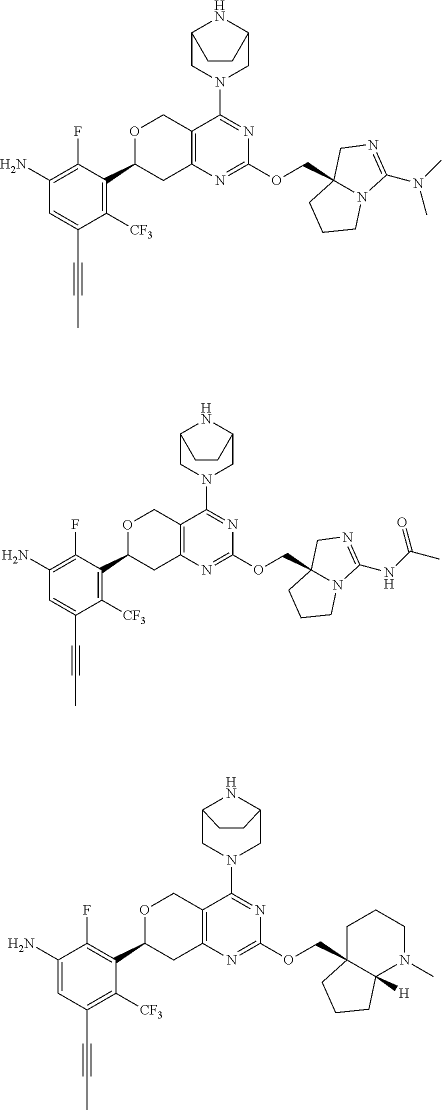 [SMILES: CC#Cc1cc(N)c(F)c([C@@H]2Cc3nc(OC[C@]45CCCN4C(N(C)C)=NC5)nc(N4CC5CCC(C4)N5)c3CO2)c1C(F)(F)F ; CC#Cc1cc(N)c(F)c([C@@H]2Cc3nc(OC[C@]45CCCN4C(NC(C)=O)=NC5)nc(N4CC5CCC(C4)N5)c3CO2)c1C(F)(F)F ; CC#Cc1cc(N)c(F)c([C@@H]2Cc3nc(OC[C@]45CCC[C@H]4N(C)CCC5)nc(N4CC5CCC(C4)N5)c3CO2)c1C(F)(F)F]

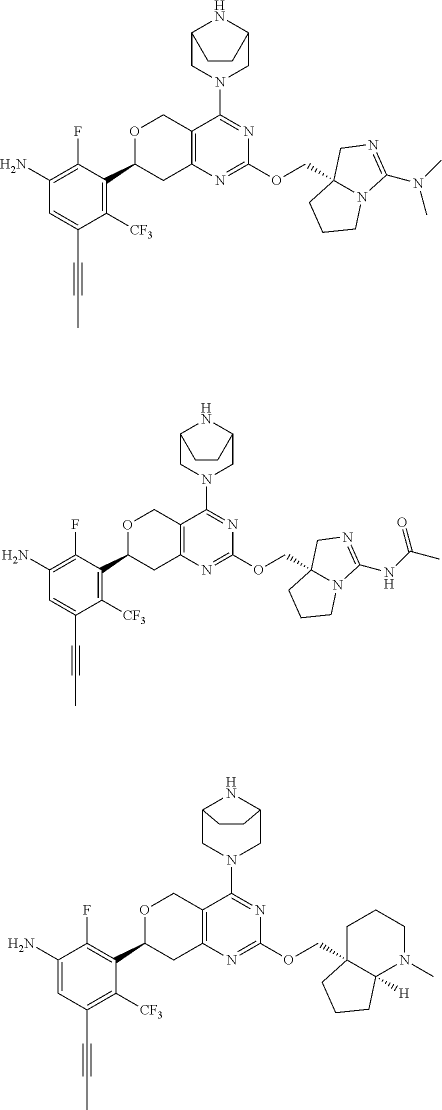 [SMILES: CC#Cc1cc(N)c(F)c([C@@H]2Cc3nc(OC[C@@]45CCCN4C(N(C)C)=NC5)nc(N4CC5CCC(C4)N5)c3CO2)c1C(F)(F)F ; CC#Cc1cc(N)c(F)c([C@@H]2Cc3nc(OC[C@@]45CCCN4C(NC(C)=O)=NC5)nc(N4CC5CCC(C4)N5)c3CO2)c1C(F)(F)F ; CC#Cc1cc(N)c(F)c([C@@H]2Cc3nc(OC[C@@]45CCC[C@@H]4N(C)CCC5)nc(N4CC5CCC(C4)N5)c3CO2)c1C(F)(F)F]

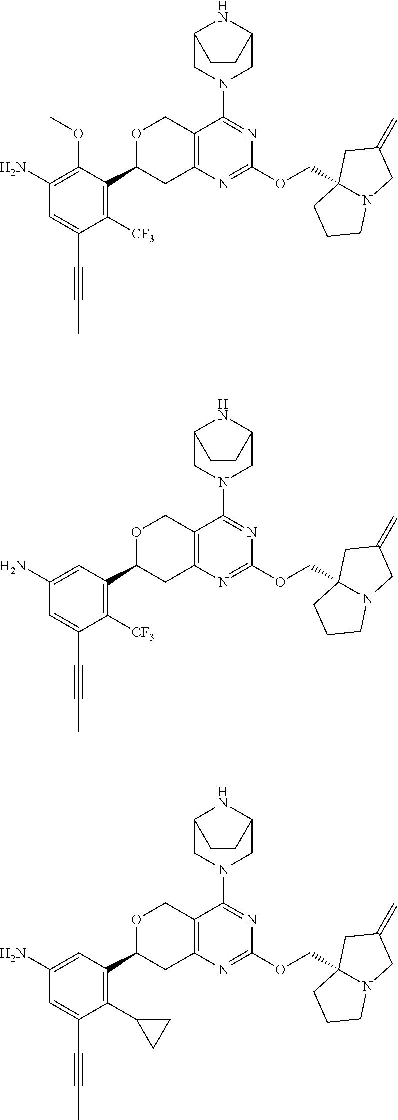 [SMILES: C=C1CN2CCC[C@@]2(COc2nc3c(c(N4CC5CCC(C4)N5)n2)CO[C@H](c2c(OC)c(N)cc(C#CC)c2C(F)(F)F)C3)C1 ; C=C1CN2CCC[C@@]2(COc2nc3c(c(N4CC5CCC(C4)N5)n2)CO[C@H](c2cc(N)cc(C#CC)c2C(F)(F)F)C3)C1 ; C=C1CN2CCC[C@@]2(COc2nc3c(c(N4CC5CCC(C4)N5)n2)CO[C@H](c2cc(N)cc(C#CC)c2C2CC2)C3)C1]

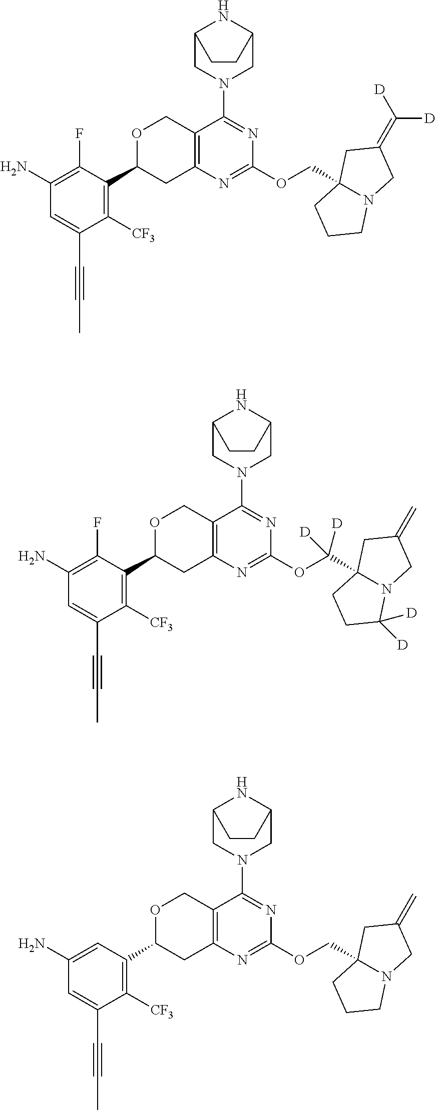 [SMILES: [2H]C([2H])=C1CN2CCC[C@@]2(COc2nc3c(c(N4CC5CCC(C4)N5)n2)CO[C@H](c2c(F)c(N)cc(C#CC)c2C(F)(F)F)C3)C1 ; [2H]C1([2H])CC[C@@]2(C([2H])([2H])Oc3nc4c(c(N5CC6CCC(C5)N6)n3)CO[C@H](c3c(F)c(N)cc(C#CC)c3C(F)(F)F)C4)CC(=C)CN12 ; C=C1CN2CCC[C@@]2(COc2nc3c(c(N4CC5CCC(C4)N5)n2)CO[C@@H](c2cc(N)cc(C#CC)c2C(F)(F)F)C3)C1]

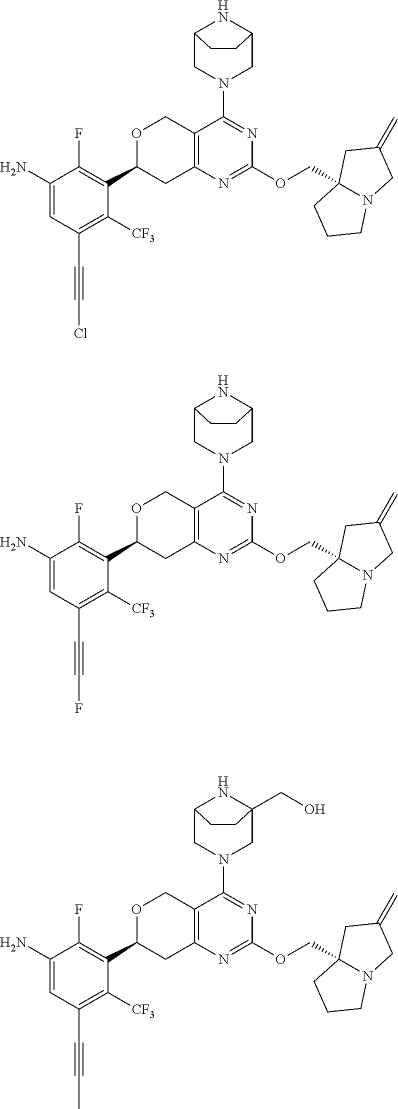 [SMILES: C=C1CN2CCC[C@@]2(COc2nc3c(c(N4CC5CCC(C4)N5)n2)CO[C@H](c2c(F)c(N)cc(C#CCl)c2C(F)(F)F)C3)C1 ; C=C1CN2CCC[C@@]2(COc2nc3c(c(N4CC5CCC(C4)N5)n2)CO[C@H](c2c(F)c(N)cc(C#CF)c2C(F)(F)F)C3)C1 ; C=C1CN2CCC[C@@]2(COc2nc3c(c(N4CC5CCC(CO)(C4)N5)n2)CO[C@H](c2c(F)c(N)cc(C#CC)c2C(F)(F)F)C3)C1]

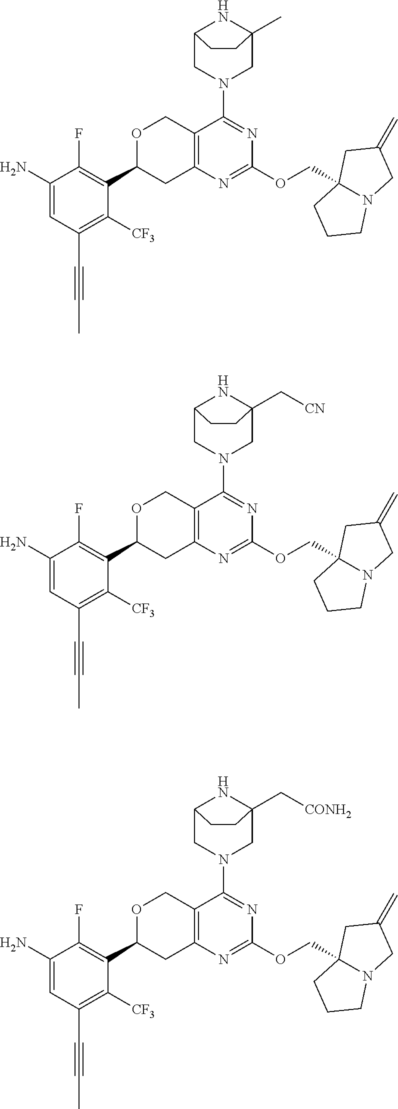 [SMILES: C=C1CN2CCC[C@@]2(COc2nc3c(c(N4CC5CCC(C)(C4)N5)n2)CO[C@H](c2c(F)c(N)cc(C#CC)c2C(F)(F)F)C3)C1 ; C=C1CN2CCC[C@@]2(COc2nc3c(c(N4CC5CCC(CC#N)(C4)N5)n2)CO[C@H](c2c(F)c(N)cc(C#CC)c2C(F)(F)F)C3)C1 ; C=C1CN2CCC[C@@]2(COc2nc3c(c(N4CC5CCC(CC(N)=O)(C4)N5)n2)CO[C@H](c2c(F)c(N)cc(C#CC)c2C(F)(F)F)C3)C1]

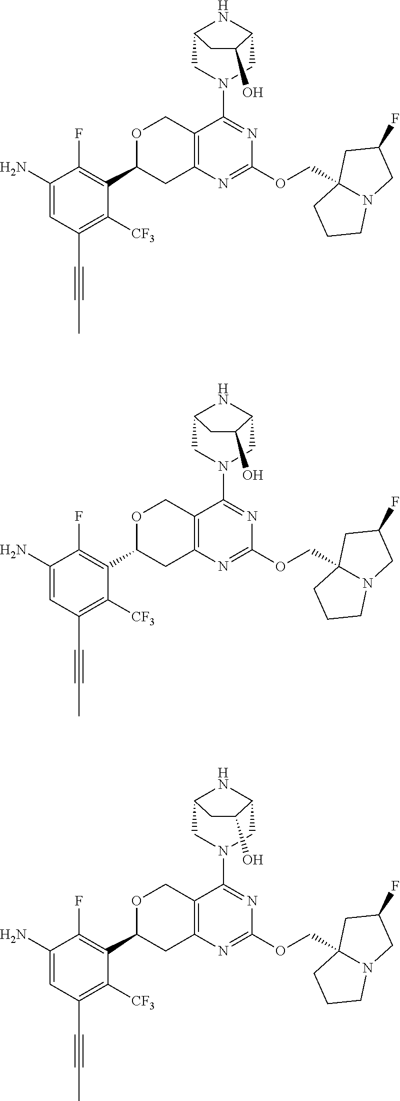 [SMILES: CC#Cc1cc(N)c(F)c([C@@H]2Cc3nc(OC[C@@]45CCCN4C[C@H](F)C5)nc(N4C[C@H]5C[C@H](O)[C@@H](C4)N5)c3CO2)c1C(F)(F)F ; CC#Cc1cc(N)c(F)c([C@H]2Cc3nc(OC[C@@]45CCCN4C[C@H](F)C5)nc(N4C[C@H]5C[C@H](O)[C@@H](C4)N5)c3CO2)c1C(F)(F)F ; CC#Cc1cc(N)c(F)c([C@@H]2Cc3nc(OC[C@@]45CCCN4C[C@H](F)C5)nc(N4C[C@H]5C[C@@H](O)[C@@H](C4)N5)c3CO2)c1C(F)(F)F]

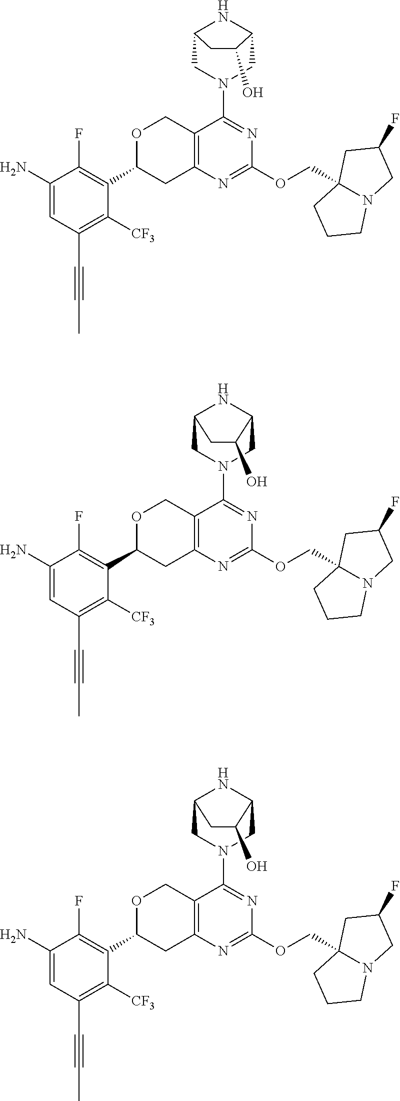 [SMILES: CC#Cc1cc(N)c(F)c([C@H]2Cc3nc(OC[C@@]45CCCN4C[C@H](F)C5)nc(N4C[C@H]5C[C@@H](O)[C@@H](C4)N5)c3CO2)c1C(F)(F)F ; CC#Cc1cc(N)c(F)c([C@@H]2Cc3nc(OC[C@@]45CCCN4C[C@H](F)C5)nc(N4C[C@@H]5C[C@H](O)[C@H](C4)N5)c3CO2)c1C(F)(F)F ; CC#Cc1cc(N)c(F)c([C@H]2Cc3nc(OC[C@@]45CCCN4C[C@H](F)C5)nc(N4C[C@@H]5C[C@H](O)[C@H](C4)N5)c3CO2)c1C(F)(F)F]

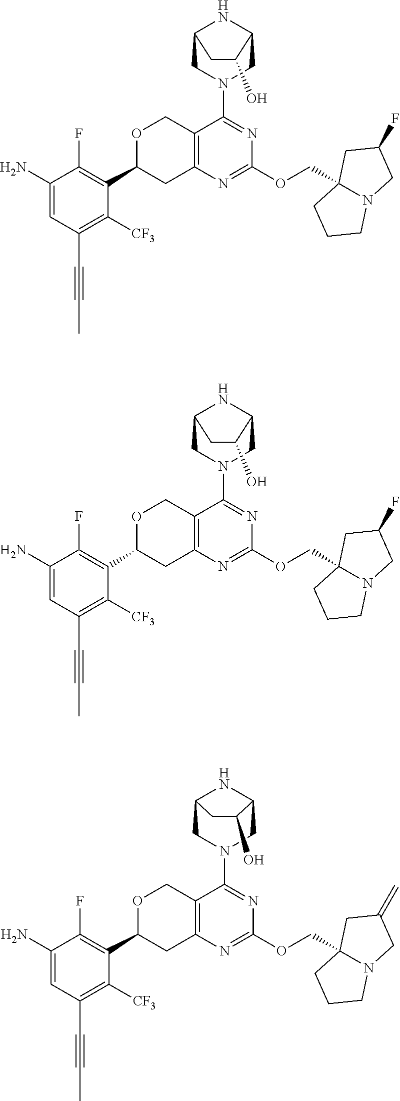 [SMILES: CC#Cc1cc(N)c(F)c([C@@H]2Cc3nc(OC[C@@]45CCCN4C[C@H](F)C5)nc(N4C[C@@H]5C[C@@H](O)[C@H](C4)N5)c3CO2)c1C(F)(F)F ; CC#Cc1cc(N)c(F)c([C@H]2Cc3nc(OC[C@@]45CCCN4C[C@H](F)C5)nc(N4C[C@@H]5C[C@@H](O)[C@H](C4)N5)c3CO2)c1C(F)(F)F ; C=C1CN2CCC[C@@]2(COc2nc3c(c(N4C[C@@H]5C[C@H](O)[C@H](C4)N5)n2)CO[C@H](c2c(F)c(N)cc(C#CC)c2C(F)(F)F)C3)C1]

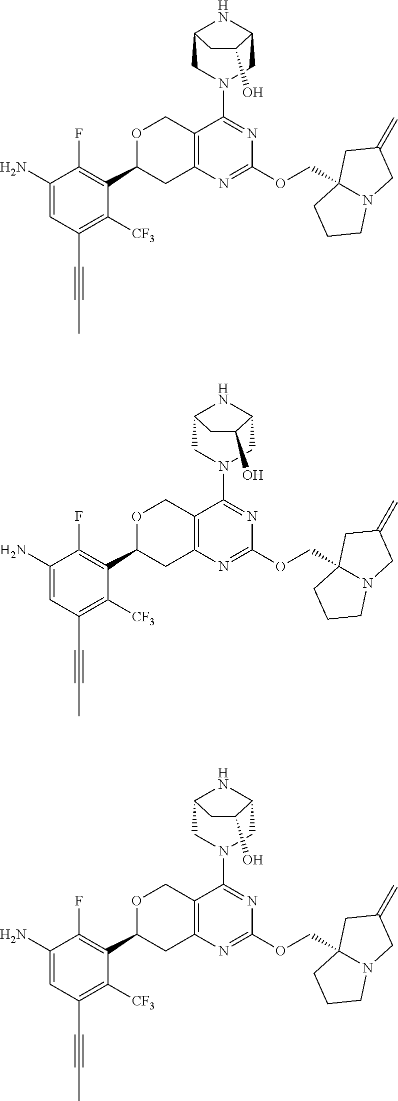 [SMILES: C=C1CN2CCC[C@@]2(COc2nc3c(c(N4C[C@@H]5C[C@@H](O)[C@H](C4)N5)n2)CO[C@H](c2c(F)c(N)cc(C#CC)c2C(F)(F)F)C3)C1 ; C=C1CN2CCC[C@@]2(COc2nc3c(c(N4C[C@H]5C[C@H](O)[C@@H](C4)N5)n2)CO[C@H](c2c(F)c(N)cc(C#CC)c2C(F)(F)F)C3)C1 ; C=C1CN2CCC[C@@]2(COc2nc3c(c(N4C[C@H]5C[C@@H](O)[C@@H](C4)N5)n2)CO[C@H](c2c(F)c(N)cc(C#CC)c2C(F)(F)F)C3)C1]

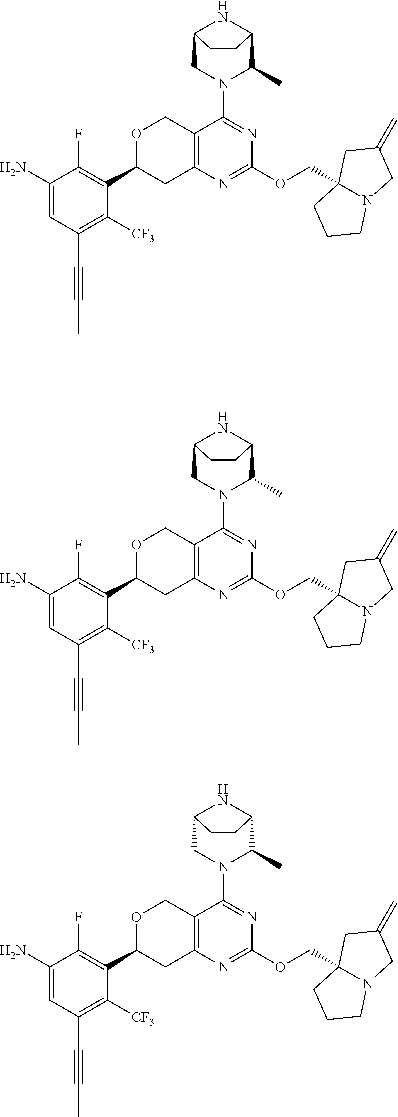 [SMILES: C=C1CN2CCC[C@@]2(COc2nc3c(c(N4C[C@@H]5CC[C@@H](N5)[C@H]4C)n2)CO[C@H](c2c(F)c(N)cc(C#CC)c2C(F)(F)F)C3)C1 ; C=C1CN2CCC[C@@]2(COc2nc3c(c(N4C[C@@H]5CC[C@@H](N5)[C@@H]4C)n2)CO[C@H](c2c(F)c(N)cc(C#CC)c2C(F)(F)F)C3)C1 ; C=C1CN2CCC[C@@]2(COc2nc3c(c(N4C[C@H]5CC[C@H](N5)[C@H]4C)n2)CO[C@H](c2c(F)c(N)cc(C#CC)c2C(F)(F)F)C3)C1]

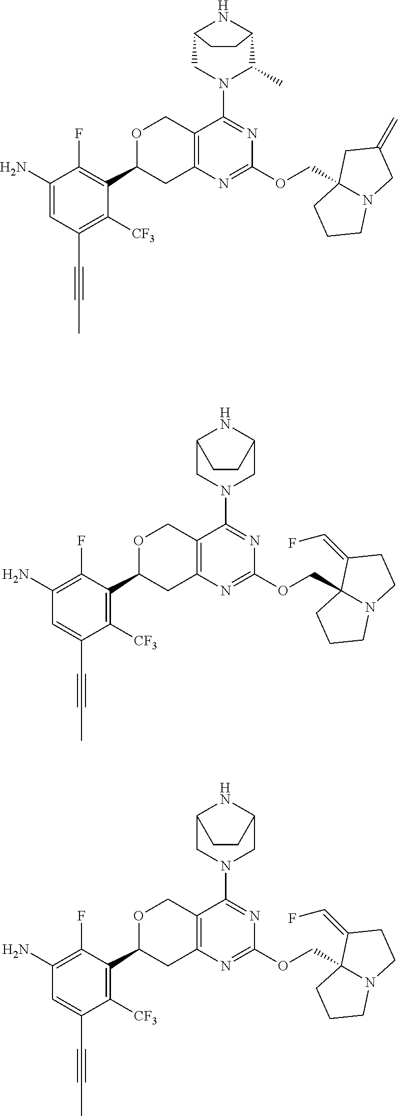 [SMILES: C=C1CN2CCC[C@@]2(COc2nc3c(c(N4C[C@H]5CC[C@H](N5)[C@@H]4C)n2)CO[C@H](c2c(F)c(N)cc(C#CC)c2C(F)(F)F)C3)C1 ; CC#Cc1cc(N)c(F)c([C@@H]2Cc3nc(OC[C@]45CCCN4CC/C5=C/F)nc(N4CC5CCC(C4)N5)c3CO2)c1C(F)(F)F ; CC#Cc1cc(N)c(F)c([C@@H]2Cc3nc(OC[C@@]45CCCN4CC/C5=C/F)nc(N4CC5CCC(C4)N5)c3CO2)c1C(F)(F)F]

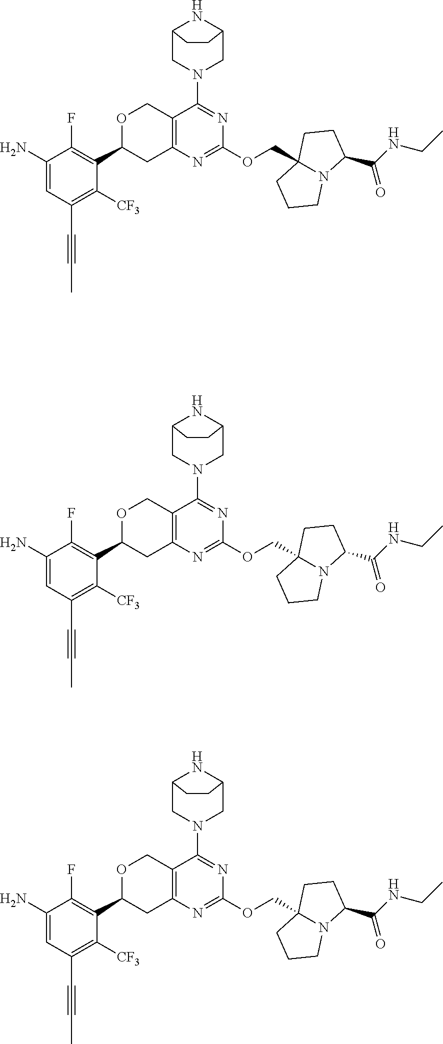 [SMILES: CC#Cc1cc(N)c(F)c([C@@H]2Cc3nc(OC[C@]45CCCN4[C@H](C(=O)NCC)CC5)nc(N4CC5CCC(C4)N5)c3CO2)c1C(F)(F)F ; CC#Cc1cc(N)c(F)c([C@@H]2Cc3nc(OC[C@@]45CCCN4[C@@H](C(=O)NCC)CC5)nc(N4CC5CCC(C4)N5)c3CO2)c1C(F)(F)F ; CC#Cc1cc(N)c(F)c([C@@H]2Cc3nc(OC[C@@]45CCCN4[C@H](C(=O)NCC)CC5)nc(N4CC5CCC(C4)N5)c3CO2)c1C(F)(F)F]

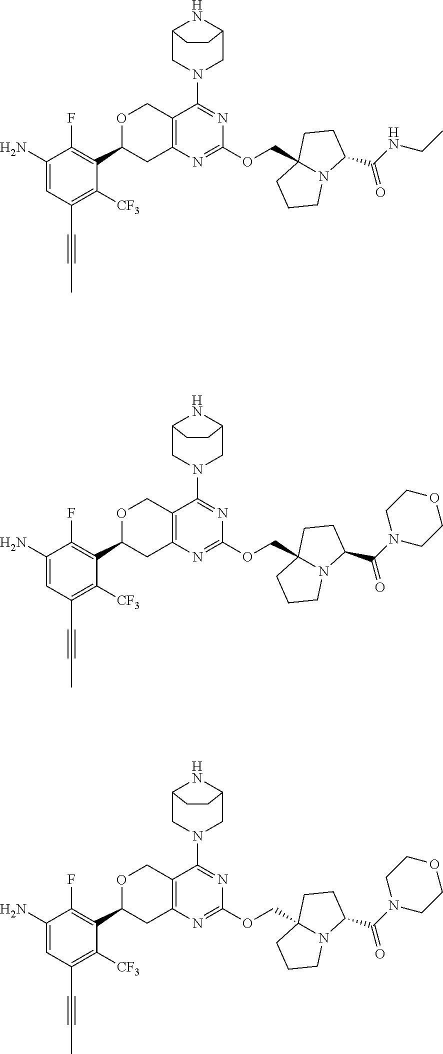 [SMILES: CC#Cc1cc(N)c(F)c([C@@H]2Cc3nc(OC[C@]45CCCN4[C@@H](C(=O)NCC)CC5)nc(N4CC5CCC(C4)N5)c3CO2)c1C(F)(F)F ; CC#Cc1cc(N)c(F)c([C@@H]2Cc3nc(OC[C@]45CCCN4[C@H](C(=O)N4CCOCC4)CC5)nc(N4CC5CCC(C4)N5)c3CO2)c1C(F)(F)F ; CC#Cc1cc(N)c(F)c([C@@H]2Cc3nc(OC[C@@]45CCCN4[C@@H](C(=O)N4CCOCC4)CC5)nc(N4CC5CCC(C4)N5)c3CO2)c1C(F)(F)F]

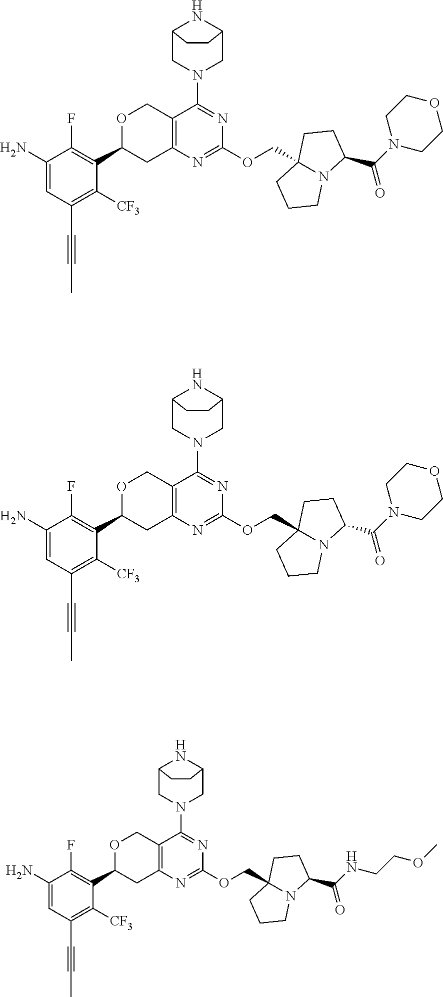 [SMILES: CC#Cc1cc(N)c(F)c([C@@H]2Cc3nc(OC[C@@]45CCCN4[C@H](C(=O)N4CCOCC4)CC5)nc(N4CC5CCC(C4)N5)c3CO2)c1C(F)(F)F ; CC#Cc1cc(N)c(F)c([C@@H]2Cc3nc(OC[C@]45CCCN4[C@@H](C(=O)N4CCOCC4)CC5)nc(N4CC5CCC(C4)N5)c3CO2)c1C(F)(F)F ; CC#Cc1cc(N)c(F)c([C@@H]2Cc3nc(OC[C@]45CCCN4[C@H](C(=O)NCCOC)CC5)nc(N4CC5CCC(C4)N5)c3CO2)c1C(F)(F)F]

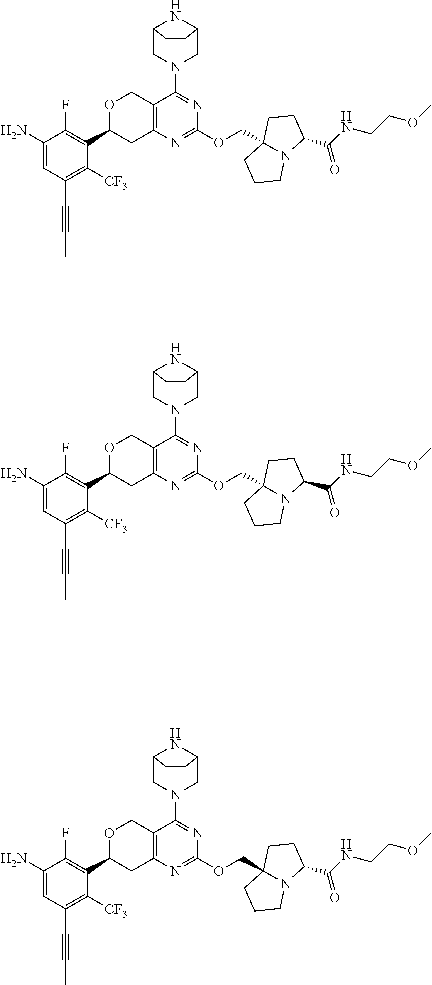 [SMILES: CC#Cc1cc(N)c(F)c([C@@H]2Cc3nc(OC[C@@]45CCCN4[C@@H](C(=O)NCCOC)CC5)nc(N4CC5CCC(C4)N5)c3CO2)c1C(F)(F)F ; CC#Cc1cc(N)c(F)c([C@@H]2Cc3nc(OC[C@@]45CCCN4[C@H](C(=O)NCCOC)CC5)nc(N4CC5CCC(C4)N5)c3CO2)c1C(F)(F)F ; CC#Cc1cc(N)c(F)c([C@@H]2Cc3nc(OC[C@]45CCCN4[C@@H](C(=O)NCCOC)CC5)nc(N4CC5CCC(C4)N5)c3CO2)c1C(F)(F)F]

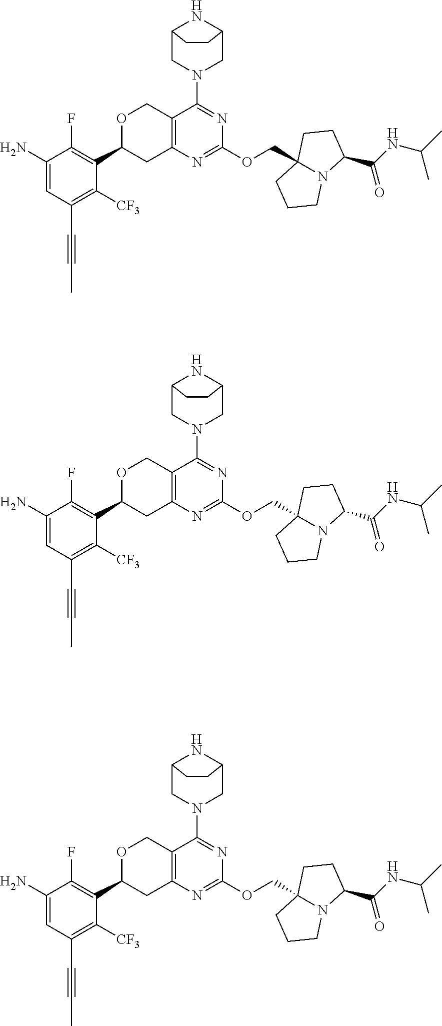 [SMILES: CC#Cc1cc(N)c(F)c([C@@H]2Cc3nc(OC[C@]45CCCN4[C@H](C(=O)NC(C)C)CC5)nc(N4CC5CCC(C4)N5)c3CO2)c1C(F)(F)F ; CC#Cc1cc(N)c(F)c([C@@H]2Cc3nc(OC[C@@]45CCCN4[C@@H](C(=O)NC(C)C)CC5)nc(N4CC5CCC(C4)N5)c3CO2)c1C(F)(F)F ; CC#Cc1cc(N)c(F)c([C@@H]2Cc3nc(OC[C@@]45CCCN4[C@H](C(=O)NC(C)C)CC5)nc(N4CC5CCC(C4)N5)c3CO2)c1C(F)(F)F]

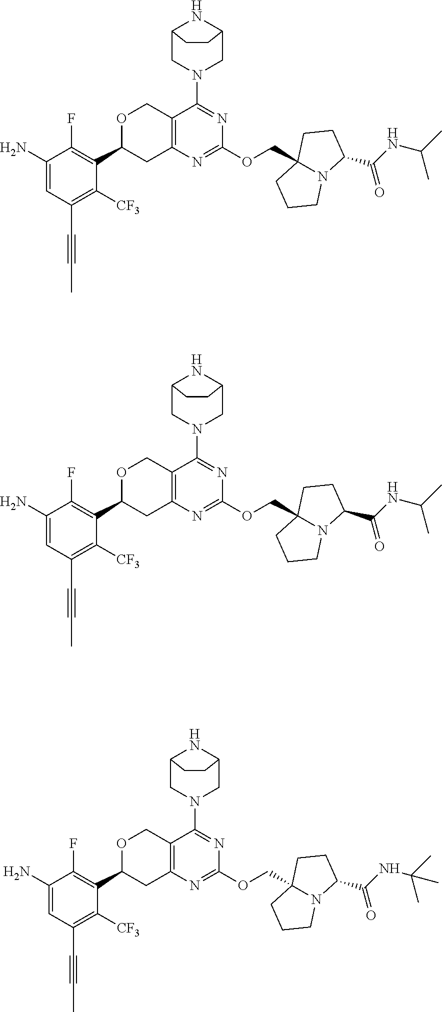 [SMILES: CC#Cc1cc(N)c(F)c([C@@H]2Cc3nc(OC[C@]45CCCN4[C@@H](C(=O)NC(C)C)CC5)nc(N4CC5CCC(C4)N5)c3CO2)c1C(F)(F)F ; CC#Cc1cc(N)c(F)c([C@@H]2Cc3nc(OC[C@]45CCCN4[C@H](C(=O)NC(C)C)CC5)nc(N4CC5CCC(C4)N5)c3CO2)c1C(F)(F)F ; CC#Cc1cc(N)c(F)c([C@@H]2Cc3nc(OC[C@@]45CCCN4[C@@H](C(=O)NC(C)(C)C)CC5)nc(N4CC5CCC(C4)N5)c3CO2)c1C(F)(F)F]

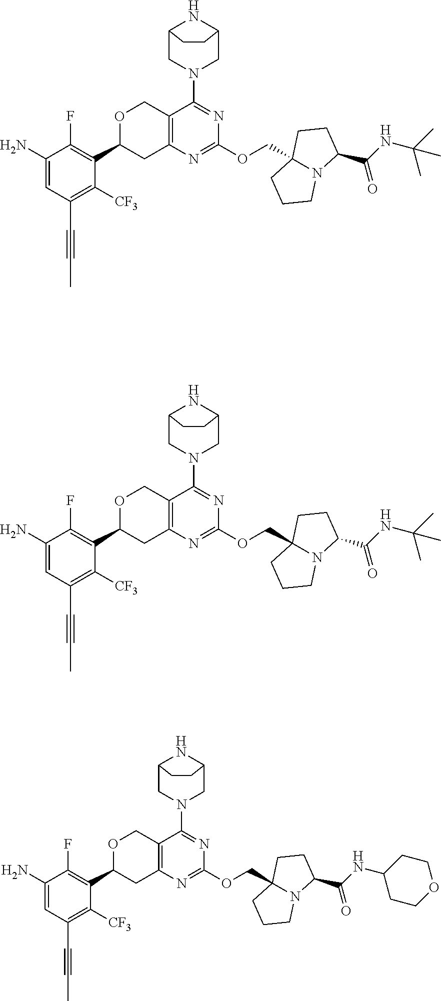 [SMILES: CC#Cc1cc(N)c(F)c([C@@H]2Cc3nc(OC[C@@]45CCCN4[C@H](C(=O)NC(C)(C)C)CC5)nc(N4CC5CCC(C4)N5)c3CO2)c1C(F)(F)F ; CC#Cc1cc(N)c(F)c([C@@H]2Cc3nc(OC[C@]45CCCN4[C@@H](C(=O)NC(C)(C)C)CC5)nc(N4CC5CCC(C4)N5)c3CO2)c1C(F)(F)F ; CC#Cc1cc(N)c(F)c([C@@H]2Cc3nc(OC[C@]45CCCN4[C@H](C(=O)NC4CCOCC4)CC5)nc(N4CC5CCC(C4)N5)c3CO2)c1C(F)(F)F]

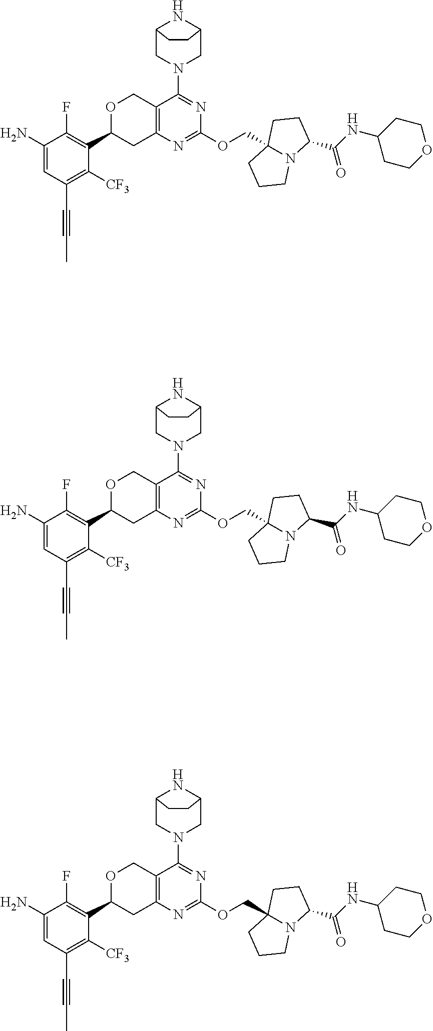 [SMILES: CC#Cc1cc(N)c(F)c([C@@H]2Cc3nc(OC[C@@]45CCCN4[C@@H](C(=O)NC4CCOCC4)CC5)nc(N4CC5CCC(C4)N5)c3CO2)c1C(F)(F)F ; CC#Cc1cc(N)c(F)c([C@@H]2Cc3nc(OC[C@@]45CCCN4[C@H](C(=O)NC4CCOCC4)CC5)nc(N4CC5CCC(C4)N5)c3CO2)c1C(F)(F)F ; CC#Cc1cc(N)c(F)c([C@@H]2Cc3nc(OC[C@]45CCCN4[C@@H](C(=O)NC4CCOCC4)CC5)nc(N4CC5CCC(C4)N5)c3CO2)c1C(F)(F)F]

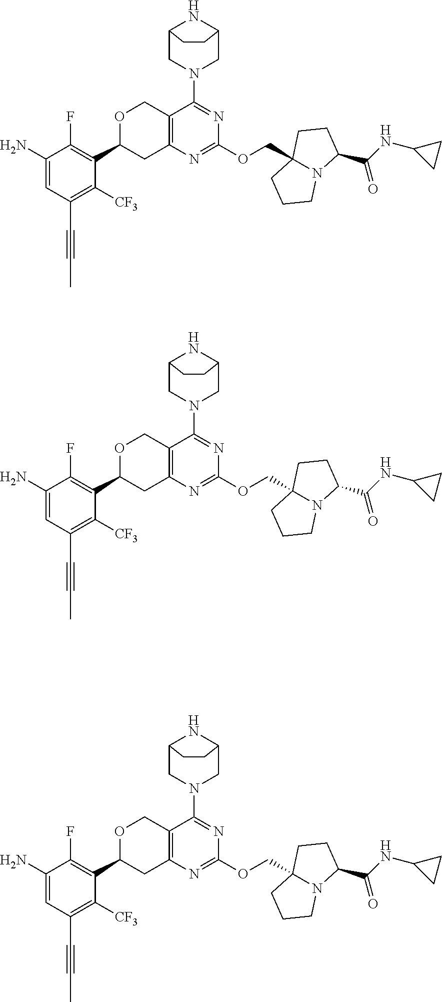 [SMILES: CC#Cc1cc(N)c(F)c([C@@H]2Cc3nc(OC[C@]45CCCN4[C@H](C(=O)NC4CC4)CC5)nc(N4CC5CCC(C4)N5)c3CO2)c1C(F)(F)F ; CC#Cc1cc(N)c(F)c([C@@H]2Cc3nc(OC[C@@]45CCCN4[C@@H](C(=O)NC4CC4)CC5)nc(N4CC5CCC(C4)N5)c3CO2)c1C(F)(F)F ; CC#Cc1cc(N)c(F)c([C@@H]2Cc3nc(OC[C@@]45CCCN4[C@H](C(=O)NC4CC4)CC5)nc(N4CC5CCC(C4)N5)c3CO2)c1C(F)(F)F]

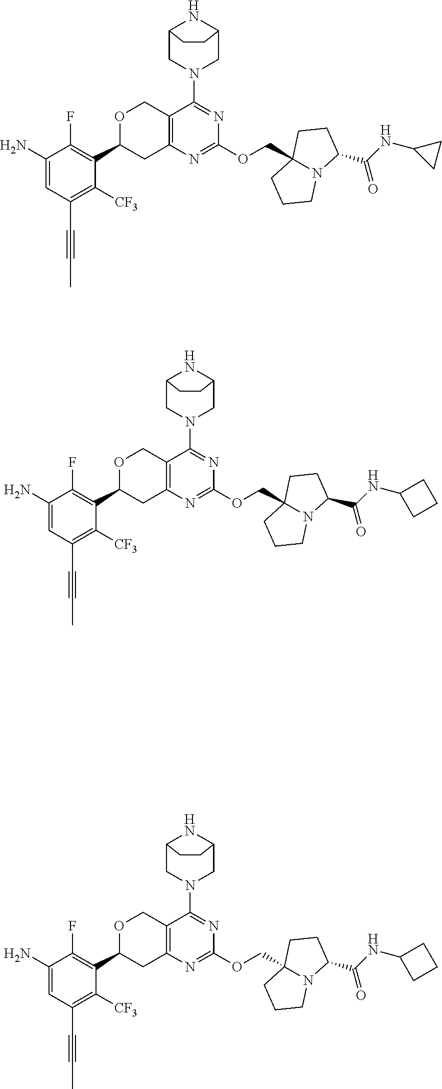 [SMILES: CC#Cc1cc(N)c(F)c([C@@H]2Cc3nc(OC[C@]45CCCN4[C@@H](C(=O)NC4CC4)CC5)nc(N4CC5CCC(C4)N5)c3CO2)c1C(F)(F)F ; CC#Cc1cc(N)c(F)c([C@@H]2Cc3nc(OC[C@]45CCCN4[C@H](C(=O)NC4CCC4)CC5)nc(N4CC5CCC(C4)N5)c3CO2)c1C(F)(F)F ; CC#Cc1cc(N)c(F)c([C@@H]2Cc3nc(OC[C@@]45CCCN4[C@@H](C(=O)NC4CCC4)CC5)nc(N4CC5CCC(C4)N5)c3CO2)c1C(F)(F)F]

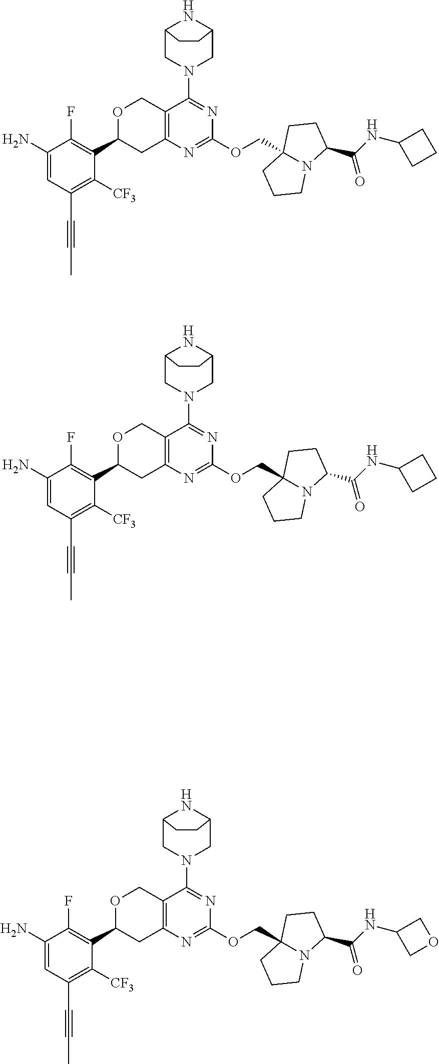 [SMILES: CC#Cc1cc(N)c(F)c([C@@H]2Cc3nc(OC[C@@]45CCCN4[C@H](C(=O)NC4CCC4)CC5)nc(N4CC5CCC(C4)N5)c3CO2)c1C(F)(F)F ; CC#Cc1cc(N)c(F)c([C@@H]2Cc3nc(OC[C@]45CCCN4[C@@H](C(=O)NC4CCC4)CC5)nc(N4CC5CCC(C4)N5)c3CO2)c1C(F)(F)F ; CC#Cc1cc(N)c(F)c([C@@H]2Cc3nc(OC[C@]45CCCN4[C@H](C(=O)NC4COC4)CC5)nc(N4CC5CCC(C4)N5)c3CO2)c1C(F)(F)F]

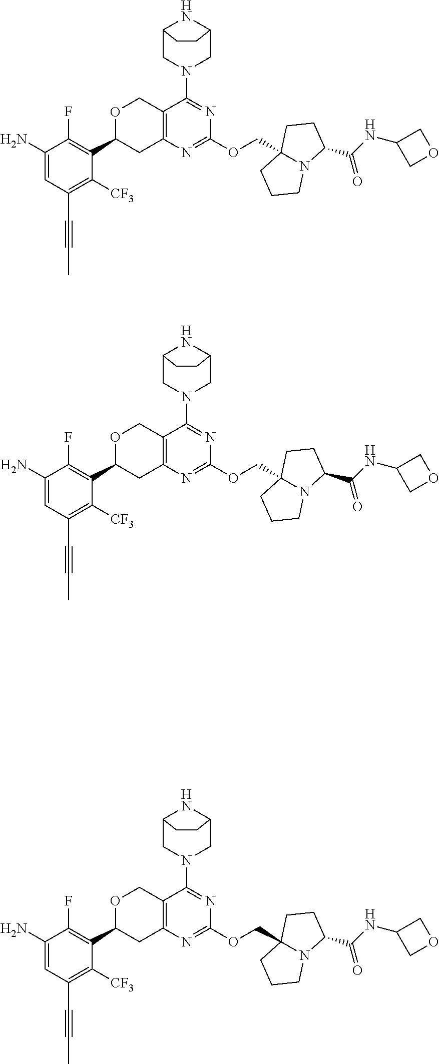 [SMILES: CC#Cc1cc(N)c(F)c([C@@H]2Cc3nc(OC[C@@]45CCCN4[C@@H](C(=O)NC4COC4)CC5)nc(N4CC5CCC(C4)N5)c3CO2)c1C(F)(F)F ; CC#Cc1cc(N)c(F)c([C@@H]2Cc3nc(OC[C@@]45CCCN4[C@H](C(=O)NC4COC4)CC5)nc(N4CC5CCC(C4)N5)c3CO2)c1C(F)(F)F ; CC#Cc1cc(N)c(F)c([C@@H]2Cc3nc(OC[C@]45CCCN4[C@@H](C(=O)NC4COC4)CC5)nc(N4CC5CCC(C4)N5)c3CO2)c1C(F)(F)F]

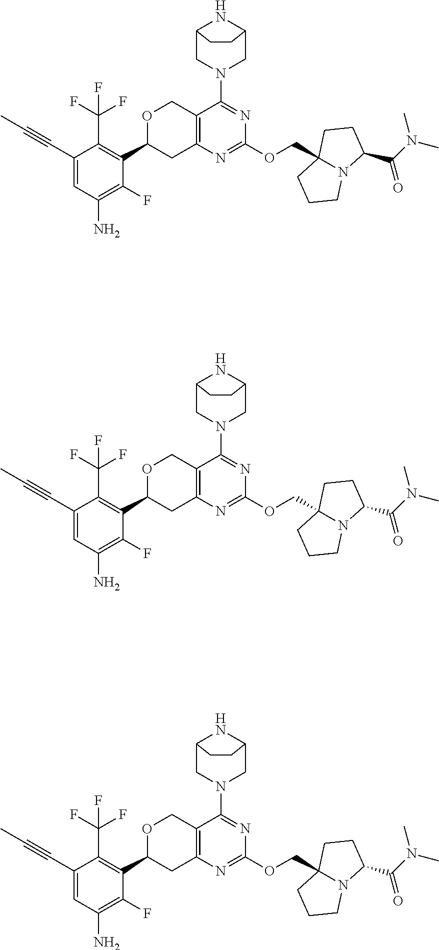

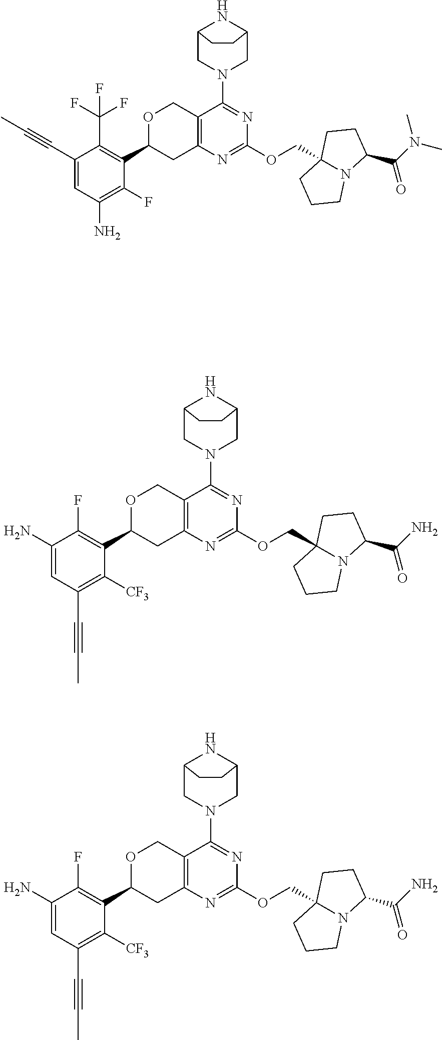 [SMILES: CC#Cc1cc(N)c(F)c([C@@H]2Cc3nc(OC[C@@]45CCCN4[C@H](C(=O)N(C)C)CC5)nc(N4CC5CCC(C4)N5)c3CO2)c1C(F)(F)F ; CC#Cc1cc(N)c(F)c([C@@H]2Cc3nc(OC[C@]45CCCN4[C@H](C(N)=O)CC5)nc(N4CC5CCC(C4)N5)c3CO2)c1C(F)(F)F ; CC#Cc1cc(N)c(F)c([C@@H]2Cc3nc(OC[C@@]45CCCN4[C@@H](C(N)=O)CC5)nc(N4CC5CCC(C4)N5)c3CO2)c1C(F)(F)F]

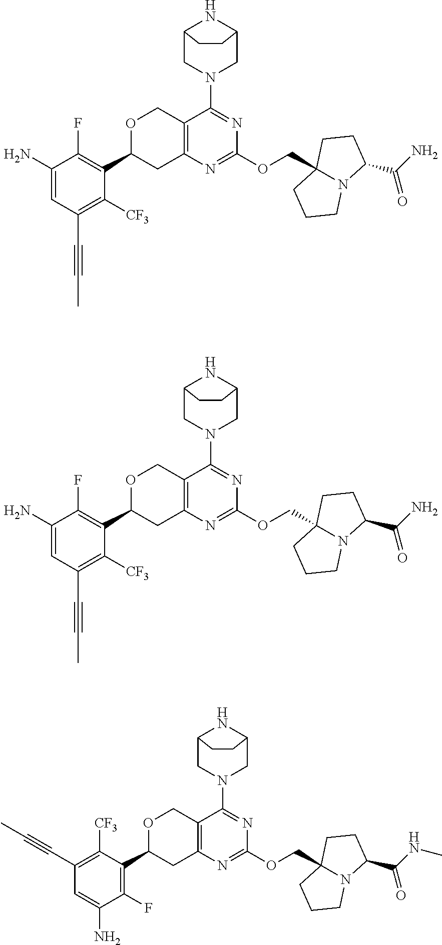 [SMILES: CC#Cc1cc(N)c(F)c([C@@H]2Cc3nc(OC[C@]45CCCN4[C@@H](C(N)=O)CC5)nc(N4CC5CCC(C4)N5)c3CO2)c1C(F)(F)F ; CC#Cc1cc(N)c(F)c([C@@H]2Cc3nc(OC[C@@]45CCCN4[C@H](C(N)=O)CC5)nc(N4CC5CCC(C4)N5)c3CO2)c1C(F)(F)F ; CC#Cc1cc(N)c(F)c([C@@H]2Cc3nc(OC[C@]45CCCN4[C@H](C(=O)NC)CC5)nc(N4CC5CCC(C4)N5)c3CO2)c1C(F)(F)F]

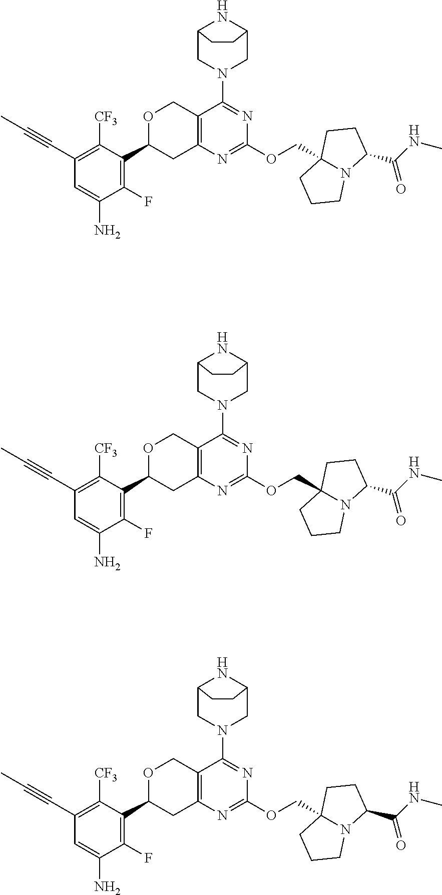

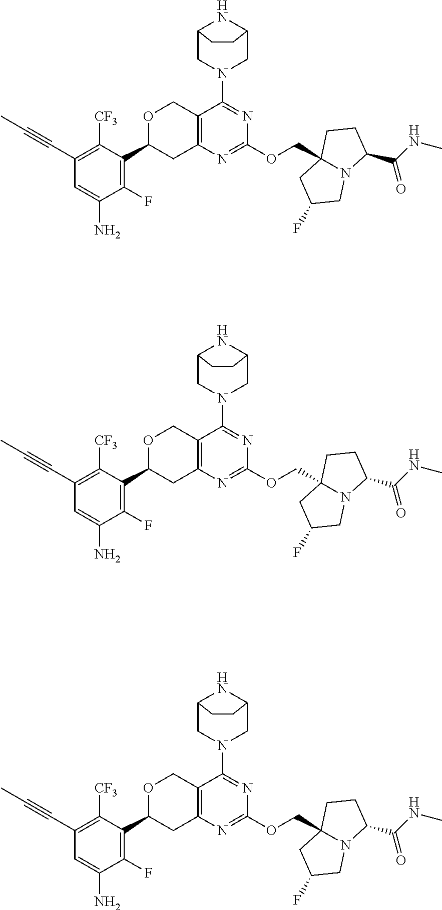

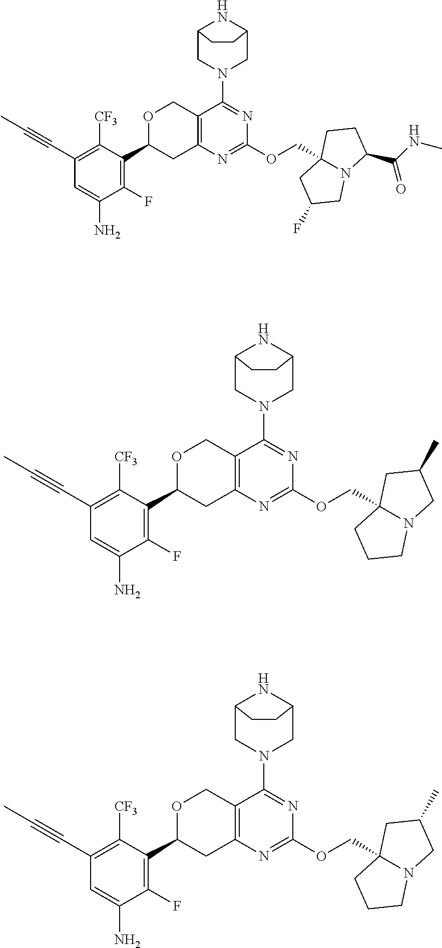

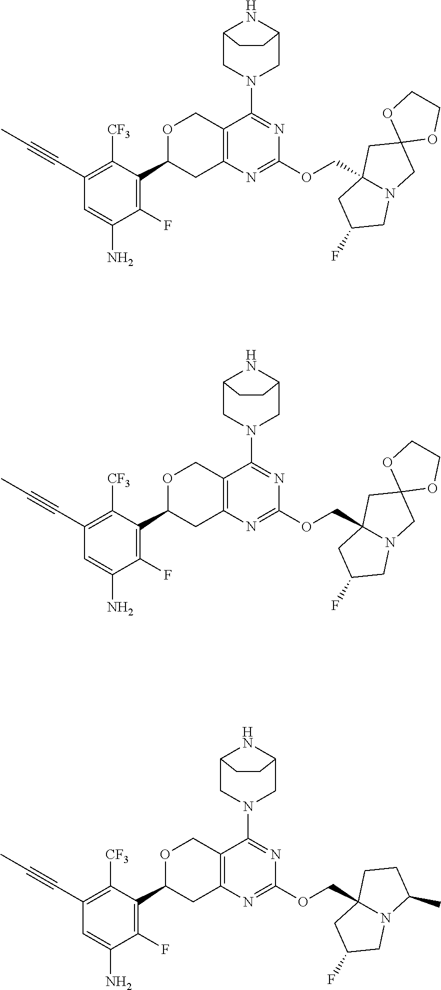 [SMILES: CC#Cc1cc(N)c(F)c([C@@H]2Cc3nc(OC[C@@]45C[C@@H](F)CN4CC4(C5)OCCO4)nc(N4CC5CCC(C4)N5)c3CO2)c1C(F)(F)F ; CC#Cc1cc(N)c(F)c([C@@H]2Cc3nc(OC[C@]45C[C@@H](F)CN4CC4(C5)OCCO4)nc(N4CC5CCC(C4)N5)c3CO2)c1C(F)(F)F ; CC#Cc1cc(N)c(F)c([C@@H]2Cc3nc(OC[C@@]45CC[C@@H](C)N4C[C@H](F)C5)nc(N4CC5CCC(C4)N5)c3CO2)c1C(F)(F)F]

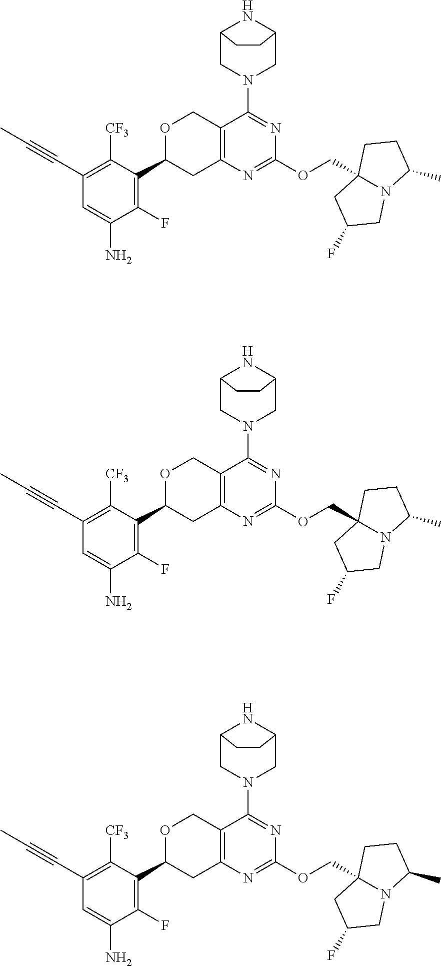

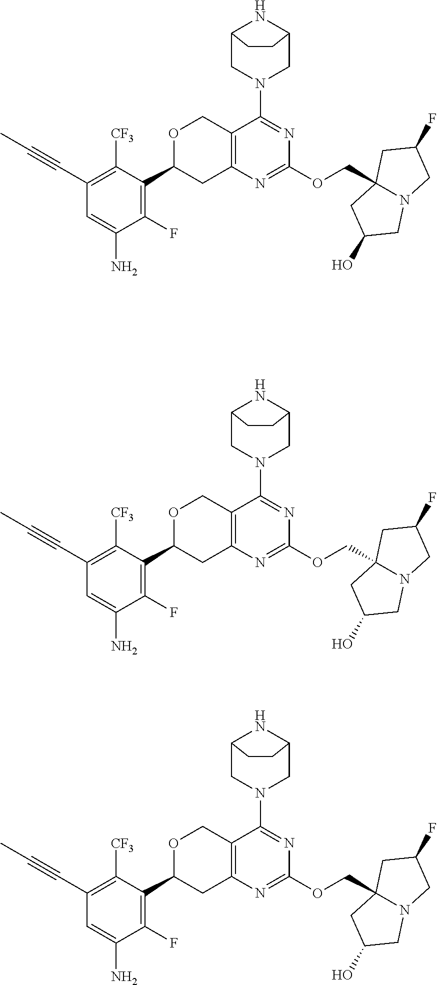 [SMILES: CC#Cc1cc(N)c(F)c([C@@H]2Cc3nc(OC[C@]45C[C@H](O)CN4C[C@H](F)C5)nc(N4CC5CCC(C4)N5)c3CO2)c1C(F)(F)F ; CC#Cc1cc(N)c(F)c([C@@H]2Cc3nc(OC[C@@]45C[C@@H](O)CN4C[C@H](F)C5)nc(N4CC5CCC(C4)N5)c3CO2)c1C(F)(F)F ; CC#Cc1cc(N)c(F)c([C@@H]2Cc3nc(OC[C@]45C[C@@H](O)CN4C[C@H](F)C5)nc(N4CC5CCC(C4)N5)c3CO2)c1C(F)(F)F]

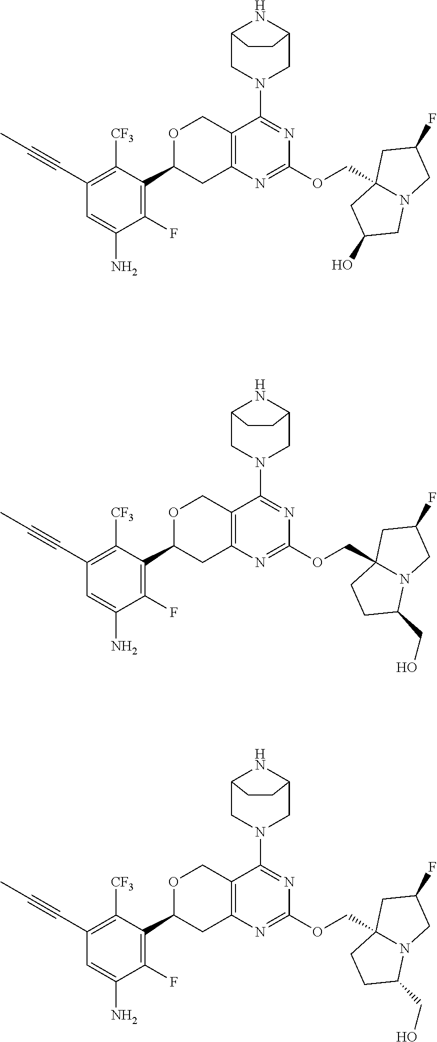 [SMILES: CC#Cc1cc(N)c(F)c([C@@H]2Cc3nc(OC[C@@]45C[C@H](O)CN4C[C@H](F)C5)nc(N4CC5CCC(C4)N5)c3CO2)c1C(F)(F)F ; CC#Cc1cc(N)c(F)c([C@@H]2Cc3nc(OC[C@]45CC[C@H](CO)N4C[C@H](F)C5)nc(N4CC5CCC(C4)N5)c3CO2)c1C(F)(F)F ; CC#Cc1cc(N)c(F)c([C@@H]2Cc3nc(OC[C@@]45CC[C@@H](CO)N4C[C@H](F)C5)nc(N4CC5CCC(C4)N5)c3CO2)c1C(F)(F)F]

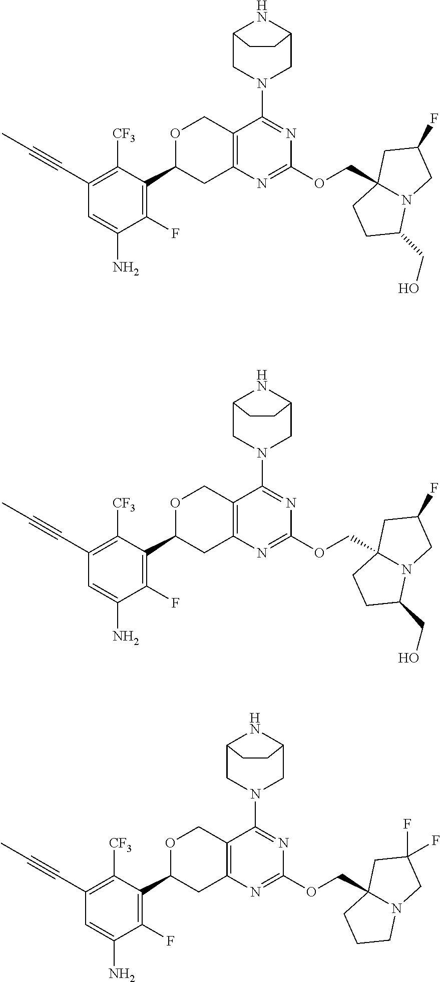 [SMILES: CC#Cc1cc(N)c(F)c([C@@H]2Cc3nc(OC[C@]45CC[C@@H](CO)N4C[C@H](F)C5)nc(N4CC5CCC(C4)N5)c3CO2)c1C(F)(F)F ; CC#Cc1cc(N)c(F)c([C@@H]2Cc3nc(OC[C@@]45CC[C@H](CO)N4C[C@H](F)C5)nc(N4CC5CCC(C4)N5)c3CO2)c1C(F)(F)F ; CC#Cc1cc(N)c(F)c([C@@H]2Cc3nc(OC[C@]45CCCN4CC(F)(F)C5)nc(N4CC5CCC(C4)N5)c3CO2)c1C(F)(F)F]

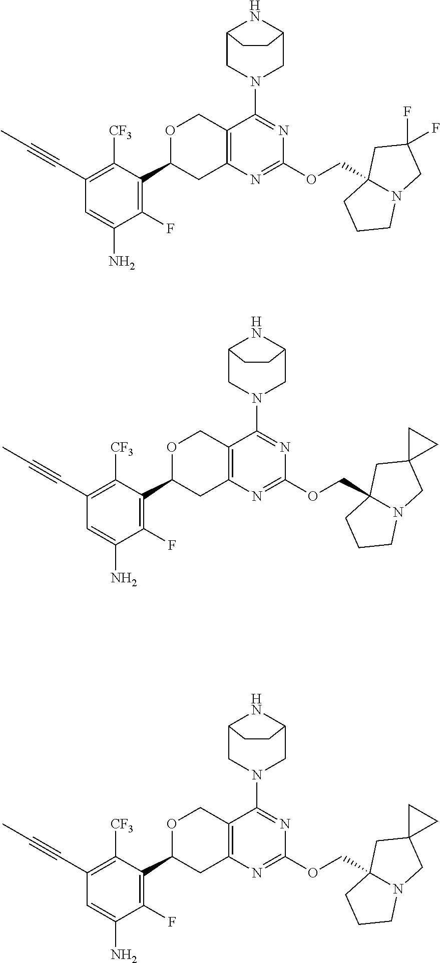 [SMILES: CC#Cc1cc(N)c(F)c([C@@H]2Cc3nc(OC[C@@]45CCCN4CC(F)(F)C5)nc(N4CC5CCC(C4)N5)c3CO2)c1C(F)(F)F ; CC#Cc1cc(N)c(F)c([C@@H]2Cc3nc(OC[C@]45CCCN4CC4(CC4)C5)nc(N4CC5CCC(C4)N5)c3CO2)c1C(F)(F)F ; CC#Cc1cc(N)c(F)c([C@@H]2Cc3nc(OC[C@@]45CCCN4CC4(CC4)C5)nc(N4CC5CCC(C4)N5)c3CO2)c1C(F)(F)F]

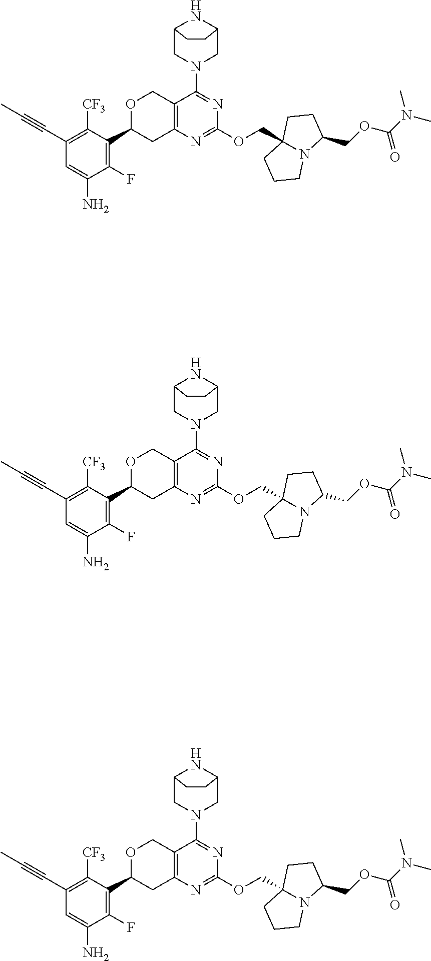

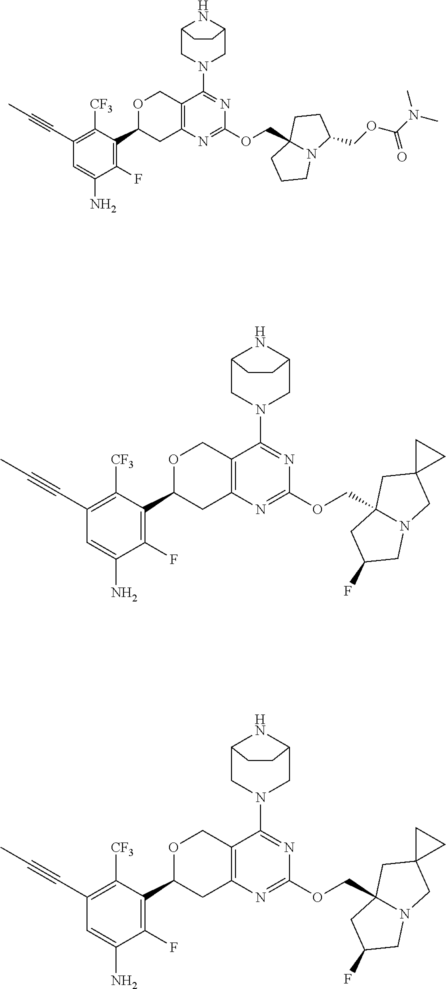

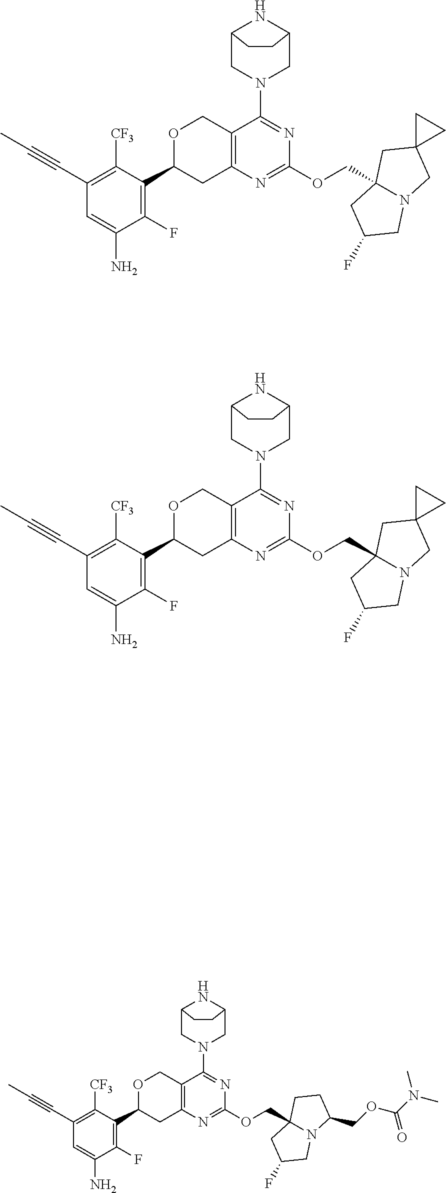 [SMILES: CC#Cc1cc(N)c(F)c([C@@H]2Cc3nc(OC[C@@]45C[C@@H](F)CN4CC4(CC4)C5)nc(N4CC5CCC(C4)N5)c3CO2)c1C(F)(F)F ; CC#Cc1cc(N)c(F)c([C@@H]2Cc3nc(OC[C@]45C[C@@H](F)CN4CC4(CC4)C5)nc(N4CC5CCC(C4)N5)c3CO2)c1C(F)(F)F ; CC#Cc1cc(N)c(F)c([C@@H]2Cc3nc(OC[C@@]45CC[C@@H](COC(=O)N(C)C)N4C[C@H](F)C5)nc(N4CC5CCC(C4)N5)c3CO2)c1C(F)(F)F]

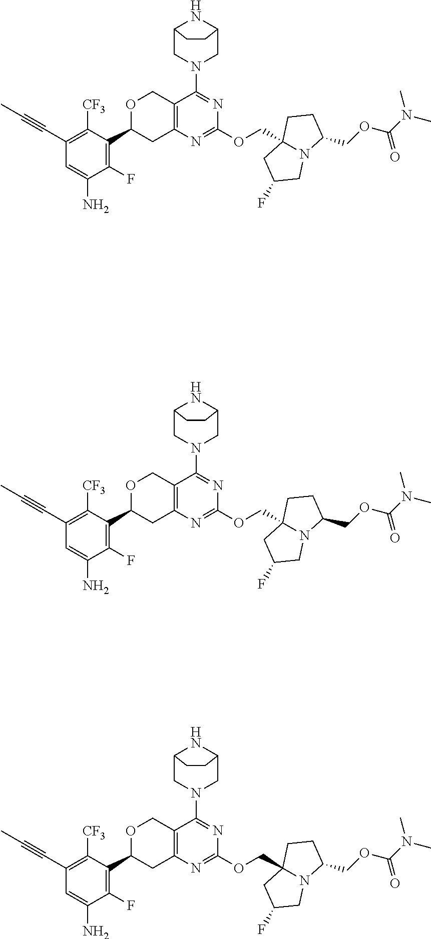

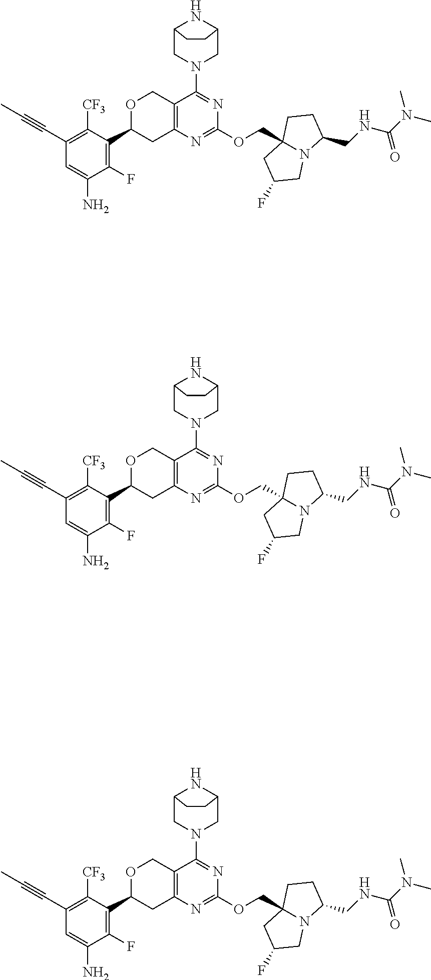

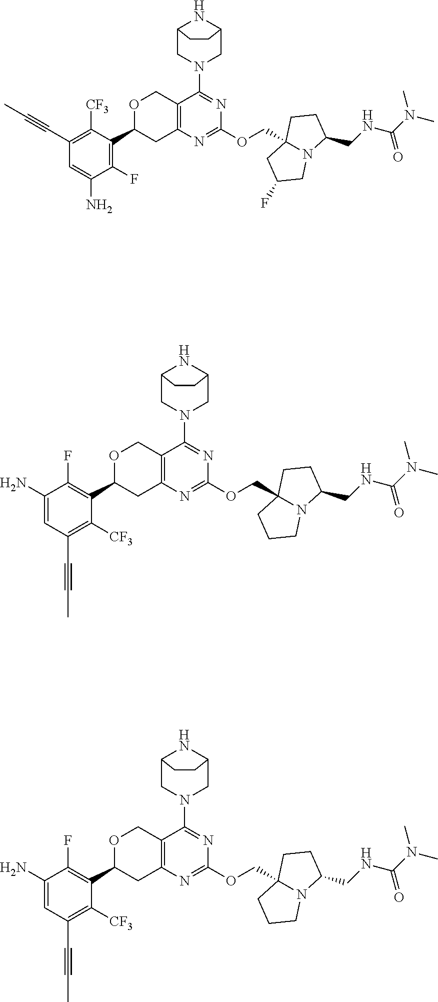 [SMILES: CC#Cc1cc(N)c(F)c([C@@H]2Cc3nc(OC[C@]45CC[C@@H](CNC(=O)N(C)C)N4C[C@H](F)C5)nc(N4CC5CCC(C4)N5)c3CO2)c1C(F)(F)F ; CC#Cc1cc(N)c(F)c([C@@H]2Cc3nc(OC[C@]45CCCN4[C@H](CNC(=O)N(C)C)CC5)nc(N4CC5CCC(C4)N5)c3CO2)c1C(F)(F)F ; CC#Cc1cc(N)c(F)c([C@@H]2Cc3nc(OC[C@@]45CCCN4[C@@H](CNC(=O)N(C)C)CC5)nc(N4CC5CCC(C4)N5)c3CO2)c1C(F)(F)F]

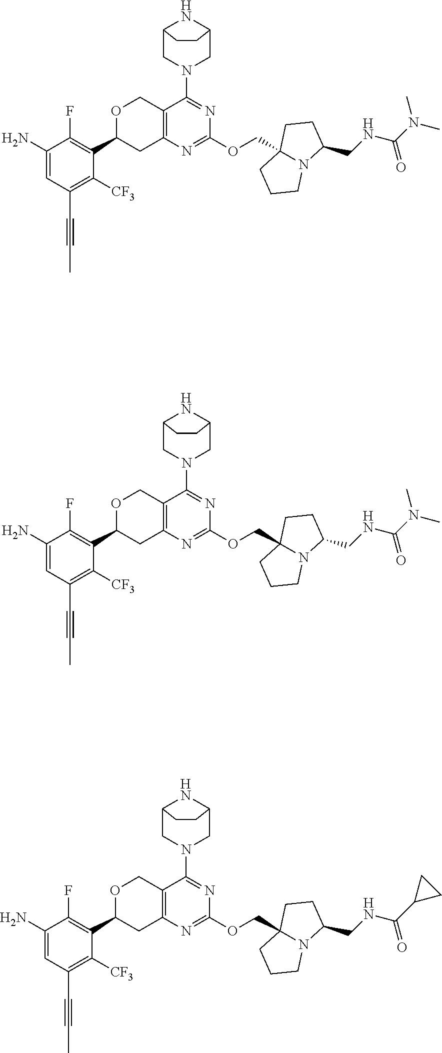 [SMILES: CC#Cc1cc(N)c(F)c([C@@H]2Cc3nc(OC[C@@]45CCCN4[C@H](CNC(=O)N(C)C)CC5)nc(N4CC5CCC(C4)N5)c3CO2)c1C(F)(F)F ; CC#Cc1cc(N)c(F)c([C@@H]2Cc3nc(OC[C@]45CCCN4[C@@H](CNC(=O)N(C)C)CC5)nc(N4CC5CCC(C4)N5)c3CO2)c1C(F)(F)F ; CC#Cc1cc(N)c(F)c([C@@H]2Cc3nc(OC[C@]45CCCN4[C@H](CNC(=O)C4CC4)CC5)nc(N4CC5CCC(C4)N5)c3CO2)c1C(F)(F)F]

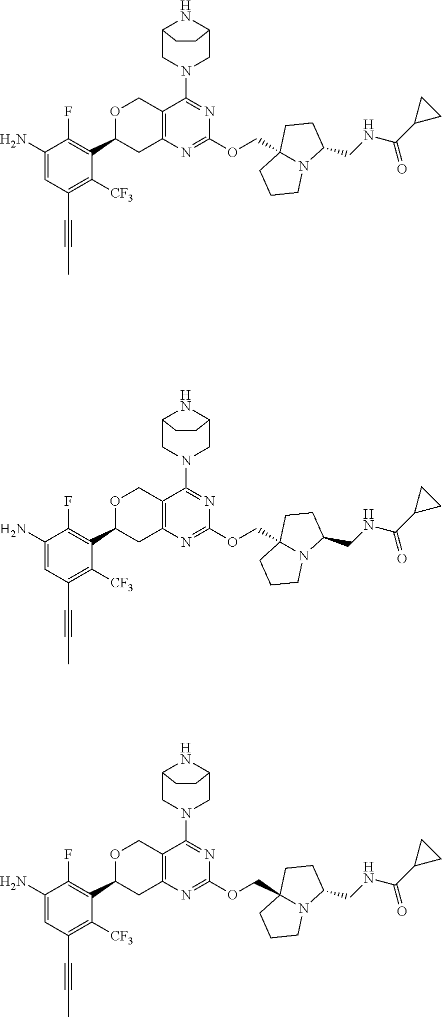 [SMILES: CC#Cc1cc(N)c(F)c([C@@H]2Cc3nc(OC[C@@]45CCCN4[C@@H](CNC(=O)C4CC4)CC5)nc(N4CC5CCC(C4)N5)c3CO2)c1C(F)(F)F ; CC#Cc1cc(N)c(F)c([C@@H]2Cc3nc(OC[C@@]45CCCN4[C@H](CNC(=O)C4CC4)CC5)nc(N4CC5CCC(C4)N5)c3CO2)c1C(F)(F)F ; CC#Cc1cc(N)c(F)c([C@@H]2Cc3nc(OC[C@]45CCCN4[C@@H](CNC(=O)C4CC4)CC5)nc(N4CC5CCC(C4)N5)c3CO2)c1C(F)(F)F]

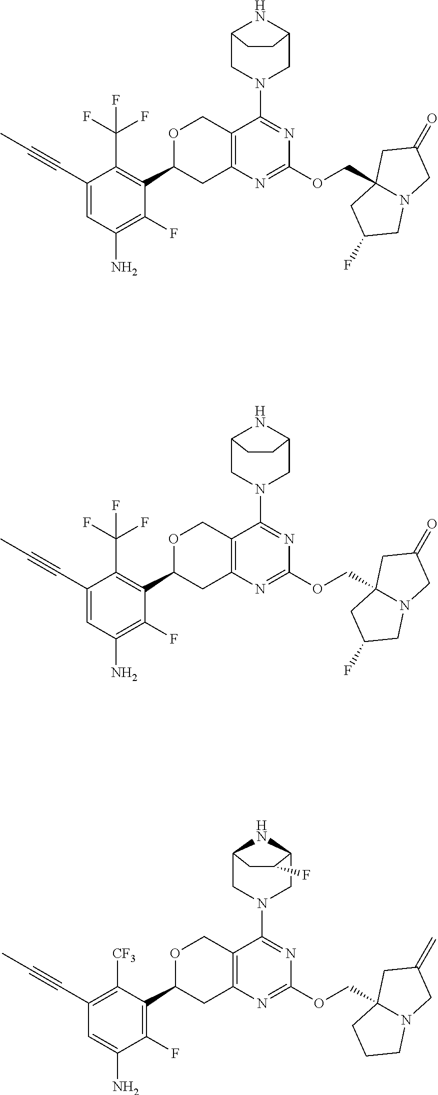 [SMILES: CC#Cc1cc(N)c(F)c([C@@H]2Cc3nc(OC[C@@]45CC(=O)CN4C[C@H](F)C5)nc(N4CC5CCC(C4)N5)c3CO2)c1C(F)(F)F ; CC#Cc1cc(N)c(F)c([C@@H]2Cc3nc(OC[C@]45CC(=O)CN4C[C@H](F)C5)nc(N4CC5CCC(C4)N5)c3CO2)c1C(F)(F)F ; C=C1CN2CCC[C@@]2(COc2nc3c(c(N4C[C@@H]5C[C@@H](F)[C@H](C4)N5)n2)CO[C@H](c2c(F)c(N)cc(C#CC)c2C(F)(F)F)C3)C1]

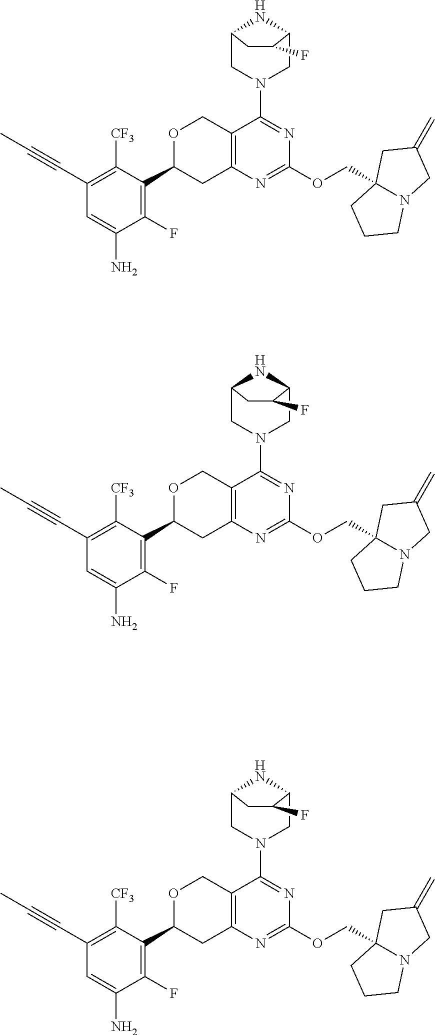

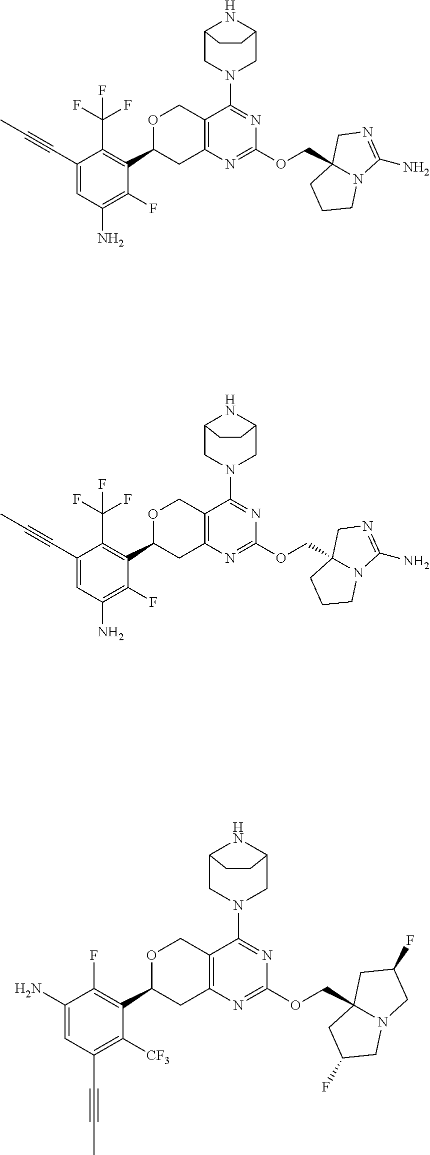

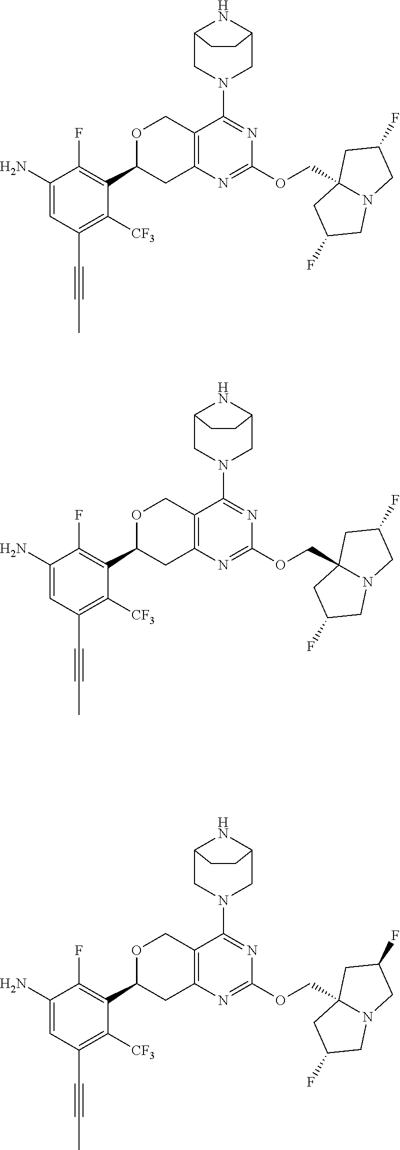

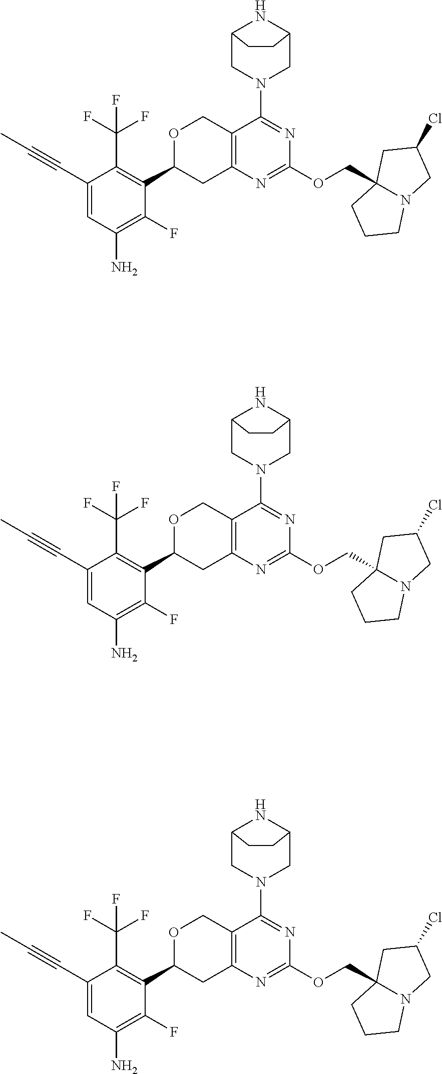 [SMILES: CC#Cc1cc(N)c(F)c([C@@H]2Cc3nc(OC[C@]45CCCN4C[C@H](Cl)C5)nc(N4CC5CCC(C4)N5)c3CO2)c1C(F)(F)F ; CC#Cc1cc(N)c(F)c([C@@H]2Cc3nc(OC[C@@]45CCCN4C[C@@H](Cl)C5)nc(N4CC5CCC(C4)N5)c3CO2)c1C(F)(F)F ; CC#Cc1cc(N)c(F)c([C@@H]2Cc3nc(OC[C@]45CCCN4C[C@@H](Cl)C5)nc(N4CC5CCC(C4)N5)c3CO2)c1C(F)(F)F]

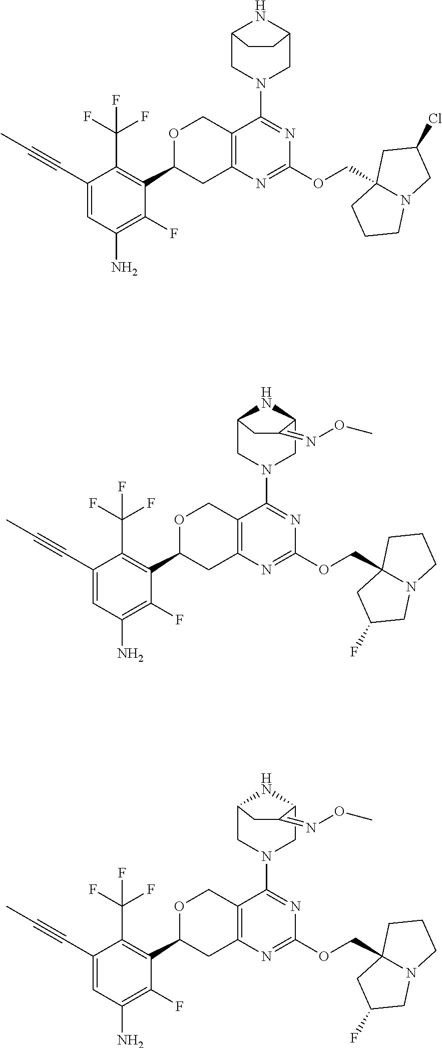 [SMILES: CC#Cc1cc(N)c(F)c([C@@H]2Cc3nc(OC[C@@]45CCCN4C[C@H](Cl)C5)nc(N4CC5CCC(C4)N5)c3CO2)c1C(F)(F)F ; CC#Cc1cc(N)c(F)c([C@@H]2Cc3nc(OC[C@@]45CCCN4C[C@H](F)C5)nc(N4C[C@@H]5C/C(=N/OC)[C@H](C4)N5)c3CO2)c1C(F)(F)F ; CC#Cc1cc(N)c(F)c([C@@H]2Cc3nc(OC[C@@]45CCCN4C[C@H](F)C5)nc(N4C[C@H]5C/C(=N/OC)[C@@H](C4)N5)c3CO2)c1C(F)(F)F]

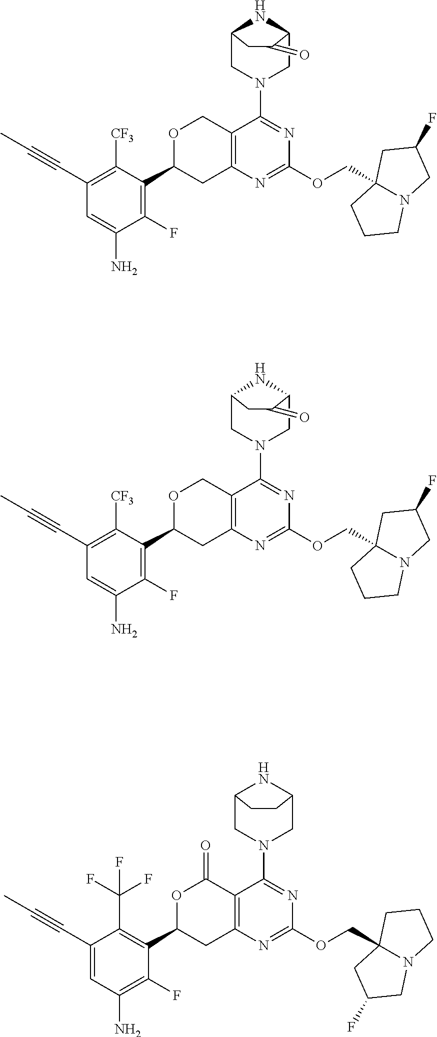 [SMILES: CC#Cc1cc(N)c(F)c([C@@H]2Cc3nc(OC[C@@]45CCCN4C[C@H](F)C5)nc(N4C[C@@H]5CC(=O)[C@H](C4)N5)c3CO2)c1C(F)(F)F ; CC#Cc1cc(N)c(F)c([C@@H]2Cc3nc(OC[C@@]45CCCN4C[C@H](F)C5)nc(N4C[C@H]5CC(=O)[C@@H](C4)N5)c3CO2)c1C(F)(F)F ; CC#Cc1cc(N)c(F)c([C@@H]2Cc3nc(OC[C@@]45CCCN4C[C@H](F)C5)nc(N4CC5CCC(C4)N5)c3C(=O)O2)c1C(F)(F)F]

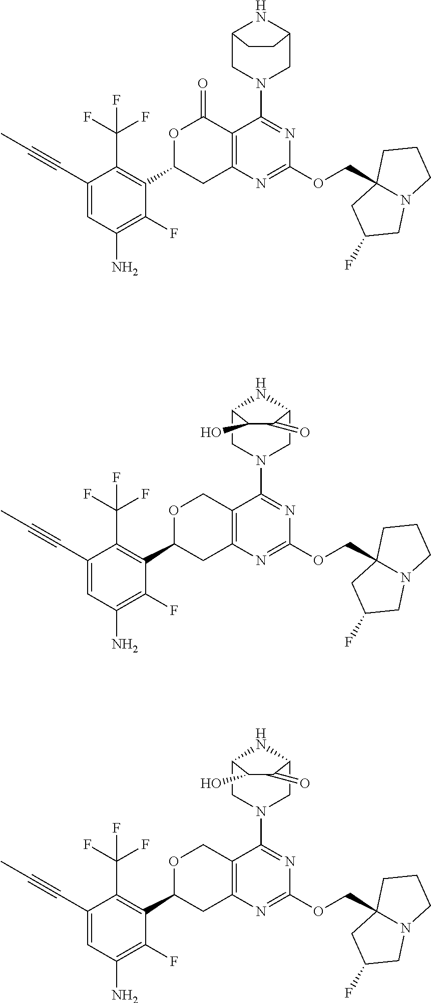 [SMILES: CC#Cc1cc(N)c(F)c([C@H]2Cc3nc(OC[C@@]45CCCN4C[C@H](F)C5)nc(N4CC5CCC(C4)N5)c3C(=O)O2)c1C(F)(F)F ; CC#Cc1cc(N)c(F)c([C@@H]2Cc3nc(OC[C@@]45CCCN4C[C@H](F)C5)nc(N4C[C@@H]5N[C@H](C4)C(=O)[C@H]5O)c3CO2)c1C(F)(F)F ; CC#Cc1cc(N)c(F)c([C@@H]2Cc3nc(OC[C@@]45CCCN4C[C@H](F)C5)nc(N4C[C@@H]5N[C@H](C4)C(=O)[C@@H]5O)c3CO2)c1C(F)(F)F]

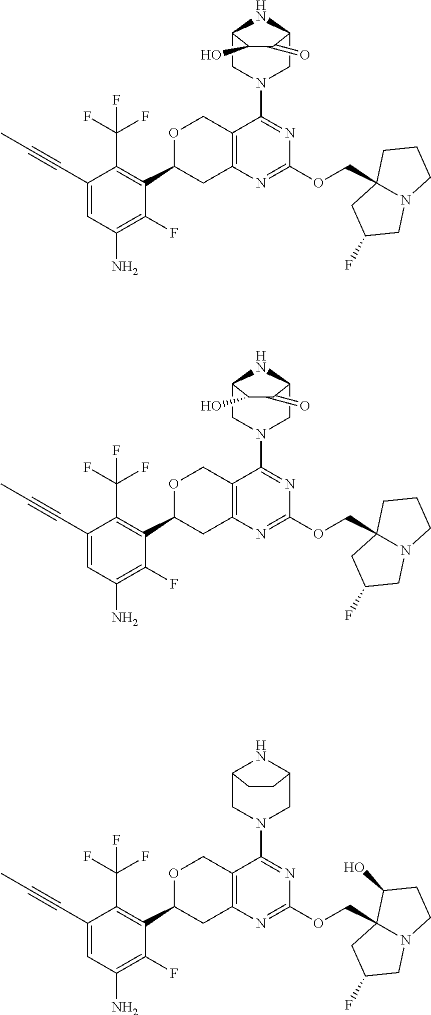 [SMILES: CC#Cc1cc(N)c(F)c([C@@H]2Cc3nc(OC[C@@]45CCCN4C[C@H](F)C5)nc(N4C[C@@H]5N[C@H](C4)[C@H](O)C5=O)c3CO2)c1C(F)(F)F ; CC#Cc1cc(N)c(F)c([C@@H]2Cc3nc(OC[C@@]45CCCN4C[C@H](F)C5)nc(N4C[C@@H]5N[C@H](C4)[C@@H](O)C5=O)c3CO2)c1C(F)(F)F ; CC#Cc1cc(N)c(F)c([C@@H]2Cc3nc(OC[C@]45C[C@@H](F)CN4CC[C@@H]5O)nc(N4CC5CCC(C4)N5)c3CO2)c1C(F)(F)F]

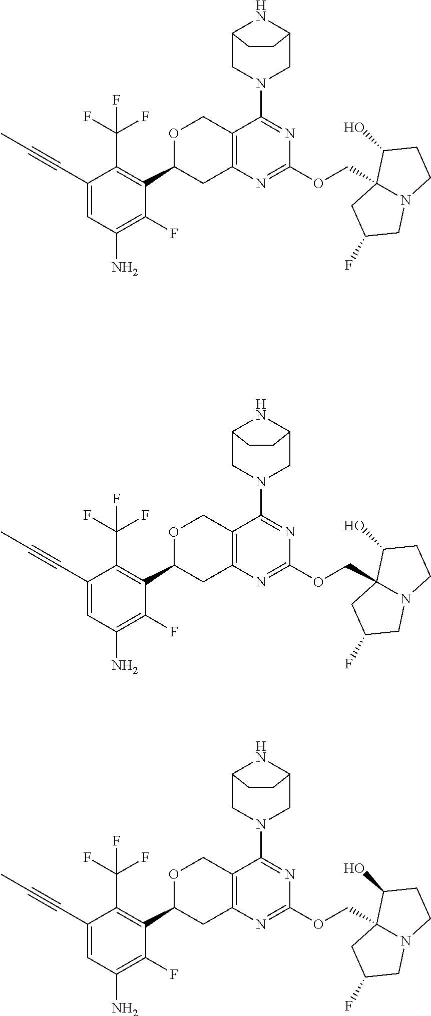 [SMILES: CC#Cc1cc(N)c(F)c([C@@H]2Cc3nc(OC[C@@]45C[C@@H](F)CN4CC[C@H]5O)nc(N4CC5CCC(C4)N5)c3CO2)c1C(F)(F)F ; CC#Cc1cc(N)c(F)c([C@@H]2Cc3nc(OC[C@]45C[C@@H](F)CN4CC[C@H]5O)nc(N4CC5CCC(C4)N5)c3CO2)c1C(F)(F)F ; CC#Cc1cc(N)c(F)c([C@@H]2Cc3nc(OC[C@@]45C[C@@H](F)CN4CC[C@@H]5O)nc(N4CC5CCC(C4)N5)c3CO2)c1C(F)(F)F]

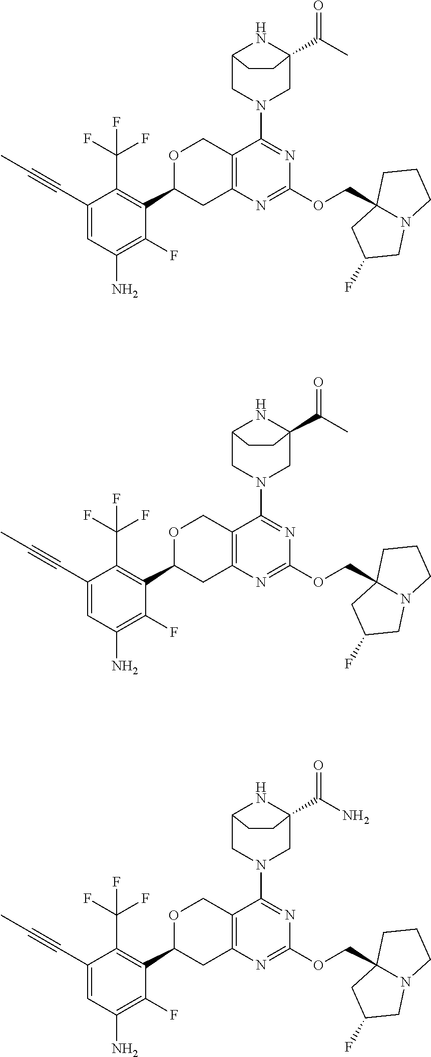 [SMILES: CC#Cc1cc(N)c(F)c([C@@H]2Cc3nc(OC[C@@]45CCCN4C[C@H](F)C5)nc(N4CC5CC[C@](C(C)=O)(C4)N5)c3CO2)c1C(F)(F)F ; CC#Cc1cc(N)c(F)c([C@@H]2Cc3nc(OC[C@@]45CCCN4C[C@H](F)C5)nc(N4CC5CC[C@@](C(C)=O)(C4)N5)c3CO2)c1C(F)(F)F ; CC#Cc1cc(N)c(F)c([C@@H]2Cc3nc(OC[C@@]45CCCN4C[C@H](F)C5)nc(N4CC5CC[C@](C(N)=O)(C4)N5)c3CO2)c1C(F)(F)F]

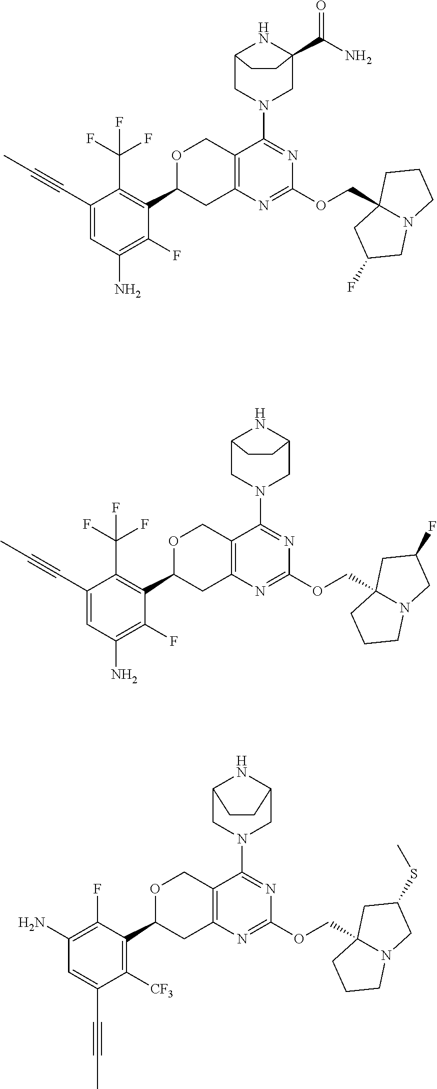 [SMILES: CC#Cc1cc(N)c(F)c([C@@H]2Cc3nc(OC[C@@]45CCCN4C[C@H](F)C5)nc(N4CC5CC[C@@](C(N)=O)(C4)N5)c3CO2)c1C(F)(F)F ; CC#Cc1cc(N)c(F)c([C@@H]2Cc3nc(OC[C@@]45CCCN4C[C@H](F)C5)nc(N4CC5CCC(C4)N5)c3CO2)c1C(F)(F)F ; CC#Cc1cc(N)c(F)c([C@@H]2Cc3nc(OC[C@@]45CCCN4C[C@@H](SC)C5)nc(N4CC5CCC(C4)N5)c3CO2)c1C(F)(F)F]

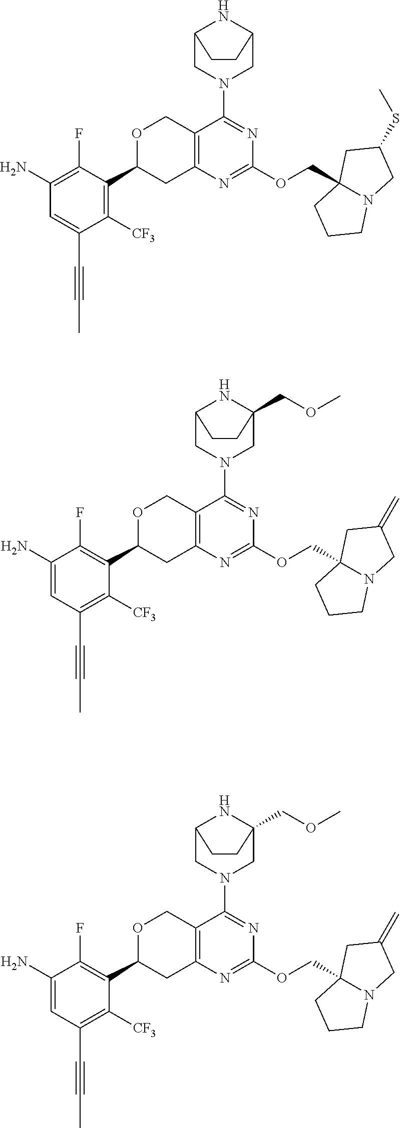 [SMILES: CC#Cc1cc(N)c(F)c([C@@H]2Cc3nc(OC[C@]45CCCN4C[C@@H](SC)C5)nc(N4CC5CCC(C4)N5)c3CO2)c1C(F)(F)F ; C=C1CN2CCC[C@@]2(COc2nc3c(c(N4CC5CC[C@@](COC)(C4)N5)n2)CO[C@H](c2c(F)c(N)cc(C#CC)c2C(F)(F)F)C3)C1 ; C=C1CN2CCC[C@@]2(COc2nc3c(c(N4CC5CC[C@](COC)(C4)N5)n2)CO[C@H](c2c(F)c(N)cc(C#CC)c2C(F)(F)F)C3)C1]

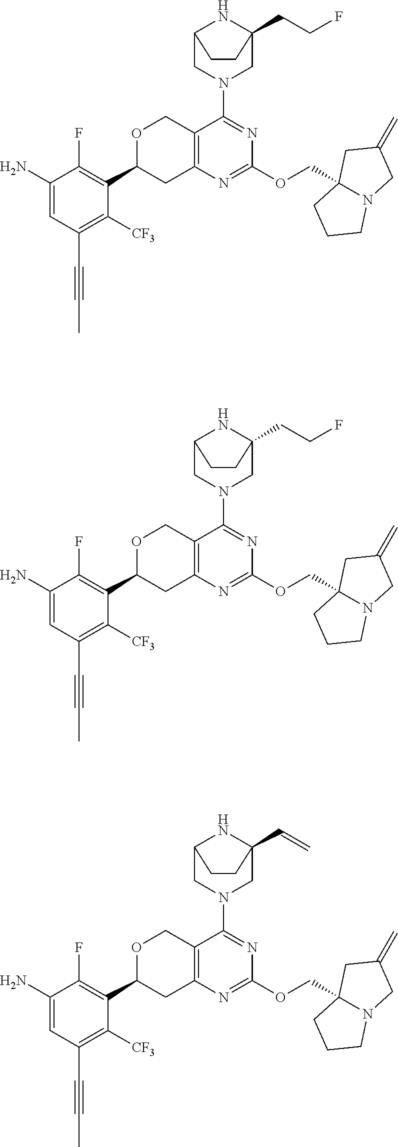 [SMILES: C=C1CN2CCC[C@@]2(COc2nc3c(c(N4CC5CC[C@@](CCF)(C4)N5)n2)CO[C@H](c2c(F)c(N)cc(C#CC)c2C(F)(F)F)C3)C1 ; C=C1CN2CCC[C@@]2(COc2nc3c(c(N4CC5CC[C@](CCF)(C4)N5)n2)CO[C@H](c2c(F)c(N)cc(C#CC)c2C(F)(F)F)C3)C1 ; C=C[C@]12CCC(CN(c3nc(OC[C@@]45CCCN4CC(=C)C5)nc4c3CO[C@H](c3c(F)c(N)cc(C#CC)c3C(F)(F)F)C4)C1)N2]

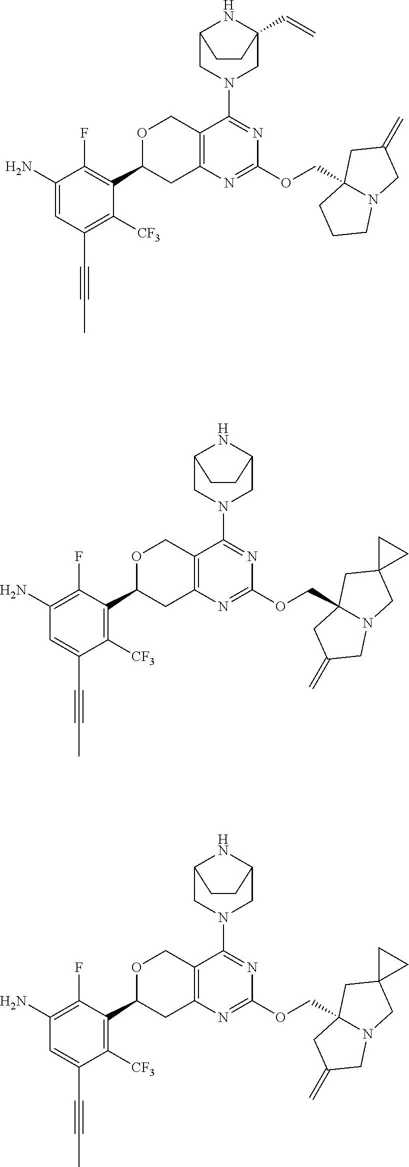 [SMILES: C=C[C@@]12CCC(CN(c3nc(OC[C@@]45CCCN4CC(=C)C5)nc4c3CO[C@H](c3c(F)c(N)cc(C#CC)c3C(F)(F)F)C4)C1)N2 ; C=C1CN2CC3(CC3)C[C@@]2(COc2nc3c(c(N4CC5CCC(C4)N5)n2)CO[C@H](c2c(F)c(N)cc(C#CC)c2C(F)(F)F)C3)C1 ; C=C1CN2CC3(CC3)C[C@]2(COc2nc3c(c(N4CC5CCC(C4)N5)n2)CO[C@H](c2c(F)c(N)cc(C#CC)c2C(F)(F)F)C3)C1]

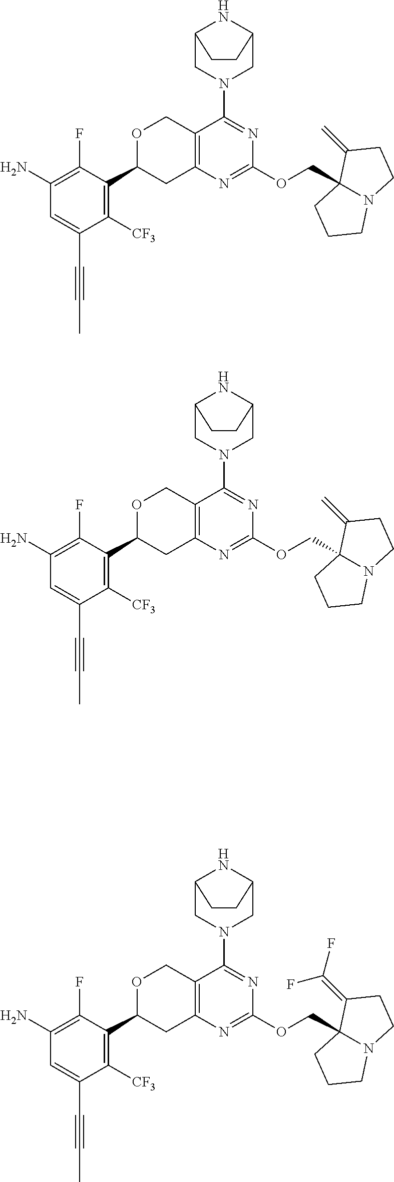 [SMILES: C=C1CCN2CCC[C@]12COc1nc2c(c(N3CC4CCC(C3)N4)n1)CO[C@H](c1c(F)c(N)cc(C#CC)c1C(F)(F)F)C2 ; C=C1CCN2CCC[C@@]12COc1nc2c(c(N3CC4CCC(C3)N4)n1)CO[C@H](c1c(F)c(N)cc(C#CC)c1C(F)(F)F)C2 ; CC#Cc1cc(N)c(F)c([C@@H]2Cc3nc(OC[C@]45CCCN4CCC5=C(F)F)nc(N4CC5CCC(C4)N5)c3CO2)c1C(F)(F)F]

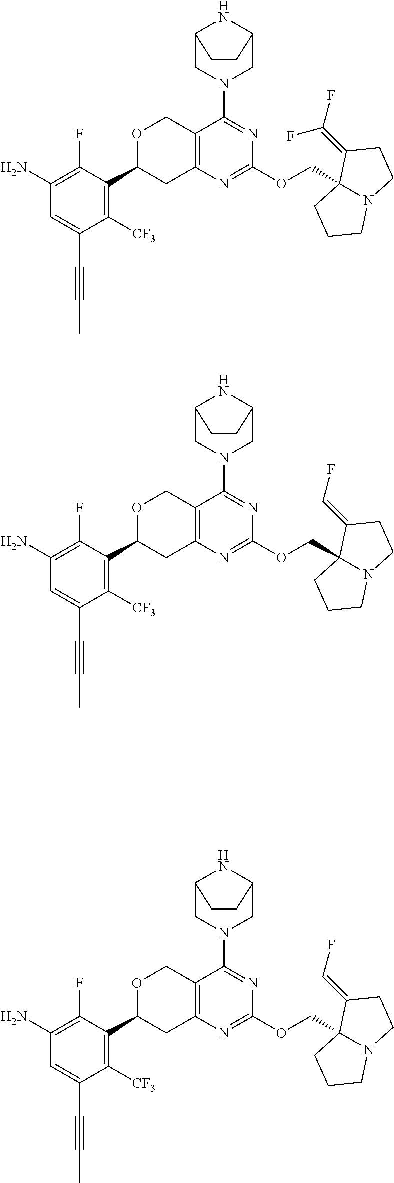 [SMILES: CC#Cc1cc(N)c(F)c([C@@H]2Cc3nc(OC[C@@]45CCCN4CCC5=C(F)F)nc(N4CC5CCC(C4)N5)c3CO2)c1C(F)(F)F ; CC#Cc1cc(N)c(F)c([C@@H]2Cc3nc(OC[C@]45CCCN4CC/C5=C\F)nc(N4CC5CCC(C4)N5)c3CO2)c1C(F)(F)F ; CC#Cc1cc(N)c(F)c([C@@H]2Cc3nc(OC[C@@]45CCCN4CC/C5=C\F)nc(N4CC5CCC(C4)N5)c3CO2)c1C(F)(F)F]

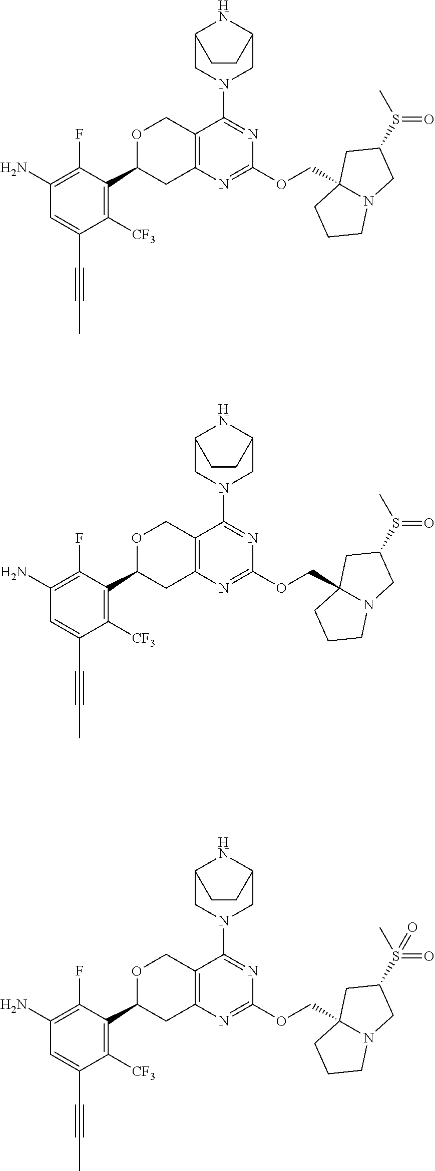 [SMILES: CC#Cc1cc(N)c(F)c([C@@H]2Cc3nc(OC[C@@]45CCCN4C[C@@H](S(C)=O)C5)nc(N4CC5CCC(C4)N5)c3CO2)c1C(F)(F)F ; CC#Cc1cc(N)c(F)c([C@@H]2Cc3nc(OC[C@]45CCCN4C[C@@H](S(C)=O)C5)nc(N4CC5CCC(C4)N5)c3CO2)c1C(F)(F)F ; CC#Cc1cc(N)c(F)c([C@@H]2Cc3nc(OC[C@@]45CCCN4C[C@@H](S(C)(=O)=O)C5)nc(N4CC5CCC(C4)N5)c3CO2)c1C(F)(F)F]

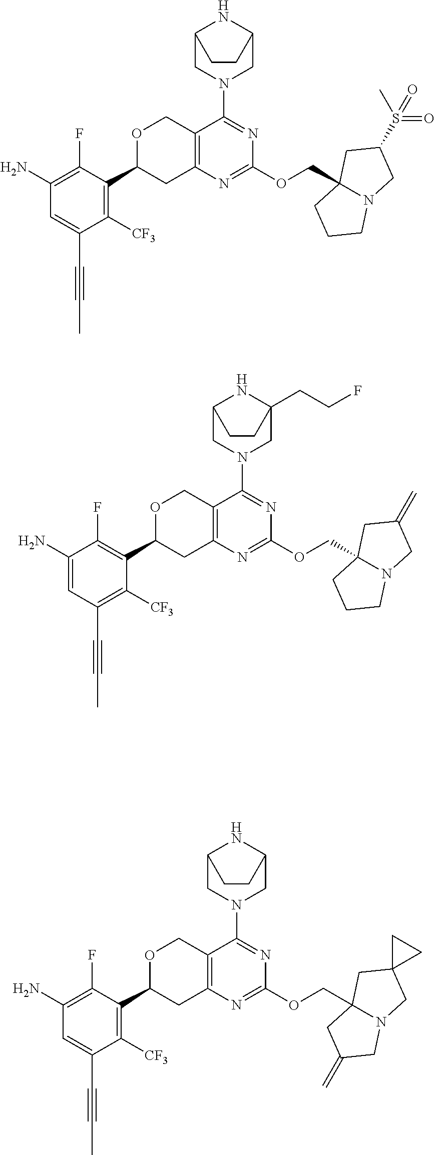 [SMILES: CC#Cc1cc(N)c(F)c([C@@H]2Cc3nc(OC[C@]45CCCN4C[C@@H](S(C)(=O)=O)C5)nc(N4CC5CCC(C4)N5)c3CO2)c1C(F)(F)F ; C=C1CN2CCC[C@@]2(COc2nc3c(c(N4CC5CCC(CCF)(C4)N5)n2)CO[C@H](c2c(F)c(N)cc(C#CC)c2C(F)(F)F)C3)C1 ; C=C1CN2CC3(CC3)CC2(COc2nc3c(c(N4CC5CCC(C4)N5)n2)CO[C@H](c2c(F)c(N)cc(C#CC)c2C(F)(F)F)C3)C1]

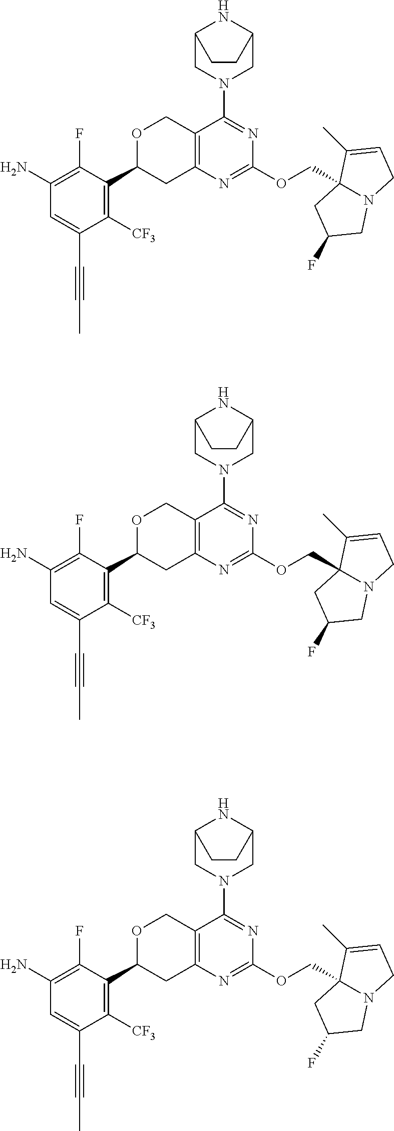 [SMILES: CC#Cc1cc(N)c(F)c([C@@H]2Cc3nc(OC[C@@]45C[C@H](F)CN4CC=C5C)nc(N4CC5CCC(C4)N5)c3CO2)c1C(F)(F)F ; CC#Cc1cc(N)c(F)c([C@@H]2Cc3nc(OC[C@]45C[C@H](F)CN4CC=C5C)nc(N4CC5CCC(C4)N5)c3CO2)c1C(F)(F)F ; CC#Cc1cc(N)c(F)c([C@@H]2Cc3nc(OC[C@@]45C[C@@H](F)CN4CC=C5C)nc(N4CC5CCC(C4)N5)c3CO2)c1C(F)(F)F]

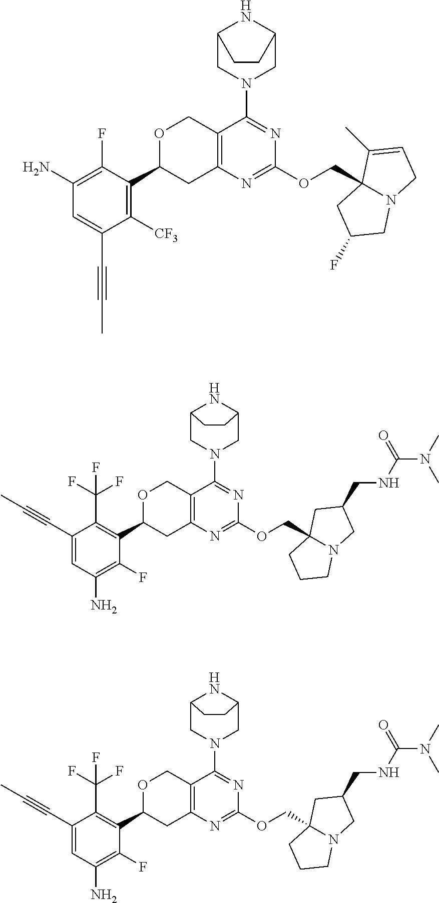

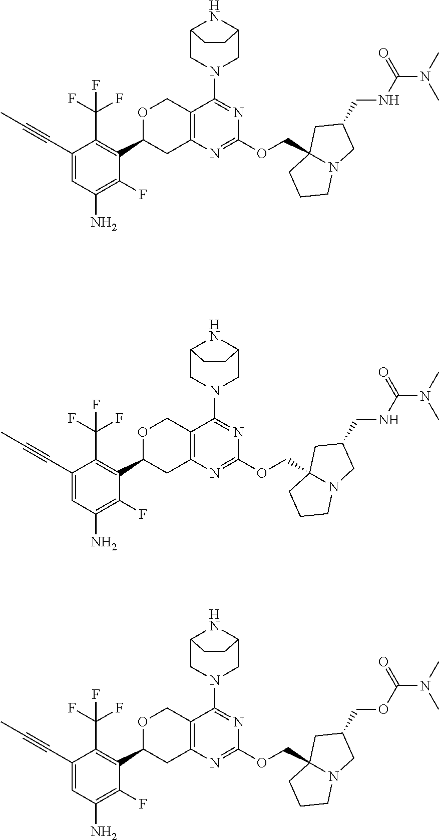

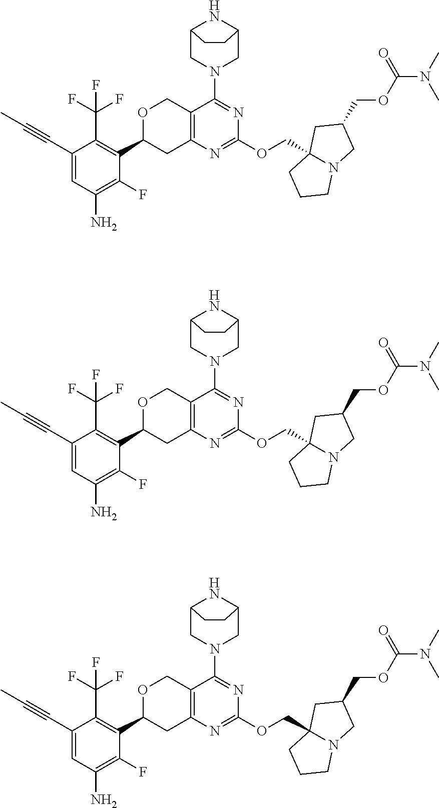

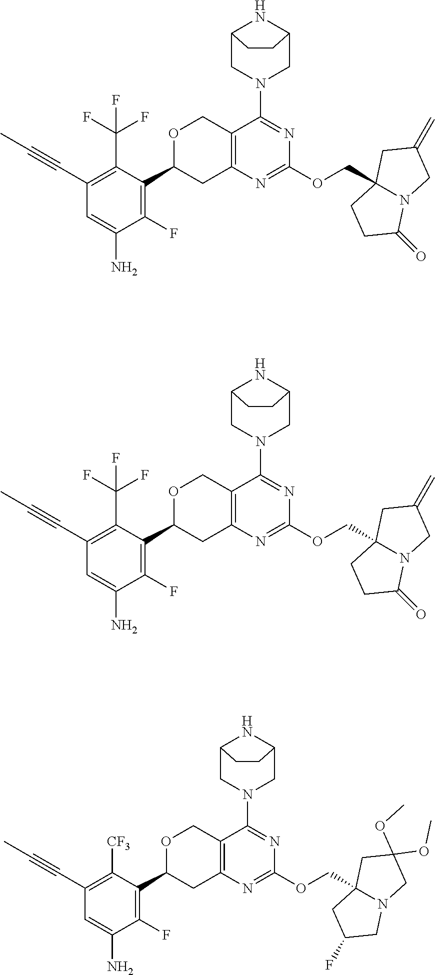

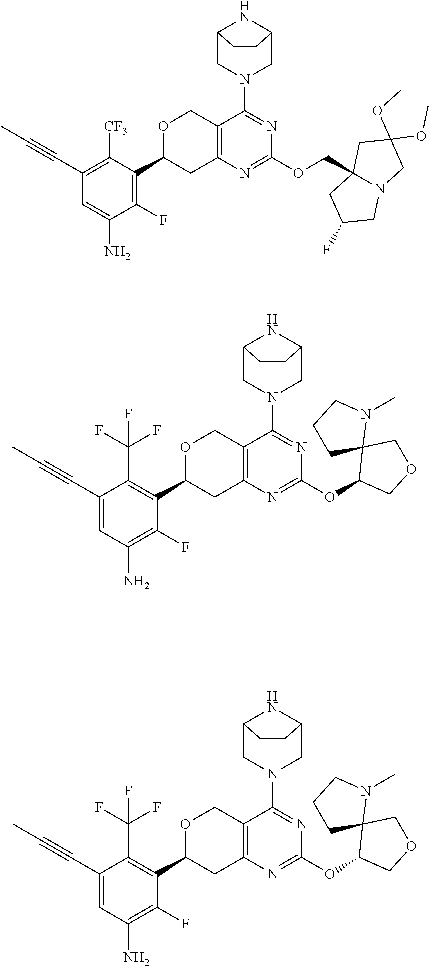

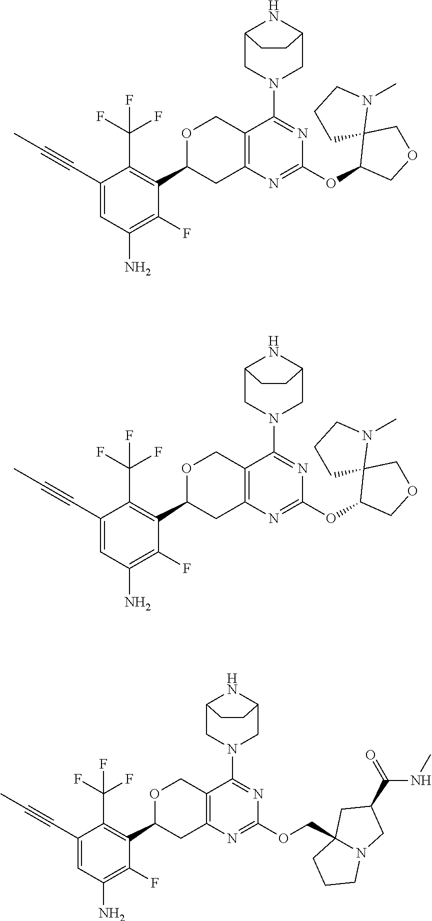 [SMILES: CC#Cc1cc(N)c(F)c([C@@H]2Cc3nc(O[C@@H]4COC[C@@]45CCCN5C)nc(N4CC5CCC(C4)N5)c3CO2)c1C(F)(F)F ; CC#Cc1cc(N)c(F)c([C@@H]2Cc3nc(O[C@H]4COC[C@@]45CCCN5C)nc(N4CC5CCC(C4)N5)c3CO2)c1C(F)(F)F ; CC#Cc1cc(N)c(F)c([C@@H]2Cc3nc(OC[C@]45CCCN4C[C@H](C(=O)NC)C5)nc(N4CC5CCC(C4)N5)c3CO2)c1C(F)(F)F]

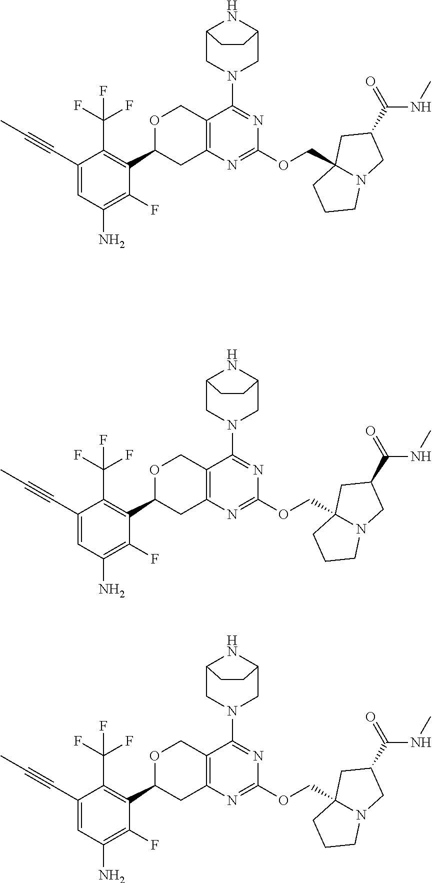

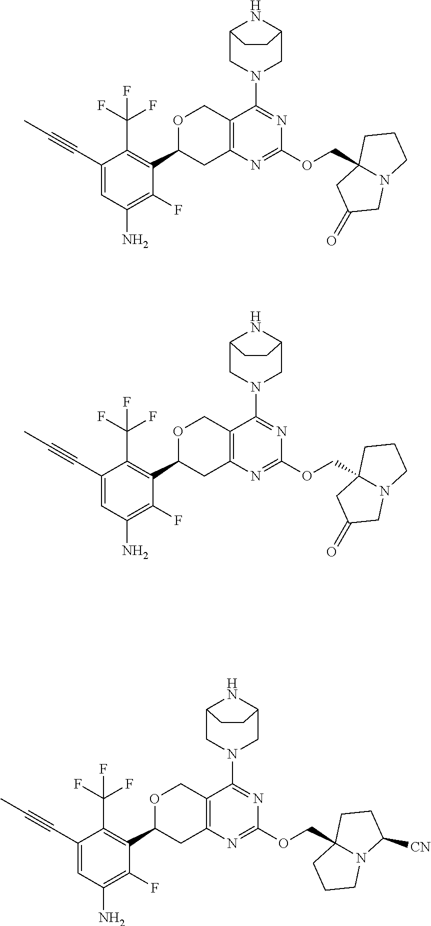 [SMILES: CC#Cc1cc(N)c(F)c([C@@H]2Cc3nc(OC[C@@]45CCCN4CC(=O)C5)nc(N4CC5CCC(C4)N5)c3CO2)c1C(F)(F)F ; CC#Cc1cc(N)c(F)c([C@@H]2Cc3nc(OC[C@]45CCCN4CC(=O)C5)nc(N4CC5CCC(C4)N5)c3CO2)c1C(F)(F)F ; CC#Cc1cc(N)c(F)c([C@@H]2Cc3nc(OC[C@]45CCCN4[C@H](C#N)CC5)nc(N4CC5CCC(C4)N5)c3CO2)c1C(F)(F)F]

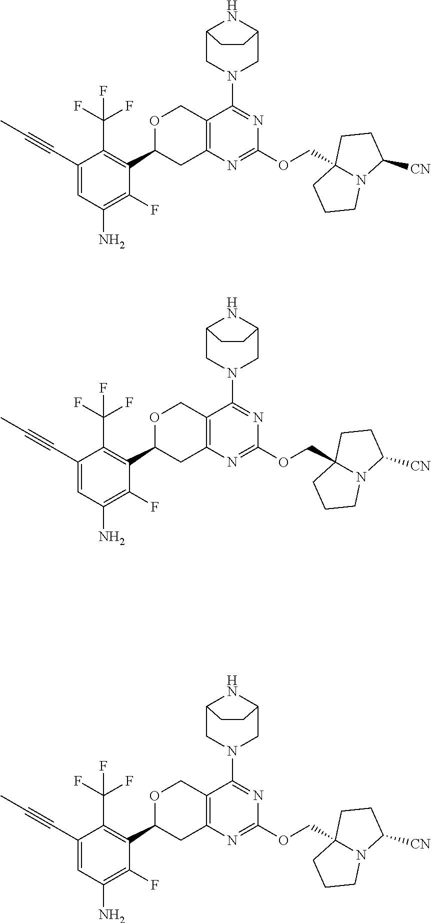 [SMILES: CC#Cc1cc(N)c(F)c([C@@H]2Cc3nc(OC[C@@]45CCCN4[C@H](C#N)CC5)nc(N4CC5CCC(C4)N5)c3CO2)c1C(F)(F)F ; CC#Cc1cc(N)c(F)c([C@@H]2Cc3nc(OC[C@]45CCCN4[C@@H](C#N)CC5)nc(N4CC5CCC(C4)N5)c3CO2)c1C(F)(F)F ; CC#Cc1cc(N)c(F)c([C@@H]2Cc3nc(OC[C@@]45CCCN4[C@@H](C#N)CC5)nc(N4CC5CCC(C4)N5)c3CO2)c1C(F)(F)F]

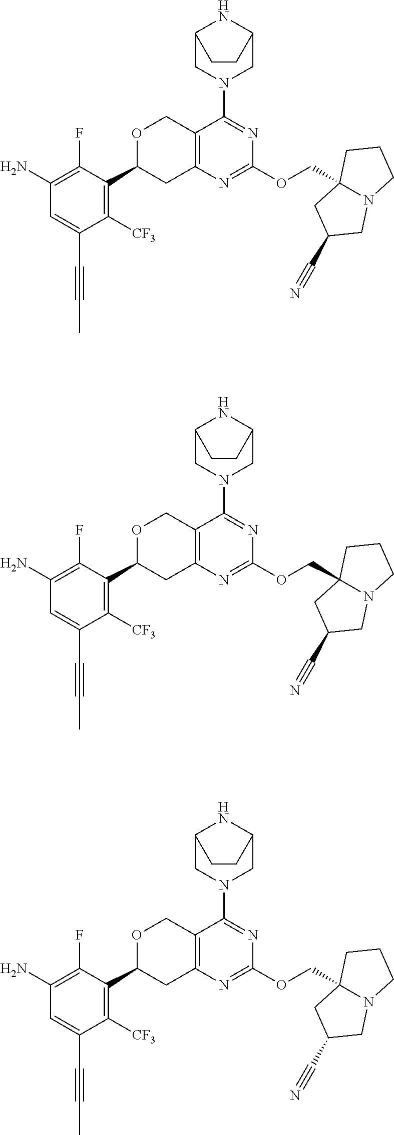 [SMILES: CC#Cc1cc(N)c(F)c([C@@H]2Cc3nc(OC[C@]45CCCN4C[C@@H](C#N)C5)nc(N4CC5CCC(C4)N5)c3CO2)c1C(F)(F)F ; CC#Cc1cc(N)c(F)c([C@@H]2Cc3nc(OC[C@@]45CCCN4C[C@@H](C#N)C5)nc(N4CC5CCC(C4)N5)c3CO2)c1C(F)(F)F ; CC#Cc1cc(N)c(F)c([C@@H]2Cc3nc(OC[C@]45CCCN4C[C@H](C#N)C5)nc(N4CC5CCC(C4)N5)c3CO2)c1C(F)(F)F]

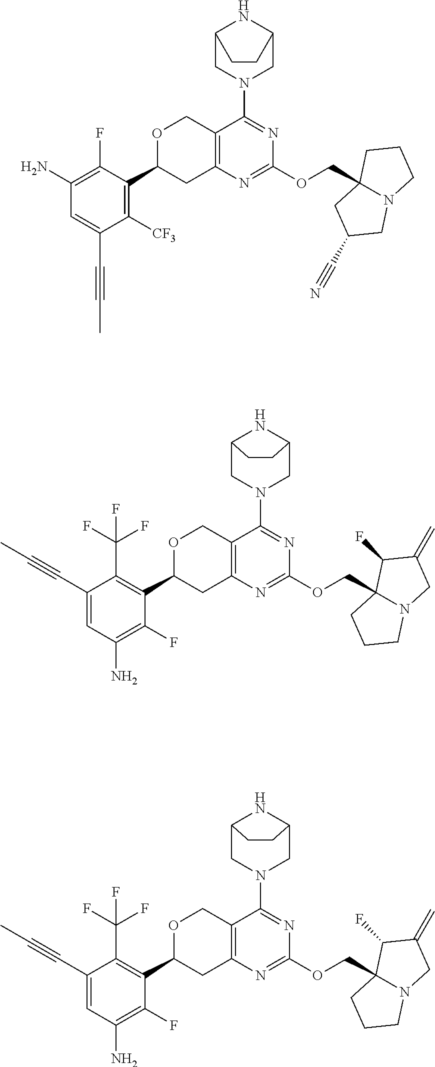

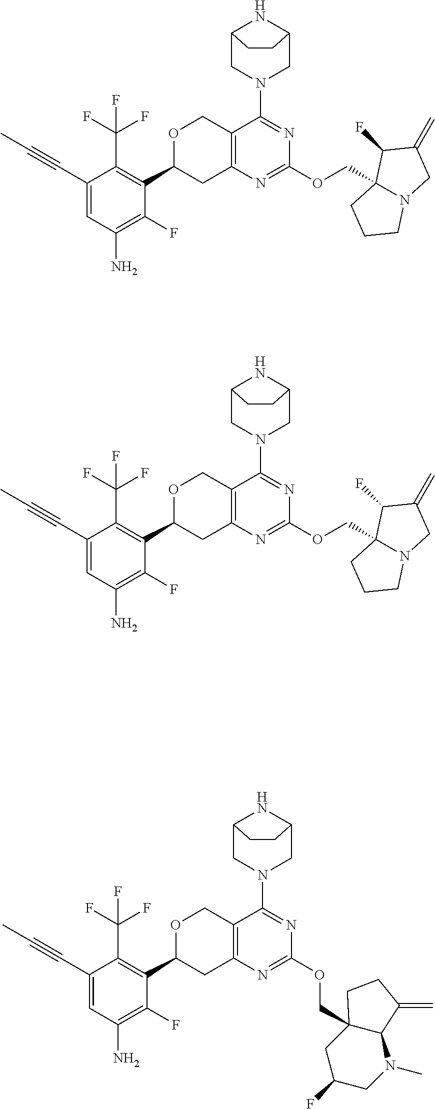

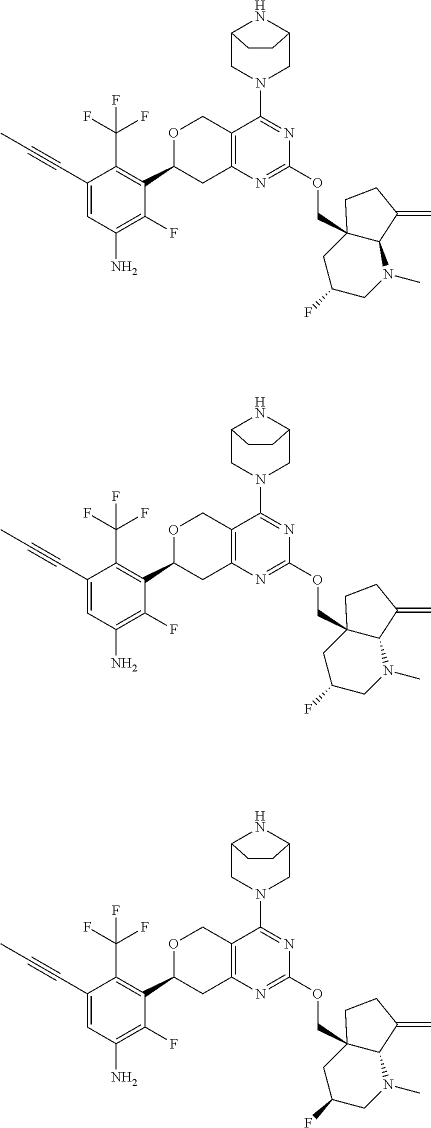

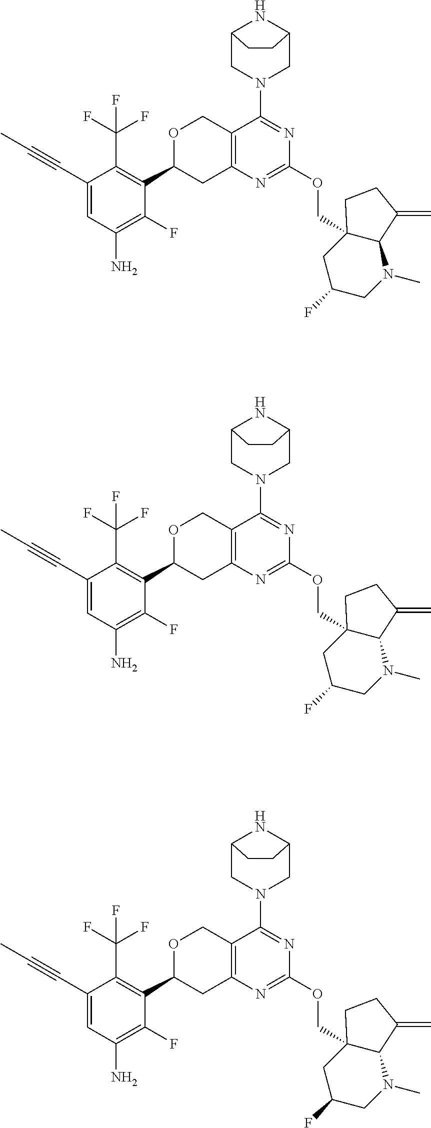

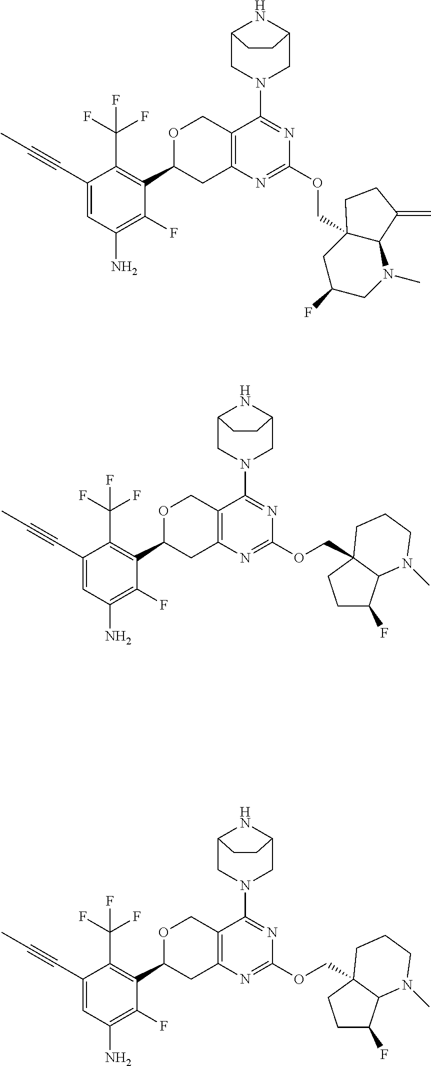

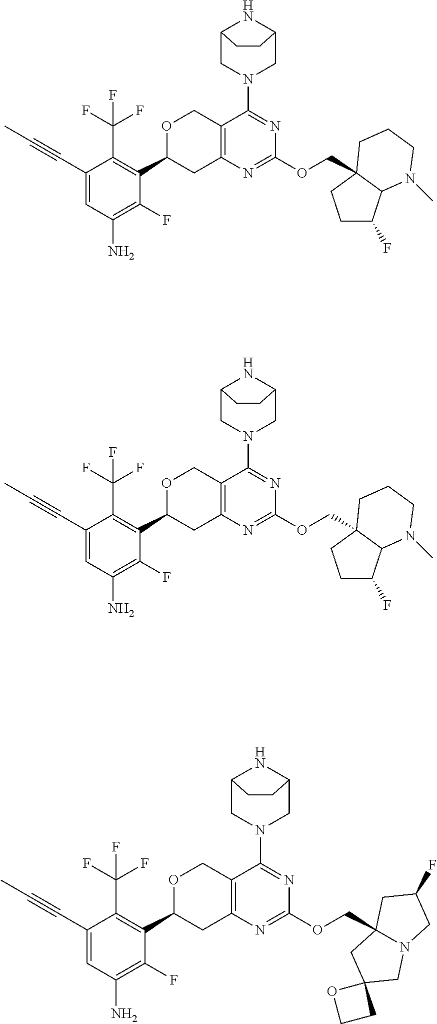

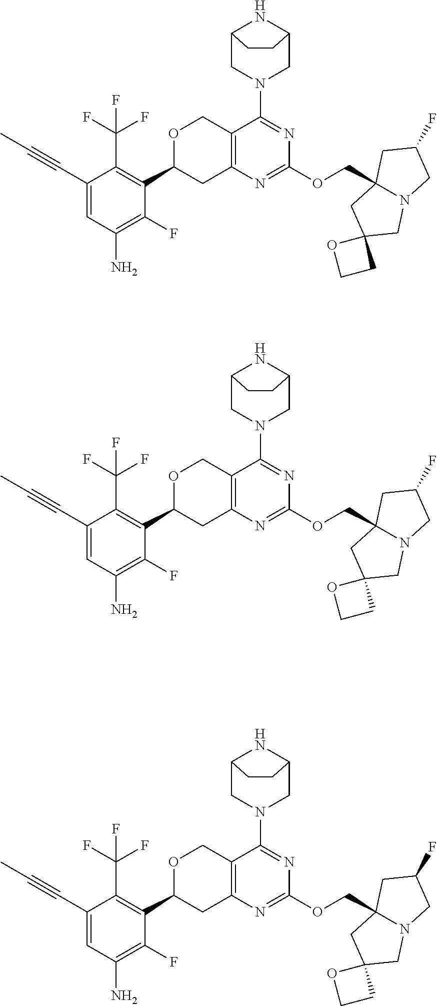 [SMILES: CC#Cc1cc(N)c(F)c([C@@H]2Cc3nc(OC[C@@]45C[C@H](F)CN4C[C@@]4(CCO4)C5)nc(N4CC5CCC(C4)N5)c3CO2)c1C(F)(F)F ; CC#Cc1cc(N)c(F)c([C@@H]2Cc3nc(OC[C@@]45C[C@H](F)CN4C[C@]4(CCO4)C5)nc(N4CC5CCC(C4)N5)c3CO2)c1C(F)(F)F ; CC#Cc1cc(N)c(F)c([C@@H]2Cc3nc(OC[C@@]45C[C@@H](F)CN4C[C@]4(CCO4)C5)nc(N4CC5CCC(C4)N5)c3CO2)c1C(F)(F)F]

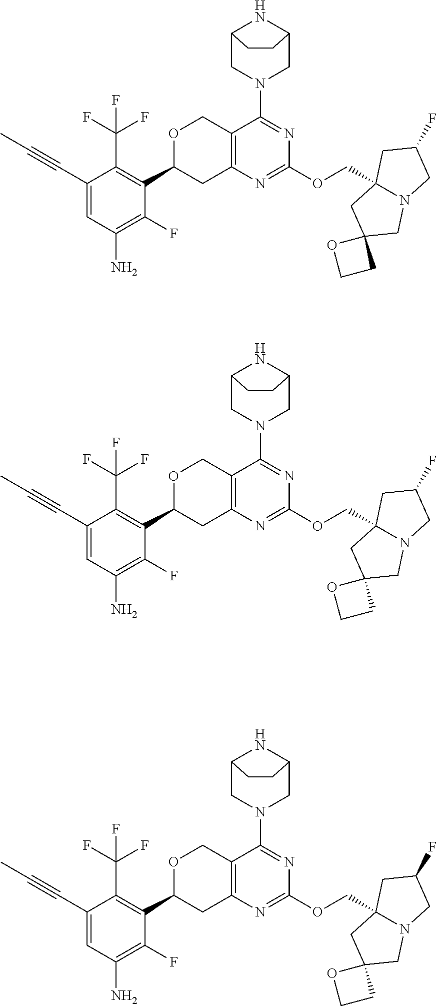 [SMILES: CC#Cc1cc(N)c(F)c([C@@H]2Cc3nc(OC[C@]45C[C@H](F)CN4C[C@@]4(CCO4)C5)nc(N4CC5CCC(C4)N5)c3CO2)c1C(F)(F)F ; CC#Cc1cc(N)c(F)c([C@@H]2Cc3nc(OC[C@]45C[C@H](F)CN4C[C@]4(CCO4)C5)nc(N4CC5CCC(C4)N5)c3CO2)c1C(F)(F)F ; CC#Cc1cc(N)c(F)c([C@@H]2Cc3nc(OC[C@]45C[C@@H](F)CN4C[C@]4(CCO4)C5)nc(N4CC5CCC(C4)N5)c3CO2)c1C(F)(F)F]

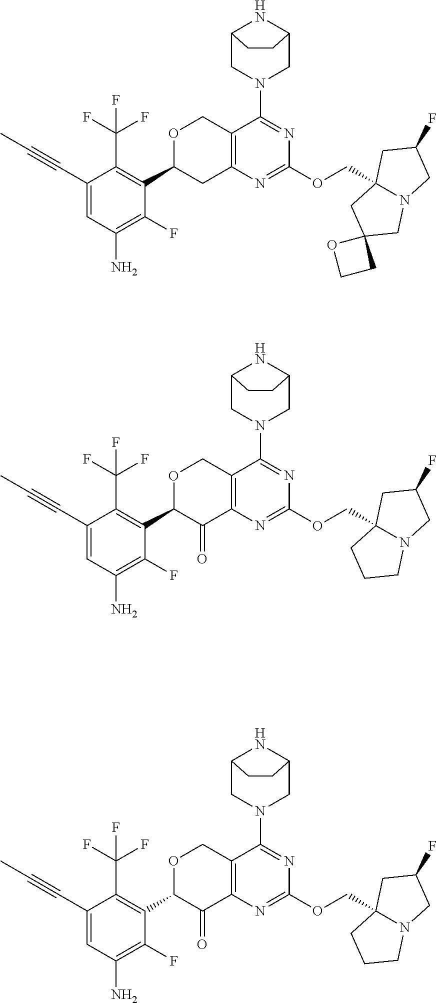 [SMILES: CC#Cc1cc(N)c(F)c([C@@H]2Cc3nc(OC[C@]45C[C@@H](F)CN4C[C@@]4(CCO4)C5)nc(N4CC5CCC(C4)N5)c3CO2)c1C(F)(F)F ; CC#Cc1cc(N)c(F)c([C@H]2OCc3c(nc(OC[C@@]45CCCN4C[C@H](F)C5)nc3N3CC4CCC(C3)N4)C2=O)c1C(F)(F)F ; CC#Cc1cc(N)c(F)c([C@@H]2OCc3c(nc(OC[C@@]45CCCN4C[C@H](F)C5)nc3N3CC4CCC(C3)N4)C2=O)c1C(F)(F)F]

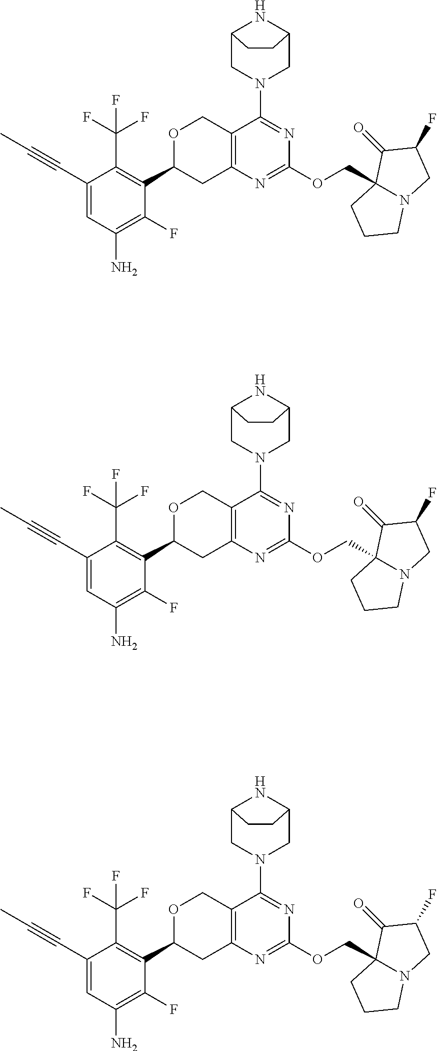 [SMILES: CC#Cc1cc(N)c(F)c([C@@H]2Cc3nc(OC[C@]45CCCN4C[C@H](F)C5=O)nc(N4CC5CCC(C4)N5)c3CO2)c1C(F)(F)F ; CC#Cc1cc(N)c(F)c([C@@H]2Cc3nc(OC[C@@]45CCCN4C[C@H](F)C5=O)nc(N4CC5CCC(C4)N5)c3CO2)c1C(F)(F)F ; CC#Cc1cc(N)c(F)c([C@@H]2Cc3nc(OC[C@]45CCCN4C[C@@H](F)C5=O)nc(N4CC5CCC(C4)N5)c3CO2)c1C(F)(F)F]

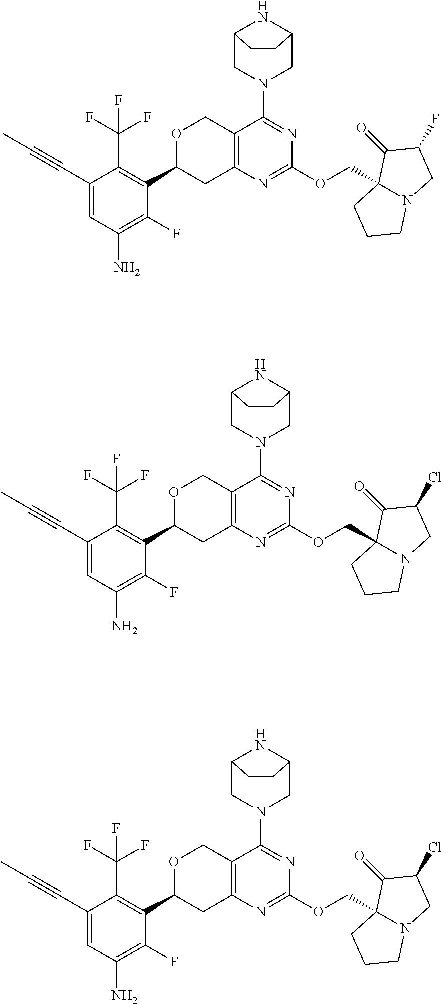 [SMILES: CC#Cc1cc(N)c(F)c([C@@H]2Cc3nc(OC[C@@]45CCCN4C[C@@H](F)C5=O)nc(N4CC5CCC(C4)N5)c3CO2)c1C(F)(F)F ; CC#Cc1cc(N)c(F)c([C@@H]2Cc3nc(OC[C@]45CCCN4C[C@H](Cl)C5=O)nc(N4CC5CCC(C4)N5)c3CO2)c1C(F)(F)F ; CC#Cc1cc(N)c(F)c([C@@H]2Cc3nc(OC[C@@]45CCCN4C[C@H](Cl)C5=O)nc(N4CC5CCC(C4)N5)c3CO2)c1C(F)(F)F]

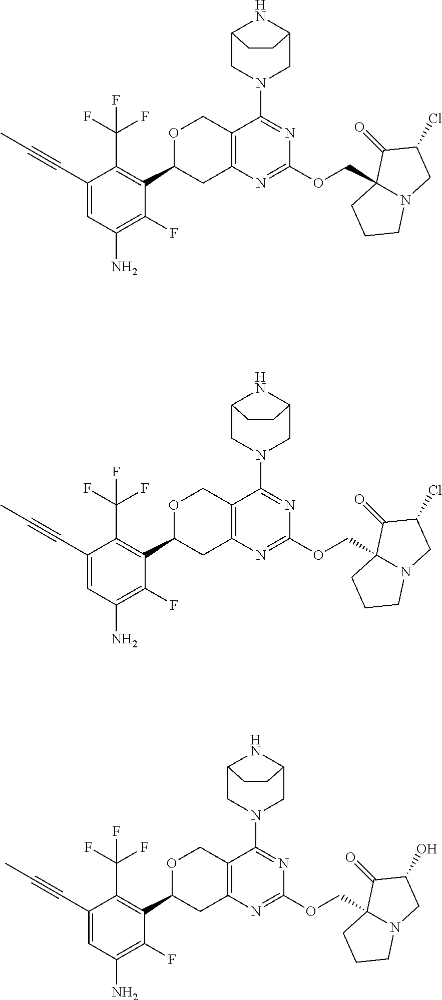 [SMILES: CC#Cc1cc(N)c(F)c([C@@H]2Cc3nc(OC[C@]45CCCN4C[C@@H](Cl)C5=O)nc(N4CC5CCC(C4)N5)c3CO2)c1C(F)(F)F ; CC#Cc1cc(N)c(F)c([C@@H]2Cc3nc(OC[C@@]45CCCN4C[C@@H](Cl)C5=O)nc(N4CC5CCC(C4)N5)c3CO2)c1C(F)(F)F ; CC#Cc1cc(N)c(F)c([C@@H]2Cc3nc(OC[C@@]45CCCN4C[C@@H](O)C5=O)nc(N4CC5CCC(C4)N5)c3CO2)c1C(F)(F)F]

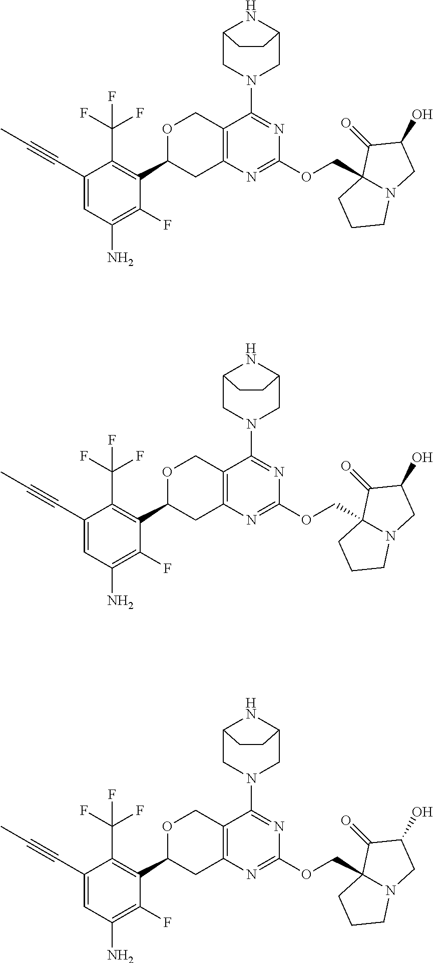 [SMILES: CC#Cc1cc(N)c(F)c([C@@H]2Cc3nc(OC[C@]45CCCN4C[C@H](O)C5=O)nc(N4CC5CCC(C4)N5)c3CO2)c1C(F)(F)F ; CC#Cc1cc(N)c(F)c([C@@H]2Cc3nc(OC[C@@]45CCCN4C[C@H](O)C5=O)nc(N4CC5CCC(C4)N5)c3CO2)c1C(F)(F)F ; CC#Cc1cc(N)c(F)c([C@@H]2Cc3nc(OC[C@]45CCCN4C[C@@H](O)C5=O)nc(N4CC5CCC(C4)N5)c3CO2)c1C(F)(F)F]

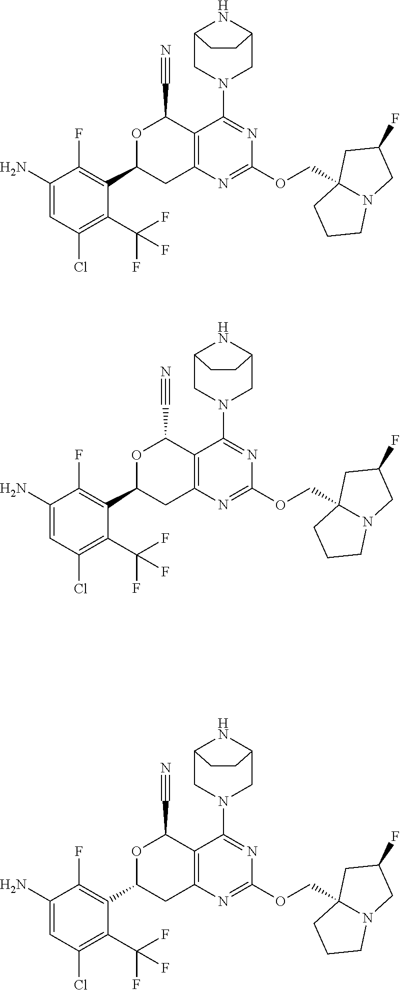 [SMILES: N#C[C@@H]1O[C@H](c2c(F)c(N)cc(Cl)c2C(F)(F)F)Cc2nc(OC[C@@]34CCCN3C[C@H](F)C4)nc(N3CC4CCC(C3)N4)c21 ; N#C[C@H]1O[C@H](c2c(F)c(N)cc(Cl)c2C(F)(F)F)Cc2nc(OC[C@@]34CCCN3C[C@H](F)C4)nc(N3CC4CCC(C3)N4)c21 ; N#C[C@@H]1O[C@@H](c2c(F)c(N)cc(Cl)c2C(F)(F)F)Cc2nc(OC[C@@]34CCCN3C[C@H](F)C4)nc(N3CC4CCC(C3)N4)c21]

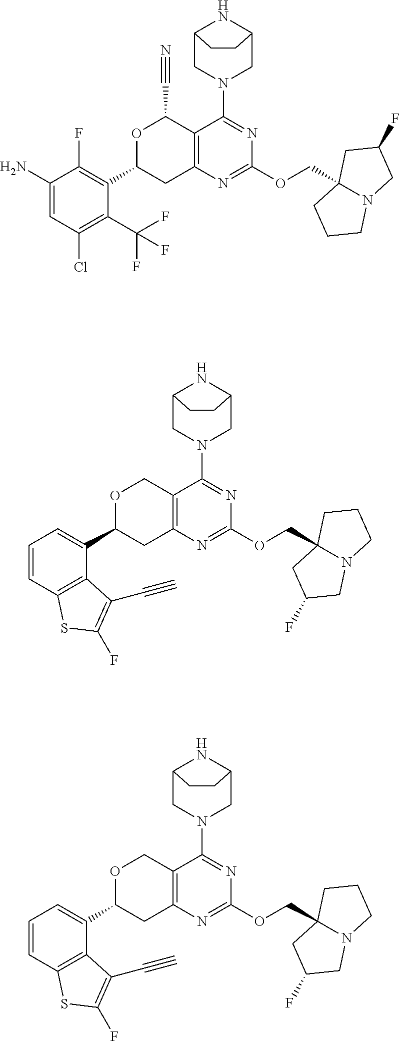 [SMILES: N#C[C@H]1O[C@@H](c2c(F)c(N)cc(Cl)c2C(F)(F)F)Cc2nc(OC[C@@]34CCCN3C[C@H](F)C4)nc(N3CC4CCC(C3)N4)c21 ; C#Cc1c(F)sc2cccc([C@@H]3Cc4nc(OC[C@@]56CCCN5C[C@H](F)C6)nc(N5CC6CCC(C5)N6)c4CO3)c12 ; C#Cc1c(F)sc2cccc([C@H]3Cc4nc(OC[C@@]56CCCN5C[C@H](F)C6)nc(N5CC6CCC(C5)N6)c4CO3)c12]

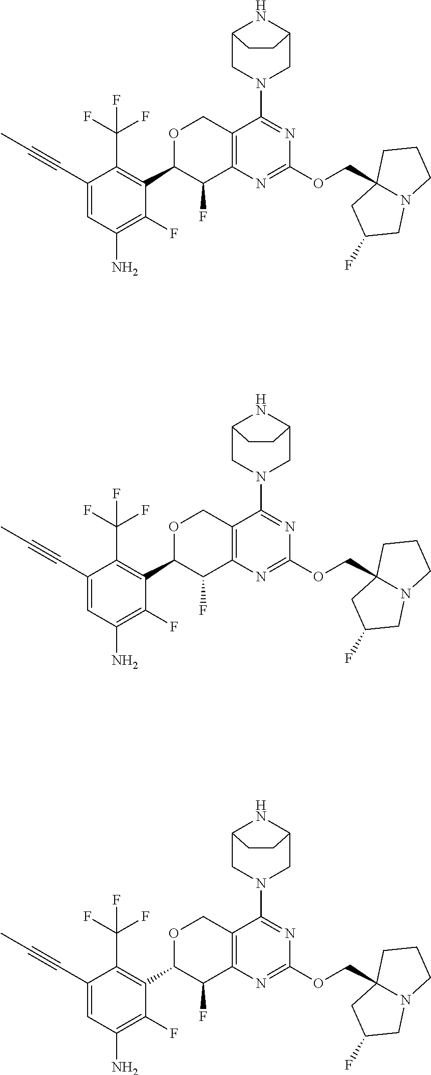 [SMILES: CC#Cc1cc(N)c(F)c([C@H]2OCc3c(nc(OC[C@@]45CCCN4C[C@H](F)C5)nc3N3CC4CCC(C3)N4)[C@H]2F)c1C(F)(F)F ; CC#Cc1cc(N)c(F)c([C@H]2OCc3c(nc(OC[C@@]45CCCN4C[C@H](F)C5)nc3N3CC4CCC(C3)N4)[C@@H]2F)c1C(F)(F)F ; CC#Cc1cc(N)c(F)c([C@@H]2OCc3c(nc(OC[C@@]45CCCN4C[C@H](F)C5)nc3N3CC4CCC(C3)N4)[C@H]2F)c1C(F)(F)F]

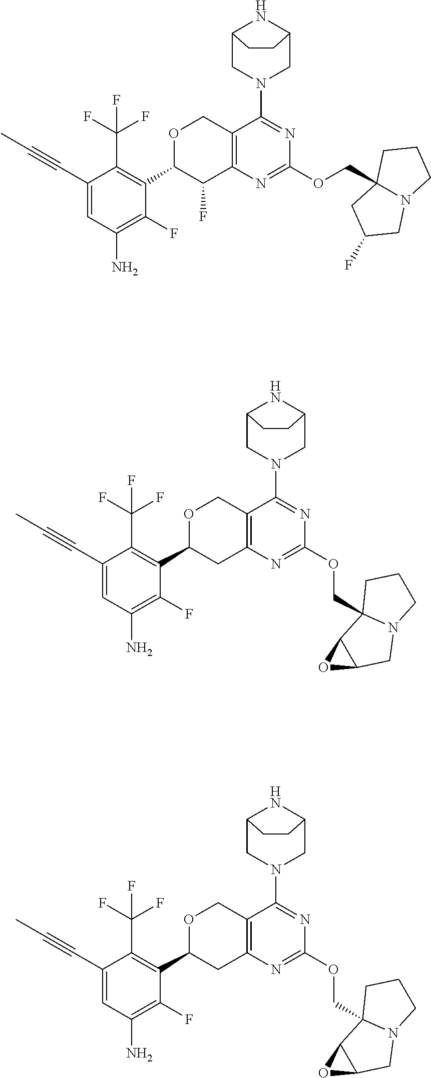 [SMILES: CC#Cc1cc(N)c(F)c([C@@H]2OCc3c(nc(OC[C@@]45CCCN4C[C@H](F)C5)nc3N3CC4CCC(C3)N4)[C@@H]2F)c1C(F)(F)F ; CC#Cc1cc(N)c(F)c([C@@H]2Cc3nc(OC[C@@]45CCCN4C[C@H]4O[C@H]45)nc(N4CC5CCC(C4)N5)c3CO2)c1C(F)(F)F ; CC#Cc1cc(N)c(F)c([C@@H]2Cc3nc(OC[C@]45CCCN4C[C@H]4O[C@H]45)nc(N4CC5CCC(C4)N5)c3CO2)c1C(F)(F)F]

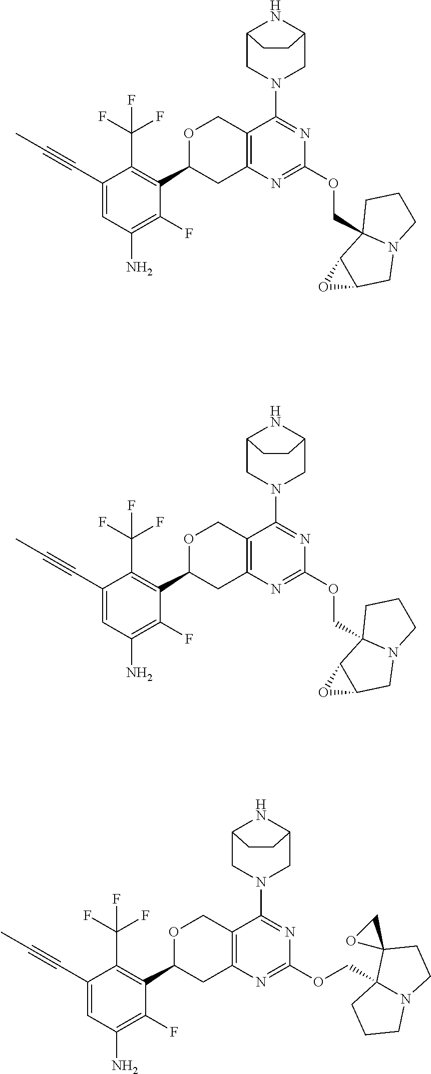 [SMILES: CC#Cc1cc(N)c(F)c([C@@H]2Cc3nc(OC[C@@]45CCCN4C[C@@H]4O[C@@H]45)nc(N4CC5CCC(C4)N5)c3CO2)c1C(F)(F)F ; CC#Cc1cc(N)c(F)c([C@@H]2Cc3nc(OC[C@]45CCCN4C[C@@H]4O[C@@H]45)nc(N4CC5CCC(C4)N5)c3CO2)c1C(F)(F)F ; CC#Cc1cc(N)c(F)c([C@@H]2Cc3nc(OC[C@@]45CCCN4CC[C@]54CO4)nc(N4CC5CCC(C4)N5)c3CO2)c1C(F)(F)F]

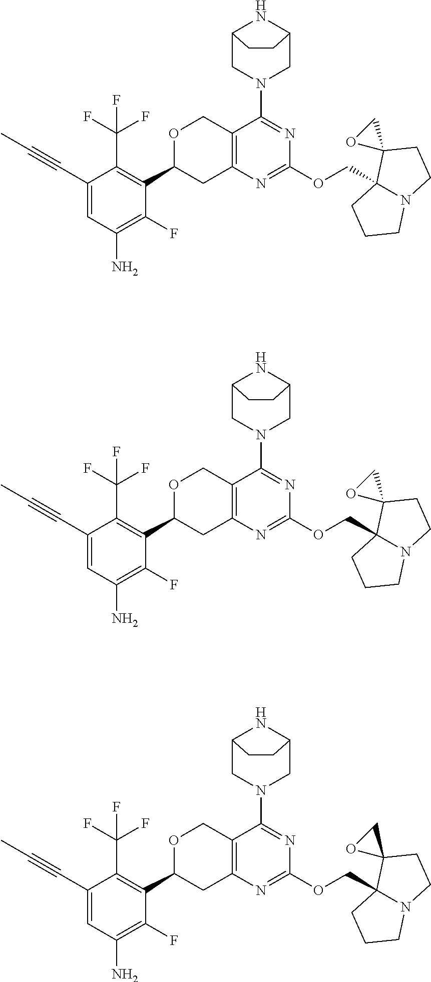 [SMILES: CC#Cc1cc(N)c(F)c([C@@H]2Cc3nc(OC[C@@]45CCCN4CC[C@@]54CO4)nc(N4CC5CCC(C4)N5)c3CO2)c1C(F)(F)F ; CC#Cc1cc(N)c(F)c([C@@H]2Cc3nc(OC[C@]45CCCN4CC[C@@]54CO4)nc(N4CC5CCC(C4)N5)c3CO2)c1C(F)(F)F ; CC#Cc1cc(N)c(F)c([C@@H]2Cc3nc(OC[C@]45CCCN4CC[C@]54CO4)nc(N4CC5CCC(C4)N5)c3CO2)c1C(F)(F)F]

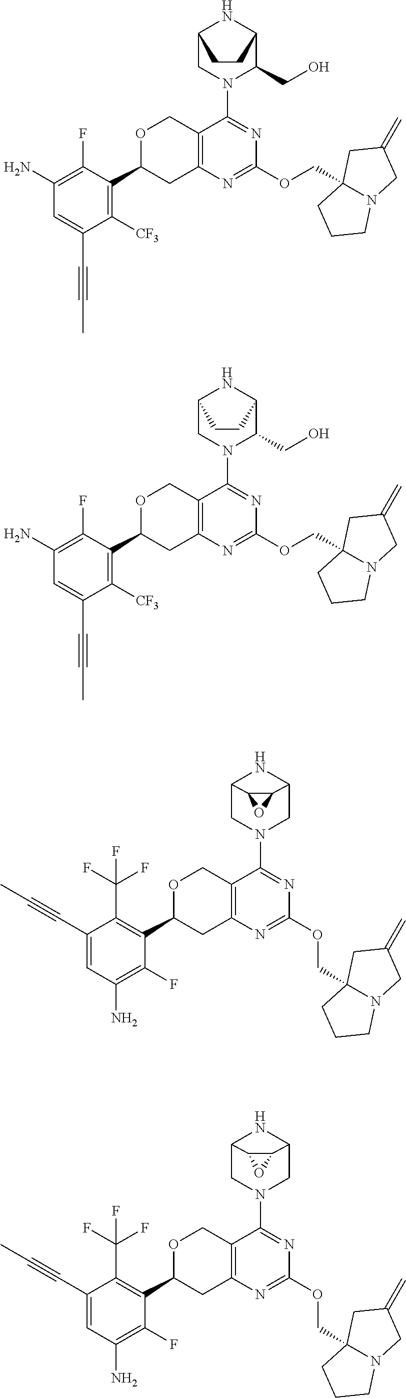

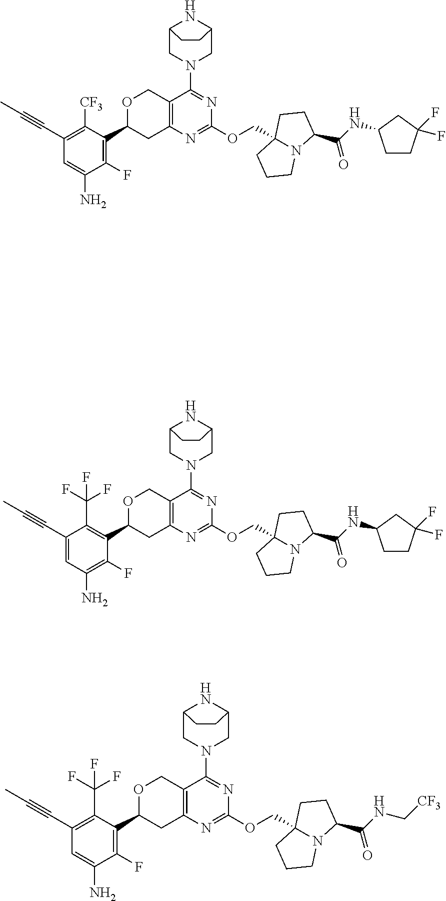 [SMILES: CC#Cc1cc(N)c(F)c([C@@H]2Cc3nc(OC[C@@]45CCCN4[C@H](C(=O)N[C@H]4CCC(F)(F)C4)CC5)nc(N4CC5CCC(C4)N5)c3CO2)c1C(F)(F)F ; CC#Cc1cc(N)c(F)c([C@@H]2Cc3nc(OC[C@@]45CCCN4[C@H](C(=O)N[C@@H]4CCC(F)(F)C4)CC5)nc(N4CC5CCC(C4)N5)c3CO2)c1C(F)(F)F ; CC#Cc1cc(N)c(F)c([C@@H]2Cc3nc(OC[C@@]45CCCN4[C@H](C(=O)NCC(F)(F)F)CC5)nc(N4CC5CCC(C4)N5)c3CO2)c1C(F)(F)F]

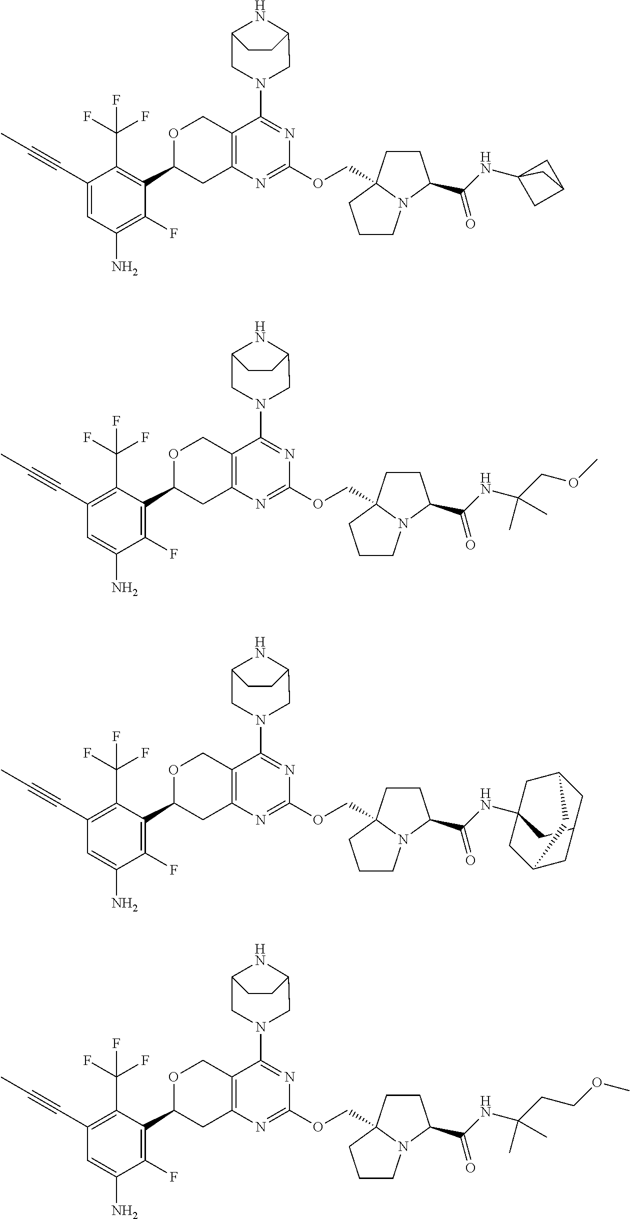

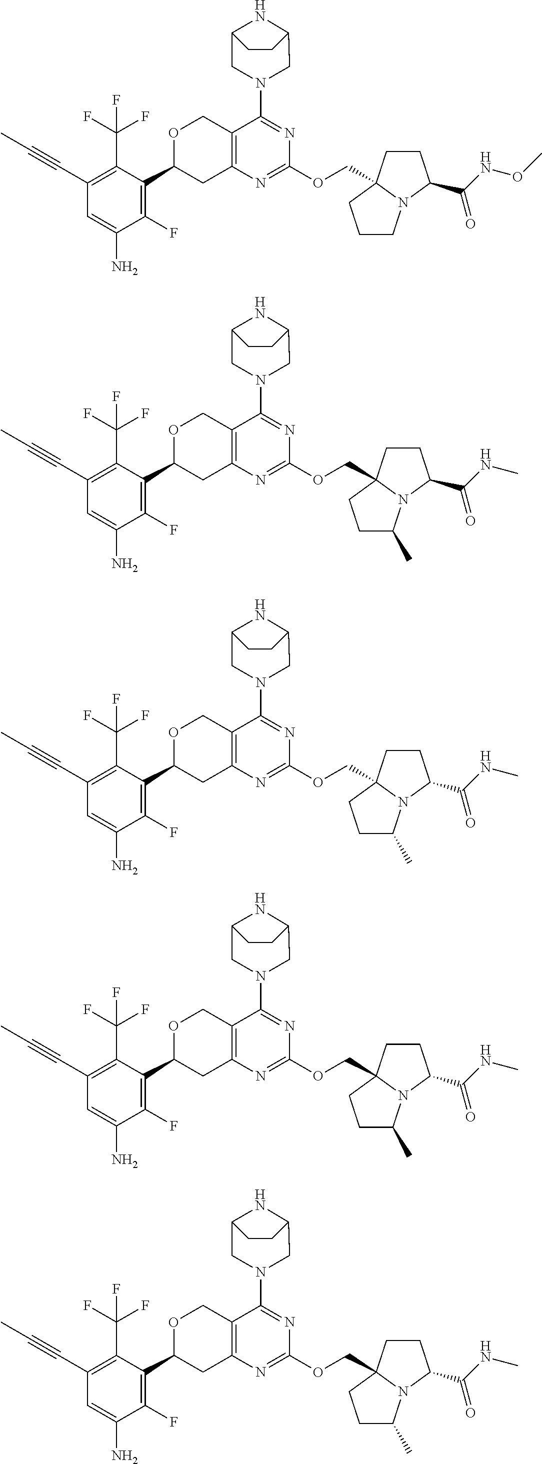

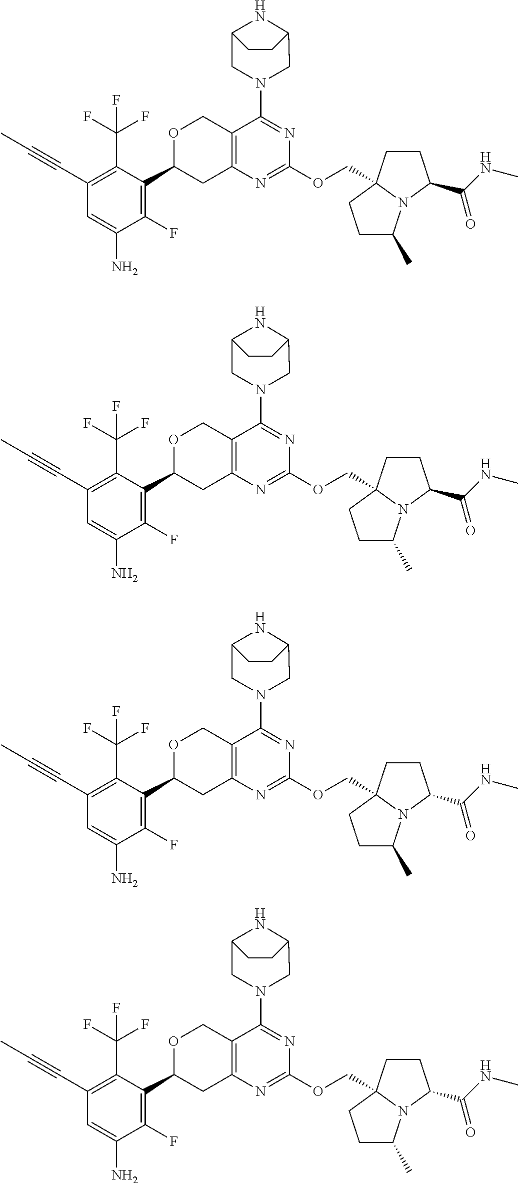

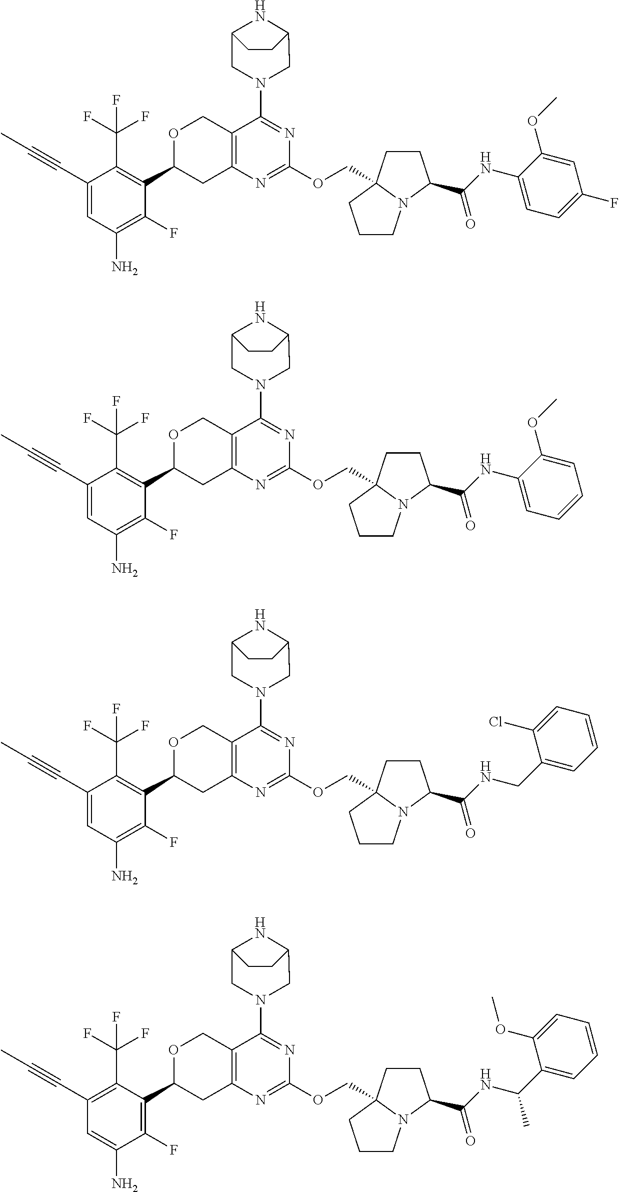 [SMILES: CC#Cc1cc(N)c(F)c([C@@H]2Cc3nc(OC[C@@]45CCCN4[C@H](C(=O)Nc4ccc(F)cc4OC)CC5)nc(N4CC5CCC(C4)N5)c3CO2)c1C(F)(F)F ; CC#Cc1cc(N)c(F)c([C@@H]2Cc3nc(OC[C@@]45CCCN4[C@H](C(=O)Nc4ccccc4OC)CC5)nc(N4CC5CCC(C4)N5)c3CO2)c1C(F)(F)F ; CC#Cc1cc(N)c(F)c([C@@H]2Cc3nc(OC[C@@]45CCCN4[C@H](C(=O)NCc4ccccc4Cl)CC5)nc(N4CC5CCC(C4)N5)c3CO2)c1C(F)(F)F ; CC#Cc1cc(N)c(F)c([C@@H]2Cc3nc(OC[C@@]45CCCN4[C@H](C(=O)N[C@@H](C)c4ccccc4OC)CC5)nc(N4CC5CCC(C4)N5)c3CO2)c1C(F)(F)F]

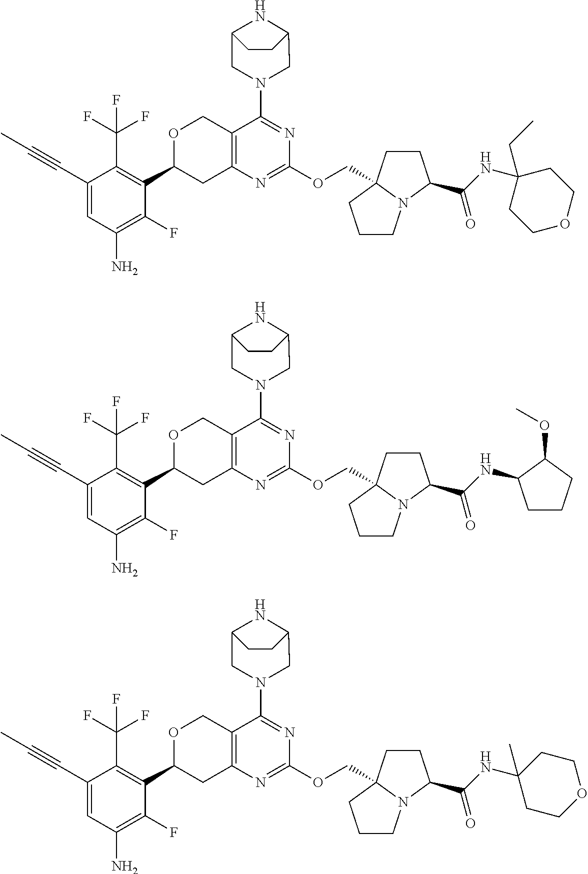 [SMILES: CC#Cc1cc(N)c(F)c([C@@H]2Cc3nc(OC[C@@]45CCCN4[C@H](C(=O)NC4(CC)CCOCC4)CC5)nc(N4CC5CCC(C4)N5)c3CO2)c1C(F)(F)F ; CC#Cc1cc(N)c(F)c([C@@H]2Cc3nc(OC[C@@]45CCCN4[C@H](C(=O)N[C@@H]4CCC[C@@H]4OC)CC5)nc(N4CC5CCC(C4)N5)c3CO2)c1C(F)(F)F ; CC#Cc1cc(N)c(F)c([C@@H]2Cc3nc(OC[C@@]45CCCN4[C@H](C(=O)NC4(C)CCOCC4)CC5)nc(N4CC5CCC(C4)N5)c3CO2)c1C(F)(F)F]

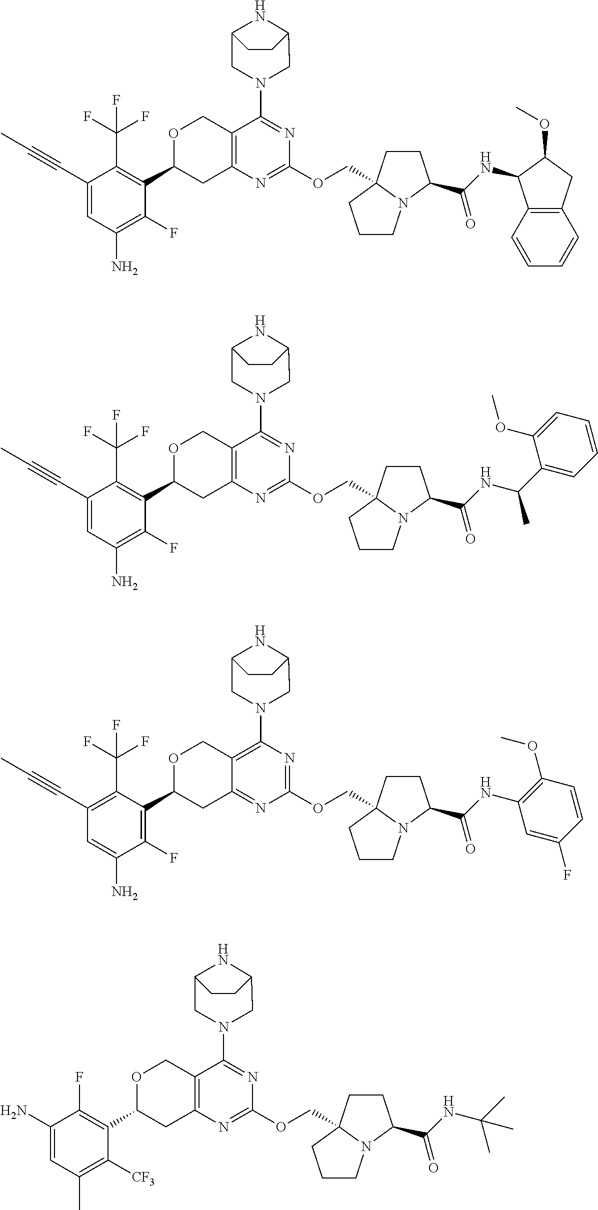 [SMILES: CC#Cc1cc(N)c(F)c([C@@H]2Cc3nc(OC[C@@]45CCCN4[C@H](C(=O)N[C@@H]4c6ccccc6C[C@@H]4OC)CC5)nc(N4CC5CCC(C4)N5)c3CO2)c1C(F)(F)F ; CC#Cc1cc(N)c(F)c([C@@H]2Cc3nc(OC[C@@]45CCCN4[C@H](C(=O)N[C@H](C)c4ccccc4OC)CC5)nc(N4CC5CCC(C4)N5)c3CO2)c1C(F)(F)F ; CC#Cc1cc(N)c(F)c([C@@H]2Cc3nc(OC[C@@]45CCCN4[C@H](C(=O)Nc4cc(F)ccc4OC)CC5)nc(N4CC5CCC(C4)N5)c3CO2)c1C(F)(F)F ; Cc1cc(N)c(F)c([C@H]2Cc3nc(OC[C@@]45CCCN4[C@H](C(=O)NC(C)(C)C)CC5)nc(N4CC5CCC(C4)N5)c3CO2)c1C(F)(F)F]

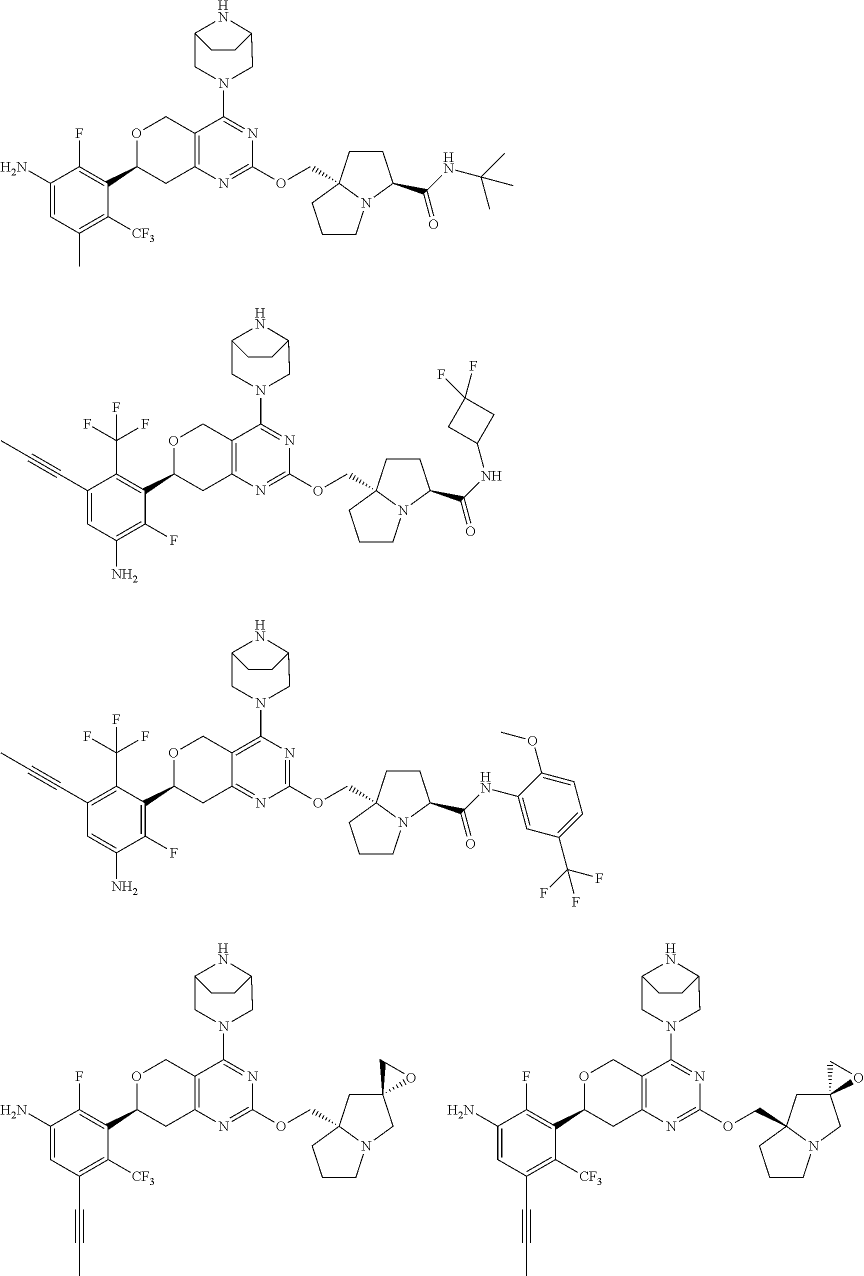

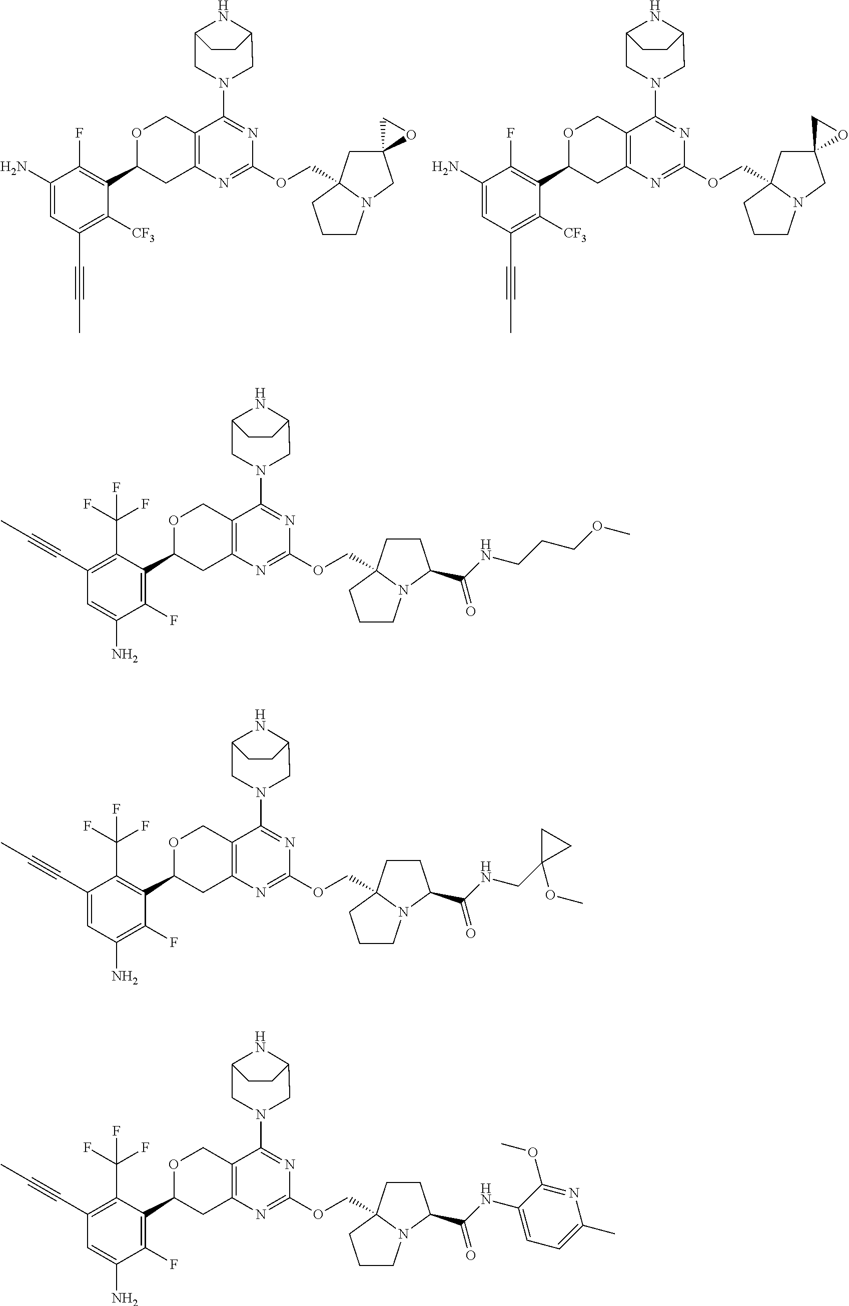

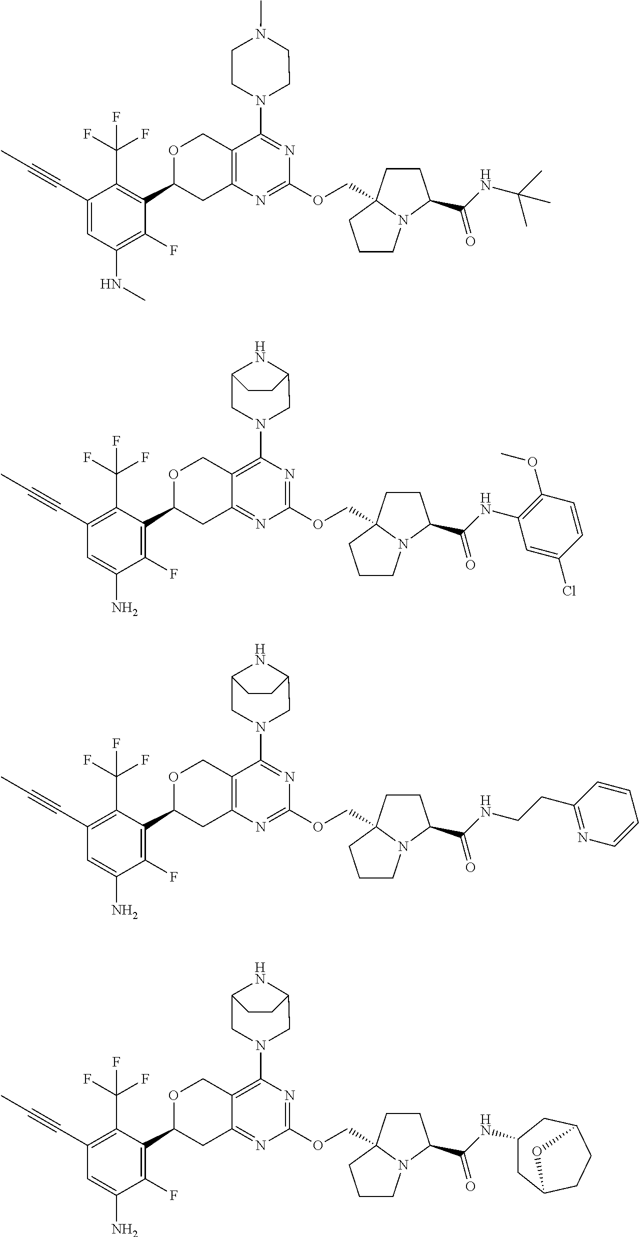

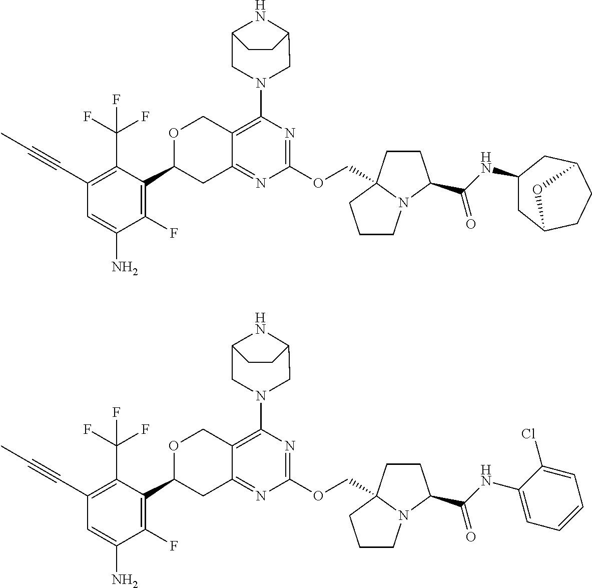 [SMILES: CC#Cc1cc(N)c(F)c([C@@H]2Cc3nc(OC[C@@]45CCCN4[C@H](C(=O)N[C@H]4C[C@H]6CC[C@@H](C4)O6)CC5)nc(N4CC5CCC(C4)N5)c3CO2)c1C(F)(F)F ; CC#Cc1cc(N)c(F)c([C@@H]2Cc3nc(OC[C@@]45CCCN4[C@H](C(=O)Nc4ccccc4Cl)CC5)nc(N4CC5CCC(C4)N5)c3CO2)c1C(F)(F)F]

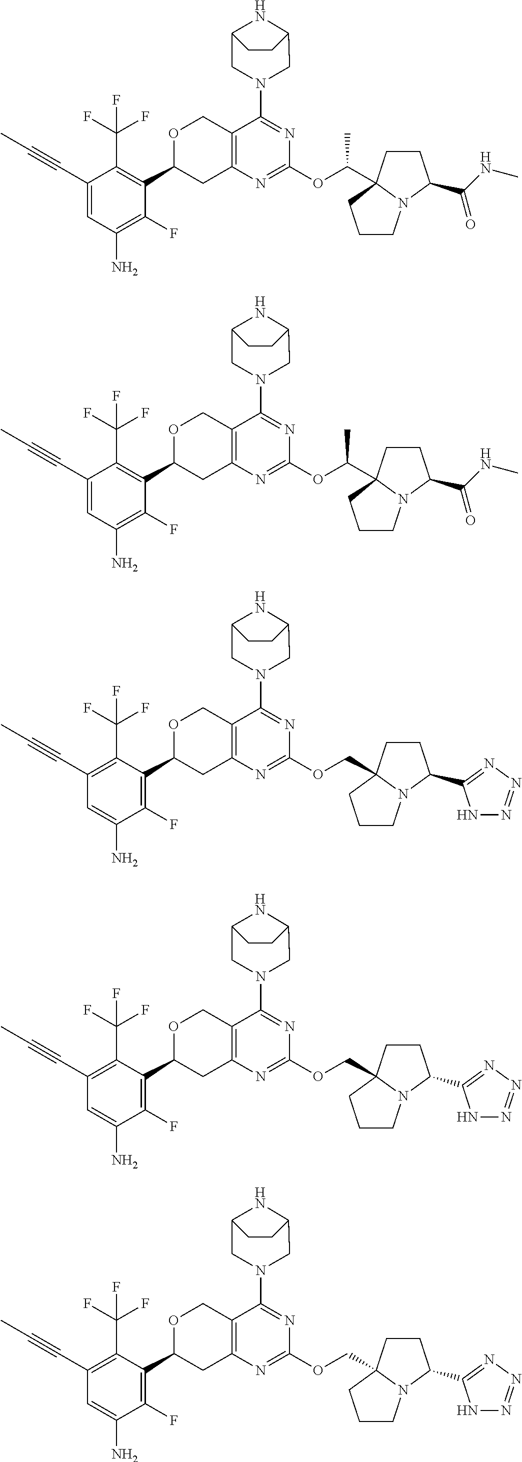

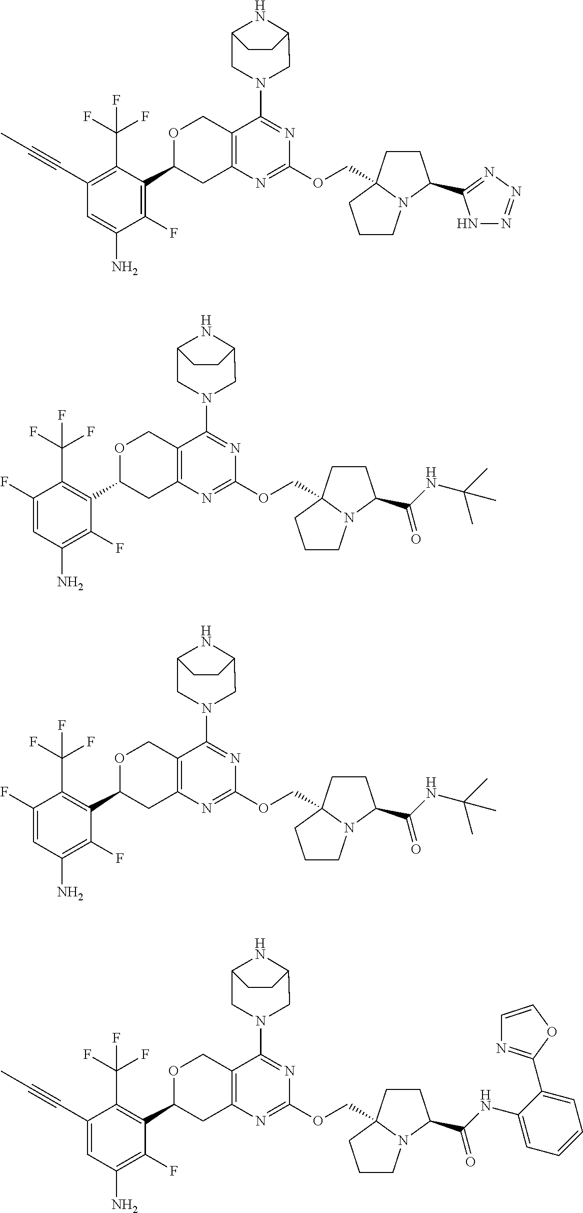

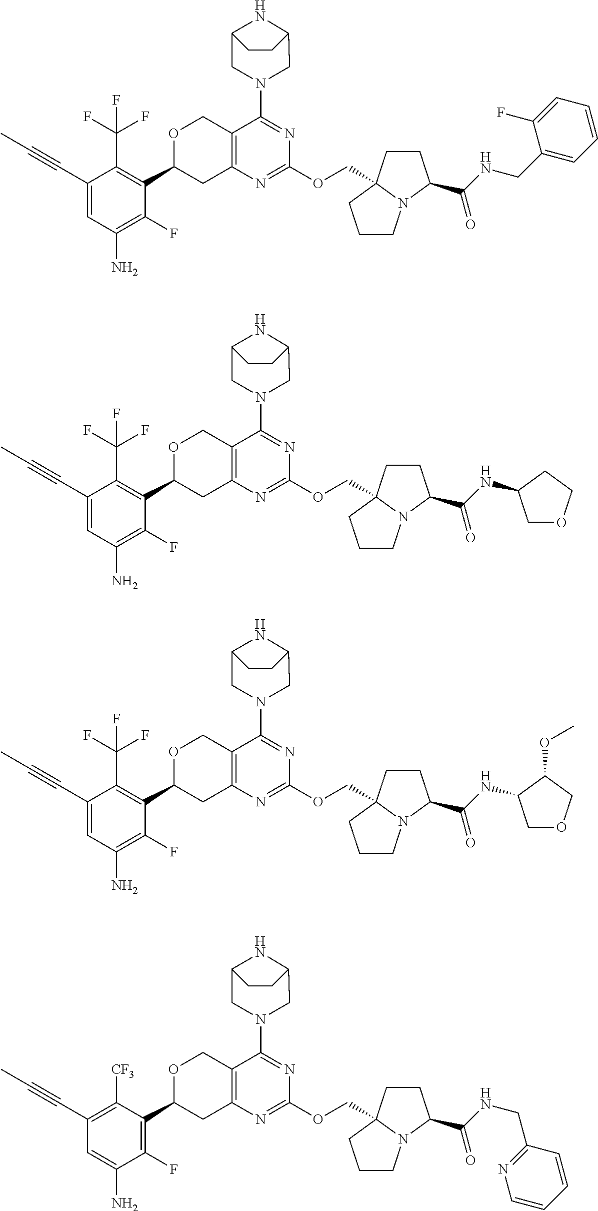

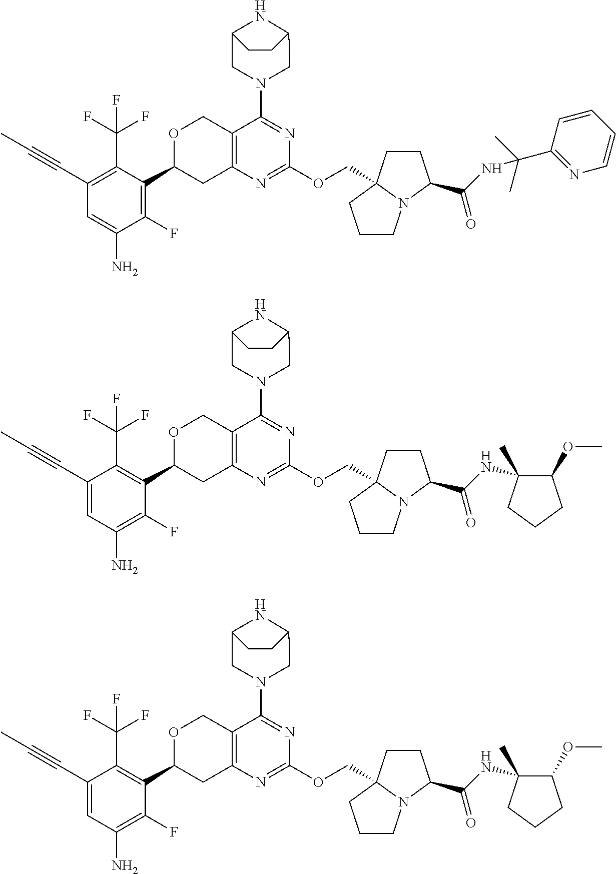

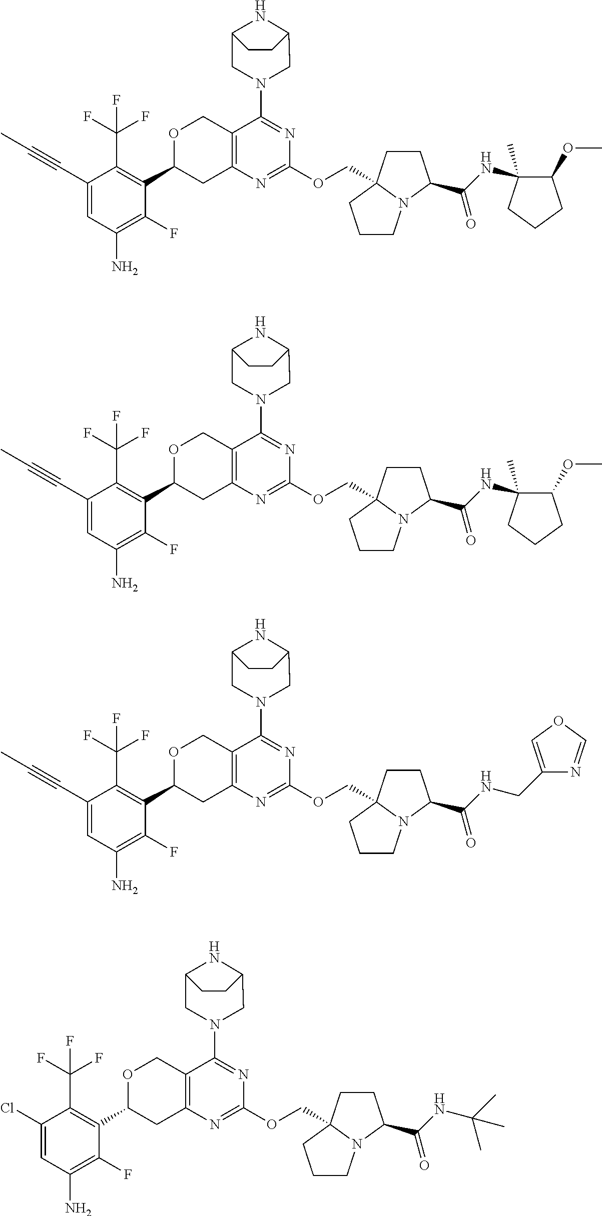

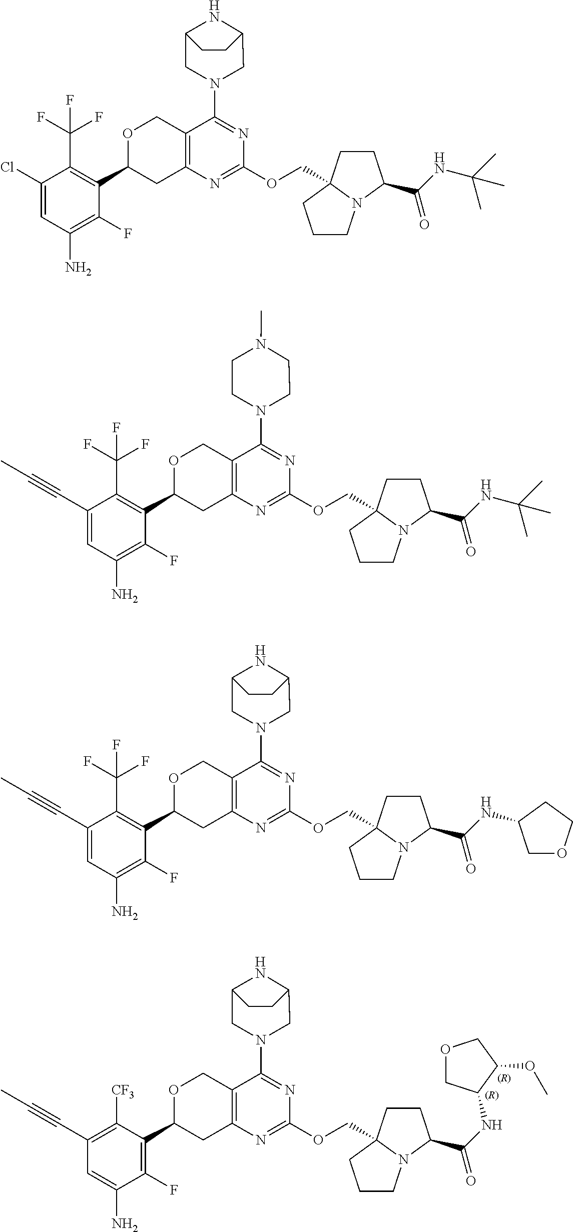

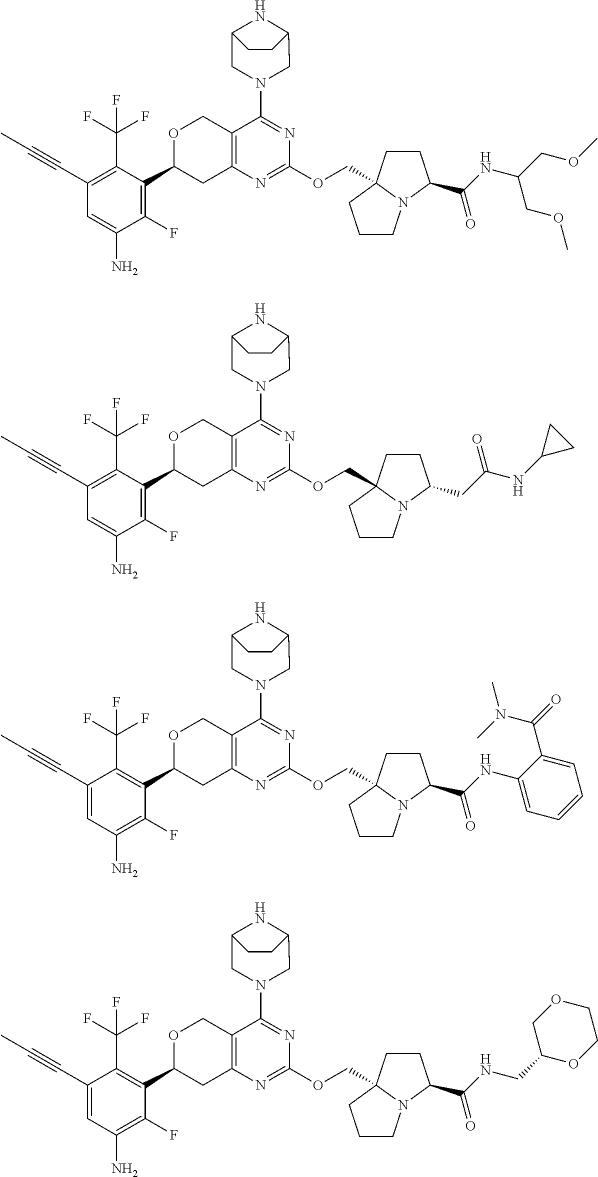

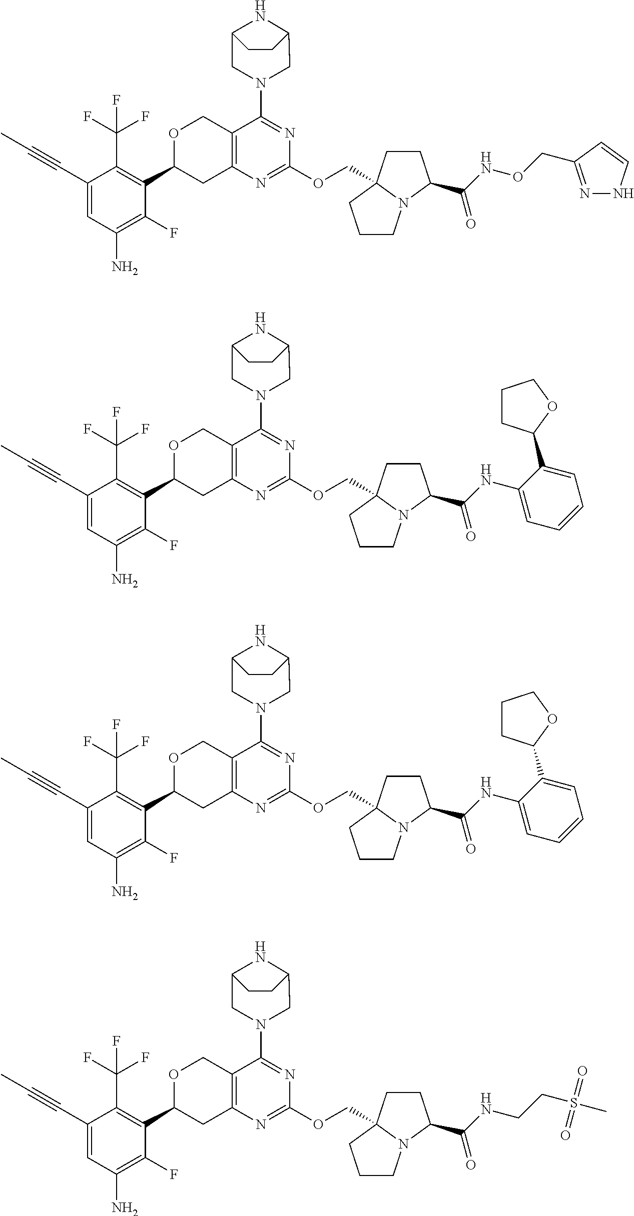 [SMILES: CC#Cc1cc(N)c(F)c([C@@H]2Cc3nc(OC[C@@]45CCCN4[C@H](C(=O)NOCc4cc[nH]n4)CC5)nc(N4CC5CCC(C4)N5)c3CO2)c1C(F)(F)F ; CC#Cc1cc(N)c(F)c([C@@H]2Cc3nc(OC[C@@]45CCCN4[C@H](C(=O)Nc4ccccc4[C@H]4CCCO4)CC5)nc(N4CC5CCC(C4)N5)c3CO2)c1C(F)(F)F ; CC#Cc1cc(N)c(F)c([C@@H]2Cc3nc(OC[C@@]45CCCN4[C@H](C(=O)Nc4ccccc4[C@@H]4CCCO4)CC5)nc(N4CC5CCC(C4)N5)c3CO2)c1C(F)(F)F ; CC#Cc1cc(N)c(F)c([C@@H]2Cc3nc(OC[C@@]45CCCN4[C@H](C(=O)NCCS(C)(=O)=O)CC5)nc(N4CC5CCC(C4)N5)c3CO2)c1C(F)(F)F]

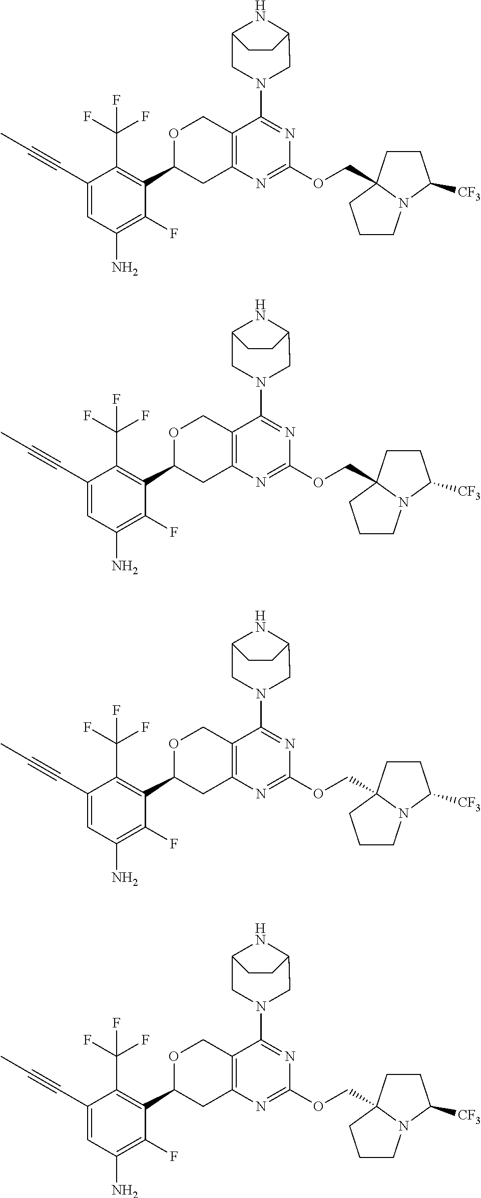 [SMILES: CC#Cc1cc(N)c(F)c([C@@H]2Cc3nc(OC[C@]45CCCN4[C@H](C(F)(F)F)CC5)nc(N4CC5CCC(C4)N5)c3CO2)c1C(F)(F)F ; CC#Cc1cc(N)c(F)c([C@@H]2Cc3nc(OC[C@]45CCCN4[C@@H](C(F)(F)F)CC5)nc(N4CC5CCC(C4)N5)c3CO2)c1C(F)(F)F ; CC#Cc1cc(N)c(F)c([C@@H]2Cc3nc(OC[C@@]45CCCN4[C@@H](C(F)(F)F)CC5)nc(N4CC5CCC(C4)N5)c3CO2)c1C(F)(F)F ; CC#Cc1cc(N)c(F)c([C@@H]2Cc3nc(OC[C@@]45CCCN4[C@H](C(F)(F)F)CC5)nc(N4CC5CCC(C4)N5)c3CO2)c1C(F)(F)F]

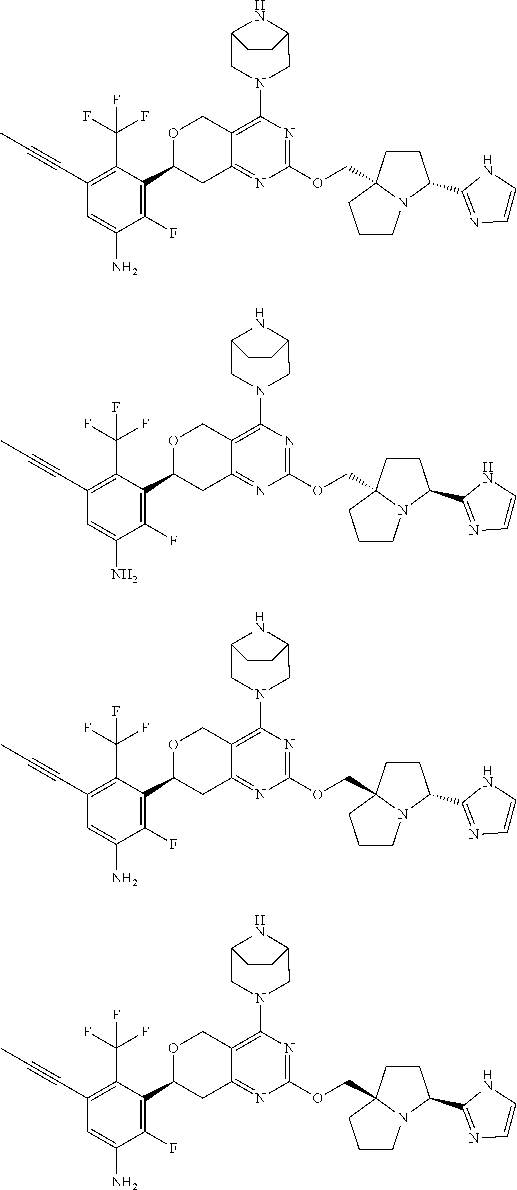 [SMILES: CC#Cc1cc(N)c(F)c([C@@H]2Cc3nc(OC[C@@]45CCCN4[C@@H](c4ncc[nH]4)CC5)nc(N4CC5CCC(C4)N5)c3CO2)c1C(F)(F)F ; CC#Cc1cc(N)c(F)c([C@@H]2Cc3nc(OC[C@@]45CCCN4[C@H](c4ncc[nH]4)CC5)nc(N4CC5CCC(C4)N5)c3CO2)c1C(F)(F)F ; CC#Cc1cc(N)c(F)c([C@@H]2Cc3nc(OC[C@]45CCCN4[C@@H](c4ncc[nH]4)CC5)nc(N4CC5CCC(C4)N5)c3CO2)c1C(F)(F)F ; CC#Cc1cc(N)c(F)c([C@@H]2Cc3nc(OC[C@]45CCCN4[C@H](c4ncc[nH]4)CC5)nc(N4CC5CCC(C4)N5)c3CO2)c1C(F)(F)F]

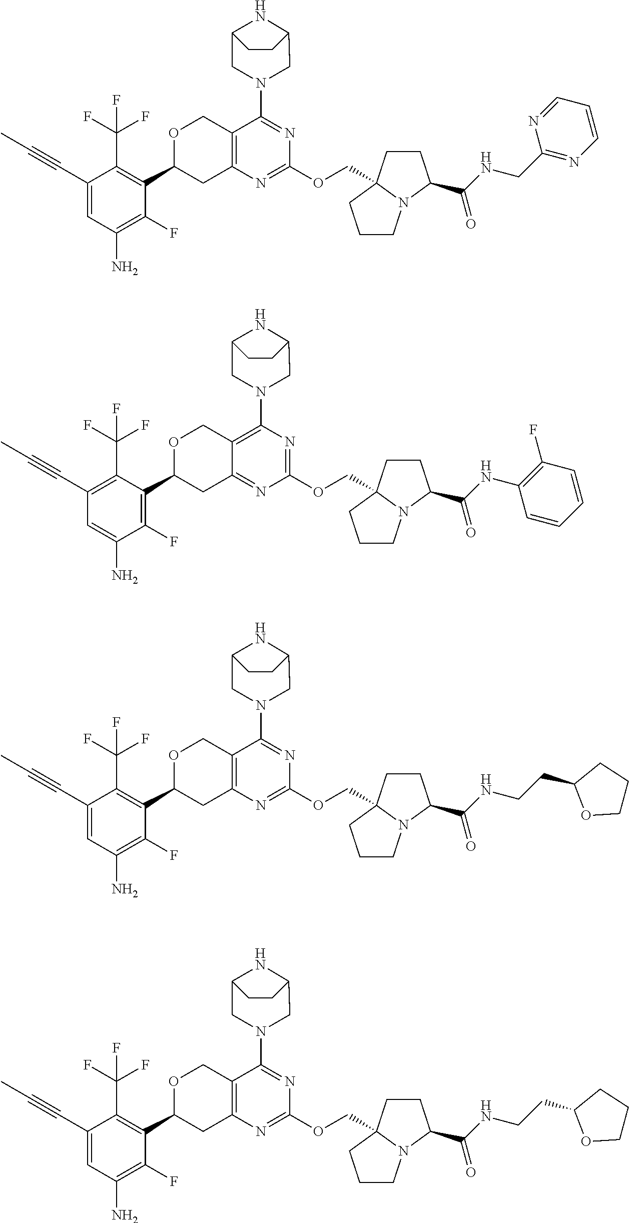 [SMILES: CC#Cc1cc(N)c(F)c([C@@H]2Cc3nc(OC[C@@]45CCCN4[C@H](C(=O)NCc4ncccn4)CC5)nc(N4CC5CCC(C4)N5)c3CO2)c1C(F)(F)F ; CC#Cc1cc(N)c(F)c([C@@H]2Cc3nc(OC[C@@]45CCCN4[C@H](C(=O)Nc4ccccc4F)CC5)nc(N4CC5CCC(C4)N5)c3CO2)c1C(F)(F)F ; CC#Cc1cc(N)c(F)c([C@@H]2Cc3nc(OC[C@@]45CCCN4[C@H](C(=O)NCC[C@H]4CCCO4)CC5)nc(N4CC5CCC(C4)N5)c3CO2)c1C(F)(F)F ; CC#Cc1cc(N)c(F)c([C@@H]2Cc3nc(OC[C@@]45CCCN4[C@H](C(=O)NCC[C@@H]4CCCO4)CC5)nc(N4CC5CCC(C4)N5)c3CO2)c1C(F)(F)F]

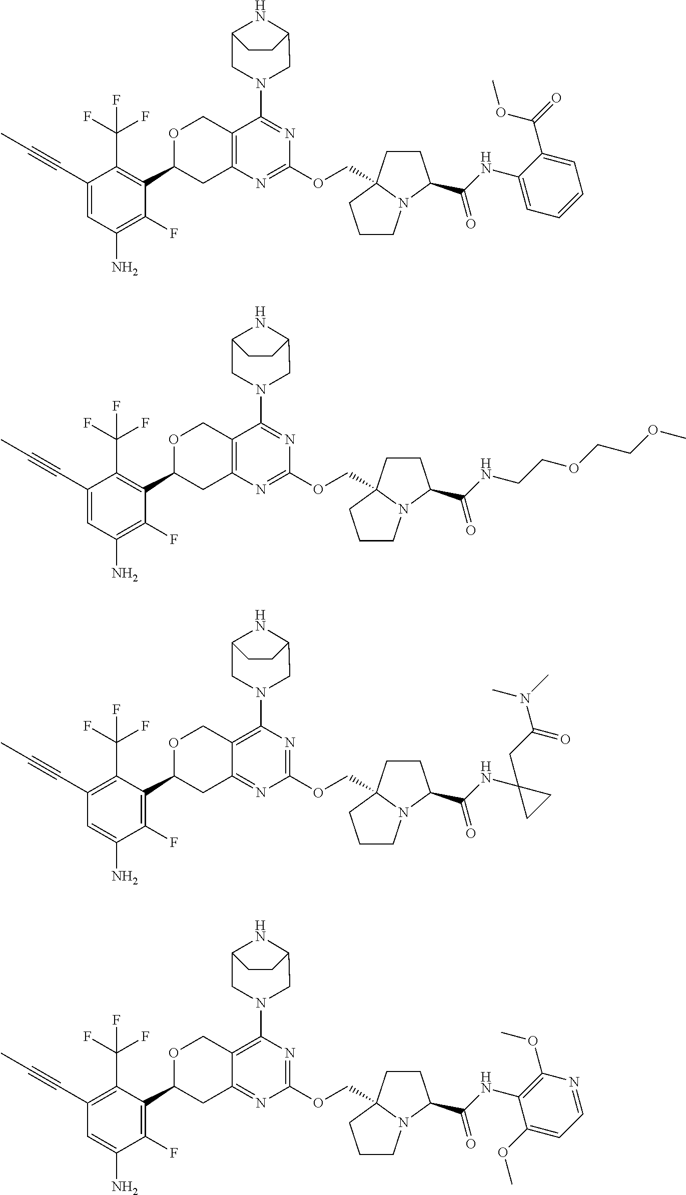

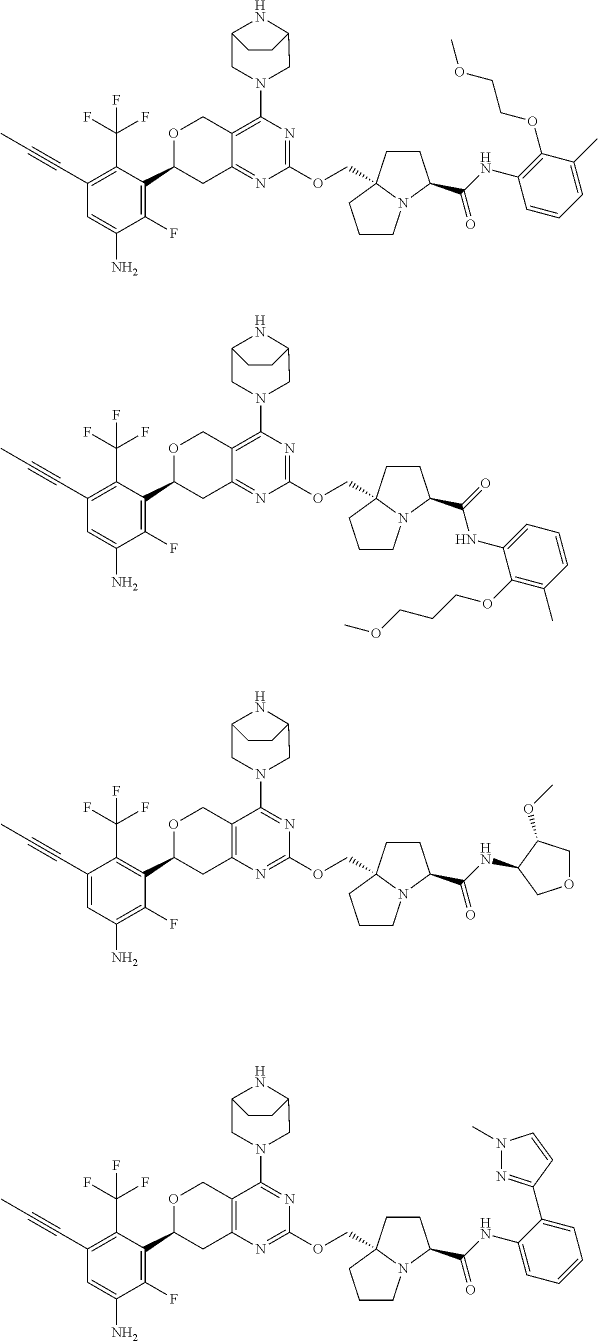

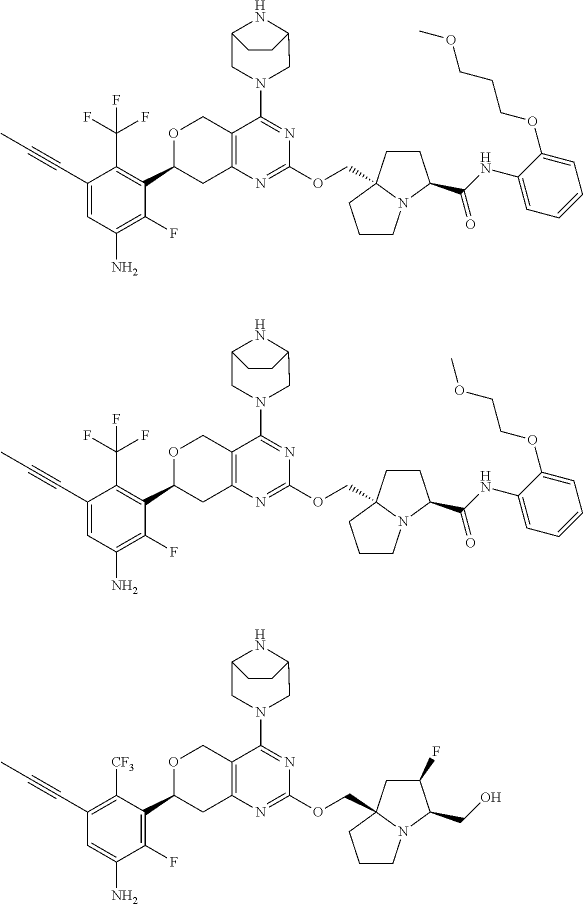 [SMILES: CC#Cc1cc(N)c(F)c([C@@H]2Cc3nc(OC[C@@]45CCCN4[C@H](C(=O)Nc4ccccc4OCCCOC)CC5)nc(N4CC5CCC(C4)N5)c3CO2)c1C(F)(F)F ; CC#Cc1cc(N)c(F)c([C@@H]2Cc3nc(OC[C@@]45CCCN4[C@H](C(=O)Nc4ccccc4OCCOC)CC5)nc(N4CC5CCC(C4)N5)c3CO2)c1C(F)(F)F ; CC#Cc1cc(N)c(F)c([C@@H]2Cc3nc(OC[C@]45CCCN4[C@H](CO)[C@H](F)C5)nc(N4CC5CCC(C4)N5)c3CO2)c1C(F)(F)F]

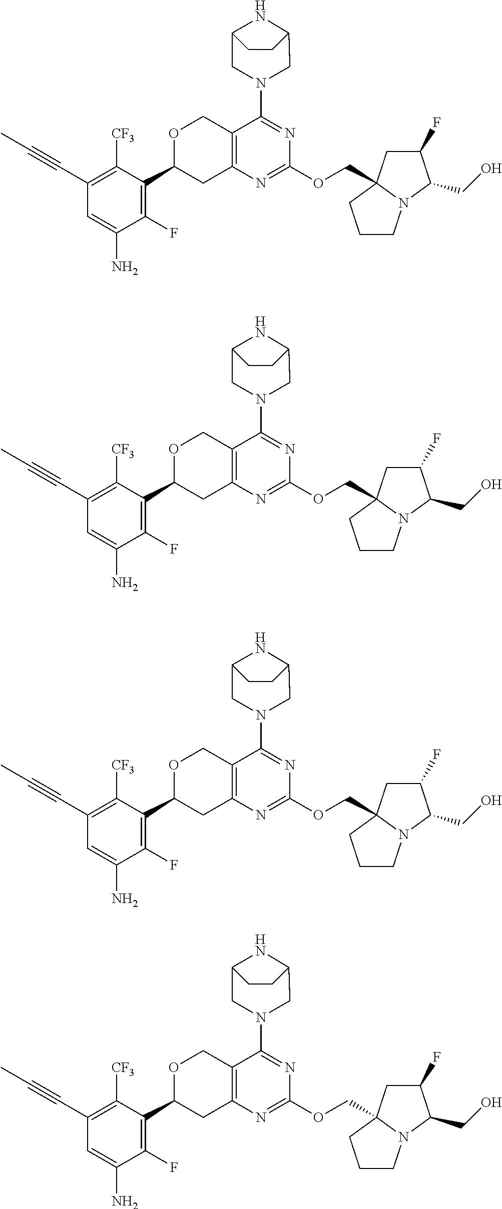 [SMILES: CC#Cc1cc(N)c(F)c([C@@H]2Cc3nc(OC[C@]45CCCN4[C@@H](CO)[C@H](F)C5)nc(N4CC5CCC(C4)N5)c3CO2)c1C(F)(F)F ; CC#Cc1cc(N)c(F)c([C@@H]2Cc3nc(OC[C@]45CCCN4[C@H](CO)[C@@H](F)C5)nc(N4CC5CCC(C4)N5)c3CO2)c1C(F)(F)F ; CC#Cc1cc(N)c(F)c([C@@H]2Cc3nc(OC[C@]45CCCN4[C@@H](CO)[C@@H](F)C5)nc(N4CC5CCC(C4)N5)c3CO2)c1C(F)(F)F ; CC#Cc1cc(N)c(F)c([C@@H]2Cc3nc(OC[C@@]45CCCN4[C@H](CO)[C@H](F)C5)nc(N4CC5CCC(C4)N5)c3CO2)c1C(F)(F)F]

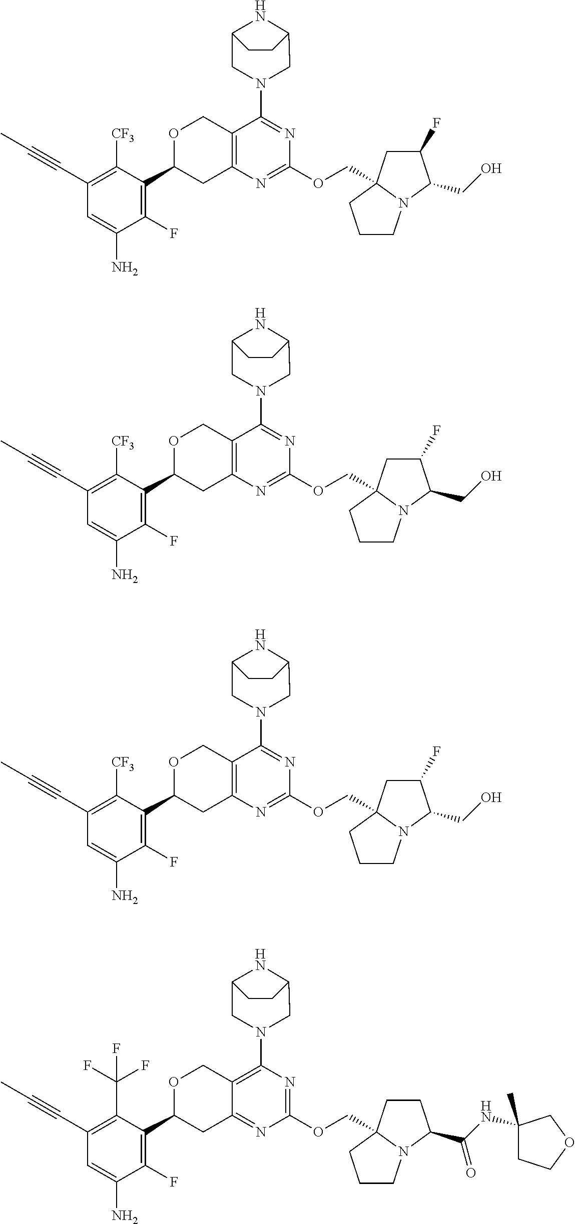 [SMILES: CC#Cc1cc(N)c(F)c([C@@H]2Cc3nc(OC[C@@]45CCCN4[C@@H](CO)[C@H](F)C5)nc(N4CC5CCC(C4)N5)c3CO2)c1C(F)(F)F ; CC#Cc1cc(N)c(F)c([C@@H]2Cc3nc(OC[C@@]45CCCN4[C@H](CO)[C@@H](F)C5)nc(N4CC5CCC(C4)N5)c3CO2)c1C(F)(F)F ; CC#Cc1cc(N)c(F)c([C@@H]2Cc3nc(OC[C@@]45CCCN4[C@@H](CO)[C@@H](F)C5)nc(N4CC5CCC(C4)N5)c3CO2)c1C(F)(F)F ; CC#Cc1cc(N)c(F)c([C@@H]2Cc3nc(OC[C@@]45CCCN4[C@H](C(=O)N[C@@]4(C)CCOC4)CC5)nc(N4CC5CCC(C4)N5)c3CO2)c1C(F)(F)F]

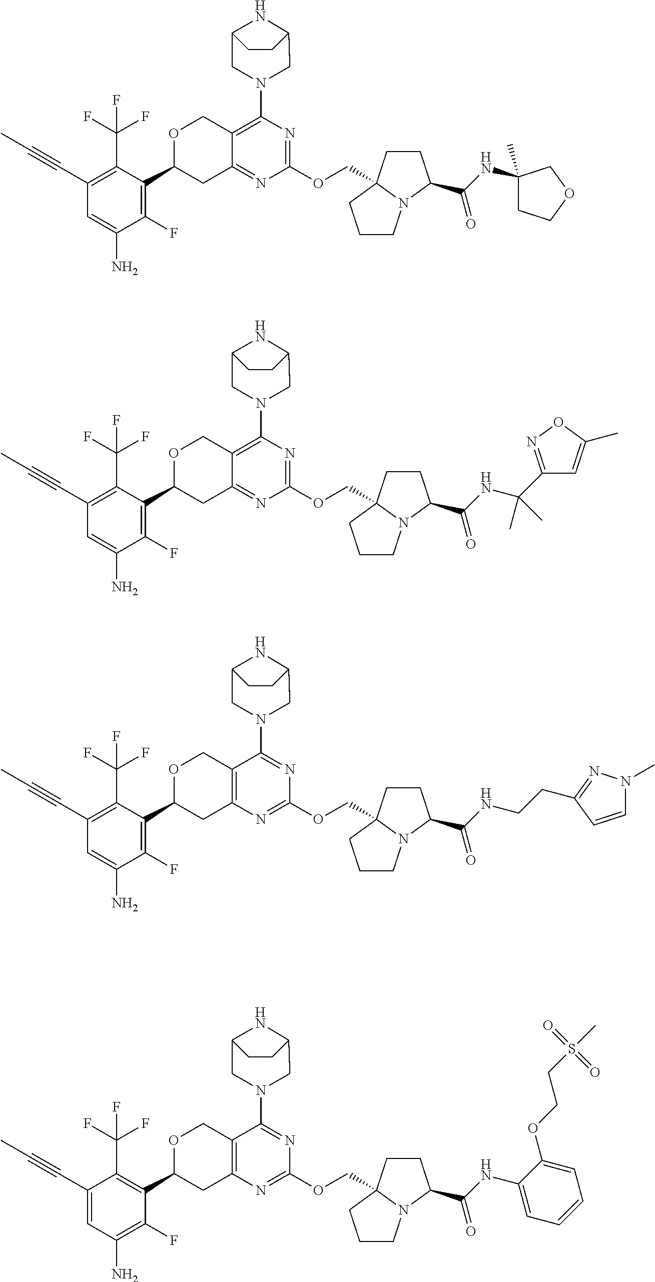

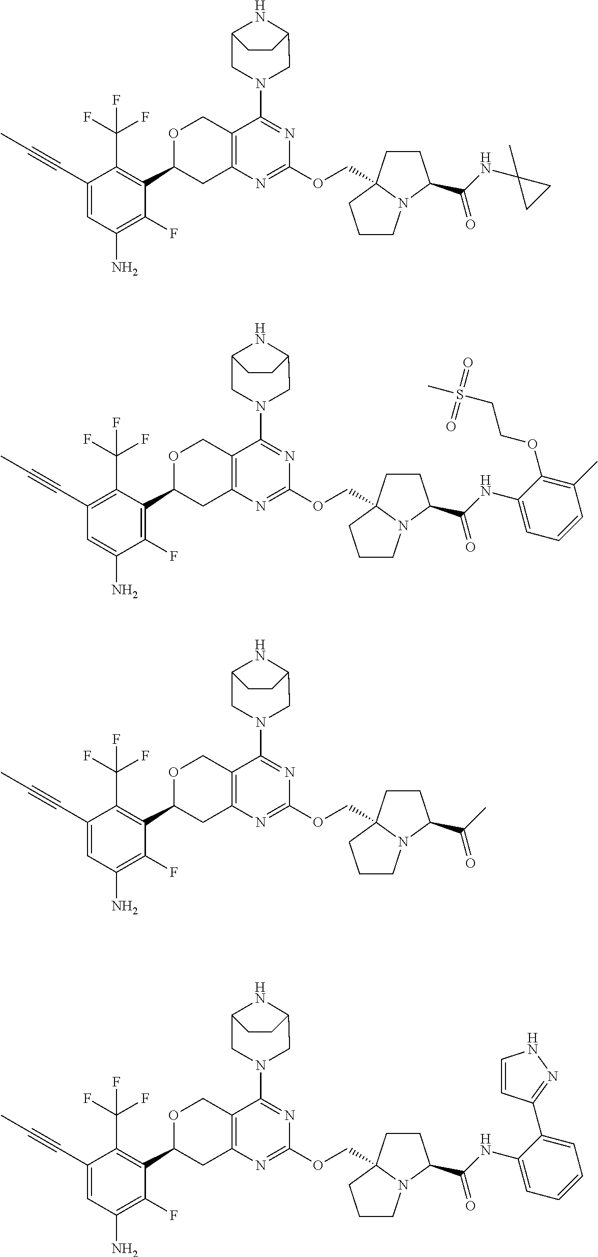

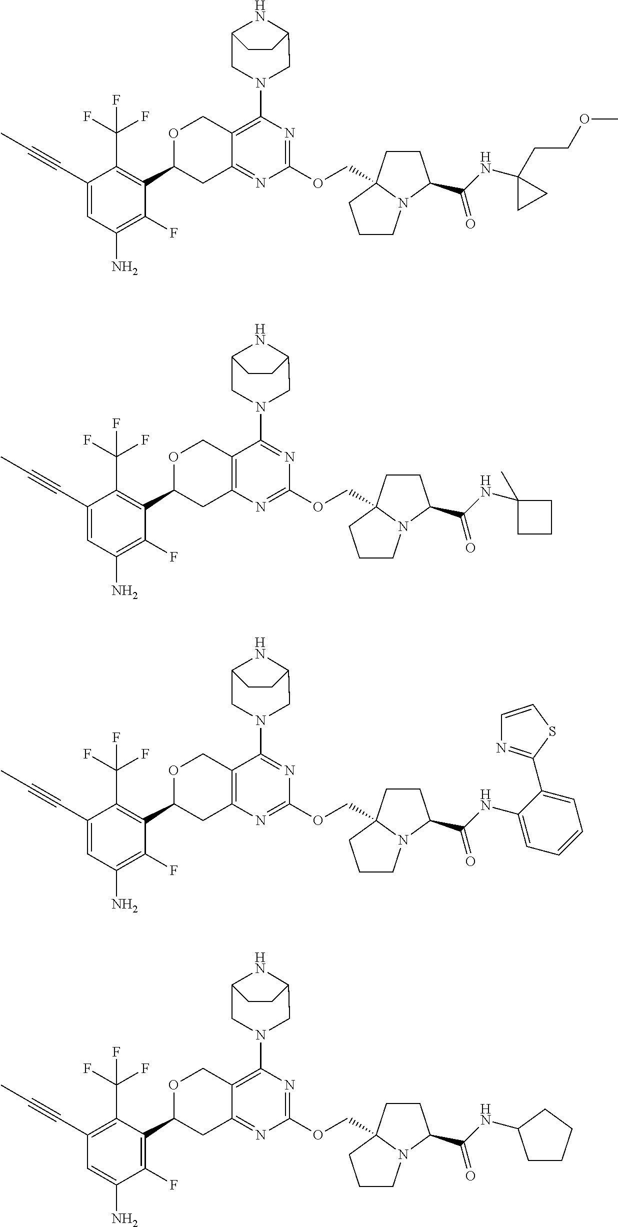

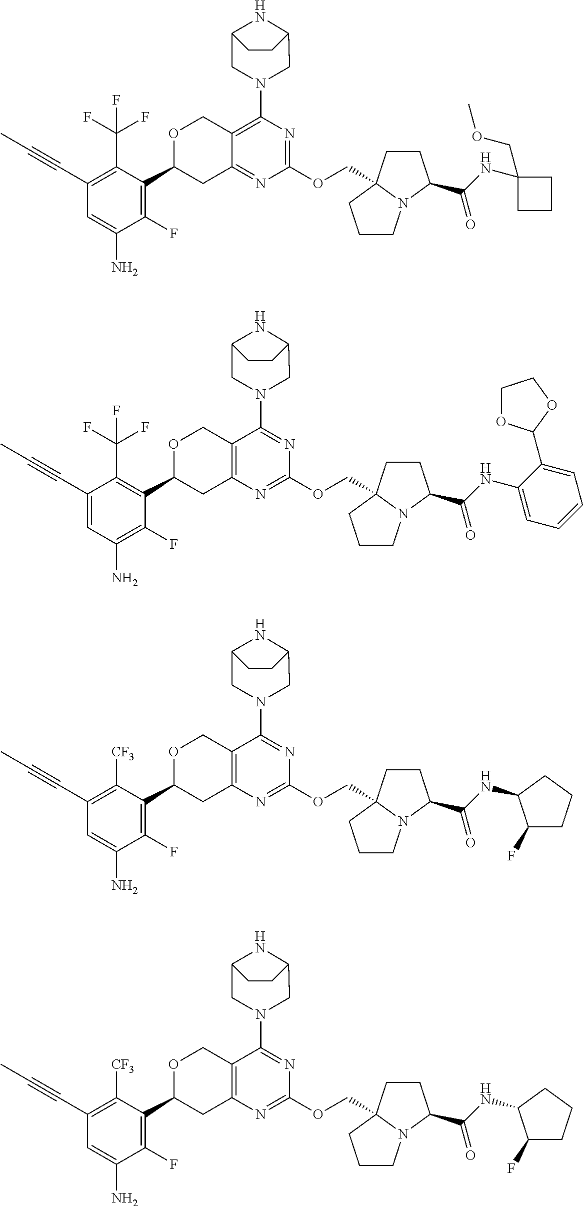 [SMILES: CC#Cc1cc(N)c(F)c([C@@H]2Cc3nc(OC[C@@]45CCCN4[C@H](C(=O)NC4(COC)CCC4)CC5)nc(N4CC5CCC(C4)N5)c3CO2)c1C(F)(F)F ; CC#Cc1cc(N)c(F)c([C@@H]2Cc3nc(OC[C@@]45CCCN4[C@H](C(=O)Nc4ccccc4C4OCCO4)CC5)nc(N4CC5CCC(C4)N5)c3CO2)c1C(F)(F)F ; CC#Cc1cc(N)c(F)c([C@@H]2Cc3nc(OC[C@@]45CCCN4[C@H](C(=O)N[C@H]4CCC[C@H]4F)CC5)nc(N4CC5CCC(C4)N5)c3CO2)c1C(F)(F)F ; CC#Cc1cc(N)c(F)c([C@@H]2Cc3nc(OC[C@@]45CCCN4[C@H](C(=O)N[C@@H]4CCC[C@H]4F)CC5)nc(N4CC5CCC(C4)N5)c3CO2)c1C(F)(F)F]

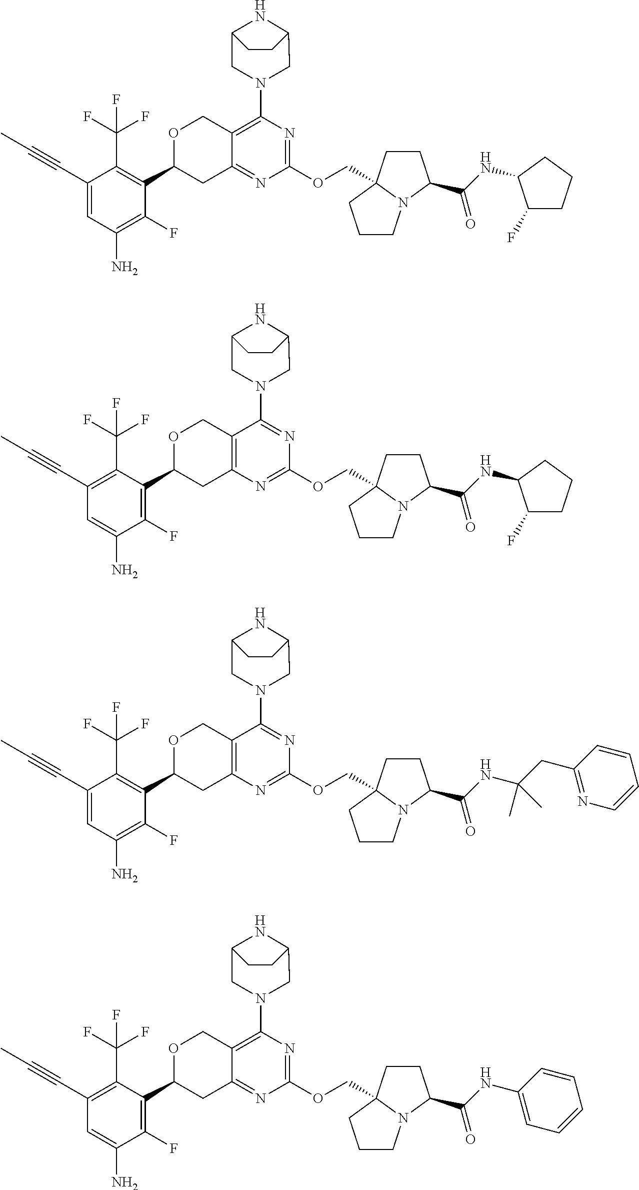 [SMILES: CC#Cc1cc(N)c(F)c([C@@H]2Cc3nc(OC[C@@]45CCCN4[C@H](C(=O)N[C@@H]4CCC[C@@H]4F)CC5)nc(N4CC5CCC(C4)N5)c3CO2)c1C(F)(F)F ; CC#Cc1cc(N)c(F)c([C@@H]2Cc3nc(OC[C@@]45CCCN4[C@H](C(=O)N[C@H]4CCC[C@@H]4F)CC5)nc(N4CC5CCC(C4)N5)c3CO2)c1C(F)(F)F ; CC#Cc1cc(N)c(F)c([C@@H]2Cc3nc(OC[C@@]45CCCN4[C@H](C(=O)NC(C)(C)Cc4ccccn4)CC5)nc(N4CC5CCC(C4)N5)c3CO2)c1C(F)(F)F ; CC#Cc1cc(N)c(F)c([C@@H]2Cc3nc(OC[C@@]45CCCN4[C@H](C(=O)Nc4ccccc4)CC5)nc(N4CC5CCC(C4)N5)c3CO2)c1C(F)(F)F]

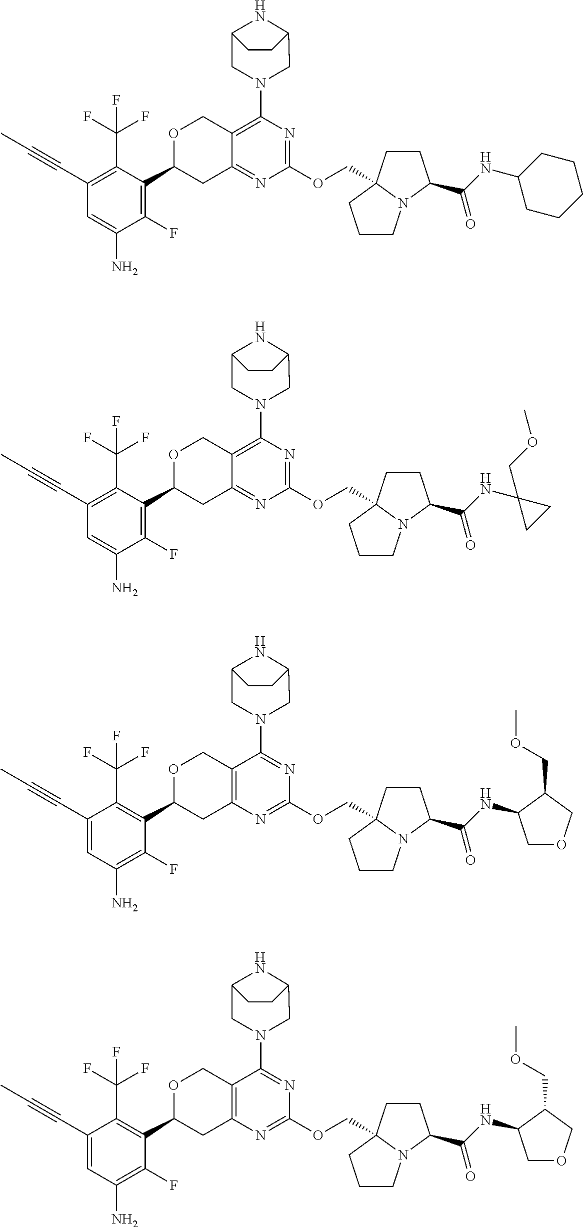 [SMILES: CC#Cc1cc(N)c(F)c([C@@H]2Cc3nc(OC[C@@]45CCCN4[C@H](C(=O)NC4CCCCC4)CC5)nc(N4CC5CCC(C4)N5)c3CO2)c1C(F)(F)F ; CC#Cc1cc(N)c(F)c([C@@H]2Cc3nc(OC[C@@]45CCCN4[C@H](C(=O)NC4(COC)CC4)CC5)nc(N4CC5CCC(C4)N5)c3CO2)c1C(F)(F)F ; CC#Cc1cc(N)c(F)c([C@@H]2Cc3nc(OC[C@@]45CCCN4[C@H](C(=O)N[C@@H]4COC[C@@H]4COC)CC5)nc(N4CC5CCC(C4)N5)c3CO2)c1C(F)(F)F ; CC#Cc1cc(N)c(F)c([C@@H]2Cc3nc(OC[C@@]45CCCN4[C@H](C(=O)N[C@@H]4COC[C@H]4COC)CC5)nc(N4CC5CCC(C4)N5)c3CO2)c1C(F)(F)F]

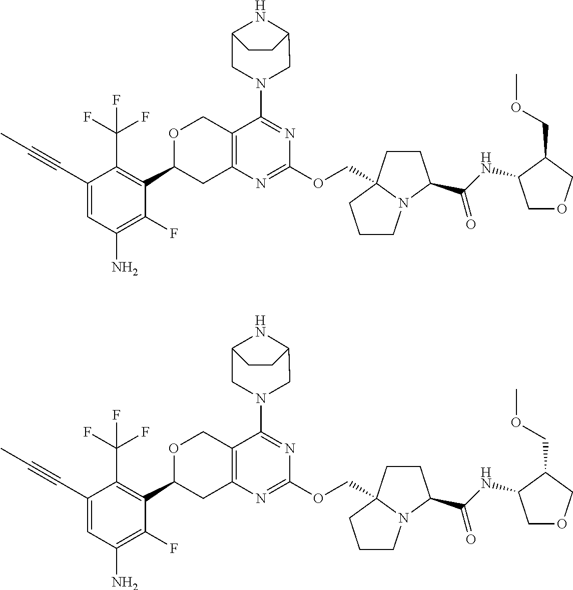 [SMILES: CC#Cc1cc(N)c(F)c([C@@H]2Cc3nc(OC[C@@]45CCCN4[C@H](C(=O)N[C@H]4COC[C@@H]4COC)CC5)nc(N4CC5CCC(C4)N5)c3CO2)c1C(F)(F)F ; CC#Cc1cc(N)c(F)c([C@@H]2Cc3nc(OC[C@@]45CCCN4[C@H](C(=O)N[C@H]4COC[C@H]4COC)CC5)nc(N4CC5CCC(C4)N5)c3CO2)c1C(F)(F)F]

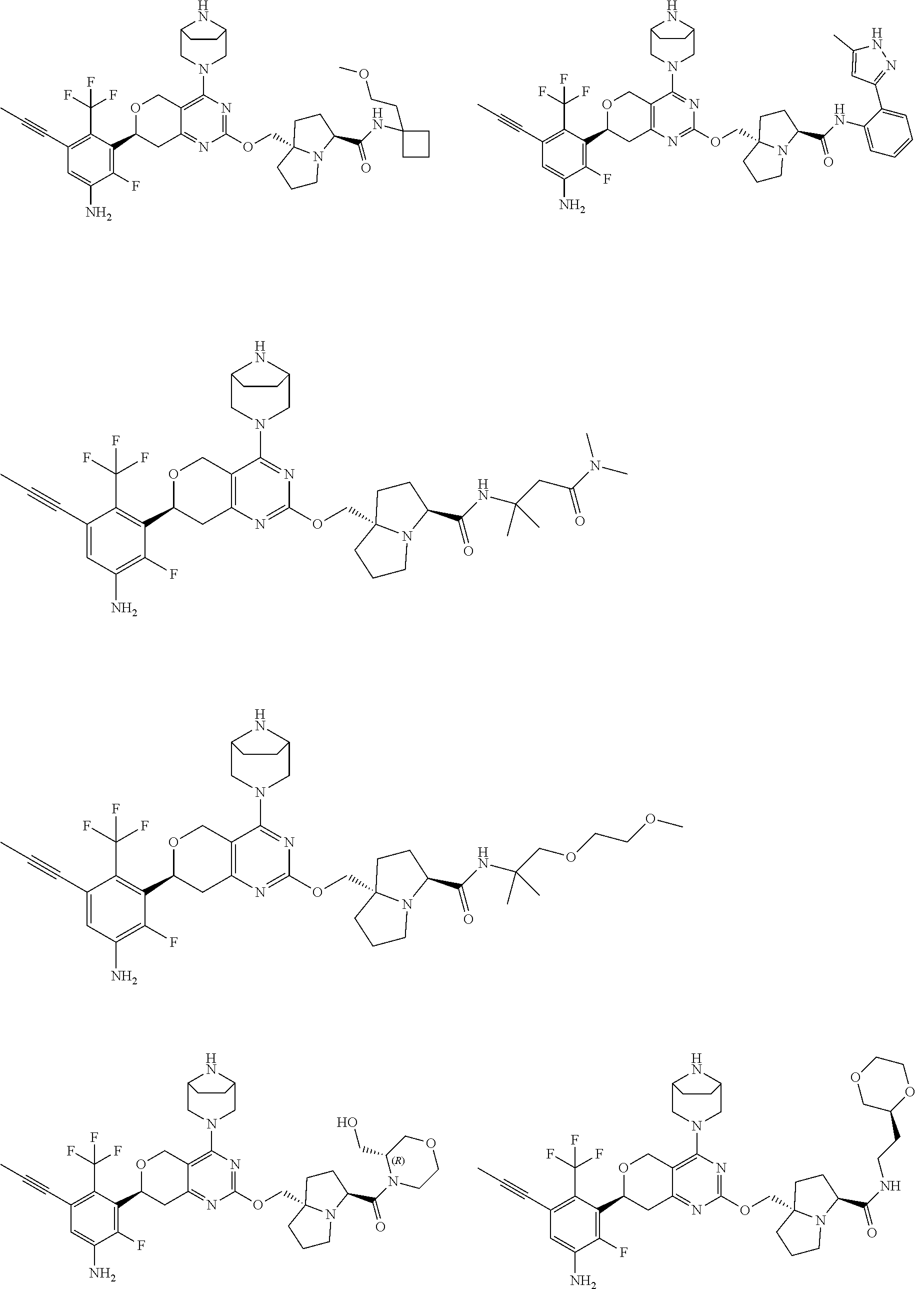

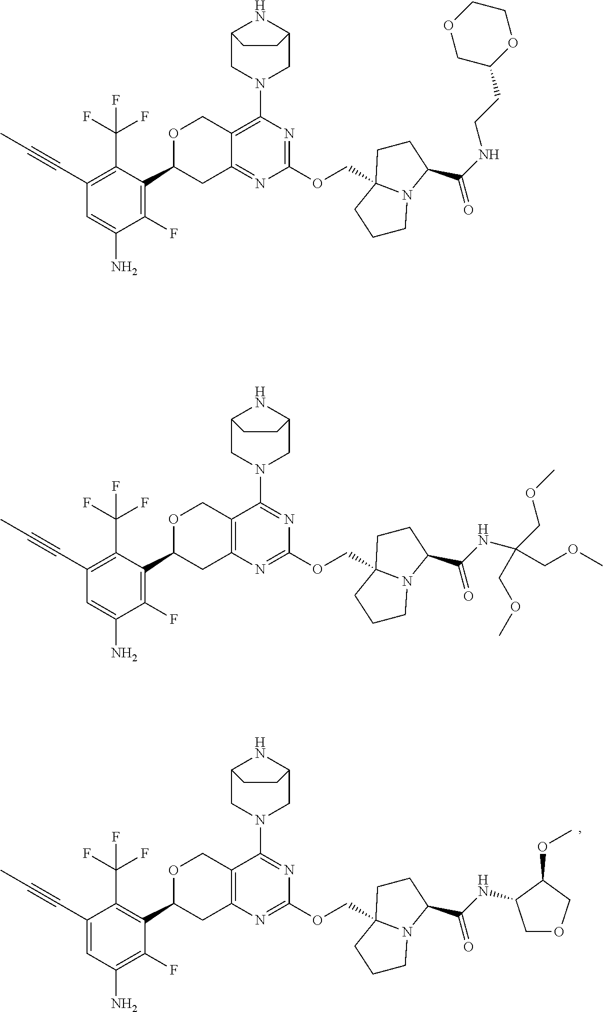

or a pharmaceutically acceptable salt thereof.

TABLE 5

| | |
|---|---|
| 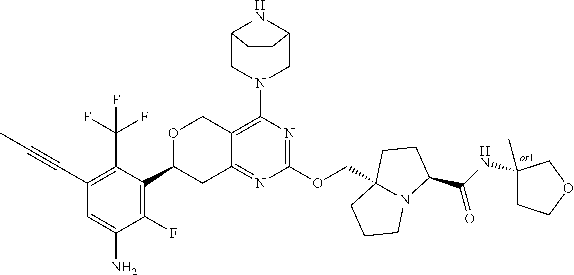 [SMILES: CC#Cc1cc(N)c(F)c([C@@H]2Cc3nc(OC[C@@]45CCCN4[C@H](C(=O)N[C@@]4(C)CCOC4)CC5)nc(N4CC5CCC(C4)N5)c3CO2)c1C(F)(F)F] | Example S1 |
| 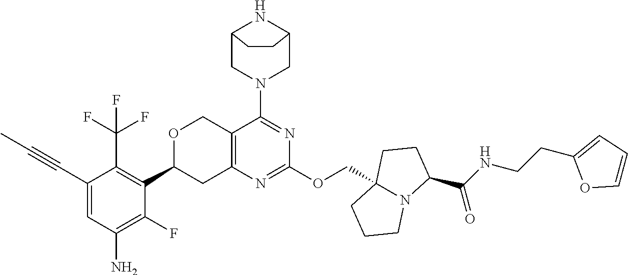 [SMILES: CC#Cc1cc(N)c(F)c([C@@H]2Cc3nc(OC[C@@]45CCCN4[C@H](C(=O)NCCc4ccco4)CC5)nc(N4CC5CCC(C4)N5)c3CO2)c1C(F)(F)F] | Example S2 |
| 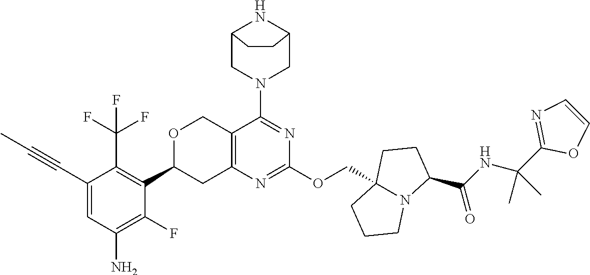 [SMILES: CC#Cc1cc(N)c(F)c([C@@H]2Cc3nc(OC[C@@]45CCCN4[C@H](C(=O)NC(C)(C)c4ncco4)CC5)nc(N4CC5CCC(C4)N5)c3CO2)c1C(F)(F)F] | Example S3 |
| 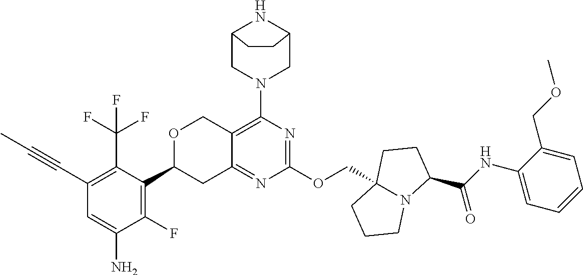 [SMILES: CC#Cc1cc(N)c(F)c([C@@H]2Cc3nc(OC[C@@]45CCCN4[C@H](C(=O)Nc4ccccc4COC)CC5)nc(N4CC5CCC(C4)N5)c3CO2)c1C(F)(F)F] | Example S4 |
| 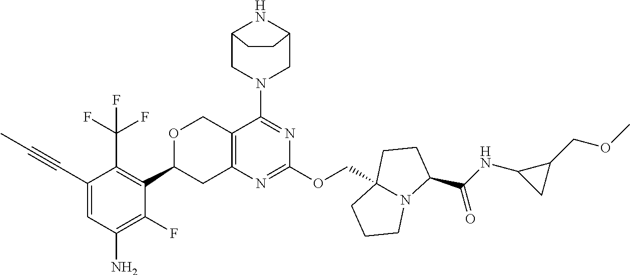 [SMILES: CC#Cc1cc(N)c(F)c([C@@H]2Cc3nc(OC[C@@]45CCCN4[C@H](C(=O)NC4CC4COC)CC5)nc(N4CC5CCC(C4)N5)c3CO2)c1C(F)(F)F] | Example S5 |
| (mix of 4 isomers) | |
| 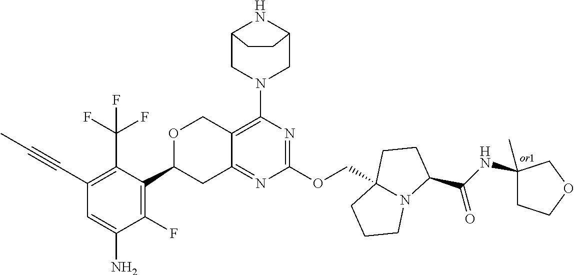 [SMILES: CC#Cc1cc(N)c(F)c([C@@H]2Cc3nc(OC[C@@]45CCCN4[C@H](C(=O)N[C@]4(C)CCOC4)CC5)nc(N4CC5CCC(C4)N5)c3CO2)c1C(F)(F)F] | Example S6 |
| 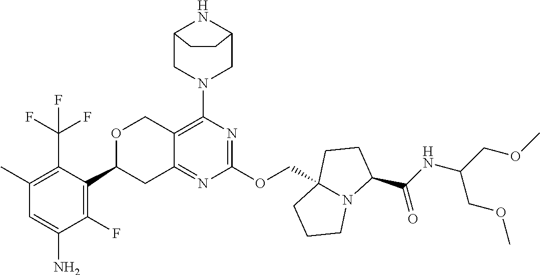 | Example S7 |
| 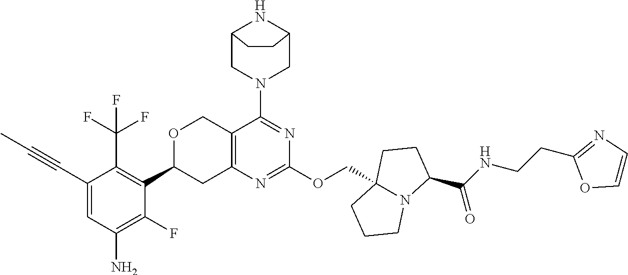 [SMILES: CC#Cc1cc(N)c(F)c([C@@H]2Cc3nc(OC[C@@]45CCCN4[C@H](C(=O)NCCc4ncco4)CC5)nc(N4CC5CCC(C4)N5)c3CO2)c1C(F)(F)F] | Example S8 |
| 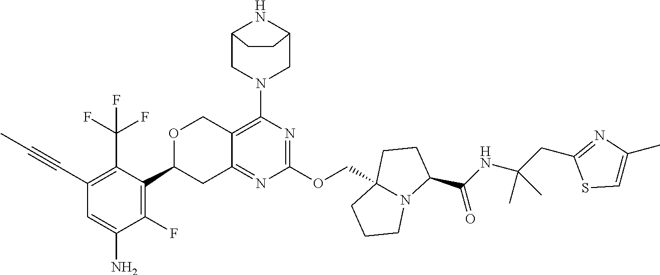 [SMILES: CC#Cc1cc(N)c(F)c([C@@H]2Cc3nc(OC[C@@]45CCCN4[C@H](C(=O)NC(C)(C)Cc4nc(C)cs4)CC5)nc(N4CC5CCC(C4)N5)c3CO2)c1C(F)(F)F] | Example S9 |
| 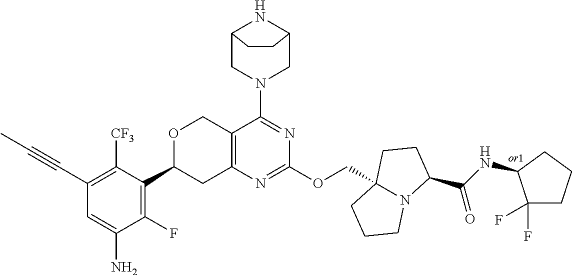 [SMILES: CC#Cc1cc(N)c(F)c([C@@H]2Cc3nc(OC[C@@]45CCCN4[C@H](C(=O)N[C@H]4CCCC4(F)F)CC5)nc(N4CC5CCC(C4)N5)c3CO2)c1C(F)(F)F] | Example S10 |
| 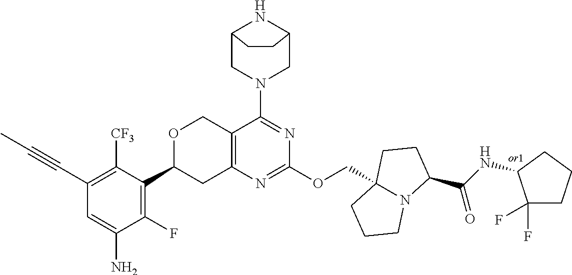 [SMILES: CC#Cc1cc(N)c(F)c([C@@H]2Cc3nc(OC[C@@]45CCCN4[C@H](C(=O)N[C@@H]4CCCC4(F)F)CC5)nc(N4CC5CCC(C4)N5)c3CO2)c1C(F)(F)F] | Example S11 |
| 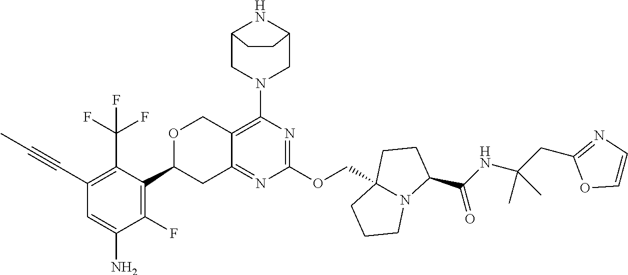 [SMILES: CC#Cc1cc(N)c(F)c([C@@H]2Cc3nc(OC[C@@]45CCCN4[C@H](C(=O)NC(C)(C)Cc4ncco4)CC5)nc(N4CC5CCC(C4)N5)c3CO2)c1C(F)(F)F] | Example S12 |
| 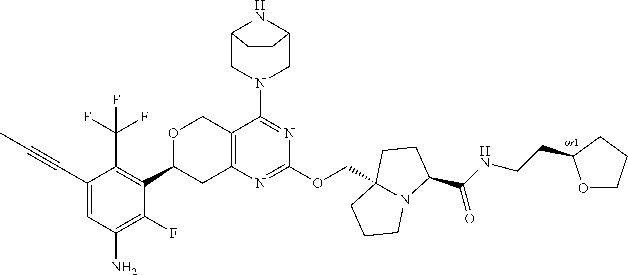 [SMILES: CC#Cc1cc(N)c(F)c([C@@H]2Cc3nc(OC[C@@]45CCCN4[C@H](C(=O)NCC[C@H]4CCCO4)CC5)nc(N4CC5CCC(C4)N5)c3CO2)c1C(F)(F)F] | Example S13 |
| 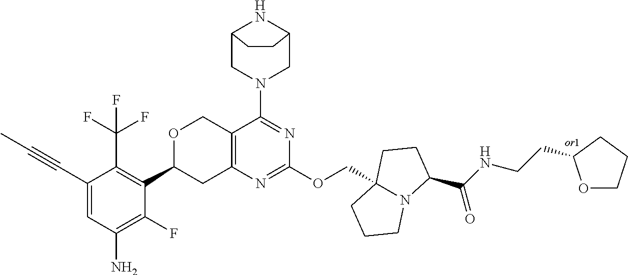 [SMILES: CC#Cc1cc(N)c(F)c([C@@H]2Cc3nc(OC[C@@]45CCCN4[C@H](C(=O)NCC[C@@H]4CCCO4)CC5)nc(N4CC5CCC(C4)N5)c3CO2)c1C(F)(F)F] | Example S14 |
| 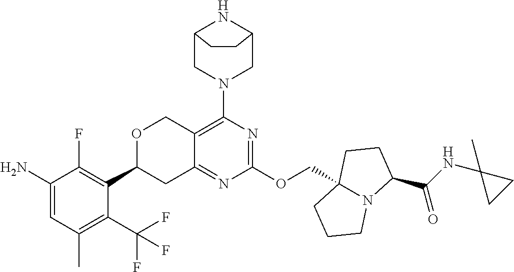 [SMILES: Cc1cc(N)c(F)c([C@@H]2Cc3nc(OC[C@@]45CCCN4[C@H](C(=O)NC4(C)CC4)CC5)nc(N4CC5CCC(C4)N5)c3CO2)c1C(F)(F)F] | Example S15 |
| 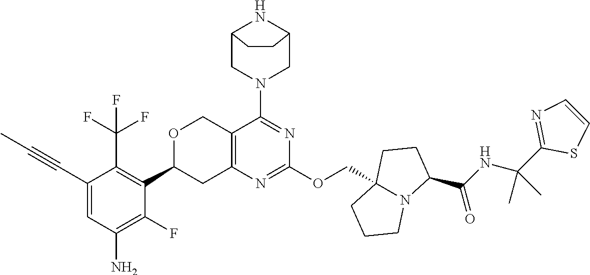 [SMILES: CC#Cc1cc(N)c(F)c([C@@H]2Cc3nc(OC[C@@]45CCCN4[C@H](C(=O)NC(C)(C)c4nccs4)CC5)nc(N4CC5CCC(C4)N5)c3CO2)c1C(F)(F)F] | Example S16 |
| 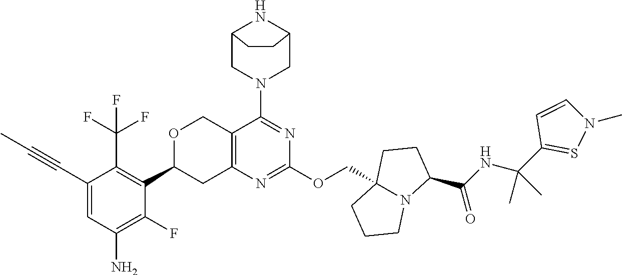 | Example S17 |
| 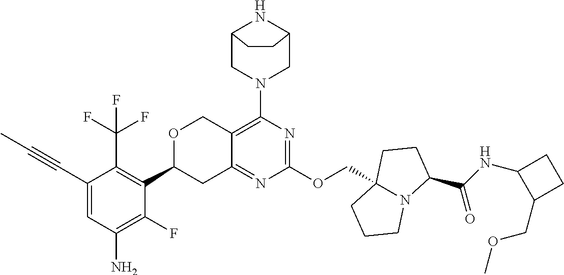 [SMILES: CC#Cc1cc(N)c(F)c([C@@H]2Cc3nc(OC[C@@]45CCCN4[C@H](C(=O)NC4CCC4COC)CC5)nc(N4CC5CCC(C4)N5)c3CO2)c1C(F)(F)F] | Example S18A/S18B |
| 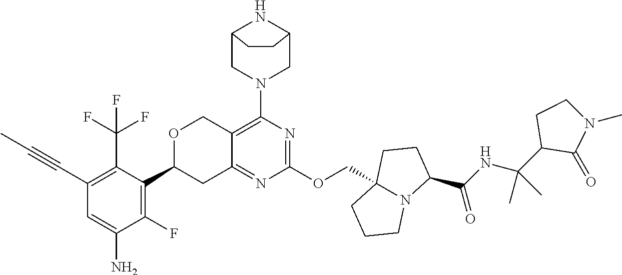 [SMILES: CC#Cc1cc(N)c(F)c([C@@H]2Cc3nc(OC[C@@]45CCCN4[C@H](C(=O)NC(C)(C)C4CCN(C)C4=O)CC5)nc(N4CC5CCC(C4)N5)c3CO2)c1C(F)(F)F] | Example S19A/S19B |
| 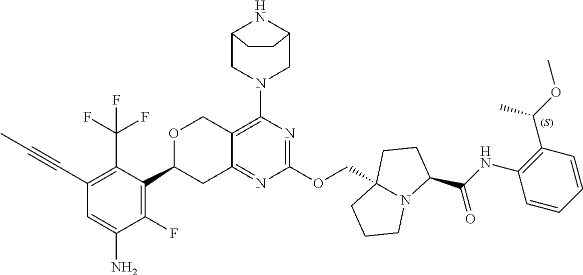 | Example S20 |
| 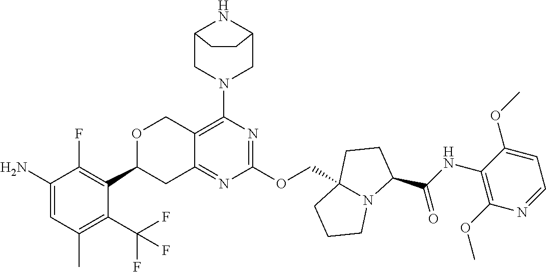 [SMILES: COc1ccnc(OC)c1NC(=O)[C@@H]1CC[C@]2(COc3nc4c(c(N5CC6CCC(C5)N6)n3)CO[C@H](c3c(F)c(N)cc(C)c3C(F)(F)F)C4)CCCN12] | Example S21 |
| 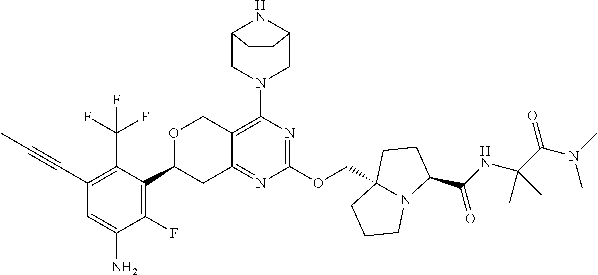 [SMILES: CC#Cc1cc(N)c(F)c([C@@H]2Cc3nc(OC[C@@]45CCCN4[C@H](C(=O)NC(C)(C)C(=O)N(C)C)CC5)nc(N4CC5CCC(C4)N5)c3CO2)c1C(F)(F)F] | Example S22 |
| 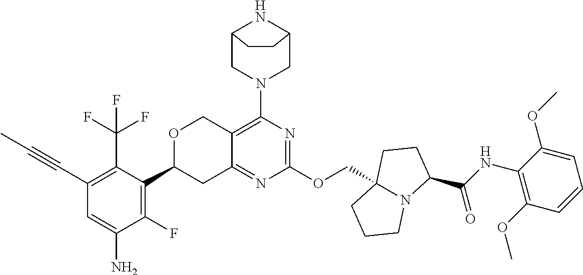 [SMILES: CC#Cc1cc(N)c(F)c([C@@H]2Cc3nc(OC[C@@]45CCCN4[C@H](C(=O)Nc4c(OC)cccc4OC)CC5)nc(N4CC5CCC(C4)N5)c3CO2)c1C(F)(F)F] | Example S23 |
| 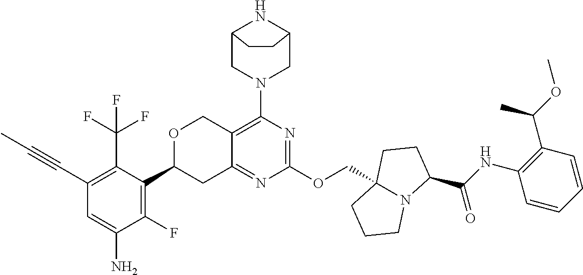 [SMILES: CC#Cc1cc(N)c(F)c([C@@H]2Cc3nc(OC[C@@]45CCCN4[C@H](C(=O)Nc4ccccc4[C@@H](C)OC)CC5)nc(N4CC5CCC(C4)N5)c3CO2)c1C(F)(F)F] | Example S24 |
| 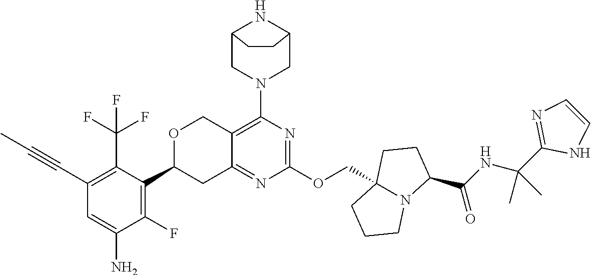 [SMILES: CC#Cc1cc(N)c(F)c([C@@H]2Cc3nc(OC[C@@]45CCCN4[C@H](C(=O)NC(C)(C)c4ncc[nH]4)CC5)nc(N4CC5CCC(C4)N5)c3CO2)c1C(F)(F)F] | Example S25 |
| 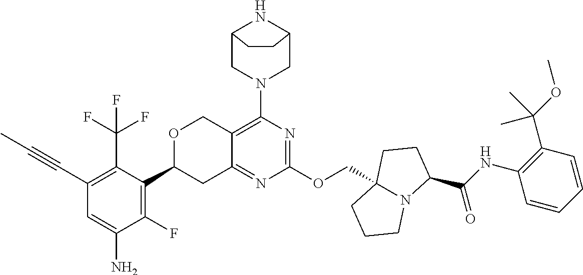 [SMILES: CC#Cc1cc(N)c(F)c([C@@H]2Cc3nc(OC[C@@]45CCCN4[C@H](C(=O)Nc4ccccc4C(C)(C)OC)CC5)nc(N4CC5CCC(C4)N5)c3CO2)c1C(F)(F)F] | Example S26 |
| 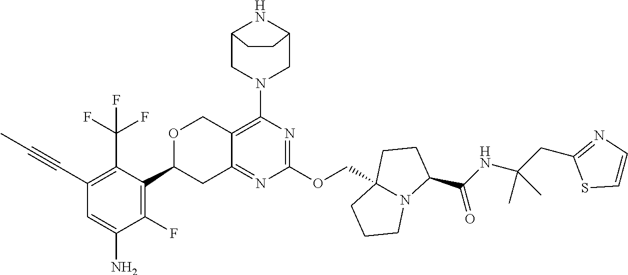 [SMILES: CC#Cc1cc(N)c(F)c([C@@H]2Cc3nc(OC[C@@]45CCCN4[C@H](C(=O)NC(C)(C)Cc4nccs4)CC5)nc(N4CC5CCC(C4)N5)c3CO2)c1C(F)(F)F] | Example S27 |
| 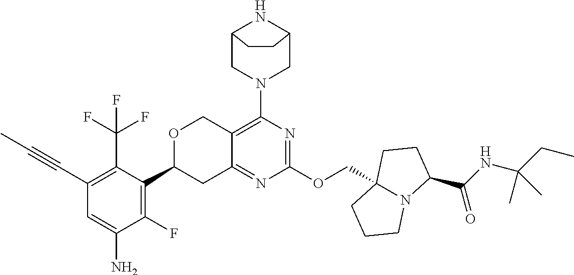 [SMILES: CC#Cc1cc(N)c(F)c([C@@H]2Cc3nc(OC[C@@]45CCCN4[C@H](C(=O)NC(C)(C)CC)CC5)nc(N4CC5CCC(C4)N5)c3CO2)c1C(F)(F)F] | Example S28 |
| 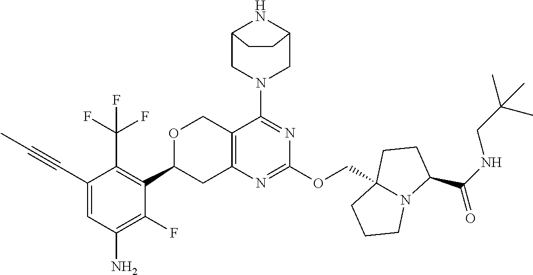 [SMILES: CC#Cc1cc(N)c(F)c([C@@H]2Cc3nc(OC[C@@]45CCCN4[C@H](C(=O)NCC(C)(C)C)CC5)nc(N4CC5CCC(C4)N5)c3CO2)c1C(F)(F)F] | Example S29 |
| 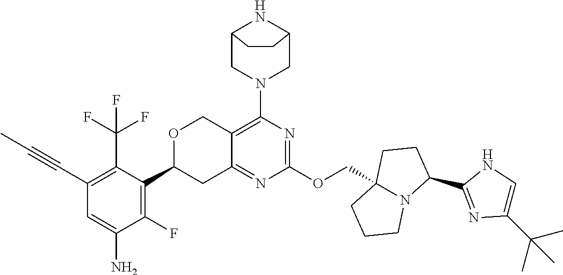 [SMILES: CC#Cc1cc(N)c(F)c([C@@H]2Cc3nc(OC[C@@]45CCCN4[C@H](c4nc(C(C)(C)C)c[nH]4)CC5)nc(N4CC5CCC(C4)N5)c3CO2)c1C(F)(F)F] | Example S30 |
| 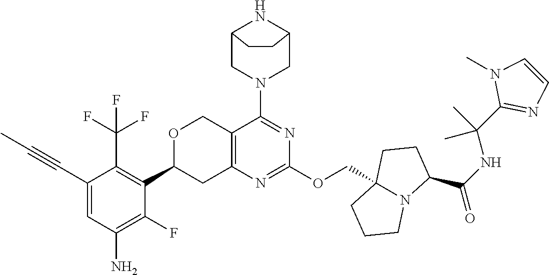 [SMILES: CC#Cc1cc(N)c(F)c([C@@H]2Cc3nc(OC[C@@]45CCCN4[C@H](C(=O)NC(C)(C)c4nccn4C)CC5)nc(N4CC5CCC(C4)N5)c3CO2)c1C(F)(F)F] | Example S31 |
| 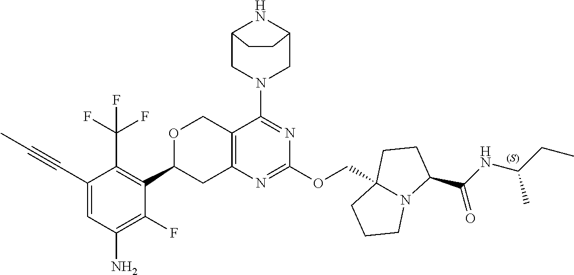 | Example S32 |
| 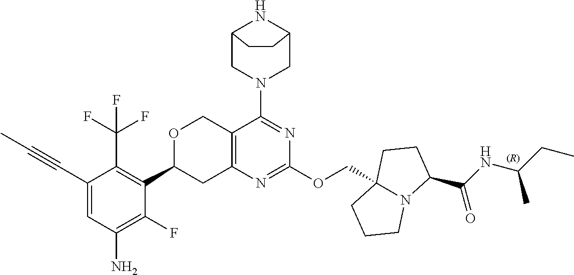 | Example S33 |
| 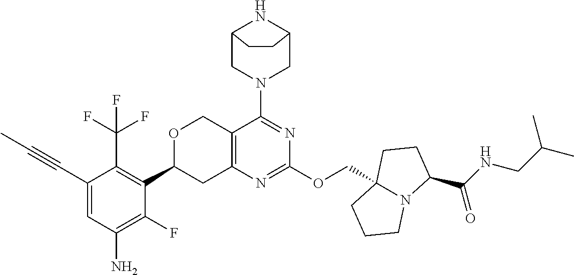 [SMILES: CC#Cc1cc(N)c(F)c([C@@H]2Cc3nc(OC[C@@]45CCCN4[C@H](C(=O)NCC(C)C)CC5)nc(N4CC5CCC(C4)N5)c3CO2)c1C(F)(F)F] | Example S34 |
| 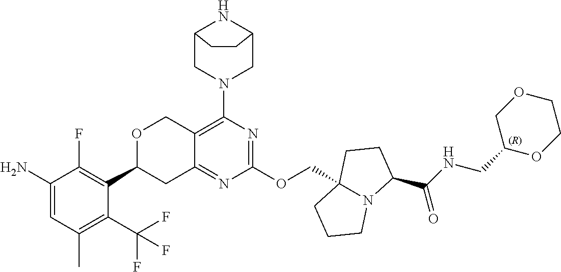 | Example S35 |
| 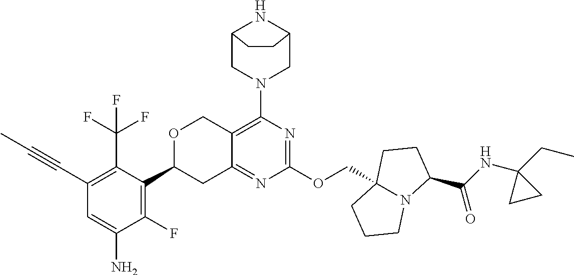 [SMILES: CC#Cc1cc(N)c(F)c([C@@H]2Cc3nc(OC[C@@]45CCCN4[C@H](C(=O)NC4(CC)CC4)CC5)nc(N4CC5CCC(C4)N5)c3CO2)c1C(F)(F)F] | Example S36 |
| 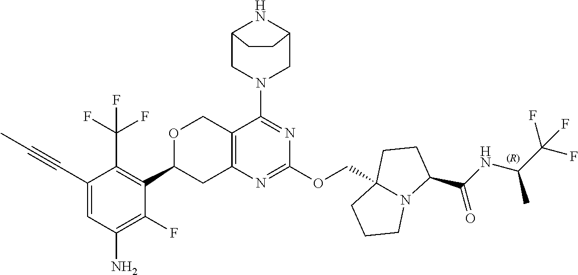 | Example S37 |
| 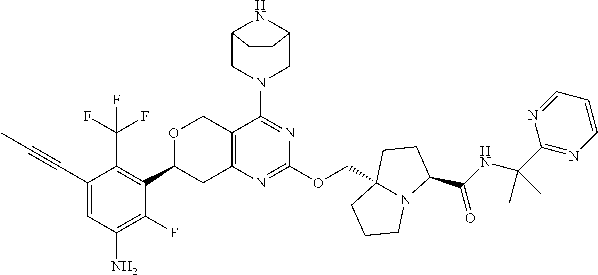 [SMILES: CC#Cc1cc(N)c(F)c([C@@H]2Cc3nc(OC[C@@]45CCCN4[C@H](C(=O)NC(C)(C)c4ncccn4)CC5)nc(N4CC5CCC(C4)N5)c3CO2)c1C(F)(F)F] | Example S38 |
| 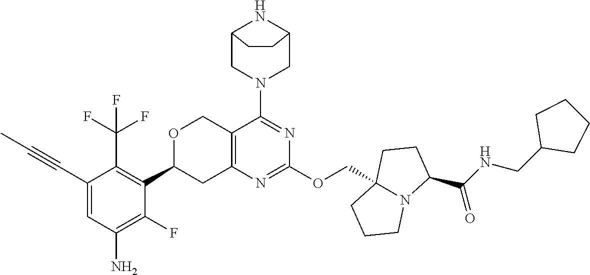 [SMILES: CC#Cc1cc(N)c(F)c([C@@H]2Cc3nc(OC[C@@]45CCCN4[C@H](C(=O)NCC4CCCC4)CC5)nc(N4CC5CCC(C4)N5)c3CO2)c1C(F)(F)F] | Example S39 |
| 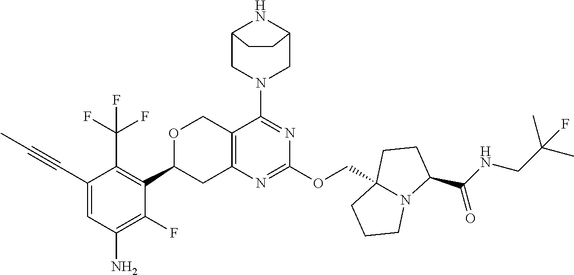 [SMILES: CC#Cc1cc(N)c(F)c([C@@H]2Cc3nc(OC[C@@]45CCCN4[C@H](C(=O)NCC(C)(C)F)CC5)nc(N4CC5CCC(C4)N5)c3CO2)c1C(F)(F)F] | Example S40 |
| 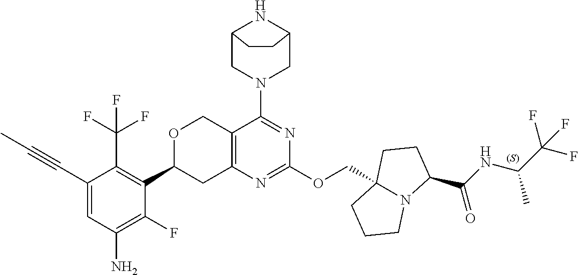 | Example S41 |
| 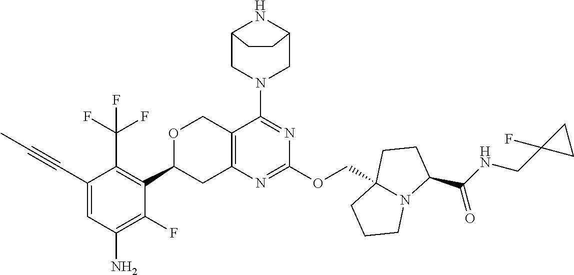 [SMILES: CC#Cc1cc(N)c(F)c([C@@H]2Cc3nc(OC[C@@]45CCCN4[C@H](C(=O)NCC4(F)CC4)CC5)nc(N4CC5CCC(C4)N5)c3CO2)c1C(F)(F)F] | Example S42 |
| 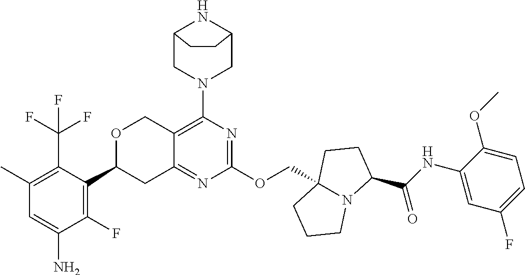 [SMILES: COc1ccc(F)cc1NC(=O)[C@@H]1CC[C@]2(COc3nc4c(c(N5CC6CCC(C5)N6)n3)CO[C@H](c3c(F)c(N)cc(C)c3C(F)(F)F)C4)CCCN12] | Example S43 |
| 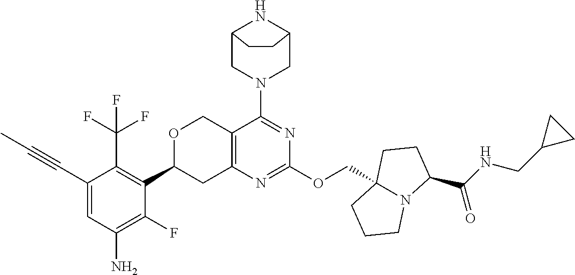 [SMILES: CC#Cc1cc(N)c(F)c([C@@H]2Cc3nc(OC[C@@]45CCCN4[C@H](C(=O)NCC4CC4)CC5)nc(N4CC5CCC(C4)N5)c3CO2)c1C(F)(F)F] | Example S44 |
| 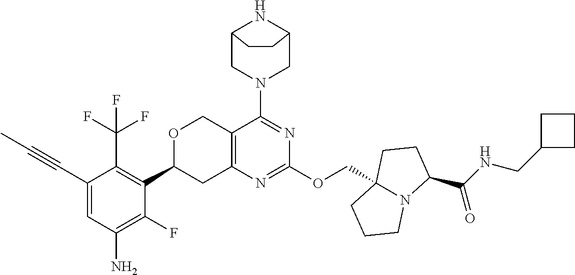 [SMILES: CC#Cc1cc(N)c(F)c([C@@H]2Cc3nc(OC[C@@]45CCCN4[C@H](C(=O)NCC4CCC4)CC5)nc(N4CC5CCC(C4)N5)c3CO2)c1C(F)(F)F] | Example S45 |
| 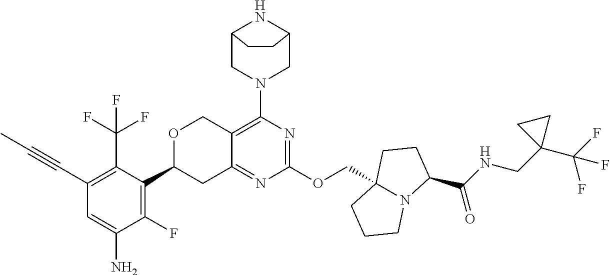 [SMILES: CC#Cc1cc(N)c(F)c([C@@H]2Cc3nc(OC[C@@]45CCCN4[C@H](C(=O)NCC4(C(F)(F)F)CC4)CC5)nc(N4CC5CCC(C4)N5)c3CO2)c1C(F)(F)F] | Example S46 |
| 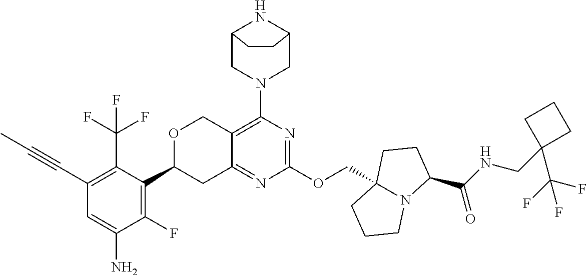 [SMILES: CC#Cc1cc(N)c(F)c([C@@H]2Cc3nc(OC[C@@]45CCCN4[C@H](C(=O)NCC4(C(F)(F)F)CCC4)CC5)nc(N4CC5CCC(C4)N5)c3CO2)c1C(F)(F)F] | Example S47 |
| 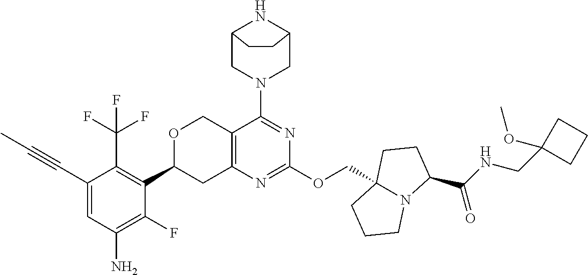 [SMILES: CC#Cc1cc(N)c(F)c([C@@H]2Cc3nc(OC[C@@]45CCCN4[C@H](C(=O)NCC4(OC)CCC4)CC5)nc(N4CC5CCC(C4)N5)c3CO2)c1C(F)(F)F] | Example S48 |
| 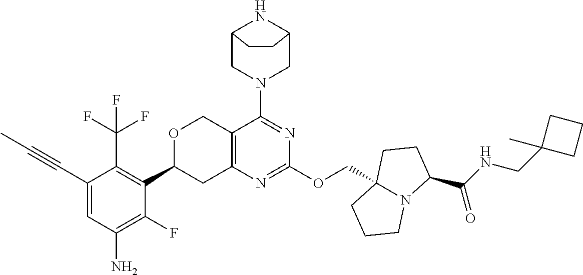 [SMILES: CC#Cc1cc(N)c(F)c([C@@H]2Cc3nc(OC[C@@]45CCCN4[C@H](C(=O)NCC4(C)CCC4)CC5)nc(N4CC5CCC(C4)N5)c3CO2)c1C(F)(F)F] | Example S49 |
| 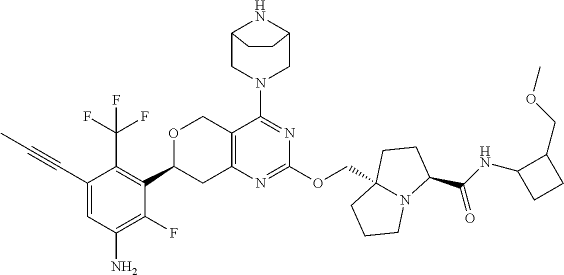 [SMILES: CC#Cc1cc(N)c(F)c([C@@H]2Cc3nc(OC[C@@]45CCCN4[C@H](C(=O)NC4CCC4COC)CC5)nc(N4CC5CCC(C4)N5)c3CO2)c1C(F)(F)F] | Example S50A/S50B |
| 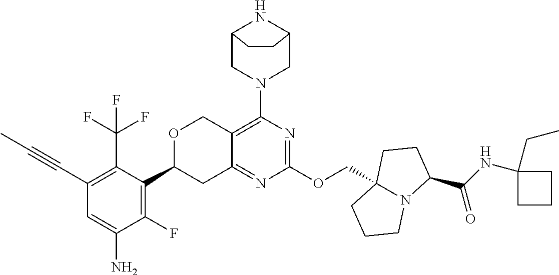 [SMILES: CC#Cc1cc(N)c(F)c([C@@H]2Cc3nc(OC[C@@]45CCCN4[C@H](C(=O)NC4(CC)CCC4)CC5)nc(N4CC5CCC(C4)N5)c3CO2)c1C(F)(F)F] | Example S51 |
| 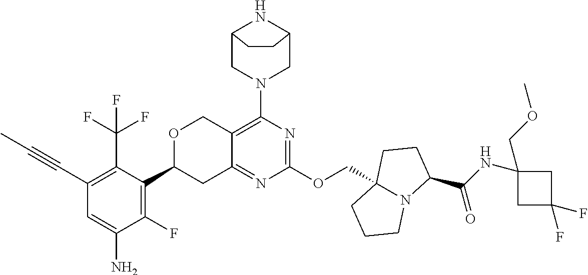 [SMILES: CC#Cc1cc(N)c(F)c([C@@H]2Cc3nc(OC[C@@]45CCCN4[C@H](C(=O)NC4(COC)CC(F)(F)C4)CC5)nc(N4CC5CCC(C4)N5)c3CO2)c1C(F)(F)F] | Example S52 |
| 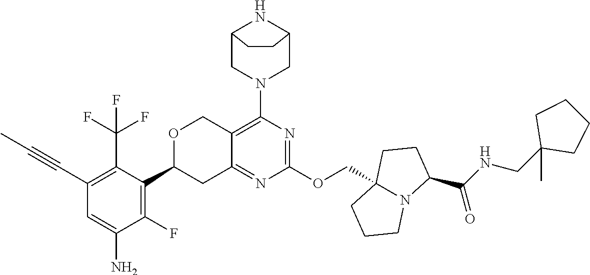 [SMILES: CC#Cc1cc(N)c(F)c([C@@H]2Cc3nc(OC[C@@]45CCCN4[C@H](C(=O)NCC4(C)CCCC4)CC5)nc(N4CC5CCC(C4)N5)c3CO2)c1C(F)(F)F] | Example S53 |
| 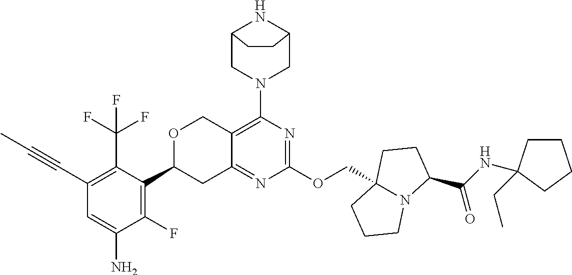 [SMILES: CC#Cc1cc(N)c(F)c([C@@H]2Cc3nc(OC[C@@]45CCCN4[C@H](C(=O)NC4(CC)CCCC4)CC5)nc(N4CC5CCC(C4)N5)c3CO2)c1C(F)(F)F] | Example S54 |
| 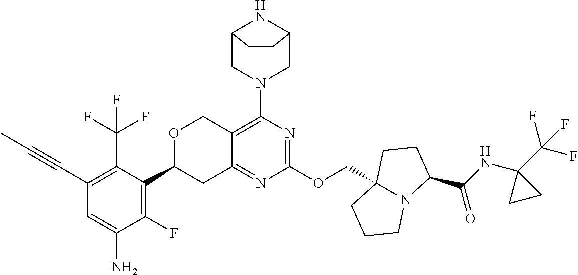 [SMILES: CC#Cc1cc(N)c(F)c([C@@H]2Cc3nc(OC[C@@]45CCCN4[C@H](C(=O)NC4(C(F)(F)F)CC4)CC5)nc(N4CC5CCC(C4)N5)c3CO2)c1C(F)(F)F] | Example S55 |
| 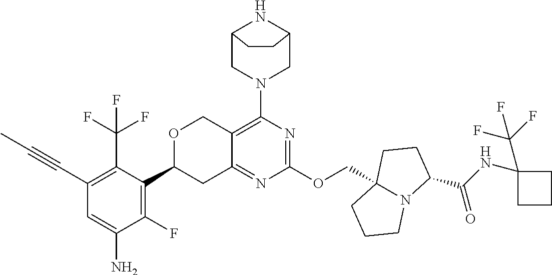 [SMILES: CC#Cc1cc(N)c(F)c([C@@H]2Cc3nc(OC[C@@]45CCCN4[C@@H](C(=O)NC4(C(F)(F)F)CCC4)CC5)nc(N4CC5CCC(C4)N5)c3CO2)c1C(F)(F)F] | Example S56 |
| 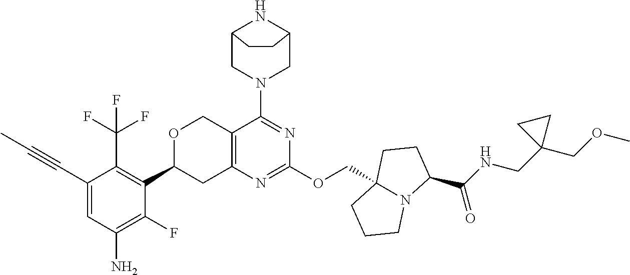 [SMILES: CC#Cc1cc(N)c(F)c([C@@H]2Cc3nc(OC[C@@]45CCCN4[C@H](C(=O)NCC4(COC)CC4)CC5)nc(N4CC5CCC(C4)N5)c3CO2)c1C(F)(F)F] | Example S57 |
| 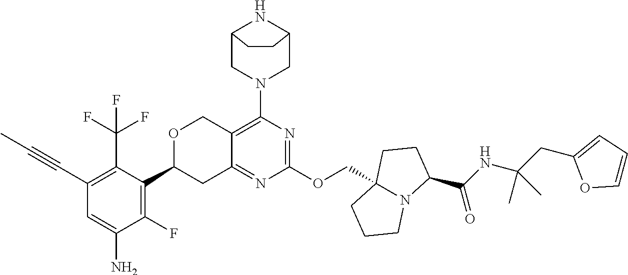 [SMILES: CC#Cc1cc(N)c(F)c([C@@H]2Cc3nc(OC[C@@]45CCCN4[C@H](C(=O)NC(C)(C)Cc4ccco4)CC5)nc(N4CC5CCC(C4)N5)c3CO2)c1C(F)(F)F] | Example S58 |
| 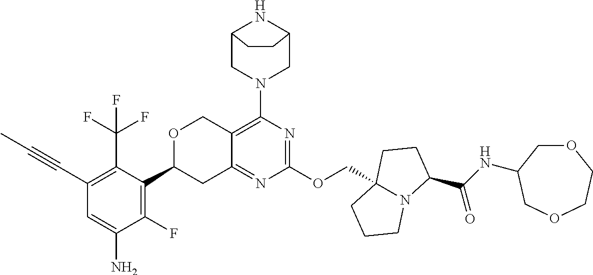 [SMILES: CC#Cc1cc(N)c(F)c([C@@H]2Cc3nc(OC[C@@]45CCCN4[C@H](C(=O)NC4COCCOC4)CC5)nc(N4CC5CCC(C4)N5)c3CO2)c1C(F)(F)F] | Example S59 |
| 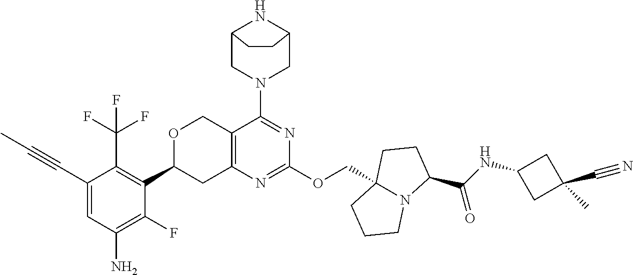 [SMILES: CC#Cc1cc(N)c(F)c([C@@H]2Cc3nc(OC[C@@]45CCCN4[C@H](C(=O)N[C@H]4C[C@](C)(C#N)C4)CC5)nc(N4CC5CCC(C4)N5)c3CO2)c1C(F)(F)F] | Example S60 |
| 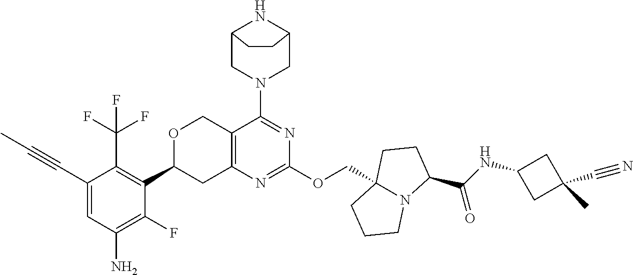 [SMILES: CC#Cc1cc(N)c(F)c([C@@H]2Cc3nc(OC[C@@]45CCCN4[C@H](C(=O)N[C@H]4C[C@@](C)(C#N)C4)CC5)nc(N4CC5CCC(C4)N5)c3CO2)c1C(F)(F)F] | Example S61 |
| 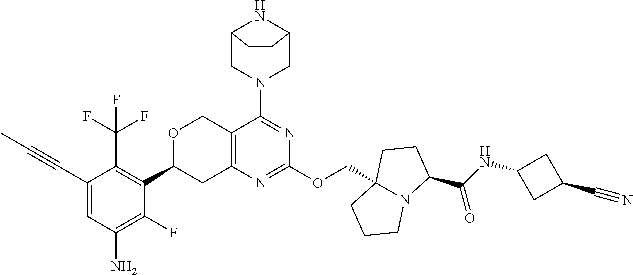 [SMILES: CC#Cc1cc(N)c(F)c([C@@H]2Cc3nc(OC[C@@]45CCCN4[C@H](C(=O)N[C@H]4C[C@H](C#N)C4)CC5)nc(N4CC5CCC(C4)N5)c3CO2)c1C(F)(F)F] | Example S62 |
| 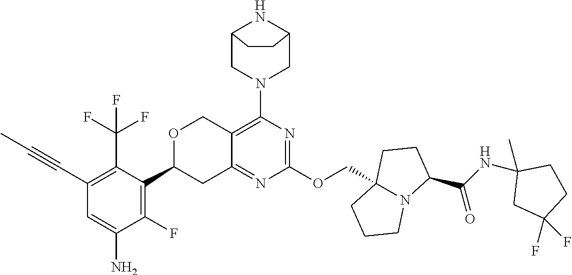 [SMILES: CC#Cc1cc(N)c(F)c([C@@H]2Cc3nc(OC[C@@]45CCCN4[C@H](C(=O)NC4(C)CCC(F)(F)C4)CC5)nc(N4CC5CCC(C4)N5)c3CO2)c1C(F)(F)F] | Example S63A/S63B |
| 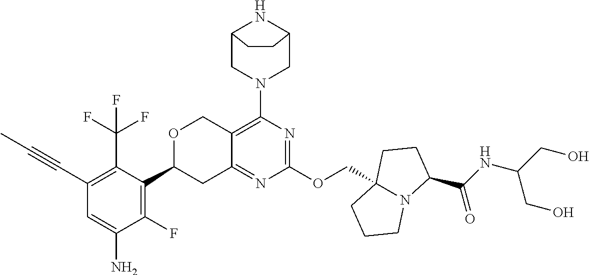 [SMILES: CC#Cc1cc(N)c(F)c([C@@H]2Cc3nc(OC[C@@]45CCCN4[C@H](C(=O)NC(CO)CO)CC5)nc(N4CC5CCC(C4)N5)c3CO2)c1C(F)(F)F] | Example S64 |
| 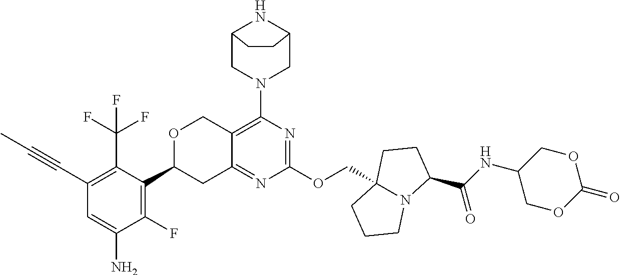 [SMILES: CC#Cc1cc(N)c(F)c([C@@H]2Cc3nc(OC[C@@]45CCCN4[C@H](C(=O)NC4COC(=O)OC4)CC5)nc(N4CC5CCC(C4)N5)c3CO2)c1C(F)(F)F] | Example S65 |
| 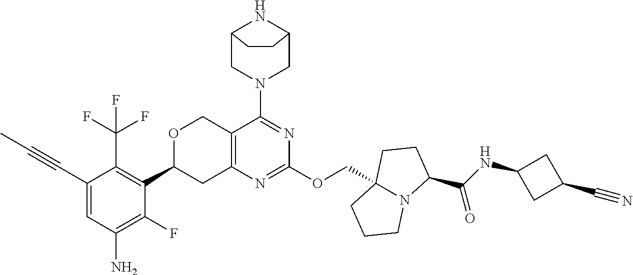 [SMILES: CC#Cc1cc(N)c(F)c([C@@H]2Cc3nc(OC[C@@]45CCCN4[C@H](C(=O)N[C@H]4C[C@@H](C#N)C4)CC5)nc(N4CC5CCC(C4)N5)c3CO2)c1C(F)(F)F] | Example S66 |
| 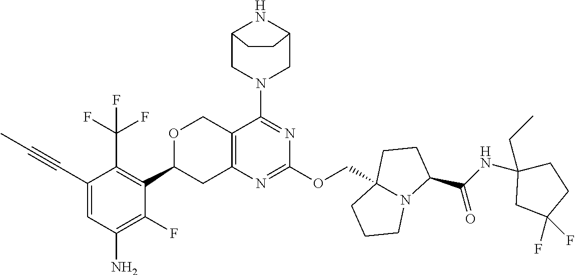 [SMILES: CC#Cc1cc(N)c(F)c([C@@H]2Cc3nc(OC[C@@]45CCCN4[C@H](C(=O)NC4(CC)CCC(F)(F)C4)CC5)nc(N4CC5CCC(C4)N5)c3CO2)c1C(F)(F)F] | Example S67A/S67B |
| 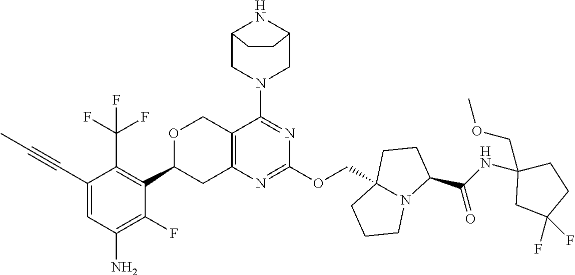 [SMILES: CC#Cc1cc(N)c(F)c([C@@H]2Cc3nc(OC[C@@]45CCCN4[C@H](C(=O)NC4(COC)CCC(F)(F)C4)CC5)nc(N4CC5CCC(C4)N5)c3CO2)c1C(F)(F)F] | Example S68A/S68B |
| 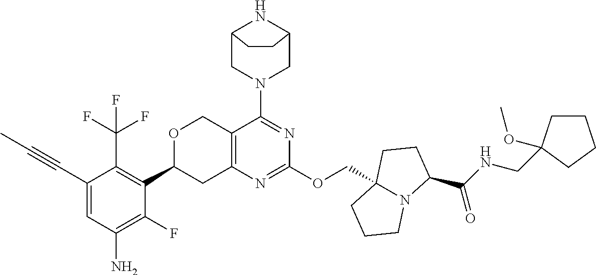 [SMILES: CC#Cc1cc(N)c(F)c([C@@H]2Cc3nc(OC[C@@]45CCCN4[C@H](C(=O)NCC4(OC)CCCC4)CC5)nc(N4CC5CCC(C4)N5)c3CO2)c1C(F)(F)F] | Example S69 |
| 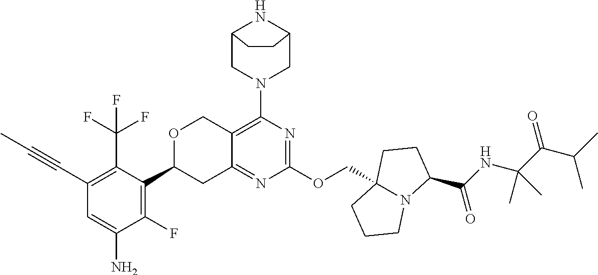 [SMILES: CC#Cc1cc(N)c(F)c([C@@H]2Cc3nc(OC[C@@]45CCCN4[C@H](C(=O)NC(C)(C)C(=O)C(C)C)CC5)nc(N4CC5CCC(C4)N5)c3CO2)c1C(F)(F)F] | Example S70 |
| 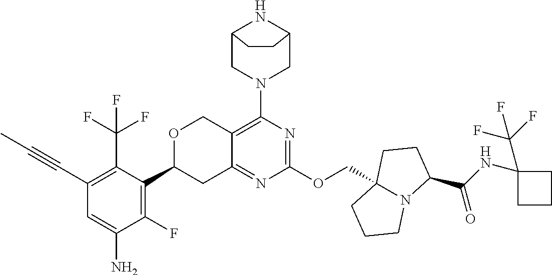 [SMILES: CC#Cc1cc(N)c(F)c([C@@H]2Cc3nc(OC[C@@]45CCCN4[C@H](C(=O)NC4(C(F)(F)F)CCC4)CC5)nc(N4CC5CCC(C4)N5)c3CO2)c1C(F)(F)F] | Example S71 |
| 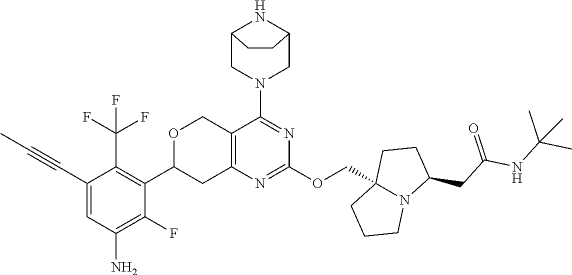 [SMILES: CC#Cc1cc(N)c(F)c(C2Cc3nc(OC[C@@]45CCCN4[C@H](CC(=O)NC(C)(C)C)CC5)nc(N4CC5CCC(C4)N5)c3CO2)c1C(F)(F)F] | Example S72 |
| 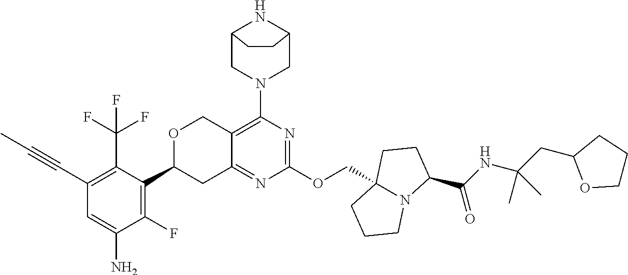 [SMILES: CC#Cc1cc(N)c(F)c([C@@H]2Cc3nc(OC[C@@]45CCCN4[C@H](C(=O)NC(C)(C)CC4CCCO4)CC5)nc(N4CC5CCC(C4)N5)c3CO2)c1C(F)(F)F] | Example S73A/S73B |
| 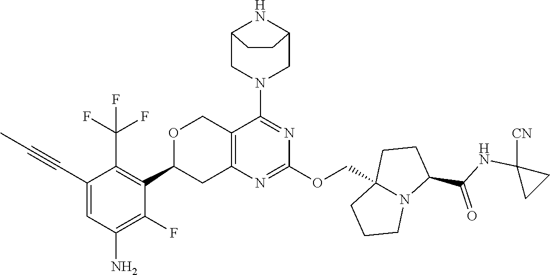 [SMILES: CC#Cc1cc(N)c(F)c([C@@H]2Cc3nc(OC[C@@]45CCCN4[C@H](C(=O)NC4(C#N)CC4)CC5)nc(N4CC5CCC(C4)N5)c3CO2)c1C(F)(F)F] | Example S74 |
| 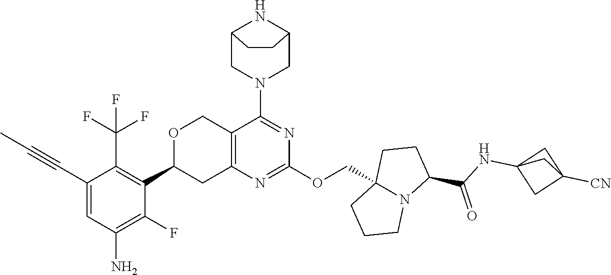 [SMILES: CC#Cc1cc(N)c(F)c([C@@H]2Cc3nc(OC[C@@]45CCCN4[C@H](C(=O)NC46CC(C#N)(C4)C6)CC5)nc(N4CC5CCC(C4)N5)c3CO2)c1C(F)(F)F] | Example S75 |
| 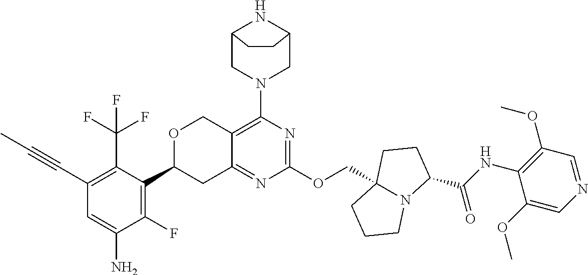 [SMILES: CC#Cc1cc(N)c(F)c([C@@H]2Cc3nc(OC[C@@]45CCCN4[C@@H](C(=O)Nc4c(OC)cncc4OC)CC5)nc(N4CC5CCC(C4)N5)c3CO2)c1C(F)(F)F] | Example S76 |
| 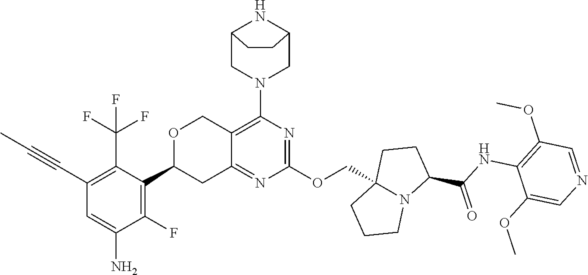 [SMILES: CC#Cc1cc(N)c(F)c([C@@H]2Cc3nc(OC[C@@]45CCCN4[C@H](C(=O)Nc4c(OC)cncc4OC)CC5)nc(N4CC5CCC(C4)N5)c3CO2)c1C(F)(F)F] | Example S77 |
| 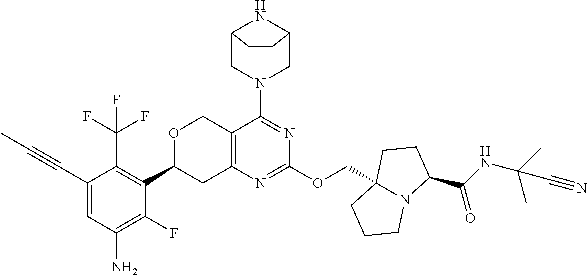 [SMILES: CC#Cc1cc(N)c(F)c([C@@H]2Cc3nc(OC[C@@]45CCCN4[C@H](C(=O)NC(C)(C)C#N)CC5)nc(N4CC5CCC(C4)N5)c3CO2)c1C(F)(F)F] | Example S78 |
| 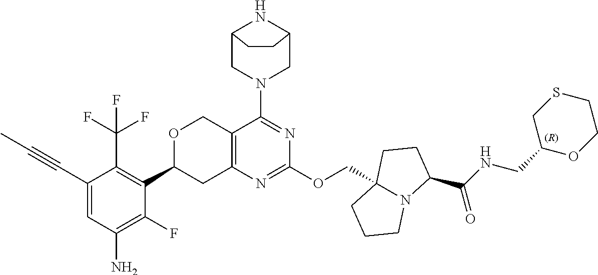 | Example S79 |
| 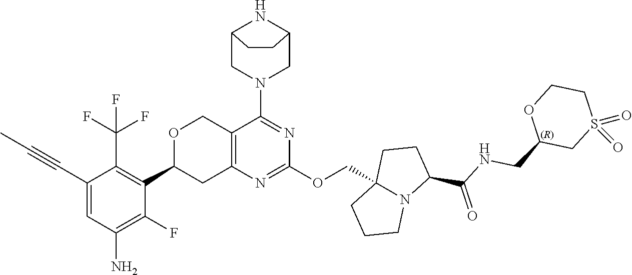 | Example S80 |
| 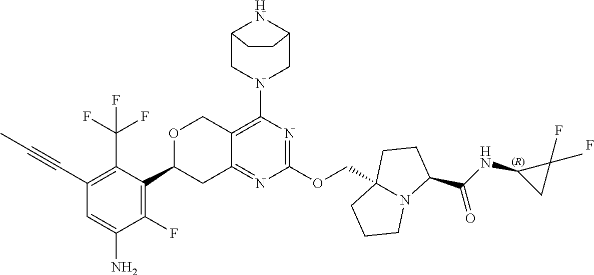 | Example S81 |
| 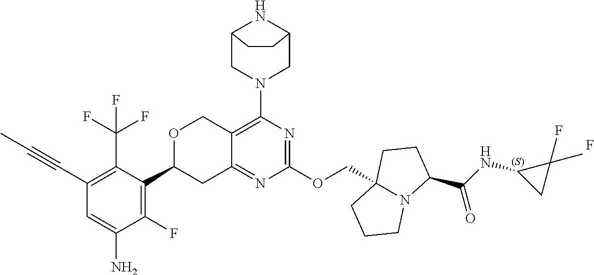 | Example S82 |
| 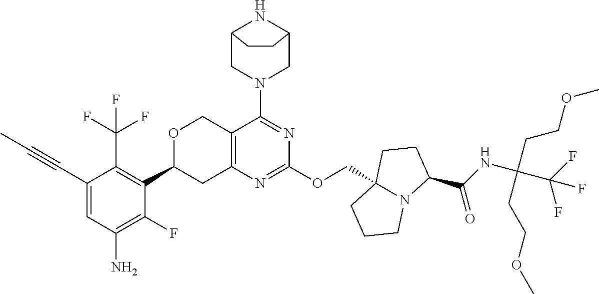 [SMILES: CC#Cc1cc(N)c(F)c([C@@H]2Cc3nc(OC[C@@]45CCCN4[C@H](C(=O)NC(CCOC)(CCOC)C(F)(F)F)CC5)nc(N4CC5CCC(C4)N5)c3CO2)c1C(F)(F)F] | Example S83 |
| 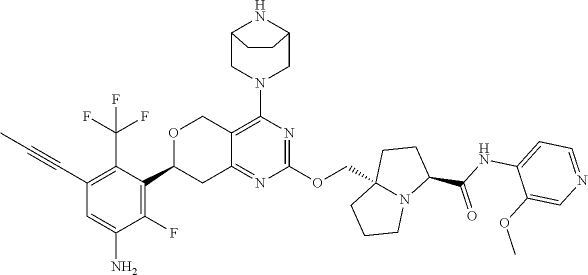 [SMILES: CC#Cc1cc(N)c(F)c([C@@H]2Cc3nc(OC[C@@]45CCCN4[C@H](C(=O)Nc4ccncc4OC)CC5)nc(N4CC5CCC(C4)N5)c3CO2)c1C(F)(F)F] | Example S84 |
| 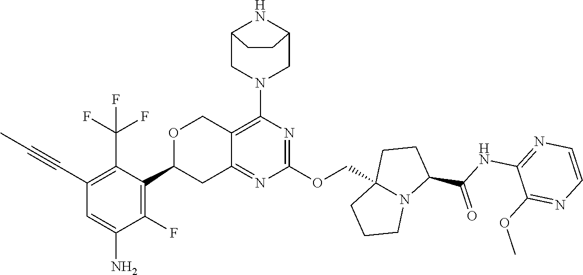 [SMILES: CC#Cc1cc(N)c(F)c([C@@H]2Cc3nc(OC[C@@]45CCCN4[C@H](C(=O)Nc4nccnc4OC)CC5)nc(N4CC5CCC(C4)N5)c3CO2)c1C(F)(F)F] | Example S85 |
| 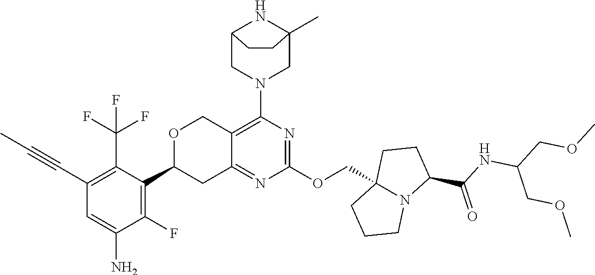 [SMILES: CC#Cc1cc(N)c(F)c([C@@H]2Cc3nc(OC[C@@]45CCCN4[C@H](C(=O)NC(COC)COC)CC5)nc(N4CC5CCC(C)(C4)N5)c3CO2)c1C(F)(F)F] | Example S86A/S86B |
| 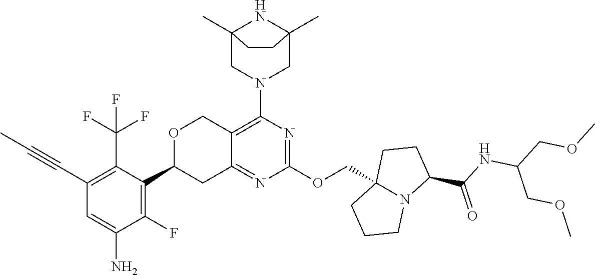 [SMILES: CC#Cc1cc(N)c(F)c([C@@H]2Cc3nc(OC[C@@]45CCCN4[C@H](C(=O)NC(COC)COC)CC5)nc(N4CC5(C)CCC(C)(C4)N5)c3CO2)c1C(F)(F)F] | Example S87 |
| 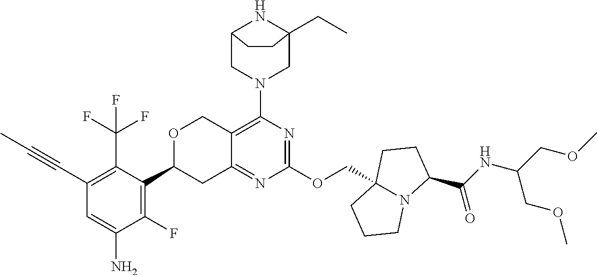 [SMILES: CC#Cc1cc(N)c(F)c([C@@H]2Cc3nc(OC[C@@]45CCCN4[C@H](C(=O)NC(COC)COC)CC5)nc(N4CC5CCC(CC)(C4)N5)c3CO2)c1C(F)(F)F] | Example S88A/S88B |
| 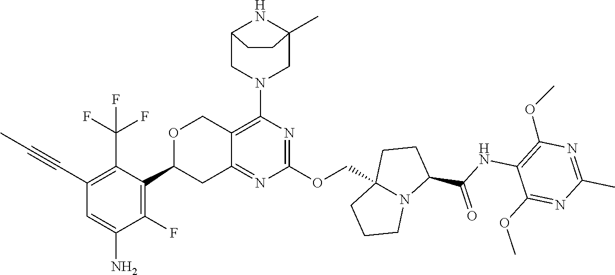 [SMILES: CC#Cc1cc(N)c(F)c([C@@H]2Cc3nc(OC[C@@]45CCCN4[C@H](C(=O)Nc4c(OC)nc(C)nc4OC)CC5)nc(N4CC5CCC(C)(C4)N5)c3CO2)c1C(F)(F)F] | Example S89A/S89B |
| 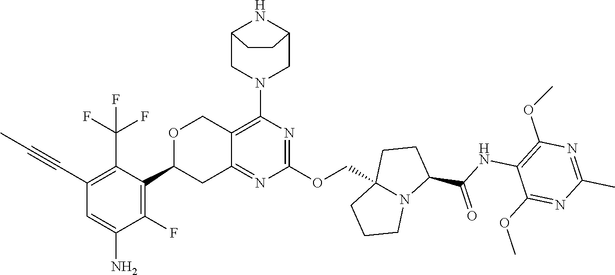 [SMILES: CC#Cc1cc(N)c(F)c([C@@H]2Cc3nc(OC[C@@]45CCCN4[C@H](C(=O)Nc4c(OC)nc(C)nc4OC)CC5)nc(N4CC5CCC(C4)N5)c3CO2)c1C(F)(F)F] | Example S90 |
| 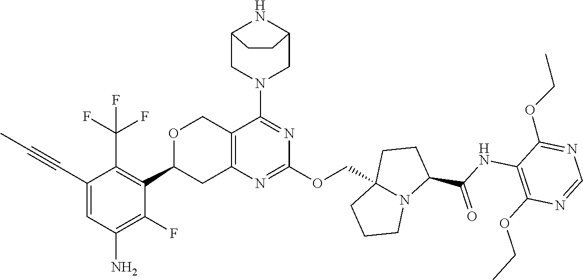 [SMILES: CC#Cc1cc(N)c(F)c([C@@H]2Cc3nc(OC[C@@]45CCCN4[C@H](C(=O)Nc4c(OCC)ncnc4OCC)CC5)nc(N4CC5CCC(C4)N5)c3CO2)c1C(F)(F)F] | Example S91 |
| 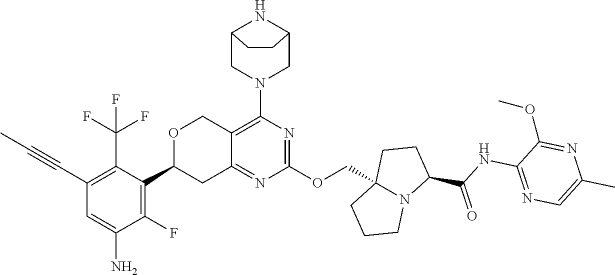 [SMILES: CC#Cc1cc(N)c(F)c([C@@H]2Cc3nc(OC[C@@]45CCCN4[C@H](C(=O)Nc4ncc(C)nc4OC)CC5)nc(N4CC5CCC(C4)N5)c3CO2)c1C(F)(F)F] | Example S92 |
| 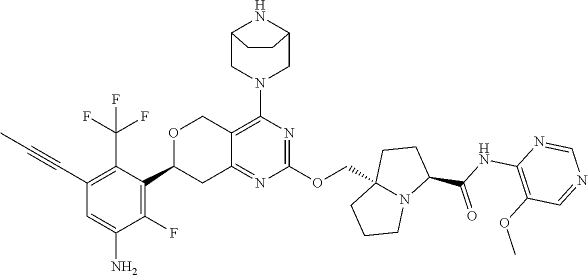 [SMILES: CC#Cc1cc(N)c(F)c([C@@H]2Cc3nc(OC[C@@]45CCCN4[C@H](C(=O)Nc4ncncc4OC)CC5)nc(N4CC5CCC(C4)N5)c3CO2)c1C(F)(F)F] | Example S93 |
| 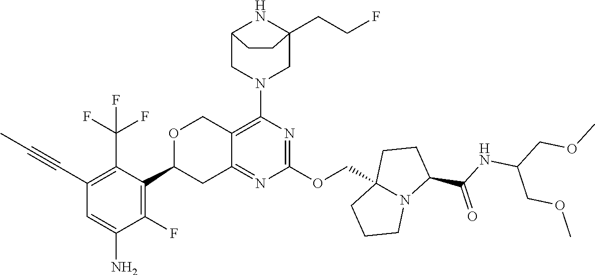 [SMILES: CC#Cc1cc(N)c(F)c([C@@H]2Cc3nc(OC[C@@]45CCCN4[C@H](C(=O)NC(COC)COC)CC5)nc(N4CC5CCC(CCF)(C4)N5)c3CO2)c1C(F)(F)F] | Example S94A/S94B |
| 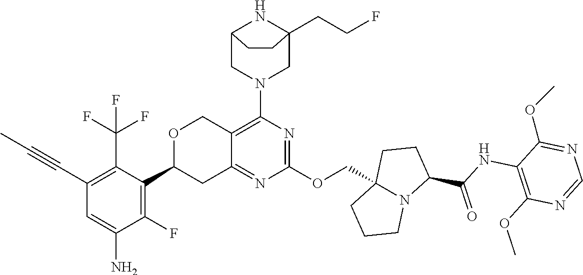 [SMILES: CC#Cc1cc(N)c(F)c([C@@H]2Cc3nc(OC[C@@]45CCCN4[C@H](C(=O)Nc4c(OC)ncnc4OC)CC5)nc(N4CC5CCC(CCF)(C4)N5)c3CO2)c1C(F)(F)F] | Example S95 |
| 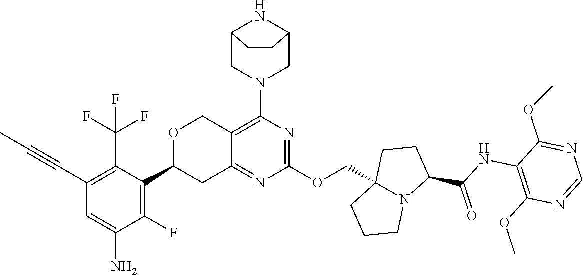 [SMILES: CC#Cc1cc(N)c(F)c([C@@H]2Cc3nc(OC[C@@]45CCCN4[C@H](C(=O)Nc4c(OC)ncnc4OC)CC5)nc(N4CC5CCC(C4)N5)c3CO2)c1C(F)(F)F] | Example S96 |

TABLE 5a

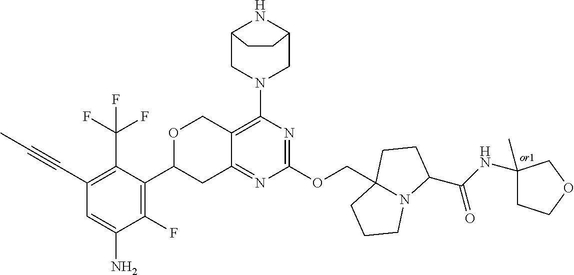 [SMILES: CC#Cc1cc(N)c(F)c(C2Cc3nc(OCC45CCCN4C(C(=O)NC4(C)CCOC4)CC5)nc(N4CC5CCC(C4)N5)c3CO2)c1C(F)(F)F]

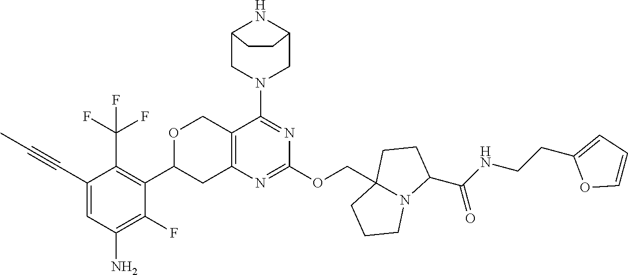 [SMILES: CC#Cc1cc(N)c(F)c(C2Cc3nc(OCC45CCCN4C(C(=O)NCCc4ccco4)CC5)nc(N4CC5CCC(C4)N5)c3CO2)c1C(F)(F)F]

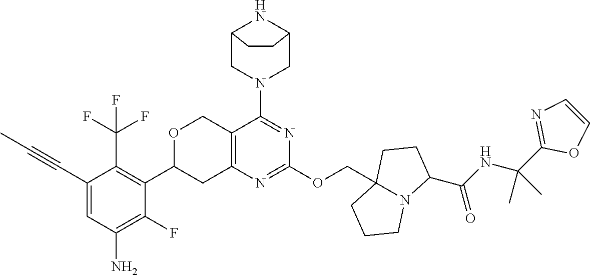 [SMILES: CC#Cc1cc(N)c(F)c(C2Cc3nc(OCC45CCCN4C(C(=O)NC(C)(C)c4ncco4)CC5)nc(N4CC5CCC(C4)N5)c3CO2)c1C(F)(F)F]

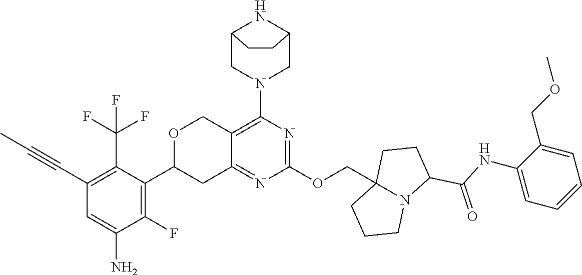 [SMILES: CC#Cc1cc(N)c(F)c(C2Cc3nc(OCC45CCCN4C(C(=O)Nc4ccccc4COC)CC5)nc(N4CC5CCC(C4)N5)c3CO2)c1C(F)(F)F]

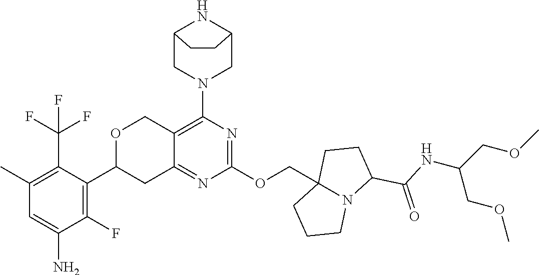

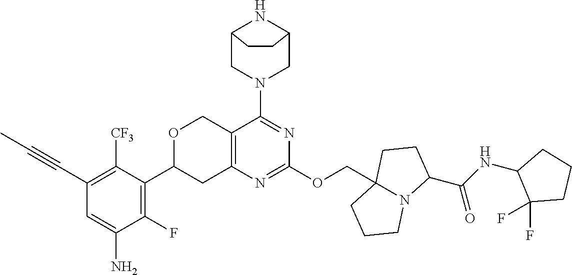 [SMILES: CC#Cc1cc(N)c(F)c(C2Cc3nc(OCC45CCCN4C(C(=O)NC4CCCC4(F)F)CC5)nc(N4CC5CCC(C4)N5)c3CO2)c1C(F)(F)F]

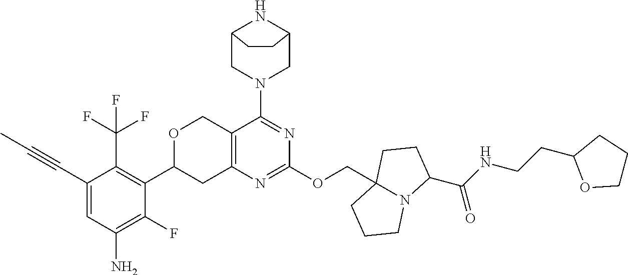 [SMILES: CC#Cc1cc(N)c(F)c(C2Cc3nc(OCC45CCCN4C(C(=O)NCCC4CCCO4)CC5)nc(N4CC5CCC(C4)N5)c3CO2)c1C(F)(F)F]

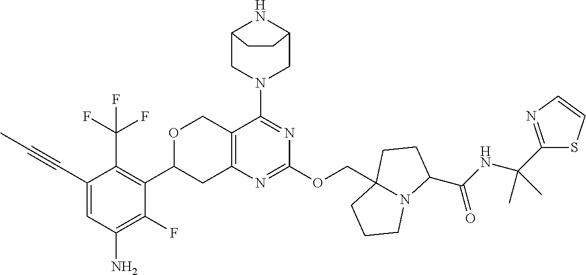 [SMILES: CC#Cc1cc(N)c(F)c(C2Cc3nc(OCC45CCCN4C(C(=O)NC(C)(C)c4nccs4)CC5)nc(N4CC5CCC(C4)N5)c3CO2)c1C(F)(F)F]

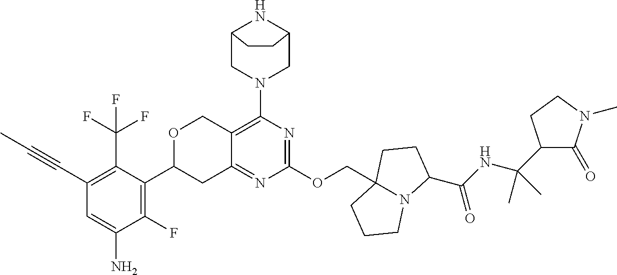 [SMILES: CC#Cc1cc(N)c(F)c(C2Cc3nc(OCC45CCCN4C(C(=O)NC(C)(C)C4CCN(C)C4=O)CC5)nc(N4CC5CCC(C4)N5)c3CO2)c1C(F)(F)F]

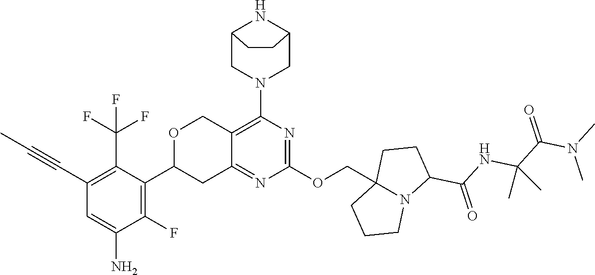 [SMILES: CC#Cc1cc(N)c(F)c(C2Cc3nc(OCC45CCCN4C(C(=O)NC(C)(C)C(=O)N(C)C)CC5)nc(N4CC5CCC(C4)N5)c3CO2)c1C(F)(F)F]

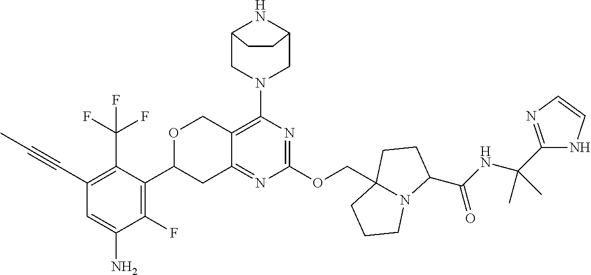 [SMILES: CC#Cc1cc(N)c(F)c(C2Cc3nc(OCC45CCCN4C(C(=O)NC(C)(C)c4ncc[nH]4)CC5)nc(N4CC5CCC(C4)N5)c3CO2)c1C(F)(F)F]

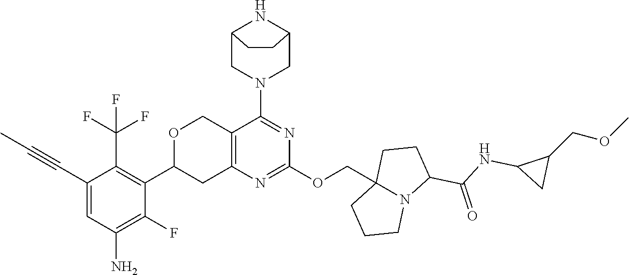 [SMILES: CC#Cc1cc(N)c(F)c(C2Cc3nc(OCC45CCCN4C(C(=O)NC4CC4COC)CC5)nc(N4CC5CCC(C4)N5)c3CO2)c1C(F)(F)F]

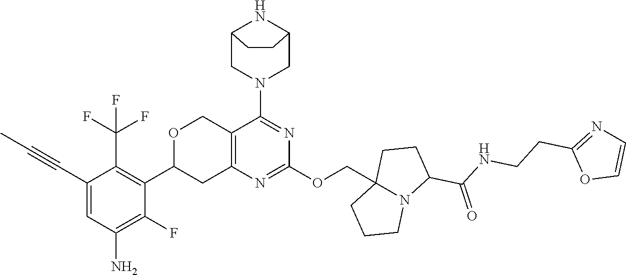 [SMILES: CC#Cc1cc(N)c(F)c(C2Cc3nc(OCC45CCCN4C(C(=O)NCCc4ncco4)CC5)nc(N4CC5CCC(C4)N5)c3CO2)c1C(F)(F)F]

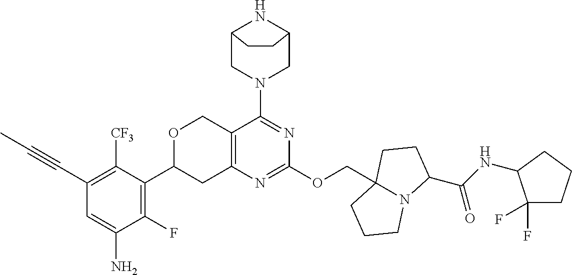 [SMILES: CC#Cc1cc(N)c(F)c(C2Cc3nc(OCC45CCCN4C(C(=O)NC4CCCC4(F)F)CC5)nc(N4CC5CCC(C4)N5)c3CO2)c1C(F)(F)F]

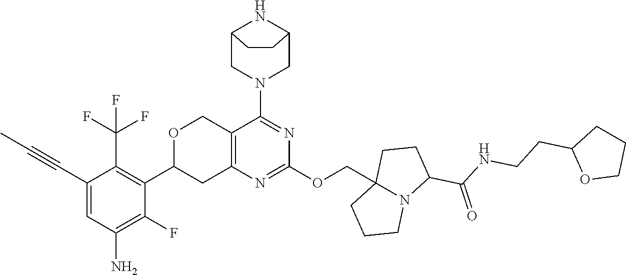 [SMILES: CC#Cc1cc(N)c(F)c(C2Cc3nc(OCC45CCCN4C(C(=O)NCCC4CCCO4)CC5)nc(N4CC5CCC(C4)N5)c3CO2)c1C(F)(F)F]

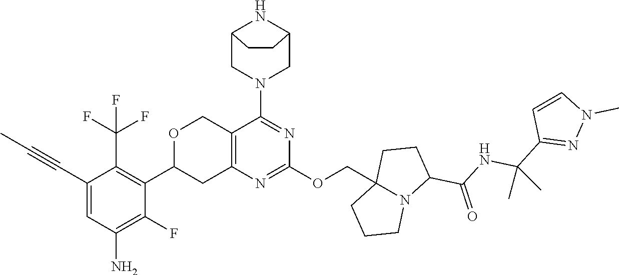 [SMILES: CC#Cc1cc(N)c(F)c(C2Cc3nc(OCC45CCCN4C(C(=O)NC(C)(C)c4ccn(C)n4)CC5)nc(N4CC5CCC(C4)N5)c3CO2)c1C(F)(F)F]

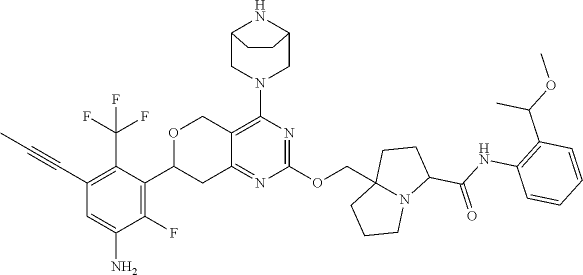 [SMILES: CC#Cc1cc(N)c(F)c(C2Cc3nc(OCC45CCCN4C(C(=O)Nc4ccccc4C(C)OC)CC5)nc(N4CC5CCC(C4)N5)c3CO2)c1C(F)(F)F]

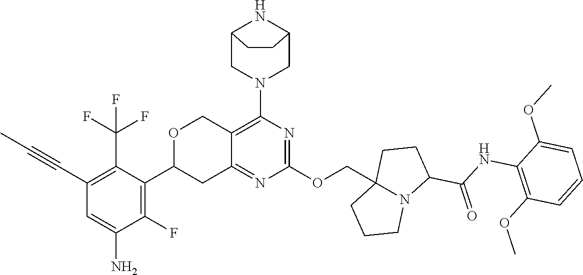 [SMILES: CC#Cc1cc(N)c(F)c(C2Cc3nc(OCC45CCCN4C(C(=O)Nc4c(OC)cccc4OC)CC5)nc(N4CC5CCC(C4)N5)c3CO2)c1C(F)(F)F]

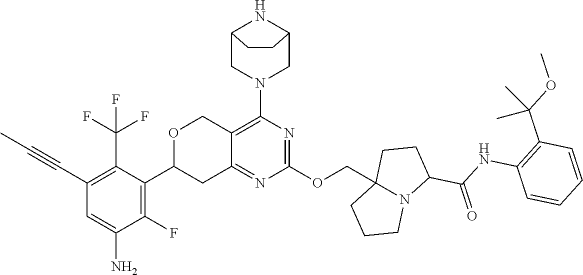 [SMILES: CC#Cc1cc(N)c(F)c(C2Cc3nc(OCC45CCCN4C(C(=O)Nc4ccccc4C(C)(C)OC)CC5)nc(N4CC5CCC(C4)N5)c3CO2)c1C(F)(F)F]

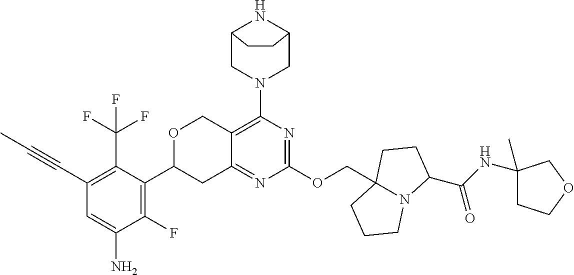 [SMILES: CC#Cc1cc(N)c(F)c(C2Cc3nc(OCC45CCCN4C(C(=O)NC4(C)CCOC4)CC5)nc(N4CC5CCC(C4)N5)c3CO2)c1C(F)(F)F]

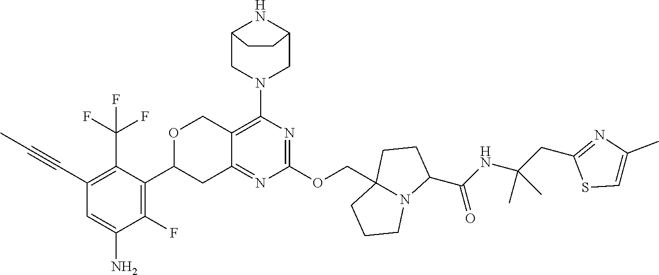 [SMILES: CC#Cc1cc(N)c(F)c(C2Cc3nc(OCC45CCCN4C(C(=O)NC(C)(C)Cc4nc(C)cs4)CC5)nc(N4CC5CCC(C4)N5)c3CO2)c1C(F)(F)F]

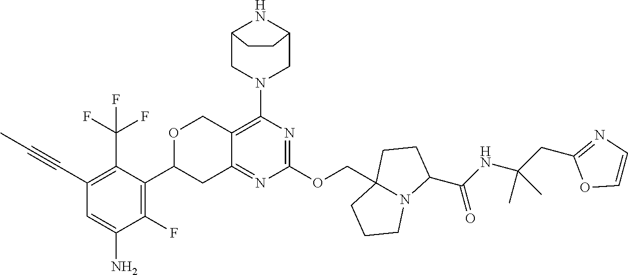 [SMILES: CC#Cc1cc(N)c(F)c(C2Cc3nc(OCC45CCCN4C(C(=O)NC(C)(C)Cc4ncco4)CC5)nc(N4CC5CCC(C4)N5)c3CO2)c1C(F)(F)F]

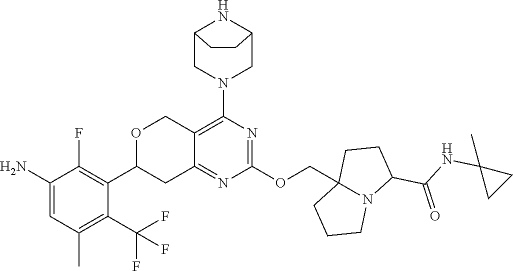 [SMILES: Cc1cc(N)c(F)c(C2Cc3nc(OCC45CCCN4C(C(=O)NC4(C)CC4)CC5)nc(N4CC5CCC(C4)N5)c3CO2)c1C(F)(F)F]

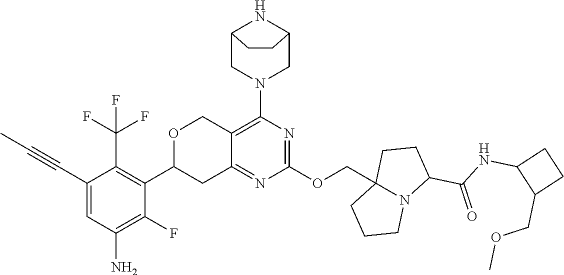 [SMILES: CC#Cc1cc(N)c(F)c(C2Cc3nc(OCC45CCCN4C(C(=O)NC4CCC4COC)CC5)nc(N4CC5CCC(C4)N5)c3CO2)c1C(F)(F)F]

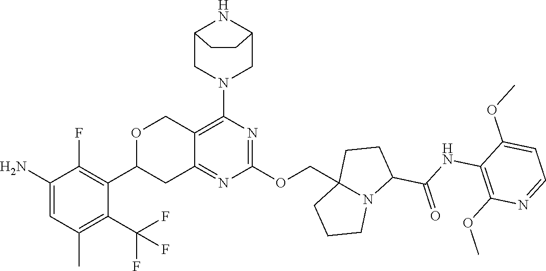 [SMILES: COc1ccnc(OC)c1NC(=O)C1CCC2(COc3nc4c(c(N5CC6CCC(C5)N6)n3)COC(c3c(F)c(N)cc(C)c3C(F)(F)F)C4)CCCN12]

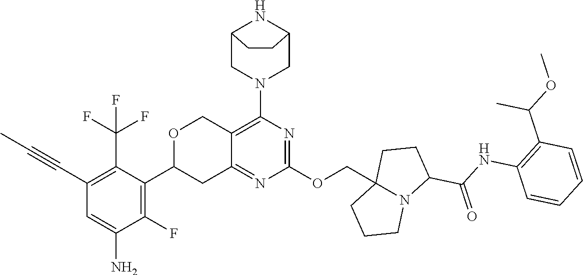 [SMILES: CC#Cc1cc(N)c(F)c(C2Cc3nc(OCC45CCCN4C(C(=O)Nc4ccccc4C(C)OC)CC5)nc(N4CC5CCC(C4)N5)c3CO2)c1C(F)(F)F]

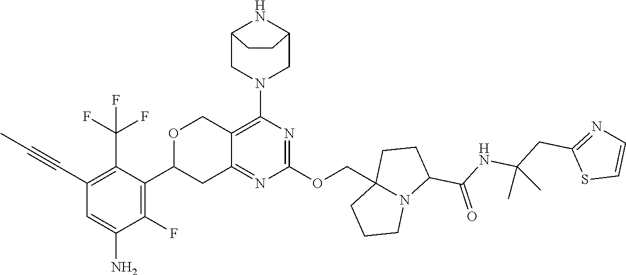 [SMILES: CC#Cc1cc(N)c(F)c(C2Cc3nc(OCC45CCCN4C(C(=O)NC(C)(C)Cc4nccs4)CC5)nc(N4CC5CCC(C4)N5)c3CO2)c1C(F)(F)F]

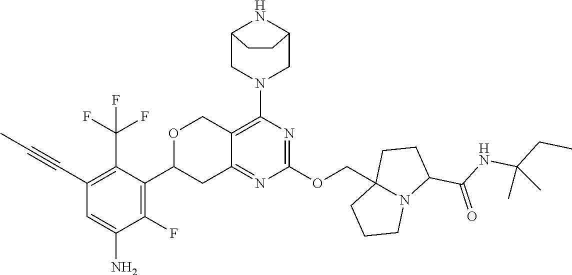 [SMILES: CC#Cc1cc(N)c(F)c(C2Cc3nc(OCC45CCCN4C(C(=O)NC(C)(C)CC)CC5)nc(N4CC5CCC(C4)N5)c3CO2)c1C(F)(F)F]

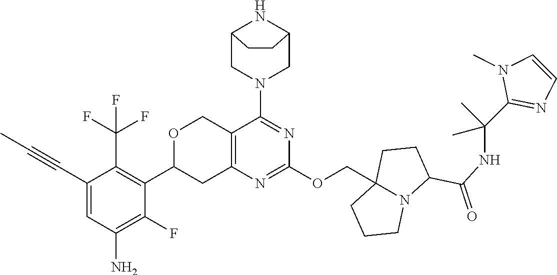 [SMILES: CC#Cc1cc(N)c(F)c(C2Cc3nc(OCC45CCCN4C(C(=O)NC(C)(C)c4nccn4C)CC5)nc(N4CC5CCC(C4)N5)c3CO2)c1C(F)(F)F]

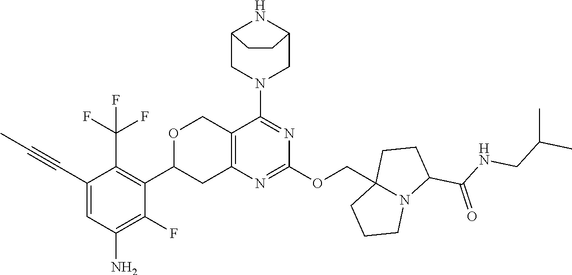 [SMILES: CC#Cc1cc(N)c(F)c(C2Cc3nc(OCC45CCCN4C(C(=O)NCC(C)C)CC5)nc(N4CC5CCC(C4)N5)c3CO2)c1C(F)(F)F]

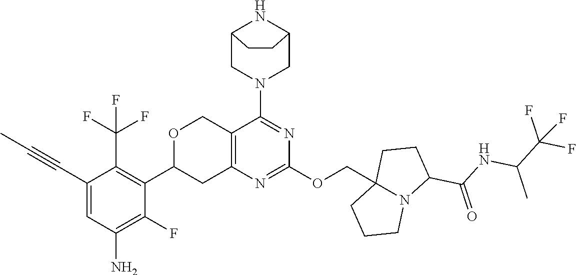 [SMILES: CC#Cc1cc(N)c(F)c(C2Cc3nc(OCC45CCCN4C(C(=O)NC(C)C(F)(F)F)CC5)nc(N4CC5CCC(C4)N5)c3CO2)c1C(F)(F)F]

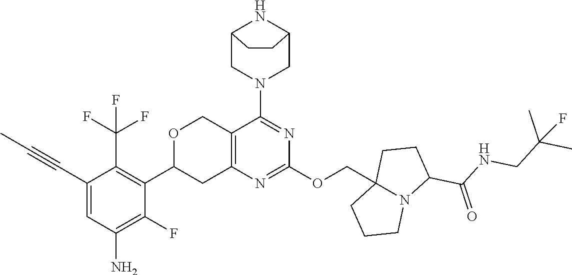 [SMILES: CC#Cc1cc(N)c(F)c(C2Cc3nc(OCC45CCCN4C(C(=O)NCC(C)(C)F)CC5)nc(N4CC5CCC(C4)N5)c3CO2)c1C(F)(F)F]

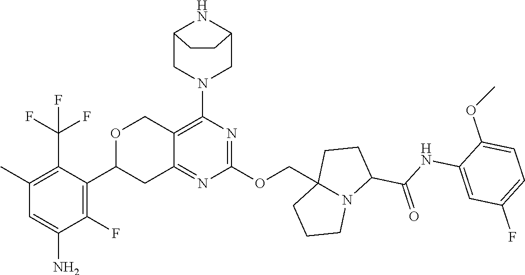 [SMILES: COc1ccc(F)cc1NC(=O)C1CCC2(COc3nc4c(c(N5CC6CCC(C5)N6)n3)COC(c3c(F)c(N)cc(C)c3C(F)(F)F)C4)CCCN12]

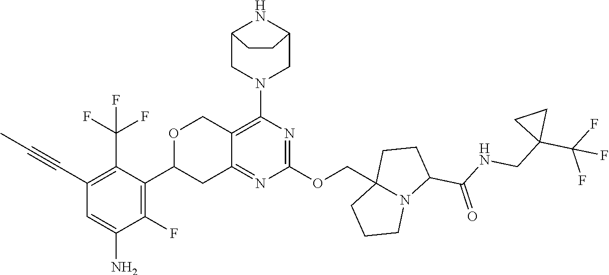 [SMILES: CC#Cc1cc(N)c(F)c(C2Cc3nc(OCC45CCCN4C(C(=O)NCC4(C(F)(F)F)CC4)CC5)nc(N4CC5CCC(C4)N5)c3CO2)c1C(F)(F)F]

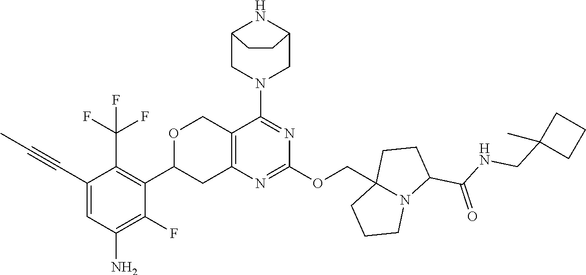 [SMILES: CC#Cc1cc(N)c(F)c(C2Cc3nc(OCC45CCCN4C(C(=O)NCC4(C)CCC4)CC5)nc(N4CC5CCC(C4)N5)c3CO2)c1C(F)(F)F]

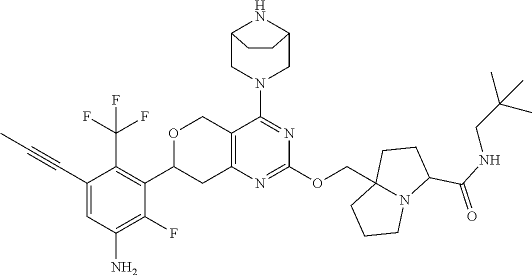 [SMILES: CC#Cc1cc(N)c(F)c(C2Cc3nc(OCC45CCCN4C(C(=O)NCC(C)(C)C)CC5)nc(N4CC5CCC(C4)N5)c3CO2)c1C(F)(F)F]

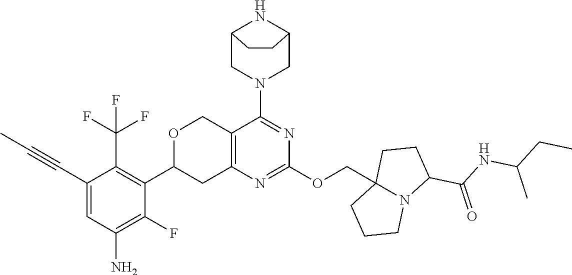 [SMILES: CC#Cc1cc(N)c(F)c(C2Cc3nc(OCC45CCCN4C(C(=O)NC(C)CC)CC5)nc(N4CC5CCC(C4)N5)c3CO2)c1C(F)(F)F]

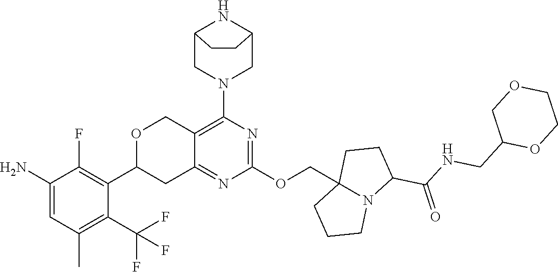 [SMILES: Cc1cc(N)c(F)c(C2Cc3nc(OCC45CCCN4C(C(=O)NCC4COCCO4)CC5)nc(N4CC5CCC(C4)N5)c3CO2)c1C(F)(F)F]

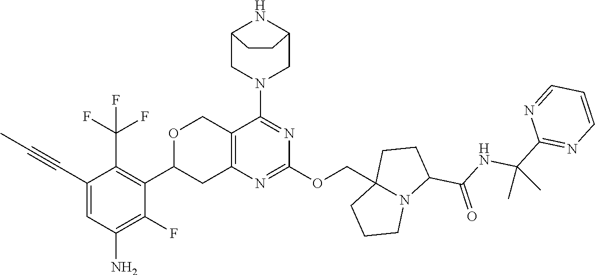 [SMILES: CC#Cc1cc(N)c(F)c(C2Cc3nc(OCC45CCCN4C(C(=O)NC(C)(C)c4ncccn4)CC5)nc(N4CC5CCC(C4)N5)c3CO2)c1C(F)(F)F]

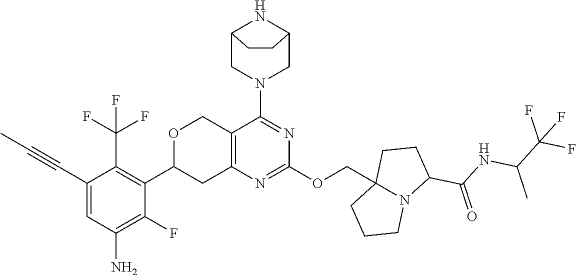 [SMILES: CC#Cc1cc(N)c(F)c(C2Cc3nc(OCC45CCCN4C(C(=O)NC(C)C(F)(F)F)CC5)nc(N4CC5CCC(C4)N5)c3CO2)c1C(F)(F)F]

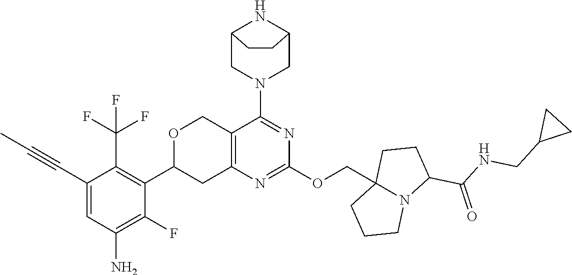 [SMILES: CC#Cc1cc(N)c(F)c(C2Cc3nc(OCC45CCCN4C(C(=O)NCC4CC4)CC5)nc(N4CC5CCC(C4)N5)c3CO2)c1C(F)(F)F]

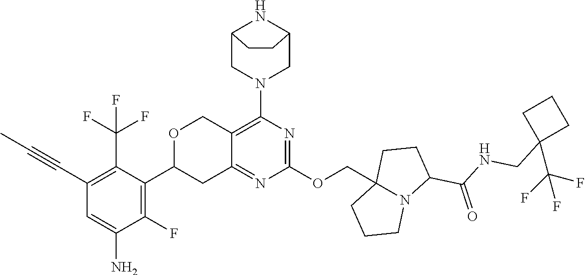 [SMILES: CC#Cc1cc(N)c(F)c(C2Cc3nc(OCC45CCCN4C(C(=O)NCC4(C(F)(F)F)CCC4)CC5)nc(N4CC5CCC(C4)N5)c3CO2)c1C(F)(F)F]

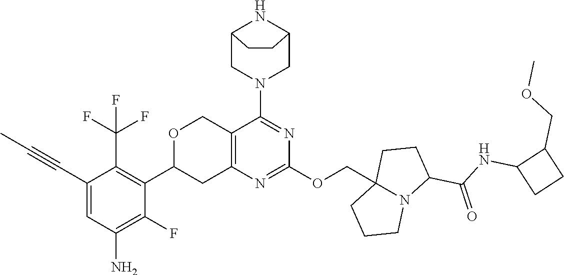 [SMILES: CC#Cc1cc(N)c(F)c(C2Cc3nc(OCC45CCCN4C(C(=O)NC4CCC4COC)CC5)nc(N4CC5CCC(C4)N5)c3CO2)c1C(F)(F)F]

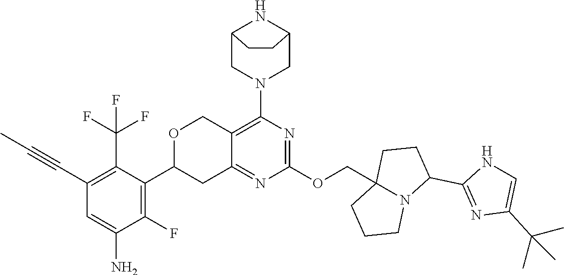 [SMILES: CC#Cc1cc(N)c(F)c(C2Cc3nc(OCC45CCCN4C(c4nc(C(C)(C)C)c[nH]4)CC5)nc(N4CC5CCC(C4)N5)c3CO2)c1C(F)(F)F]

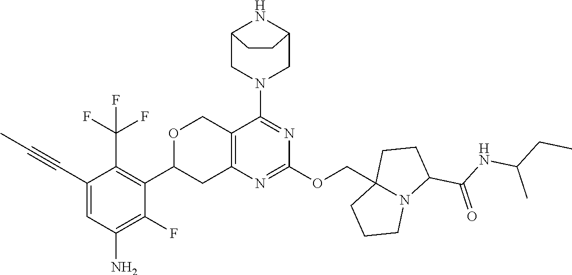 [SMILES: CC#Cc1cc(N)c(F)c(C2Cc3nc(OCC45CCCN4C(C(=O)NC(C)CC)CC5)nc(N4CC5CCC(C4)N5)c3CO2)c1C(F)(F)F]

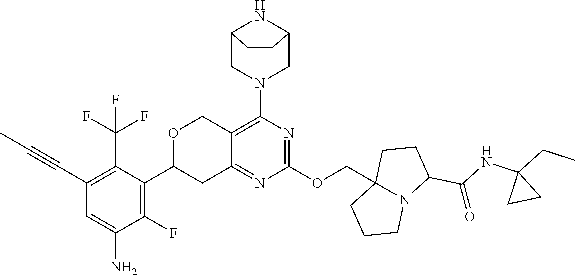 [SMILES: CC#Cc1cc(N)c(F)c(C2Cc3nc(OCC45CCCN4C(C(=O)NC4(CC)CC4)CC5)nc(N4CC5CCC(C4)N5)c3CO2)c1C(F)(F)F]

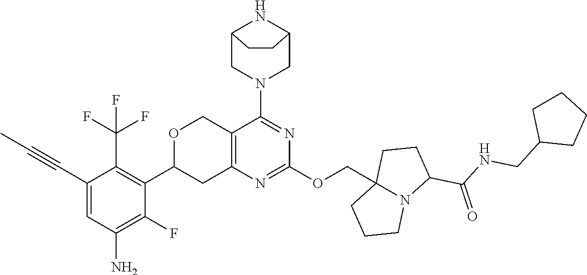 [SMILES: CC#Cc1cc(N)c(F)c(C2Cc3nc(OCC45CCCN4C(C(=O)NCC4CCCC4)CC5)nc(N4CC5CCC(C4)N5)c3CO2)c1C(F)(F)F]

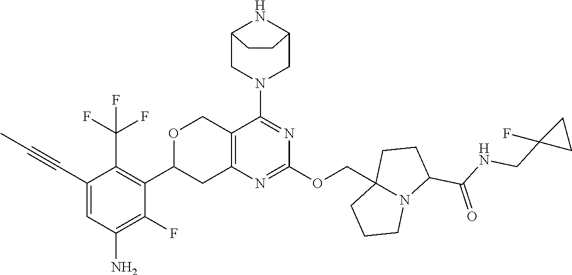 [SMILES: CC#Cc1cc(N)c(F)c(C2Cc3nc(OCC45CCCN4C(C(=O)NCC4(F)CC4)CC5)nc(N4CC5CCC(C4)N5)c3CO2)c1C(F)(F)F]

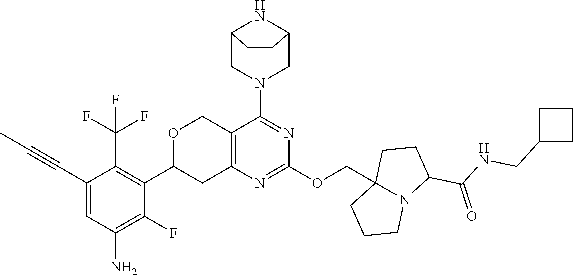 [SMILES: CC#Cc1cc(N)c(F)c(C2Cc3nc(OCC45CCCN4C(C(=O)NCC4CCC4)CC5)nc(N4CC5CCC(C4)N5)c3CO2)c1C(F)(F)F]

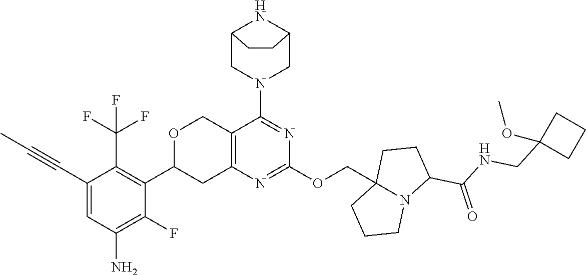 [SMILES: CC#Cc1cc(N)c(F)c(C2Cc3nc(OCC45CCCN4C(C(=O)NCC4(OC)CCC4)CC5)nc(N4CC5CCC(C4)N5)c3CO2)c1C(F)(F)F]

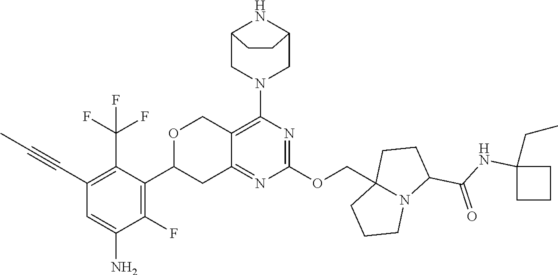 [SMILES: CC#Cc1cc(N)c(F)c(C2Cc3nc(OCC45CCCN4C(C(=O)NC4(CC)CCC4)CC5)nc(N4CC5CCC(C4)N5)c3CO2)c1C(F)(F)F]

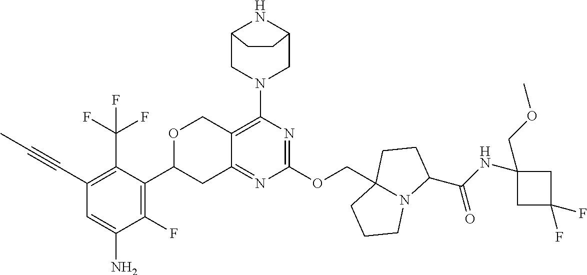 [SMILES: CC#Cc1cc(N)c(F)c(C2Cc3nc(OCC45CCCN4C(C(=O)NC4(COC)CC(F)(F)C4)CC5)nc(N4CC5CCC(C4)N5)c3CO2)c1C(F)(F)F]

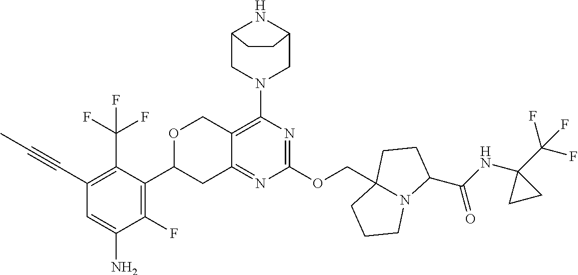 [SMILES: CC#Cc1cc(N)c(F)c(C2Cc3nc(OCC45CCCN4C(C(=O)NC4(C(F)(F)F)CC4)CC5)nc(N4CC5CCC(C4)N5)c3CO2)c1C(F)(F)F]

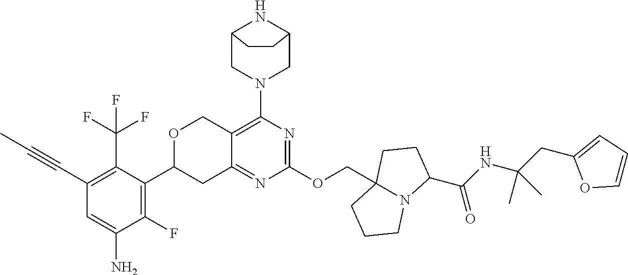 [SMILES: CC#Cc1cc(N)c(F)c(C2Cc3nc(OCC45CCCN4C(C(=O)NC(C)(C)Cc4ccco4)CC5)nc(N4CC5CCC(C4)N5)c3CO2)c1C(F)(F)F]

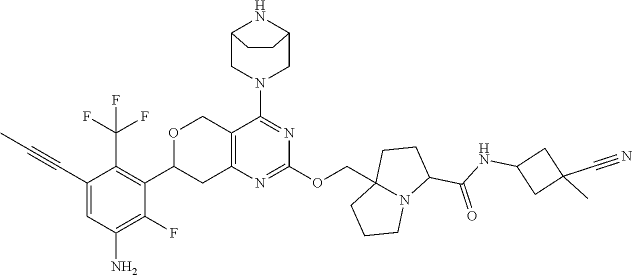 [SMILES: CC#Cc1cc(N)c(F)c(C2Cc3nc(OCC45CCCN4C(C(=O)NC4CC(C)(C#N)C4)CC5)nc(N4CC5CCC(C4)N5)c3CO2)c1C(F)(F)F]

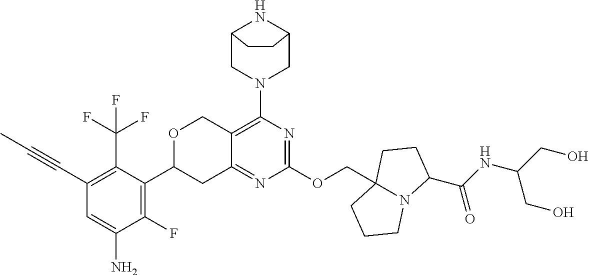 [SMILES: CC#Cc1cc(N)c(F)c(C2Cc3nc(OCC45CCCN4C(C(=O)NC(CO)CO)CC5)nc(N4CC5CCC(C4)N5)c3CO2)c1C(F)(F)F]

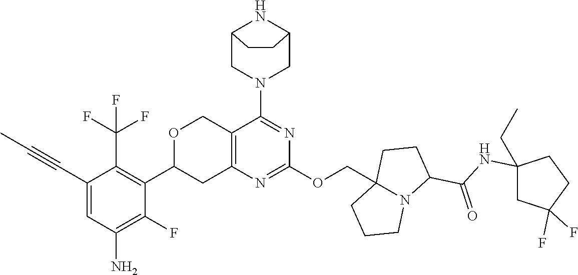 [SMILES: CC#Cc1cc(N)c(F)c(C2Cc3nc(OCC45CCCN4C(C(=O)NC4(CC)CCC(F)(F)C4)CC5)nc(N4CC5CCC(C4)N5)c3CO2)c1C(F)(F)F]

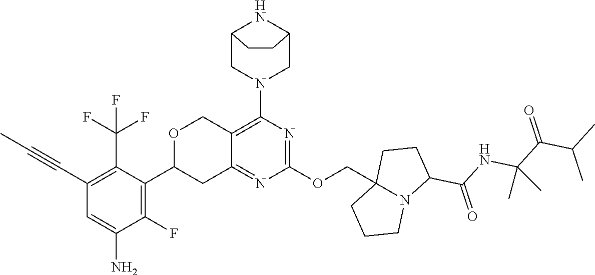 [SMILES: CC#Cc1cc(N)c(F)c(C2Cc3nc(OCC45CCCN4C(C(=O)NC(C)(C)C(=O)C(C)C)CC5)nc(N4CC5CCC(C4)N5)c3CO2)c1C(F)(F)F]

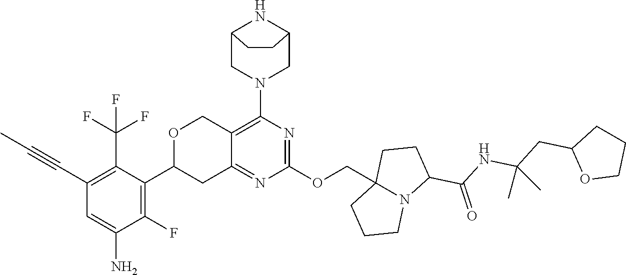 [SMILES: CC#Cc1cc(N)c(F)c(C2Cc3nc(OCC45CCCN4C(C(=O)NC(C)(C)CC4CCCO4)CC5)nc(N4CC5CCC(C4)N5)c3CO2)c1C(F)(F)F]

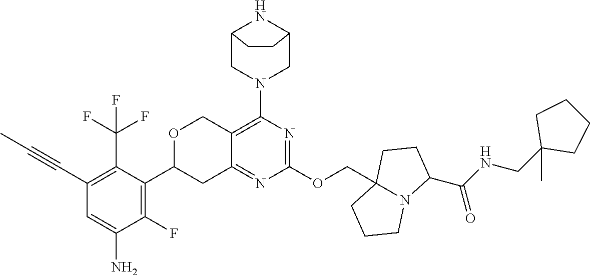 [SMILES: CC#Cc1cc(N)c(F)c(C2Cc3nc(OCC45CCCN4C(C(=O)NCC4(C)CCCC4)CC5)nc(N4CC5CCC(C4)N5)c3CO2)c1C(F)(F)F]

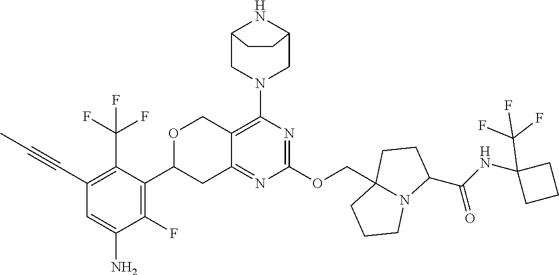 [SMILES: CC#Cc1cc(N)c(F)c(C2Cc3nc(OCC45CCCN4C(C(=O)NC4(C(F)(F)F)CCC4)CC5)nc(N4CC5CCC(C4)N5)c3CO2)c1C(F)(F)F]

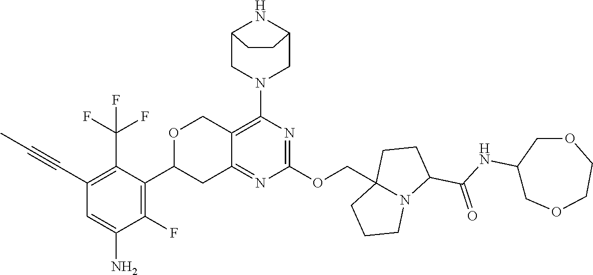 [SMILES: CC#Cc1cc(N)c(F)c(C2Cc3nc(OCC45CCCN4C(C(=O)NC4COCCOC4)CC5)nc(N4CC5CCC(C4)N5)c3CO2)c1C(F)(F)F]

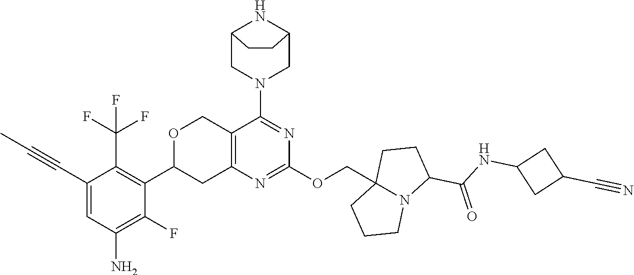 [SMILES: CC#Cc1cc(N)c(F)c(C2Cc3nc(OCC45CCCN4C(C(=O)NC4CC(C#N)C4)CC5)nc(N4CC5CCC(C4)N5)c3CO2)c1C(F)(F)F]

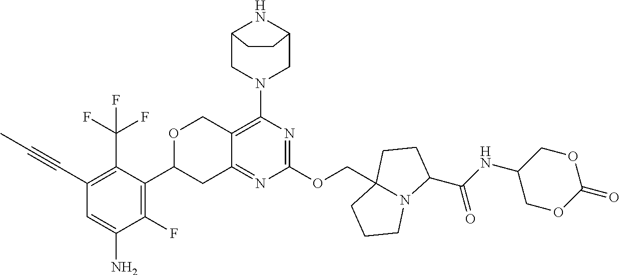 [SMILES: CC#Cc1cc(N)c(F)c(C2Cc3nc(OCC45CCCN4C(C(=O)NC4COC(=O)OC4)CC5)nc(N4CC5CCC(C4)N5)c3CO2)c1C(F)(F)F]

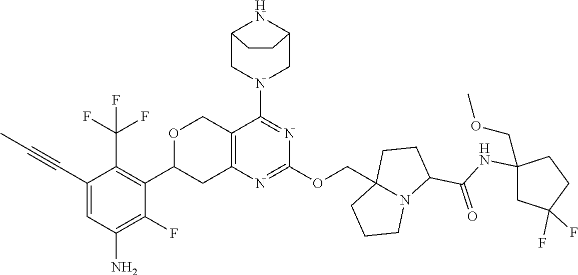 [SMILES: CC#Cc1cc(N)c(F)c(C2Cc3nc(OCC45CCCN4C(C(=O)NC4(COC)CCC(F)(F)C4)CC5)nc(N4CC5CCC(C4)N5)c3CO2)c1C(F)(F)F]

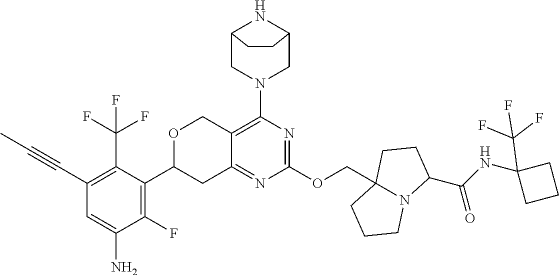 [SMILES: CC#Cc1cc(N)c(F)c(C2Cc3nc(OCC45CCCN4C(C(=O)NC4(C(F)(F)F)CCC4)CC5)nc(N4CC5CCC(C4)N5)c3CO2)c1C(F)(F)F]

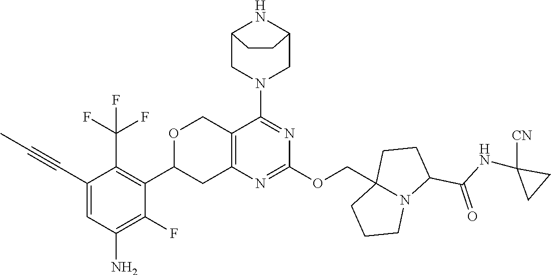 [SMILES: CC#Cc1cc(N)c(F)c(C2Cc3nc(OCC45CCCN4C(C(=O)NC4(C#N)CC4)CC5)nc(N4CC5CCC(C4)N5)c3CO2)c1C(F)(F)F]

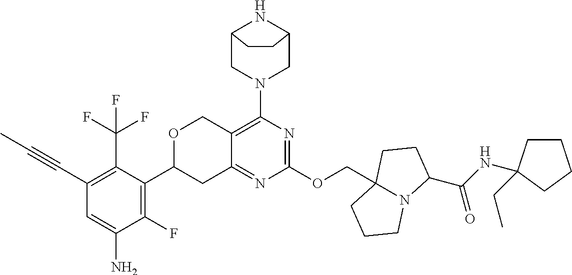 [SMILES: CC#Cc1cc(N)c(F)c(C2Cc3nc(OCC45CCCN4C(C(=O)NC4(CC)CCCC4)CC5)nc(N4CC5CCC(C4)N5)c3CO2)c1C(F)(F)F]

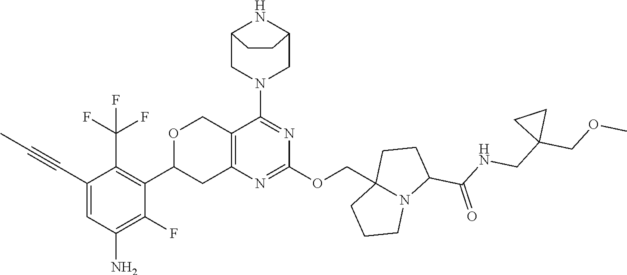 [SMILES: CC#Cc1cc(N)c(F)c(C2Cc3nc(OCC45CCCN4C(C(=O)NCC4(COC)CC4)CC5)nc(N4CC5CCC(C4)N5)c3CO2)c1C(F)(F)F]

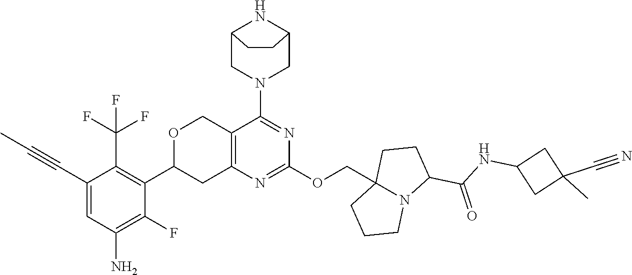 [SMILES: CC#Cc1cc(N)c(F)c(C2Cc3nc(OCC45CCCN4C(C(=O)NC4CC(C)(C#N)C4)CC5)nc(N4CC5CCC(C4)N5)c3CO2)c1C(F)(F)F]

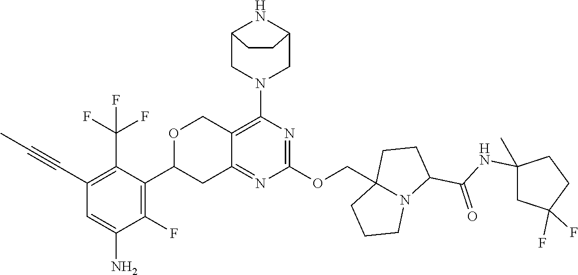 [SMILES: CC#Cc1cc(N)c(F)c(C2Cc3nc(OCC45CCCN4C(C(=O)NC4(C)CCC(F)(F)C4)CC5)nc(N4CC5CCC(C4)N5)c3CO2)c1C(F)(F)F]

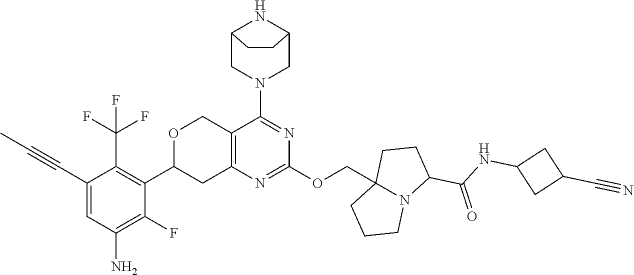 [SMILES: CC#Cc1cc(N)c(F)c(C2Cc3nc(OCC45CCCN4C(C(=O)NC4CC(C#N)C4)CC5)nc(N4CC5CCC(C4)N5)c3CO2)c1C(F)(F)F]

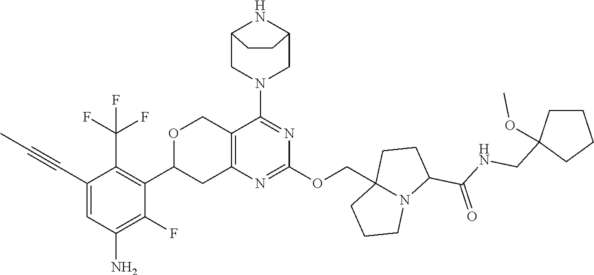 [SMILES: CC#Cc1cc(N)c(F)c(C2Cc3nc(OCC45CCCN4C(C(=O)NCC4(OC)CCCC4)CC5)nc(N4CC5CCC(C4)N5)c3CO2)c1C(F)(F)F]

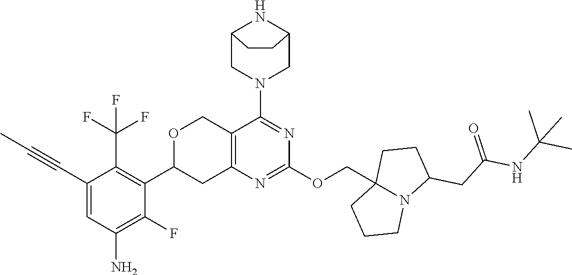 [SMILES: CC#Cc1cc(N)c(F)c(C2Cc3nc(OCC45CCCN4C(CC(=O)NC(C)(C)C)CC5)nc(N4CC5CCC(C4)N5)c3CO2)c1C(F)(F)F]

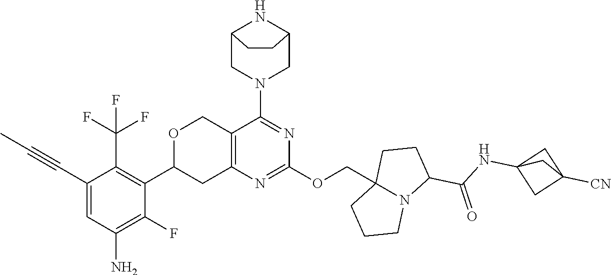 [SMILES: CC#Cc1cc(N)c(F)c(C2Cc3nc(OCC45CCCN4C(C(=O)NC46CC(C#N)(C4)C6)CC5)nc(N4CC5CCC(C4)N5)c3CO2)c1C(F)(F)F]

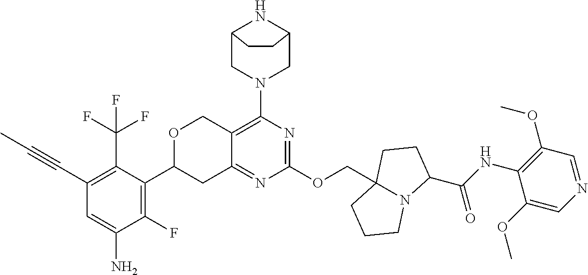 [SMILES: CC#Cc1cc(N)c(F)c(C2Cc3nc(OCC45CCCN4C(C(=O)Nc4c(OC)cncc4OC)CC5)nc(N4CC5CCC(C4)N5)c3CO2)c1C(F)(F)F]

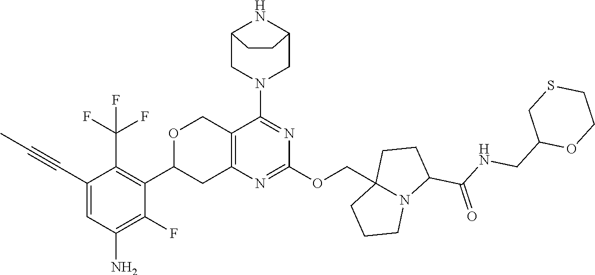 [SMILES: CC#Cc1cc(N)c(F)c(C2Cc3nc(OCC45CCCN4C(C(=O)NCC4CSCCO4)CC5)nc(N4CC5CCC(C4)N5)c3CO2)c1C(F)(F)F]

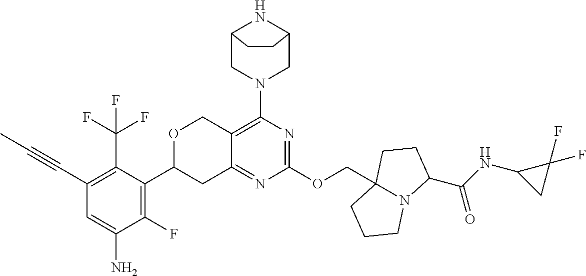 [SMILES: CC#Cc1cc(N)c(F)c(C2Cc3nc(OCC45CCCN4C(C(=O)NC4CC4(F)F)CC5)nc(N4CC5CCC(C4)N5)c3CO2)c1C(F)(F)F]

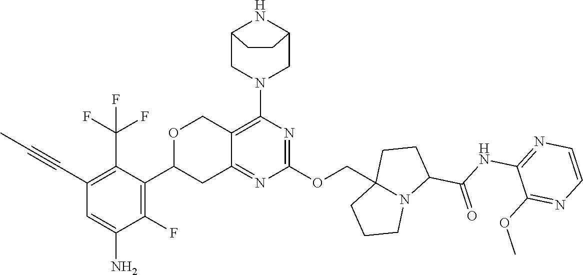 [SMILES: CC#Cc1cc(N)c(F)c(C2Cc3nc(OCC45CCCN4C(C(=O)Nc4nccnc4OC)CC5)nc(N4CC5CCC(C4)N5)c3CO2)c1C(F)(F)F]

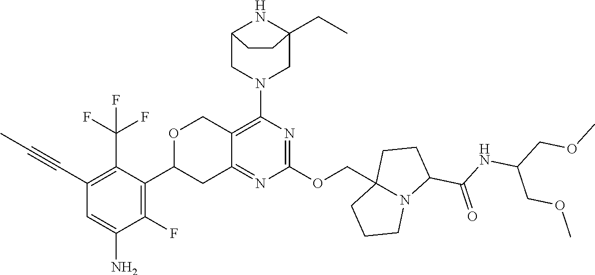 [SMILES: CC#Cc1cc(N)c(F)c(C2Cc3nc(OCC45CCCN4C(C(=O)NC(COC)COC)CC5)nc(N4CC5CCC(CC)(C4)N5)c3CO2)c1C(F)(F)F]

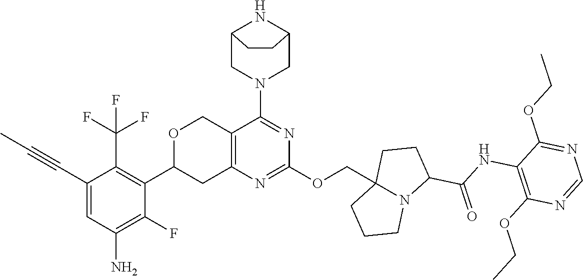 [SMILES: CC#Cc1cc(N)c(F)c(C2Cc3nc(OCC45CCCN4C(C(=O)Nc4c(OCC)ncnc4OCC)CC5)nc(N4CC5CCC(C4)N5)c3CO2)c1C(F)(F)F]

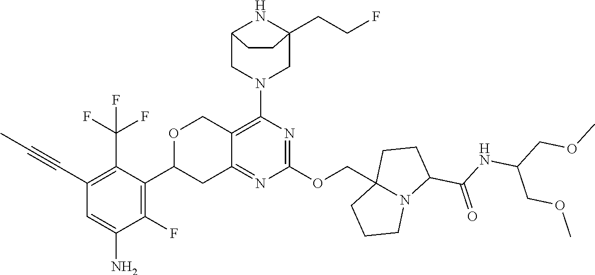 [SMILES: CC#Cc1cc(N)c(F)c(C2Cc3nc(OCC45CCCN4C(C(=O)NC(COC)COC)CC5)nc(N4CC5CCC(CCF)(C4)N5)c3CO2)c1C(F)(F)F]

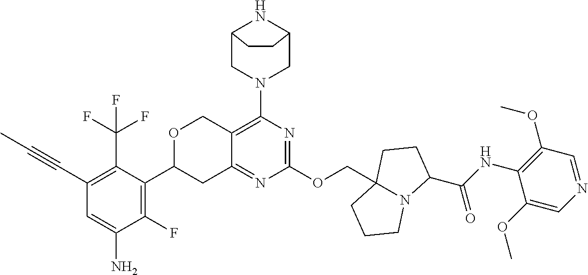 [SMILES: CC#Cc1cc(N)c(F)c(C2Cc3nc(OCC45CCCN4C(C(=O)Nc4c(OC)cncc4OC)CC5)nc(N4CC5CCC(C4)N5)c3CO2)c1C(F)(F)F]

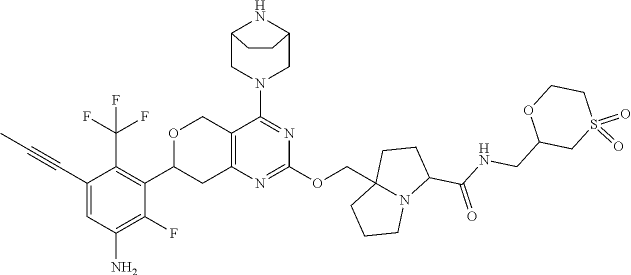 [SMILES: CC#Cc1cc(N)c(F)c(C2Cc3nc(OCC45CCCN4C(C(=O)NCC4CS(=O)(=O)CCO4)CC5)nc(N4CC5CCC(C4)N5)c3CO2)c1C(F)(F)F]

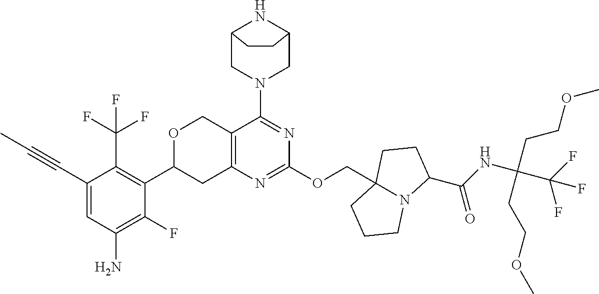 [SMILES: CC#Cc1cc(N)c(F)c(C2Cc3nc(OCC45CCCN4C(C(=O)NC(CCOC)(CCOC)C(F)(F)F)CC5)nc(N4CC5CCC(C4)N5)c3CO2)c1C(F)(F)F]

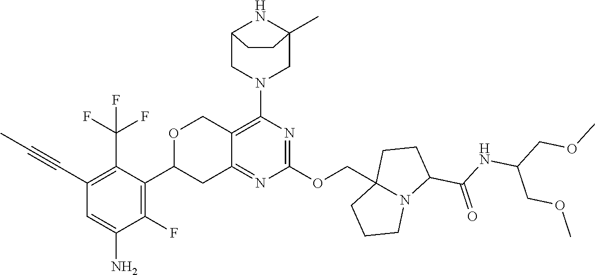 [SMILES: CC#Cc1cc(N)c(F)c(C2Cc3nc(OCC45CCCN4C(C(=O)NC(COC)COC)CC5)nc(N4CC5CCC(C)(C4)N5)c3CO2)c1C(F)(F)F]

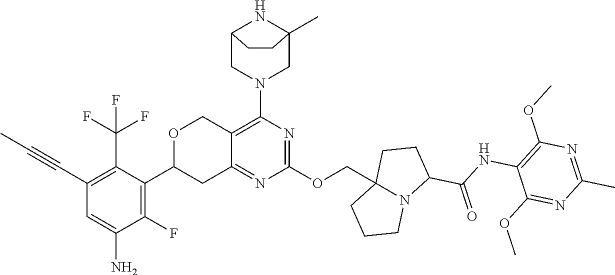 [SMILES: CC#Cc1cc(N)c(F)c(C2Cc3nc(OCC45CCCN4C(C(=O)Nc4c(OC)nc(C)nc4OC)CC5)nc(N4CC5CCC(C)(C4)N5)c3CO2)c1C(F)(F)F]

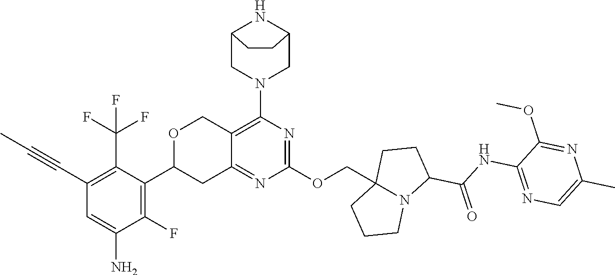 [SMILES: CC#Cc1cc(N)c(F)c(C2Cc3nc(OCC45CCCN4C(C(=O)Nc4ncc(C)nc4OC)CC5)nc(N4CC5CCC(C4)N5)c3CO2)c1C(F)(F)F]

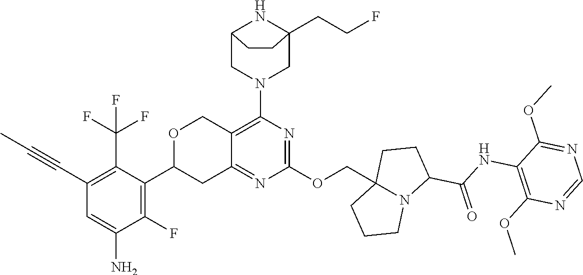 [SMILES: CC#Cc1cc(N)c(F)c(C2Cc3nc(OCC45CCCN4C(C(=O)Nc4c(OC)ncnc4OC)CC5)nc(N4CC5CCC(CCF)(C4)N5)c3CO2)c1C(F)(F)F]

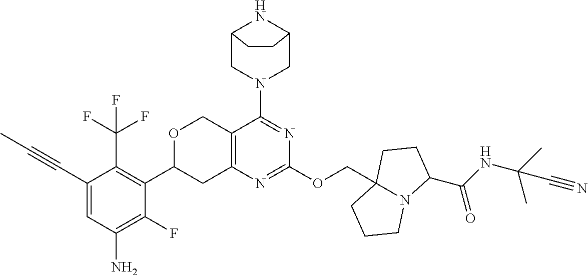 [SMILES: CC#Cc1cc(N)c(F)c(C2Cc3nc(OCC45CCCN4C(C(=O)NC(C)(C)C#N)CC5)nc(N4CC5CCC(C4)N5)c3CO2)c1C(F)(F)F]

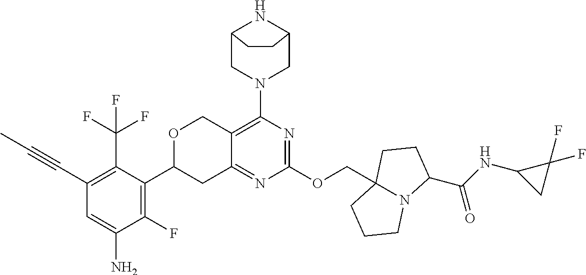 [SMILES: CC#Cc1cc(N)c(F)c(C2Cc3nc(OCC45CCCN4C(C(=O)NC4CC4(F)F)CC5)nc(N4CC5CCC(C4)N5)c3CO2)c1C(F)(F)F]

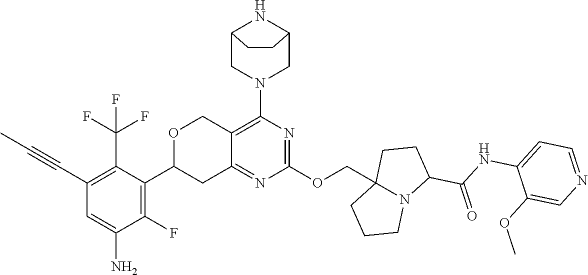 [SMILES: CC#Cc1cc(N)c(F)c(C2Cc3nc(OCC45CCCN4C(C(=O)Nc4ccncc4OC)CC5)nc(N4CC5CCC(C4)N5)c3CO2)c1C(F)(F)F]

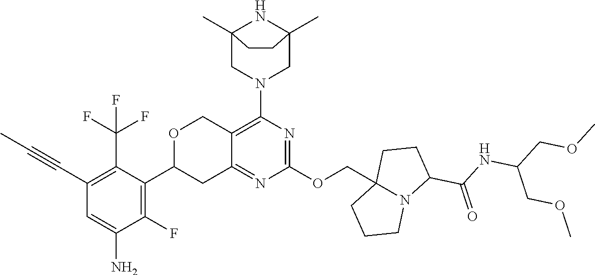 [SMILES: CC#Cc1cc(N)c(F)c(C2Cc3nc(OCC45CCCN4C(C(=O)NC(COC)COC)CC5)nc(N4CC5(C)CCC(C)(C4)N5)c3CO2)c1C(F)(F)F]

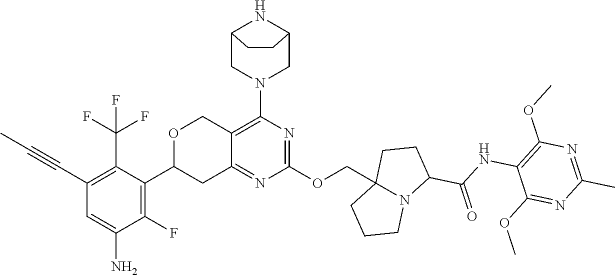 [SMILES: CC#Cc1cc(N)c(F)c(C2Cc3nc(OCC45CCCN4C(C(=O)Nc4c(OC)nc(C)nc4OC)CC5)nc(N4CC5CCC(C4)N5)c3CO2)c1C(F)(F)F]

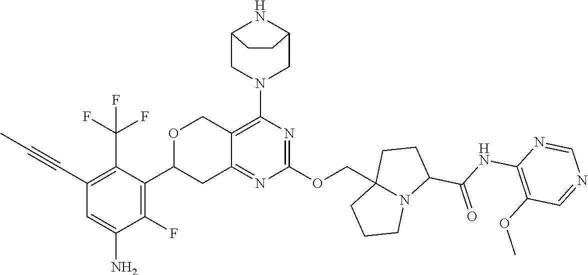 [SMILES: CC#Cc1cc(N)c(F)c(C2Cc3nc(OCC45CCCN4C(C(=O)Nc4ncncc4OC)CC5)nc(N4CC5CCC(C4)N5)c3CO2)c1C(F)(F)F]

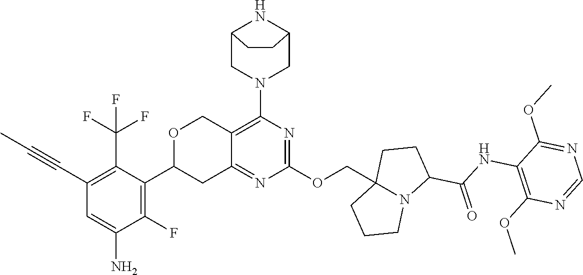 [SMILES: CC#Cc1cc(N)c(F)c(C2Cc3nc(OCC45CCCN4C(C(=O)Nc4c(OC)ncnc4OC)CC5)nc(N4CC5CCC(C4)N5)c3CO2)c1C(F)(F)F]

TABLE 6

| | |
|---|---|
| 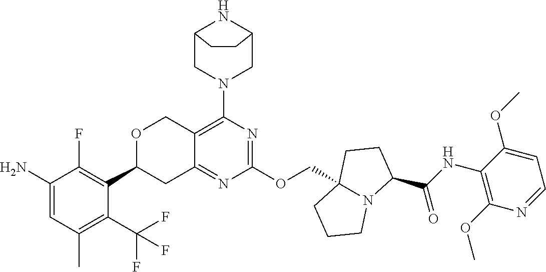 [SMILES: COc1ccnc(OC)c1NC(=O)[C@@H]1CC[C@]2(COc3nc4c(c(N5CC6CCC(C5)N6)n3)CO[C@H](c3c(F)c(N)cc(C)c3C(F)(F)F)C4)CCCN12] | Example S21 |
| 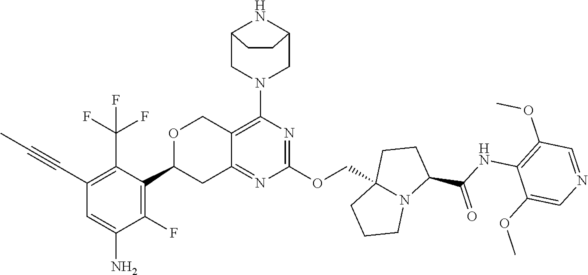 [SMILES: CC#Cc1cc(N)c(F)c([C@@H]2Cc3nc(OC[C@@]45CCCN4[C@H](C(=O)Nc4c(OC)cncc4OC)CC5)nc(N4CC5CCC(C4)N5)c3CO2)c1C(F)(F)F] | Example S77 |
| 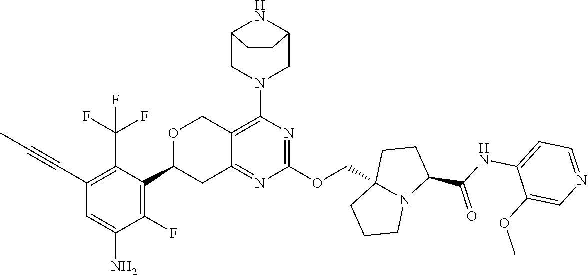 [SMILES: CC#Cc1cc(N)c(F)c([C@@H]2Cc3nc(OC[C@@]45CCCN4[C@H](C(=O)Nc4ccncc4OC)CC5)nc(N4CC5CCC(C4)N5)c3CO2)c1C(F)(F)F] | Example S84 |
| 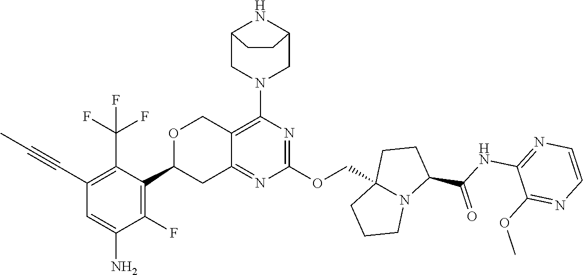 [SMILES: CC#Cc1cc(N)c(F)c([C@@H]2Cc3nc(OC[C@@]45CCCN4[C@H](C(=O)Nc4nccnc4OC)CC5)nc(N4CC5CCC(C4)N5)c3CO2)c1C(F)(F)F] | Example S85 |
| 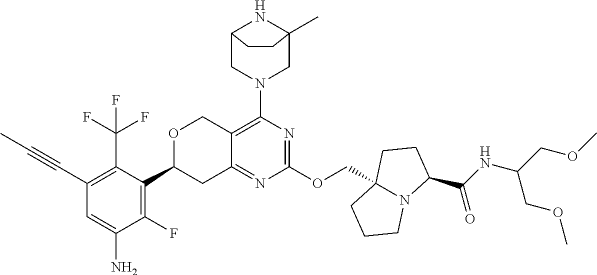 [SMILES: CC#Cc1cc(N)c(F)c([C@@H]2Cc3nc(OC[C@@]45CCCN4[C@H](C(=O)NC(COC)COC)CC5)nc(N4CC5CCC(C)(C4)N5)c3CO2)c1C(F)(F)F] | Example S86A/S86B |
| 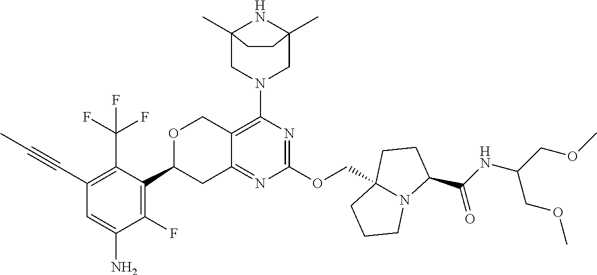 [SMILES: CC#Cc1cc(N)c(F)c([C@@H]2Cc3nc(OC[C@@]45CCCN4[C@H](C(=O)NC(COC)COC)CC5)nc(N4CC5(C)CCC(C)(C4)N5)c3CO2)c1C(F)(F)F] | Example S87 |
| 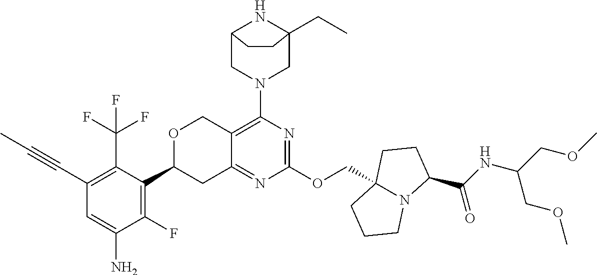 [SMILES: CC#Cc1cc(N)c(F)c([C@@H]2Cc3nc(OC[C@@]45CCCN4[C@H](C(=O)NC(COC)COC)CC5)nc(N4CC5CCC(CC)(C4)N5)c3CO2)c1C(F)(F)F] | Example S88A/S88B |
| 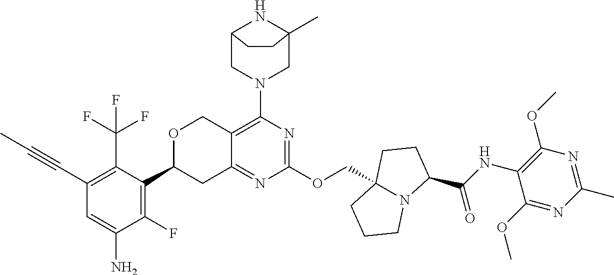 [SMILES: CC#Cc1cc(N)c(F)c([C@@H]2Cc3nc(OC[C@@]45CCCN4[C@H](C(=O)Nc4c(OC)nc(C)nc4OC)CC5)nc(N4CC5CCC(C)(C4)N5)c3CO2)c1C(F)(F)F] | Example S89A/S89B |
| 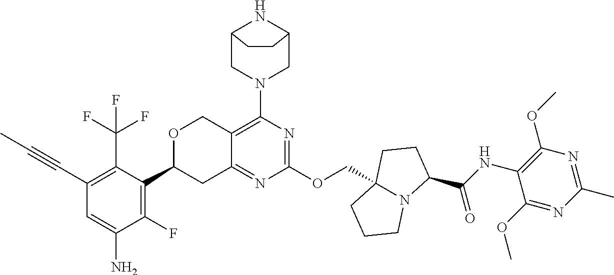 [SMILES: CC#Cc1cc(N)c(F)c([C@@H]2Cc3nc(OC[C@@]45CCCN4[C@H](C(=O)Nc4c(OC)nc(C)nc4OC)CC5)nc(N4CC5CCC(C4)N5)c3CO2)c1C(F)(F)F] | Example S90 |
| 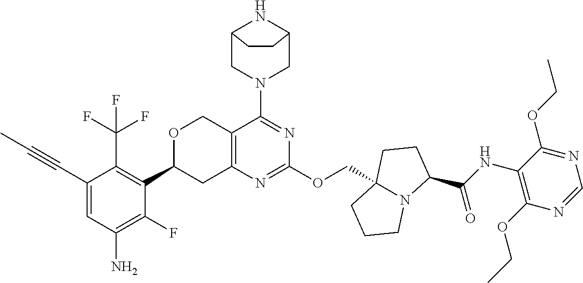 [SMILES: CC#Cc1cc(N)c(F)c([C@@H]2Cc3nc(OC[C@@]45CCCN4[C@H](C(=O)Nc4c(OCC)ncnc4OCC)CC5)nc(N4CC5CCC(C4)N5)c3CO2)c1C(F)(F)F] | Example S91 |
| 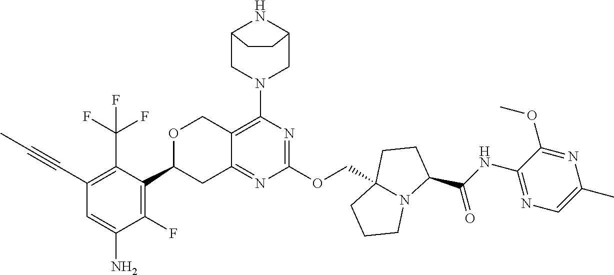 [SMILES: CC#Cc1cc(N)c(F)c([C@@H]2Cc3nc(OC[C@@]45CCCN4[C@H](C(=O)Nc4ncc(C)nc4OC)CC5)nc(N4CC5CCC(C4)N5)c3CO2)c1C(F)(F)F] | Example S92 |
| 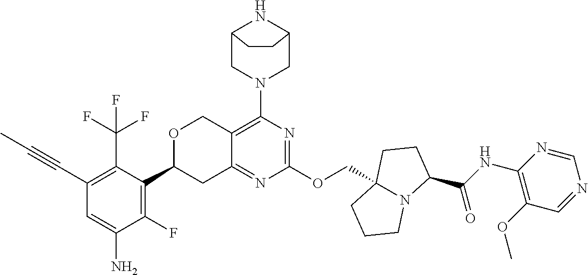 [SMILES: CC#Cc1cc(N)c(F)c([C@@H]2Cc3nc(OC[C@@]45CCCN4[C@H](C(=O)Nc4ncncc4OC)CC5)nc(N4CC5CCC(C4)N5)c3CO2)c1C(F)(F)F] | Example S93 |
| 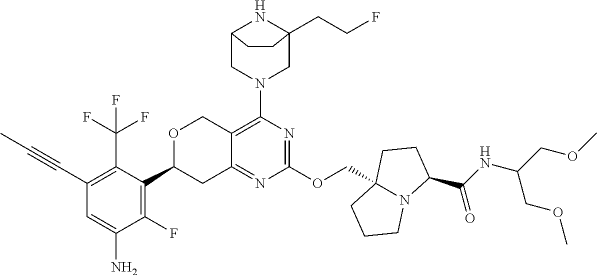 [SMILES: CC#Cc1cc(N)c(F)c([C@@H]2Cc3nc(OC[C@@]45CCCN4[C@H](C(=O)NC(COC)COC)CC5)nc(N4CC5CCC(CCF)(C4)N5)c3CO2)c1C(F)(F)F] | Example S94A/S94B |
| 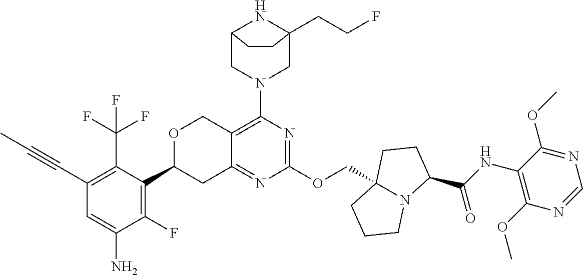 [SMILES: CC#Cc1cc(N)c(F)c([C@@H]2Cc3nc(OC[C@@]45CCCN4[C@H](C(=O)Nc4c(OC)ncnc4OC)CC5)nc(N4CC5CCC(CCF)(C4)N5)c3CO2)c1C(F)(F)F] | Example S95 |
| 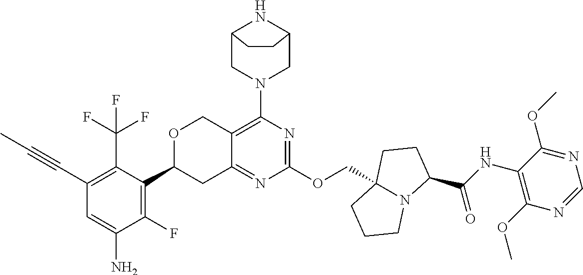 [SMILES: CC#Cc1cc(N)c(F)c([C@@H]2Cc3nc(OC[C@@]45CCCN4[C@H](C(=O)Nc4c(OC)ncnc4OC)CC5)nc(N4CC5CCC(C4)N5)c3CO2)c1C(F)(F)F] | Example S96 |

TABLE 6a

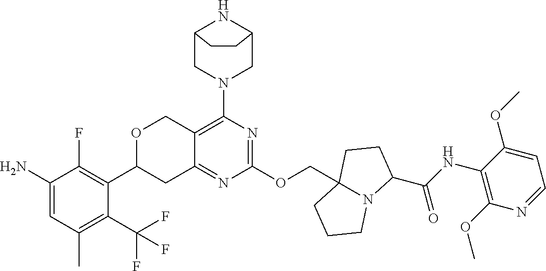 [SMILES: COc1ccnc(OC)c1NC(=O)C1CCC2(COc3nc4c(c(N5CC6CCC(C5)N6)n3)COC(c3c(F)c(N)cc(C)c3C(F)(F)F)C4)CCCN12]

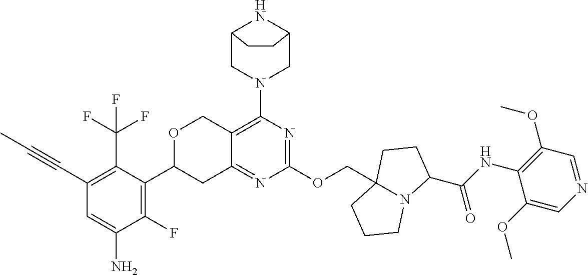 [SMILES: CC#Cc1cc(N)c(F)c(C2Cc3nc(OCC45CCCN4C(C(=O)Nc4c(OC)cncc4OC)CC5)nc(N4CC5CCC(C4)N5)c3CO2)c1C(F)(F)F]

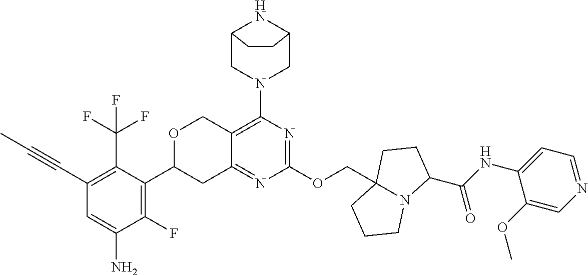 [SMILES: CC#Cc1cc(N)c(F)c(C2Cc3nc(OCC45CCCN4C(C(=O)Nc4ccncc4OC)CC5)nc(N4CC5CCC(C4)N5)c3CO2)c1C(F)(F)F]

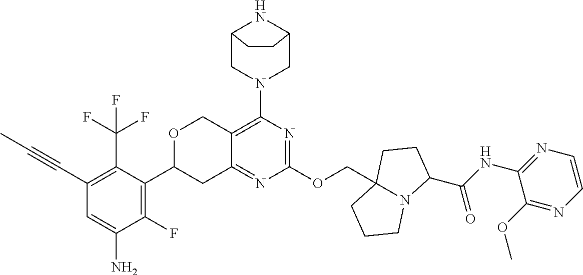 [SMILES: CC#Cc1cc(N)c(F)c(C2Cc3nc(OCC45CCCN4C(C(=O)Nc4nccnc4OC)CC5)nc(N4CC5CCC(C4)N5)c3CO2)c1C(F)(F)F]

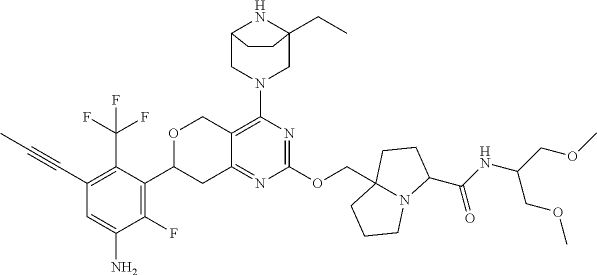 [SMILES: CC#Cc1cc(N)c(F)c(C2Cc3nc(OCC45CCCN4C(C(=O)NC(COC)COC)CC5)nc(N4CC5CCC(CC)(C4)N5)c3CO2)c1C(F)(F)F]

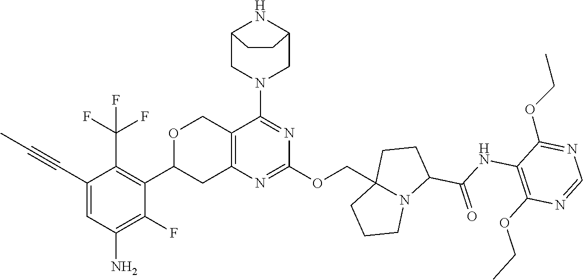 [SMILES: CC#Cc1cc(N)c(F)c(C2Cc3nc(OCC45CCCN4C(C(=O)Nc4c(OCC)ncnc4OCC)CC5)nc(N4CC5CCC(C4)N5)c3CO2)c1C(F)(F)F]

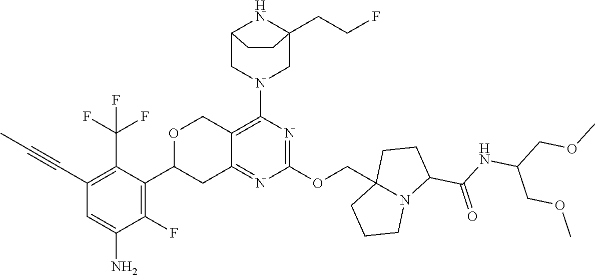 [SMILES: CC#Cc1cc(N)c(F)c(C2Cc3nc(OCC45CCCN4C(C(=O)NC(COC)COC)CC5)nc(N4CC5CCC(CCF)(C4)N5)c3CO2)c1C(F)(F)F]

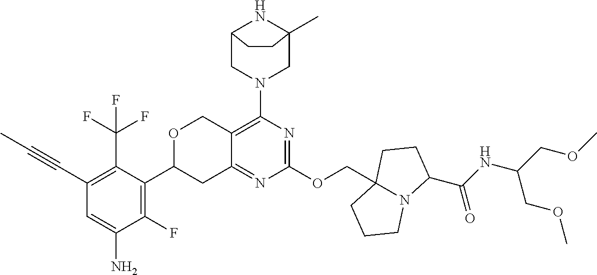 [SMILES: CC#Cc1cc(N)c(F)c(C2Cc3nc(OCC45CCCN4C(C(=O)NC(COC)COC)CC5)nc(N4CC5CCC(C)(C4)N5)c3CO2)c1C(F)(F)F]

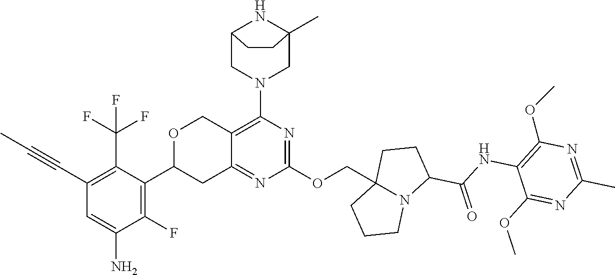 [SMILES: CC#Cc1cc(N)c(F)c(C2Cc3nc(OCC45CCCN4C(C(=O)Nc4c(OC)nc(C)nc4OC)CC5)nc(N4CC5CCC(C)(C4)N5)c3CO2)c1C(F)(F)F]

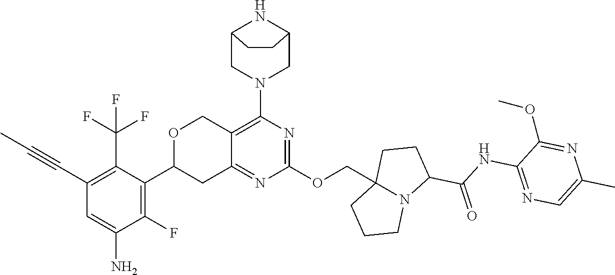 [SMILES: CC#Cc1cc(N)c(F)c(C2Cc3nc(OCC45CCCN4C(C(=O)Nc4ncc(C)nc4OC)CC5)nc(N4CC5CCC(C4)N5)c3CO2)c1C(F)(F)F]

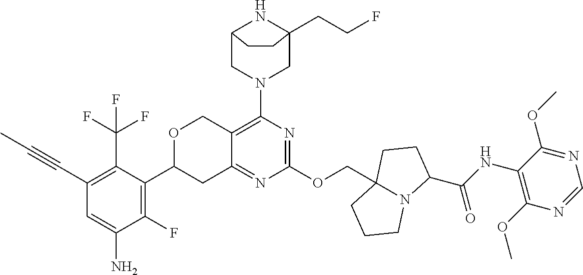 [SMILES: CC#Cc1cc(N)c(F)c(C2Cc3nc(OCC45CCCN4C(C(=O)Nc4c(OC)ncnc4OC)CC5)nc(N4CC5CCC(CCF)(C4)N5)c3CO2)c1C(F)(F)F]

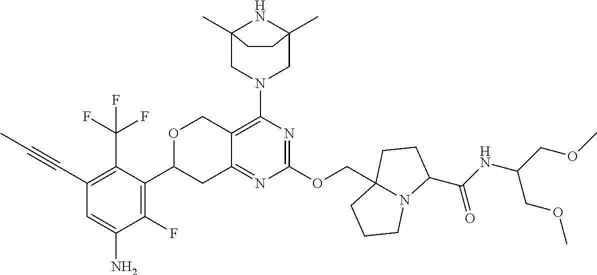 [SMILES: CC#Cc1cc(N)c(F)c(C2Cc3nc(OCC45CCCN4C(C(=O)NC(COC)COC)CC5)nc(N4CC5(C)CCC(C)(C4)N5)c3CO2)c1C(F)(F)F]

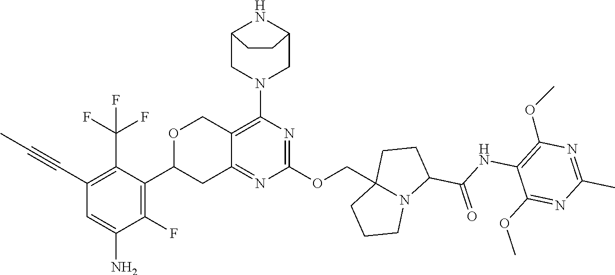 [SMILES: CC#Cc1cc(N)c(F)c(C2Cc3nc(OCC45CCCN4C(C(=O)Nc4c(OC)nc(C)nc4OC)CC5)nc(N4CC5CCC(C4)N5)c3CO2)c1C(F)(F)F]

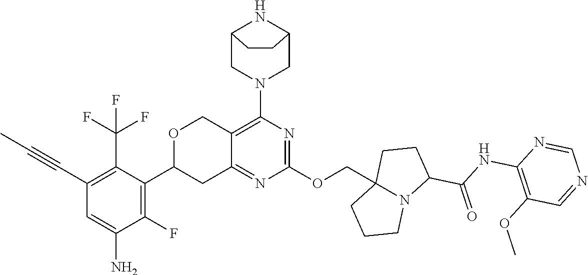 [SMILES: CC#Cc1cc(N)c(F)c(C2Cc3nc(OCC45CCCN4C(C(=O)Nc4ncncc4OC)CC5)nc(N4CC5CCC(C4)N5)c3CO2)c1C(F)(F)F]

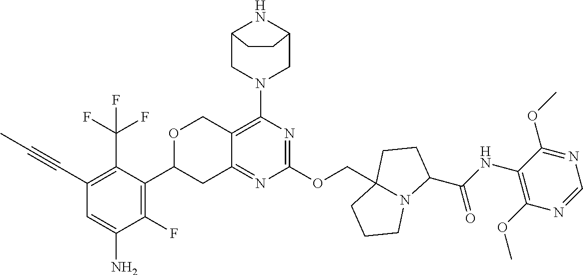 [SMILES: CC#Cc1cc(N)c(F)c(C2Cc3nc(OCC45CCCN4C(C(=O)Nc4c(OC)ncnc4OC)CC5)nc(N4CC5CCC(C4)N5)c3CO2)c1C(F)(F)F]

In some embodiments, the compound of the present disclosure is a compound selected from Table 1, a stereoisomer thereof, or a pharmaceutically acceptable salt thereof.

In some embodiments, the compound of the present disclosure is a compound selected from Table 2, a stereoisomer thereof, or a pharmaceutically acceptable salt thereof.

In some embodiments, the compound of the present disclosure is a compound selected from Table 2a, a stereoisomer thereof, or a pharmaceutically acceptable salt thereof.

In some embodiments, the compound of the present disclosure is a compound selected from Table 3, a stereoisomer thereof, or a pharmaceutically acceptable salt thereof.

In some embodiments, the compound of the present disclosure is a compound selected from Table 4, a stereoisomer thereof, or a pharmaceutically acceptable salt thereof.

In some embodiments, the compound of the present disclosure is a compound selected from Table 5, a stereoisomer thereof, or a pharmaceutically acceptable salt thereof.

In some embodiments, the compound of the present disclosure is a compound selected from Table 5a, a stereoisomer thereof, or a pharmaceutically acceptable salt thereof.

In some embodiments, the compound of the present disclosure is a compound selected from Table 6, a stereoisomer thereof, or a pharmaceutically acceptable salt thereof.

In some embodiments, the compound of the present disclosure is a compound selected from Table 6a, a stereoisomer thereof, or a pharmaceutically acceptable salt thereof.

In certain aspects, the present disclosure provides pharmaceutical compositions comprising a compound disclosed herein or a stereoisomer thereof, or a pharmaceutically acceptable salt thereof, and a pharmaceutically acceptable excipient.

In certain aspects, the present disclosure provides methods of treating or preventing a disease or disorder (e.g., a disease or disorder associated with dysregulation and/or mutation of KRAS) in a subject in need thereof, comprising administering to the subject a compound disclosed herein or a stereoisomer thereof, or a pharmaceutically acceptable salt thereof (e g., in a therapeutically effective amount).

In certain aspects, the present disclosure provides methods of treating a disease or disorder (e.g., a disease or disorder associated with dysregulation and/or mutation of KRAS) in a subject in need thereof, comprising administering to the subject a compound disclosed herein or a stereoisomer thereof, or a pharmaceutically acceptable salt thereof (e.g., in a therapeutically effective amount).

In certain aspects, the present disclosure provides uses of a compound disclosed herein or a stereoisomer thereof, or a pharmaceutically acceptable salt thereof in the manufacture of a medicament for treating or preventing a disease or disorder (e.g., a disease or disorder associated with dysregulation and/or mutation of KRAS) in a subject in need thereof.

In certain aspects, the present disclosure provides uses of a compound disclosed herein or a stereoisomer thereof, or a pharmaceutically acceptable salt thereof in the manufacture of a medicament for treating a disease or disorder (e.g., a disease or disorder associated with dysregulation and/or mutation of KRAS) in a subject in need thereof.

### Definitions

The details of the disclosure are set forth in the accompanying description below. Although methods and materials similar or equivalent to those described herein can be used in the practice or testing of the present disclosure, illustrative methods and materials are now described. Other features, objects, and advantages of the disclosure will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this disclosure belongs. All patents and publications cited in this specification are incorporated herein by reference in their entireties.

Unless otherwise specified, the following terms and phrases used herein are intended to have the following meanings. A particular term or phrase should not be considered indeterminate or unclear unless specifically defined, but should be understood in its ordinary meaning. When a trade name appears herein, it is intended to refer to its corresponding commercial product or its active ingredient.

The articles “a” and “an” are used in this disclosure to refer to one or more than one (i.e., at least one) of the grammatical object of the article. By way of example, “an element” means one element or more than one element.

The term “and/or” is used in this disclosure to mean either “and” or “or” unless indicated otherwise.

The term “pharmaceutically acceptable” is used herein for those compounds, materials, compositions, and/or dosage forms which are, within the scope of sound medical judgment, suitable for use in contact with the tissues of human beings and animals without excessive toxicity, irritation, allergic response, or other problems or complications, and commensurate with a reasonable benefit/risk ratio.

The term “pharmaceutically acceptable salt” refers to a salt of the compound disclosed herein, which is prepared from the compound having particular substituents disclosed herein and a relatively non-toxic acid or base. When the compound of the present disclosure contains a relatively acidic functional group, a base addition salt may be obtained by contacting such a compound with a sufficient amount of a base in a pure solution or a suitable inert solvent. When the compound of the present disclosure contains a relatively basic functional group, an acid addition salt may be obtained by contacting such a compound with a sufficient amount of an acid in a pure solution or a suitable inert solvent. Certain specific compounds disclosed herein contain both basic and acidic functional groups that allow the compounds to be converted into either base or acid addition salts.

The pharmaceutically acceptable salts of the present disclosure may be synthesized from a parent compound containing acid radicals or bases by means of conventional chemical methods. In general, such salts are prepared by the following method: the free acid or base form of the compound reacting with a stoichiometric amount of the appropriate base or acid in water or an organic solvent or a mixture thereof.

The compound of the present disclosure may have a specific geometric or stereoisomeric form. All such compounds are contemplated herein, including cis and trans isomers, (−)- and (+)-enantiomers, (R)- and(S)-enantiomers, diastereomers, (D)-isomers, (L)-isomers, and racemic mixtures and other mixtures thereof, such as enantiomer or diastereomer enriched mixtures, all of which are encompassed within the scope of the present disclosure. Substituents such as alkyl may have an additional asymmetric carbon atom. All these isomers and mixtures thereof are encompassed within the scope of the present disclosure.

The compound disclosed herein may contain an unnatural proportion of atomic isotope at one or more of the atoms that constitute the compound. For example, the compound may be labeled with a radioisotope, such as tritium (³H), iodine-125 (¹²⁵I), or C-14 (¹⁴C) Citing another example, hydrogen may be substituted by deuterium to form a deuterated drug, and the bond formed by deuterium and carbon is firmer than that formed by common hydrogen and carbon. Compared with an un-deuterated drug, the deuterated drug has the advantages of reduced toxic side effect, increased stability, enhanced efficacy, prolonged biological half-life and the like. All isotopic variations of the compound described herein, whether radioactive or not, are encompassed within the scope of the present disclosure.

The terms of “optional” or “optionally” means that the subsequently described event or circumstance may, but not necessarily, occur, and the description includes instances where the event or circumstance occurs and instances where it does not.

The term “substituted” means that one or more hydrogen atoms on a specific atom are substituted by substituent(s), of which the substituent group may include deuterium and hydrogen variants, as long as the valence of the specific atom is normal and the substituted compound is stable. When the substituent is an oxygen or oxo (i.e., ═O), it means that two hydrogen atoms are substituted. The term “optionally substituted” means that an atom may or may not be substituted Unless otherwise specified, the type and number of the substituents may be arbitrary as long as being chemically achievable.

When any variable (e.g., R) occurs more than once in the constitution or structure of a compound, the definition of the variable in each case is independent. Thus, for example, if a group is substituted by 0-2 R, the group may be optionally substituted by two R at most, and the definition of R in each case is independent. Furthermore, a combination of a substituent and/or a variant thereof is permissible only if the combination can result in a stable compound.

When the number of a linking group is 0, such as —(CRR)₀—, it means that the linking group is a single bond.

When one of the variables is selected from a single bond, it means that the two groups to which it is connected are directly connected. For example, when L represents a single bond in A-L-Z, it means that the structure is actually A-Z.

When the listed linking group does not indicate its connection direction, its connection direction is arbitrary, for example, the middle linking group L in

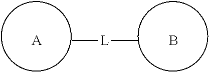

is -M-W—, at which point -M-W— can be composed of ring A and ring B in the same direction as the reading order from left to right

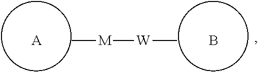

or ring A and ring B in the opposite direction as the reading order from left to right

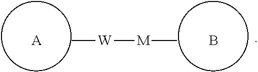

A combination of the linking group, a substituent and/or a variant thereof is permissible only if the combination can result in a stable compound.

Unless otherwise specified, when a group has one or more linkable sites, any one or more sites of the group may be linked to other groups by a chemical bond. When the chemical bond is attached in a manner that is not localized, and the H atom is present at the linkable site, the number of H atoms at the site is correspondingly reduced to a corresponding valency group with the number of chemical bonds attached. The chemical bond to which the site is attached to other groups may be represented by a straight solid bond ( [symbol] ), a straight dotted bond ( [symbol] ), or a wavy line ( [symbol] ). For example, a straight solid bond in-OCH; indicates that the oxygen atoms in the group are connected to other groups; a straight dotted bond in indicates that the nitrogen atoms at both ends in the group are connected to other groups; a wavy line in indicates that the carbon atoms at positions 1 and 2 in the phenyl group are connected to other groups; indicates that any attachable site on the piperidine group can be connected to other groups by at least 1 chemical bond, including

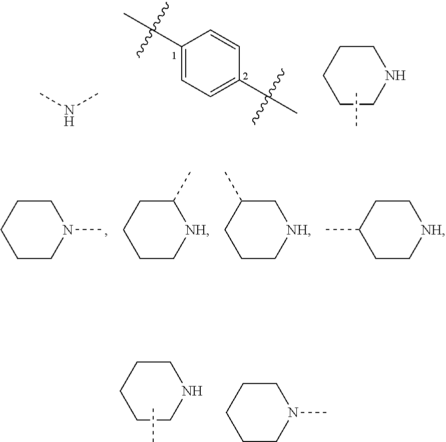

at least 4 ways of attachment. Even if H atoms are drawn on —N—, the group in this connection mode is still included, but when 1 chemical bond is connected, the H of the site will correspondingly reduce 1 to the corresponding monovalent piperidine group.

Unless otherwise stated, the absolute configuration of a stereogenic center is represented by a wedged solid bond ( [symbol] ) and a wedged dashed bond (cid: [symbol] ) and the relative configuration of a stereogenic center is represented by a straight solid bond ( [symbol] ) and a straight dashed bond ( [symbol] ). A wavy line ( [symbol] ) represents a wedged solid bond ( [symbol] ) or a wedged dashed bond ( [symbol] ), or a wavy line ( [symbol] ) represents a straight solid bond (cid: [symbol] ) or a straight dashed bond ( [symbol] ).

Unless otherwise stated, when a double bond structure is present in a compound, such as a carbon double bond, a carbon nitrogen double bond, and a nitrogen double bond, and each atom on the double bond is connected with two different substituents (in a double bond comprising a nitrogen atom, a pair of lone pairs of electrons on the nitrogen atom are considered one substituent to which it is connected), a mixture of two isomers of the compound is represented if the atom on the double bond is

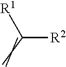

represented between the atom and the substituent in the compound.

Unless otherwise stated, the term “tautomer” or “tautomeric form” means that different functional isomers are in dynamic equilibrium at room temperature and may be rapidly converted into each other. Where tautomerization is possible (e.g., in solution), the chemical equilibrium of tautomers may be achieved. For example, a proton tautomer (also known as a prototropic tautomer) includes the interconversion by proton transfer, such as keto-enol isomerization and imine-enamine isomerization. A valence isomer includes the interconversion by recombination of some bonding electrons. A specific example of the keto-enol tautomerization is the interconversion between tautomers pentane-2,4-dione and 4-hydroxypent-3-en-2-one.

Unless otherwise stated, the term “enriched with one isomer”, “isomer enriched”, “enriched with one enantiomer”, or “enantiomer enriched” means that the content of one of the isomers or enantiomers is less than 100% and more than or equal to 60%, 70%, 80%, 90%, 95%, 96%, 97%, 98%, 99%, 99.5%, 99.6%, 99.7%, 99.8%, or 99.9%.

Unless otherwise stated, the term “isomeric excess” or “enantiomeric excess” refers to the difference between the relative percentages of two isomers or enantiomers. For example, if the content of one isomer or enantiomer is 90% and the content of the other isomer or enantiomer is 10%, the isomeric or enantiomeric excess (ee value) is 80%.

Unless otherwise stated, Cₙ₋ₙ₊ₘ or Cₙ-Cₙ₊ₘ includes any one of the specific cases of n to n+m carbon atoms. For example, C₁₋₁₂ includes C₁, C₂, C₃, C₄, C₅, C₆, C₇, C₈, C₉, C₁₀, C₁₁ and C₁₂. Also, any range within n to n+m may be included. For example, C₁₋₁₂ includes C₁₋₂, C₁₋₆, C₁₋₉, C₃₋₆, C₃₋₉, C₃₋₁₂, C₆₋₉, C₆₋₁₂ and C₉₋₁₂, etc. Similarly, n-n+m membered means that the number of atoms on the ring is n to n+m. For example, 3-12 membered ring includes 3 membered ring, 4 membered ring, 5 membered ring, 6 membered ring, 7 membered ring, 8 membered ring, 9 membered ring, 10 membered ring, 11 membered ring and 12 membered ring. n-n+m membered also represents any range within n to n+m. For example, 3-12 membered ring includes 3-6 membered ring, 3-9 membered ring, 5-6 membered ring. 5-7 membered ring, 6-7 membered ring, 6-8 membered ring, 6-10 membered ring, etc.

The term “halo” or “halogen,” by itself or as part of another substituent, means, unless otherwise stated, a fluorine, chlorine, bromine, or iodine atom.

Unless otherwise stated, the term “C₁₋ₙ alkyl” refers to a straight-chained or branched saturated hydrocarbon group consisting of 1 to n (e.g., 4, 5, or 6) carbon atoms. For example, when n is 6, C₁₋ₙ alkyl includes C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, C₅₋₆, C₆, C₅, C₄, C₃, C₂, and C₁ alkyl, etc. Examples of C₁₋₆ alkyl include, but are not limited to, methyl (Me), ethyl (Et), propyl (including n-propyl and i-propyl), butyl (including n-butyl, i-butyl, s-butyl, and t-butyl), pentyl (including n-pentyl and i-pentyl), hexyl, and the like.

Unless otherwise specified, the term “C₁₋ₙ alkoxy” or “C₁₋ₙ alkyoxy” denotes alkyl groups containing 1 to n (e.g., 4, 5, or 6) carbon atoms that are attached to the remainder of the molecule by one oxygen atom. For example, when n is 6, C₁₋ₙ alkoxy includes C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, C₅₋₆, C₆, C₅, C₄, C₃, C₂, and C₁ alkoxy, etc. Examples of C₁₋₆ alkoxy include, but are not limited to, methoxy, ethoxy, propoxy (including n-propoxy and i-propoxy), butoxy (including n-butoxy, i-butoxy, s-butoxy, and t-butoxy), pentoxy (including n-pentoxy and i-pentoxy), hexyloxy, and the like.

Unless otherwise specified, the term “C₁₋ₙ alkylamino” denotes —NH—C₁₋ₙ alkyl groups. For example, when n is 3. C₁₋ₙ alkylamino include, but are not limited to, —NHCH₃, —NHCH₂CH₃, —NHCH₂CH₂CH₃, —NHCH₂ (CH₃)₂, etc.

Unless otherwise specified, the term “di-C₁₋ₙ alkylamino” denotes-N(C₁₋ₙ alkyl)₂ groups. For example, when n is 3, di-C₁₋ₙ alkylamino include, but are not limited to, —N(CH₃)₂ and —N(CH₃) CH₂CH₃.

Unless otherwise stated, “C₂₋ₙ alkenyl” refers to a straight-chained or branched hydrocarbon group consisting of 2 to n carbon atoms containing at least one carbon-carbon double bond, which may be located anywhere in the group. For example, when n is 4, C₂₋ₙ alkenyl group includes C₂₋₄, C₂₋₃, C₃₋₄, C₄, C₃, and C₂ alkenyl, etc. Examples of C₂₋₄ alkenyl include, but are not limited to, vinyl, allyl, butenyl, butadienyl, and the like.

Unless otherwise stated, “C₂₋ₙ alkynyl” refers to a straight-chained or branched hydrocarbon group consisting of 2 to n carbon atoms containing at least one carbon-carbon triple bond, which may be located anywhere in the group. For example, when n is 4, C₂₋ₙ alkynyl groups include C₂₋₄, C₂₋₃, C₃₋₄, C₄, C₃, and C₂ alkynyl, etc. Examples of C₂₋₄ alkynyl include, but are not limited to, ethynyl, propynyl, butynyl, and the like.

Unless otherwise specified, “C₃₋ₙ cycloalkyl” refers to a saturated cyclic hydrocarbon group consisting of 3 to n carbon atoms that includes a monocyclic, bicyclic, and tricyclic system, wherein the bicyclic and tricyclic systems include a spiro ring, a parallel ring, and a bridging ring. When n is 10, C₃₋ₙ cycloalkyl includes C₃₋₁₀, C₃₋₉, C₃₋₈, C₃₋₇, C₃₋₆, C₃₋₅, C₃₋₄, C₄₋₁₀, C₄₋₉, C₄₋₈, C₄₋₇, C₄₋₆, C₄₋₅, . . . C₉₋₁₀, C₁₀, C₉, C₈, C₇, C₆, C₅, C₄, etc. Examples of C₃₋₁₀ cycloalkyl include, but are not limited to, cyclopropyl, cyclobutyl, cyclopentyl, cyclohexyl, cycloheptyl, ice-lowering alkyl, [2.2.2]dicyclooctane etc.

Unless otherwise specified, the term “heterocyclyl” denotes a saturated or partially unsaturated cyclic group, of which one or more ring atoms being heteroatoms independently selected from O, S, and N, the remaining being carbon atoms, wherein the carbon atoms are optionally substituted with oxygen (i.e., C(O)), the nitrogen atoms are optionally quaternized, the nitrogen and sulfur heteroatoms can optionally be oxidized (i.e., NO and S(O)ₚ, p is 1 or 2). “Heterocyclyl” may be monocyclic or multicyclic (including spiro-, bridged-, and fused-ring system), and may comprise 3- to 10-membered ring or rings. Examples of heterocyclyl groups include, but are not limited to, azetidyl, oxetyl, thiobutyl, pyrrolidinyl, pyrazolyl, imidazoalkyl, tetrahydrothienyl (including tetrahydrothien-2-yl and tetrahydrothien-3-yl, etc.), tetrahydrofuranyl (including tetrahydrofuran-2-yl, etc.), piperidinyl (including 1-piperidinyl, 2-piperidinyl and 3-piperidinyl, etc.), piperazinyl (including 1-piperazinyl and 2-piperazinyl etc.), morpholinyl (including 3-morpholinyl and 4-morpholinyl, etc.), dioxanyl, dithianyl, isooxazolidyl, isothiazolidyl, 1,2-oxazinyl, 1, 2-thiazinyl, or hexahydropyridazinyl, or the like. In the case of multicyclic heterocyclyl, only one of the rings in the heterocyclyl needs to be non-aromatic (e.g., 4,5,6,7-tetrahydrobenzo[c]isoxazolyl, benzo[d][1,3]dioxole, 2,3-dihydrobenzo[b][1,4]dioxine, 2,3-dihydrobenzofuran, and the like).

Unless otherwise specified, the terms “C₆₋₁₀ aromatic ring” and “C₆₋₁₀ aryl” are used interchangeably, and refer to a cyclic hydrocarbon group having a conjugated π electron system consisting of 6 to 10 carbon atoms, which may be a monocyclic, fused bicyclic, or fused tricyclic system, wherein each ring is aromatic. C₆₋₁₀ aryl includes C₆₋₉, C₉, C₁₀, and C₆ aryl etc. Examples of C₆₋₁₀ aryl groups include, but are not limited to, phenyl, naphthyl (including 1-naphthyl and 2-naphthyl, etc.).

Unless otherwise specified, the terms “heteroaryl ring” and “heteroaryl” are used interchangeably, and refer to a cyclic group representing a conjugated π electron system and may comprise 5-10 membered ring or rings, of which one or more ring atoms are heteroatoms independently selected from O, S, and N, and the rest of which are carbon atoms. It can be a monocyclic, fused bicyclic, or fused tricyclic system comprising one, two, or more rings, where each ring is aromatic, wherein the nitrogen atoms are optionally quaternized, the nitrogen and sulfur heteroatoms could optionally be oxidized (i.e. NO and S(O)ₚ, p is 1 or 2). Examples of heteroaryl include, but are not limited to, pyrrolyl (including N-pyrrolyl, 2-pyrrolyl, and 3-pyrrolyl, etc.), pyrazolyl (including 2-pyrazolyl and 3-pyrazolyl, etc.), imidazolyl (including N-imidazolyl, 2-imidazolyl, 4-imidazolyl, and 5-imidazolyl, etc.), oxazolyl (including 2-oxazolyl, 4-oxazolyl, and 5-oxazolyl, etc.), triazolyl 1H-1,2,3-triazolyl, 2H-1,2,3-triazolyl, 1H-1,2,4-triazolyl and 4H-1,2,4-triazoly etc.), tetrazolyl, isoxazolyl (3-isoxazolyl, 4-isoxazolyl and 5-isoxazolyl etc.), thiazolyl (including 2-thiazolyl, 4-thiazolyl and 5-thiazolyl etc.), furanyl (including 2-furanyl and 3-furanyl, etc.), thiophenyl (including 2-thiophenyl and 3-thiophenyl, etc.), pyridinyl (including 2-pyridinyl, 3-pyridinyl and 4-pyridinyl, etc.), pyrazinyl, pyridinyl (including 2-pyridinyl and 4-pyridinyl, etc.), benzothiazolyl (including 5-benzothiazolyl, etc.), purinyl, benzimidazolyl (including 2-benzoimidazolyl, etc.), benzoxazolyl, indolyl (including 5-indolyl, etc.), isoquinolinyl (including 1-isoquinolinyl and S-isoquinolinyl, etc.), quinoxolinyl (including 2-quinoxolinyl and 5-quinoxolinyl, etc.), or quinolinyl (including 3-quinoline and 6-quinoline, etc.), or the like.

The compound of the present disclosure may be prepared by a variety of synthetic methods well known to those skilled in the art, including the following enumerative embodiment, the embodiment formed by the following enumerative embodiment in combination with other chemical synthesis methods and the equivalent replacement well known to those skilled in the art. The preferred embodiment includes, but is not limited to the embodiment of the present disclosure.

The structure of the compound of the present disclosure may be confirmed by conventional methods well known to those skilled in the art, and if the present disclosure relates to the absolute configuration of the compound, the absolute configuration may be confirmed by conventional technical means in the art. For example, single crystal X-ray diffraction (SXRD) may be used, wherein a cultured single crystal is analyzed by a Bruker D8 Venture diffractometer to collect diffraction intensity data, and the light source is CuKα radiation, and the scanning mode is: φ/ω scanning; and after relevant data are collected, the crystal structure is further analyzed by using a direct method (Shelxs97), so that the absolute configuration can be confirmed.

The solvents used in the present disclosure are commercially available. The present disclosure uses the following abbreviations: NaOH stands for sodium hydroxide; DMF stands for N,N-dimethylformamide; THF stands for tetrahydrofuran; 2-MeTHF stands for 2-methyl tetrahydrofuran; DCM stands for dioxane; EA stands for ethyl acetate; DIPEA stands for N,N-diisopropylethylamine; DCM stands for dichloroperoxybenzoic acid; m-CPBA stands for m-chloroperoxybenzoic acid; Boc₂O stands for di-tert-butyl carbonic anhydride; LiAlH₄ stands for lithium tetrahydroxium; MnO₂ stands for manganese dioxide; NBS stands for N-bromosuccinimide, TMP stands for trimethylpropane; n-BuLi stands for n-butyl lithium; TFA stands for trifluoroacetic acid; Xphos Pd G4 stands for: (SP-4-3) [dicyclohexyl [2′,4′,6′-tris (isopropyl) [1,1′-biphenyl]-2-yl]phosphine](methanesulfonic acid) [2′-(methylamino) [1, l′-biphenyl]-2-yl]palladium; AgNO; stands for silver nitrate; NCS stands for N-chlorosuccinimide.

Compounds are named according to conventional nomenclature in the art or using ChemDraw® software, and supplier's catalog names are adopted for commercially available compounds.

The term “administer”, “administering”, or “administration” as used in this disclosure refers to either directly administering a disclosed compound or a stereoisomer thereof, or a pharmaceutically acceptable salt thereof or a composition to a subject, which can form an equivalent amount of active compound within the subject's body.

A “subject” is a mammal, e.g., a human, mouse, rat, guinea pig, dog, cat, horse, cow, pig, or non-human primate, such as a monkey, chimpanzee, baboon or rhesus.

An “effective amount” or “therapeutically effective amount” when used in connection with a compound or a stereoisomer thereof, or a pharmaceutically acceptable salt thereof or a pharmaceutical composition is an amount effective for treating or preventing a disease in a subject as described herein.

The term “treating” with regard to a subject, refers to improving at least one symptom of the subject's disorder. Treating includes curing, improving, or at least partially ameliorating the disorder.

The compounds of the present disclosure, or a pharmaceutically acceptable salt, thereof, can also be used to prevent a disease, condition or disorder. As used herein, “preventing” or “prevent” describes reducing or eliminating the onset of the symptoms or complications of the disease, condition or disorder.

The term “disorder” is used in this disclosure to mean, and is used interchangeably with, the terms disease, condition, or illness, unless otherwise indicated.

As used herein, a KRAS mutation-associated disease or disorder, or a disease or disorder associated with dysregulation and/or mutation of KRAS means any disease or other deleterious condition in which a mutation of or dysregulation or disruption of the function of KRAS plays a role. Accordingly, another embodiment of the present disclosure relates to treating or lessening the severity of one or more diseases in which a mutation of or dysregulation or disruption of the function of KRAS plays a role. Specifically, the present disclosure relates to a method of treating or lessening the severity of a disease or condition as described herein, wherein said method comprises administering to a subject in need thereof a compounds of the present disclosure (e.g., a compound of any of the formulae or any individual compounds disclosed herein), or pharmaceutically acceptable salts, solvates, prodrugs, stereoisomers, or tautomers thereof, or a composition according to the present disclosure.

In some embodiments, the KRAS mutation is a KRAS G12D, G12C, or G12V mutation.

In some embodiments, the KRAS mutation is a KRAS G12D mutation.

In some embodiments, the KRAS mutation is a KRAS G12C mutation.

In some embodiments, the KRAS mutation is a KRAS G12V mutation.

The present disclosure also provides preparation of drugs for treating KRAS mutation-associated diseases or disorders, using the compounds or stereoisomers thereof, or pharmaceutically acceptable salts thereof disclosed herein.

The present disclosure also provides the use of the compounds or stereoisomers thereof, or pharmaceutically acceptable salts thereof disclosed herein for the treatment of a KRAS mutation-associated disease or disorder.

The present disclosure also provides the compounds or stereoisomers thereof, or pharmaceutically acceptable salts thereof disclosed herein for use in the manufacture of a medicament for the treatment of a KRAS mutation-associated disease or disorder.

The present disclosure also provides a method of treating a disease or disorder associated with a KRAS mutation, comprising administering to a subject in need thereof the compounds or stereoisomers thereof, or pharmaceutically acceptable salts thereof disclosed herein.

The present disclosure also provides a pharmaceutical composition comprising a compound of the present disclosure (e.g., a compound of any of the formulae or any individual compounds disclosed herein) or a stereoisomer thereof, or a pharmaceutically acceptable salt thereof, and at least one pharmaceutically acceptable excipient or carrier.

A “pharmaceutical composition” is a formulation containing the compound of the present disclosure in a form suitable for administration to a subject. In some embodiments, the pharmaceutical composition is in bulk or in unit dosage form. The unit dosage form is any of a variety of forms, including, for example, a capsule, an IV bag, a tablet, a single pump on an aerosol inhaler or a vial. The quantity of active ingredient (e.g., a formulation of the disclosed compound or a stereoisomer thereof, or a pharmaceutically acceptable salt thereof) in a unit dose of composition is an effective amount and is varied according to the particular treatment involved. One skilled in the art will appreciate that it is sometimes necessary to make routine variations to the dosage depending on the age and condition of the patient. The dosage will also depend on the route of administration. A variety of routes are contemplated, including oral, pulmonary, rectal, parenteral, transdermal, subcutaneous, intravenous, intramuscular, intraperitoneal, inhalational, buccal, sublingual, intrapleural, intrathecal, intranasal, and the like. Dosage forms for the topical or transdermal administration of a compound of this disclosure include powders, sprays, ointments, pastes, creams, lotions, gels, solutions, patches and inhalants. In some embodiments, the active compound is mixed under sterile conditions with a pharmaceutically acceptable carrier, and with any preservatives, buffers or propellants that are required.

As used herein, the phrase “pharmaceutically acceptable” refers to those compounds, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used in the specification and claims includes both one and more than one such excipient.

A pharmaceutical compositions of the disclosure are formulated to be compatible with its intended route of administration. Examples of routes of administration include parenteral, e.g., intravenous, intradermal, subcutaneous, oral (e.g., inhalation), transdermal (topical), and transmucosal administration.

The present disclosure also provides the following biological test methods:

Test Method 1. GP2D Cell p-ERK Inhibition Test

### 1. Purpose

By HTRF, compounds that effectively inhibited p-ERK in ^{G12D}D mutant GP2D cells were screened.

### 2. Experimental Process

1). GP2D cells were seeded in a transparent 96-well cell culture plate, with 80 μL of cell suspension per well, containing 8,000 cells per well. The cell plate was placed in a carbon dioxide incubator and incubated overnight at 37° C.;
2). Take 2 μL of compound and add 78 μL of cell culture medium, mix well, take 20 μL of compound solution and add it to the corresponding cell plate wells, and place the cell plate back in the carbon dioxide incubator for continued incubation for 1 hours,
3). After the end of incubation, discard the cell supernatant and add 50 μL of 1× cell lysate to each well, and incubate at room temperature with shaking for 30 minutes,
4). Use detection buffer to dilute Phospho-ERK1/2 Eu Cryptate antibody and Phospho-ERK 1/2 d2 antibody by 20 times;
5). Take 16 μL of cell lysate supernatant into a new 384 white microplate, add 2 μL of Phospho-ERK 1/2 Eu Cryptate antibody diluent and 2 μL of Phospho-ERK 1/2 d2 antibody diluent, and incubate at room temperature for at least 4 hours;
6). After the end of incubation, use a multi-label analyzer to read HTRF excitation: 320 nm, emission: 615 nm, 665 nm;
7). Calculate the compound to be tested IC₅₀.
   Test Method 2. AGS Cell p-ERK Inhibition Test

### 1. Purpose

By HTRF, compounds that effectively inhibit p-ERK in KRAS^{G12D} mutant AGS cells were screened.

### 2. Experimental Process

1). AGS cells were seeded in a transparent 96-well cell culture plate, with 80 μL of cell suspension per well, containing 10,000 cells per well. The cell plate was placed in a carbon dioxide incubator and incubated overnight at 37° C.;
2). At the end of incubation, discard the cell supernatant, add 80 μL of culture medium per well, the culture medium contains 0.02% serum, place the cell plate in a carbon dioxide incubator, and incubate overnight at 37° C.;
3). Take 2 μL of compound and add 78 μL of cell culture medium, mix well, take 20 μL of compound solution and add it to the corresponding cell plate wells, and place the cell plate back in the carbon dioxide incubator for continued incubation for 3 hours;
4). After the end of incubation, discard the cell supernatant and add 50 μL of 1× cell lysate to each well, and incubate at room temperature with shaking for 30 minutes;
5). Use detection buffer to dilute Phospho-ERK1/2 Eu Cryptate antibody and Phospho-ERK 1/2 d2 antibody by 20 times;
6). Take 16 μL of cell lysate supernatant into a new 384 white microplate, add 2 μL of Phospho-ERK1/2 Eu Cryptate antibody diluent and 2 μL of Phospho-ERK 1/2 d2 antibody diluent, and incubate at room temperature for at least 4 hours;
7). After the end of incubation, use a multi-label analyzer to read HTRF excitation: 320 nm, emission: 615 nm, 665 nm;
8). Calculate the compound to be tested IC₅₀.

### Test Method 3. Anti-Cell Proliferation of Compounds in Tumor Cell Line AsPC-1

### Study Objectives

In this experiment, the effect of compound on cell proliferation was investigated by detecting the effect of compound on in vitro cell activity in KRAS^{G12D} mutant tumor cell line AsPC-1.

### Experimental Materials

Cell line: AsPC-1, tumor type: Pancreatic cancer; Growth characteristics: Adherent growth; culture
method: RPMI 1640+10% FBS
Ultra Low Cluster-96 well plate (Corning-7007)
Greiner CELLSTAR 96-well plate (#655090)
Promega CellTiter-Glo 3D Luminescence Cell Viability Assay Kit (Promega-G9683)
2104-10 En Vision Plate Reader, PerkinElmer
RPMI 1640, DMEM, PBS (phosphate buffer), FBS (fetal bovine serum), antibiotic-antimycotic (antibiotic-antifungal), L-glutamine (L-glutamine), DMSO (dimethyl sulfoxide)

### Experimental Methods and Procedures

### Cell Culture

Tumor cell lines were incubated in an incubator at 37° C., 5% CO₂ under the culture conditions indicated by the culture method. Periodic passages are performed, and cells in logarithmic growth are taken for plating.

### Cell Plating

Cells were stained with Trypan Lang and live cells were counted.

Adjust the cell concentration to the appropriate concentration.

### Cell Line: AsPC-1; Density (Per Well): 7000 Cells.

Add 135 μL of cell suspension to each well of the ULA plate, and add the same volume of cell-free medium to the vehicle control air.

After plating, immediately centrifuge the ULA plate at room temperature for 10 minutes and centrifuge at 1000 rpm. Note: After centrifugation, care must be taken not to cause unnecessary shaking.

Incubate plates overnight in an incubator at 37° C., 5% CO₂, and 100% relative humidity.

### Preparation of 10× Compound Working Solution and Compound Treatment Cells (Day 1)

After preparing the 10× compound working solution (DMSO 10× working solution), add 15 μl of 10× compound working solution to the ULA culture plate, and add 15 μL of DMSO-cell culture mixture to the vehicle control and blank control respectively.

The 96-well cell plates were returned to the incubator for incubation for 120 hours.

Cell spheroids were observed daily until the end of the experiment.

### CellTiter-Glo Luminescence Cell Viability Assay (Day 5)

The following steps were performed in accordance with the instructions for the Promega Cell Titer-Glo 3D Luminescence Cell Viability Assay Kit (Promega #G9683).

Add 150 μL of CellTiter-Glo 3D reagent (equal to the volume of cell culture medium in each well) to each well. Wrap the cell plate with aluminum foil to protect from light.

Shake the plate on an orbital shaker for 5 minutes.

Carefully pipet up and down 10 times and mix well the mixture. Ensure that the multicellular spheroids are adequately separated before proceeding to the next step.

The solution in the ULA plate was then transferred to a black bottom plate (#655090) and left at room temperature for 25 minutes to stabilize the luminescent signal.

Luminescent signal is detected on the 2104 En Vision reader.

### Data Analysis

The inhibition rate (IR) of the test compound was calculated by using the following formula: IR (%)=(1−(RLU compound−RLU vehicle control)/(RLU vehicle control−RLU vehicle control))*100%. Inhibition rates were calculated for different concentrations of compounds in Excel, and then plotted with GraphPad Prism software and relevant parameters were calculated, including minimum inhibition, maximum inhibition, and IC₅₀.

Test method 4. Pharmacokinetic Study on Oral and Intravenous Test Compounds in CD-1 Mice

### Experimental Purpose

Test the in vivo pharmacokinetics of oral and intravenous compounds in CD-1 mice.

### Protocol

The test compound was mixed with 5% DMSO+95 centrifugal 10% HP-β-CD) aqueous solution, vortexed and sonicated to prepare a 0.5 mg/mL clear solution (intravenous) or a 3 mg/mL clear solution (oral), and the microporous filter membrane was filtered for later use. Male CD-1 mice aged 7 to 10 weeks were selected and intravenously injected with the candidate compound solution. The candidate compound solution was administered orally. Whole blood was collected for a certain period of time, plasma was prepared, drug concentration was analyzed by using the LC-MS/MS method, and pharmacokinetic parameters were calculated by using Phoenix WinNonlin software (Pharsight, USA).

### Technical Effect

The compound of the present invention has good cell proliferation inhibitory activity on KRAS (12D mutant cells and has significant inhibitory effect on p-ERK of KRASG12D mutant cells.

### EXAMPLES

The disclosure is further illustrated by the following examples and synthesis schemes, which are not to be construed as limiting the disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure and/or scope of the appended claims.

### Example 1

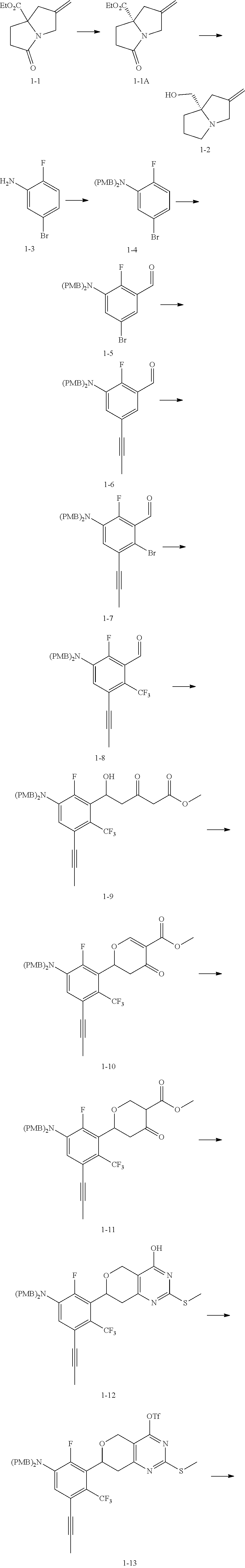

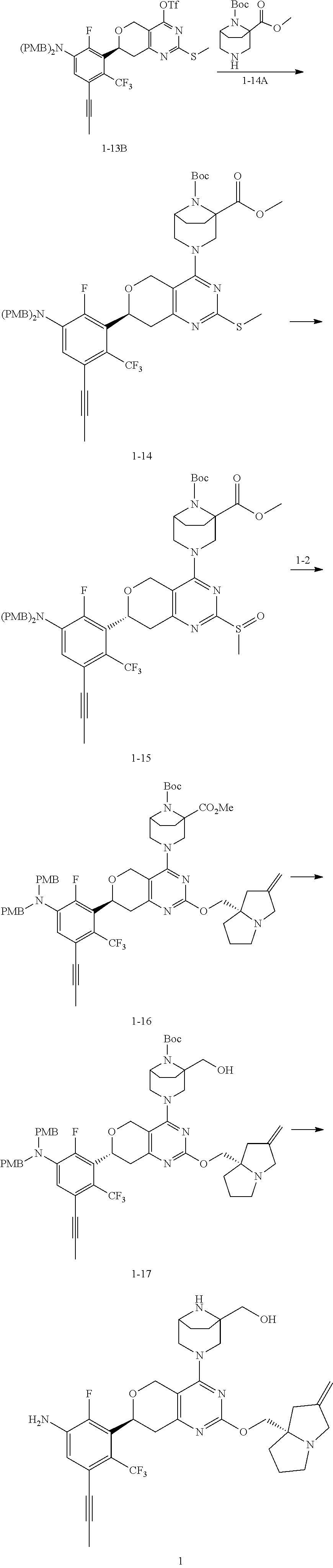

### Step 1

Preparation of supercritical liquid chromatography (SFC) (chromatographic column: ChiralPak IH, 250*50 mm, 10 um; mobile phase: A: Supercritical carbon dioxide, B: [0.1% ammonia water-ethanol]; B %: 20%-20% running time 3.7 min) to obtain compound 1-1A, SFC analytical method (column: Chiralpak IH-3, 100×4.6 mm I.D., 3 um; mobile phase: A (supracritical carbon dioxide) and B (ethanol, containing 0.1% isopropylamine); gradient: B %=10˜50%, 4 min; flow rate: 3.4 mL/min; wavelength: 220 nm; pressure: 2000 psi), compound 1-1A Rt=1.489 min, ee value 98.82%. 1H NMR (400 MHz, CDCl₃) δ=4.99-4.86 (m, 2H), 4.26-3.95 (m, 3H), 3.59 (m, 1H), 3.00-2.88 (m, 1H), 2.87-2.12 (m, 4H), 1.91 (s, 1H), 1.20-1.08 (m, 3H).

### Step 2

Dissolve lithium tetrahydroaluminum (1.55 g, 40.15 mmol) in anhydrous tetrahydrofuran (30 mL), cool to 0° C., add anhydrous tetrahydrofuran (20 mL) solution of compound 1-1A (2.8 g, 13.38 mmol) under nitrogen protection, and react at 70° C. for 1 hour. Add 1.5 mL of water to the reaction solution at 0° C., add 1.5 mL of 15% NaOH solution, add 4.5 mL of water, stir for 20 minutes, filter the reaction solution, wash the filter cake with 10 mL of tetrahydrofuran, and concentrate the filtrate to obtain compound 1-2. ¹H NMR (400 MHZ, CDCl₃) δ=4.99-4.86 (m, 2H), 4.26-3.95 (m, 3H), 3.59 (m, 1H), 3.00-2.88 (m, 1H), 2.74-2.27 (m, 4H), 1.91 (s, 1H), 1.20-1.08 (m, 3H).

### Step 3

Compounds 1-3 (480 g, 2.53 mol) were weighed, DMF (2,500 mL) was added, 4-methoxychlorobenzyl (5.18 mol, 702.79 mL) was added, potassium carbonate (872.82 g, 6.32 mol was added, potassium iodide (419.35 g. 2.53 mol) was reacted at 65° C. for 2 hours. Add water (1,000 mL), extract with ethyl acetate (1,000 mL*3), and concentrate the organic phase under reduced pressure to obtain compound 1-4, MS m/z=430.0 [M+H]⁺.

### Step 4

Weigh compound 2,2,6,6-tetramethylpiperidine (220.59 g, 1.56 mol, 265.13 mL), THF (3000 mL) was added, n-butyllithium (2.5 M, 499.73 mL) was added at −5° C., stirred for 0.5 hours, and cooled to −60° C., compound 1-4280 g, 624.67 mmol was added for 0.5 hours (DMF228.28 g, 3.12 mol, 240.30 mL) was added. The reaction was continued for 0.5 hour. Pour the reaction solution into water (1,000 mL), add 1N hydrochloric acid to adjust the pH to 7, extract with ethyl acetate (1,000 mL*3), concentrate the organic phase under reduced pressure, and separate by column chromatography (petroleum ether: Ethyl acetate=10:1) to obtain compounds 1-5.

### Step 5

Weigh Compounds 1-5 (370 g, 807.30 mmol), toluene (1500 mL), dichlorobis [di-tert-butyl-(4-dimethylaminophenyl)phosphine]palladium (2.86 g, 4.04 mmol, 2.86 mL) and add tributyl (1-propynyl) tin (265.69 g, 807.30 mmol), and reacted at 120° C. under nitrogen protection for 2 hours. The reaction solution was concentrated under reduced pressure and separated by column chromatography (petroleum ether: Ethyl acetate=5:1) to obtain compounds 1-6. MS m/z=418.1 [M+H]⁺.

### Step 6

Compounds 1-6 (450 g, 970.13 mmol) were weighed, DMF (100 mL) was added, N-bromosuccinimide (189.93 g, 1.07 mol) was added, and the solution was reacted at 25° C. for 2 hours N-bromosuccinimide (17.27 g, 97.01 mmol) was added, and the reaction was continued for 3 hours. The reaction solution was concentrated under reduced pressure and separated by column chromatography (petroleum ether: Ethyl acetate=5:1) to obtain compounds 1-7. MS m/z=496.0 [M+H]⁺.

### Step 7

Compounds 1-7 (55 g, 110.81 mmol) were weighed, DMF (300 mL) was added, methyl fluorosulfonyl difluoroacetate (42.57 g, 221.61 mmol, 28.19 mL) was added, copper iodide (42.21 g, 221.61 mmol), and the reaction was carried out under nitrogen protection at 110° C. for 2 hours. Add 500 ml of water to quench, extract with ethyl acetate (600 mL*3), combine the extracted organic phases, wash with water (800 mL*2) and saturated brine (800 mL), dry with anhydrous sodium sulfate, filter, and concentrate. Column chromatography separation (petroleum ether: Ethyl acetate=10:1) to obtain compounds 1-8. MS m/z=485.9 [M+H]⁺.

### Step 8

For tetrahydrofuran solution (350 ml) of sodium hydrogen (6.34 g, 158.61 mmol, 60% purity), added methyl acetate (158.61 mmol, 17.10 mL) dropwise at 0° C. for 15 minutes. Cool to −20° C. and then add n-butyl lithium dropwise (2.5 M, 63.44 mL). After dropwise addition, continue stirring for 15 min, and then add compound 1-8 (35 g, 72.10 mmol) in tetrahydrofuran (350 mL). Reaction 0.5 hour. The reaction was quenched by adding 200 mL of saturated ammonium chloride solution, extracted with ethyl acetate (300 mL*2), combined with the extracted organic phase, washed with saturated brine (400 mL), dried with anhydrous sodium sulfate, filtered, and concentrated under reduced pressure. Column chromatography separation (petroleum ether: Ethyl acetate=10:1-1:1) to obtain compound 1-9. MS m/z=624.2 [M+Na]⁺.

### Step 9

Compounds 1-9 (38 g, 63.17 mmol) were weighed, dichloromethane (300 mL) was added, and N,N-dimethylformamide dimethylacetal (9.03 g, 75.80 mmol) was added. Reaction at 25° C. for 16 hours. Cool to 0° C., add boron trifluoride diethyl etherate (10.76 g, 75.80 mmol, 9.32 mL), continue stirring at 0° C. for 1 hour, add 200 mL of saturated sodium bicarbonate solution to the system, separate the organic phase, extract the aqueous phase with 200 mL of dichloromethane, combine the extracted organic phase, wash with 250 mL of saturated brine, dry with anhydrous sodium sulfate, filter, and concentrate under reduced pressure. Column chromatography separation (petroleum ether: Ethyl acetate=10:1-1:1) to obtain compound 1-10. MS m/z=612.1 [M+H]⁺.

### Step 10

Compound 1-10 (30 g, 49.05 mmol) was weighed, tetrahydrofuran (300 mL) was added, and lithium tris sec-butylborohydride (1 M, 53.96 mL) was added at −60° C. React at −60° C. for 1 hour, add 200 mL of water to the system to quench the reaction, extract with ethyl acetate (300 mL*2), combine the extracted organic phases, wash with 300 mL of saturated brine, dry with anhydrous sodium sulfate, filter, and concentrate under reduced pressure. Column chromatography separation (petroleum ether: Ethyl acetate=10:1-5:1) to obtain compound 1-11. MS m/z=614.1 [M+H]⁺.

### Step 11

Compounds 1-11 (20 g, 32.59 mmol) were weighed, ethanol (200 mL) was added, and 2-methyl-2-thioisourea sulfate (27.22 g. 97.78 mmol) was added, sodium carbonate (6.91 g, 65.19 mmol) was reacted at 50° C. for 13 hours. The reaction solution was concentrated and dried, 40 mL of water was added, extracted with ethyl acetate (50 mL*2), the extracted organic phase was combined, washed with 60 mL of saturated brine, and dried with anhydrous sodium sulfate. The compounds 1-12 were concentrated under reduced pressure by filtration. MS m/z=654.3 [M+H]⁺.

### Step 12

Compounds 1-12 (21 g, 32.13 mmol), DMF (200 mL), N,N-diisopropylethylamine (12.46 g, 96.38 mmol, 16.79 mL), N,N-bis(trifluoromethylsulfonyl) aniline (13.77 g, 38.55 mmol), and reacted at 25° C. for 1 hour. Added with 300 mL of water to the system, extracted with ethyl acetate (300 mL*3), washed with water (400 mL*2) and saturated brine (400 mL), dried with anhydrous sodium sulfate, filtered, and concentrated under reduced pressure. Column chromatography separation (petroleum ether: Ethyl acetate=10:1) to obtain compounds 1-13. Preparation SFC splitting was performed (chromatographic column: DAICEL CHIRALPAK IG (250 mm*50 mm, 10 μm); mobile phase: [supercritical carbon dioxide-ethanol (0.1% ammonia)]; ethanol (0.1% ammonia water) %: 25%-25%) to obtain compound 1-13B. Chiral SFC analysis (chromatographic column: ChiralPak IG-3 (100 mm*4.6 mm, 3 μm); mobile phase: [supercritical carbon dioxide-ethanol (0.05% diethylamine)]; (ethanol (0.05% diethylamine) %) %: 5%-40%), Rt=3.055 minutes for compound 1-13B, ee value 99%.

### Step 13

Compound 1-13B (200 mg, 254.53 μmol) and compound 1-14A (174.74 mg) were added to N, N-dimethylformamide (2 mL), and then N, N-diisopropylethylamine (131.58 mg, 1.02 mmol) was added. The resulting reaction solution was heated to 105° C. under nitrogen protection and stirred for 1 hour. The reaction solution was concentrated under reduced pressure to obtain crude product. Crude product column chromatography purification (petroleum ether. Ethyl acetate=5:1) to obtain compounds 1-14. MS m/z=906.3 [M+H]⁺.

### Step 14

Compounds 1-14 (120 mg, 132.45 μmol) were dissolved in methylene chloride (2 mL), then metachloroperoxybenzoic acid (29.58 mg, 145.70 μmol, 85% purity) was added, and the reaction solution was stirred and reacted for 2 hours at 20° C. under nitrogen protection. The reaction solution was diluted with 30 mL dichloromethane, then washed with 5 mL saturated sodium bicarbonate solution and 5 mL saturated brine, the organic phase dried, and concentrated under reduced pressure to obtain compounds 1-15. MS m/z=922.7 [M+H]⁺.

### Step 15

Sodium tert-butanol (23.97 mg, 249.46 μmol) and compound 1-2 (38.22 mg, 249.46 μmol) were added to tetrahydrofuran (1.5 mL), and the reaction was stirred at 20° C. for 0.5 hours. Then, a solution of compound 1-15 (115 mg, 124.73 μmol) in tetrahydrofuran (1 mL) was added to the above system, and the reaction was continued to be stirred for 1 hour. Adjust the pH of the reaction solution to 7 by using 0.5M hydrochloric acid. Then, add 20 mL of ethyl acetate and 10 ml of water, and stir to dissolve. Separate the aqueous layer. Wash the organic phase with 2 mL of saturated saline solution. Dry the organic phase with anhydrous sodium sulfate, filter, and concentrate the filtrate under reduced pressure to obtain compound 1-16. MS m/z=1011.5 [M+H]⁺.

### Step 16

Compounds 1-16 (120.00 mg, 118.68 μmol) were dissolved in tetrahydrofuran (2 mL), the resulting solution was cooled to 0° C., and then lithium tetrahydroaluminum (1 M, 118.68 μL) was added dropwise to the mixture, and the reaction was stirred for 0.5 hours. The reaction solution was carefully quenched with 0.2 mL of water, then 0.5 g of anhydrous sodium sulfate was added and stirred for 2 minutes. Diatomaceous earth was paddled and filtered. The filter cake was rinsed with 20 mL of tetrahydrofuran, the filtrate was collected, and concentrated under reduced pressure to obtain compound 1-17. MS (ESI) m/z=983.4 [M+H]⁺.

### Step 17

Compounds 1-17 (120 mg, 122.06 μmol) were added to trifluoroacetic acid (2 mL), and the reaction was stirred and reacted at 20° C. for 2 hours. The reaction solution was concentrated under reduced pressure to obtain crude product. The crude product was separated by high performance liquid chromatography (chromatographic column: Xtimate C18 150*40 mm*5 μm; mobile phase: [water (0.05% hydrochloric acid)-acetonitrile]; acetonitrile): 10%-40%, concentrated under reduced pressure to obtain the hydrochloride salt of compound 1. MS m/z=643.3 [M+H]⁺.

### Example 2

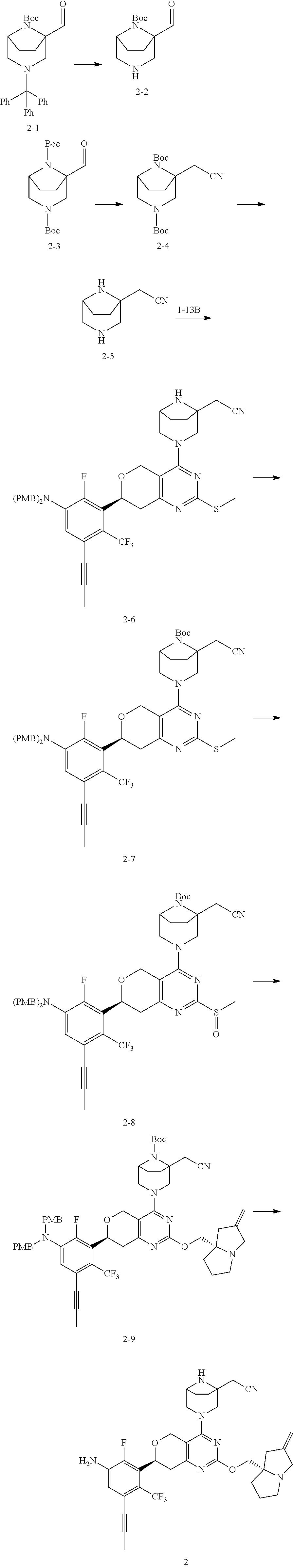

### Step 1

Compound 2-1 (2.3 g, 4.77 mmol) was dissolved in dioxane (30 mL), and then hydrochloric acid (1 M, 14.30 mL) was added. The reaction solution was stirred and reacted for 1 hour under nitrogen protection at 20° C. The reaction solution was concentrated under reduced pressure. 20 ml of water and 50 mL of ethyl acetate were added to the residue and stirred to make it fully dissolved. The aqueous phase was separated. The organic phase was washed with 10 mL of 0.5 M hydrochloric acid. The aqueous phase was combined, pH=10 was adjusted with 20% sodium carbonate solution, then extracted with dichloromethane (20 mL×2), the organic phase was combined, and concentrated under reduced pressure to obtain compound 2-2.

### Step 2

For the compound 2-2 (1.2 g, 4.77 mmol), it was added 20 mL of dichloromethane, then Boc₂O (2.08 g, 9.53 mmol) was added, and the reaction was stirred at 20° C. for 1 hour. The reaction solution was concentrated under reduced pressure to obtain crude product. Crude product column chromatography purification (petroleum ether: Ethyl acetate=5:1) obtained compound 2-3. ¹HNMR (400 MHZ, CDCl₃) δ:9.55-9.47 (m, 1H), 4.40-4.00 (m, 2H), 3.95-3.55 (m, 1H), 3.30-2.90 (m, 2H), 2.25-1.91 (m, 2H), 1.78 (s, 2H), 1.47 (s, 18H).

### Step 3

Potassium tert-butanol (830.68 mg, 7.40 mmol) was added to glycol dimethyl ether (25 mL), the resulting mixture was cooled to −78° C., then added p-methylbenzene sulfonyl methyl isonitrile (794.91 mg, 4.07 mmol), after addition, stirred for 30 minutes, and then added compound 2-3 (1.26 g, 3.70 mmol) dropwise to the mixture (25 mL), after dropping, stirred for 30 minutes, removed from the cooling bath, increased to room temperature for 20 minutes, finally added methanol 40 mL, and the resulting final reaction solution was heated to 90° C. for 1 hour. The reaction solution was concentrated under reduced pressure to obtain crude product. Crude product column chromatography purification (petroleum ether: Ethyl acetate=3:1) obtained compound 2-4. ¹HNMR (400 MHZ, CDCl₃) δ:4.22 (s, 1H), 3.65-3.55 (m, 2H), 3.30-2.62 (m, 4H), 1.99-1.89 (m, 2H), 1.65-1.30 (m, 20H).

### Step 4

Compound 2-4 (375 mg, 1.07 mmol) was dissolved in dichloromethane (2 mL), and then trifluoroacetic acid (1.22 g, 10.67 mmol) was added. The resulting reaction solution was stirred and reacted for 1 hour at 20° C. under nitrogen protection. The reaction solution was concentrated under reduced pressure, 20 mL of ethyl acetate was added to the residue, and then 1 mL of hydrogen chloride/ethyl acetate solution (4M) was added to it, and concentrated under reduced pressure to obtain the hydrochloride salt of compound 2-5.

### Step 5

Compound 1-13B (350 mg, 445.44 μmol) and the hydrochloride salt of compound 2-5 (299.51 mg) were added to N,N-dimethylformamide (3 mL), and then N,N-diisopropylethylamine (460.56 mg, 3.56 mmol) was added. The resulting reaction solution was heated to 105° C. under nitrogen protection and stirred for 1 hour. The reaction solution was concentrated under reduced pressure to give the crude product, which was dissolved in 50 mL of ethyl acetate, then washed with saturated brine (10 mL×2), dried in an organic phase, and concentrated under reduced pressure to obtain compounds 2-6. MS m/z=787.2 [M+H]⁺.

### Step 6

Compound 2-6 (368 mg, 467.67 μmol) and N,N-diisopropylethylamine (120.89 mg, 935.34 μmol) were added to dichloromethane (3 mL), and then Boc₂O (153.10 mg, 701.51 μmol) was added, and the reaction solution was stirred for 3 hours under nitrogen protection at 20° C. The reaction solution was concentrated under reduced pressure to obtain crude product. Crude product column chromatography purification (petroleum ether: Ethyl acetate=3:1) obtained compound 2-7. MS m/z=887.4 [M+H]⁺.

### Step 7

Compound 2-7 (220 mg, 248.03 μmol) was dissolved in methylene chloride (2 mL), and then metachloroperoxybenzoic acid (50.35 mg, 248.03 μmol, 85% purity) was added. The reaction solution was obtained under nitrogen protection and stirred and reacted at 20° C. for 2 hours. The reaction solution was concentrated under reduced pressure to obtain compounds 2-8. MS m/z=903.6 [M+H]⁺.

### Step 8

Compound 1-2 (75.68 mg, 493.91 μmol) was dissolved in THF (2 mL), then sodium tert-butanol (47.47 mg, 493.91 μmol) was added, and the reaction was stirred at 20° C. for 1 hour under nitrogen protection, and then a solution of compound 2-8 (223 mg, 246.96 μmol) in tetrahydrofuran (1 mL) was added to the solution, and after addition, the reaction was stirred at 20° C. for 0.5 hours. The reaction solution was dissolved in 10 mL of ethyl acetate, then washed with 5 mL of saturated brine, the organic phase dried, then filtered, and the filtrate was concentrated under reduced pressure to obtain compounds 2-9. MS m/z=992.5 [M+H]⁺.

### Step 9

Trifluoroacetic acid (2 mL) was added to compounds 2-9 (214 mg, 215.70 μmol), and the resulting reaction solution was stirred at 20° C. for 1 hour. The reaction solution was concentrated under reduced pressure to obtain crude product. The crude product was prepared by high performance liquid chromatography separation (chromatographic column: Xtimate C18 150*40 mm*5 μm; mobile phase: [water (0.05% hydrochloric acid)-acetonitrile]; acetonitrile): 10%-40%), concentrated under reduced pressure to obtain the hydrochloride salt of compound 2. MS m/z=652.2 [M+H]⁺.

### Example 3

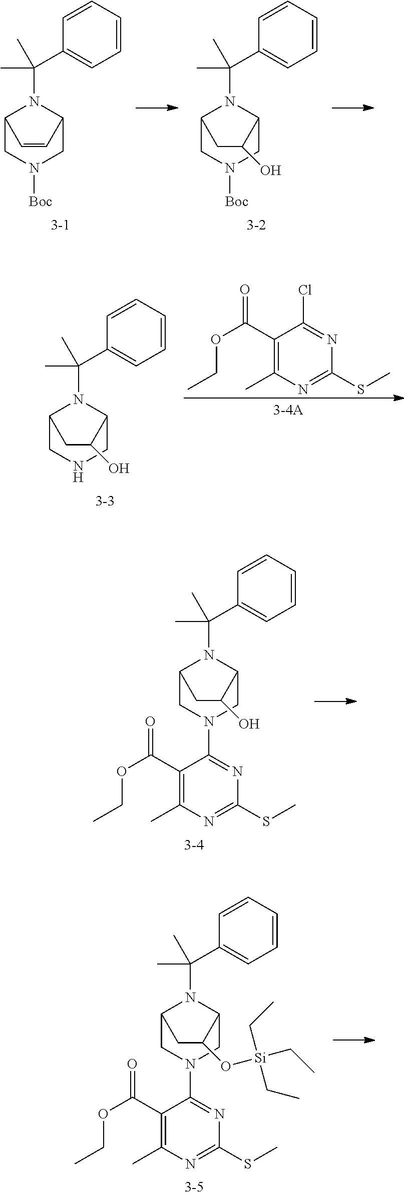

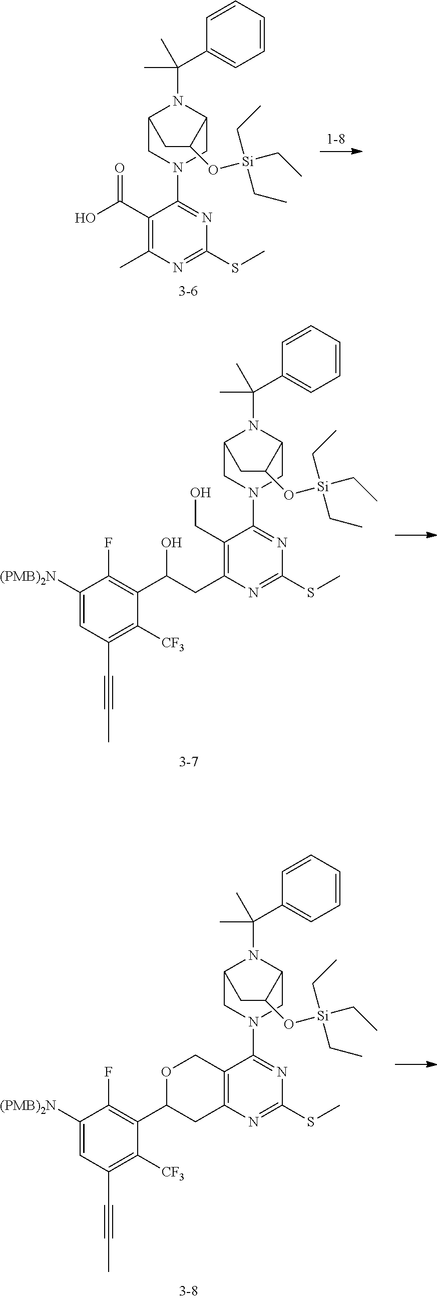

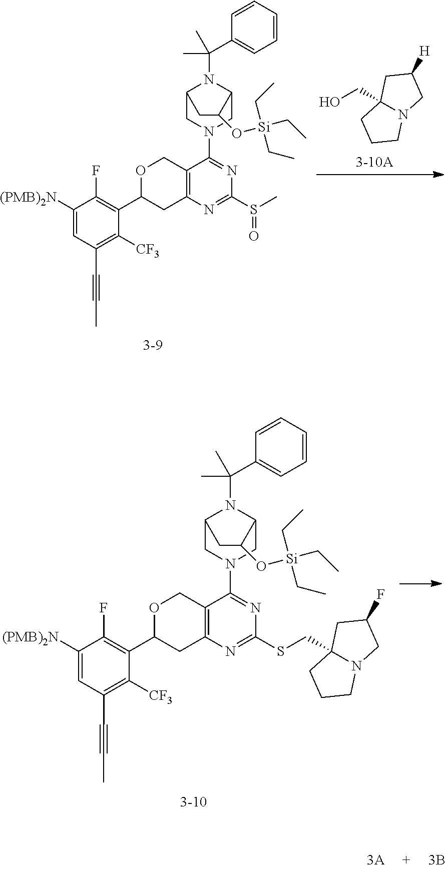

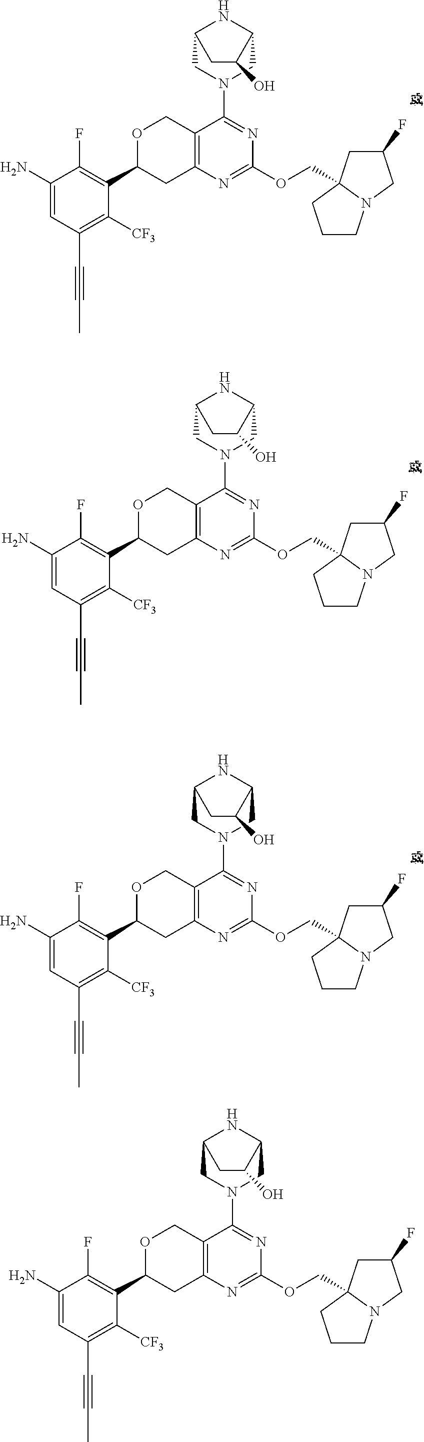 [SMILES: CC#Cc1cc(N)c(F)c([C@@H]2Cc3nc(OC[C@@]45CCCN4C[C@H](F)C5)nc(N4C[C@H]5C[C@H](O)[C@@H](C4)N5)c3CO2)c1C(F)(F)F ; CC#Cc1cc(N)c(F)c([C@@H]2Cc3nc(OC[C@@]45CCCN4C[C@H](F)C5)nc(N4C[C@H]5C[C@@H](O)[C@@H](C4)N5)c3CO2)c1C(F)(F)F ; CC#Cc1cc(N)c(F)c([C@@H]2Cc3nc(OC[C@@]45CCCN4C[C@H](F)C5)nc(N4C[C@@H]5C[C@H](O)[C@H](C4)N5)c3CO2)c1C(F)(F)F ; CC#Cc1cc(N)c(F)c([C@@H]2Cc3nc(OC[C@@]45CCCN4C[C@H](F)C5)nc(N4C[C@@H]5C[C@@H](O)[C@H](C4)N5)c3CO2)c1C(F)(F)F]

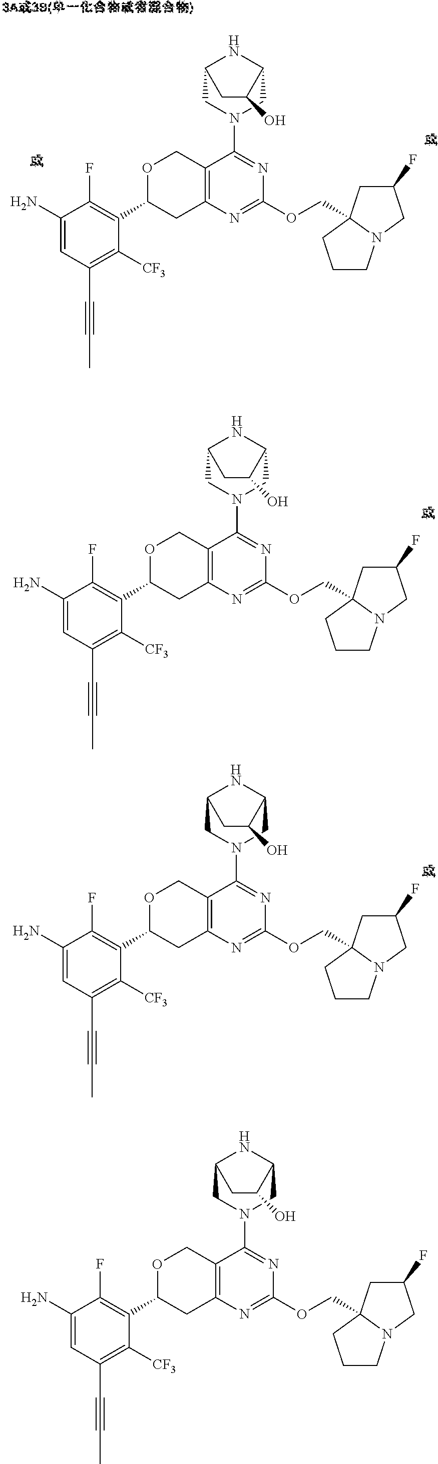 [SMILES: CC#Cc1cc(N)c(F)c([C@H]2Cc3nc(OC[C@@]45CCCN4C[C@H](F)C5)nc(N4C[C@H]5C[C@H](O)[C@@H](C4)N5)c3CO2)c1C(F)(F)F ; CC#Cc1cc(N)c(F)c([C@H]2Cc3nc(OC[C@@]45CCCN4C[C@H](F)C5)nc(N4C[C@H]5C[C@@H](O)[C@@H](C4)N5)c3CO2)c1C(F)(F)F ; CC#Cc1cc(N)c(F)c([C@H]2Cc3nc(OC[C@@]45CCCN4C[C@H](F)C5)nc(N4C[C@@H]5C[C@H](O)[C@H](C4)N5)c3CO2)c1C(F)(F)F ; CC#Cc1cc(N)c(F)c([C@H]2Cc3nc(OC[C@@]45CCCN4C[C@H](F)C5)nc(N4C[C@@H]5C[C@@H](O)[C@H](C4)N5)c3CO2)c1C(F)(F)F]

### Step 1

Borane tetrahydrofuran solution (1 M, 21 mL) was added slowly dropwise to anhydrous THF (20 mL) solution of compound 3-1 (1.7 g, 5.18 mmol) under nitrogen protection at 0° C., and then stirred at 25° C. for 12 hours. 5% NaOH solution (26.31 mmol, 21 mL) was added dropwise at 0° C., followed by di-oxygen water (4.88 g, 43.04 mmol, 4.14 mL, 30% purity) was added dropwise. React at 25° C. for 2 hours. The solution was quenched by slowly adding 50 mL of saturated sodium sulphite solution to the reaction solution and extracted with 50 mL of ethyl acetate. The organic phase was washed with 50 mL of salt, dried with anhydrous sodium sulfate, filtered, and the filtrate was concentrated under reduced pressure to obtain the crude product. The crude product was chromatographically separated with a thin layer of preparation (petroleum ether: Ethyl acetate=3:1) to obtain compound 3-2. MS m/z=347.2 [M+H]⁺.

### Step 2

To anhydrous dioxane (10 mL) solution of compound 3-2 (1 g, 2.89 mmol), it was added hydrochloric acid/dioxane solution (4 M, 10 mL), and the solution was reacted at 20 degrees for 0.5 hours. The reaction solution was concentrated under reduced pressure to obtain the hydrochloride salt of compound 3-3.

### Step 3

Compound 3-4A (750 mg, 3.04 mmol) was dissolved in anhydrous DCM (10 mL), followed by addition of triethylamine (584 mg, 5.77 mmol) and hydrochloride of compound 3-3 (921 mg) and reacted at 20° C. for 1 hour. A dichloromethane solution of compound 3-4 was obtained and directly injected into the next step reaction. MS m/z=457.2 [M+1]⁺.

### Step 4

For dichloromethane solution (10 mL) of compound 3-4 obtained in step 3, it was added with triethylchlorosilane (871 mg, 5.78 mmol) and imidazole (590 mg, 8.67 mmol), respectively. React d at 25° C. for 12 hours. The reaction solution was diluted with 10 mL of water and extracted with 10 mL of dichloromethane. The organic phase was washed with 10 mL of salt, dried with anhydrous sodium sulfate, filtered, and the filtrate was concentrated under reduced pressure to obtain the crude product. The crude product was separated by column chromatography (petroleum ether: Ethyl acetate=50:1) to obtain compound 3-5. MS m/z=$71.3 [M+1]⁺.

### Step 5

THF (20 mL) solution of compound 3-5 (1.43 g, 2.50 mmol), and lithium hydride tetrahydrofuran solution (2.5 M, 2.00 mL) was slowly added at 0° C. The solution was reacted at 25° C. for 5 hours, and ethyl acetate (10 mL) was added dropwise to the reaction solution to quench the reaction, and the resulting suspension was filtered, and the filtrate was concentrated under reduced pressure to obtain the crude product. The crude product was purified by column chromatography (petroleum ether: ethyl acetate=20:1) to obtain compound 3-6. MS m/z=529.3 [M+1]⁺.

### Step 6

Compound tetramethylpiperidine (460 mg, 3.26 mmol) was dissolved in anhydrous tetrahydrofuran (1.0 mL), cooled to −40° C. under nitrogen protection, and then n-butyllithium (2.5 M 1.3 mL) was added dropwise thereto. The reaction was stirred for 0.5 hours, compound 3-6 (430 mg, 813 μmol) was dissolved in anhydrous tetrahydrofuran (0.5 mL), and added dropwise to the reaction vessel at −60° C. After dripping, the reaction was stirred for 0.5 hours. Finally, an anhydrous tetrahydrofuran 0.5 mL solution of compound 1-8 (486 mg, 1.00 mmol) was added to the reaction under −60° C. and then heated to 20° C. for 2.5 hours. Add 20 ml of water to quench reaction, and then extract with ethyl acetate (20 mL). The organic phase was washed with 20 mL of salt, dried with anhydrous sodium sulfate, filtered, and the filtrate was concentrated under reduced pressure to obtain the crude product. The crude product was purified by column chromatography (petroleum ether:ethyl acetate=5.1), and then a thin layer of chromatographic separation (petroleum ether: Ethyl acetate=3:1) obtained compound 3-7. MS m/z=1014.5 [M+H]⁺.

### Step 7

Compounds 3-7 (70 mg, 69.0 μmol) were dissolved in anhydrous toluene (1.5 mL), cyanomethylenetrin-butylphosphine (150 mg, 621.50 μmol) was added, and the reaction was stirred at 110° C. for 12 hours after nitrogen replacement. The reaction solution was cooled and concentrated under reduced pressure to obtain the crude product. Preparation thin layer chromatography purification of crude product (petroleum ether: Ethyl acetate=5:1) obtained compounds 3-8. MS m/z=996.5 [M+H]⁺.

### Step 8

Compound 3-8 (22.5 mg, 22.58 μmol) was dissolved in anhydrous dichloromethane (0.5 mL), 5 mg, 24.63 μmol, 85% purity) was added, and the reaction was stirred at 25° C. for 12 hours. Add 4 mL of saturated sodium sulfite solution to the reaction solution to quench the reaction and extract with dichloromethane (20 mL*3). The organic phase was washed with 20 mL of brine, dried with anhydrous sodium sulfate, filtered, and the filtrate was concentrated under reduced pressure to obtain the crude product. Preparation thin layer chromatography purification of crude product (petroleum ether: Ethyl acetate=5:1) obtained compounds 3-9. MS m/z=1012.6 [M+H]⁺.

### Step 9

Compound 3-9 (19 mg, 18.8 μmol) was dissolved in anhydrous toluene (0.5 mL), sodium tert-butoxide (7.22 mg. 75.1 μmol), 4 Å molecular sieve (10 mg) and compound 3-10A (12 mg, 75.08 μmol) were added, and the solution was stirred at 100° C. for 12 hours. The reaction solution was cooled, filtered, and the filtrate was concentrated under reduced pressure to obtain the crude product. Crude product preparation thin layer chromatographic purification (ethyl acetate:methanol=10:1) to obtain compound 3-10. MS m/z=1107.7 [M+H]⁺.

### Step 10

Compounds 3-10 (17 mg, 15.35 μmol) were dissolved in trifluoroacetic acid (5 mL) and reacted at 20° C. for 12 hours. The reaction solution was concentrated under reduced pressure to obtain the crude product. The crude product was prepared for high performance liquid chromatography separation (column: Phenomenex luna C18 150*25 mm*10 μm; mobile phase: [water (0.075% trifluoroacetic acid)-acetonitrile]; acetonitrile: 18%-48% to obtain the trifluoroacetate salt of compound 3A and the trifluoroacetate salt of 3B.

Trifluoroacetate of compound 3A (single compound or mixture), MS m/z=635.3 [M+H]⁺. ¹H NMR (CD₃OD, 400 MHz) δ 6.94 (d, J=8.50 Hz, 1H), 5.71-5.50 (m, 1H), 5.17 (br dd, J=10.94, 4.06 Hz, 1H), 4.80 (br d, J=13.63 Hz, 2H), 4.62-4.48 (m, 3H), 4.17-3.73 (m, 7H), 3.54-3.36 (m, 3H), 3.31-3.25 (m, 1H), 2.93 (br dd, J=18.01, 3.75 Hz, 1H), 2.77-2.48 (m, 3H), 2.46-2.28 (m, 3H), 2.26-2.12 (m, 1H), 2.04 (s, 3H), 1.96 (dd, J=14.01, 4.13 Hz, 1H):

Trifluoroacetate of compound 3B (single compound or mixture), MS m/z=635.2 [M+H]⁺. 1H NMR (CD₃OD, 400 MHz) δ 6.93 (d, J=8.50 Hz, 1H), 5.69-5.49 (m, 1H), 5.30 (d, J=13.88 Hz, 1H), 5.18 (br dd, J=11.19, 3.56 Hz, 1H), 4.87-4.78 (m, 2H), 4.69-4.50 (m, 3H), 4.29 (br d, J=13.26 Hz, 1H), 4.20-3.87 (m, 6H), 3.63 (br d, J=14.51 Hz, 1H), 3.53-3.42 (m, 1H), 3.35 (br s, 1H), 2.93 (br dd, J=17.70, 3.56 Hz, 1H), 2.77-2.51 (m, 3H), 2.47-2.28 (m, 3H), 2.26-2.12 (m, 1H), 2.04 (s, 3H), 1.83 (dd, J=13.88, 4.25 Hz, 1H).

### Example 4

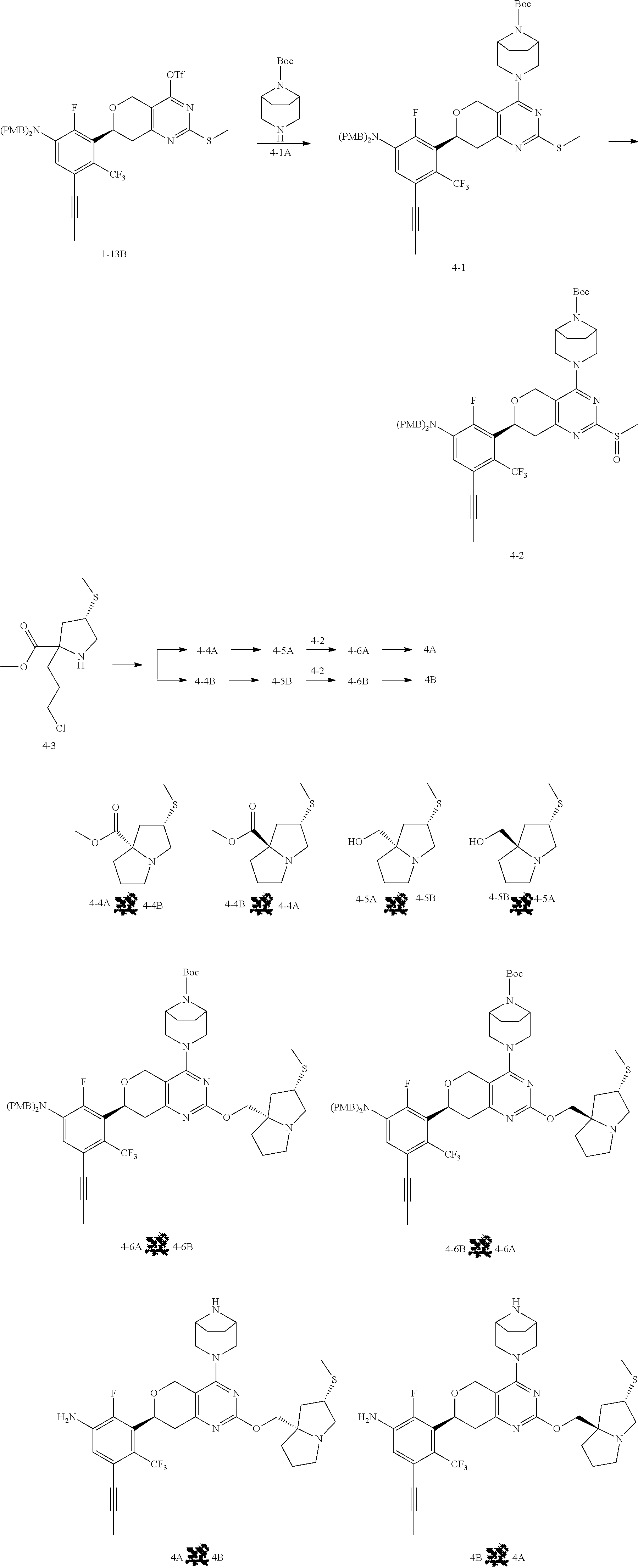

### Step 1

Compound 1-13B (240 mg, 305.44 μmol) and compound 4-1A (97.26 mg, 458.16 μmol) were dissolved in DMF (5 mL), DIPEA (916.33 μmol, 159.61 μL) was added, and then stirred at 100° C. for 1 hour. Extract with 30 mL of ethyl acetate, wash the organic phase with 50 mL of salt, dry with anhydrous sodium sulfate, filter, and concentrate the filtrate under reduced pressure to obtain the crude product. The crude product was chromatographically separated with a thin layer of preparation (petroleum ether: Ethyl acetate=5:1) to obtain compound 4-1. MS m/z=848.5 [M+H]⁺.

### Step 2

Compound 4-1 (200 mg, 235.86 μmol) was dissolved in dichloromethane (5 mL), metachloroperoxybenzoic acid (40.70 mg, 235.86 μmol, 85% purity) was added, and stirred at 25° C. for 1 hour. Add 5 mL of dichloromethane for extraction, wash with 10 mL of saturated sodium bicarbonate, dry with anhydrous sodium sulfate, filter, and concentrate the filtrate under reduced pressure to obtain the crude product. The crude product was chromatographically separated with a thin layer of preparation (dichloromethane:methanol=10:1) to give compound 4-2. MS m/z=864.3 [M+H]⁺.

### Step 3

Compounds 4-3 (610 mg, hydrochloride) were dissolved in acetonitrile (10 mL), to which potassium carbonate (1.46 g, 10.6 mmol) and potassium iodide (35.1 mg, 212 μmol) were added, and the reaction solution was reacted at 25° C. for 12 hours. The reaction solution was filtered, and the filtrate was concentrated under reduced pressure. The crude product was purified by column chromatography (petroleum ether:ethyl acetate=5:1), and then separated by a thin layer of chromatography (petroleum ether:ethyl acetate=1:1) to give compound 4-4A and compound 4-4B, wherein compound 4-4A (petroleum ether:ethyl acetate=1:1, R_{f}=0.21), MS m/z=216.0 [M+1]⁺, compound 4-4B (petroleum ether:ethyl acetate=1:1, R_{f}=0.12), MS m/z=216.1 [M+1]⁺.

Compound 4-4A: 1H NMR (400 MHZ, CDCl₃) δ ppm 3.75 (s, 3H), 3.54 (dd, J=6.5, 9.0 Hz, 1H), 3.33 (tt, J=6.4, 10.6 Hz, 1H), 3.13 (td, J=6.4, 10.8 Hz, 1H), 2.86-2.75 (m, 2H), 2.52 (t, J=9.6 Hz, 1H), 2.30-2.18 (m, 1H), 2.15 (s, 3H), 1.99-1.81 (m, 3H), 1.61 (dd, J=11.3, 12.6 Hz, 1H); Compound 4-4B: ¹H NMR (400 MHZ, CDCl₃) δ ppm 3.67 (s, 3H), 3.28-3.08 (m, 2H), 3.06-2.92 (m, 2H), 2.56 (td, J=7.4, 9.5 Hz, 1H), 2.37-2.17 (m, 2H), 2.09 (dd, J=6.9, 13.2 Hz, 1H), 2.04 (s, 3H), 1.79-1.69 (m, 2H), 1.67-1.56 (m, 1H).

### Step 4

Compound 4-4A (102 mg, 474 μmol) was dissolved in THF (5.0 mL), lithium tetrahydroaluminum (2.5 M, 0.3 mL) was added dropwise at 0° C., and the reaction solution was heated to 25° C. for 1 hour. Add 0.03 mL of water, 0.03 mL of 15% aqueous sodium hydroxide solution and 0.1 mL of water dropwise at 0° C., and stir for 0.5 hours. The mixture was filtered, the filter cake was washed with 10 ml of ethyl acetate, and the filtrate was concentrated under reduced pressure to give compound 4-5A, MS m/z=188.1 [M+1]⁺.

### Step 5

Compound 4-2 (120 mg, 139 μmol), compound 4-5A (89 mg, 475 μmol), 4 Å molecular sieve (120 mg) and sodium tert-butanol (80 mg, 832 μmol) were added to toluene (15 mL), and the temperature was increased to 100° C. for 6 hours. The reaction solution was filtered, the filter cake was washed with 10 mL of ethyl acetate, and the filtrate was concentrated under reduced pressure to obtain the crude product. The crude product was purified by preparing a thin layer of chromatographic separation (dichloromethane:methanol=10:1) to obtain compound 4-6A, MS m/z=987.4 [M+1]⁺.

### Step 6

Compound 4-6A (81 mg, 82.0 μmol) was dissolved in dichloromethane (10.0 mL), to which trifluoroacetic acid (3.07 g, 26.9 mmol, 2 mL) was added, and the reaction solution was reacted at 25° C. for 12 hours. The reaction solution was concentrated under reduced pressure to obtain the crude product. The crude product was prepared for high performance liquid chromatography separation (column: Phenomenex luna C18 150*25 mm*10 μm; mobile phase: [water (0.075% trifluoroacetic acid)-acetonitrile]: acetonitrile: 15%-45%, and purified to obtain the trifluoroacetate salt of compound 4A. MS m/z=647.3 [M+1]⁺. ¹H NMR (400 MHZ, CD₃OD) δ ppm 6.94 (d, J=8.5 Hz, 1H), 5.21 (br dd, J=3.9, 11.1 Hz, 1H), 4.79-4.49 (m, 4H), 4.47-4.32 (m, 1H), 4.24-4.02 (m, 3H), 3.86 (br d, J=13 9 Hz, 1H), 3.76-3.65 (m, 2H), 3.65-3.55 (m, 1H), 3.54-3.46 (m, 1H), 3.41-3.35 (m, 1H), 3.20-3.08 (m, 1H), 3.01-2.89 (m, 1H), 2.71 (br dd, J=6.5, 13.4 Hz, 1H), 2.41-2.19 (m, 8H), 2.19-1.90 (m, 8H).

### Step 7

Compound 4-4B (51 mg, 237 μmol) was dissolved in THF (3.0 mL), lithium tetrahydroaluminum (2.5 M, 0.15 mL) was added dropwise at 0° C., and the reaction solution was heated to 25° C. for 1 hour. Add 0.02 mL of water, 0.02 mL of 15% aqueous NaOH solution and 0.06 mL of water dropwise at 0° C., and stir for 0.5 hours. The mixture was filtered, the filter cake was washed with 10 mL of ethyl acetate, and the filtrate was concentrated under reduced pressure to obtain compound 4-5B, MS m/z=188.2 [M+1]⁺.

### Step 8

Compound 4-2 (100 mg, 116 μmol), compound 4-5B (41 mg, 219 μmol), 4 Å molecular sieve (70 mg) and sodium tert-butanol (80 mg, 832 μmol) were added to toluene (15 mL), and the temperature was increased to 100° C. for 6 hours. The reaction solution was filtered, the filter cake was washed with 10 mL of ethyl acetate, and the filtrate was concentrated under reduced pressure to obtain the crude product. The crude product was purified by preparing a thin layer of chromatographic separation (dichloromethane:methanol=20:1) to obtain compound 4-6B, MS m/z=987.4 [M+1]⁺.

### Step 9

Compounds 4-6B (70 mg, 70.9 μmol) were dissolved in dichloromethane (10.0 mL), to which trifluoroacetic acid (3.07 g, 26.9 mmol, 2 mL) was added, and the reaction solution was reacted at 25° C. for 12 hours. The reaction solution was concentrated under reduced pressure to obtain the crude product. The crude product was prepared for high performance liquid chromatography separation (column: Phenomenex luna C18 150*25 mm*10 μm; mobile phase: [water (0.075% trifluoroacetic acid)-acetonitrile]; acetonitrile: 15%-45%, and purified to obtain the trifluoroacetate salt of compound 4B. MS m/z=647.3 [M+1]⁺. ¹H NMR (400 MHZ, CD₃OD) δ ppm 6.94 (d, J=8.5 Hz, 1H), 5.21 (br dd, J=4.1, 10.6 Hz, 1H), 4.78-4.64 (m, 4H), 4.54 (br d, J=11.8 Hz, 1H), 4.42-4.32 (m, 1H), 4.21-4.11 (m, 2H), 3.88-3.54 (m, 6H), 3.40-3.34 (m, 2H), 2.96 (br dd, J=4.0, 17.9 Hz, 1H), 2.55-2.37 (m, 2H), 2.36-1.89 (m, 14H).

### Example 5

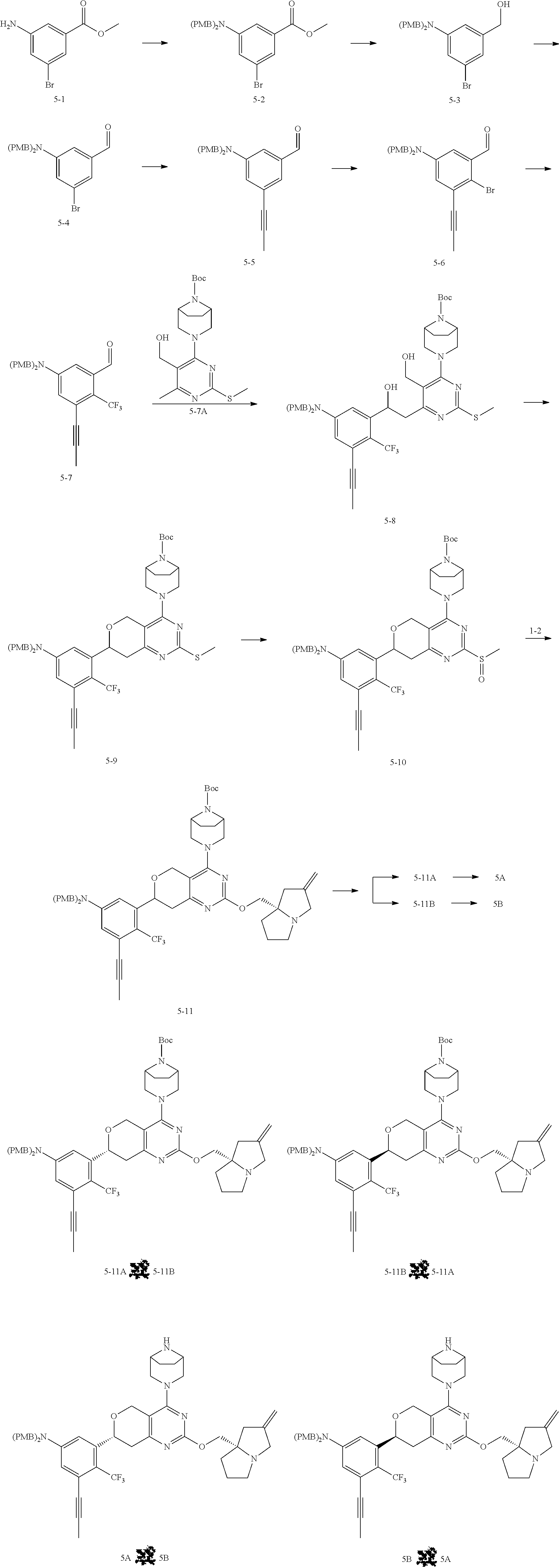

### Step 1

Compound 5-1 (10.0 g, 43.5 mmol) was dissolved in DMF (4 mL), then potassium carbonate (15.0 g, 109 mmol) and p-methoxybenzyl chloride (16.3 g, 104 mmol, 14.2 mL) were added and reacted at 80° C. for 12 hours. After the reaction solution was reduced to room temperature, 200 mL ethyl acetate was added to the reaction solution, washed with 200 mL water and 100 mL saturated salt water, anhydrous sodium sulfate was dried, filtered, and reduced pressure was obtained The crude product was purified by column chromatography (petroleum ether:ethyl acetate=30:1) to obtain compound 5-2. ¹H NMR (400 MHZ, CDCl₃) δ ppm 7.48 (t, J=1.31 Hz, 1H), 7.41 (dd, J=2.38, 1.13 Hz, 1H), 7.13 (d, J=8.50 Hz, 4H), 7.06-7.02 (m, 1H), 6.92-6.83 (m, 4H), 4.56 (s, 4H), 3.86 (s, 3H) 3.81 (s, 6H).

### Step 2

Compound 5-2 (17.5 g. 37.2 mmol) was dissolved in anhydrous THF (200 mL), then LiAlH₄ (2.5 M, 30 mL) was added at 0° C., and then reacted at 25° C. for 1 hour. The temperature of the reaction solution was reduced to room temperature, and 2.85 mL of H₂O, 2.85 mL of 15% NaOH aqueous solution and 8.6 mL of H₂O were added dropwise slowly under nitrogen flow. The solid produced was filtered and the filtrate was concentrated under reduced pressure to obtain compound 5-3. ¹H NMR (400 MHZ, CDCl₃) δ ppm 7.03 (d. J=8.63 Hz, 4H), 6.82-6.67 (m, 6H), 6.57 (s, 1H), 4.44 (s, 6H), 3.71 (s, 6H).

### Step 3

Compound 5-3 (15.0 g, 33.9 mmol) was dissolved in THF (150 mL), nitrogen replacement was performed for three times, MnO₂ (60.0 g, 690 mmol) was added, and the reaction solution was cooled at 75° C. for 12 hours, filtered, and the filtrate was concentrated under reduced pressure to obtain the crude product. The crude product was purified by column chromatography (petroleum ether: ethyl acetate=20:1) to obtain compound 5-4. ¹H NMR (400 MHZ, CDCl₃) δ ppm 9.79 (s, 1H), 7.16-7.09 (m, 6H), 6.92-6.86 (m, 5H), 4.59 (s, 4H), 3.81 (s, 6H).

### Step 4

Compounds 5-4 (7.20 g, 16.4 mmol), 1-propynyltri-N-butyltin (5.38 g, 16.4 mmol), dichlorobis [di-tert-butyl-(4-dimethylaminophenyl)phosphine]palladium (II) (57.9 mg, 81.8 μmol) were dissolved in anhydrous toluene (170 mL), nitrogen was replaced three times, and then reacted at 110° C. for 12 hours. The reaction solution was concentrated under reduced pressure to obtain the crude product. The crude product was purified by column chromatography (petroleum ether:ethyl acetate=10:1) to obtain compound 5-5. MS m/z=400.3 [M+H]⁺, 1H NMR (400 MHZ, CDCl₃) δ ppm 9.82 (s, 1H), 7.21 (s, 1H), 7.18-7.15 (m, 1H), 7.12 (d, J=8.63 Hz, 4H), 7.03-7.01 (m, 1H), 6.87 (d, J=8.63 Hz, 4H), 4.58 (s, 4H), 3.81 (s, 6H), 2.02 (s, 3H).

### Step 5

Compounds 5-5 (4.60 g, 11.5 mmol) were dissolved in anhydrous DMF (50 mL), NBS (2.25 g, 12.7 mmol) was added, and reacted at room temperature for 0.5 hours. Water (150 mL) was added to the organic phase, extracted with ethyl acetate (50 mL) for 3 times, the organic phase washed for 3 times with saturated brine (50 mL), dried over anhydrous sodium sulfate, filtered, and the filtrate was concentrated under reduced pressure to obtain the crude product. The crude product was purified by column chromatography (petroleum ether:ethyl acetate=10:1) to obtain compounds 5-6. MS m/z=478.2 [M+H]⁺. 1H NMR (400 MHZ, CDCl₃) δ ppm 10.31 (s, 1H), 7.24 (d, J=3.38 Hz, 1H), 7.10 (d, J=8.63 Hz, 4H), 7.05 (d, J=3.38 Hz, 1H), 6.89-6.83 (m, 4H), 4.55 (s, 4H), 3.80 (s, 6H), 2.09 (s, 3H).

### Step 6

Compounds 5-6 (5.30 g. 11.1 mmol), methylene iodide (4.22 g, 22.2 mmol), 2,2-difluoro-2-fluorosulfonylacetate (8.09 g, 42.1 mmol) were dissolved in DMF (50 mL), nitrogen was replaced three times, and the solution was reacted at 110° C. for 2.5 hours. The reaction solution was cooled, filtered with diatomaceous earth, water (150 mL) was added to the organic phase, and extracted 3 times with EA (150 mL). The organic phase was washed three times with saturated brine (150 mL), dried over anhydrous sodium sulfate, filtered, and the filtrate was concentrated under reduced pressure to obtain the crude product. The crude product was purified by column chromatography (petroleum ether:ethyl acetate=20:1) to obtain compounds 5-7. MS m/z=468.2 [M+H]⁺. 1H NMR (400 MHZ, CDCl₃) δ ppm 10.30 (q, J=2.71 Hz, 1H), 7.25 (d, J=2.75 Hz, 1H), 7.10 (d, J=8.63 Hz, 4H), 7.00 (d, J=2.88 Hz, 1H), 6.90-6.85 (m, 4H), 4.60 (s, 4H), 3.81 (s, 6H), 2.05 (d, J=4.13 Hz, 3H).

### Step 7

TMP (2.45 g, 17.4 mmol, 2.94 mL) was dissolved in THF (20 mL), nitrogen was replaced three times, the temperature was reduced to −40° C., n-BuLi (2.5 M, 6.71 mL) was added slowly dropwise, after dropping, reacted at −40° C. for 30 minutes, the reaction system was cooled to −60° C., 5-7A (2.20 g, 5.78 mmol) of THF (20 mL) solution was added slowly dropwise to the above reaction solution, and reacted at −40° C. for 15 minutes, compound 5-7 (3.94 g, 6.94 mmol) was added into the above reaction solution in batches, and the reacted at room temperature for 2 hours. The reaction solution was quenched with saturated aqueous ammonium chloride (100 mL), and extracted twice with ethyl acetate (100 mL). The organic phase was washed twice with saturated brine (100 mL), dried with anhydrous sodium sulfate, filtered, and the filtrate was concentrated under reduced pressure to obtain the crude product. The crude product was purified by column chromatography (petroleum ether: ethyl acetate=2:1) to obtain compound 5-8, MS m/z=848.4 [M+H]⁺. ¹H NMR (400 MHZ, CDCl₃) δ ppm 7.15-6.99 (m, 5H), 6.79 (d, J=8.63 Hz, 4H), 6.75 (br d, J=2.38 Hz, 1H), 5.39 (br d, J=8.63 Hz, 1H), 4.84-4.40 (m, 5H), 4.38-4.12 (m, 5H), 3.72 (s, 6H), 3.42-3.11 (m, 2H), 3.06-2.86 (m, 1H), 2.70-2.51 (m, 1H), 2.40 (s, 3H), 2.36-2.23 (m, 1H), 1.94 (s, 2H), 1.85-1.74 (m, 3H), 1.66 (br d, J=9.13 Hz, 1H), 1.42 (s, 9H).

### Step 8

Compounds 5-8 (324 mg. 390 μmol) were dissolved in anhydrous toluene (23 mL), cyanomethylene tributyl phosphate (1.30 g, 5.40 mmol) was added, and nitrogen was replaced for three times and reacted at 110° C. for 12 hours. The reaction solution was concentrated under reduced pressure to obtain the crude product. Crude product was purified by column chromatography (petroleum ether: Ethyl acetate=10:1) and preparation of high performance liquid chromatography separation (chromatographic column: Phenomenex luna C18 150*25 mm*10 μm; mobile phase: [water (0.05% hydrochloric acid)-acetonitrile]; acetonitrile: 55%-85%); concentrated under reduced pressure to obtain the hydrochloride salt of compound 5-9. MS m/z=830.5 [M+H]⁺.

### Step 9

Compound 5-9 (324 mg, hydrochloride) was dissolved in anhydrous dichloromethane (3 mL), metachloroperoxybenzoic acid (83.2 mg, 410 μmol, 85% purity) was added, and then reacted at room temperature for 0.5 hours. The reaction solution was concentrated under reduced pressure and sodium bicarbonate (10 mL), sodium sulfite (10 mL) was quenched, extracted twice with DCM (50 mL), washed with 50 mL of saturated salt, dried with anhydrous sodium sulfate, filtered, and the filtrate was concentrated under reduced pressure to obtain compound 5-10. MS m/z=846.5 [M+H]⁺.

### Step 10

Compound 5-10 (280 mg, 331 μmol) was dissolved in anhydrous toluene (20 mL), 4 Å molecular sieve (150 mg, 2.34 mmol), compound 1-2 (203 mg, 1.32 mmol), sodium tert-butoxide (127 mg, 1.32 mmol) was added, and then reacted at 100° C. for 12 hours. The reaction solution was filtered and concentrated under reduced pressure to obtain the crude product. Crude product was purified by column chromatography (dichloromethane:methanol=20:1) and prepared for high performance liquid chromatography separation (chromatographic column. Phenomenex luna C18 150*30 mm*10 μm; mobile phase: [water (0.225% formic acid)-acetonitrile]; (acetonitrile): 48%-78%) and concentrated under reduced pressure to obtain the formate salt of compound 5-11. MS m/z=935.7 [M+H]⁺. The SFC separation was then prepared (chiral column: DAICEL CHIRALCEL OD (250 mm*30 mm, 10 μm); mobile phase: [supercritical carbon dioxide-acetonitrile/isopropanol (0.1% ammonia water)]; acetonitrile/isopropanol (0.1% ammonia water): 45%-45%, and compound 5-11A and compound 5-11B were obtained after concentration under reduced pressure. After analysis, SFC (chiral column: DAICEL CHIRALCEL OD-3 (50 mm*4.6 mm, 3 μm; mobile phase: [supercritical carbon dioxide-methanol (0.05% diethylamine)]; methanol (0.05% diethylamine) %: 40%, compound 5-11A, Rt=0.657 min, ee value 99%, MS m/z=935.6 [M+H]⁺; compound 5-11B, Rt=1.848 min, ee value 99% MS m/z=935.5 [M+H]⁺.

### Step 11

Compound 5-11A (105 mg, 112 μmol) was dissolved in TFA (1 mL) and then reacted at 50° C. for 2 hours. The reaction solution was concentrated under reduced pressure to obtain the crude product, and the crude product was prepared for high performance liquid chromatography separation (column: Phenomenex luna C18 150*25 mm*10 μm; mobile phase: [water (0.075% trifluoroacetic acid-acetonitrile]; acetonitrile: 12%-42%). After lyophilization, the trifluoroacetate of compound 5A was obtained. MS m/z=595.4 [M+H]⁺. H NMR (400 MHZ, CD₃OD) δ ppm 6.96 (d, J=2.4 Hz, 1H), 6.75 (d, J=2.4 Hz, 1H), 5.34-5.28 (m, 2H), 5.11 (dd, J=2.8, 10.8 Hz, 1H), 4.99-4.92 (m, 1H), 4.82-4.75 (m, 1H), 4.57 (d, J=1.6 Hz, 2H), 4.44-4.30 (m, 2H), 4.21-4.11 (m, 2H), 3.96-3.83 (m, 2H), 3.81-3.66 (m, 2H), 3.33 (br s, 1H), 3.24 (td,/=7.2, 11.6 Hz, 1H), 3.05 (br s, 1H), 3.03-2.98 (m, 1H), 2.87-2.74 (m, 2H), 2.41-2.32 (m, 1H), 2.30-2.19 (m, 2H), 2.18-2.05 (m, 4H), 2.05-1.93 (m, 4H).

Compounds 5-11B (110 mg, 118 μmol) were dissolved in TFA (1 mL) and then reacted at 50° C. for 2 hours. The reaction solution was concentrated under reduced pressure to obtain the crude product. The crude product was prepared for high performance liquid chromatography separation (column: Phenomenex luna C18 150*25 mm*10 μm; mobile phase: [water (0.075% trifluoroacetic acid-acetonitrile]; acetonitrile: 12%-42%, and after lyophilization, the trifluoroacetate salt of compound 5B was obtained. MS m/z=595.4 [M+H]⁺. ¹H NMR (400 MHZ, CD₃OD) δ ppm 6.97 (d, J=2.4 Hz, 1H), 6.76 (d, J=2.4 Hz, 1H), 5.30 (br d, J=8.0 Hz, 2H), 5.11 (br dd, J=10.8, 2.8 Hz, 1H), 4.98-4.94 (m, 1H), 4.84-4.77 (m, 1H), 4.66-4.55 (m, 2H), 4.46 (br d, J=14.0 Hz, 1H), 4.34 (br d, J=14.0 Hz, 1H), 4.17 (br d, J=16.0 Hz, 2H), 3.96-3.85 (m, 2H), 3.82-3.68 (m, 2H), 3.37 (br d, J=14.0 Hz, 1H), 3.23 (td, J=7.2, 11.6 Hz, 1H), 3.03 (br d, J=16.4 Hz, 2H), 2.89-2.75 (m, 2H), 2.42-2.33 (m, 1H), 2.28-2.20 (m, 2H), 2.20-2.09 (m, 4H), 2.06-1.93 (m, 4H).

### Example 7

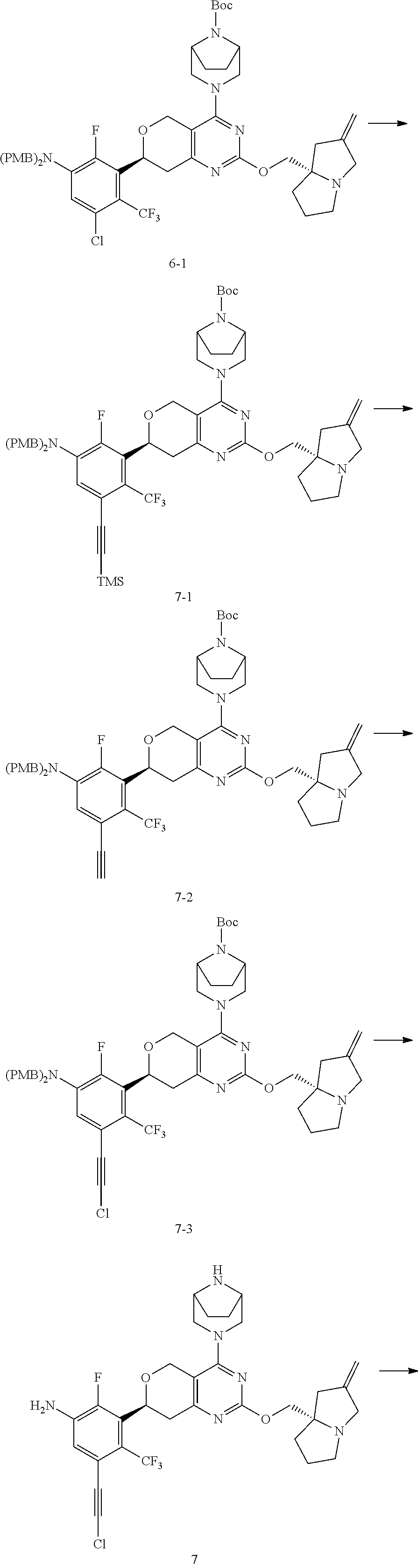

### Step 1

Compound 7-1 (1.20 g. 1.26 mmol) was dissolved in anhydrous toluene (12.0 mL), followed by the addition of tributyl (trimethylsilylethynyl) tin (2.94 g, 7.58 mmol) and dichlorobis [di-tert-butyl-(4-dimethylaminophenyl)phosphine]palladium (II) (537 mg, 758 μmol). The reaction was performed at 110° C. for 20 hours. The reaction solution was concentrated under reduced pressure to obtain the crude product. The crude product was purified by column chromatography (petroleum ether: ethyl acetate=2:1) to obtain compounds 7-1. MS m/z=1011.6 [M+1]⁺.

### Step 2

Compound 7-2 (1.00 g, 989 μmol) was dissolved in anhydrous tetrahydrofuran (10.0 mL), and then tetrabutylammonium fluoride (1 M, 989 μL) was added. React at 25° C. for 4 hours. 30.0 mL of water was added to the reaction solution, extracted with ethyl acetate (150 mL), dried with the organic phase, and concentrated under reduced pressure to obtain the crude product. The crude product was purified by column chromatography (petroleum ether:ethyl acetate=1:1) to obtain compounds 7-2. MS m/z=939.6 [M+1]⁺.

### Step 3

Compound 7-2 (310 mg, 330.12 μmol) was dissolved in anhydrous acetone (5 mL), followed by addition of AgNO₃ (350 mg, 2.06 mmol), NCS (220.41 mg, 1.65 mmol). At 25° C. Reaction for 10 hours. The reaction solution was concentrated under reduced pressure to obtain the crude product. The crude product was purified by preparing a thin layer of chromatographic separation (dichloromethane:methanol=10:1) to obtain compound 7-3. MS m/z=973.4 [M+1]⁺.

### Step 4

Compound 7-3 (12.0 mg, 12.3 μmol) was dissolved in dichloromethane (1 mL), trifluoroacetic acid (153 mg, 1.35 mmol, 0.1 mL) was added, and the solution was reacted at 25° C. for 1 hour. The reaction solution was concentrated under reduced pressure to obtain the crude product. The crude product was prepared for high performance liquid chromatography separation (column: Phenomenex luna C18 150*25 mm*10 μm; mobile phase: [water (0.075% trifluoroacetic acid)-acetonitrile]; acetonitrile: 18%-48%. After lyophilization, the trifluoroacetate of compound 7 was obtained. MS m/z=633.2 [M+1]⁺. ¹H NMR (400 MHZ, CD₃OD) δ ppm 7.00 (d, J=8.5 Hz, 1H), 5.33-5.30 (m, 2H), 5.21-5.18 (m, 1H), 4.85 (s, 4H), 4.76-4.71 (m, 1H), 4.54 (s, 2H), 4.35-4.30 (m, 2H), 4.17-4.12 (m, 2H), 3.93 (d, J=14.0 Hz, 1H), 3.86-3.70 (m, 2H), 3.68-3.64 (m, 1H), 3.07-2.88 (m, 2H), 2.82-2.73 (m, 1H), 2.37-2.29 (m, 1H), 2.28-2.18 (m, 2H), 2.17-2.05 (m, 4H), 2.01-1.92 (m, 1H).

### Example 8

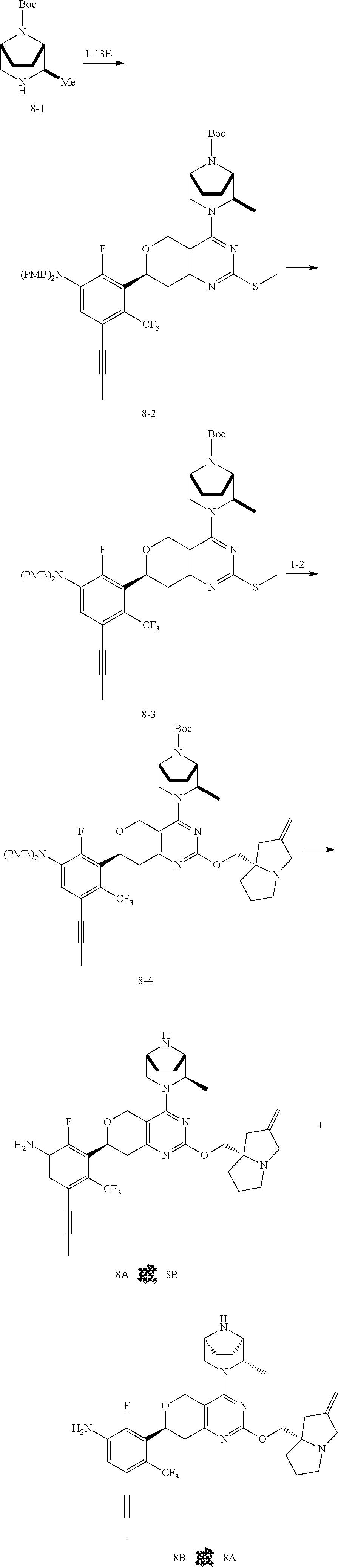

### Step 1

Compound 1-13B (100 mg, 127 μmol), compound 8-1 (63.4 mg, 280 μmol), triethylamine (509 μmol, 70.9 μL) were dissolved in DMF (1.00 mL), nitrogen was replaced three times, and the reaction was performed at 50° C. for six hours. 10.0 mL of water was added to the reaction solution, and extracted three times with ethyl acetate (20.0 mL). The organic phase was washed with saturated brine (50.0 mL), dried over anhydrous sodium sulfate, filtered, and concentrated under reduced pressure to obtain the crude product. The crude product was purified to compound 8-2 by preparation of a thin layer of chromatography (petroleum ether:ethyl acetate=3:1). MS m/z=862.4 [M+H]⁺.

### Step 2

Compound 8-2 (60.0 mg, 69.6 μmol) was dissolved in anhydrous methylene chloride (1.00 mL), metachloroperoxybenzoic acid (15.5 mg, 76.6 μmol 85.0% purity) was added in batches at 0° C., and then reacted at room temperature for 1 hour. 10.0 mL of sodium bicarbonate solution and 10.0 mL of sodium sulfate solution were added to the reaction solution, and extracted with dichloromethane (30.0 mL) for three times. The organic phase was washed with saturated brine (50.0 mL), dried over anhydrous sodium sulfate, filtered, and concentrated under reduced pressure to obtain the crude product. The crude product was purified by preparation of thin layer of chromatography (petroleum ether:ethyl acetate=2:1) to obtain compound 8-3. MS m/z=878.3 [M+H]⁺.

### Step 3

Compound 8-3 (35.0 mg, 39.9 μmol) was dissolved in anhydrous toluene (1.00 mL), compound 1-2 (12.2 mg, 79.7 μmol), tert-butanol sodium (15.3 mg, 159 μmol) and 4 Å molecular sieve (18.0 mg), and the reaction was carried out at 100° C. for 6 hours after nitrogen replacement. The reaction solution was concentrated under reduced pressure to obtain the crude product. The crude product was purified by preparation of a thin layer of chromatography (pure ethyl acetate) to obtain compound 8-4. MS m/z=967.4 [M+H]⁺.

### Step 4

Compounds 8-4 (30.0 mg, 31.0 μmol) were dissolved in anhydrous methylene chloride (0.5 mL), trifluoroacetic acid (1.35 mmol, 100 μL) was added, and then reacted at room temperature for 2 hours. The reaction solution was concentrated under reduced pressure to obtain the crude product, and the crude product was prepared for high performance liquid chromatography separation (column: Phenomenex luna C18 150*25 mm*10 μm; mobile phase: [water (0.075% trifluoroacetic acid-acetonitrile]; acetonitrile: 16%-46%. After lyophilization, the trifluoroacetate of compound 8A and the trifluoroacetate of 8B were obtained.

Trifluoroacetate of Compound 8A: MS m/z=627.3 [M+H]⁺, ¹H NMR (400 MHZ, CD₃OD) δ ppm 6.92 (d, J=8.5 Hz, 1H), 5.31 (br d, J=8.5 Hz, 2H), 5.19 (br dd, J=3.6, 11.8 Hz, 1H), 4.97-4.93 (m, 1H), 4.73 (br d, J=14.6 Hz, 1H), 4.65-4.45 (m, 2H), 4.34 (br d, J=14.4 Hz, 1H), 4.14 (br d, J=5.5 Hz, 1H), 4.03-3.89 (m, 3H), 3.81-3.70 (m, 1H), 3.42-3.34 (m, 1H), 3.29-3.23 (m, 1H), 3.22-3.15 (m, 1H), 3.07-2.91 (m, 3H), 2.81 (br d, J=16.0 Hz, 1H), 2.36 (br dd, J=5.9, 11.4 Hz, 2H), 2.31-2.10 (m, 5H), 2.06-1.93 (m, 4H), 1.15 (d, J=6.0 Hz, 3H).

Trifluoroacetate of Compound 8B, MS m/z=627.3 [M+H]⁺, ¹H NMR (400 MHZ, CD₃OD) δ ppm 6.93 (d, J=8.5 Hz, 1H), 5.31 (br d, J=8.0 Hz, 2H), 5.10-4.99 (m, 2H), 4.75 (s, 1H), 4.57 (s, 2H), 4.33 (br d, J=14.3 Hz, 1H), 4.21-4.09 (m, 1H), 3.98-3.84 (m, 3H), 3.82-3.71 (m, 1H), 3.45 (br dd, J=11.2, 17.7 Hz, 1H), 3.28-3.18 (m, 2H), 3.15-3.01 (m, 2H), 2.92-2.77 (m, 2H), 2.40-2.11 (m, 7H), 2.03-1.92 (m, 4H), 1.02 (d, J=6.3 Hz, 3H).

### Example 9

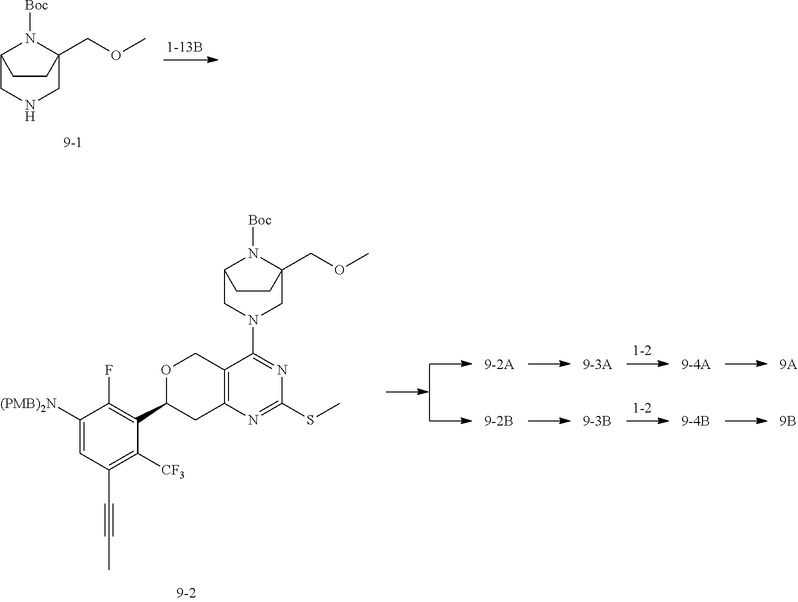

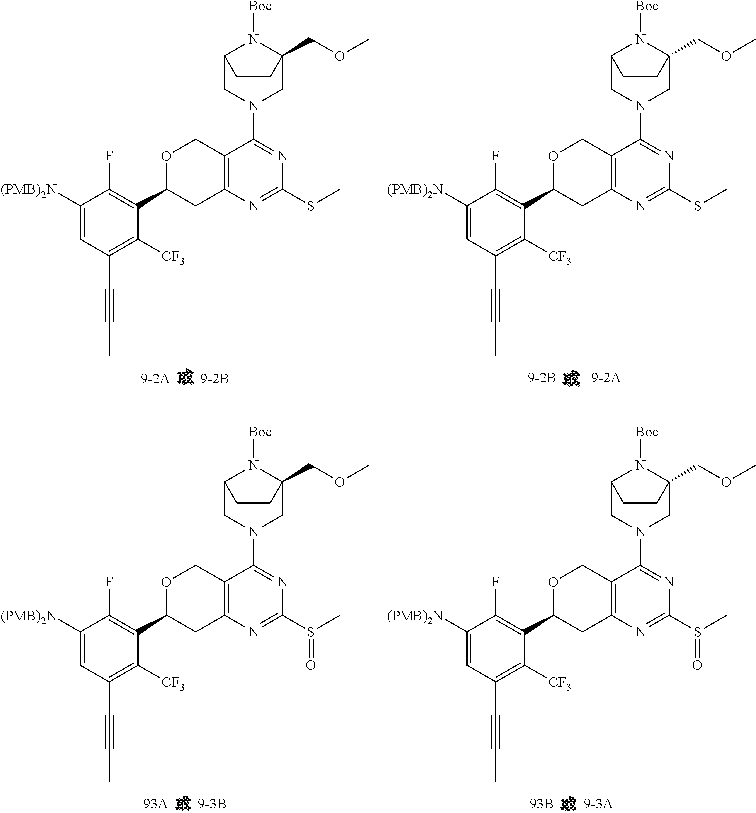

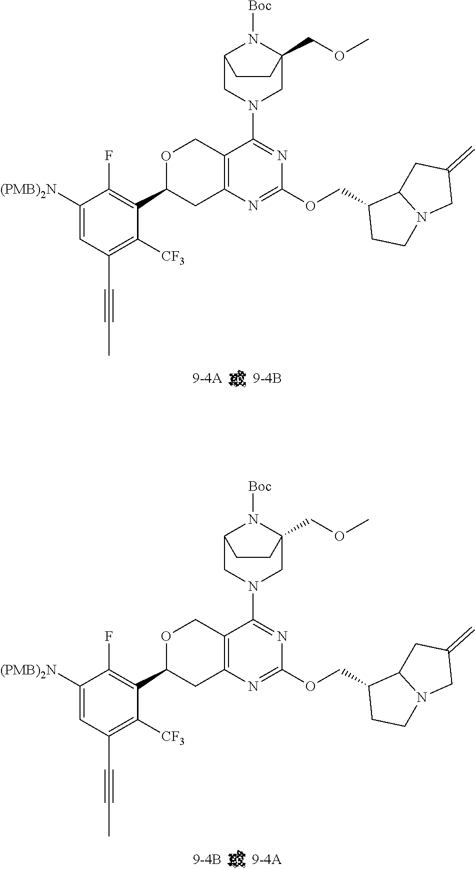

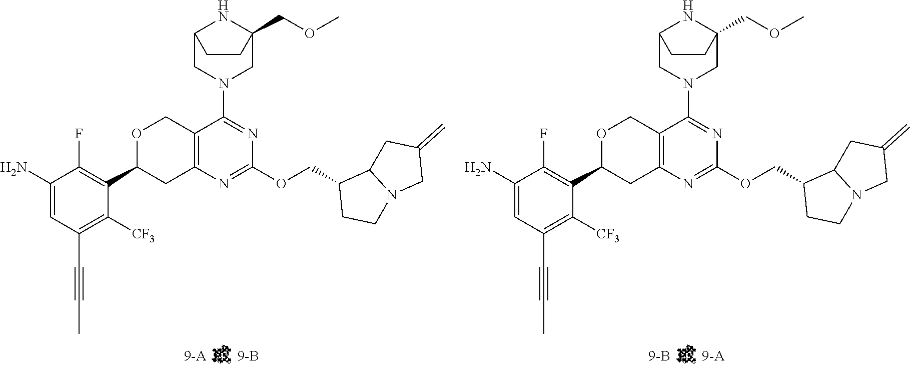 [SMILES: C=C1CC2[C@@H](COc3nc4c(c(N5CC6CC[C@@](COC)(C5)N6)n3)CO[C@H](c3c(F)c(N)cc(C#CC)c3C(F)(F)F)C4)CCN2C1 ; C=C1CC2[C@@H](COc3nc4c(c(N5CC6CC[C@](COC)(C5)N6)n3)CO[C@H](c3c(F)c(N)cc(C#CC)c3C(F)(F)F)C4)CCN2C1]

### Step 1

Compound 1-13B (49.0 mg, 62.4 μmol) and compound 9-1 (48.0 mg, 187 μmol) were dissolved in dichloromethane (1.00 mL), to which it was added with N,N-diisopropylethylamine (54.3 μL), and the reaction solution was reacted at 25° C. for 12 hours. The reaction solution was concentrated under reduced pressure to obtain crude product. The crude product was purified by column chromatography (petroleum ether:ethyl acetate=10:1) to obtain compound 9-2. MS m/z=892.4 [M+1]⁺. The SFC was further prepared for separation (chiral column: (column: (s,s) WHELK-01 (250 mm*30 mm, 10 μm); mobile phase: [supercritical carbon dioxide-acetonitrile/isopropanol (0.1% ammonia water)]; acetonitrile/isopropanol (0.1% ammonia water): 50%-50%. Compound 9-2A and compound 9-2B were obtained after concentration under reduced pressure. Analysis SFC: (chiral column: (s,s) WHELK-01 (50 mm*4.6 mm, 3.5 μm); mobile phase: [supercritical carbon dioxide-isopropanol (0.05% diethylamine)]; isopropanol (0.05% diethylamine) %: 40%, compound 9-2A, Rt=1.768 min, ee value 99%; MS m/z=892.4 [M+H]⁺; compound 9-2B, Rt=2.286 min, 99% ee, MS m/z=892.4 [M+H]⁺.

### Step 2

Compound 9-2A (30.0 mg, 33.6 μmol) was dissolved in dichloromethane (1.00 mL), to which metachloroperoxybenzoic acid (8.19 mg, 40.4 μmol, 85.0% purity) was added, and the solution was reacted at 25° C. for 2 hours. The reaction solution was quenched with a saturated sodium sulfite solution, the organic phase was collected and concentrated under reduced pressure to obtain a crude product. The crude product was purified by column chromatography (petroleum ether:ethyl acetate=1:1) to obtain compound 9-3A. m/z=908.4 [M+1]⁺.

With reference to step 2, compound 9-2A was replaced with compound 9-2B as the raw material to obtain compound 9-3B. m/z=908.4 [M+1]⁺.

### Step 3

Compound 9-3A (12.0 mg, 13.2 μmol), compound 1-2 (6.07 mg, 39.7 μmol), sodium tert-butanol (3.81 mg. 39.7 μmol) and 4 Å molecular sieve (12.0 mg) were added to toluene (1.00 mL), and the reaction solution was reacted at 110° C. for 12 hours. The reaction solution was cooled to room temperature, filtered, and the filtrate was concentrated to obtain the crude product. The crude product was purified by column chromatography (dichloromethane:methanol=10:1) to obtain compound 9-4A. MS m/z=997.6 [M+1]⁺.

With reference to step 3, compound 9-3A was replaced with compound 9-3B as the raw material to obtain compound 9-4B. MS m/z=997.6 [M+1]⁺.

### Step 4

Compound 9-4A (10.0 mg, 10.0 μmol) was dissolved in dichloromethane (1.00 mL), to which trifluoroacetic acid (0.30 mL) was added, and the reaction solution was reacted at 25° C. for 1 hour. The reaction solution was concentrated under reduced pressure to obtain crude product. The crude product was prepared for high performance liquid chromatography separation (column: Phenomenex luna C18 150*25 mm*10 μm; mobile phase: [water (0.075% trifluoroacetic acid)-acetonitrile]; acetonitrile: 15%-45% purification to obtain 9A of trifluoroacetate. MS m/z=657.4 [M+1]⁺, ¹H NMR (400 MHZ, CD₃OD) δ ppm 6.97-6.92 (m, 1H), 5.33 (br d, J=78 Hz, 2H), 5.28-5.17 (m, 1H), 4.76-4.72 (m, 1H), 4.56 (s, 2H), 4.41-4.28 (m, 2H), 4.22-4.15 (m, 1H), 3.99-3.91 (m, 1H), 3.79-3.63 (m, 5H), 3.52-3.48 (m, 3H), 3.35 (br d, J=1.8 Hz, 1H), 3.31-3.23 (m, 3H), 3.09-2.79 (m, 3H), 2.40-2.10 (m, 6H), 2.04 (s, 3H), 2.01-1.88 (m, 2H).

Compounds 9-4B (13 0 mg, 13.0 μmol) were dissolved in dichloromethane (1.00 mL), to which trifluoroacetic acid (0.30 mL) was added, and the reaction solution was reacted at 25° C. for 1 hour. The reaction solution was concentrated under reduced pressure to obtain crude product. Preparation of crude product High performance liquid chromatography separation (column: Phenomenex luna C18 150*25 mm*10 μm; mobile phase: [water (0.075% trifluoroacetic acid)-acetonitrile]; acetonitrile: 15%-45% purification to obtain 9B of trifluoroacetate. MS m/z=657.5 [M+1]⁺, ¹H NMR (400 MHZ, CD₃OD) δ ppm 6.94 (d, J=8.5 Hz, 1H), 5.33 (br d, J=7.6 Hz, 2H), 5.26-5.18 (m, 1H), 4.78-4.73 (m, 1H), 4.57 (s, 2H), 4.39-4.28 (m, 2H), 4.17-4.12 (m, 1H), 3.94 (br d, J=14.4 Hz, 1H), 3.85-3.63 (m, 5H), 3.50 (s, 3H), 3.41-3.34 (m, 2H), 3.31-3.21 (m, 2H), 3.09-2.78 (m, 3H), 2.41-2.12 (m, 6H), 2.04 (s, 3H), 2.02-1.91 (m, 2H).

### Example 10

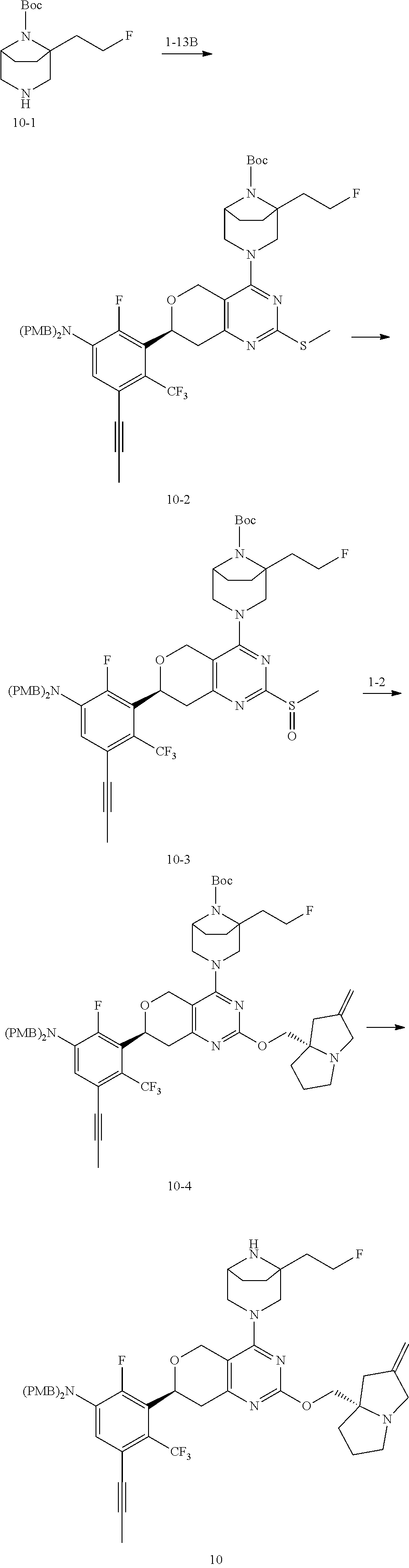

### Step 1

Compound 1-13B (49.0 mg, 62.4 μmol) and compound 10-1 (48.0 mg, 187 μmol) were dissolved in dichloromethane (1.00 mL), to which N,N-diisopropylethylamine (40.3 mg, 312 μmol, 54.3 μL) was added, and the solution was reacted under 25° C. for 12 hours. The reaction solution was concentrated directly to obtain crude product. The crude product was purified by column chromatography (petroleum ether:ethyl acetate=5:1) to obtain compound 10-2. MS m/z=894.4 [M+1]⁺.

### Step 2

Compound 10-2 (50.0 mg, 55.9 μmol) was dissolved in dichloromethane (1.00 mL), to which m-chloroperoxybenzoic acid (13.6 mg, 67.1 μmol, 85.0% purity) was added, and the reaction solution was reacted at 25° C. for 1 hour. The reaction solution was quenched with a saturated sodium sulfite solution, the aqueous phase was extracted with dichloromethane (10.0 mL*2), the organic phase was collected and concentrated to obtain the crude product. The crude product was purified by column chromatography (petroleum ether:ethyl acetate=1:1) to obtain compound 10-3. m/z=910.3 [M+1]⁺.

### Step 3

Compound 10-3 (30.0 mg, 32.9 μmol), compound 1-2 (15.2 mg, 98.9 μmol), sodium tert-butanol (9.50 mg, 98.9 μmol) and 4 Å molecular sieve (30.0 mg) were added to toluene (1.00 mL), and the reaction solution was reacted at 110° C. for 12 hours. The reaction solution was cooled to room temperature, filtered, and the filtrate was concentrated to obtain the crude product. The crude product was purified by column chromatography (dichloromethane:methanol=10:1) to obtain compound 10-4. MS m/z=999.6 [M+1]⁺.

### Step 4

Compound 10-4 (25.0 mg, 25.0 μmol) was dissolved in dichloromethane (1.00 mL), to which trifluoroacetic acid (0.30 mL) was added, and the reaction solution was reacted at 25° C. for 1 hour. The reaction solution was concentrated directly to obtain crude product. Preparation of crude product High performance liquid chromatography separation (column: Phenomenex luna C18 150*25 mm*10 μm; mobile phase: [water (0.075% trifluoroacetic acid)-acetonitrile]; acetonitrile: 12%-42% purification to obtain 10 trifluoroacetate salt. MS m/z=659.3 [M+1]⁺, ¹H NMR (400 MHZ, CD₃OD) δ ppm 6.92 (d, J=8.5 Hz, 1H), 5.54-5.52 (m, 1H), 5.31 (br d, J=7.6 Hz, 2H), 5.23-5.13 (m, 1H), 4.94-4.90 (m, 1H), 4.85-4.63 (m, 5H), 4.62-4.25 (m, 4H), 4.23-4.11 (m, 1H), 3.97-3.67 (m, 4H), 3.40-3.33 (m, 1H), 3.28-3.22 (m, 1H), 3.10-2.89 (m, 2H), 2.80 (br d, J=15.9 Hz, 1H), 2.44-2.33 (m, 2H), 2.27-2.11 (m, 5H), 2.02 (s, 3H), 1.98-1.91 (m, 1H).

### Example 11

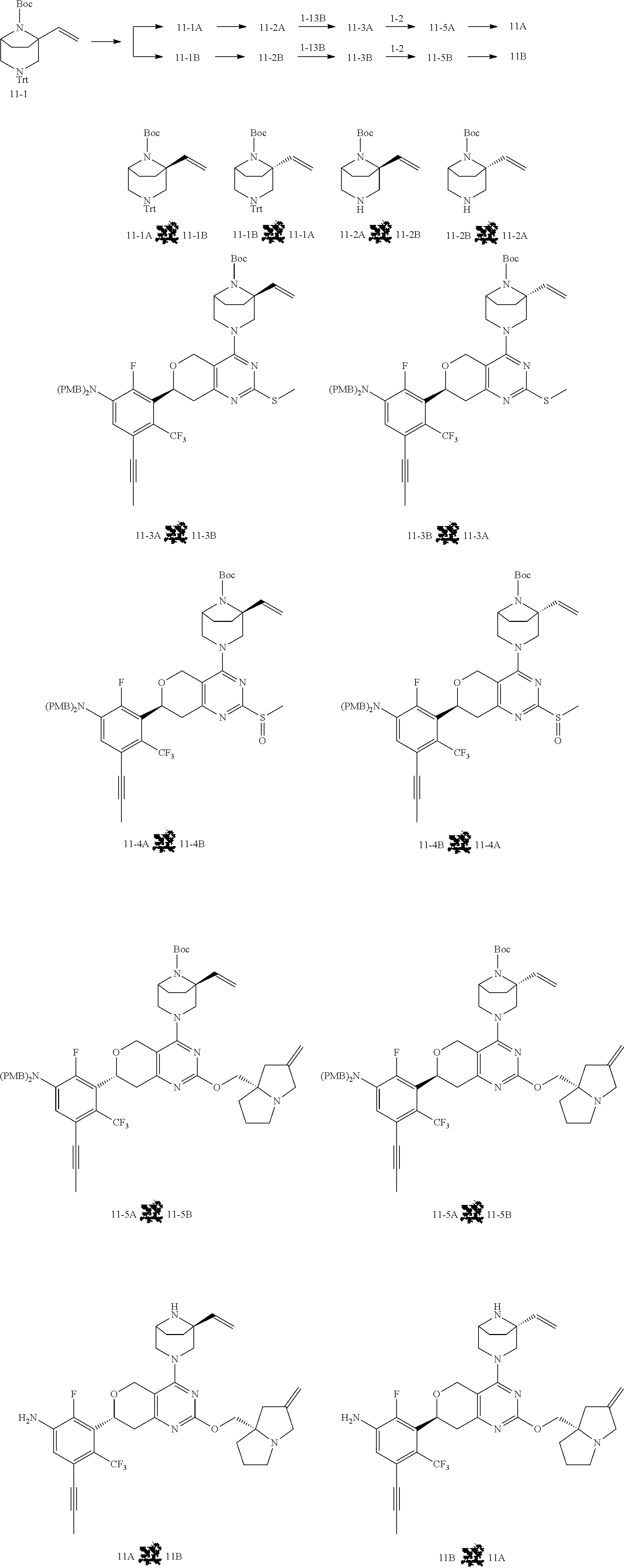

### Step 1

Compound 11-1 was prepared for SFC separation (chiral column: DAICEL CHIRALPAK AD (250 mm*30 mm, 10 μm; mobile phase: [supercritical carbon dioxide-acetonitrile/isopropanol (0.1% ammonia water)]; acetonitrile/isopropanol (0.1% ammonia water): 30%-30%. Compound 11-1A and compound 11-1B were obtained after concentration under reduced pressure. Analysis SFC: (chiral column: DAICEL CHIRALPAK AD (50 mm*4.6 mm, 3 μm); mobile phase: [supercritical carbon dioxide-isopropanol (0.05% diethylamine]; isopropanol (0.05% diethylamine) %: 5%-40%, compound 11-1A, Rt=1.308 min, ee value 99%; MS m/z=503.2 [M+Na]⁺; compound 11-1B, Rt=1.499 min, 99% ee, MS m/z=503.2 [M+Na]⁺.

### Step 2

Compound 11-1A (238 mg, 495 μmol) was dissolved in acetic acid (4.00 mL), and the acetic acid was concentrated under reduced pressure at 25° C. for 12 hours to remove the acetate salt of compound 11-2A.

With reference to Step 2, Compound 11-1B was used as the raw material to replace Compound 11-1A to obtain the acetate salt of Compound 11-2B.

### Step 3

Compounds 1-13B (200 mg, 254 μmol), compound 11-2A (182 mg, acetate) were dissolved in dichloromethane (3.00 mL), to which was added N,N-diisopropylethylamine (1.27 mmol, 222 μL), and the reaction solution was reacted at 25° C. for 12 hours. The reaction solution was concentrated directly to obtain crude product. The crude product was purified by column chromatography (petroleum ether:ethyl acetate=5:1) to obtain compound 11-3A. MS m/z=874.4 [M+1]⁺.

With reference to step 3, the acetate salt of compound 11-2B was used as the raw material to replace the acetate salt of compound 11-2A to obtain compound 11-3B. MS m/z=874.4 [M+1]⁺.

### Step 4

Compound 11-3A (180 mg, 206 μmol) was dissolved in dichloromethane (3.00 mL), to which metachloroperoxybenzoic acid (43.9 mg, 216 μmol, 85.0% purity) was added, and the reaction solution was reacted at 25° C. for 2 hours. Add 10.0 mL of sodium bicarbonate solution and 10.0 mL of sodium sulfite solution to the reaction solution, extract the aqueous phase with dichloromethane (30.0 mL*2), collect the organic phase and concentrate to obtain the crude product. The crude product was purified by column chromatography (petroleum ether:ethyl acetate=1:1) to obtain compound 11-4A.

With reference to step 4, compound 11-3A was replaced with compound 11-3B as the raw material to obtain compound 11-4B.

### Step 5

Compound 11-4A (140 mg, 157 μmol), compound 1-2 (48.2 mg, 315 μmol), sodium tert-butoxide (60.5 mg, 629 μmol) and 4 Å molecular sieve (70.0 mg) were added to toluene (3.00 mL), and the solution was reacted at 110° C. for 12 hours. The reaction solution was cooled to room temperature, filtered, and the filtrate was concentrated to obtain the crude product. The crude product was purified by column chromatography (petroleum ether:ethyl acetate=1:2) to obtain compound 11-5A. MS m/z=979.5 [M+1]⁺.

With reference to step 4, compound 11-4A was replaced with compound 11-4B as the raw material to obtain compound 11-5B. MS m/z=979.5 [M+1]⁺.

### Step 6

Compound 11-5A (72.0 mg, 73.5 μmol) was dissolved in dichloromethane (1.00 mL), to which trifluoroacetic acid (0.20 mL) was added, and the reaction solution was reacted at 25° C. for 2 hours. The reaction solution was concentrated directly to obtain crude product. Preparation of crude product for high performance liquid chromatography separation (column: Phenomenex luna C18 150*25 mm*10 μm; mobile phase: [water (0.075% trifluoroacetic acid)-acetonitrile]; acetonitrile: 16%-46% purification to obtain 11A of trifluoroacetate. MS m/z=639.3 [M+H]⁺, ¹H NMR (400 MHz, CD₃OD) δ ppm 6.92 (d, J=8.6 Hz, 1H), 6.15 (dd, J=11.2, 17.7 Hz, 1H), 5.61-5.46 (m, 2H), 5.31 (br d, J=7.5 Hz, 2H), 5.25-5.17 (m, 1H), 5.09-4.97 (m, 1H), 4.77-4.72 (m, 2H), 4.55 (s, 2H), 4.39-4.22 (m, 3H), 3.98-3.70 (m, 3H), 3.55 (d, J=13.6 Hz, 1H), 3.28-3.21 (m, 2H), 3.09-2.90 (m, 2H), 2.79 (br d, J=16.3 Hz, 1H), 2.40-2.05 (m, 8H), 2.02 (s, 3H).

Compounds 11-5B (64.0 mg, 65.4 μmol) were dissolved in dichloromethane (1.00 mL), to which trifluoroacetic acid (0.20 mL) was added, and the reaction solution was reacted at 25° C. for 2 hours. The reaction solution was concentrated directly to obtain crude product. Preparation of crude product for high performance liquid chromatography separation (column: Phenomenex luna C18 150*25 mm*10 μm; mobile phase: [water (0.075% trifluoroacetic acid)-acetonitrile]; acetonitrile: 16%-46% purification to obtain 11B of trifluoroacetate. MS m/z=639.3 [M+H]⁺, ¹H NMR (400 MHz, CD₃OD) δ ppm 6.92 (d, J=8.5 Hz, 1H), 6.18 (dd, J=11.2, 17.7 Hz, 1H), 5.66-5.48 (m, 2H), 5.31 (br d, J=7.4 Hz, 2H), 5.20 (br dd, J=4.1, 10.9 Hz, 1H), 4.95-4.91 (m, 1H), 4.78-4.71 (m, 2H), 4.55 (s, 2H), 4.45-4.16 (m, 3H), 3.98-3.62 (m, 4H), 3.27-3.22 (m, 2H), 3.09-2.88 (m, 2H), 2.79 (br d, J=16.3 Hz, 1H), 2.56-2.03 (m, 8H), 2.02 (s, 3H).

### Example 12

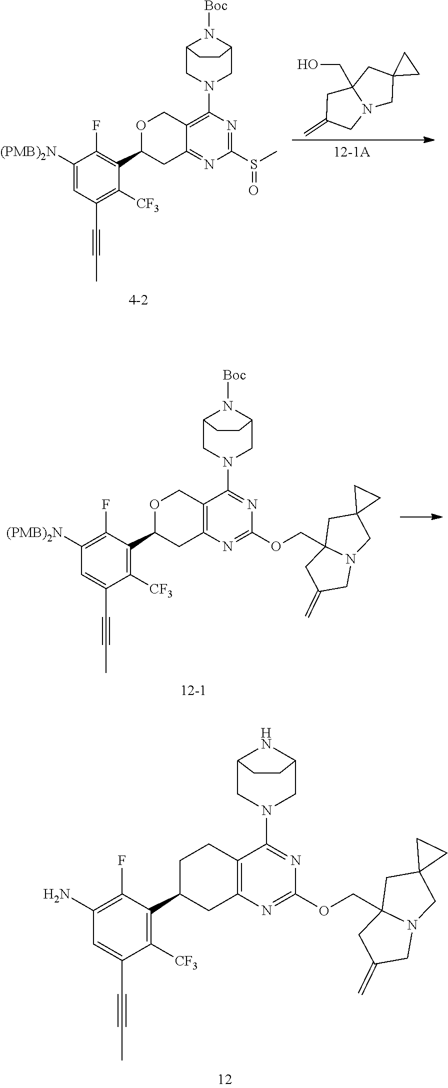

### Step 1

Compound 4-2 (100 mg, 115 μmol), compound 12-1A (83.0 mg, 463 μmol), sodium tert-butoxide (44.5 mg, 463 μmol) and 4 Å molecular sieve (100.0 mg) were added to toluene (5.00 mL), and the reaction solution was reacted at 110° C. for 12 hours. The reaction solution was cooled to room temperature, filtered, and the filtrate was concentrated to obtain the crude product. The crude product was purified by column chromatography (dichloromethane:methanol=10:1) to obtain compound 12-1. MS m/z=979.5 [M+1]⁺.

### Step 2

Compound 12-1 (90.0 mg, 91.9 μmol) was dissolved in dichloromethane (1.00 mL), to which trifluoroacetic acid (0.30 mL) was added, and the reaction solution was reacted at 25° C. for 2 hours. The reaction solution was concentrated directly to obtain crude product. Preparation of crude product for high performance liquid chromatography separation (column: Phenomenex luna C18 150*25 mm*10 μm; mobile phase: [water (0.075% trifluoroacetic acid)-acetonitrile]; acetonitrile: 16%-46% purification to obtain 12 of trifluoroacetate. MS m/z=639.4 [M+H]⁺, ¹H NMR (400 MHZ, D₂O) δ ppm 7.12 (d, J=8.5 Hz, 1H), 5.33-5.26 (m, 2H), 5.05-4.94 (m, 2H), 4.36-4.19 (m, 4H), 3.99-3.87 (m, 3H), 3.62-3.25 (m, 5H), 3.21-2.85 (m, 4H), 2.31-2.07 (m, 6H), 2.06-2.01 (m, 3H), 1.96-1.86 (m, 1H), 0.87-0.72 (m, 4H).

### Example 13

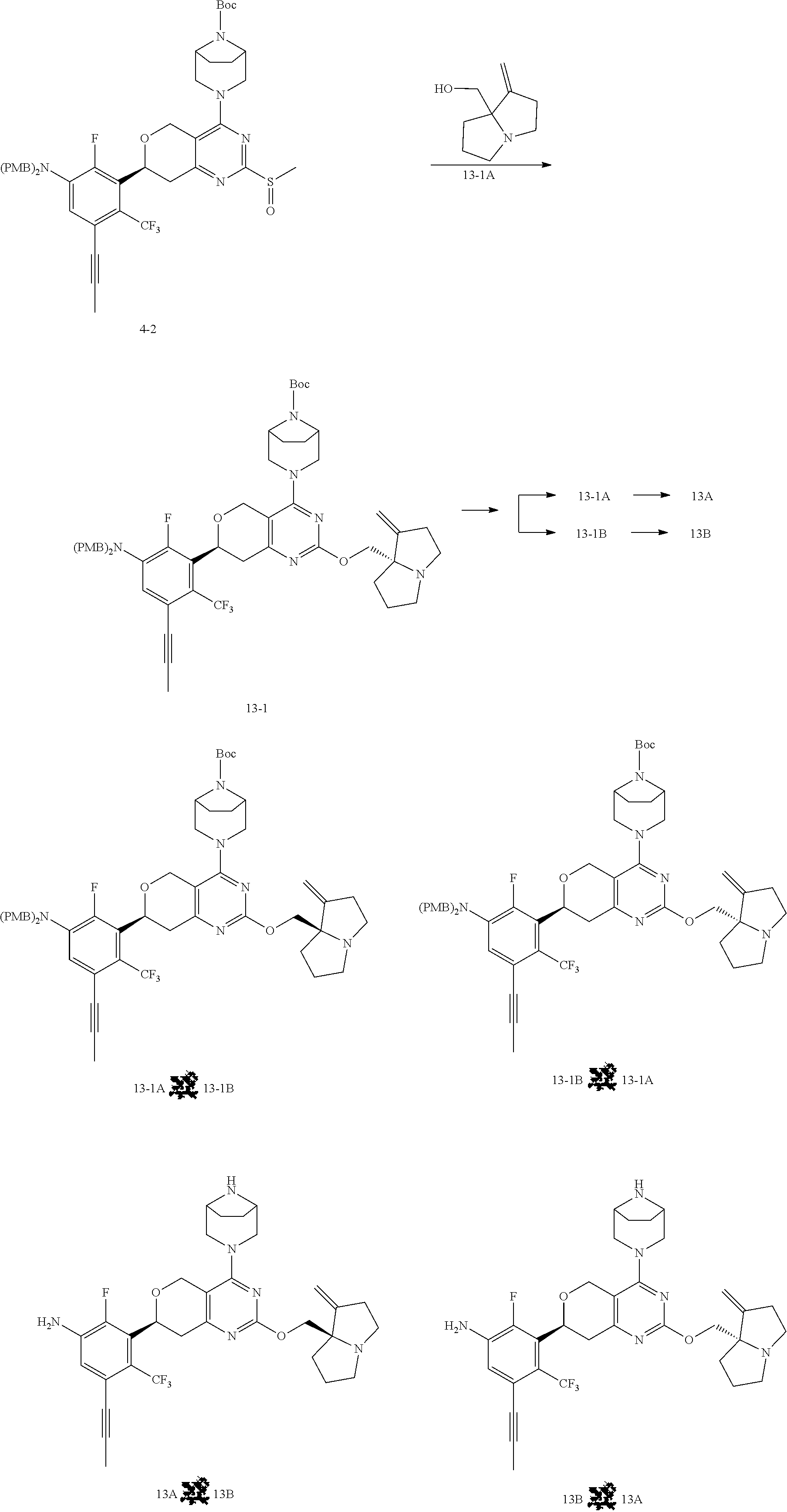

### Step 1

Compound 4-2 (50.0 mg, 57.9 μmol), compound 13-1A (26.6 mg, 174 μmol), sodium tert-butanol (27.8 mg, 289 μmol) and 4 Å molecular sieve (50.0 mg) were added to toluene (2.00 mL), and the reaction solution was reacted at 110° C. for 12 hours. The reaction solution was cooled to room temperature, filtered, and the filtrate was concentrated to obtain the crude product. The crude product was purified by column chromatography (dichloromethane:methanol=10:1) to obtain compound 13-1. MS m/z=953.5 [M+1]⁺. Compound 13-1 was prepared for SFC separation (chiral column: (s,s) WHELK-01 (250 mm*30 mm, 10 μm); mobile phase: [supercritical carbon dioxide-acetonitrile/isopropanol (0.1% ammonia water)]; acetonitrile/isopropanol (0.1% ammonia water): 38%-38%, and compound 13-1A and compound 13-1B were obtained after concentration under reduced pressure. Analyze SFC: (Chiral column: (s,s) WHELK-01 (50 mm*4.6 mm, 3.5 μm); mobile phase: [supercritical carbon dioxide-isopropanol (0.05% diethylamine)]; isopropanol (0.05% diethylamine) %. 40%, compound 13-1A, Rt=1.495 min, ee value 99%; MS m/z=953.5 [M+H]⁺; compound 13-1B, Rt=1.716 min, ee value 95%, MS m/z=953.5 [M+H]⁺.

### Step 2

Compound 13-1A (17.0 mg, 17.8 μmol) was dissolved in dichloromethane (1.00 mL), to which trifluoroacetic acid (0.30 mL) was added, and the reaction solution was reacted at 25° C. for 1 hour. The reaction solution was concentrated directly to obtain crude product. The crude product was prepared for high performance liquid chromatography separation (column: Phenomenex luna C18 150*25 mm*10 μm; mobile phase: [water (0.075% trifluoroacetic acid)-acetonitrile], acetonitrile. 10%-40% purification to obtain 13A of trifluoroacetate. MS m/z=613.4 [M+H]⁺, ¹H NMR (400 MHz, CD₃OD) δ ppm 6.92 (d, J=8.5 Hz, 1H), 5.41-5.27 (m, 2H), 5.18 (br dd, J=3.8, 10.8 Hz, 1H), 4.77-4.70 (m, 1H), 4.68-4.50 (m, 2H), 4.32 (br d, J=14.4 Hz, 1H), 4.22-4.10 (m, 2H), 3.88-3.63 (m, 4H), 3.48-3.32 (m, 4H), 3.27 (br d, J=12.1 Hz, 1H), 3.03-2.88 (m, 3H), 2.53-2.40 (m, 1H), 2.32-2.09 (m, 6H), 2.04-2.01 (m, 3H), 2.01-1.93 (m, 1H).

Compound 13-1B (20.0 mg, 21.0 μmol) was dissolved in dichloromethane (1.00 mL), to which trifluoroacetic acid (0.30 mL) was added, and the reaction solution was reacted at 25° C. for 1 hour. The reaction solution was concentrated directly to obtain crude product. The crude product was prepared for high performance liquid chromatography separation (column: Phenomenex luna C18 150*25 mm*10 μm; mobile phase: [water (0.075% trifluoroacetic acid)-acetonitrile]; acetonitrile: 10%-40% purification to obtain 13B of trifluoroacetate. MS m/z=613.5 [M+H]⁺, ¹H NMR (400 MHz, CD₃OD) δ ppm 6.94 (d, J=8.5 Hz, 1H), 5.44-5.30 (m, 2H), 5.20 (dd, J=3.8, 11.3 Hz, 1H), 4.79-4.73 (m, 1H), 4.70-4.56 (m, 2H), 4.39 (br d, J=14.0 Hz, 1H), 4.22-4.13 (m, 2H), 3.88-3.69 (m, 4H), 3.47-3.34 (m, 4H), 3.32-3.27 (m, 1H), 3.01-2.94 (m, 3H), 2.53-2.40 (m, 1H), 2.32-2.12 (m, 6H), 2.04 (s, 3H), 2.02-1.94 (m, 1H).

### Example 14

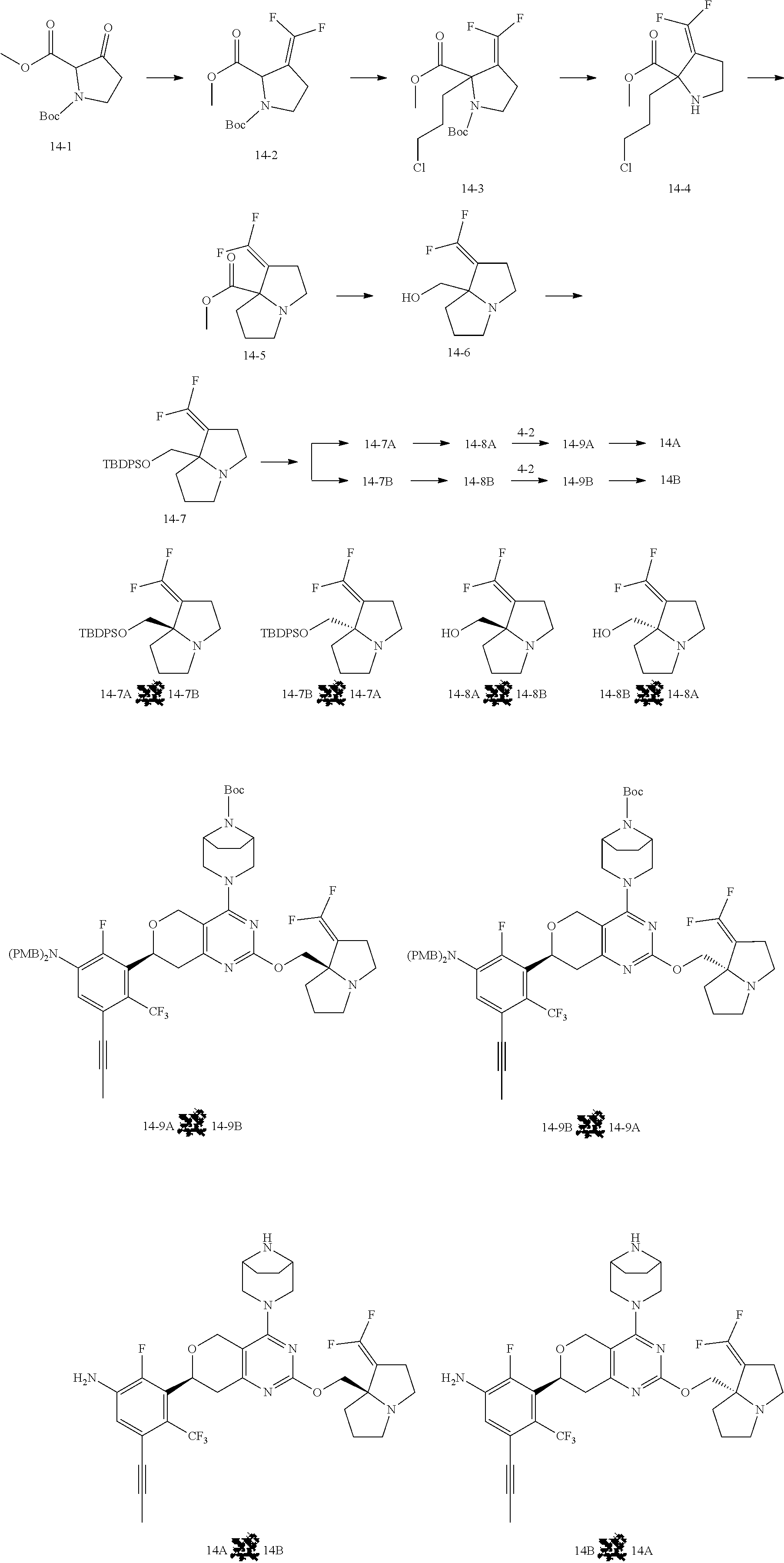

### Step 1

Compound 14-1 (5.00 g, 20.6 mmol) was dissolved in DMF (50.0 mL), then triphenylphosphoniumyl difluoroacetate (19.0 g, 53.4 mmol) was added and reacted at 80° C. for 2 hours. Add water (300 mL) to the reaction solution, and extract with ethyl acetate (10 mL) three times. The organic phase was washed with saturated brine (200 mL), dried over anhydrous sodium sulfate, filtered, and concentrated to obtain the crude product. The crude product was purified by column chromatography (petroleum ether:ethyl acetate=100:1-3:1) to obtain compound 14-2. ¹H NMR (400 MHZ, CDCl₃) 5.00-4.76 (m, 1H), 3.82-3.65 (m, 4H), 3.61-3.45 (m, 1H), 2.71-2.55 (m, 2H), 1.49-1.38 (m, 9H).

### Step 2

Compound 14-2 (2.80. g, 10.1 mmol) was dissolved in tetrahydrofuran (3.00 mL), lithium diisopropylamide (2.00 M, 10.1 mL) was added, 1-chloro-3-iodopropane (10.3 g, 50.5 mmol, 1.55 mL) was added after reaction at −60° C. for 1 hour, then 1 hours at −60° C., and 12 hours at 25° C. To the reaction solution was added 50.0 mL of ammonium chloride solution and extracted 3 times with ethyl acetate (50.0 mL). The organic phase was washed with saturated brine (100 mL), dried over anhydrous sodium sulfate, filtered, and concentrated to obtain the crude product. The crude product was purified by column chromatography (petroleum ether:ethyl acetate=50:1-10:1) to obtain compound 14-3. MS m/z=376.1 [M+Na]⁺.

### Step 3

Compound 14-3 (3.20 g, 9.04 mmol) was dissolved in acetonitrile (1.00 mL), hydrochloric acid/dioxane (2.00 M, 5.00 mL) was added and reacted at 25° C. for 12 hours. The solvent was removed by concentration under reduced pressure to obtain the hydrochloride salt of compound 14-4. MS m/z=254.1 [M+H]⁺.

### Step 4

Compound 14-4 (2.50 g, hydrochloride) was dissolved in acetonitrile (25.0 mL), potassium carbonate (5.95 g, 43.1 mmol) and potassium iodide (143 mg, 862 μmol) were added, and the reaction was performed at 25° C. for 12 hours, filtered, concentrated to obtain the crude product. The crude product was purified by column chromatography (petroleum ether:ethyl acetate=50:1-20:1) to obtain compound 14-5. MS m/z=218 [M+H]⁺.

### Step 5

Compound 14-5 (300 mg, 1.38 mmol) was dissolved in THF (3.00 mL), lithium aluminum hydride (2.50 M, 1.10 mL) was added, and then reacted at 0° C. for 1 hour. 0.11 mL of water, 0.11 mL of 15% sodium hydroxide solution, and 0.33 mL of water were added into the reaction solution, filtered, concentrated to obtain compound 14-6. MS m/z=190.1 [M+H]⁺.

### Step 6

Compounds 14-6 (300 mg, 1.59 mmol) were dissolved in anhydrous methylene chloride (3.00 mL), imidazole (432 mg, 6.34 mmol), 4-dimethylaminopyridine (19.37 mg, 159 μmol) and tert-butylchlorodiphenylsilane (872 mg, 3.17 mmol, 812 μL) were added and reacted at 25° C. for 12 hours. The reaction solution was concentrated to obtain the crude product. The crude product was prepared for high performance liquid chromatography separation (column: Phenomenex luna C18 150*25 mm*10 μm; mobile phase: [water (0.075% trifluoroacetic acid)-acetonitrile]; acetonitrile: 25%-55%) to obtain compound 14-7. MS m/z=428.3 [M+H]⁺. Compound 14-7 was prepared for SFC separation (chiral column: DAICEL CHIRALCEL OX (250 mm*50 mm, 10 μm); mobile phase: [supercritical carbon dioxide-methanol (0.1% ammonia)]; methanol (0.1% ammonia) is 10%-10%. After concentration under reduced pressure, compound 14-7A and compound 14-7B were obtained. Analyze SFC: (Chiral column: DAICEL CHIRALCEL OX (50 mm*4.6 mm, 3 μm); mobile phase: [supercritical carbon dioxide-methanol (0.05% diethylamine)]; methanol (0.05% diethylamine)]; methanol (0.05% diethylamine): 5%-40%, compound 14-7A, Rt=1.165 min, ee value 99%, MS m/z=428.2 [M+H]⁺; compound 14-7B, Rt=1.233 min, ee value 95% MS m/z=428.2 [M+H]⁺.

### Step 7

Compounds 14-7A (126 mg, 295 μmol) were dissolved in dioxane (2.00 mL), hydrochloric acid (12 M, 0.50 mL) was added, and reacted at 95° C. for 12 hours. Add 2 mL of water to dissolve, extract with ethyl acetate (10 mL), and obtain the hydrochloride salt of compound 14-8A from the lyophilized aqueous phase. MS m/z=190.1 [M+H]⁺.

With reference to step 7, compound 14-7A was replaced with compound 14-7B as the raw material to obtain the hydrochloride salt of compound 14-8B. MS m/z=190.1 [M+H]⁺.

### Step 8

Compound 4-2 (120 mg, 139 μmol), compound 14-8A (62.7 mg, hydrochloride), sodium tert-butanol (66.7 mg, 695 μmol) and 4 Å molecular sieve (30.0 mg) were added to toluene (3.00 mL), and the reaction solution was reacted at 100° C. for 6 hours. The reaction solution was cooled to room temperature, filtered, and the filtrate was concentrated to obtain the crude product. The crude product was purified by thin layer chromatography (pure ethyl acetate) to obtain compound 14-9A. MS m/z=989.7 [M+1]⁺.

With reference to step 8, compound 14-8A was replaced with compound 14-8B as the raw material to obtain compound 14-9B. MS m/z=989.6 [M+H]⁺.

### Step 9

Compound 14-9A (63.0 mg, 63.7 μmol) was dissolved in dichloromethane (4.00 mL), to which trifluoroacetic acid (1.00 mL) was added, and the reaction solution was reacted at 25° C. for 12 hours. The reaction solution was concentrated directly to obtain crude product. Preparation of crude product for high performance liquid chromatography separation (column: Phenomenex luna C18 150*25 mm*10 μm; mobile phase: [water (0.075% trifluoroacetic acid)-acetonitrile]; acetonitrile: 16%-46% purification to obtain 14A of trifluoroacetate. MS m/z=649.2 [M+H], 1H NMR (400 MHz, MeOD) δ ppm 6.92 (d, J=8.5 Hz, 1H), 5.19 (br dd, J=3.9, 11.1 Hz, 1H), 4.96-4.90 (m, 1H), 4.84-4.78 (m, 1H), 4.77-4.70 (m, 2H), 4.66-4.58 (m, 1H), 4.32 (br d, J=14.0 Hz, 1H), 4.15 (br dd, J=3.0, 13.6 Hz, 2H), 3.97-3.63 (m, 4H), 3.57-3.39 (m, 2H), 3.36-3.33 (m, 1H), 3.09-2.87 (m, 3H), 2.49-2.05 (m, 7H), 2.04-1.93 (m, 4H).

Compound 14-9B (88.0 mg, 89.0 μmol) was dissolved in dichloromethane (4.00 mL), to which trifluoroacetic acid (1.00 mL) was added, and the reaction solution was reacted at 25° C. for 12 hours. The reaction solution was concentrated directly to obtain crude product. Preparation of crude product for high performance liquid chromatography separation (column: Phenomenex luna C18 150*25 mm*10 μm; mobile phase: [water (0.075% trifluoroacetic acid)-acetonitrile], acetonitrile. 16%-46% purification to obtain 14B of trifluoroacetate. MS m/z=649.2 [M+H]⁺, ¹H NMR (400 MHz, MeOD) δ ppm 6.92 (d, J=8.5 Hz, 1H), 5.19 (br dd, J=4.1, 11.3 Hz, 1H), 4.93-4.88 (m, 2H), 4.80-4.70 (m, 2H), 4.59 (d, J=12.4 Hz, 1H), 4.30 (br d, J=14.1 Hz, 1H), 4.15 (br dd, J=2.1, 14.3 Hz, 2H), 3.95-3.62 (m, 4H), 3.58-3.38 (m, 2H), 3.30-3.24 (m, 1H), 3.09-2.87 (m, 3H), 2.47-2.07 (m, 7H), 2.05-1.94 (m, 4H).

### Example 15

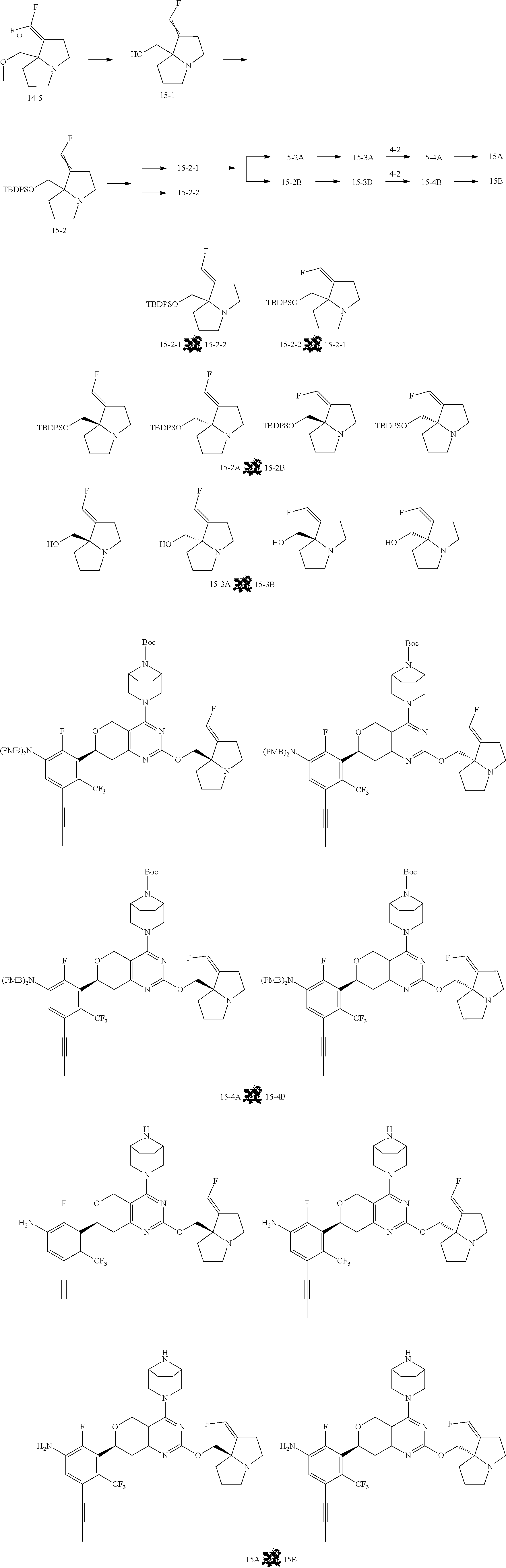

### Step 1

Compounds 14-5 (500 mg, 2.30 mmol) were dissolved in 2-MeTHF (5.00 mL), bis(2-methoxyethoxy)aluminum sodium hydride (2.66 g, 9.21 mmol, 2.57 mL, 70% purity) was added and reacted at 10° C. for 2.5 hours. Then it was reacted at room temperature for 12 hours, 10.0 mL of water was added to the reaction solution, concentrated to remove dimethyltetrahydrofuran, and lyophilized to obtain compound 15-1. MS m/z=172.1 [M+H]⁺.

### Step 2

Compound 15-1 (261 mg, 1.52 mmol) was dissolved in anhydrous methylene chloride (3.00 mL), imidazole (415 mg, 6.10 mmol), 4-dimethylaminopyridine (18.6 mg, 152 μmol) and tert-butylchlorodiphenylsilane (838 mg, 3.05 mmol, 780 μL) were added and reacted at 45° C. for 12 hours. The reaction solution was concentrated to obtain the crude product. Crude product was performed with the preparation of high performance liquid chromatography separation (column: Phenomenex luna C18 150*25 mm*10 μm; mobile phase: [water (0.075% trifluoroacetic acid)-acetonitrile]; acetonitrile: 35%-65% to obtain compound 15-2, MS m/z=410.3 [M+H]⁺. Compound 15-2 was prepared for SFC separation (chiral column: DAICEL CHIRALPAK IG (250 mm*50 mm, 10 μm); mobile phase: [supercritical dichloromethane-methanol (0.1% ammonia water]); methanol (0.1% ammonia water): 18%-18%); after concentration under reduced pressure, compound 15-2-1 and compound 15-2-2 were obtained. Analyze SFC: (Chiral column: Chiralcel OX-3 (50 mm*4.6 mm, 3 μm); mobile phase: [supercritical carbon dioxide-ethanol (0.05% diethylamine]); ethanol (0.05% diethylamine) %: 5%-40%, compound 15-2-1, Rt=1.18 min, ee value 99%; MS m/z=410.2 [M+H]; compound 15-2-2, Rt=1.46 min, ee value 97% MS m/z=[410.2M+H]+.

Chiral preparation HPLC separation of compound 15-2-1 was performed (chiral column: DAICEL CHIRALCEL OX (250 mm*50 mm, 10 μm); mobile phase: [n-hexane-ethanol (0.1% ammonia water)]; ethanol (0.1% ammonia water, 10%-10%. After concentration under reduced pressure, compound 15-2A and compound 15-2B were obtained Analyze SFC: (Chiral column: Chiralcel OX-3 (50 mm*4.6 mm, 3 μm); mobile phase: [supercritical carbon dioxide-ethanol (0.05% diethylamine]); ethanol (0.05% diethylamine) %: 5%-40%, compound 15-2A, Rt=1.18 min, ee value 99%; MS m/z=410.2 [M+H]⁺; compound 15-2B, Rt=1.21 min, ee value 99% MS m/z=410.2 [M+H]⁺.

### Step 3

Compound 15-2A (160 mg, 391 μmol) was dissolved in dioxane (4.00 mL), hydrochloric acid (12 M, 1.00 mL) was added and reacted at 95° C. for 12 hours. Add 5 mL of water to dissolve, extract with ethyl acetate (3.0 mL), and obtain the hydrochloride salt of compound 15-3A from the lyophilized aqueous phase. MS m/z=172.1 [M+H]⁺.

With reference to step 3, compound 15-2B was replaced with compound 15-2A as the raw material to obtain the hydrochloride salt of compound 15-3B. MS m/z=172.1 [M+H]⁺.

### Step 4

Compound 4-2 (100 mg, 116 μmol), compound 15-3A (48.1 mg, hydrochloride), sodium tert-butanol (55.6 mg, 579 μmol) and 4 Å molecular sieve (50.0 mg) were added to toluene (3.00 mL), and the reaction solution was reacted at 100° C. for 6 hours. The reaction solution was cooled to room temperature, filtered, and the filtrate was concentrated to obtain the crude product. The crude product was purified by preparing a thin layer of chromatography separation (pure ethyl acetate) to obtain compound 15-4A. MS m/z=971.4 [M+1]⁺.

With reference to Step 4, the hydrochloride of compound 15-3B was used as the raw material to replace the hydrochloride of Compound 15-3A to obtain compound 15-4B. MS m/z=971.4 [M+H]⁺.

### Step 5

Compound 15-4A (71.0 mg, 73.1 μmol) was dissolved in dichloromethane (5.00 mL), to which trifluoroacetic acid (1.00 mL) was added, and the reaction solution was reacted at 25° C. for 12 hours. The reaction solution was concentrated directly to obtain crude product. The crude product was prepared for high performance liquid chromatography separation (column: Waters Xbridge 150*25 mm*5 μm; mobile phase: [water (0.1% ammonia)-acetonitrile]; acetonitrile: 35%-65% purification to obtain compound 15A. MS m/z=631.3 [M+H]⁺, ¹H NMR (400 MHZ, MeOD) δ ppm 6.99-6.71 (m, 2H), 5.14 (br dd, J=4.1, 11.5 Hz, 1H), 4.81-4.76 (m, 2H), 4.20-4.09 (m, 3H), 3.61-3.38 (m, 4H), 3.26-3.00 (m, 4H), 2.90-2.58 (m, 5H), 2.25-2.10 (m, 1H), 2.07-1.60 (m, 10H). Compound 15-4B (61.0 mg, 62.8 μmol) was dissolved in dichloromethane (5.00 mL), to which trifluoroacetic acid (1.00 mL) was added, and the reaction solution was reacted at 25° C. for 12 hours. The reaction solution was concentrated directly to obtain crude product. The crude product was prepared for high performance liquid chromatography separation (column: Phenomenex luna C18 150*25 mm*10 μm; mobile phase: [water (0.075% trifluoroacetic acid)-acetonitrile]; acetonitrile: 10%-40% purification to obtain 15B of trifluoroacetate. MS m/z=631.3 [M+H]⁺, ¹H NMR (400 MHz, MeOD) δ ppm 7.27-6.84 (m, 2H), 5.19 (br dd, J=3.5, 11.0 Hz, 1H), 4.79-4.50 (m, 4H), 4.34 (br d, J=14.3 Hz, 1H), 4.15 (br dd, J=2.9, 14.6 Hz, 2H), 3.91-3.61 (m, 4H), 3.49-3.33 (m, 3H), 3.30-3.24 (m, 1H), 3.17-2.87 (m, 3H), 2.54-2.05 (m, 7H), 2.04-1.92 (m, 4H).

### Example 16

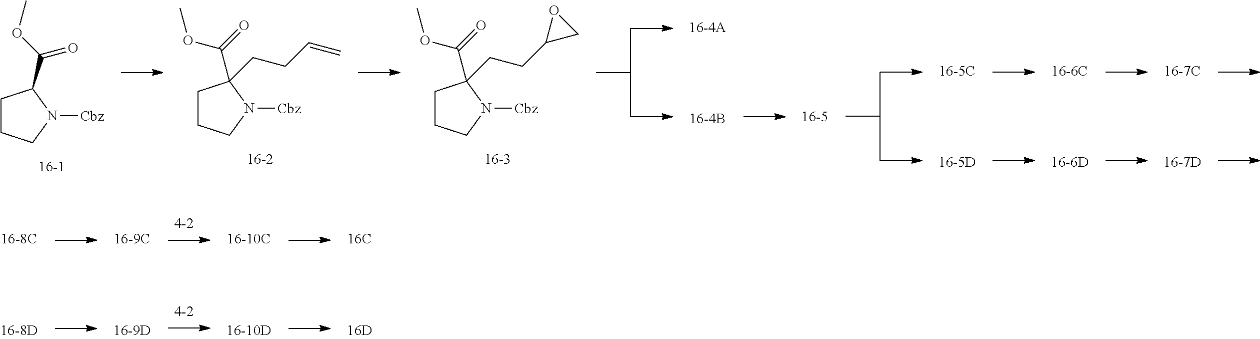 [SMILES: COC(=O)[C@@H]1CCCN1C(=O)OCc1ccccc1 ; C=CCCC1(C(=O)OC)CCCN1C(=O)OCc1ccccc1 ; COC(=O)C1(CCC2CO2)CCCN1C(=O)OCc1ccccc1]

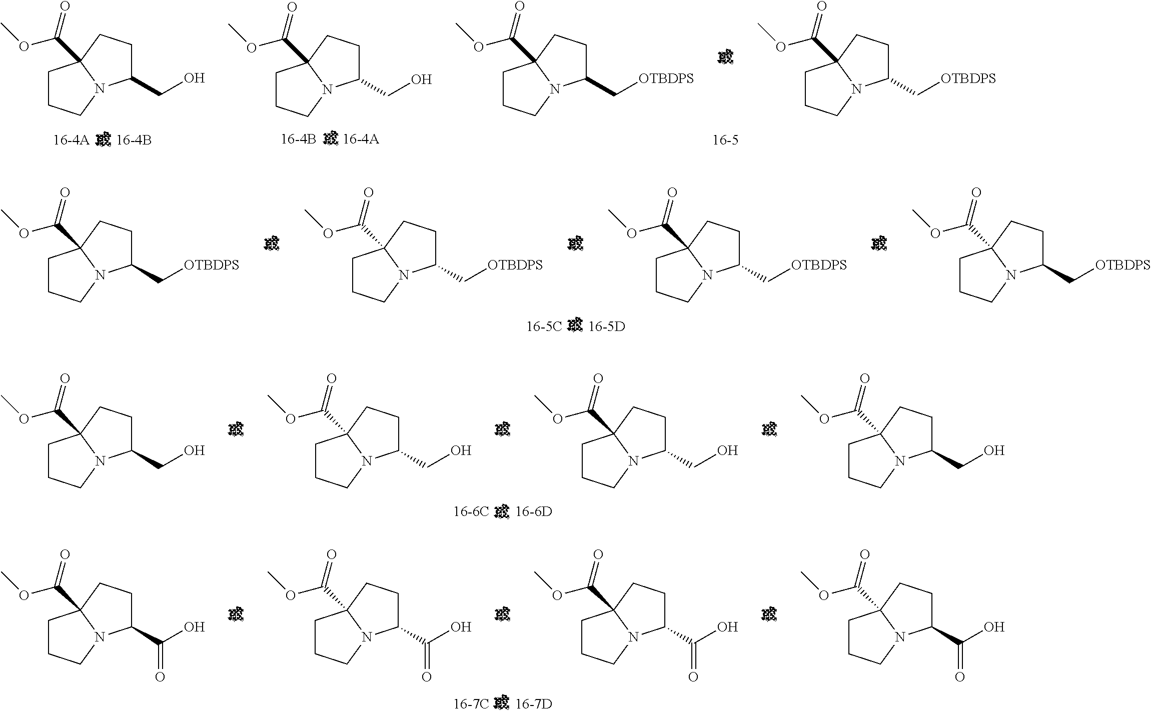

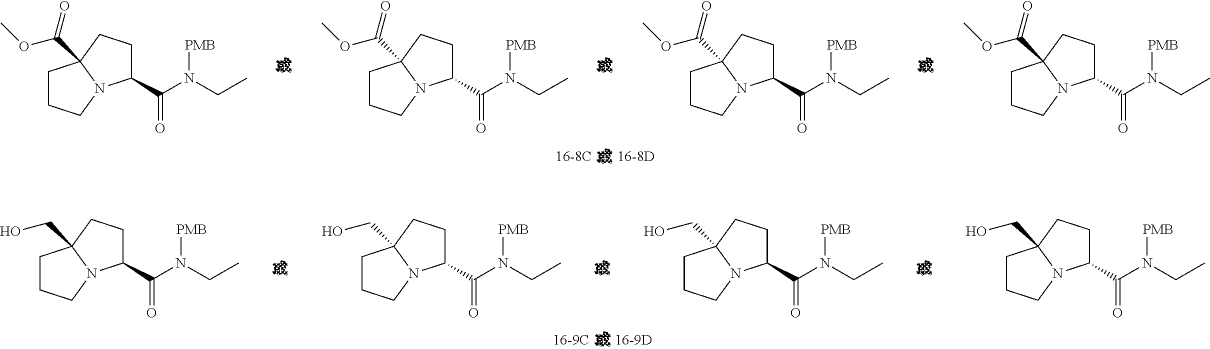

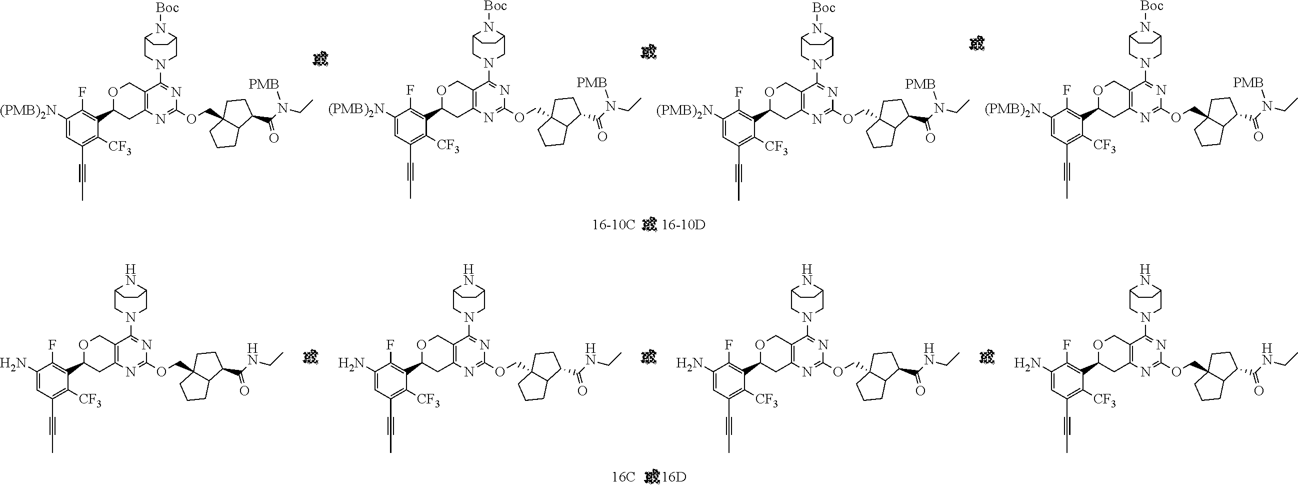

### Step 1

The solution of tetrahydrofuran (569.72 mL, 569.72 mmol, 1M) of ditrimethylsilylaminolithium was slowly added dropwise to the solution of compound 16-1 (100.00 g, 379.81 mmol) in tetrahydrofuran (1000 mL) at −70° C. for 1 hour. 4-bromo-1-butene (128.19 g, 949.53 mmol) was added dropwise to the reaction solution at −70° C., and the reaction was stirred at 20° C. for 12 hours. Added 1000 mL of saturated aqueous ammonium chloride solution for quench reaction, extracted with ethyl acetate (700 mL*3), dried with anhydrous sodium sulfate, filtered, concentrated under reduced pressure, separated for column chromatography (petroleum ether:ethyl acetate=9:1) to yielded compound 16-2, MS m/z=318.0 [M+H]⁺.

### Step 2

Compound 16-2 (96.00 g, 302.48 mmol) was dissolved in dichloromethane (1,000 mL), metachloroperoxybenzoic acid (135.10 g, 665.45 mmol, 85% purity) was added, and the reaction was stirred and reacted at 20° C. for 12 hours under nitrogen protection. The reaction was quenched by adding saturated sodium sulphite aqueous solution (1500 mL), washed with saturated sodium bicarbonate aqueous solution (1000 mL), the organic phase separated and dried with anhydrous sodium sulfate, filtered, concentrated under reduced pressure, and column chromatography separated (petroleum ether:ethyl acetate=6:1) to obtain compound 16-3, MS m/z=334.1 [M+H]⁺.

### Step 3

For solution of compound 16-3 (79.00 g, 236.97 mmol) in methanol (1,500 mL), palladium/carbon (16.64 g, 156.40 mmol, 10% purity) was added and reacted at 20° C. for 16 hours in a hydrogen gas (15 psi). The filtrate was filtered and concentrated under reduced pressure, and column chromatography separation was performed (dichloromethane:methanol=20:1 to 9:1) to obtain compound 16-4A (drilling agent: dichloromethane:methanol=15:1, Rf=0.5) and compound 16-4B (drilling agent: dichloromethane:methanol=15:1, Rf=0.3), respectively. Compound 16-4A: MS m/z=200.0 [M+H]⁺. ¹H NMR (400 MHZ, CDCl₃) δ ppm 3.71 (s, 3H), 3.60 (dd, J=12.0 Hz, 4.0 Hz, 1H), 3.41 (dd, J=12.0 Hz, 4.0 Hz, 1H), 3.13-3.05 (m, 1H), 2.99-2.94 (m, 1H), 2.73-2.67 (m, 1H), 2.36-2.31 (m, 1H), 2.24-2.20 (m, 1H), 1.93-1.79 (m, 5H), 1.74-1.66 (m, 1H). Compounds 16-4B: MS m/z=200.0 [M+H]⁺. ¹H NMR (400 MHZ, CDCl₃) δ ppm 3.90 (dd, J=12.0 Hz, 8.0 Hz, 1H), 3.79 (dd, J=12.0 Hz, 5.2 Hz, 1H), 3.73 (s, 3H), 3.49-3.42 (m, 1H), 3.11-3.07 (m, 1H), 2.76-2.69 (m, 1H), 2.58-2.52 (m, 1H), 2.29-2.21 (m, 1H), 1.89-1.80 (m, 4H), 1.67-1.54 (m, 2H).

### Step 4

Compound 16-4B (21.00 g, 105.40 mmol) was dissolved in dichloromethane (200 mL), imidazole (15.07 g, 221.33 mmol) was added at 0° C., tert-butylchlorodiphenylsilane (37.66 g, 137.02 mmol) was added after stirring for 10 minutes, and the temperature was increased to 20° C. for stirring for 7 hours. Filtration, concentration of filtrate under reduced pressure, column chromatography separation (petroleum ether: Ethyl acetate=8:1) to obtain compound 16-5, MS m/z=438.0 [M+H]⁺. The chiral preparation HPLC separation (chromatographic column: Regis: S,S) Whelk-O 1, 25*250 mm 10 μm; mobile phase: A: n-hexane, B: ethanol; B %: 2% to obtain compound 16-5C and compound 16-5D. SFC analytical method (column: Regis (s,s) WHELK-01 (4.6 mmI.D*150 mmL, 5 μm); mobile phase: [supercritical carbon dioxide-methanol (0.05% diethylamine]; gradient: methanol (0.05% diethylamine) %: 5%-40%, 4 min), compound 16-5C, Rt=3.640 min, ee value 97.06%, MS m/z=438.0 [M+H]⁺; compound 16-5D, Rt=3.826 min, ee value 97.88%, MS m/z=438.0 [H]⁺.

### Step 5

Compound 16-5C (12.00 g, 27.42 mmol) was dissolved in hydrogen chloride/1, 4-dioxane solution (100 mL, 4M), and the reaction was stirred at 50° C. for 16 hours. The reaction solution was concentrated under reduced pressure, and column chromatography separated (dichloromethane:methanol=7:1) to obtain compound 16-6C. MS m/z=200.0 [M+H]⁺.

With reference to step 5, compound 16-5C was replaced with compound 16-5D as the raw material to obtain compound 16-6D. MS m/z=200.0 [M+H]⁺.

### Step 6

Compound 16-6C (1.00 g, 5.02 mmol) was dissolved in 10 mL of mixed solvent (acetonitrile:water=100:0.75), chromium trioxide (150.56 mg 1.51 mmol) and periodic acid (2.86 g, 12.55 mmol) were added at 0° C., and the solution was stirred under nitrogen protection for 16 hours. The reaction solution was filtered, and the filtrate was concentrated under reduced pressure to obtain compound 16-7C. MS m/z=214.0 [M+H]⁺.

With reference to step 6, compound 16-6C was replaced with compound 16-6D as the raw material to obtain compound 16-7D. MS m/z=214.0 [M+H]⁺.

### Step 7

Compound 16-7C (0.20 g, 0.94 mmol) was dissolved in N,N dimethylformamide (5 mL), followed by addition of N-ethyl-4-methoxybenzylamine (154.98 mg, 0.94 mmol), N,N-diisopropylethylamine (606.12 mg. 4.69 mmol 2-(7-azobenzotriazazole)-N,N,N′,N′-tetramethylurea hexafluorophosphate phosphate (713.28, 1.88 mmol), and reaction was stirred at room temperature for 1 hour. Add water (50 mL) to the reaction solution, extract with ethyl acetate (20 mL*3), combine the organic phases, wash with saturated brine (50 mL), dry with anhydrous sodium sulfate, filter, concentrate under reduced pressure, and separate by column chromatography (petroleum ether:ethyl acetate=2:1 to obtain compound 16-8C. MS m/z=361.0 [M+H]⁺.

With reference to step 7, compound 16-7C was replaced with compound 16-7D as the raw material to obtain compound 16-8D MS m/z=361.0 [M+H]⁺.

### Step 8

Compounds 16-8C (150 mg, 0.42 mmol) were dissolved in anhydrous methanol (3 mL), cooled to 0° C., followed by sodium borohydride (50 mg, 1.25 mmol in sodium methoxide/methanol solution (7.49 mg, 41.62 μmol, 30% purity), and the reaction was stirred at room temperature for 8 hours. The reaction was quenched by adding saturated aqueous solution (10 mL) of ammonium chloride, extracted with ethyl acetate (20 mL*3), combined with organic phase, washed with saturated brine (20 mL), dried with anhydrous sodium sulfate, filtered, concentrated under reduced pressure, and separated by column chromatography (petroleum ether:ethyl acetate=1:1) to obtain compound 16-9C. MS m/z=333.3 [M+H]⁺.

With reference to step 8, compound 16-8C was replaced with compound 16-8D as the raw material to obtain compound 16-9D MS m/z=333.3 [M+H]⁺.

### Step 9

Compound 16-9C (0.10 g, 0.30 mmol) was dissolved in anhydrous tetrahydrofuran (3 mL), cooled to 0° C., sodium hydrogen (60.16 mg, 1.50 mmol, 60% purity) was added, and compound 4-2 (264.70 mg, 0.30 mmol) was added after stirring at 0° C. for half an hour, and the reaction was stirred at room temperature for 1 hour. Add saturated aqueous solution of ammonium chloride (10 mL) and quench, extract with ethyl acetate (20 mL*3), combine the extracted organic phases, wash with saturated esophagus saline (20 mL), dry with anhydrous sodium sulfate, filter, concentrate under reduced pressure, and perform column chromatography separation (petroleum ether:ethyl acetate=2:1) to obtain compound 16-10C. MS m/z=1132.5 [M+H]⁺.

With reference to step 9, compound 16-9C was replaced with compound 16-9D as the raw material to obtain compound 16-10D. MS m/z=1132.5 [M+H]⁺.

### Step 11

Compound 16-10C (0.20 g, 0.18 mmol) was added to trifluoroacetic acid (4 mL), and the solution was stirred and reacted at 60° C. for 1 hour. The reaction solution was concentrated under reduced pressure to obtain crude product. Crude product was prepared for high performance liquid chromatography separation (chromatographic column: Waters Xbridge, 250*19 mm, 5 mm; mobile phase: [water (0.1% ammonia water)-acetonitrile]; gradient: (acetonitrile): 55%-70%, lyophilized to obtain compound 16C. MS m/z=672.5 [M+H]⁺. 1H NMR (400 MHZ, CD₃OD) δ ppm 6.90 (d, J=8.0 Hz, 1H), 5.16-5.14 (m, 1H), 4.81-4.78 (m, 1H), 4.67-4.63 (m, 1H), 4.16-4.13 (m, 2H), 4.09-4.08 (m, 1H), 3.75-3.72 (m, 1H), 3.54-3.51 (m, 3H), 3.43-3.40 (m, 1H), 3.28-3.17 (m, 3H), 3.06-3.03 (m, 1H), 2.91-2.83 (m, 2H), 2.78-2.72 (m, 1H), 2.12-2.08 (m, 2H), 2.02-1.91 (m, 11H), 1.76-1.66 (m, 2H), 1.14 (t, J=8.0 Hz, 3H).

Compound 16-10D (0.16 g, 0.14 mmol) was added to trifluoroacetic acid (4 mL), and the reaction was stirred and reacted at 60° C. for 1 hour. The reaction solution was concentrated under reduced pressure to obtain crude product. Crude product was prepared for high performance liquid chromatography separation (chromatographic column: Waters Xbridge, 250*19 mm, 5 mm; mobile phase: [water (0.1% ammonia water)-acetonitrile]; gradient: (acetonitrile): 55%-70%, lyophilized to obtain compound 16D. MS m/z=672.5 [M+H]⁺. ¹H NMR (400 MHZ, CD₃OD) δ ppm 6.90 (d, J=8.0 Hz, 1H), 5.17-5.12 (m, 1H), 4.84-4.78 (m, 1H), 4.66-4.63 (m, 1H), 4.18-4.12 (m, 2H), 4.05-4.03 (m, 1H), 3.74-3.71 (m, 1H), 3.53-3.50 (m, 3H), 3.43-3.40 (m, 1H), 3.28-3.17 (m, 3H), 3.05-3.02 (m, 1H), 2.92-2.82 (m, 2H), 2.78-2.71 (m, 1H), 2.13-2.07 (m, 2H), 2.01-1.82 (m, 11H), 1.76-1.66 (m, 2H), 1.14 (t, J=8.0 Hz, 3H).

### Example 17

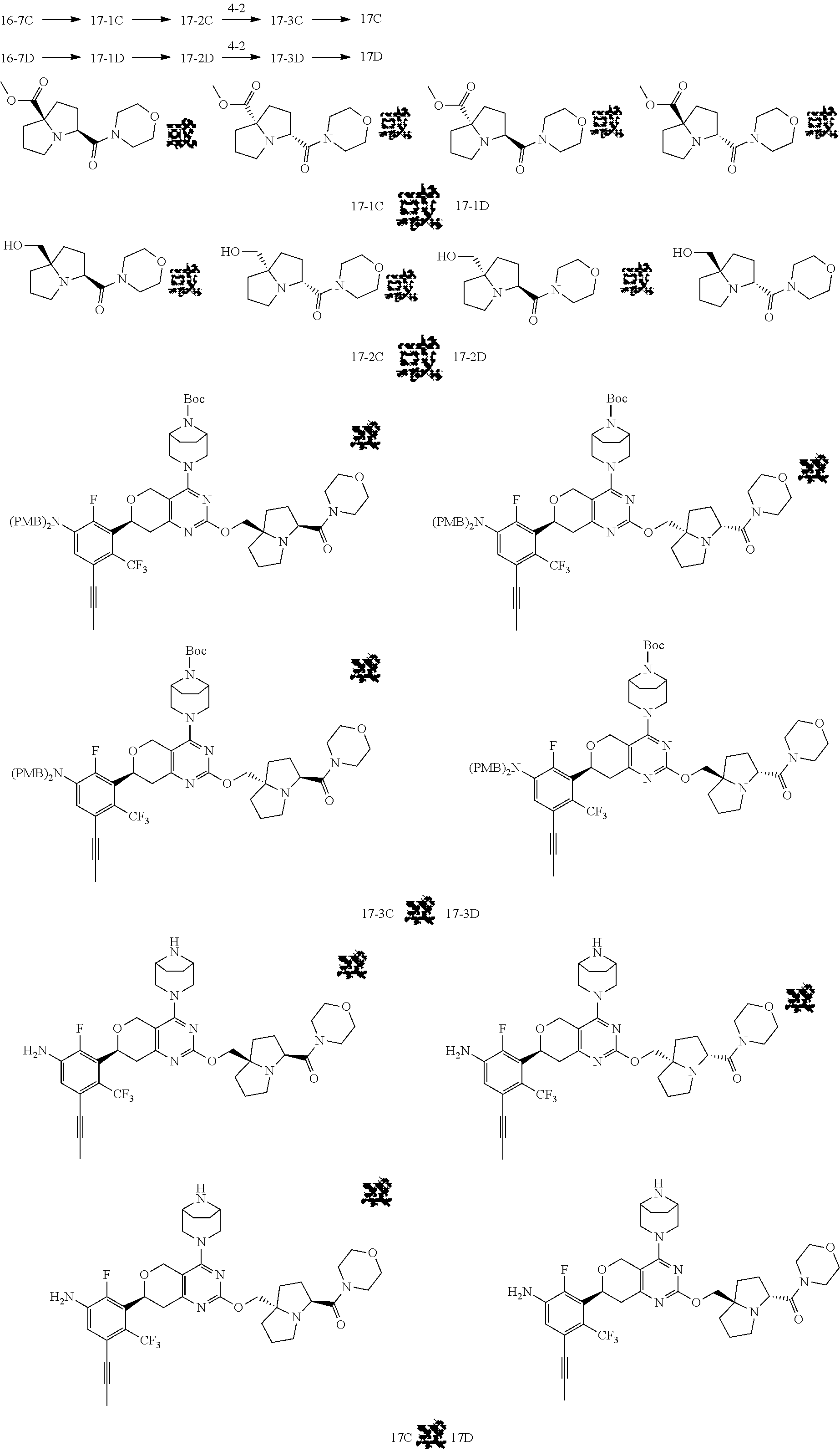

### Step 1

Compound 16-7C (0.90 g, 4.22 mmol) was dissolved in N, N-dimethylformamide (10 mL) followed by addition of N, N-diisopropylethylamine (2.73 g, 21.10 mmol), N,N,N′,N′-tetramethyl-O-(7-azabenzotriazole-1-yl) hexafluorourea phosphate (3.21 g, 8.44 mmol), morpholine 735.43 mg, 8.44 mmol) and reaction at 25° C. for 12 hours. The reaction solution was added with water (10 mL), extracted with ethyl acetate (20 mL*3), combined with the extracted organic phase, washed with saturated brine (10 mL), dried with anhydrous sodium sulfate, filtered, concentrated under reduced pressure, and column chromatography separated (dichloromethane:methanol=20:1) to obtain compound 17-1C. MS m/z=283.2 [M+H]⁺.

With reference to step 1, compound 16-7C was replaced with compound 16-7D as the raw material to obtain compound 17-1D. MS m/z=283.2 [M+H]⁺.

### Step 2

Compound 17-1C (20.00 mg, 70.84 μmol) was dissolved in methanol (0.5 mL), sodium methoxide (38.27 μg, 0.71 μmol) was added and sodium borohydride (8.04 mg, 212.51 μmol) at 0° C., and reacted at 25° C. for 16 hours. The reaction was quenched by adding saturated ammonium chloride (10 mL), extracted with ethyl acetate (20 mL), dried with anhydrous sodium sulfate, filtered, and concentrated under reduced pressure to obtain compound 17-2C. MS m/z=255.3 [M+H]⁺.

With reference to step 2, compound 17-1C was replaced with compound 17-1D as the raw material to obtain compound 17-2D. MS m/z=255.3 [M+H]⁺

### Step 3

Compound 17-2C (66.00 mg, 259.51 μmol) was dissolved in tetrahydrofuran (10 mL), sodium hydrogen (31.14 mg, 1.30 mmol, 60% purity) was added at 0° C., stirred for 0.5 hours, and compound 4-2 (228.36 mg, 259.51 μmol) was added at the end, and reacted at 25° C. for 2 hours. Add water (10 mL) to quench the reaction, extract with ethyl acetate (20 mL), dry with anhydrous sodium sulfate, filter, concentrate under reduced pressure, and separate by column chromatography (petroleum ether: ethyl acetate=0:1) to obtain compound 17-3C. MS m/z=1054.9 [M+H]⁺.

With reference to step 3, compound 17-2C was replaced with compound 17-2D as the raw material to obtain compound 17-3D. MS m/z=1054.9 [M+H]⁺.

### Step 4

Compound 17-3C (0.20 g, 189.72 μmol) was dissolved in dichloromethane (4 mL), trifluoroacetic acid (2 mL) was added, and reacted at 25° C. for 0.5 hour. The reaction solution was concentrated under reduced pressure to obtain crude product. The crude product was prepared for high performance liquid chromatography separation (chromatographic column: Waters SunFire, 250*19 mm, 5 μm; mobile phase: [water (0.1% formic acid)-acetonitrile]; gradient: (acetonitrile): 40%-75%. After lyophilization, the formate of compound 17C was obtained. MS m/z=714.6 [M+H]⁺, ¹H NMR (400 MHz, CD₃OD) δ ppm 8.29 (s, 1H), 6.91 (d, J=8.0 Hz, 1H), 5.18 (dd, J=8.0, 4.0 Hz, 1H), 4.82 (s, 1H), 4.71 (d, J=16.0 Hz, 1H), 4.30-4.22 (m, 3H), 4.12 (d, J=12.0 Hz, 2H), 4.04 (dd, J=12.0, 4.0 Hz, 1H), 3.78-3.57 (m, 9H), 3.52-3.46 (m, 1H), 3.25 (d, J=12.0 Hz, 2H), 2.94-2.87 (m, 2H), 2.76-2.70 (m, 1H), 2.31-2.24 (m, 2H), 2.12-2.09 (m, 3H), 2.02 (s, 3H), 1.99-1.83 (m, 6H), 1.74-1.66 (m, 1H).

Compound 17-3D (0.10 g, 94.86 μmol) was dissolved in dichloromethane (2 mL), trifluoroacetic acid (1 mL) was added, and reacted at 25° C. for 0.5 hour. The reaction solution was concentrated under reduced pressure to obtain crude product. Crude product was prepared for high performance liquid chromatography separation (chromatographic column: Waters Xbridge, 250*19 mm, 5 mm; mobile phase: [water (0.1% ammonia water)-acetonitrile]; gradient: (acetonitrile): 55%-70%), lyophilized to obtain compound 17D. MS m/z=714.6 [M+H]⁺. ¹H NMR (400 MHZ, CD₃OD) δ ppm 6.90 (d, J=8.0 Hz, 1H), 5.14 (dd, J=12.0, 4.0 Hz, 1H), 4.80 (d, J=12.0 Hz, 1H), 4.65 (d, J=12.0 Hz, 1H), 4.37 (d, J=8.0 Hz, 1H), 4.11-4.05 (m, 2H), 3.80-3.76 (m, 1H), 3.74-3.60 (m, 4H), 3.58-3.48 (m, 6H), 3.42-3.39 (m, 2H), 3.21 (dd, J=16.0, 12.0 Hz, 1H), 3.03 (d, J=12 Hz, 1H), 2.87-2.76 (m, 2H), 2.58-2.52 (m, 1H), 2.22-2.11 (m, 1H), 2.01-1.98 (m, 5H), 1.94-1.88 (m, 1H), 1.85-1.80 (m, 4H), 1.79-1.66 (m, 3H), 1.61-1.54 (m, 1H).

### Example 18

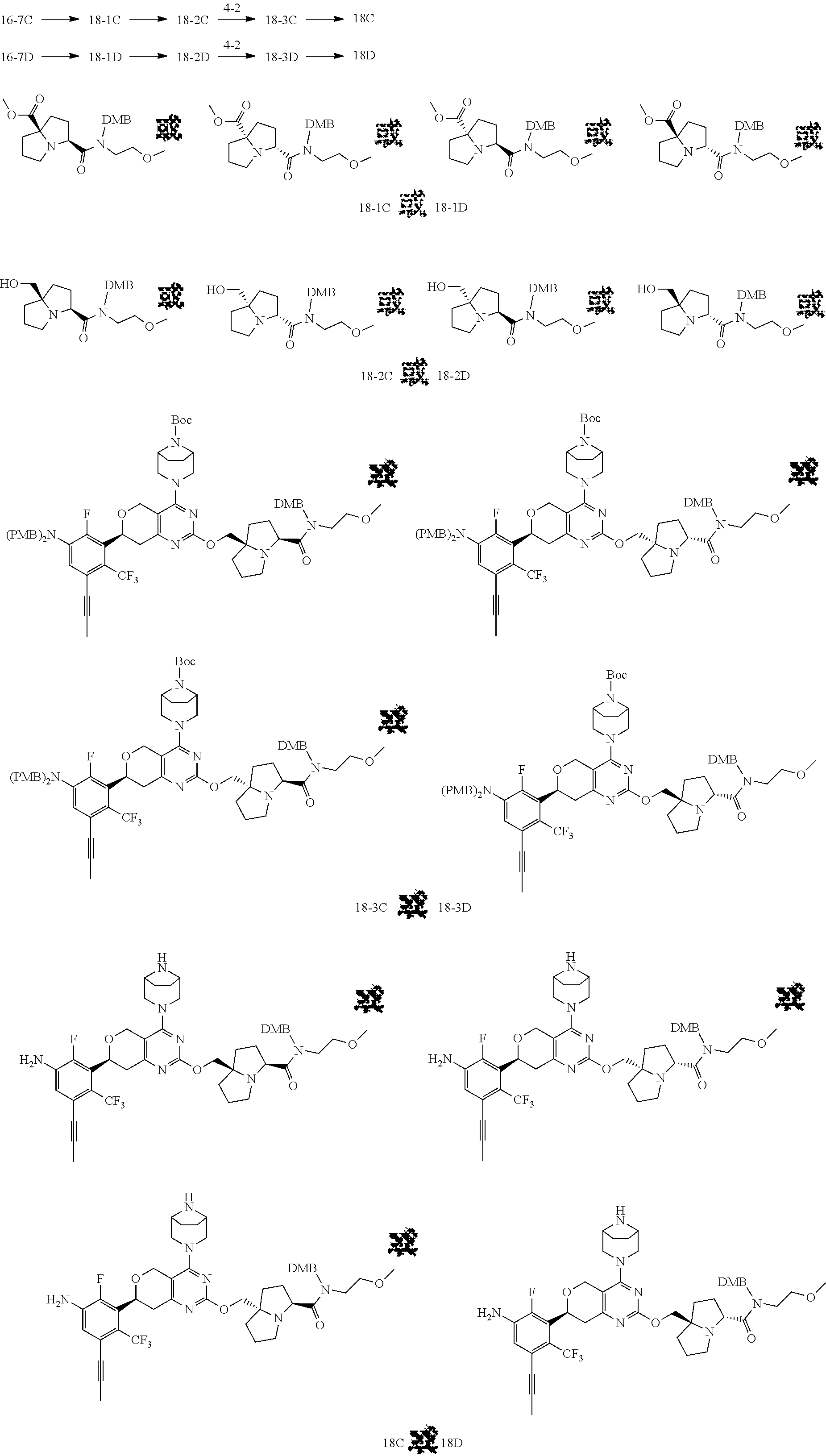

### Step 1

Compound 16-7C (0.15 g, 703.47 μmol) was dissolved in dichloromethane (10 mL), N,N-diisopropylethylamine (3.52 mmol, 612.65 μL), N-(2,4-dimethoxybenzyl)-2-methoxyethanamine (316.96 mg, 1.41 mmol), N,N,N′,N′-tetramethyl-O-7-azabenzotriazole-1-yl hexafluorourea phosphate (534.96 mg, 1.41 mmol), and reacted at 25° C. for 1 hour. The organic phase was concentrated under reduced pressure and column chromatography separated (petroleum ether: ethyl acetate=3:1) to obtain compound 18-1C. MS m/z=421.4 [M+H]⁺.

With reference to step 1, compound 16-7C was replaced with compound 16-7D as the raw material to obtain compound 18-1D. MS m/z=421.4 [M+H]⁺.

### Step 2

Compound 18-1C (0.27 g, 642.10 μmol) was dissolved in tetrahydrofuran (3 mL), sodium borohydride (485.84 mg, 12.84 mmol) and lithium chloride (27.22 mg, 642.10 μmol) were added, reacted at 50° C. for 16 hours. After cooling to room temperature, add water (3 mL) to quench the reaction, extract with ethyl acetate (5 mL*3), combine the organic phases, concentrate under reduced pressure to obtain compound 18-2C. MS m/z=393.3 [M+H]⁺.

With reference to step 2, compound 18-1C was replaced with compound 18-1D as the raw material to obtain compound 18-2D. MS m/z=393.3 [M+H]⁺.

### Step 3

Compound 18-2C (0.18 g. 321.03 μmol) was dissolved in tetrahydrofuran (5 mL) and then cooled to 0° C. Sodium hydrogen (17.12 mg, 428.04 μmol, 60% purity) was added, and the reaction was performed at 0° C. for 0.5 hours. Add 4-2 (188.33 mg, 214.02 μmol), and react at 25° C. for 0.5 hour. Saturated aqueous ammonium chloride solution (15 mL) was quenched, extracted with ethyl acetate (5 mL*3), the organic phase was combined and concentrated under reduced pressure, and column chromatography separated (petroleum ether:ethyl acetate=1:1) to obtain compound 18-3C. MS m/z=1192.8 [M+H]⁺.

With reference to step 3, compound 18-2C was replaced with compound 18-2D as the raw material to obtain compound 18-3D. MS m/z=1192.8 [M+H]⁺.

### Step 4

Compounds 18-3C (0.20 g, 167.74 μmol) were dissolved in trifluoroacetic acid (10 mL) and reacted at 50° C. for 0.5 hours. The organic phase was concentrated under reduced pressure to obtain the crude product, and the crude product was prepared for high performance liquid chromatography separation (chromatographic column: Waters Xbridge, 250*19 mm, 5 mm; mobile phase: [water (0.1% ammonia water)-acetonitrile]; gradient: (acetonitrile): 55%-70%, and lyophilized to obtain compound 18C. MS m/z=702.6 [M+H]⁺. ¹H NMR (400 MHZ, CD₃OD) δ ppm 6.90 (d, J=8.0 Hz, 1H), 5.15 (dd, J=12.0, 4.0 Hz, 1H), 4.78 (s, 1H), 4.65 (d, J=12.0 Hz, 1H), 4.17-4.06 (m, 3H), 3.77 (dd, J=8.0, 4.0 Hz, 1H), 3.53 (d, J=12.0 Hz, 3H), 3.49-3.36 (m, 5H), 3.34 (s, 3H), 3.21 (dd, J=16.0, 12.0 Hz, 1H), 3.04 (d, J=12.0 Hz, 1H), 2.95-2.91 (m, 1H), 2.85 (dd, J=16.0, 4.0 Hz, 1H), 2.78-2.69 (m, 1H), 2.13-2.06 (m, 2H), 2.04-1.96 (m, 5H), 1.94-1.89 (m, 4H), 1.85-1.82 (m, 2H), 1.76-1.66 (m, 2H).

Weigh compound 18-3D (0.15 g, 125.80 μmol), add dichloromethane (2.5 mL), add trifluoroacetic acid (2.5 mL), and react at 25° C. for 0.5 hour. The organic phase was concentrated under reduced pressure, and the crude product was prepared for high performance liquid chromatography separation (chromatographic column: Waters Xbridge, 250*19 mm, 5 mm; mobile phase: [water (0.1% ammonia water)-acetonitrile]; gradient: (acetonitrile): 55%-70%. After lyophilization, compound 18D was obtained. MS m/z=702.6 [M+H]⁺. ¹H NMR (400 MHZ, CD₃OD) δ ppm 6.90 (d, J=8.0 Hz, 1H), 5.15 (dd, J=12.0, 4.0 Hz, 1H), 4.78 (s, 1H), 4.65 (d, J=12.0 Hz, 1H), 4.15 (t, J=12.0 Hz, 2H), 4.05 (d, J=12.0 Hz, 1H), 3.78-3.75 (m, 1H), 3.53 (d, J=8.0 Hz, 3H), 3.49-3.36 (m, 5H), 3.34 (s, 3H), 3.21 (dd, J=16.0, 12.0 Hz, 1H), 3.04 (d, J=12.0 Hz, 1H), 2.95-2.90 (m, 1H), 2.84 (dd, J=16.0, 4.0 Hz, 1H), 2.76-2.70 (m, 1H), 2.15-2.07 (m, 2H), 2.01-1.94 (m, 5H), 1.95-1.87 (m, 4H), 1.86-1.80 (m, 2H), 1.76-1.66 (m, 2H).

### Example 19

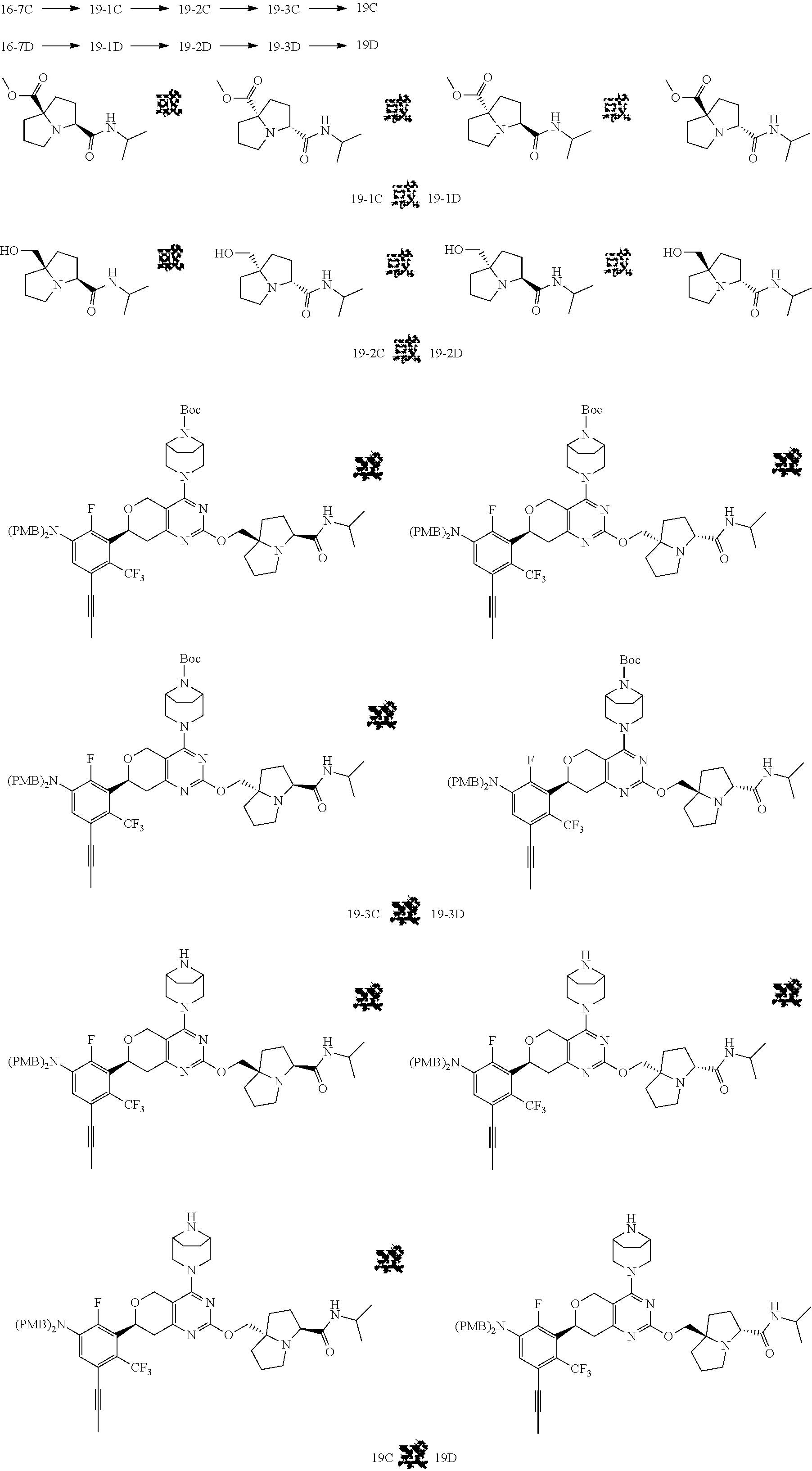

### Step 1

Weigh compound 16-7C (0.40 g, 1.86 mol), add with dichloromethane (6 mL), isopropylamine (166.33 mg, 2.81 mmol), 2-(7-azabenzotriazole)-N,N,N′,N′-tetramethyluronium hexafluorophosphate (1.43 g, 3.75 mmol), and N, N-diisopropylethylamine (0.73 g, 5.63 mol) for reacting at 25° C. for 3 hours. Add water (10 mL), dichloromethane (10 mL*3) for extraction, and concentrate the organic phase under reduced pressure to obtain compound 19-1C, MS m/z=255.0 [M+H]⁺.

With reference to step 1, compound 16-7C was replaced with compound 16-7D as the raw material to obtain compound 19-1D. MS m/z=255.0 [M+H]⁺.

### Step 2

Weigh compound 19-1C (0.10 g, 0.39 mmol), add anhydrous tetrahydrofuran (3 mL) solution under nitrogen protection, cool to 0° C., add tetrahydrofuran solution of lithium tetrahydroaluminum (1.18 mmol, 0.47 ml, 2.5 M), and react at 25° C. for 15 minutes. Add 0.2 mL of water to the reaction solution at 0° C., add 0.2 mL of 15% NaOH solution to quench the reaction, stir for 10 minutes, filter the reaction solution, wash the filter cake with 5 mL of tetrahydrofuran, and concentrate the filtrate to obtain compound 19-2C, MS m/z=227.1 [M+H]⁺.

With reference to step 2, compound 19-1C was replaced with compound 19-1D as the raw material to obtain compound 19-2D MS m/z=227.1 [M+H]⁺.

### Step 3

Weigh compound 19-2C (50.0 mg, 0.22 mmol), add anhydrous tetrahydrofuran (5 mL), add sodium hydride (15.91 mg, 0.66 mmol, 60%) under ice bath at 0° C., stir at 25° C. for 30 minutes, and add compound 4-2 (0.20 g, 0.22 mmol), and react at 25° C. for 1 hour. Add water (5 mL) to quench the reaction solution, extract with ethyl acetate (5 mL*3), concentrate the organic phase under reduced pressure, and separate by column chromatography (dichloromethane:methanol=20:1) to obtain compound 19-3C, MS m/z=1026.8 [M+H]⁺.

With reference to step 3, compound 19-2C was replaced with compound 19-2D as the raw material to obtain compound 19-3D MS m/z=1026.8 [M+H]⁺.

### Step 4

Compound 19-3C (50 mg, 0.05 mmol) was dissolved in dichloromethane (2 mL), to which trifluoroacetic acid (1 mL) was added, and the reaction solution was reacted at 25° C. for 0.5 hour. The reaction solution was concentrated under reduced pressure to obtain crude product. The crude product was prepared for high performance liquid chromatography separation (chromatographic column: Waters SunFire, 250*19 mm, 5 μm; mobile phase: [water (0.05% formic acid)-acetonitrile]; acetonitrile: 45%-75%. After lyophilization, the formate of compound 19C was obtained. MS m/z=686.5 [M+H]⁺. 1H NMR (400 MHZ, CD₃OD) δ ppm 8.45 (s, 1.52H), 6.91 (d, J=8.4 Hz, 1H), 5.20-5.16 (m, 1H), 4.84-4.79 (m, 1H), 4.70 (d, J=13.7 Hz, 1H), 4.33 (d, J=11.2 Hz, 1H), 4.26-4.10 (m, 2H), 4.08-3.97 (m, 4H), 3.76-3.73 (m, 1H), 3.58 (d, J=13.4 Hz, 1H), 3.27-3.21 (m, 3H), 3.03-3.01 (m, 1H), 2.93-2.88 (m, 1H), 2.26-2.06 (m, 8H), 2.01-1.94 (m, 5H), 1.92-1.87 (m, 2H), 1.17 (dd, J=6.6, 1.5 Hz, 6H).

Compound 19-3D (50 mg, 0.05 mmol) was dissolved in dichloromethane (2 mL), to which trifluoroacetic acid (1 mL) was added, and the reaction solution was reacted at 25° C. for 0.5 hour. The reaction solution was concentrated under reduced pressure to obtain crude product. The crude product was prepared for high performance liquid chromatography separation (chromatographic column: Waters SunFire, 250*19 mm, 5 μm; mobile phase: [water (0.05% formic acid)-acetonitrile]; acetonitrile: 45%-75%. After lyophilization, compound 19D formate was obtained. MS m/z=686.5 [M+H]⁺. ¹H NMR (400 MHZ, CD₃OD) δ ppm 8.36 (s, 1.59H), 6.81 (d, J=8.4 Hz, 1H), 5.10-5.07 (m, 1H), 4.81-4.78 (m, 1H), 4.61 (d, J=13.6 Hz, 1H), 4.22-4.08 (m, 3H), 3.94-3.88 (m, 4H), 3.63 (d, J=13.6 Hz, 1H), 3.48 (d, J-13.2 Hz, 1H), 3.17-3.03 (m, 3H), 2.91-2.78 (m, 2H), 2.15-1.99 (m, 6H), 1.98-1.92 (m, 6H), 1.84-1.76 (m, 3H), 1.07 (d, J=6.6 Hz, 6H).

### Example 20

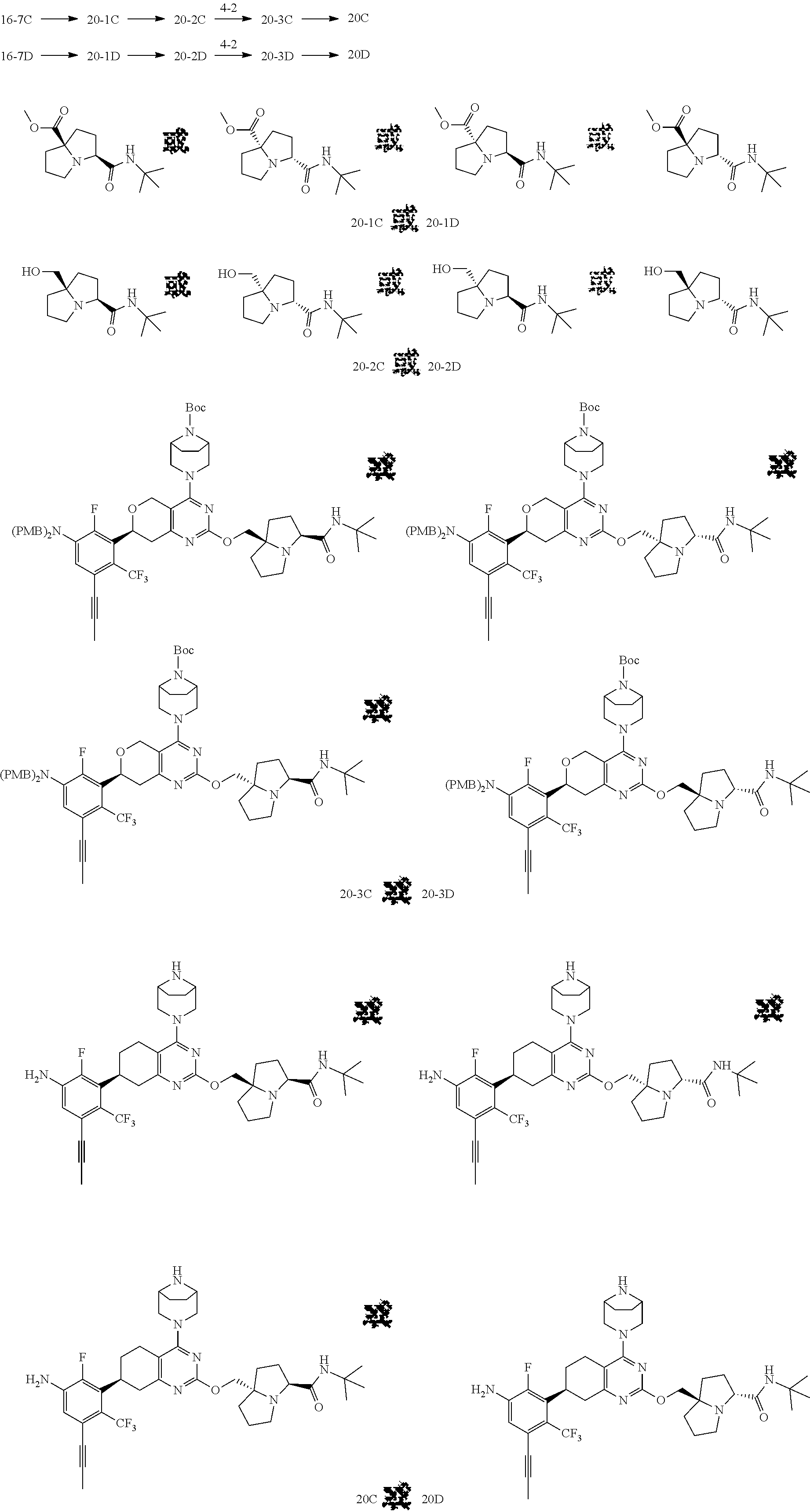

### Step 1

Compound 16-7C (100 mg, 468.98 μmol) was dissolved in N,N-dimethylformamide (2 mL), 2-chloro-1-methylpyridine iodide (179.72 mg, 703.47 μmol), triethylamine (142.37 mg, 1.41 mmol), tert-butylamine (51.45 mg, 703.47 μmol) was added and reacted at 25° C. for 2 hours. Add water (10 mL), extract with ethyl acetate (10 mL*3), combine the extracted organic phases, wash with saturated brine (10 mL), dry with anhydrous sodium sulfate, filter, and concentrate under reduced pressure. Column chromatography separation (petroleum ether:ethyl acetate=1:1) yielded compound 20-1C. MS m/z=269.0 [M+H]⁺.

With reference to step 1, compound 16-7C was replaced with compound 16-7D as the raw material to obtain compound 20-1D. MS m/z=269.0 [M+H]⁺.

### Step 2

Compound 20-1C (300 mg, 1.12 mmol) was dissolved in anhydrous tetrahydrofuran (5 mL), cooled to 0° C. under nitrogen protection, and lithium borohydride in tetrahydrofuran solution (1.12 mL, 2 M) was added dropwise, and heated to 50° C. for stirring and reacting for 8 hours. Add 3 mL of water to quench the reaction, filter, and concentrate the filtrate under reduced pressure to obtain the crude product. The crude product was separated by column chromatography (dichloromethane:methanol=10:1) to obtain compound 20-2C. MS m/z=241.1 [M+H]⁺.

With reference to step 2, compound 20-1C was replaced with compound 20-1D as the raw material to obtain compound 20-2D. MS m/z=241.1 [M+H]⁺.

### Step 3

Compound 20-2C (81.90 mg, 340.93 μmol) was dissolved in anhydrous tetrahydrofuran (5 mL), cooled to 0° C., and sodium hydride (20.50 mg, 853.75 μmol, 60% purity) was slowly added therein, and then heated to 25° C. for stirring and reaction for 0.5 hours. Compound 4-2 (200 mg, 227.28 μmol) was added and reacted at 25° C. for 2 hours Add 3 mL of water to quench the reaction, extract with ethyl acetate ((5 mL*3), combine the extracted organic phases, wash with saturated brine (10 mL), dry with anhydrous sodium sulfate, filter, and concentrate under reduced pressure. Column chromatography (petroleum ether:ethyl acetate=1:1) yielded compound 20-3C. MS m/z=1040.8 [M+H]⁺.

With reference to step 3, compound 20-2C was replaced with compound 20-2D as the raw material to obtain compound 20-3D. MS m/z=1040.8 [M+H]⁺.

### Step 4

Compounds 20-3C (100 mg, 96.14 μmol) were dissolved in anhydrous methylene chloride (3 mL), to which trifluoroacetic acid (1 mL) was added, and the reaction solution was reacted at 25° C. for 2 hours. The reaction solution was concentrated under reduced pressure to obtain crude product. The crude product was prepared for high performance liquid chromatography (column: Xtimate C18 150*40 mm*5 μm; mobile phase: [water (0.05% formic acid)-acetonitrile]; (acetonitrile): 10%-40%); after lyophilization, the formate of compound 20C was obtained. 20C MS m/z=700.6 [M+H]⁺, ¹H NMR (400 MHZ, CD₃OD) δ ppm 8.51 (s, 1H), 6.91 (d, J=8.4 Hz, 1H), 5.20-5.15 (m, 1H), 4.82 (d, J=13.6 Hz, 1H), 4.69 (d, J=13.6 Hz, 1H), 4.27-4.14 (m, 3H), 3.88 (s, 3H), 3.68 (d,/=13.2 Hz, 1H), 3.54 (d, J=13.2 Hz, 1H), 3.28-3.15 (m, 2H), 3.10-3.04 (m, 1H), 2.95-2.86 (m, 2H), 2.21-2.12 (m, 3H), 2.02-1.76 (m, 12H), 1.36 (s, 9H).

Compound 20-3D (150 mg, 144.21 μmol) was dissolved in methylene chloride (4.5 mL), and then trifluoroacetic acid (1.5 mL) was added to the reaction solution, which was reacted at 25° C. for 2 hours. The reaction solution was concentrated under reduced pressure to obtain crude product. The crude product was prepared by high performance liquid chromatography separation (column: Xtimate C18 150*40 mm*5 μm; mobile phase: [water (0.1% ammonium bicarbonate)-acetonitrile]; 35%-53%. After lyophilization, compound 20D was obtained. 20D MS m/z=700.6 [M+H]′, ¹H NMR (400 MHZ, CD₃OD) δ ppm 6.90 (d, J=8.4 Hz, 1H), 5.17-5.12 (m, 1H), 4.80 (d, J=13.6 Hz, 1H), 4.65 (d, J=13.6 Hz, 1H), 4.18-4.12 (m, 2H), 4.05 (d, J=16.0 Hz, 1H), 3.71-3.68 (m, 1H), 3.54-3.51 (m, 3H), 3.43-3.40 (m, 1H), 3.25-3.18 (m, 1H), 3.04 (d, J=16.0 Hz, 1H), 2.93-2.71 (m, 3H), 2.14-2.09 (m, 2H), 2.06-2.01 (m, 4H), 1.92-1.83 (m, 7H), 1.74-1.96 (m, 2H), 1.35 (s, 9H).

### Example 21

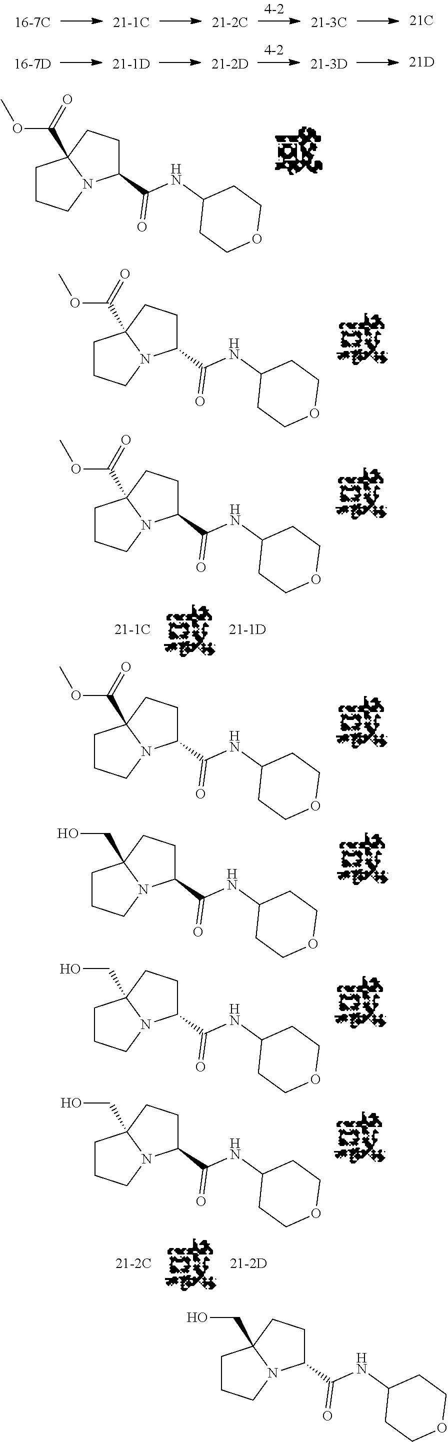

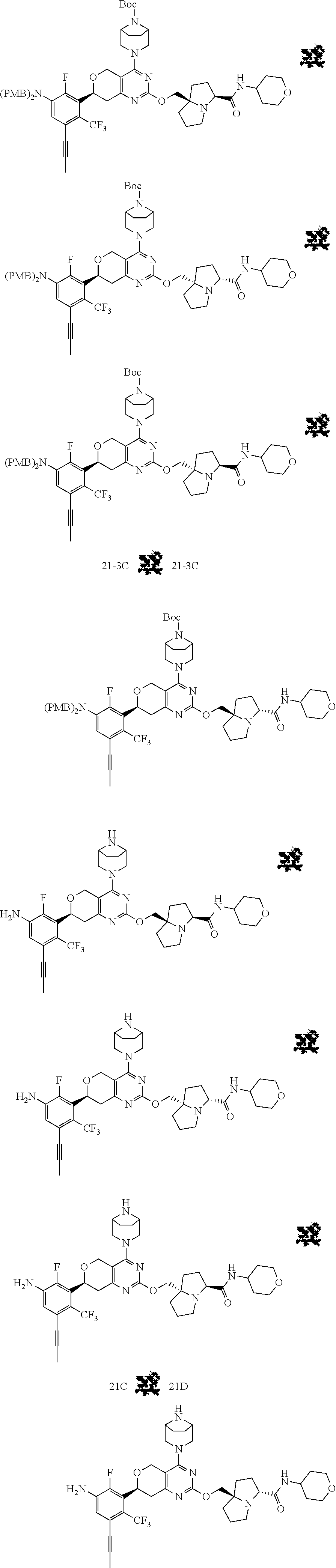

### Step 1

Compound 16-7C (150 mg, 703.47 μmol) was dissolved in N,N-dimethylformamide (2 mL), 4-aminotetrahydrofuran (106.73 mg, 1.06 mmol), N-methylimidazole (173.27 mg, 2.11 μmol), tetramethyl chlorourea hexafluorophosphate (296.07 mg, 1.06 mmol) was added and reacted at 25° C. for 3 hours. Add water (10 mL), extract with ethyl acetate (10 mL*3), combine the extracted organic phases, wash with saturated brine (5 mL), dry with anhydrous sodium sulfate, filter, and concentrate under reduced pressure. Column chromatography separation (dichloromethane:methanol=20:1) yielded compound 21-1C. MS m/z=297.1 [M+H]⁺.

With reference to step 1, compound 16-7C was replaced with compound 16-7D as the raw material to obtain compound 21-1D. MS m/z=297.1 [M+H]⁺.

### Step 2

Compound 21-1C (300 mg, 1.01 mmol) was dissolved in anhydrous methanol (5 mL), sodium methanol (21.90 mg, 404.91 μmol) and sodium borohydride (191.50 mg, 5.06 mmol) were added, and reacted at 50° C. for 24 hours. Add 3 mL saturated ammonium chloride aqueous solution to quench reaction, filter, and filtrate concentration under reduced pressure to obtain crude product Column chromatography separation (dichloromethane:methanol=10:1) yielded compound 21-2C. MS m/z=269.2 268.5 [M+H]⁺.

With reference to step 2, compound 21-1C was replaced with compound 21-1D as the raw material to obtain compound 21-2D. MS m/z=269.2 [M+H]⁺.

### Step 3

Compound 21-2C (100 mg, 372.65 μmol) was dissolved in anhydrous tetrahydrofuran (5 mL), cooled to 0° C., and sodium hydride (44.70 mg, 1.12 mmol, 60% purity) was slowly added to it, and the temperature was increased to 25° C. for stirring and reaction for 0.5 hours. Compound 4-2 (163.90 mg, 186.32 μmol) was added and reacted at 25° C. for 2 hours. Add 3 mL of water to quench the reaction, extract with ethyl acetate (5 mL*3), combine the extracted organic phases, wash with saturated brine (10 mL), dry with anhydrous sodium sulfate, filter, and concentrate under reduced pressure. Column chromatography (dichloromethane:methanol=20:1) yielded compound 21-3C. MS m/z=1068.5 [M+H]⁺.

With reference to step 3, compound 21-2C was replaced with compound 21-2D as the raw material to obtain compound 21-3D. MS m/z=1068.5 [M+H]⁺.

### Step 4

Compounds 21-3C (114 mg, 106.72 μmol) were dissolved in anhydrous dichloromethane (0.9 mL), to which trifluoroacetic acid (0.3 mL) was added and reacted at 25° C. for 1 hour. The reaction solution was concentrated under reduced pressure to obtain crude product. The crude product was prepared by high performance liquid chromatography separation (column: Xtimate C18 150*40 mm*5 μm; mobile phase: [water (0.05% formic acid)-acetonitrile]; acetonitrile]: 10%-60%). After lyophilization, the formate of compound 21C is obtained. 21C MS m/z=728.5 [M+H]⁺, ¹H NMR (400 MHz, DMSO-d₆) δ ppm 8.22 (s, 1.38H), 7.97 (d, J=7.7 Hz, 1H), 6.88 (d, J=8.4 Hz, 1H), 6.02 (s, 2H), 5.05-5.01 (m, 1H), 4.75 (d, J=13.7 Hz, 1H), 4.57 (d, J=13.7 Hz, 1H), 3.94-3.90 (m, 3H), 3.83-3.79 (m, 3H), 3.64 (s, 2H), 3.54-3.51 (m, 1H), 3.44-3.41 (m, 1H), 3.35-3.30 (m, 4H), 3.09-3.02 (m, 1H), 2.98 (d, J=12.5 Hz, 1H), 2.82-2.77 (m, 1H), 2.75-2.70 (m, 1H), 2.66-2.59 (m, 1H), 2.03 (s, 3H), 1.97-1.80 (m, 6H), 1.80-1.52 (m, 8H), 1.47-1.37 (m, 2H).

Compounds 21-3D (200 mg, 187.23 μmol) were dissolved in anhydrous dichloromethane (5 mL), to which trifluoroacetic acid (5 mL) was added and reacted at 25° C. for 1 hour. The reaction solution was concentrated under reduced pressure to obtain crude product. The crude product was prepared by high performance liquid chromatography (column: Xtimate C18 150*40 mm*5 μm; mobile phase: [water (0.05% formic acid)-acetonitrile]; acetonitrile]; 10%-60%). After lyophilization, the formate of compound 21D was obtained. 21D MS m/z=728.5 [M+H]⁺, ¹H NMR (400 MHZ, DMSO-d₆) δ ppm 8.14 (s, 0.56H), 7.96 (d, J=7.8 Hz, 1H), 6.88 (d, J=8.3 Hz, 1H), 6.01 (s, 2H), 5.04-5.00 (m, 1H), 4.74 (d, J=13.7 Hz, 1H), 4.54 (d, J=13.7 Hz, 1H), 4.05-3.92 (m, 4H), 3.85-3.79 (m, 4H), 3.64-3.51 (m, 2H), 3.46 (d, J=13.3 Hz, 1H), 3.40-3.35 (m, 3H), 3.14-3.06 (m, 2H), 2.87-2.68 (m, 2H), 2.67-2.52 (m, 1H), 2.03 (s, 3H), 1.91-1.53 (m, 14H), 1.46-1.38 (m, 2H).

### Example 22

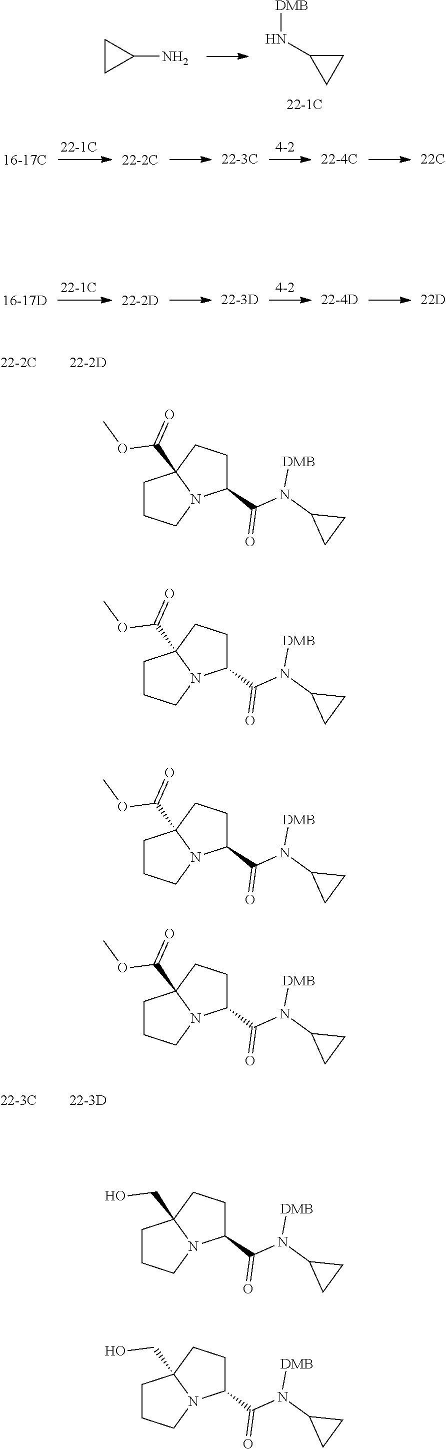

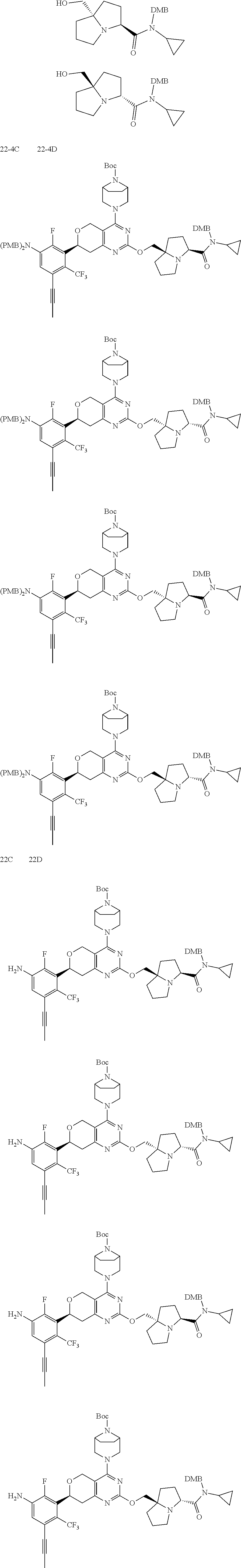

### Step 1

Add 2,4-dimethoxybenzaldehyde (500 mg, 3.01 mmol) to anhydrous tetrahydrofuran (5 mL), add cyclopropylamine (6.02 mmol, 416.97 μL) and glacial acetic acid (18.00 mg, 300.89 μmol), and react at 25° C. for 2 hours. Sodium borohydride (567.20 mg, 9.03 mmol) was added to the above system and stirred and reacted at 25° C. for 16 hours. Water (5 mL) was added for quenching, dichloromethane (10 mL*3) was extracted, the extracted organic phase was combined, washed with saturated brine (10 mL), dried over anhydrous sodium sulfate, filtered, concentrated under reduced pressure to obtain compound 22-1C. MS m/z=208.2 [M+H]⁺.

### Step 2

Compound 16-7C (140 mg, 656.56 μmol) was dissolved in anhydrous dichloromethane (6 mL), N,N-diisopropylethylamine (3.28 mmol, 571.80 μL) was added, compound 22-1C (176.90 mg, 853.54 mmol), N,N,N′,N′-tetramethyl-O)-(7-azabenzotriazole-1-yl) hexafluorourea phosphate (499.31 mg, 1313.14 μmol) was added, reacted at 25° C. for 16 hours. Water (5 mL) was added for quenching, extracted with ethyl acetate (10 mL*3), the extracted organic phase was combined, washed with saturated brine (10 mL), dried with anhydrous sodium sulfate, filtered, concentrated under reduced pressure, and column chromatography separation was performed (petroleum ether:ethyl acetate=3:1) to obtain compound 22-2C. MS m/z=403.2 [M+H]⁺.

With reference to step 2, compound 16-7C was replaced with compound 16-7D as the raw material to obtain compound 22-2D. MS m/z=403.2 [M+H]⁺.

### Step 3

Compound 22-2C (170 mg, 422.38 μmol) was dissolved in anhydrous tetrahydrofuran (3 mL) and anhydrous methanol (3 mL), sodium borohydride (79.90 mg, 2.11 mmol) was added, lithium chloride (17.90 mg, 422.38 μmol) was reacted at 50° C. for 2 hours. Add water (0.5 mL) to quench, filter, and concentrate the filtrate under reduced pressure to obtain compound 22-3C. MS m/z=375.2 [M+H]⁺. With reference to step 3, compound 22-2C was replaced with compound 22-2D as the raw material to obtain compound 22-3D. MS m/z=375.2 [M+H]⁺

### Step 4

Compound 22-3C (104 mg, 277.72 μmol) was dissolved in anhydrous tetrahydrofuran (5 mL), sodium hydride (33.30 mg, 833.17 μmol, 60% purity) was added, and the reaction was performed at 0° C. for 0.5 hours. Compound 4-2 (150.00 mg, 170.46 μmol) was added and reacted at 25° C. for 1 hour. Add saturated aqueous ammonium chloride solution (10 mL) for quenching, extract with ethyl acetate (10 mL*3), combine the extracted organic phases, wash with saturated brine (10 mL), dry with anhydrous sodium sulfate, filter, concentrate under reduced pressure, and separate by column chromatography (petroleum ether: Ethyl acetate=1:1) to obtain compound 22-4C. MS m/z=1174.5 [M+H]⁺.

With reference to step 4, compound 22-3C was replaced with compound 22-3D as the raw material to obtain compound 22-4D. MS m/z=1174.5 [M+H]⁺.

### Step 5

Compounds 22-4C (150.00 mg, 127.73 μmol) were dissolved in trifluoroacetic acid (5 mL) and reacted at 50° C. for 0.5 hour. The reaction solution was concentrated under reduced pressure to obtain the crude product. The crude product was prepared for high performance liquid chromatography separation (column: Xtimate C18 150*40 mm*5 μm; mobile phase: [water (0.1% ammonia water)-acetonitrile]; gradient: (acetonitrile): 35%-60%, and lyophilized to obtain compound 22C. MS m/z=684.6 [M+H]⁺. ¹H NMR (400 MHZ, CD₃OD) δ ppm 6.90 (d, J=8.4 Hz, 1H), 5.14 (dd, J=11.6 Hz, 4.8 Hz, 1H), 4.79 (d, J=13.6 Hz, 1H), 4.64 (d, J=13.6 Hz, 1H), 4.14-4.04 (m, 3H), 3.68 (dd, J=8.0 Hz, 5.2 Hz, 1H), 3.52-3.49 (m, 3H), 3.42-3.38 (m, 1H), 3.21 (dd, J=17.6 Hz, 11.2 Hz, 1H), 3.03 (d, J=12.4 Hz, 1H), 2.88-2.84 (m, 2H), 2.73-2.65 (m, 2H), 2.11-2.05 (m, 2H), 2.01-1.97 (m, 5H), 1.94-1.81 (m, 6H), 1.76-1.64 (m, 2H), 0.75-0.71 (m, 2H), 0.53-0.48 (m, 2H).

Compounds 22-4D (100.00 mg, 85.16 μmol) were dissolved in trifluoroacetic acid (3 mL) and reacted at 50° C. for 0.5 hour. The reaction solution was concentrated under reduced pressure to obtain the crude product. The crude product was prepared for high performance liquid chromatography separation (chromatographic column: Xtimate C18 150*40 mm*5 μm; mobile phase: [water (0.1% ammonia water)-acetonitrile]; gradient: (acetonitrile: 35%-70%, and lyophilized to obtain compound 22D. MS m/z=684.5 [M+H]⁺. ¹H NMR (400 MHz, CD₃OD) δ ppm 6.90 (d, J=8.4 Hz, 1H), 5.14 (dd, J=11.6, 4.8 Hz, 1H), 4.79 (d, J=13.6 Hz, 1H), 4.64 (d, J=13.6 Hz, 1H), 4.16-4.12 (m, 2H), 4.03 (d, J=10.4 Hz, 1H), 3.68 (dd, J=8.0 Hz, 5.2 Hz, 1H), 3.53-3.50 (m, 3H), 3.42-3.39 (m, 1H), 3.21 (dd, J=17.6 Hz, 11.2 Hz, 1H), 3.03 (d, J=12.4 Hz, 1H), 2.88-2.81 (m, 2H), 2.78-2.66 (m, 2H), 2.12-2.06 (m, 2H), 2.01-1.97 (m, 5H), 1.95-1.81 (m, 6H), 1.77-1.66 (m, 2H), 0.76-0.72 (m, 2H), 0.52-0.48 (m, 2H).

### Example 23

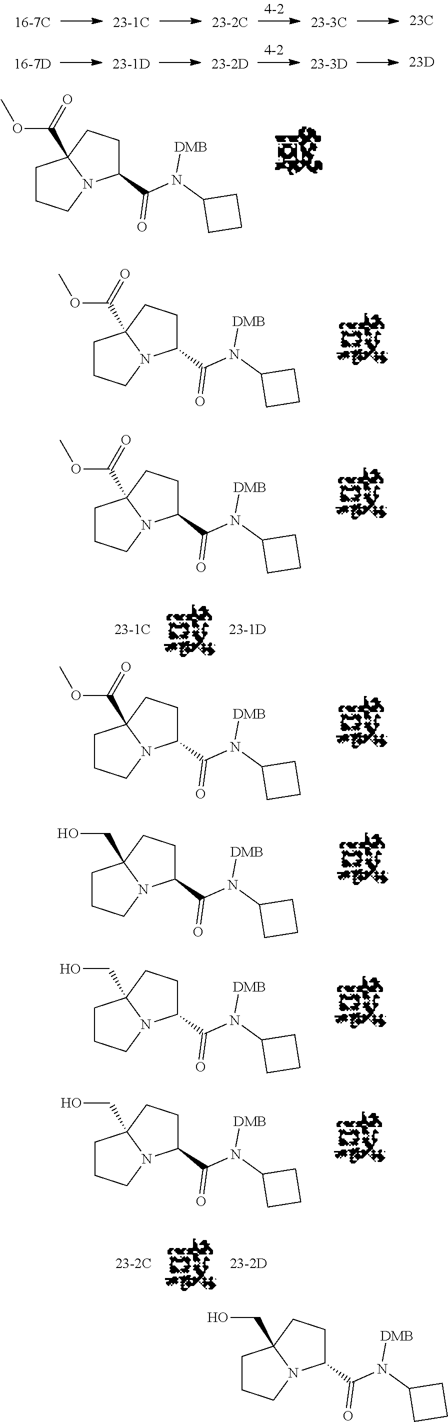

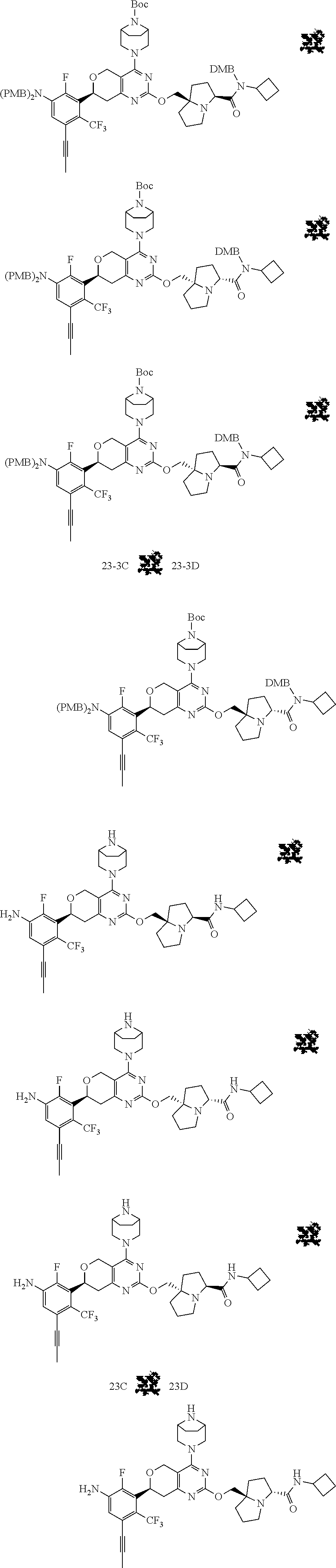

### Step 1

Compound 16-7C (0.20 g, 937.96 μmol) was dissolved in dimethylformamide (2 mL), followed by addition of N,N-diisopropylethylamine (606.12 mg, 4.69 mmol), N,N,N′,N′-tetramethyl-O-(7-azabenzotriazole-1-yl) hexafluorourea phosphate (713.28 mg, 1.88 mmol), N-[(2,4-dimethoxyphenyl)methyl]cyclobutamine 207.56 mg, 937.96 μmol), and reacted at 25° C. for 12 hours. The reaction solution was added to water (10 mL), extracted with ethyl acetate (20 mL*3), concentrated under reduced pressure, and column chromatography separated (petroleum ether:ethyl acetate=5:1) to obtain compound 23-1C. MS m/z=417.4 [M+H]⁺.

With reference to step 1, compound 16-7C was replaced with compound 16-7D as the raw material to obtain compound 23-1D. MS m/z=417.4 [M+H]⁺.

### Step 2

Compound 23-1C (0.20 g, 480.18 μmol) was dissolved in methanol (2 mL), sodium methoxide (2.59 mg, 48.02 μmol) and boron-hydrogenated sodium (90.83 mg, 2.40 mmol) were added at 0° C., and reacted at 25° C. for 16 hours. The reaction was quenched by adding saturated ammonium chloride (10 mL), extracted with ethyl acetate (20 mL), dried over anhydrous sodium sulfate, filtered, and concentrated under reduced pressure to obtain compound 23-2C. MS m/z=389.3 [M+H]⁺.

With reference to step 2, compound 23-1C was replaced with compound 23-1D as the raw material to obtain compound 23-2D. MS m/z=389.3 [M+H]⁺.

### Step 3

Compound 23-2C (50.00 mg, 128.70 μmol) was dissolved in tetrahydrofuran (1 mL), sodium hydrogen (51.48 mg, 1.29 mmol, 60% purity) was added at 0° C., stirred for 0.5 hours, and compound 4-2 (113.25 mg, 128.70 μmol) was finally added, and reacted at 25° C. for 2 hours. The reaction was quenched by adding water (10 mL), extracted with ethyl acetate (20 mL), concentrated under reduced pressure, and separated by column chromatography (petroleum ether:ethyl acetate=0:1) to obtain compound 23-3C. MS m/z=1188.7 [M+H]⁺.

With reference to step 3, compound 23-2C was replaced with compound 23-2D as the raw material to obtain compound 23-3D. MS m/z=1188.7 [M+H]⁺.

### Step 4

Compounds 23-3C (17 mg, 14.31 μmol) were dissolved in methylene chloride (1 mL), trifluoroacetic acid (0.3 mL) was added, and reacted at 25° C. for 0.5 hour. The reaction solution was concentrated under reduced pressure to obtain crude product. Crude product was prepared for high performance liquid chromatography separation (chromatographic column: Waters Xbridge, 250*19 mm, 5 mm; mobile phase: [water (0.1% ammonia)-acetonitrile]; gradient: (acetonitrile): 55%-70%, lyophilized to obtain compound 23C. MS m/z=698.5 [M+H]⁺. ¹H NMR (400 MHZ, CD₃OD) δ ppm 6.90 (d, J=8.0 Hz, 1H), 5.15 (dd, J=12.0, 4.0 Hz, 1H), 4.84-4.79 (m, 1H), 4.62 (d, J=16.0 Hz, 1H), 4.34-4.26 (m, 1H), 4.16-4.06 (m, 3H), 3.74-3.70 (m, 1H), 3.51-3.48 (m, 3H), 3.44-3.41 (m, 1H), 3.25-3.13 (m, 1H), 3.04 (d, J=12.0 Hz, 1H), 2.89-2.82 (m, 2H), 2.76-2.70 (m, 1H), 2.32-2.26 (m, 2H), 2.12-2.06 (m, 2H), 2.04-1.89 (m, 11H), 1.85-1.83 (m, 2H), 1.77-1.71 (m, 4H).

Compound 23-3D (40 mg, 33.66 μmol) was dissolved in methylene chloride (1 mL), trifluoroacetic acid (1 mL) was added, and the solution was stirred and reacted at 25° C. for 0.5 hour. The reaction solution was concentrated under reduced pressure to obtain crude product. The crude product was prepared for high performance liquid chromatography separation (Waters Xbridge, 250*19 mm, 5 mm; mobile phase: [water (0.1% ammonia)-acetonitrile]; gradient: (acetonitrile): 55%-70%). After lyophilization, compound 23D was obtained. MS m/z=698.5 [M+H]⁺. ¹H NMR (400 MHZ, CD₃OD) δ ppm 6.90 (d, J=8.0 Hz, 1H), 5.15 (dd, J=12.0, 4.0 Hz, 1H), 4.83-4.78 (m, 1H), 4.65 (d, J=16.0 Hz, 1H), 4.34-4.26 (m, 1H), 4.18-4.12 (m, 2H), 4.04 (d, J=8.0 Hz, 1H), 3.73-3.69 (m, 1H), 3.53 (d, J=12.0 Hz, 3H), 3.43-3.41 (d, J=8.0 Hz, 1H), 3.25-3.18 (m, 1H), 3.04 (d, J=12.0 Hz, 1H), 2.91-2.82 (m, 2H), 2.76-2.70 (m, 1H), 2.33-2.26 (m, 2H), 2.11-2.07 (m, 2H), 2.01-1.90 (m, 11H), 1.85-1.83 (m, 2H), 1.78-1.68 (m, 4H).

### Example 24

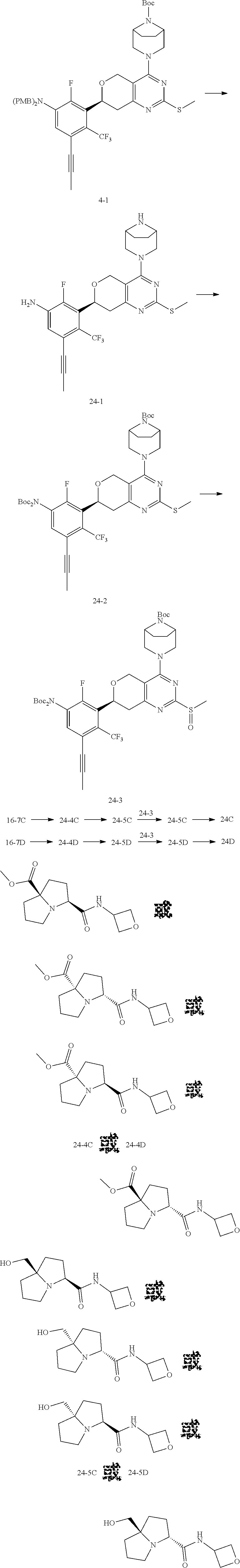

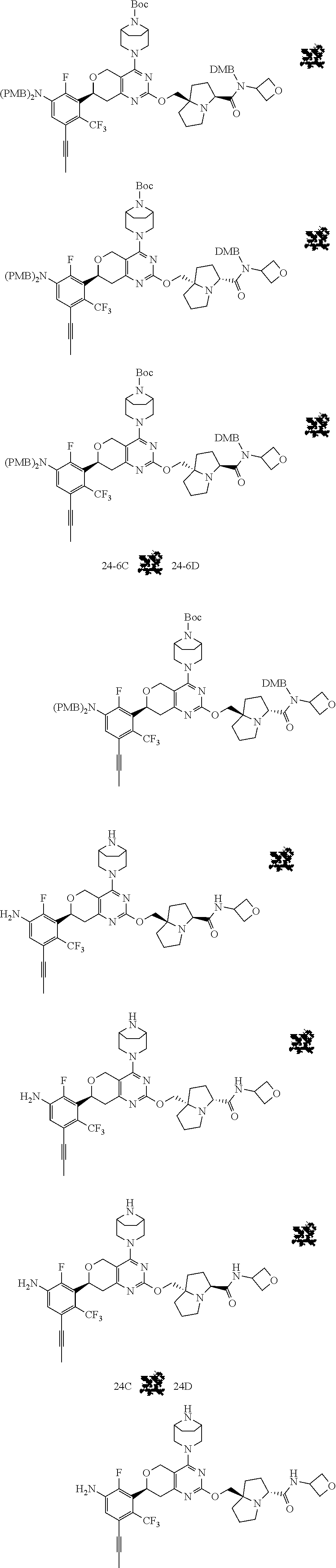

### Step 1

Compound 4-1 (280 mg, 0.33 mmol) was dissolved in dichloromethane (3 mL), and then trifluoroacetic acid (376 mg, 3.30 mmol) was added. The resulting reaction solution was stirred for 3 hours at 25° C. under nitrogen protection. The reaction solution was concentrated under reduced pressure, ethyl acetate 3 mL was added to the residue, and then hydrogen chloride/ethyl acetate solution (4M, 1 mL) was added to it, and the reaction was stirred at 25° C. for 1 hour, and concentrated under reduced pressure to obtain the hydrochloride salt of compound 24-1.

### Step 2

The hydrochloride salt of compound 24-1 (160 mg), triethylamine (159.50 mg, 1.58 mol) was added to dichloromethane (5 mL), then di-tert-butyl dicarbonate ester (1.38 g, 6.30 mmol) and 4-dimethylaminopyridine (38.51 mg, 0.33 mmol) were added, and the reaction solution was stirred for 12 hours under nitrogen protection at 25° C. The reaction solution was concentrated under reduced pressure to obtain crude product. The crude product was purified by column chromatography (petroleum ether:ethyl acetate=5:1) to obtain compound 24-2. MS m/z=808.6 [M+H]⁺.

### Step 3

Compound 24-2 (240 mg, 0.30 mmol) was dissolved in tetrahydrofuran (5 mL), and then metachloroperoxybenzoic acid (61.52 mg, 0.35 mmol, 85% purity) was added. The obtained reaction solution was stirred and reacted at 25° C. for 0.5 hours under nitrogen protection. The reaction solution was concentrated under reduced pressure to obtain compound 24-3. MS m/z=824.5 [M+H]⁺.

### Step 4

Compound 16-7C (180 mg, 0.84 mmol) was dissolved in N,N-dimethylformamide (4 mL), benzotriazazole-N,N′,N′,N′-tetramethyl-uronium-hexafluorophosphate (641.95 mg, 1.69 mmol) and N,N-diisopropylethylamine (218.42 mg, 1.69 mmol) were added, and the reaction mixture was stirred at 25° C. for 30 minutes. In the above mixture, it was added oxetan-3-amine hydrochloride (92.09 mg, 1.26 mmol) and the resulting mixture was stirred at 25° C. for 16 hours. The reaction mixture was diluted with water (20 mL) and extracted with ethyl acetate (50 mL), the organic phase was washed with saturated brine, dried over anhydrous sodium sulfate, filtered, and concentrated under reduced pressure. Column chromatography separation (dichloromethane:methanol=20:1) yielded compound 24-4C. MS m/z=269.18 [M+H]⁺.

With reference to step 4, compound 16-7C was replaced with compound 16-7D as the raw material to obtain compound 24-4D MS m/z=269.18 [M+H]⁺.

### Step 5

Compounds 24-4C (100 mg, 0.37 mmol) were dissolved in tetrahydrofuran (5 mL), sodium borohydride (69.98 mg, 1.85 mmol) and lithium chloride (78.42 mg, 1.85 mmol) were added, and reacted at 25° C. for 12 hours under nitrogen protection. The reaction mixture was diluted with water (10 mL) and extracted with ethyl acetate (30 mL), the organic phase was washed with saturated brine, dried over anhydrous sodium sulfate, filtered and concentrated under reduced pressure. Column chromatography separation (dichloromethane:methanol=10:1) yielded compound 24-5C. MS m/z=241.22 [M+H]⁺.

With reference to step 5, compound 24-4D was replaced with compound 24-4C as the raw material to obtain compound 24-5D. MS m/z=241.22 [M+H]⁺.

### Step 6

Compound 24-5C (70 mg, 0.29 mmol) was dissolved in tetrahydrofuran (3 mL), followed by sodium hydrogen (12 mg. 0.29 mmol, 60% purity) under nitrogen protection, and the reaction was stirred at 25° C. for 0.5 hours, and then a solution of compound 24-3 (210 mg, 0.29 mmol) in tetrahydrofuran (1 mL) was added to the solution, and the reaction was stirred at 25° C. for 2 hours. The reaction solution was dissolved in 10 mL of ethyl acetate, then washed with saturated brine (5 mL), the organic phase was dried, then filtered and the filtrate was concentrated under reduced pressure. Column chromatography separation (dichloromethane:methanol=20:1) yielded compound 24-6C. MS m/z=900.6 [M−100+1]⁺.

With reference to step 6, compound 24-5C was replaced with compound 24-5D as the raw material to obtain compound 24-6D. MS m/z=900.6 [M−100+1]⁺.

### Step 7

Compound 24-6C (10 mg, 10.00 μmol) was dissolved in methylene chloride (3 mL), and then zinc bromide (5 mg, 20.00 μmol) was added. Under nitrogen protection, the reaction was stirred at 25° C. for 2 hours. The reaction solution was concentrated under reduced pressure to obtain crude product.

The crude product was prepared for high performance liquid chromatography separation (chromatographic column: Waters SunFire, 250*19 mm, 5 μm; mobile phase: [water (0.1% formic acid)-acetonitrile]; acetonitrile]: 10%-40%. After lyophilization, the formate of compound 24C was obtained. MS m/z=700.4 [M+H]⁺, ¹H NMR (400 MHZ, CD₃OD) δ ppm 8.39 (s, 2H), 6.92 (d, J=8.4 Hz, 1H), 5.19 (dd, J=11.2, 4.4 Hz, 1H), 4.94 (q, J=6.4 Hz, 1H), 4.88 (d, J=6.8 Hz, 3H), 4.72 (d, J=13.8 Hz, 1H), 4.59 (q, J=5.8 Hz, 2H), 4.41 (d, J=6.8 Hz, 3H), 4.27 (d, J=13.8 Hz, 1H), 4.11 (d, J=12.8 Hz, 2H), 3.78 (d, J=13.6 Hz, 1H), 3.65 (d, J=13.2 Hz, 1H), 3.49-3.36 (m, 1H), 3.27 (d, J=11.6 Hz, 2H), 3.13 (s, 1H), 2.93 (dd, J=18.2, 4.4 Hz, 1H), 2.37-2.19 (m, 5H), 2.14-2.05 (m, 4H), 2.03-1.99 (m, 4H), 1.97-1.91 (m, 2H).

Compound 24-6D (30 mg, 30.00 μmol) was dissolved in methylene chloride (3 mL), then zinc bromide (14 mg, 30.00 μmol) was added, and the reaction was stirred at 25° C. for 2 hours under nitrogen protection. The reaction solution was concentrated under reduced pressure to obtain crude product. The crude product was prepared for high performance liquid chromatography separation (chromatographic column: Waters SunFire, 250*19 mm, 5 μm; mobile phase: [water (0.1% formic acid)-acetonitrile]; acetonitrile]: 10%-40%. After lyophilization, the formate of compound 24D was obtained. MS m/z=700.5 [M+H]⁺. ¹H NMR (400 MHz, CD₃OD) δ ppm 8.46 (s, 3H), 6.92 (d, J=8.4 Hz, 1H), 5.19 (dd, J=11.0, 4.0 Hz, 1H), 4.98-4.92 (m, 1H), 4.91-4.89 (m, 2H), 4.72 (d, J=13.6 Hz, 2H), 4.60-4.57 (m, 2H), 4.37-4.23 (m, 3H), 4.21-4.15 (m, 1H), 4.07 (d, J=12.6 Hz, 2H), 3.76 (d, J=14.6 Hz, 1H), 3.63 (d, J=13.6 Hz, 1H), 3.27 (d, J=13.2 Hz, 3H), 3.01-2.96 (m, 1H), 2.95-2.86 (m, 1H), 2.33-2.13 (m, 5H), 2.13-2.03 (m, 4H), 2.02 (s, 3H), 1.99-1.85 (m, 3H).

### Example 25

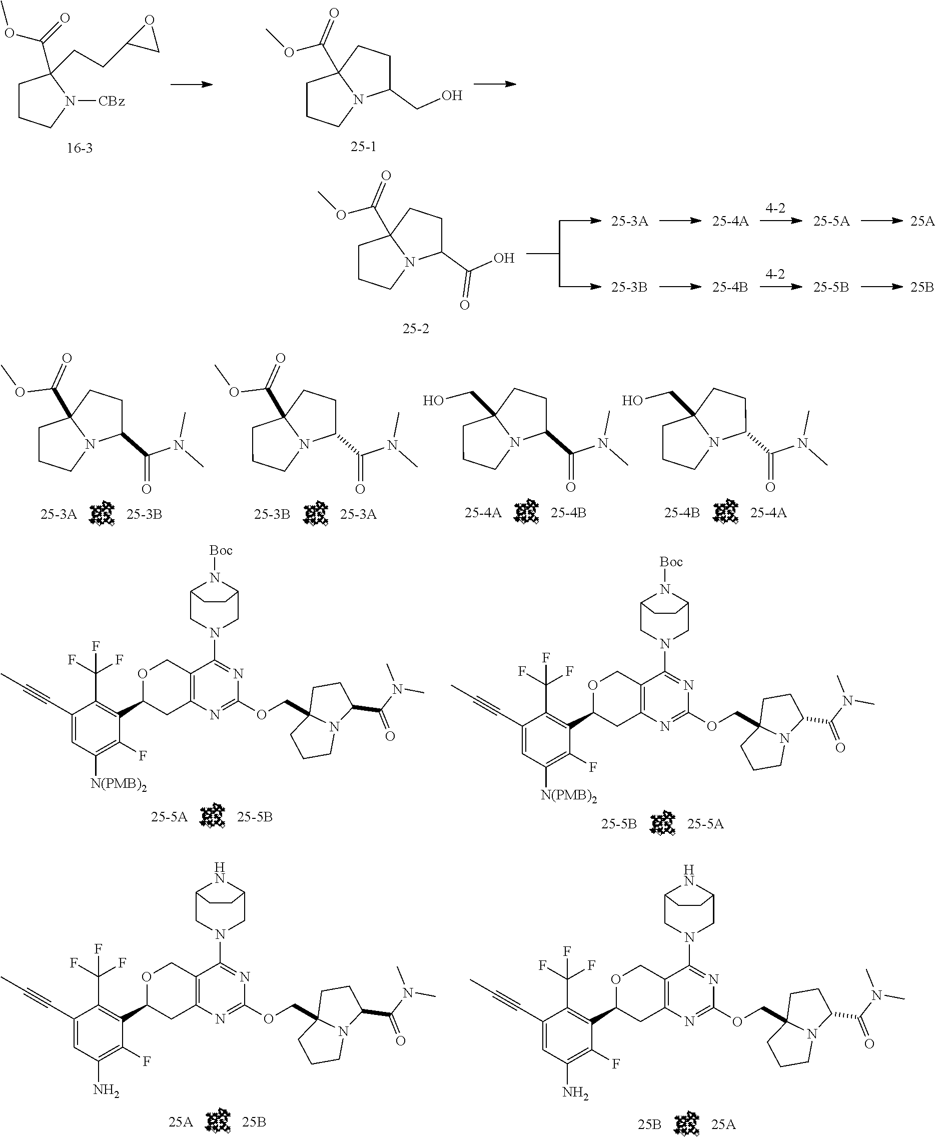

### Step 1

Compound 16-3 (8.00 g, 24.00 mmol) was dissolved in methanol (30 mL), 10% palladium carbon (1.5 g) was added, and the reaction mixture was stirred at 25° C. for 24 hours under hydrogen gas atmosphere, the reaction mixture was filtered and the filtrate was concentrated to obtain compound 25-1. MS m/z=200.2 [M+H]⁺.

### Step 2

Periodic acid (12.30 g, 53.95 mmol) was dissolved in an acetonitrile solution (0.075% water) (50.0 mL) solution, chromium trioxide (647.40 mg, 6.47 mmol) was slowly added at 0° C. and stirred for 10 minutes. Dissolve compound 25-1 in acetonitrile (50.0 mL), slowly add it to the above mixed solution at 0° C., add it to slowly warm to room temperature, stir for 10 hours, filter, and concentrate the filtrate under reduced pressure to obtain compound 25-2. MS m/z=214.2 [M+H]⁺.

### Step 3

Compound 25-2 (0.90 g, 4.22 mmol) was dissolved in N,N-dimethylformamide (20 mL), benzotriazole-N,N,N′,N′,-tetramethyl-uronium-hexafluorophosphate (2.41 g, 6.33 mmol) and N,N-diisopropylethylamine (2.73 g, 21.10 mmol) were added, and the reaction mixture was stirred at room temperature for 30 minutes. Dimethylamine hydrochloride (1.72 g, 21.10 mmol) was added to the above mixture and the resulting mixture was stirred at room temperature for 16 hours. The reaction mixture was diluted with water (100 mL) and extracted with dichloromethane (35 mL). The organic phase was washed with saturated brine (10 mL), dried with anhydrous sodium sulfate, filtered, concentrated under reduced pressure to obtain the crude product. The crude product was prepared for high performance liquid chromatography separation (chromatographic column: Waters Xbridge, 250*19 mm, 5 mm, mobile phase: [water (0.1% ammonia-acetonitrile]; gradient: (acetonitrile): 10%-40% to obtain compound 25-3A and compound 25-3B. By LCMS analytical method (chromatographic column. Waters Xbridge C18, (50 mm×4.6 mm×3.5 μm); mobile phase: [A: water (0.1% ammonia water) B: acetonitrile]; gradient: B %: 0-10% and 0.2 min; 20-95% 1.8 min, 95-95% 0.7 min; 95-10% 0.1 min; 10-10% 0.7 min) compound 25-3A Rt=1.780 min, MS m/z=241.2 [M+H]⁺, and compound 25-3B Rt=1.833 min, MS m/z=241.2 [M+H]⁺.

### Step 4

Compound 25-3A (0.13 g, 540.99 μmol) was dissolved in methanol (5 mL), and sodium borohydride (61.40 mg, 1.62 mmol) and 30% sodium methanol in methanol (2.92 mg, 54.10 μmol) were added at 0° C. The reaction mixture was stirred at 25° C. for 16 hours. After the reaction is completed, saturated ammonium chloride solution (10 ml) is added for quenching, dichloromethane (10 mL) is extracted, the organic phase is washed with saturated brine (5 mL), dried with anhydrous sodium sulfate, filtered, and concentrated under reduced pressure. Column chromatography separation (dichloromethane:methanol=10:1) yielded compound 25-4A. MS m/z=213.1 [M+H]⁺.

With reference to step 4, compound 25-3A was replaced with compound 25-3B as the raw material to obtain compound 25-4B. MS m/z=213.1 [M+H]⁺.

### Step 5

Compound 25-4A (50.00 mg, 235.53 μmol) was dissolved in tetrahydrofuran (2 mL) and then cooled to 0° C. NaH (16 96 mg, 706.59 μmol, 60% purity) was added and stirred for 1 hour. Compound 4-2 (50.00 mg, 235.53 μmol) was added to the above mixture and the resulting mixture was stirred and reacted at 25° C. for 1 hour. The resulting mixture was diluted with water (5 mL) and extracted with ethyl acetate (20 mL). The organic phases were washed with saturated brine (2 mL), dried over anhydrous sodium sulfate, filtered, and concentrated under reduced pressure. Column chromatography separation (dichloromethane:methanol=10:1) yielded compound 25-5A. MS m/z=1012.8 [M+H]⁺.

With reference to step 5, compound 25-4A was replaced with compound 25-4B as the raw material to obtain compound 25-5B. MS m/z=1012.8 [M+H]⁺.

### Step 6

Compound 25-5A (0.14 g, 138.32 μmol) was dissolved in trifluoroacetic acid (2 mL) and stirred and reacted at 25° C. for 1 hour. The reaction mixture was concentrated to remove TFA. Crude product was prepared for high performance liquid chromatography separation (chromatographic column: Waters Xbridge, 250*19 mm, 5 mm; [water (0.1% ammonia)-acetonitrile]; gradient: (acetonitrile): 30%-70% mobile phase: A water (0.1% ammonia water) and B (acetonitrile); gradient: B %=30%-70%, and compound 25A was obtained after lyophilization. MS m/z=672.6 [M+H]⁺. ¹H NMR (400 MHz, CD₃OD) δ ppm 6.90 (d, J=8.0 Hz, 1H), 5.13-5.09 (m, 1H), 4.78-4.67 (m, 2H), 4.54 (d, J=8.0 Hz, 1H), 4.35-4.28 (m, 1H), 4.26-4.23 (m, 1H), 4.13-4.09 (m, 1H), 3.91-3.88 (m, 1H), 3.27-3.19 (m, 2H), 3.16-3.10 (m, 4H), 2.94-2.88 (m, 4H), 2.79-2.66 (m, 4H), 2.22-2.15 (m, 2H), 2.11-1.89 (m, 11H), 1.78-1.75 (m, 1H), 1.72-1.66 (m, 1H).

Compound 25-5B (60.00 mg, 59.28 μmol) was dissolved in trifluoroacetic acid (1 mL) and stirred and reacted at 25° C. for 1 hour. The reaction mixture was concentrated to remove TFA. Crude product was prepared for high performance liquid chromatography separation (chromatographic column: Waters Xbridge, 250*19 mm, 5 mm; [water (0.1% ammonia)-acetonitrile]; gradient: (acetonitrile): 30%-70% mobile phase: A water (0.1% ammonia water) and B (acetonitrile); gradient: B %=30%-70%, and compound 25B was obtained after lyophilization. MS m/z=672.1 [M+H]⁺. ¹H NMR (400 MHz, CD₃OD) δ ppm 6.90 (d, J=8.0 Hz, 1H), 5.13-5.10 (m, 1H), 4.78-4.67 (m, 2H), 4.52-4.47 (m, 1H), 4.32-4.28 (m, 1H), 4.26-4.21 (m, 1H), 4.15-4.10 (m, 1H), 3.91-3.87 (m, 1H), 3.27-3.22 (m, 2H), 3.15-3.13 (m, 3H), 2.93-2.92 (m, 3H), 2.80-2.72 (m, 4H), 2.67-2.66 (m, 1H), 2.54-2.50 (m, 1H), 2.18-2.04 (m, 3H), 2.02-1.89 (m, 7H), 1.83-1.74 (m, 4H), 1.62-1.55 (m, 1H).

### Example 26

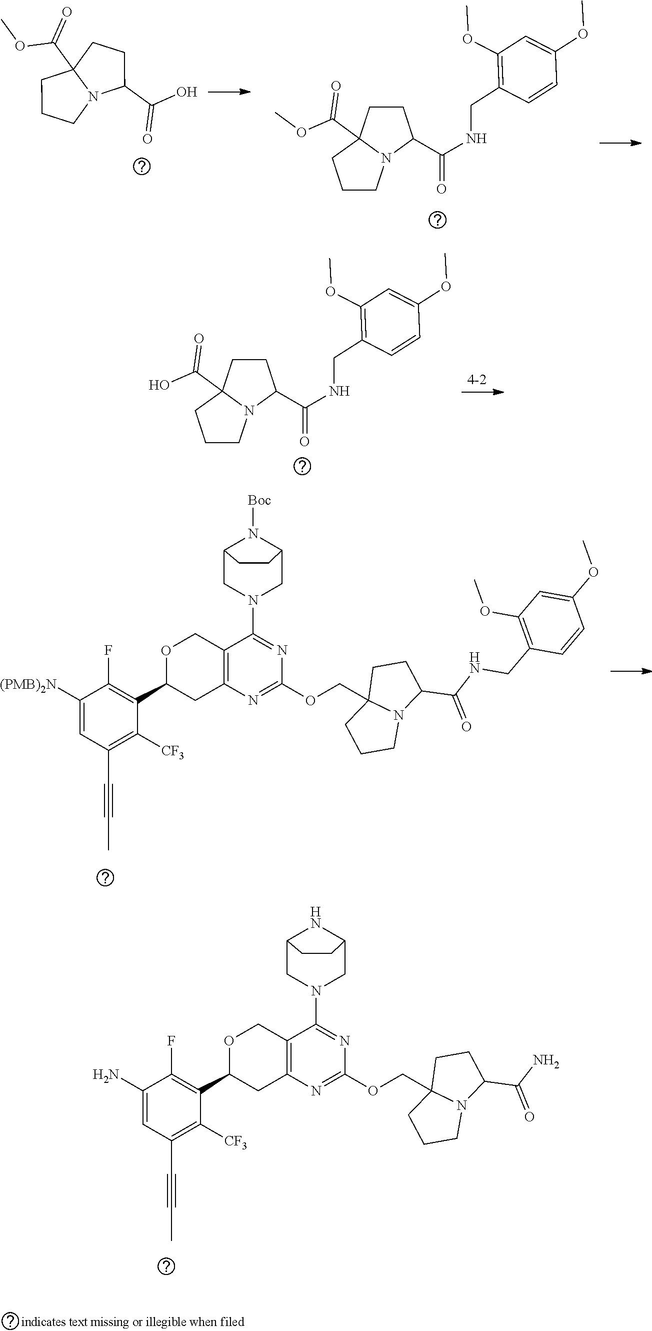

### Step 1

Compound 25-2 (1.00 g, 4.69 mmol) was dissolved in N, N-dimethylformamide (20 mL), followed by addition of N, N-diisopropylethylamine (2.12 g, 16.41 mmol), N,N,N′,N′-tetramethyl-O-(7-azabenzotriazole-1-yl) hexafluorourea phosphate (2.67 g, 7.03 mmol), 2, 4-dimethoxybenzoamine 1.02 g, 6.10 mmol) and reaction at 25° C. for 2 hours. The reaction solution was added with water (50 mL), extracted with ethyl acetate (40 mL*3), concentrated under reduced pressure, and column chromatography separated (petroleum ether:ethyl acetate=2:3) to obtain compound 26-1. MS m/z=363.5 [M+H]⁺.

### Step 2

Compound 26-1 (50.00 mg, 137.96 μmol) was dissolved in ethanol (1 mL of tetrahydrofuran (0.6 mL), lithium chloride (11.70 mg, 275.92 μmol), sodium borohydride (10.44 mg, 275.92 μmol), and reacted at 50° C. for 3 hours. Cool to room temperature, add DCM (20 mL), saturated saline (3 mL*3) wash, dry with anhydrous sodium sulfate, filter, and concentrate under reduced pressure to obtain compound 26-2. MS m/z=335.3 [M+H]⁺.

### Step 3

Compound 26-2 (30.00 mg, 89.71 μmol) was dissolved in tetrahydrofuran (3 mL), sodium tert-butanol (43.11 mg, 44.85 μmol) was added, stirred for 0.5 hour, and compound 4-277.51 mg, 89.71 μmol) was added finally, and reacted at 25° C. for 1 hour. Add water (3 mL) to quench the reaction, extract with ethyl acetate (20 mL), concentrate under reduced pressure, and separate by column chromatography (petroleum ether:ethyl acetate=1:4) to obtain compound 26-3. MS m/z=1134.6 [M+H]⁺.

### Step 4

Compound 26-3 (50.00 mg, 44.08 μmol) was dissolved in trifluoroacetic acid (1 mL) and reacted at 70° C. for 1 hour. The reaction solution was concentrated under reduced pressure to obtain crude product. The crude product was prepared for high performance liquid chromatography separation (chromatographic column: Waters SunFire, 250*19 mm, 5 μm; mobile phase: [water (0.1% formic acid)-acetonitrile]; gradient: (acetonitrile): 40%-75%. After lyophilization, the formate of compound 26 was obtained. MS m/z=644.1 [M+H]⁺. ¹H NMR (400 MHz, CD₃OD) δ ppm 8.38 (brs, 1H), 6.91 (d, J=8.5 Hz, 1H), 5.20-5.12 (m, 1H), 4.82-4.66 (m, 4H), 4.47 (d, J=6.4 Hz, 1H), 4.27 (q, J=10.8 Hz, 2H), 3.58 (d, J=12.5 Hz, 1H), 3.52-3.41 (m, 1H), 3.26-3.15 (m, 4H), 2.91-2.77 (m, 2H), 2.38-2.28 (m, 2H), 2.20-2.09 (m, 2H), 2.07-1.93 (m, 9H), 1.86-1.71 (m, 2H).

### Example 27

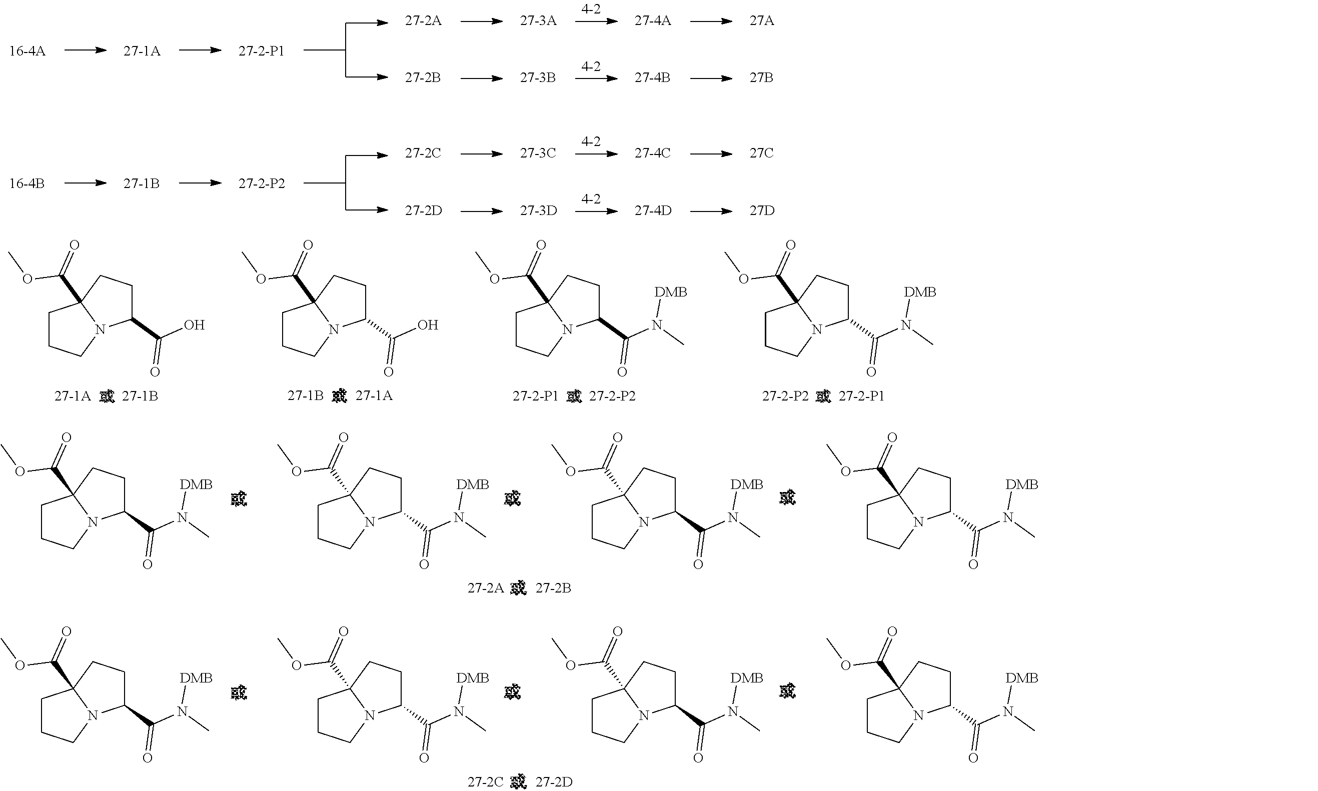

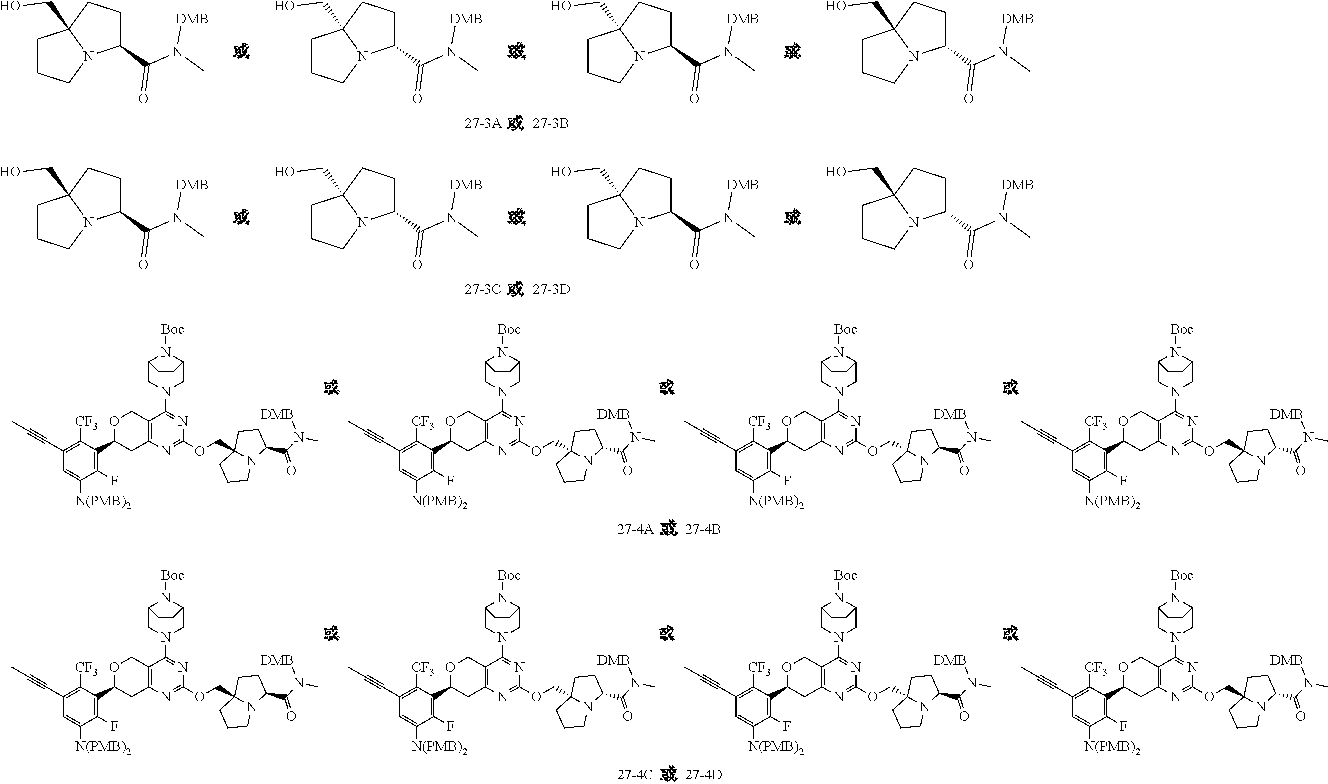

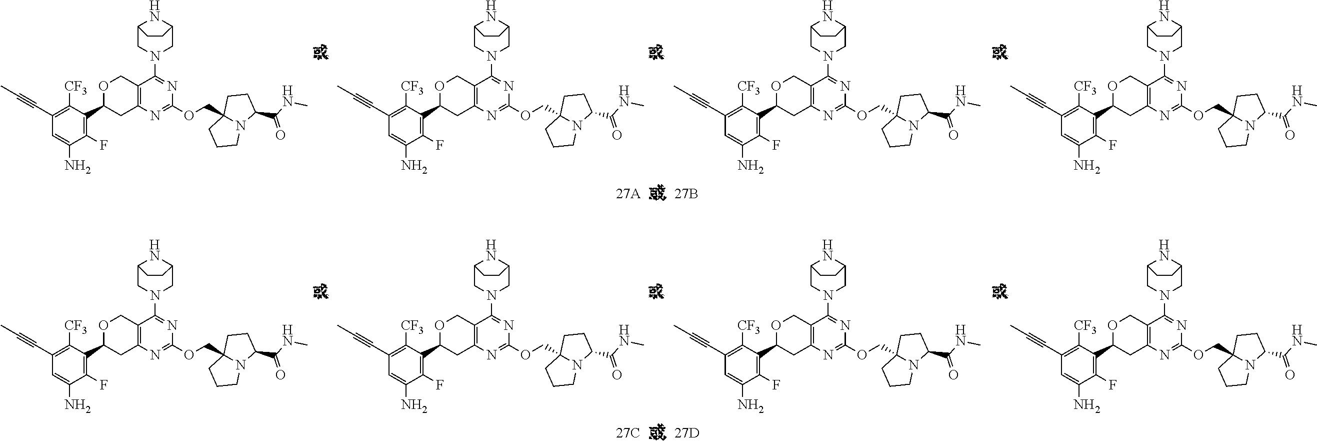

### Step 1

Dissolve periodine acid (1.71 g, 7.54 mmol) in 15 ml of mixed solvent (acetonitrile:Water=100:0.75), chromium trioxide (89.54 mg, 0.90 mmol) was added at 0° C., and stirred for 15 minutes. Compound 16-4A (0.60 g 3.02 mmol) was dissolved in acetonitrile (15 mL), slowly added to the above mixed solution at 0° C. After addition, the mixture was slowly heated to room temperature, stirred for 16 hours, the reaction solution was filtered, and the filtrate was concentrated under reduced pressure to obtain compound 27-1A, MS m/z=214.0 [M+H]⁺.

With reference to step 1, compound 16-4A was replaced with compound 16-4B to obtain compound 27-1B. MS m/z=214.0 [M+H]⁺

### Step 2

Compound 27-1A (0.20 g, 0.94 mmol) was dissolved in N. N-dimethylformamide (5 mL), followed by addition of 2,4-dimethoxy-N-methylaniline (254.92 mg 1.41 mmol), N, N-diisopropylethylamine (605.63 mg 4.69 mmol), 2-(7-azobenzotriazol)-N,N,N′,N′-tetramethylurea hexafluorophosphate (606.57 mg 1.60 mml) for 1 h at room temperature. Add 25 mL of water to the reaction solution, extract with ethyl acetate (15 mL*3), combine the organic phases, wash successively with water (10 mL*2) and saturated esophagus saline (10 mL), dry with anhydrous sodium sulfate, filter, and concentrate. Column chromatography (dichloromethane:methanol=20:1) yielded compound 27-2-P1, MS m/z=377.1 [M+H]⁺.

Compound 27-2-P1 was prepared for SFC separation (chromatographic column: DAICEL CHIRALPAK®AS-10, 25*250 mm 10 μm; mobile phase: A: supercritical carbon dioxide, B: [0.05% ammonia methanol-ethanol]; B %: 80%-20% to obtain compound 27-2A and compound 27-2B. After analysis SFC (column: DAICEL CHIRALPAK®AS-10 (4.6 mmI.D*150 mmL, 5 μm) mobile phase: [A: supercritical carbon dioxide, B: ethanol (0.05% diethylamine)]; gradient: B %: 5%-40%, 4 min), compound 27-2A Rt=2.824 min, ee value 99.05%, MS m/z=377.1 [M+H]; compound 27-2B Rt=3.327 min, ee 99.10%, MS m/z=377.1 [M+H]⁺.

With reference to step 2, compound 27-1A was replaced with compound 27-1B to obtain compound 27-2-P2. MS m/z=377.1 [M+H]⁺.

Compound 27-2-P2 was prepared for SFC separation (chromatographic column: DAICEL CHIRALPAK®AS-10, 25*250 mm 10 μm; mobile phase: A: supercritical carbon dioxide, B: [0.05% ammonia-methanol]; B %: 80%-20% to obtain compound 27-2C and compound 27-2D. After analysis, SFC (column: DAICEL CHIRALPAK®AS-10 (4.6 mmI.D*150 mmL, 5 μm) was analyzed; mobile phase: [A: supercritical carbon dioxide, B: ethanol (0.05% diethylamine)]; gradient: B %: 5%-40%, 4 min), compound 27-2C Rt=2.891 min, ee value 95.80%, MS m/z=377.1 [M+H]⁺; compound 27-2D Rt=3.327 min, ee value 96.34%, MS m/z=377.1 [M+H]⁺.

### Step 3

Compound 27-2A (80.00 mg, 0.21 mmol) was dissolved in methanol (5 mL), sodium methanol solution (7.66 mg, 0.04 mmol, 30% purity) and sodium borohydride (24.2 mg, 0.63 mmol) were added and stirred at 25° C. for 16 hours. 3 mL of saturated aqueous ammonium chloride was added for quenching, extracted with dichloromethane (5 mL*3), combined with the extracted organic phase, washed with 5 mL of saturated salt solution, dried with anhydrous sodium sulfate, filtered, concentrated under reduced pressure, and column chromatography separated (dichloromethane:ethyl acetate=20:1) to obtain compound 27-3A. MS m/z=349.1 [M+H]⁺.

With reference to step 3, compound 27-2A was replaced with compound 27-2B as the raw material to obtain compound 27-3B. MS m/z=349.1 [M+H]⁺.

With reference to step 3, compound 27-2A was replaced with compound 27-2C as the raw material to obtain compound 27-3C. MS m/z=349.1 [M+H]⁺.

With reference to step 3, compound 27-2A was replaced with compound 27-2D as the raw material to obtain compound 27-3D. MS m/z=349.1 [M+H]⁺.

### Step 4

Compound 27-3A (60.00 mg, 0.17 mmol) was dissolved in tetrahydrofuran (5 mL), sodium hydrogen (34.48 mg, 0.85 mmol, 60% purity) was added at 0° C., and the reaction was stirred for 0.5 hours. The solution of compound 4-2 (164.56 mg, 0.19 mmol) in tetrahydrofuran (1 mL) was then added to the above system and heated to 25° C. for continued stirring and reacting for 1 hour. The reaction was quenched by adding 6 mL of saturated aqueous ammonium chloride solution, extracted with ethyl acetate (5 mL*2), combined with the extracted organic phase, washed with 5 mL of saturated brine, dried with anhydrous sodium sulfate, filtered, concentrated under reduced pressure, and column chromatography separated (dichloromethane:methanol=20:1) to obtain compound 27-4A. MS m/z=1148.5 [M+H]⁺.

With reference to step 4, compound 27-3A was replaced with compound 27-3B as the raw material to obtain compound 27-4B. MS m/z=1148.5 [M+H]⁺.

With reference to step 4, compound 27-3A was replaced with compound 27-3C as the raw material to obtain compound 27-4C. MS m/z=1148.5 [M+H]⁺.

With reference to step 4, compound 27-3A was replaced with compound 27-3D as the raw material to obtain compound 27-4D. MS m/z=1148.5 [M+H]⁺.

### Step 5

Compound 27-4A (0.10 g, 87.15 μmol) was dissolved in trifluoroacetic acid (3 mL), and the reaction was stirred and reacted at 25° C. for 1 hour. The reaction solution was concentrated under reduced pressure to obtain crude product. Preparation of crude product for high performance liquid chromatography separation (chromatographic column: Waters Xbridge, 250*19 mm, 5 μm; mobile phase: [water (0.1% ammonia water)-acetonitrile]; acetonitrile). 50%-70%), the compound 27A was obtained after lyophilization. MS m/z=658.6 [M+H]⁺. ¹H NMR (400 MHZ, CD₃OD) δ ppm 6.90 (d, J=8.0 Hz, 1H), 5.18-5.10 (m, 1H), 4.78 (s, 1H), 4.65 (d, J=12.0 Hz, 1H), 4.23 (d, J=12.0 Hz, 1H), 4.16-4.07 (m, 2H), 3.57-3.48 (m. 3H), 3.45-3.38 (m, 1H), 3.29-3.17 (m, 2H), 3.10-3.01 (m, 2H), 2.89-2.81 (m, 1H), 2.73 (s, 3H), 2.67-2.60 (m, 1H), 2.31-2.23 (m, 1H), 2.15-2.08 (m, 1H), 2.04-1.96 (m, 4H), 1.95-1.87 (m, 4H), 1.86-1.79 (m, 2H), 1.76-1.65 (m, 3H).

Compound 27-4B (80.00 mg, 69.71 μmol) was dissolved in trifluoroacetic acid (3 mL), and the reaction was stirred and reacted at 25° C. for 1 hour. The reaction solution was concentrated under reduced pressure to obtain crude product. The crude product was prepared for high performance liquid chromatography separation (chromatographic column: Waters Xbridge, 250*19 mm, 5 μm; mobile phase: [water (0.1% ammonia water)-acetonitrile]; acetonitrile): 50%-70%), and compound 27B was obtained after lyophilization. MS m/z=658.5 [M+H]′. ¹H NMR (400 MHZ, CD₃OD) δ ppm 6.90 (d, J=8.0 Hz, 1H), 5.21-5.09 (m, 1H), 4.80 (d, J=12.0 Hz, 1H), 4.65 (d, J=12.0 Hz, 1H), 4.25 (d, J=9.0 Hz, 1H), 4.16-4.06 (m, 2H), 3.57-3.48 (m, 3H), 3.44-3.38 (m, 1H), 3.30-3.15 (m, 2H), 3.10-3.01 (m, 2H), 2.89-2.80 (m, 1H), 2.73 (s, 3H), 2.68-2.60 (m, 1H), 2.31-2.21 (m, 1H), 2.19-2.09 (m, 1H), 2.01-1.96 (m, 4H), 1.94-1.78 (m, 6H), 1.77-1.64 (m, 3H).

Compound 27-4C (90.00 mg, 78.36 μmol) was dissolved in trifluoroacetic acid (3 mL), and the reaction was stirred and reacted at 25° C. for 1 hour. The reaction solution was concentrated under reduced pressure to obtain crude product. Preparation of crude product for high performance liquid chromatography separation (chromatographic column: Waters Xbridge, 250*19 mm, 5 μm; mobile phase: [water (0.1% ammonia water)-acetonitrile]; acetonitrile): 50%-70%). After lyophilization, compound 27C was obtained. MS m/z=658.3 [M+H]. 1H NMR (400 MHZ, CD₃OD) δ ppm 6.90 (d, J=8.0 Hz, 1H), 5.19-5.10 (m, 1H), 4.79 (d, J=12.0 Hz, 1H), 4.65 (d, J=12.0 Hz, 1H), 4.18-4.10 (m. 2H), 4.10-4.05 (m, 1H), 3.76-3.71 (m, 1H), 3.57-3.48 (m, 3H), 3.45-3.38 (m, 1H), 3.26-3.16 (m, 1H), 3.04 (d, J=12.7 Hz, 1H), 2.92-2.81 (m, 2H), 2.75 (s, 3H), 2.75-2.68 (m, 1H), 2.14-2.05 (m, 2H), 2.04-1.97 (m, 5H), 1.96-1.80 (m, 6H), 1.76-1.64 (m, 2H).

Compound 27-4D (95.00 mg, 82.28 μmol) was dissolved in trifluoroacetic acid (3 mL), and the reaction was stirred and reacted at 25° C. for 1 hour. The reaction solution was concentrated under reduced pressure to obtain crude product. The crude product was prepared by high performance liquid chromatography separation (chromatographic column: Waters Xbridge, 250*19 mm, 5 μm; mobile phase: [water (0.1% ammonia water)-acetonitrile]; acetonitrile): 50%-70%). After lyophilization, compound 27D was obtained. MS m/z=658.4 [M+H]⁺. ¹H NMR (400 MHZ, CD₃OD) δ ppm 6.90 (d, J=8.0 Hz, 1H), 5.18-5.11 (m, 1H), 4.79 (d, J=12.0 Hz, 1H), 4.64 (d, J=12.0 Hz, 1H), 4.19-4.10 (m, 2H), 4.07-4.01 (m, 1H), 3.76-3.71 (m, 1H), 3.56-3.48 (m. 3H), 3.45-3.38 (m, 1H), 3.26-3.17 (m, 1H), 3.03 (d,/=12.6 Hz, 1H), 2.92-2.81 (m, 2H), 2.75 (s, 3H), 2.75-2.69 (m, 1H), 2.15-2.06 (m, 2H), 2.05-1.97 (m, 5H), 1.96-1.80 (m, 6H), 1.76-1.65 (m, 2H).

### Example 28

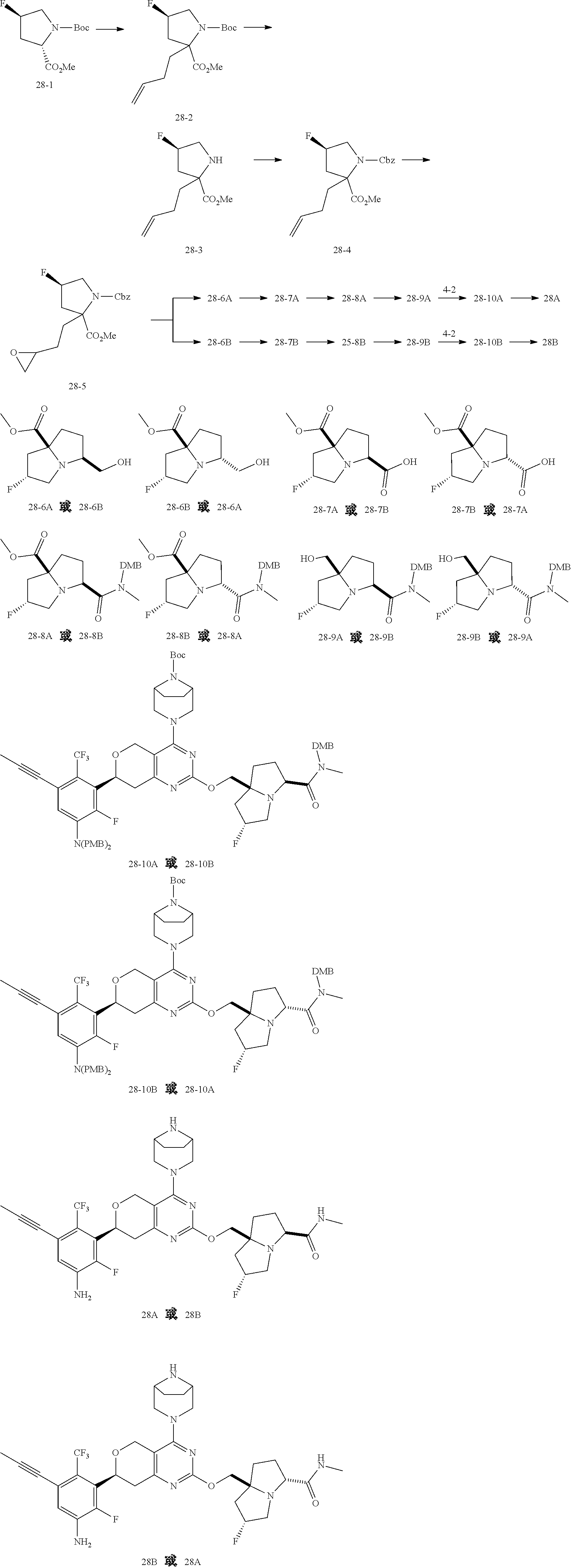

### Step 1

Weigh compound 28-1 (1 g, 4.04 mmol), add anhydrous tetrahydrofuran (10 mL) under nitrogen protection, carry out dry ice bath to −76° C., slowly add lithium bis(trimethylsilyl)amide (12.12 mL, 12.12 mmol, 1M), stir at −76° C. for 40 minutes, add 4-bromobutene (654.48 mg, 4.84 mmol) in anhydrous tetrahydrofuran solution (5 mL), continue stirring for 60 minutes. The reaction was quenched by adding saturated aqueous ammonium chloride solution (10 mL), extracted with ethyl acetate (15 mL*3), combined with the extracted organic phase, washed with saturated brine (20 mL), dried with anhydrous sodium sulfate, filtered, and concentrated under reduced pressure. Column chromatography separation (ethyl acetate:petroleum ether=1:7) yielded compound 28-2. MS m/z=302.2 [M+H]⁺

### Step 2

Weigh compound 28-2 (500 mg, 1.66 mmol), add dichloromethane (3 mL), hydrochloric acid/1,4-dioxane solution (3 mL), and react at room temperature for 30 minutes. The reaction solution was directly concentrated to obtain the hydrochloride of compound 28-3, MS m/z=202.1 [M+H]⁺

### Step 3

Weigh compound 28-3 (500 mg, hydrochloride), add anhydrous tetrahydrofuran (10 mL), add sodium hydride (148.80 mg, 3.72 mmol, 60% purity) under ice bath at 0° C., stir at 0° C. for 1 hour, add benzyl chloroformate (634.59 mg, 3.72 mmol) in batches, and stir at 40° C. for 16 hours. The reaction was quenched with water (15 mL), extracted with ethyl acetate (20 mL*3), combined with the extracted organic phase, washed with saturated brine (20 mL), dried with anhydrous sodium sulfate, filtered, and concentrated under reduced pressure. Column chromatography separation (ethyl acetate: Petroleum ether=1:8) obtained compound 28-4, MS m/z=336.2 [M+H]⁺.

### Step 4

Weigh compound 28-4 (300 mg, 0.89 mmol), add methylene chloride (5 mL), add m-chloroperoxybenzoic acid (307.17 mg, 1.78 mmol), and stir and reacted at room temperature for 2 hours. Add saturated sodium sulphite aqueous solution (1 mL) to the reaction solution, stir for 5 minutes, add water (10 mL), add methylene chloride (15 mL*3) for extraction, combine the extracted organic phases, wash with saturated edible brine (20 mL), dry with anhydrous sodium sulfate, filter, and concentrate under reduced pressure. Column chromatography separation (ethyl acetate Petroleum ether=1:6) to obtain compound 28-5, MS m/z=352.2 [M+H]⁺.

### Step 5

Weigh compound 28-5 (1 g, 2.85 mmol), add anhydrous methanol (10 mL), add 10% palladium carbon (100 mg), and replace with hydrogen gas three times. Stir at room temperature for 1 hour. The reaction solution was directly filtered, the cake was washed with methanol (10 mL), and the filtrate was concentrated under reduced pressure. Column chromatography separation (dichloromethane:methanol=50:1) yielded compound 28-6A (unfolding agent: DCM/MeOH=15:1, Rf=0.5), MS m/z=218.1 [M+H]⁺, and compound 28-6B (unfolding agent: DCM/MeOH=15:1, Rf=0.3 MS m/z=218.1 [M+H]⁺

Compound 28-6A: ¹H NMR (400 MHZ, DMSO-d6) δ ppm 5.39-5.21 (m, 1H), 4.37 (d, J=8.0 Hz, 1H), 3.58 (s, 3H), 3.39-3.35 (m, 1H), 3.29-3.21 (m, 2H), 2.99-2.86 (m, 1H), 2.82-2.77 (m, 1H), 2.61-2.52 (m, 1H), 2.09-1.91 (m, 3H), 1.84-1.78 (m, 1H), 1.68-1.60 (m, 1H).

Compound 28-6B: ¹H NMR (400 MHZ, DMSO-d6) δ ppm 5.27-5.10 (m, 1H), 4.64 (d, J=8.0 Hz, 1H), 3.59 (s, 3H), 3.58-3.54 (m, 1H), 3.51-3.46 (m, 1H), 3.25-3.18 (m, 1H), 3.09-2.95 (m, 2H), 2.69-2.60 (m, 1H), 2.02-1.70 (m, 4H), 1.59-1.48 (m, 1H).

### Step 6

Compound 28-6A (200.00 mg, 920.65 mmol) was weighed, acetonitrile (2 mL) and water (0.014 mL) were added, and the temperature was reduced to 0° C. Periodic acid (662.87 mg, 2.30 mmol) was added, chromium trioxide (27.62 mg, 1.84 mmol) was added, and the reaction was performed at room temperature for 4 hours. The reaction solution was filtered, the filter cake was washed with 10 mL of dichloromethane, and the filtrate was concentrated to obtain compound 28-7A, MS m/z=232.1 [M+H]⁺.

With reference to step 6, compound 28-6A was replaced with compound 28-6B as the raw material to obtain compound 28-7B. MS m/z=232.1 [M+H]⁺.

### Step 7

Compound 28-7A (120 mg, 518.99 μmol) was dissolved in N, N-dimethylformamide (3 mL), N-methyl-3,4-dimethylbenzylamine (92.94 mg, 622.79 μmol), 2-(7-azabenzotriazazole)-N,N,N′,N′-tetramethyluronium hexafluorophosphate (394.67 mg, 1.04 mmol), N,N-diisopropylethylamine (201.22 mg, 1.56 mmol) was added and reacted at 25° C. for 4 hours. Extracted with ethyl acetate (20 mL*2), combined with the extracted organic phase, washed with saturated brine (20 mL), dried over anhydrous sodium sulfate, filtered, concentrated under reduced pressure. Column chromatography separation (petroleum ether: Ethyl acetate=10:1-1:1) to obtain compound 28-8A. MS m/z=395.2 [M+H]⁺.

With reference to step 7, compound 28-7A was replaced with compound 28-7B as the raw material to obtain compound 28-8B. MS m/z=395.2 [M+H]⁺.

### Step 8

Compound 28-8A (20.00 mg, 55.18 μmol) was dissolved in methanol (0.5 mL), sodium borohydride (4.18 mg, 110.36 μmol) was added, and sodium methanol (29.81 μg, 0.55 μmol) was added. React at 25° C. for 4 hours. Extract with ethyl acetate (20 mL*2), combine the extracted organic phases, wash with saturated brine (20 mL), dry with anhydrous sodium sulfate, filter, and concentrate under reduced pressure to obtain compound 28-9A. MS m/z=367.3 [M+H]⁺.

With reference to step 8, compound 28-8A was replaced with compound 28-8B as the raw material to obtain compound 28-9B. MS m/z=367.3 [M+H]⁺.

### Step 9

Compound 28-9A (20 mg, 54.58 μmol) was dissolved in anhydrous tetrahydrofuran (0.5 mL), cooled to 0° C., sodium hydrogen (2.62 mg, 109.16 μmol, 60% purity) was added under nitrogen protection, and reacted at room temperature for 1 hour. The reaction was quenched by adding 5 mL of saturated ammonium chloride solution, extracted with ethyl acetate (10 mL*2), combined with the extracted organic phase, washed with saturated brine (10 mL), dried with anhydrous sodium sulfate, filtered, and concentrated under reduced pressure. Preparation thin layer chromatography purification (dichloromethane:methanol=20:1) was prepared to obtain compound 28-10A. MS m/z=1166.5 [M+H]⁺.

With reference to step 9, compound 28-9A was replaced with compound 28-9B as the raw material to obtain compound 28-10B. MS m/z=1166.5 [M+H]⁺.

### Step 10

Compound 28-10A (20 mg, 17.63 μmol) was added to dichloromethane (1 mL) and trifluoroacetic acid (0.1 mL), and the solution was stirred and reacted at 20° C. for 2 hours. The reaction solution was concentrated under reduced pressure to obtain crude product. Crude product was prepared for high performance liquid chromatography separation (chromatographic column: Xtimate C18 150*40 mm*5 μm; mobile phase: [water (0.05% formic acid)-acetonitrile]; gradient: (acetonitrile): 10%-40%); after lyophilization, the formate of compound 28A was obtained MS m/z=676.5 [M+H]⁺. ¹H NMR (400 MHZ, CD: OD) δ ppm 8.52 (brs, 1H), 6.91 (d, J=8.0 Hz, 1H), 5.49-5.27 (m, 1H), 5.17 (dd, J=11.6, 4.5 Hz, 1H), 4.82-4.79 (m, 1H), 4.69 (d, J=16.0 Hz, 1H), 4.39 (d, J=12.0 Hz, 1H), 4.30-4.19 (m, 2H), 3.91 (s, 2H), 3.71-3.64 (m, 1H), 3.59-3.42 (m, 2H), 3.29-3.12 (m, 3H), 3.04-2.85 (m, 2H), 2.73 (s, 3H), 2.44-2.29 (m, 2H), 2.23-2.15 (m, 2H), 2.07-1.98 (m, 6H), 1.97-1.75 (m, 3H).

Compound 28-10B (20 mg, 17.63 μmol) was added to dichloromethane (1 mL) and trifluoroacetic acid (0.1 mL), and the solution was stirred and reacted at 20° C. for 2 hours. The reaction solution was concentrated under reduced pressure to obtain crude product. Crude product was prepared for high performance liquid chromatography separation (chromatographic column: Xtimate C18 150*40 mm*5 μm; mobile phase: [water (0.05% hydrochloric acid)-acetonitrile]; gradient: (acetonitrile: 10%-40%. After lyophilization, the hydrochloride of compound 28B is obtained. MS m/z=676.5 [M+H]. 1H NMR (400 MHZ, CD₃OD) δ ppm 6.90 (d, J=8.0 Hz, 1H), 5.42-5.25 (m, 1H), 5.18-5.11 (m, 1H), 4.79 (d, J=13.5 Hz, 1H), 4.64 (d, J=16.0 Hz, 1H), 4.23 (d, J=8.0 Hz, 1H), 4.20-4.11 (m, 2H), 3.75 (t, J=5.6 Hz, 1H), 3.52 (d, J=12.0 Hz, 3H), 3.42 (d, J=12.0 Hz, 1H), 3.26-3.12 (m, 2H), 3.10-2.99 (m, 2H), 2.88-2.80 (m, 1H), 2.73 (s, 3H), 2.45-2.35 (m, 1H), 2.24-2.07 (m, 2H), 2.06-1.96 (m, 7H), 1.87-1.77 (m, 2H), 1.68 (t, J=8.9 Hz, 1H)

### Biological Test Data

### Experimental Example 1. AsPC-1 Cell p-ERK Inhibition Test

### 1. Purpose:

The inhibitory activity of the compound on p-ERK levels of KRAS^{G12D}-mutant pancreatic cancer AsPC-1 cells was assessed by HTRF.

### 2. Experimental Materials:

ASPC-1 cells were purchased from ATCC; RPMI-1640 medium was purchased from Gibco; Fetal Bovine Serum was purchased from Hyclone; Advanced Phospho-ERK1/2 (THR202/TYR204) KIT was purchased from Bioauxilium—

TABLE B-1
Advanced Phospho-ERK1/2(THR202/TYR204)
KIT Component Table

| | Storage |
|---|---|
| Component Name | Temperature |
| Advanced PhosphoERK1/2 Eu Cryptate antibody | ≤−16° C. |
| Advanced PhosphoERK1/2 d2 antibody | ≤−16° C. |
| Blocking reagent (stock solution 100×) | ≤−16° C. |
| Lysis buffer # 1 (stock solution 4×) | ≤−16° C. |
| Detection buffer (ready-to-use) | ≤−16° C. |

### 3. Experimental Method:

ASPC-1 cells were seeded in a 384-well cell culture plate with a white bottom, 8 μL of cell suspension per well, containing 7,500 cells per well, and the cell plates were placed in a carbon dioxide incubator and incubated at 37° C. overnight;

The test compound was diluted to 3 mM with 100% DMSO as the first concentration, and then diluted to ten concentrations of 3000, 1000, 300, 100, 30, 10, 3, 1, 0.3, 0.1 μM with a pipette. Take 2 μL of compound and add 198 μL of cell starvation medium. After mixing well, take 15 μL of compound solution and add 35 μL of cell starvation medium and mix well. Then add 4 μL of compound solution in each well to the corresponding cell plate well. Place the cell plate back in the carbon dioxide incubator for continued incubation for 3 hours. At this time, the compound concentration is 3,000, 1,000, 300, 100, 30, 10, 3, 1, 0.3, 0.1 nM.

After the end of incubation, add 3 μL of 5× cell lysate to each well, and incubate at room temperature for 30 minutes;

Dilute Phospho-ERK1/2 Eu Cryptate antibody and Phospho-ERK1/2 d2 antibody 20-fold by using Detection buffer, mix well in a 1:1 ratio, add 5 μL to the cell culture plate per well, and incubate at room temperature for 2 h.

After the end of incubation, use a multi-label analyzer to read HTRF excitation: 320 nm, emission: 615 nm, 665 nm;

### 4. Data Analysis:

The raw data was converted to inhibition rate by applying the equation (Sample-Min)/(Max-Min)*100%, and the value of IC50 can be obtained by curve fitting with four parameters log (inhibitor) vs. response—Variable slope Surf mode in GraphPad Prism. Table B-2 provides the inhibition of p-ERK by the compounds of the present invention.
Max well: Positive control well read value is 1× lysate
Min well: Negative control well reads 0.5% DMSO well cell lysate

### 5. Experimental Results:

See Table B-2 for the results.

TABLE B-2
IC₅₀ values of compound inhibition on p-ERK of AsPC-1 cells

| Compound Number | AsPC-1 p-ERK IC₅₀ (nM) |
|---|---|
| Hydrochloride of Compound 1 | 164 |
| Trifluoroacetate of Compound 3A | 28.9 |

Experimental conclusion: The compounds of the present invention have a significant inhibitory effect on the p-ERK of KRAS^{G12D} mutant AsPC-1 cells.

### Experimental Example 2. AsPC-1 Cell p-ERK Inhibition Test

### Experimental Materials:

1640 medium, penicillin/streptomycin antibiotics were purchased from Gibco, and fetal bovine serum was purchased from Biosera. CellTiter-Glo (Cell Viability Chemiluminescence Detection Reagent) reagent was purchased from Promega. AsPC-1 cell line was purchased from ATCC, Envision Multi-Tag Analyzer (PerkinElmer).

### Experimental Method:

AsPC-1 (KRAS^{G12D} mutation, pancreatic cancer) cells were seeded in ultra-low adsorption 96-well blackwall plate, 80 μL cell suspension per well, containing 10,000 AsPC-1 cell lines from ATCC cells. Plates were incubated overnight in a carbon dioxide incubator.

The test compound was diluted 8 concentrations with a 5-fold drain, i.e., from 2 mM to 25.6 nM, and a double replicate well experiment was set up. Add 78 μL of culture medium to the intermediate plate, and transfer 2 μL of gradient diluted compound per well to the intermediate plate according to the corresponding positions. Mix well and transfer 20 μL of each well to the cell plate. Compound concentrations transferred to the cell plate ranged from 10 μM to 0.128 nM. The cell plates were incubated in a carbon dioxide incubator for 10 days. Another cell plate was prepared, and the signal value was read as the maximum value (Max value in the equation below) on the day of dosing to participate in the data analysis.

Each 100 μL of cell viability chemiluminescence detection reagent was added to the cell plate and incubated at room temperature for 30 minutes to stabilize the luminescence signal. Use multi-label analyzer for reading.

### Data Analysis:

The raw data was converted to inhibition rate by applying the equation (Sample-Min)/(Max-Min)*100%, and the value of IC50 can be obtained by curve fitting with four parameters “log (inhibitor) vs. response—Variable slope” Surf mode in GraphPad Prism. Table B-3 provides the inhibitory activity of the compound of the present invention on pERK levels in AsPC-1 cells.

TABLE B-3
IC₅₀ values of compound inhibition on p-ERK in AsPC-1 cells

| Compound Number | AsPC-1 p-ERK IC₅₀ (nM) |
|---|---|
| Trifluoroacetate of Compound SA | 7.4 |
| Trifluoroacetate of Compound 7 | 24.9 |
| Trifluoroacetate of Compound 12 | 35.0 |

Experimental conclusion: The compounds of the present invention have a significant inhibitory effect on the p-ERK of KRAS^{G12D} mutant AsPC-1 cells.

### Experimental Example 3. AsPC-1, Panc0403 Cell p-ERK Inhibition Test

### Experimental Method:

On the first day, 6000/well APC-1 (ATCC, CRL-1682, KRAS^{G12D} mutation, pancreatic cancer) or PANC04.03 (ATCC, CRL-2555, KRAS^{G12D} mutation, pancreatic cancer) were incubated in 384-well plates (Corning, 354663) overnight at 37° C. in an incubator with 5% CO₂. The next day, serially diluted compounds were added to cell plates (DMSO content of 0.1%) with echo 655, incubated at 37° C. with 5% CO₂ for 1.5 hours. Add 40 μL of 8% cell tissue fixative and fix at room temperature for 20 minutes. Discard the supernatant and wash twice with PBS for two minutes each. Add 40 μL of methanol and permeabilize at room temperature for 10 minutes. Discard the supernatant and wash twice with PBS for two minutes each. Add 20 μL of fixative and block at room temperature for 1 hour. Discard the supernatant and add 20 μL of prepared primary antibody diluent (1:1000-diluted Phospho-p44/42 MAPK (Erk1/2) (Thr202/Tyr204) (D13.14.4E) XP® Rabbit mAb and 1:2000 diluted GAPDH (D4C₆R) Mouse mAb), incubated overnight in a 4° C. refrigerator. On the third day, the primary antibody was discarded and washed 3 times with 1×PBST for 3 minutes each. Add the prepared secondary antibody diluent (1:2000 diluted IRDye 800CW Goat anti-Rabbit IgG (H+L) (0.5 mg) and 1:2000 diluted IRDye 680RD Goat anti Mouse IgG (H+L) (0.5 mg)) and incubated at room temperature for 1 hour away from light. The secondary antibody was discarded and washed 3 times with 1×PBST for 3 minutes each. The pERK and GAPDH signals were quantified at 800 nM and 700 nM, respectively, by using a Li-Cor Odyssey machine after 1 minute of inversion 10000 centrifugation.

### Experimental Results

See Table B-4 for the results.

TABLE B-4
IC₅₀ values of compound inhibition on
p-ERK of AsPC-1 and Panc0403 cells

| | AsPC-1 p-ERK | Panc0403 p-ERK |
|---|---|---|
| Compound Number | IC₅₀ (nM) | IC₅₀ (nM) |
| Trifluoroacetate of | 5.6 | 9.0 |
| Compound 4B | | |
| Trifluoroacetate of | 12 | 21.1 |
| Compound 9B | | |
| Trifluoroacetate of | 9.3 | 19.7 |
| Compound 10 | | |
| Trifluoroacetate of | 6.1 | 7.8 |
| Compound 13A | | |
| Trifluoroacetate of | 19.4 | 23.9 |
| Compound 14B | | |
| Trifluoroacetate of | 5.3 | 5.0 |
| Compound 15B | | |
| Compound 16D | 1.9 | 4.5 |
| Compound 17D | 22.4 | 23.8 |
| Compound 18D | 3.2 | 6.9 |
| Formate of Compound 19D | 1.1 | 3.6 |
| Compound 20D | 0.4 | 1 |
| Formate of Compound 21D | 10.9 | 13.7 |
| Compound 22D | 1.3 | 4.2 |
| Compound 23D | 0.6 | 2.1 |
| Formate of Compound 24D | 4.7 | 8.2 |
| Compound 25B | 110 | 121 |
| Compound 27D | 6.2 | 12.6 |
| Hydrochloride of Compound | 35.1 | 33.3 |
| 28B | | |

Experimental conclusion: The compounds of the present invention have a significant inhibitory effect on the p-ERK of KRAS^{G12D} mutant AsPC-1 and Panc0403 cells.

### Experimental Example 4. AsPC-1, Panc0403 Cell Proliferation Inhibition Test

### Experimental Method:

ASPC-1 (ATCC, CRL-1682, KRAS^{G12D} mutation, pancreatic cancer) and PANC04.03 (ATCC, CRL-2555, KRAS^{G12D} mutation, pancreatic cancer) cells were inoculated into CellCarrier-96 ULA/CS plate (PE, 6055330) at a density of 2,000 cells/well/195 μL, and the diluted compound was added to the cell plate and cultured in a 37° C. cell incubator for 7 days. CellTiter-Glo® 3D Detection Kit (3D-CTG, Promega, G9683) was left with cells at room temperature for 30 min, an equal volume of CTG was added to the assay plate and shaken for 20 min for cell lysis; cells were left at room temperature for 1 h and the luminescent signal was read using BMG (PHERAstar FSX). The percent compound viability of the treated wells was calculated between the control and blank groups. Wells containing cell culture medium were used as blank groups, and wells containing cells and the same percentage of DMSO were used as control groups.

Inhibition rate=(control luminescent reading−compound treated well luminescent reading)/(control luminescent reading-blank luminescent reading)*100%; then data were analyzed by fitting a 4-parameter logic model or Excel to calculate IC₅₀ values, which were concentrations at 50% inhibition on the curve.

### Experimental Results

See Table B-5 for the results.

TABLE B-5
IC₅₀ values of compound inhibition of
AsPC-1 and Panc0403 cell proliferation

| | AsPC-1 | Panc0403 |
|---|---|---|
| Compound Number | IC₅₀ (nM) | IC₅₀ (nM) |
| Trifluoroacetate of | 34.3 | 295 |
| Compound 4B | | |
| Trifluoroacetate of | 48.5 | 409 |
| Compound 9B | | |
| Trifluoroacetate of | 55.7 | 415 |
| Compound 10 | | |
| Trifluoroacetate of | 20.7 | 142 |
| Compound 13A | | |
| Trifluoroacetate of | 96.5 | 414 |
| Compound 14B | | |
| Trifluoroacetate of | 6.0 | 97 |
| Compound 15B | | |
| Compound 16D | 2.3 | 15.5 |
| Compound 17D | 67.3 | 644 |
| Compound 18D | 2.8 | 12.6 |
| Formate of Compound 19D | 2.9 | 7.9 |
| Compound 20D | 0.7 | 3.7 |
| Formate of Compound 21D | 2.1 | 6.8 |
| Compound 22D | 3.4 | 14.1 |
| Compound 23D | 1.7 | 5.3 |
| Formate of Compound 24D | 6.3 | 54 |
| Compound 25B | 124 | |
| Compound 27D | 8.7 | 71.4 |
| Hydrochloride of Compound | 69.8 | 124 |
| 28B | | |

Experimental conclusion: The compounds of the present invention have significant anti-proliferative inhibitory effects on KRAS^{G12D} mutant AsPC-1 and Panc0403 cells.

1. A compound of Formula (I″) or a stereoisomer thereof, or a pharmaceutically acceptable salt thereof: 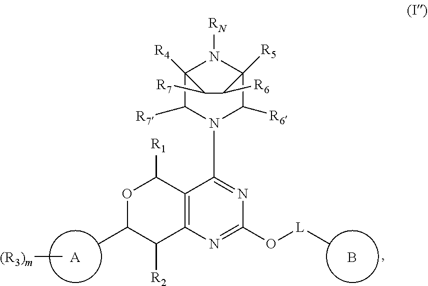
wherein,
R_{N} is selected from H and C₁₋₃ alkyl, wherein the C₁₋₃ alkyl is optionally substituted with 1, 2, or 3 F or Cl;
Ring A is selected from C₆ aryl and 5-6 membered heteroaryl;
Ring B is selected from 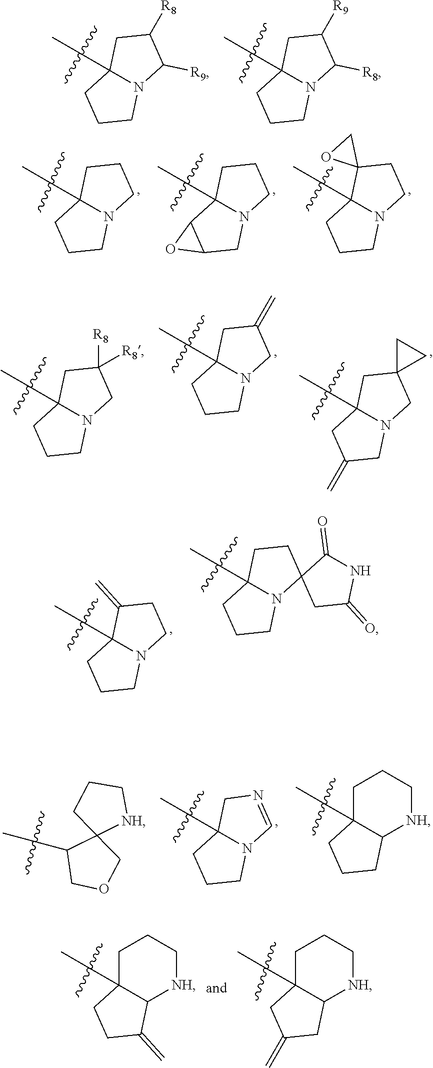
wherein Ring B is optionally substituted with 1, 2, 3, or 4 R₁₀;
L is selected from —C(R_{L1}R_{L2})—, wherein R_{L1} and R_{L2} are each independently selected from H, D, and C₁₋₃ alkyl;
R₁ and R₂ are each independently selected from oxo, H, F, Cl, Br, I, and CN;
each R₃ is independently selected from F, Cl, Br, I, OH, NH₂, CN, C₁₋₃ alkyl, C₁₋₃ alkoxy, C₁₋₃ alkylamino, di-C₁₋₃ alkylamino, C₂₋₄ alkenyl, C₂₋₄ alkynyl, and C₃₋₅ cycloalkyl, wherein the C₁₋₃ alkyl, C₁₋₃ alkoxy, C₁₋₃ alkylamino, di-C₁₋₃ alkylamino, C₂₋₄ alkenyl, C₂₋₄ alkynyl, and C₃₋₅ cycloalkyl are each independently optionally substituted with 1, 2, 3, 4, or 5 Rₐ;
each Rₐ is independently selected from D, F, Cl, Br, and I;
R₄, R₅, R₆, R₇, R_{6′}, and R_{7′} are each independently selected from oxo, H, F, Cl, Br, I, OH, NH₂, CN, C₁₋₃ alkyl, C₂₋₄ alkenyl, C₁₋₃ alkoxy, —C(═O)—Rₐ, —C(═O)—NR_{b1}R_{b2}, and ═NO(C₁₋₃ alkyl), wherein the C₁₋₃ alkyl, C₂₋₄ alkenyl, and C₁₋₃ alkoxy are each independently optionally substituted with 1, 2, 3, 4, or 5 R_{b};
or, R₆ and R₇ together with the carbon atoms to which they are attached form a 3-5 membered heterocyclyl,
each R_{b} is independently selected from D, F, Cl, Br, I, OH, NH₂, CN, C₁₋₃ alkoxy, and —C(═O)—NR_{b1}R_{b2};
R₈ is selected from H, F, Cl, Br, I, OH, NH₂, CN, C₁₋₃ alkyl, and C₁₋₃ alkoxy, wherein the C₁₋₃ alkyl and C₁₋₃ alkoxy are each independently optionally substituted with 1, 2, 3, 4, or 5 Rₐ;
or, R₈ and R_{8′} together with the carbon atom to which they are attached form a C₃₋₅ cycloalkyl or 3-5 membered heterocyclyl, wherein the C₃₋₅ cycloalkyl and 3-5 membered heterocyclyl are each independently optionally substituted with 1, 2, or 3 R₁₀;
R₉ is selected from —C(═O)—NR_{b3}R_{b4} and —CH₂R_{c};
each R₁₀ is independently selected from oxo, D, F, Cl, Br, I, OH, NH₂, CN, C₁₋₃ alkyl, C₁₋₃ alkoxy, C₁₋₃ alkylamino, di-C₁₋₃ alkylamino, —C(═O)—R_{d}, —S—R_{d}, —S(═O)—R_{d}, —S(═O)₂—R_{d}, —NH—C(═O)—R_{d}, C₆₋₁₀ aryl, and 5-10 membered heteroaryl, wherein the C₁₋₃ alkyl is optionally substituted with 1, 2, or 3 OH or F, and the C₆₋₁₀ aryl and 5-10 membered heteroaryl are each independently optionally substituted with 1, 2, 3, 4, or 5 Rₛ₁;
R_{b1} and R_{b2} are each independently selected from H, C₁₋₆ alkyl, C₁₋₆ alkoxy, C₃₋₁₀ cycloalkyl, 3-10 membered heterocyclyl, C₆₋₁₀ aryl, and 5-10 membered heteroaryl, wherein the C₁₋₆ alkyl, C₁₋₆ alkoxy, C₃₋₁₀ cycloalkyl, 3-10 membered heterocyclyl, C₆₋₁₀ aryl, and 5-10 membered heteroaryl are each independently optionally substituted with 1, 2, 3, or 4 Rₑ₂;
or, R_{b1} and R_{b2} together with the nitrogen atom to which they are attached form a 3-6 membered heterocyclyl, wherein the 3-6 membered heterocyclyl is optionally substituted with 1, 2, 3, or 4 Rₑ₁;
R_{b3} and R_{b4} are each independently selected from H, C₁₋₆ alkyl, C₁₋₆ alkoxy, C₃₋₁₀ cycloalkyl, 3-10 membered heterocyclyl, C₆₋₁₀ aryl, and 5-10 membered heteroaryl, wherein the C₁₋₆ alkyl, C₁₋₆ alkoxy, C₃₋₁₀ cycloalkyl, 3-10 membered heterocyclyl, C₆₋₁₀ aryl, and 5-10 membered heteroaryl are each independently optionally substituted with 1, 2, 3, or 4 Rₑ₂;
or, R_{b3} and R_{b4} together with the nitrogen atom to which they are attached form a 3-6 membered heterocyclyl, wherein the 3-6 membered heterocyclyl is optionally substituted with 1, 2, 3, or 4 Rₑ₂;
R_{c} is selected from F, Cl, Br, I, OH, NH₂, —(C═O)NR_{C1}R_{C2}, —O(C═O)NR_{C1}R_{C2}, —NR_{C0}(C═O)R_{C1}, and —NR_{C0}(C═O)NR_{C1}R_{C2};
R_{C0}, R_{C1}, and R_{C2} are each independently selected from H, C₁₋₆ alkyl, C₃₋₆ cycloalkyl, and 3-6 membered heterocyclyl;
R_{d} is selected from C₁₋₃ alkyl;
Rₑ₁ is selected from F, Cl, Br, I, OH, NH₂, NO₂, C₁₋₃ alkyl, C₁₋₃ alkylamino, di-C₁₋₃ alkylamino, CN, C₁₋₃ alkoxy, —S(═O)₂—(C₁₋₃ alkyl), —(C═O)(C₁₋₃ alkyl), —(C═O)O(C₁₋₃ alkyl), —(C═O)NH(C₁₋₃ alkyl), —(C═O)N(C₁₋₃ alkyl)₂, C₃₋₁₀ cycloalkyl, 3-10 membered heterocyclyl, C₆₋₁₀ aryl, and 5-10 membered heteroaryl;
Rₑ₂ is selected from F, Cl, Br, I, OH, NH₂, NO₂, C₁₋₃ alkyl, C₁₋₃ alkylamino, di-C₁₋₃ alkylamino, CN, C₁₋₃ alkoxy, —S(═O)₂—(C₁₋₃ alkyl), —(C═O)(C₁₋₃ alkyl), —(C═O)O(C₁₋₃ alkyl), —(C═O)NH(C₁₋₃ alkyl), —(C═O)N(C₁₋₃ alkyl)₂, C₃₋₁₀ cycloalkyl, 3-10 membered heterocyclyl, C₆₋₁₀ aryl, and 5-10 membered heteroaryl, wherein the C₃₋₁₀ cycloalkyl, 3-10 membered heterocyclyl, C₆₋₁₀ aryl, and 5-10 membered heteroaryl are each independently optionally substituted with 1, 2, 3, 4, or 5 Rₛ₁, and wherein the C₁₋₃ alkyl and C₁₋₃ alkoxy are each independently optionally substituted with 1, 2, 3, 4, or 5 Rₛ₂;
or, two Rₑ₂ together with the carbon atoms to which they are attached form a C₆ aryl or 5- or 6-membered heteroaryl;
Rₛ₁ is selected from oxo, F, Cl, Br, I, OH, NH₂, NO₂, C₁₋₆ alkyl, C₁₋₆ alkylamino, di-C₁₋₆ alkylamino, CN, C₁₋₆ alkoxy, —S(═O)₂—(C₁₋₃ alkyl), —(C═O)(C₁₋₃ alkyl), —(C=O)O(C₁₋₃ alkyl), —(C═O)NH(C₁₋₃ alkyl), and —(C═O)N(C₁₋₃ alkyl)₂;
Rₛ₂ is selected from F, Cl, Br, I, OH, NH₂, C₁₋₆ alkylamino, di-C₁₋₆ alkylamino, CN, C₁₋₆ alkoxy, —S(═O)₂—(C₁₋₃ alkyl), —(C═O)(C₁₋₃ alkyl), —(C=O)O(C₁₋₃ alkyl), —(C═O)NH(C₁₋₃ alkyl), and —(C═O)N(C₁₋₃ alkyl)₂; and
m is selected from 0, 1, 2, 3, 4, and 5;
provided that,
1) when Ring B is selected from 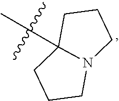 [SMILES: *C12CCCN1CCC2]
and the 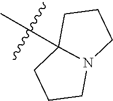 [SMILES: *C12CCCN1CCC2]
is substituted with 1 R₁₀, and R₁₀ is F, at least one of R₁, R₂, R₄, R₅, R₆, R₇, R_{6′}, and R_{7′} is not H;
2) when Ring B is selected from 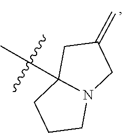 [SMILES: *C12CCCN1CC(=C)C2]
and the 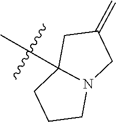 [SMILES: *C12CCCN1CC(=C)C2]
is optionally substituted with 1, 2, 3, or 4 R₁₀, at least one of R₁, R₂, R₄, R₅, R₆, R₇, R_{6′}, and R_{7′} is not H; and
3) the compound is not

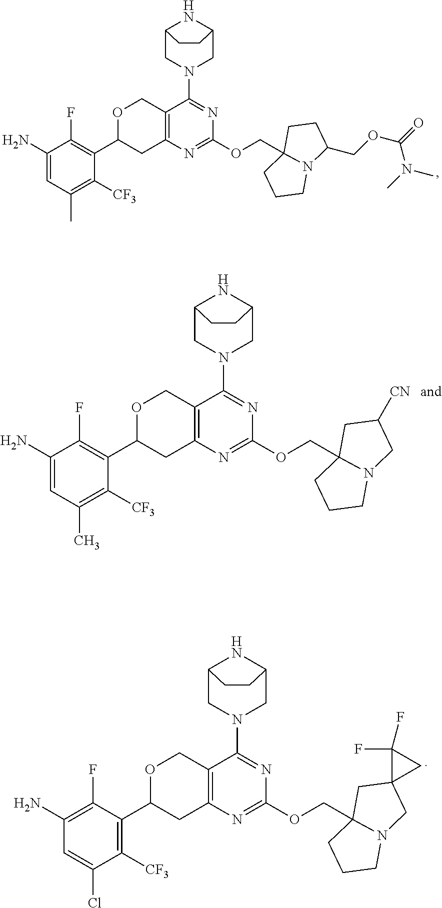 [SMILES: Cc1cc(N)c(F)c(C2Cc3nc(OCC45CCCN4C(COC(=O)N(C)C)CC5)nc(N4CC5CCC(C4)N5)c3CO2)c1C(F)(F)F ; Cc1cc(N)c(F)c(C2Cc3nc(OCC45CCCN4CC(C#N)C5)nc(N4CC5CCC(C4)N5)c3CO2)c1C(F)(F)F ; Nc1cc(Cl)c(C(F)(F)F)c(C2Cc3nc(OCC45CCCN4CC4(C5)CC4(F)F)nc(N4CC5CCC(C4)N5)c3CO2)c1F]

2. The compound or the stereoisomer or pharmaceutically acceptable salt thereof according claim 1, wherein R_{N} is H.

3. The compound or the stereoisomer or pharmaceutically acceptable salt thereof according claim 1, wherein R_{N} is C₁₋₃ alkyl optionally substituted with 1, 2, or 3 F or Cl.

4. A compound of Formula (l′) or a stereoisomer thereof, or a pharmaceutically acceptable salt thereof: 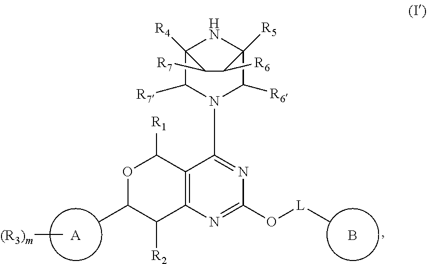
wherein,
Ring A is selected from C₆ aryl and 5-6 membered heteroaryl;
Ring B is selected from 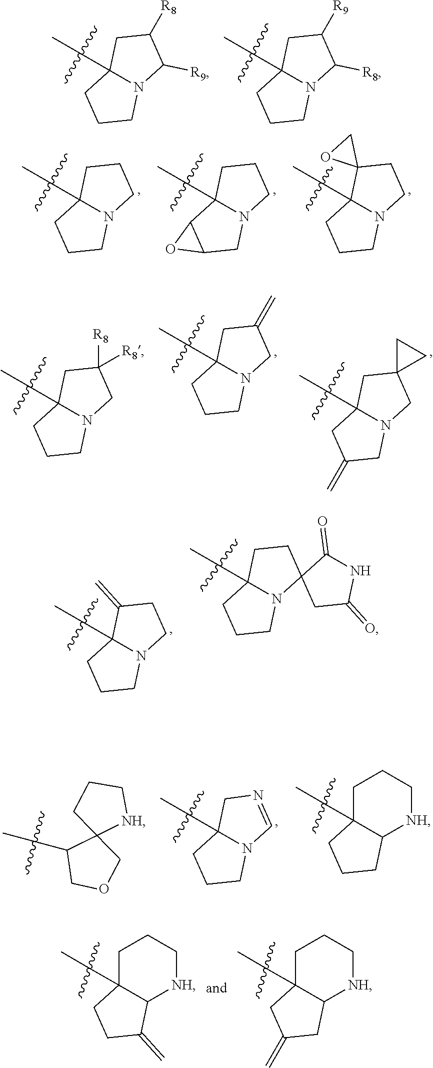
wherein Ring B is optionally substituted with 1, 2, 3, or 4 R₁₀;
L is selected from —CH₂—, wherein the —CH₂— is optionally substituted with 1 or 2 D;
R₁ and R₂ are each independently selected from oxo, H, F, Cl, Br, I, and CN;
each R₃ is independently selected from F, Cl, Br, I, OH, NH₂, CN, C₁₋₃ alkyl, C₁₋₃ alkoxy, C₁₋₃ alkylamino, di-C₁₋₃ alkylamino, C₂₋₄ alkenyl, C₂₋₄ alkynyl, and C₃₋₅ cycloalkyl, wherein the C₁₋₃ alkyl, C₁₋₃ alkoxy, C₁₋₃ alkylamino, di-C₁₋₃ alkylamino, C₂₋₄ alkenyl, C₂₋₄ alkynyl, and C₃₋₅ cycloalkyl are each independently optionally substituted with 1, 2, 3, 4, or 5 Rₐ;
each Rₐ is independently selected from D, F, Cl, Br, and I;
R₄, R₅, R₆, R₇, R_{6′}, and R_{7′} are each independently selected from oxo, H, F, Cl, Br, I, OH, NH₂, CN, C₁₋₃ alkyl, C₂₋₄ alkenyl, C₁₋₃ alkoxy, —C(═O)—R_{d}, —C(═O)—NR_{b1}R_{b2}, and ═NO(C₁₋₃ alkyl), wherein the C₁₋₃ alkyl, C₂₋₄ alkenyl, and C₁₋₃ alkoxy are each independently optionally substituted with 1, 2, 3, 4, or 5 R_{b};
or, R₆ and R₇ together with the carbon atoms to which they are attached form a 3-5 membered heterocyclyl;
each R_{b} is independently selected from D, F, Cl, Br, I, OH, NH₂, CN, C₁₋₃ alkoxy, and —C(═O)—NR_{b1}R_{b2};
R₈ is selected from H, F, Cl, Br, I, OH, NH₂, CN, C₁₋₃ alkyl, and C₁₋₃ alkoxy, wherein the C₁₋₃ alkyl and C₁₋₃ alkoxy are each independently optionally substituted with 1, 2, 3, 4, or 5 Rₐ;
or, R₈ and R_{8′} together with the carbon atom to which they are attached form a C₃₋₅ cycloalkyl or 3-5 membered heterocyclyl, wherein the C₃₋₅ cycloalkyl and 3-5 membered heterocyclyl are each independently optionally substituted with 1, 2, or 3 R₁₀;
R₉ is selected from —C(═O)—NR_{b1}R_{b2} and —CH₂R_{c};
each R₁₀ is independently selected from oxo, D, F, Cl, Br, I, OH, NH₂, CN, C₁₋₃ alkyl, C₁₋₃ alkoxy, C₁₋₃ alkylamino, di-C₁₋₃ alkylamino, —S—R_{d}, —S(═O)—R_{d}, —S(═O)₂—R_{d}, and —NH—C(═O)—R_{d}, wherein the C₁₋₃ alkyl is optionally substituted with 1, 2, or 3 OH;
R_{b1} and R_{b2} are each independently selected from H, C₁₋₆ alkyl, Cao cycloalkyl, 3-10 membered heterocyclyl, C₆₋₁₀ aryl, and 5-10 membered heteroaryl, wherein the C₁₋₆ alkyl, C₃₋₁₀ cycloalkyl, 3-10 membered heterocyclyl, C₆₋₁₀ aryl, and 5-10 membered heteroaryl are each independently optionally substituted with 1, 2, or 3 Rₑ₁;
or, R_{b1} and R_{b2} together with the nitrogen atom to which they are attached form a 3-6 membered heterocyclyl;
R_{c} is selected from F, Cl, Br, I, OH, NH₂, —O(C═O)NR_{C1}R_{C2}, —NR_{C0}(C═O)R_{C1}, and —NR_{C0}(C═O)NR_{C1}R_{C2};
R_{C0}, R_{C1}, and R_{C2} are each independently selected from H, C₁₋₆ alkyl, C₃₋₆ cycloalkyl, and 3-6 membered heterocyclyl;
R_{d} is selected from C₁₋₃ alkyl;
Rₑ₁ is selected from F, Cl, Br, I, OH, NH₂, C₁₋₃ alkylamino, di-C₁₋₃ alkylamino, CN, C₁₋₃ alkoxy, C₃₋₁₀ cycloalkyl, 3-10 membered heterocyclyl, C₆₋₁₀ aryl, and 5-10 membered heteroaryl; and
m is selected from 0, 1, 2, 3, 4, and 5;
provided that,
1) when Ring B is selected from 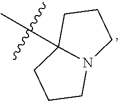 [SMILES: *C12CCCN1CCC2]
and the 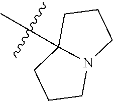 [SMILES: *C12CCCN1CCC2]
is substituted with 1 R₁₀, and R₁₀ is F, at least one of R₁, R₂, R₄, R₅, R₆, R₇, R_{6′}, and R_{7′} is not H;
2) when Ring B is selected from 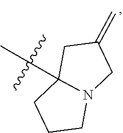 [SMILES: *C12CCCN1CC(=C)C2]
and the 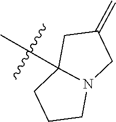 [SMILES: *C12CCCN1CC(=C)C2]
is optionally substituted with 1, 2, 3, or 4 R₁₀, at least one of R₁, R₂, R₄, R₅, R₆, R₇, R_{6′}, and R_{7′} is not H; and
3) the compound is not

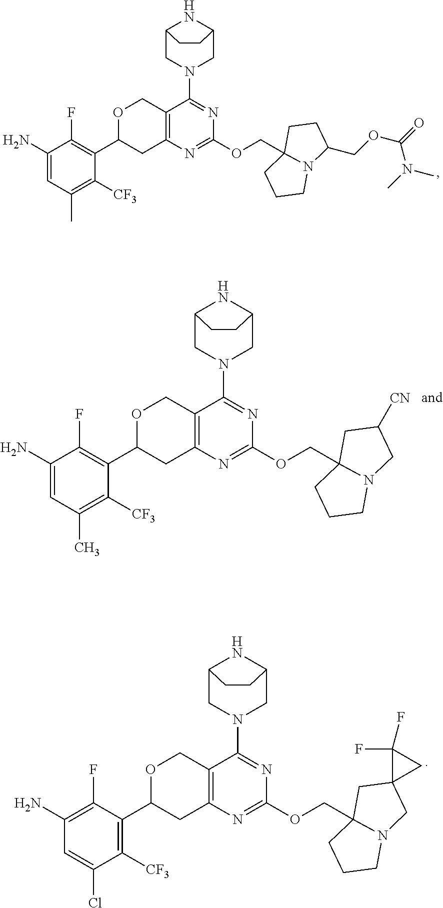 [SMILES: Cc1cc(N)c(F)c(C2Cc3nc(OCC45CCCN4C(COC(=O)N(C)C)CC5)nc(N4CC5CCC(C4)N5)c3CO2)c1C(F)(F)F ; Cc1cc(N)c(F)c(C2Cc3nc(OCC45CCCN4CC(C#N)C5)nc(N4CC5CCC(C4)N5)c3CO2)c1C(F)(F)F ; Nc1cc(Cl)c(C(F)(F)F)c(C2Cc3nc(OCC45CCCN4CC4(C5)CC4(F)F)nc(N4CC5CCC(C4)N5)c3CO2)c1F]

5. The compound or the stereoisomer or pharmaceutically acceptable salt thereof according to any one of claims 1-4, wherein Ring A is selected from C₆ aryl.

6. The compound or the stereoisomer or pharmaceutically acceptable salt thereof according to any one of claims 1-4, wherein Ring A is selected from 5-membered heteroaryl.

7. The compound or the stereoisomer or pharmaceutically acceptable salt thereof according to any one of claims 1-6, wherein Ring B is selected from 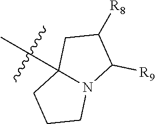
and 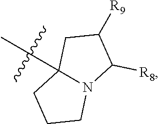
and is optionally substituted with 1, 2, 3, or 4 R₁₀.

8. The compound or the stereoisomer or pharmaceutically acceptable salt thereof according to any one of claims 1-7, wherein Ring B is selected from 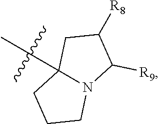
and is optionally substituted with 1, 2, 3, or 4 R₁₀.

9. The compound or the stereoisomer or pharmaceutically acceptable salt thereof according to claim 8, selected from a compound of Formula (I′-1-i) or a stereoisomers thereof, or a pharmaceutically acceptable salts thereof,

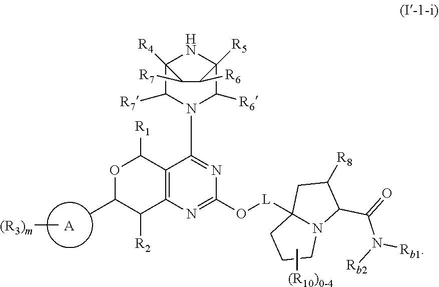

10. The compound or the stereoisomer or pharmaceutically acceptable salt thereof according to claim 8, selected from a compound of Formula (I′-2-i) or a stereoisomer thereof, or a pharmaceutically acceptable salt thereof,

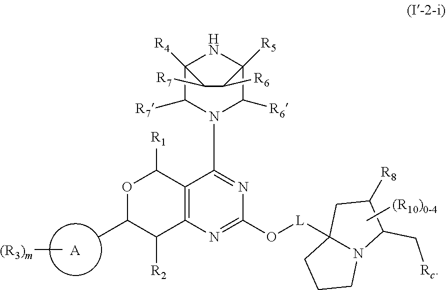

11. The compound or the stereoisomer or pharmaceutically acceptable salt thereof according to any one of claims 1-7, wherein Ring B is selected from 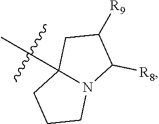
and is optionally substituted with 1, 2, 3, or 4 R₁₀.

12. The compound or the stereoisomer or pharmaceutically acceptable salt thereof according to claim 11, selected from a compound of Formula (I′-1-ii) or a stereoisomer thereof, or a pharmaceutically acceptable salt thereof,

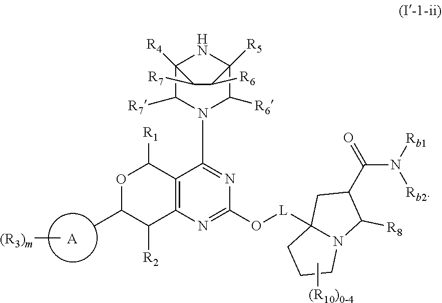

13. The compound or the stereoisomer or pharmaceutically acceptable salt thereof according to claim 11, selected from a compound of Formula (I′-2-ii) or a stereoisomer thereof, or a pharmaceutically acceptable salt thereof,

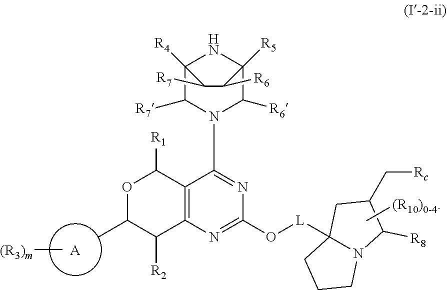

14. The compound or the stereoisomer or pharmaceutically acceptable salt thereof according to any one of claims 1-6, wherein Ring B is selected from 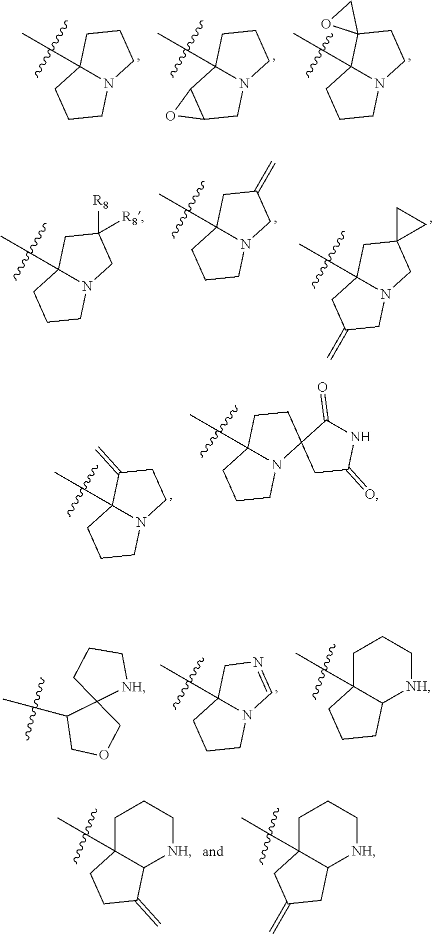
and is optionally substituted with 1, 2, 3, or 4 R₁₀.

15. The compound or the stereoisomer or pharmaceutically acceptable salt thereof according to claim 14, selected from a compound of Formula (I′-3), (I′-4), (I′-5), (l′-6), (I′-7), (I′-8), (l′-9), (I′-10), (I′-11), (l′-12), and (I′-13), or a stereoisomer thereof, or a pharmaceutically acceptable salt thereof,

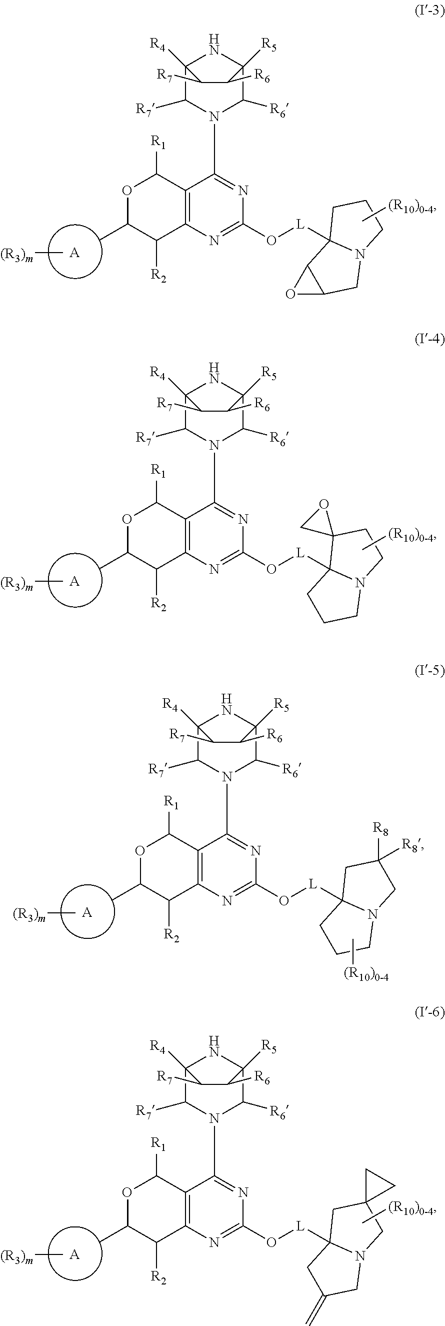

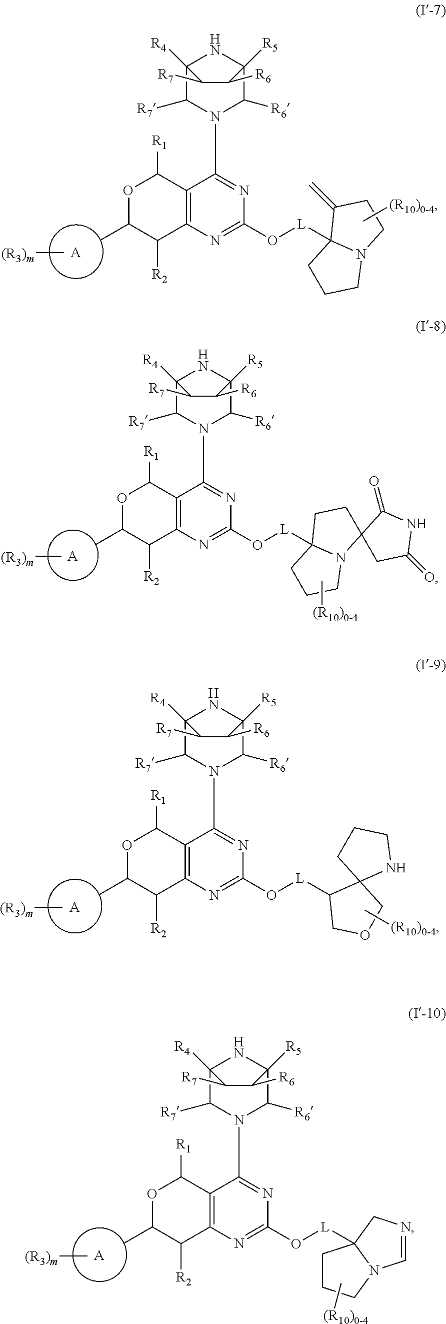

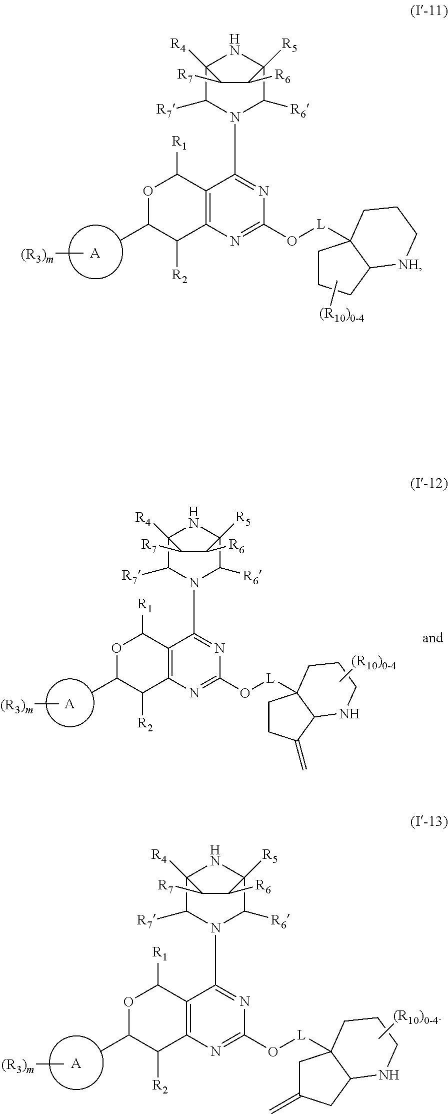

16. The compound or the stereoisomer or pharmaceutically acceptable salt thereof according to any one of claims 7-13 and 15, wherein R₄, R₅, R₆, R₇, R_{6′} and R_{7′} are selected from H.

17. The compound or the stereoisomer or pharmaceutically acceptable salt thereof according to claim 14, selected from a compound of Formula (I′-14) and (I′-15), or a stereoisomer thereof, or a pharmaceutically acceptable salt thereof,

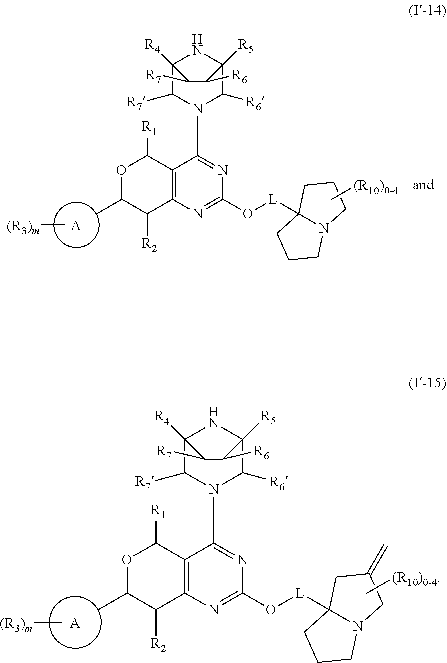

18. The compound or the stereoisomer or pharmaceutically acceptable salt thereof according to any one of claims 1-5 and 7-17, wherein Ring A is selected from phenyl, and is substituted with at least one R₃, and each R₃ is independently selected from F, OH, NH₂, CF₃, OCH₃, —C≡CCH₃, —C≡CCH₂F, —C≡CCHF₂, —C≡CCF₃, —C≡CCD₃, —C≡CH, —C≡CCl, —C≡CF and cyclopropyl.

19. The compound or the stereoisomer or pharmaceutically acceptable salt thereof according to any one of claims 1-5 and 7-17, wherein 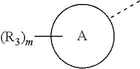
is selected from.

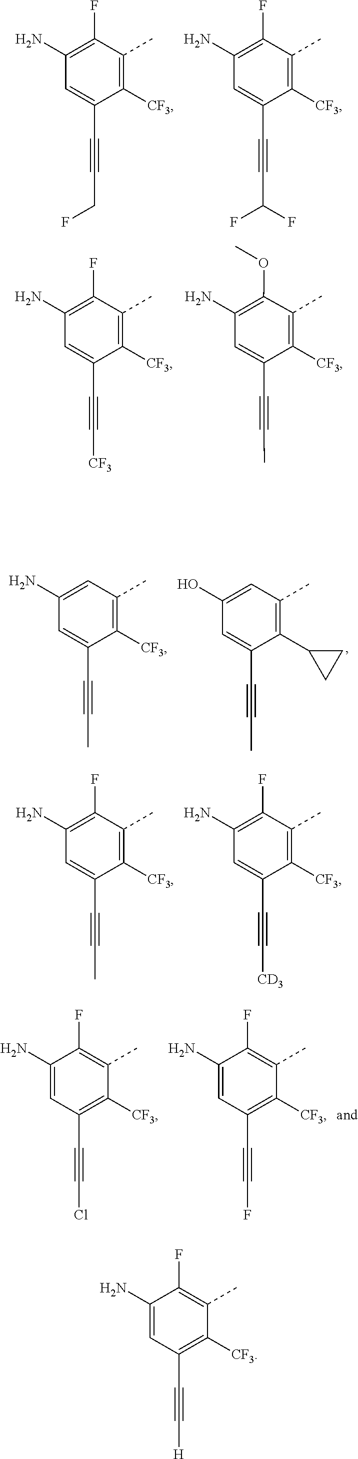

20. The compound or the stereoisomer or pharmaceutically acceptable salt thereof according to any one of claims 1-5 and 7-17, wherein 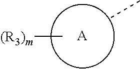
is selected from

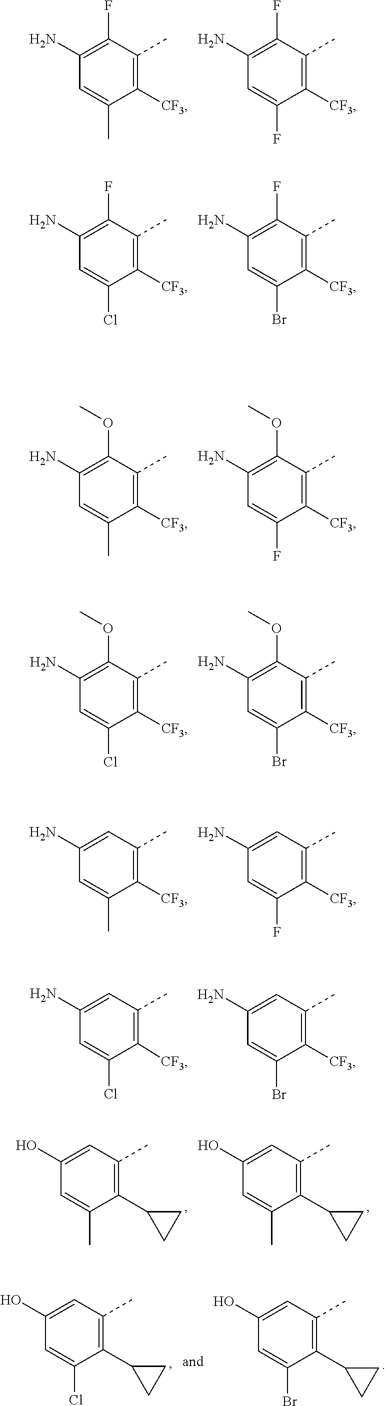

21. The compound or the stereoisomer or pharmaceutically acceptable salt thereof according to claim 19, selected from a compound of Formula (I′-1′-i) or a stereoisomer thereof, or a pharmaceutically acceptable salt thereof,

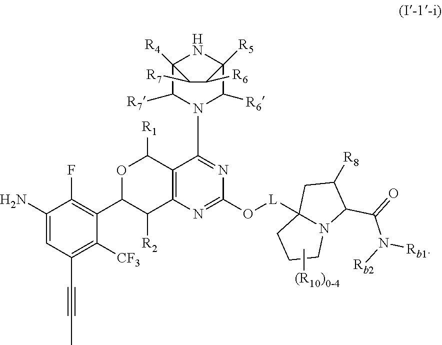

22. The compound or the stereoisomer or pharmaceutically acceptable salt thereof according to claim 19, selected from a compound of Formula (I′-1′-ii) or a stereoisomer thereof, or a pharmaceutically acceptable salt thereof,

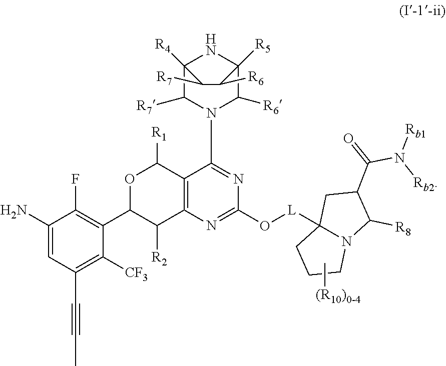

23. The compound or the stereoisomer or pharmaceutically acceptable salt thereof according to claim 19, selected from a compound of Formula (I′-2′-i) or a stereoisomer thereof, or a pharmaceutically acceptable salt thereof,

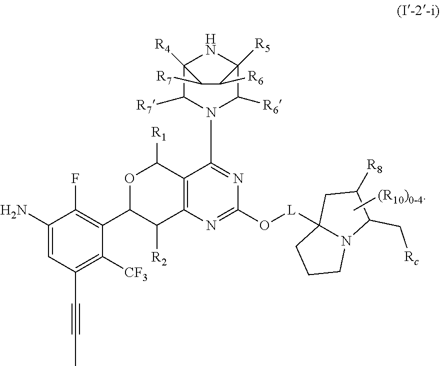

24. The compound or the stereoisomer or pharmaceutically acceptable salt thereof according to claim 19, selected from a compound of Formula (I′-2′-ii) or a stereoisomer thereof, or a pharmaceutically acceptable salt thereof,

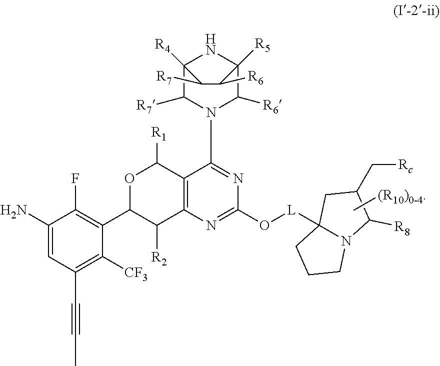

25. The compound or the stereoisomer or pharmaceutically acceptable salt thereof according to claim 19, selected from a compound of Formula (I′-3′), (I′-4′), (I′-5′), (I′-6′), (I′-7′), (I′-8′), (I′-9′), (I′-10′), (I′-11′), (I′-12′), and (I′-13′), or a stereoisomer thereof, or a pharmaceutically acceptable salt thereof,

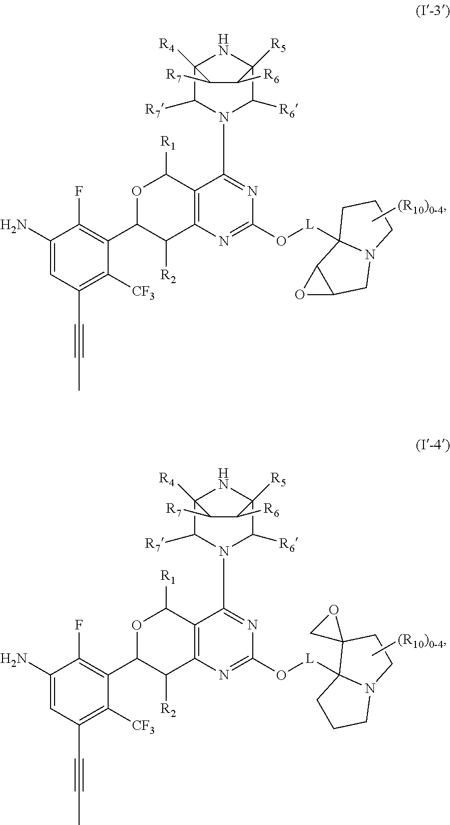

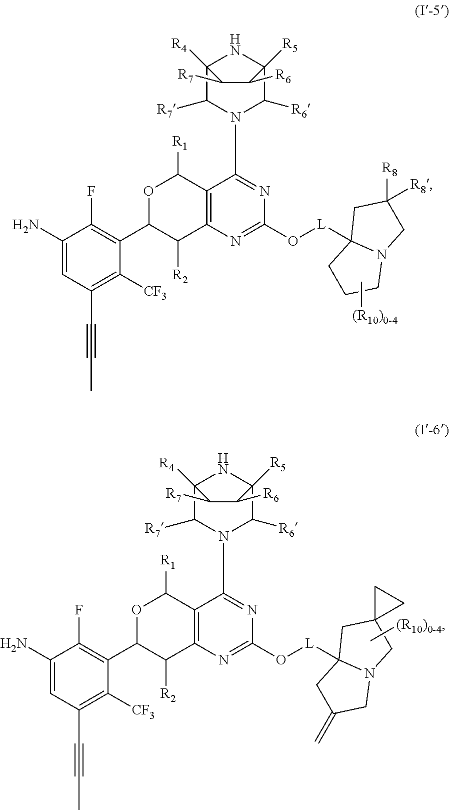

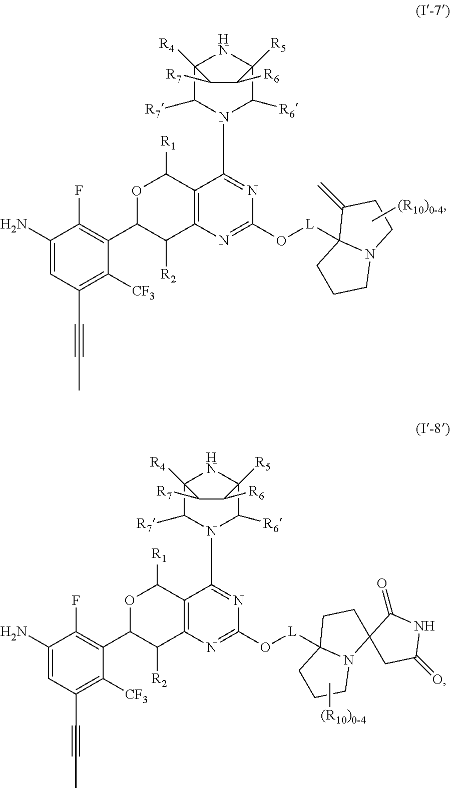

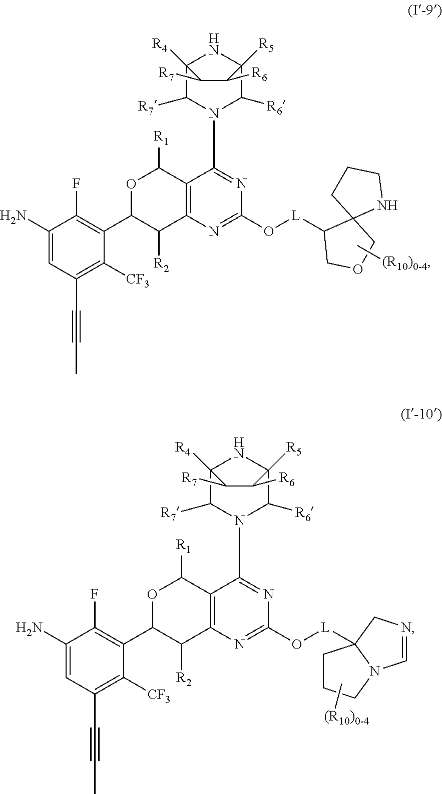

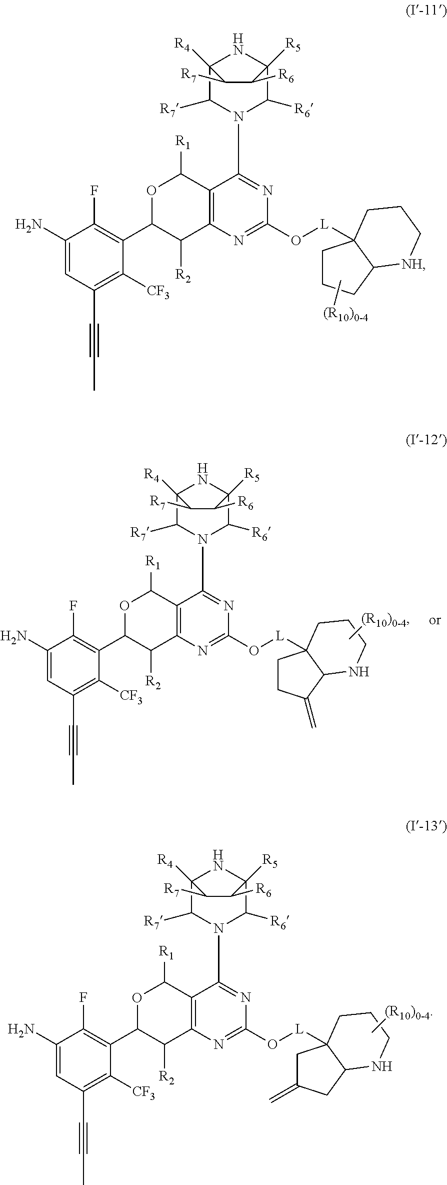

26. The compound or the stereoisomer or pharmaceutically acceptable salt thereof according to claim 25, wherein R₄, R₅, R₆, R₇, R_{6′}, and R_{7′} are selected from H.

27. The compound or the stereoisomer or pharmaceutically acceptable salt thereof according to claim 19, selected from a compound of Formula (I′-14′) and (I′-15′), or a stereoisomer thereof, or a pharmaceutically acceptable salt thereof,

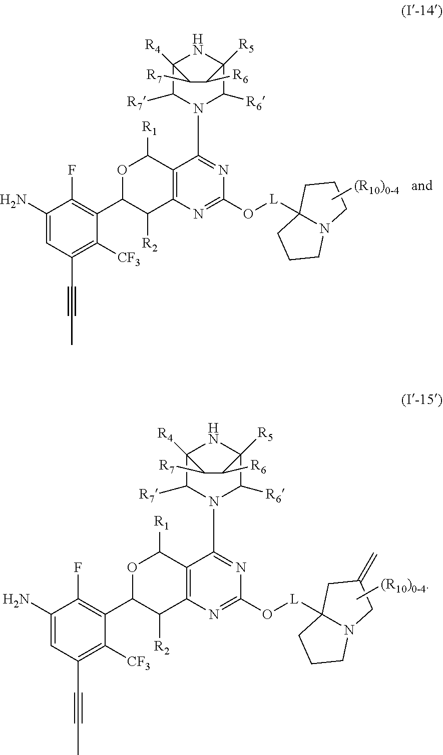

28. The compound or the stereoisomer or pharmaceutically acceptable salt thereof according to any one of claims 1-27, wherein L is selected from —C(R_{L1}R_{L2})—, and R_{L1} and R_{L2} are each independently selected from H and D.

29. The compound or the stereoisomer or pharmaceutically acceptable salt thereof according to any one of claims 1-27, wherein L is selected from —C(R_{L1}R_{L2})—, and R_{L1} and R_{L2} are each selected from H.

30. The compound or the stereoisomer or pharmaceutically acceptable salt thereof according to any one of claims 1-27, wherein L is selected from —C(R_{L1}R_{L2})—, and at least one of R_{L1} and R_{L2} is selected from C₁₋₃ alkyl.

31. The compound or the stereoisomer or pharmaceutically acceptable salt thereof according to any one of claims 1-30, wherein R₁ and R₂ are selected from H.

32. The compound or the stereoisomer or pharmaceutically acceptable salt thereof according to claim 1, selected from Table 5 or Table 5a.

33. The compound or the stereoisomer or pharmaceutically acceptable salt thereof according to claim 32, selected from Table 6 and Table 6a.

34. Use of the compound or the stereoisomer or pharmaceutically acceptable salt thereof according to any one of claims 1-33, in the preparation of a medicament for the treatment of a disease or disorder associated with a KRAS mutation.

35. The compound or the stereoisomer or pharmaceutically acceptable salt thereof according to any one of claims 1-33, for use in treating a disease or disorder associated with a KRAS mutation.

36. A method of treating a disease or disorder associated with a KRAS mutation, comprising 2dministering to a subject in need thereof the compound or the stereoisomer or pharmaceutically acceptable salt thereof according to any one of claims 1-33.

37. The use of claim 34, the compound for use of claim 35, or the method of claim 36, wherein the KRAS mutation is a KRAS G12D mutation.
